SEQUENCE LISTING (80 sequences)

SEQ ID NO: 1 - DNA, 5578 bp, Homo sapiens
  source 1..5578: mol_type = unassigned DNA
gttaacaggcgcgtcccggccaggcggagacgcggccgcggccatgggcgggcgcgggcgcgcggggcggcggtgagggcggctggcggggccgggggcgccgggggggcgcgcgggccgagccgggcctgagccgggcccgcggaccgagctgggagaggggttccggcccccgacgtgctggcgcgggaaaatgttggagatctgcctgaagctggtgggctgcaaatccaagaaggggctgtcctcgtcctccagctgttatctggaagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccagcggccactcaggacttcagcaagctcctcagttcggtgaaggaaatcagtgacatagtgcagaggtagcagcagtcaggggtcaggtgtcaggcccgtcggagctgcctgcagcacatgcgggctcgcccatacccgtgacagtggctgacaagggactagtgagtcagcaccttggcccaggagctctgcgccaggcagagctgagggccctgtggagtccagctctactacctacgtttgcaccgcctgccctcccgcaccttcctcctccccgctccgtctctgtcctcgaattttatctgtggagttcctgctccgtggactgcagtcggcatgccaggacccgccagccccgctcccacctagtgccccagactgagctctccaggccaggtgggaacggctgatgtggactgtctttttcatttttttctctctggagcccctcctcccccggctgggcctccttcttccacttctccaagaatggaagcctgaactgaggccttgtgtgtcaggccctctgcctgcactccctggccttgcccgtcgtgtgctgaagacatgtttcaagaaccgcatttcgggaagggcatgcacgggcatgcacacggctggtcactctgccctctgctgctgcccggggtggggtgcactcgccatttcctcacgtgcaggacagctcttgatttgggtggaaaacagggtgctaaagccaaccagcctttgggtcctgggcaggtgggagctgaaaaggatcgaggcatggggcatgtcctttccatctgtccacatccccagagcccagctcttgctctcttgtgacgtgcactgtgaatcctggcaagaaagcttgagtctcaagggtggcaggtcactgtcactgccgacatccctcccccagcagaatggaggcaggggacaagggaggcagtggctagtggggtgaacagctggtgccaaatagccccagactgggcccaggcaggtctgcaagggcccagagtgaaccgtcctttcacacatctgggtgccctgaaagggcccttcccctcccccactcctctaagacaaagtagattcttacaaggccctttcctttggaacaagacagccttcacttttctgagttcttgaagcatttcaaagccctgcctctgtgtagccgccctgagagagaatagagctgccactgggcacctgcgcacaggtgggaggaaagggcctggccagtcctggtcctggctgcactcttgaactgggcgaatgtcttatttaattaccgtgagtgacatagcctcatgttctgtgggggtcatcagggagggttaggaaaaccacaaacggagcccctgaaagcctcacgtatttcacagagcacgcctgccatcttctccccgaggctgccccaggccggagcccagatacgggggctgtgactctgggcagggacccggggtctcctggaccttgacagagcagctaactccgagagcagtgggcaggtggccgcccctgaggcttcacgccgggagaagccaccttcccaccccttcataccgcctcgtgccagcagcctcgcacaggccctagctttacgctcatcacctaaacttgtactttatttttctgatagaaatggtttcctctggatcgttttatgcggttcttacagcacatcacctctttgcccccgacggctgtgacgcagccggagggaggcactagtcaccgacagcggccttgaagacagagcaaagcgcccacccaggtcccccgactgcctgtctccatgaggtactggtcccttccttttgttaacgtgatgtgccactatattttacacgtatctcttggtatgcatcttttatagacgctcttttctaagtggcgtgtgcatagcgtcctgccctgccccctcgggggcctgtggtggctccccctctgcttctcggggtccagtgcattttgtttctgtatatgattctctgtggttttttttgaatccaaatctgtcctctgtagtattttttaaataaatcagtgtttacattagaa

SEQ ID NO: 2 - DNA, 4137 bp, Homo sapiens
  source 1..4137: mol_type = unassigned DNA
atggccgccgccgatgccgaggcagttccggcgaggggggagcctcagcaggattgctgtgtgaaaaccgagctgctgggagaagagacacctatggctgccgatgaaggctcagcagagaaacaggcaggagaggcccacatggctgcggacggtgagaccaatgggtcttgtgaaaacagcgatgccagcagtcatgcaaatgctgcaaagcacactcaggacagcgcaagggtcaacccccaggatggcaccaacacactaactcggatagcggaaaatggggtttcagaaagagactcagaagcggcgaagcaaaaccacgtcactgccgacgactttgtgcagacttctgtcatcggcagcaacggatacatcttaaataagccggccctacaggcacagcccttgaggactaccagcactctggcctcttcgctgcctggccatgctgcaaaaacccttcctggaggggctggcaaaggcaggactccaagcgcttttccccagacgccagccgccccaccagccacccttggggaggggagtgctgacacagaggacaggaagctcccggcccctggcgccgacgtcaaggtccacagggcacgcaagaccatgccgaagtccgtcgtgggcctgcatgcagccagtaaagatcccagagaagttcgagaagctagagatcataaggaaccaaaagaggagatcaacaaaaacatttctgactttggacgacagcagcttttaccccccttcccatcccttcatcagtcgctacctcagaaccagtgctacatggccaccacaaaatcacagacagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccagcggccactcaggacttcagcaagctcctcagttcggtgaaggaaatcagtgacatagtgcagaggtag

SEQ ID NO: 3 - DNA, 3144 bp, Homo sapiens
  source 1..3144: mol_type = unassigned DNA
atggcggacgtgataaatgtcagtgtgaacctggaggccttttcccaggccattagtgccatccaggcgctgcgctccagcgtgagcagggtgttcgactgcctgaaggatgggatgcggaacaaggagacgctggagggccgggagaaggcctttattgcgcacttccaggacaacttacattcggtcaaccgggacctcaatgagctggaacgtctgagcaatctggtaggcaagccatctgagaaccatcctcttcataacagtgggctgttaagcctggatcctgtgcaggacaaaactcctctctatagtcaactccttcaagcatataagtggtcaaacaagttgcagtaccatgcaggactagcatctggccttttaaatcagcagtcattgaagcgttccgctaatcagatgggagtatctgccaaacgtagaccaaaggctcagcccacaactcttgtcctaccacctcaatatgttgatgatgtgatcagccgcattgacaggatgtttcctgaaatgtccatccacttatccagacccaatggaacatcagcaatgcttctggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccagcggccactcaggacttcagcaagctcctcagttcggtgaaggaaatcagtgacatagtgcagaggtag

SEQ ID NO: 4 - DNA, 4200 bp, Homo sapiens
  source 1..4200: mol_type = unassigned DNA
atgtgggcaacctgctgcaactggttctgcctggatggacagcctgaggaggtcccaccaccccagggagccaggatgcaggcctattccaaccctgggtacagctccttcccttccccaacaggcttggaaccaagctgcaagtcctgtggggctcactttgcaaacacggccaggaagcagacctgcttggactgtaagaaaaatttttgcatgacctgttcgagccaagtagggaatgggccccgcctctgccttctctgccaacggtttcgagctacagcctttcagcgagaggagctcatgaagatgaaggtgaaggacttgagggactatctcagcctccatgacatctctaccgaaatgtgccgggagaaagaagagctggtgctcttggtccttggccagcagcctgtaatctcccaggaggacaggactcgtgcctccaccttgtccccagactttcctgagcagcaggccttcctgacccagcctcactccagcatggttccacctacctcacccaacctcccctcttcatctgcacaagccacctctgttcccccagcccaggttcaggagaatcagcaggccaatggccatgtgtctcaggatcaagaggaacccgtctacctggagagcgtggccagagtacctgctgaggatgagacccagtctattgactcagaggacagctttgtcccaggccgaagggcctctctgtctgacctgactgacctggaggacattgaaggcctgacagtgcggcagctgaaagagatcttggctcgcaactttgtcaactacaagggctgctgtgagaagtgggagctgatggagagagtgacccggctatacaaggatcagaaaggactccagcacctggaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccagcggccactcaggacttcagcaagctcctcagttcggtgaaggaaatcagtgacatagtgcagaggtag

SEQ ID NO: 5 - DNA, 4260 bp, Homo sapiens
  source 1..4260: mol_type = unassigned DNA
atgctgcttggagcctctctggtgggggtgctgctgttctccaagctggtgctgaaactgccctggacccaggtgggattctccctgttgttcctctacttgggatctggcggctggcgcttcatccgggtcttcatcaagaccatcaggcgcgatatctttggcggcctggtcctcctgaaggtgaaggcaaaggtgcgacagtgcctgcaggagcggcggacagtgcccattttgtttgcctctaccgttcggcgccaccccgacaagacggccctgatcttcgagggcacagatacccactggaccttccgccagctggatgagtactcaagcagtgtagccaacttcctgcaggcccggggcctggcctcgggcgatgtggctgccatcttcatggagaaccgcaatgagttcgtgggcctatggctgggcatggccaagctcggtgtggaggcagccctcatcaacaccaacctgcggcgggatgctctgctccactgcctcaccacctcgcgcgcacgggcccttgtctttggcagcgaaatggcctcagccatctgtgaggtccatgccagcctggacccctcgctcagcctcttctgctctggctcctgggagcccggtgcggtgcctccaagcacagaacacctggaccctctgctgaaagatgctcccaagcaccttcccagttgccctgacaagggcttcacagataaactgttctacatctacacatccggcaccacagggctgcccaaggccgccatcgtggtgcacagcaggtattaccgcatggctgccctggtgtactatggattccgcatgcggcccaacgacatcgtctatgactgcctccccctctaccactcagcaggaaacatcgtgggaatcggccagtgcctgctgcatggcatgacggtggtgattcggaagaagttctcagcctcccggttctgggacgattgtatcaagtacaactgcacgattgtgcagtacattggtgaactgtgccgctacctcctgaaccagccaccgcgggaggcagaaaaccagcaccaggttcgcatggcactaggcaatggcctccggcagtccatctggaccaacttttccagccgcttccacataccccaggtggctgagttctacggggccacagagtgcaactgtagcctgggcaacttcgacagccaggtgggggcctgtggtttcaatagccgcatcctgtccttcgtgtaccccatccggttggtacgtgtcaacgaggacaccatggagctgatccgggggcccgacggcgtctgcattccctgccagccaggtgagccgggccagctggtgggccgcatcatccagaaagaccccctgcgccgcttcgatggctacctcaaccagggcgccaacaacaagaagattgccaaggatgtcttcaagaagggggaccaggcctaccttactggtgatgtgctggtgatggacgagctgggctacctgtacttccgagaccgcactggggacacgttccgctggaaaggtgagaacgtgtccaccaccgaggtggaaggcacactcagccgcctgctggacatggctgacgtggccgtgtatggtgtcgaggtgccaggaaccgagggccgggccggaatggctgctgtggccagccccactggcaactgtgacctggagcgctttgctcaggtcttggagaaggaactgcccctgtatgcgcgccccatcttcctgcgcctcctgcctgagctgcacaaaacaggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccagcggccactcaggacttcagcaagctcctcagttcggtgaaggaaatcagtgacatagtgcagaggtag

SEQ ID NO: 6 - DNA, 6021 bp, Homo sapiens
  source 1..6021: mol_type = unassigned DNA
atggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctggagtccactaaagcgagtgagctggacttggaaaagggcttggagatgagaaaatgggtcctgtcgggaatcctggctagcgaggagacttacctgagccacctggaggcactgctgctgcccatgaagcctttgaaagccgctgccaccacctctcagccggtgctgacgagtcagcagatcgagaccatcttcttcaaagtgcctgagctctacgagatccacaaggagttctatgatgggctcttcccccgcgtgcagcagtggagccaccagcagcgggtgggcgacctcttccagaagctggccagccagctgggtgtgtaccgggccttcgtggacaactacggagttgccatggaaatggctgagaagtgctgtcaggccaatgctcagtttgcagaaatctccgagaacctgagagccagaagcaacaaagatgccaaggatccaacgaccaagaactctctggaaactctgctctacaagcctgtggaccgtgtgacgaggagcacgctggtcctccatgacttgctgaagcacactcctgccagccaccctgaccaccccttgctgcaggacgccctccgcatctcacagaacttcctgtccagcatcaatgaggagatcacaccccgacggcagtccatgacggtgaagaagggagagcaccggcagctgctgaaggacagcttcatggtggagctggtggagggggcccgcaagctgcgccacgtcttcctgttcaccgacctgcttctctgcaccaagctcaagaagcagagcggaggcaaaacgcagcagtatgactgcaaatggtacattccgctcacggatctcagcttccagatggtggatgaactggaggcagtgcccaacatccccctggtgcccgatgaggagctggacgctttgaagatcaagatctcccagatcaagaatgacatccagagagagaagagggcgaacaagggcagcaaggctacggagaggctgaagaagaagctgtcggagcaggagtcactgctgctgcttatgtctcccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaataaggaagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccagcggccactcaggacttcagcaagctcctcagttcggtgaaggaaatcagtgacatagtgcagaggtag

SEQ ID NO: 7 - DNA, 6096 bp, Homo sapiens
  source 1..6096: mol_type = unassigned DNA
atggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctggagtccactaaagcgagtgagctggacttggaaaagggcttggagatgagaaaatgggtcctgtcgggaatcctggctagcgaggagacttacctgagccacctggaggcactgctgctgcccatgaagcctttgaaagccgctgccaccacctctcagccggtgctgacgagtcagcagatcgagaccatcttcttcaaagtgcctgagctctacgagatccacaaggagttctatgatgggctcttcccccgcgtgcagcagtggagccaccagcagcgggtgggcgacctcttccagaagctggccagccagctgggtgtgtaccgggccttcgtggacaactacggagttgccatggaaatggctgagaagtgctgtcaggccaatgctcagtttgcagaaatctccgagaacctgagagccagaagcaacaaagatgccaaggatccaacgaccaagaactctctggaaactctgctctacaagcctgtggaccgtgtgacgaggagcacgctggtcctccatgacttgctgaagcacactcctgccagccaccctgaccaccccttgctgcaggacgccctccgcatctcacagaacttcctgtccagcatcaatgaggagatcacaccccgacggcagtccatgacggtgaagaagggagagcaccggcagctgctgaaggacagcttcatggtggagctggtggagggggcccgcaagctgcgccacgtcttcctgttcaccgacctgcttctctgcaccaagctcaagaagcagagcggaggcaaaacgcagcagtatgactgcaaatggtacattccgctcacggatctcagcttccagatggtggatgaactggaggcagtgcccaacatccccctggtgcccgatgaggagctggacgctttgaagatcaagatctcccagatcaagaatgacatccagagagagaagagggcgaacaagggcagcaaggctacggagaggctgaagaagaagctgtcggagcaggagtcactgctgctgcttatgtctcccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaataaggaagatgatgagtctccggggctctatgggtttctgaatgtcatcgtccactcagccactggatttaagcagagttcaaaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccagcggccactcaggacttcagcaagctcctcagttcggtgaaggaaatcagtgacatagtgcagaggtag

SEQ ID NO: 8 - DNA, 6096 bp, Homo sapiens; identical to SEQ ID NO: 7
  source 1..6096: mol_type = unassigned DNA

SEQ ID NO: 9 - DNA, 6096 bp, Homo sapiens; identical to SEQ ID NO: 7
  source 1..6096: mol_type = unassigned DNA

SEQ ID NO: 10 - DNA, 6096 bp, Homo sapiens; identical to SEQ ID NO: 7
  source 1..6096: mol_type = unassigned DNA

SEQ ID NO: 11 - DNA, 6096 bp, Homo sapiens; identical to SEQ ID NO: 7
  source 1..6096: mol_type = unassigned DNA

SEQ ID NO: 12 - DNA, 6096 bp, Homo sapiens; identical to SEQ ID NO: 7
  source 1..6096: mol_type = unassigned DNA

SEQ ID NO: 13 - DNA, 6021 bp, Homo sapiens; identical to SEQ ID NO: 6
  source 1..6021: mol_type = unassigned DNA

SEQ ID NO: 14 - DNA, 6096 bp, Homo sapiens; identical to SEQ ID NO: 7
  source 1..6096: mol_type = unassigned DNA

SEQ ID NO: 15 - DNA, 6096 bp, Homo sapiens; identical to SEQ ID NO: 7
  source 1..6096: mol_type = unassigned DNA

SEQ ID NO: 16 - DNA, 6096 bp, Homo sapiens; identical to SEQ ID NO: 7
  source 1..6096: mol_type = unassigned DNA

SEQ ID NO: 17 - DNA, 6096 bp, Homo sapiens; identical to SEQ ID NO: 7
  source 1..6096: mol_type = unassigned DNA

SEQ ID NO: 18 - DNA, 6096 bp, Homo sapiens; identical to SEQ ID NO: 7
  source 1..6096: mol_type = unassigned DNA

SEQ ID NO: 19 - DNA, 9354 bp, Homo sapiens
  source 1..9354: mol_type = unassigned DNA
atgggagacgagatggatgccatgattcccgagcgggagatgaaggattttcagtttagagcgctaaagaaggtgagaatctttgactcccctgaggaattgcccaaggaacgctcgagtctgcttgctgtgtccaacaaatatggtctggtcttcgctggtggagccagtggcttgcagatttttcctactaaaaatcttcttattcaaaataaacccggagatgatcccaacaaaatagttgataaagtccaaggcttgctagttcctatgaaattcccaatccatcacctggccttgagctgtgataacctcacactctctgcgtgcatgatgtccagtgaatatggttccattattgctttttttgatgttcgcacattctcaaatgaggctaaacagcaaaaacgcccatttgcctatcataagcttttgaaagatgcaggaggcatggtgattgatatgaagtggaaccccactgtcccctccatggtggcagtttgtctggctgatggtagtattgctgtcctgcaagtcacggaaacagtgaaagtatgtgcaactcttccttccacggtagcagtaacctctgtgtgctggagccccaaaggaaagcagctggcagtgggaaaacagaatggaactgtggtccagtatcttcctactttgcaggaaaaaaaagtcattccttgtcctccgttttatgagtcagatcatcctgtcagagttctggatgtgctgtggattggtacctacgtcttcgccatagtgtatgctgctgcagatgggaccctggaaacgtctccagatgtggtgatggctctactaccgaaaaaagaagaaaagcacccagagatatttgtgaactttatggagccctgttatggcagctgcacggagagacagcatcattactacctcagttacattgaggaatgggatttagtgctggcagcatctgcggcttcaacagaagttagtatccttgctcgacaaagtgatcagattaattgggaatcttggctactggaggattctagtcgagctgaattgcctgtgacagacaagagtgatgactccttgcccatgggagttgtcgtagactatacaaaccaagtggaaatcaccatcagtgatgaaaagactcttcctcctgctccagttctcatgttactttcaacagatggtgtgctttgtccattttatatgattaatcaaaatcctggggttaagtctctcatcaaaacaccagagcgactttcattagaaggagagcgacagcccaagtcaccaggaagtactcccactaccccaacctcctctcaagccccacagaaactggatgcttctgcagctgcagcccctgcctctctgccaccttcatcacctgctgctcccattgccactttttctttgcttcctgctggtggagcccccactgtgttctcctttggttcttcatctttgaagtcatctgctacggtcactggggagcccccttcatattccagtggctccgacagctccaaagcagccccaggccctggcccatcaaccttctcttttgttcccccttctaaagcctccctagcccccacccctgcagcgtctcctgtggctccatcagctgcttcattctcctttggatcatctggttttaagcctaccctggaaagcacaccagtgccaagtgtgtctgctccaaatatagcaatgaagccctccttcccaccctcaacctctgctgtcaaagtcaaccttagtgaaaagtttactgctgcagctacctctactcctgttagtagctcccagagcgcacccccgatgtcgccattctcttctgcctccaagccagctgcttctggaccactcagccaccccacacctctctcagcaccacctagttccgtgccattgaagtcctcagtcttgccctcaccatcaggacgatctgctcagggcagttcaagcccagtgccctcaatggtacagaaatcacccaggataacccctccagcggcaaagccaggctctccccaggcaaagtcacttcagcctgctgttgcagaaaagcagggacatcagtggaaagattcagatcctgtaatggctggaattggggaggagattgcacactttcagaaggagttggaagagttaaaagcccgaacttccaaagcctgtttccaagtgggcacttctgaggagatgaagatgctgcgaacagaatcagatgacttgcatacctttcttttggagattaaagagaccacagagtcgcttcatggagatataagtagcctgaaaacaactttacttgagggctttgctggtgttgaggaagccagagaacaaaatgaaagaaatcgtgactctggttatctgcatttgctttataaaagaccactggatcccaagagtgaagctcagcttcaggaaattcggcgccttcatcagtatgtgaaatttgctgtccaagatgtgaatgatgttctagacttggagtgggatcagcatctggaacaaaagaaaaaacaaaggcacctgcttgtgccagagcgagagacactgtttaacaccctagccaacaatcgggaaatcatcaaccaacagaggaagaggctgaatcacctggtggatagtcttcagcagctccgcctttacaaacagacttccctgtggagcctgtcctcggctgttccttcccagagcagcattcacagttttgacagtgacctggaaagcctgtgcaatgctttgttgaaaaccaccatagaatctcacaccaaatccttgcccaaagtaccagccaaactgtcccccatgaaacaggcacaactgagaaacttcttggccaagaggaagaccccaccagtgagatccactgctccagccagcctgtctcgatcagcctttctgtctcagagatattatgaagacttggatgaagtcagctcaacgtcatctgtctcccagtctctggagagtgaagatgcacggacgtcctgtaaagatgacgaggcagtggttcaggcccctcggcacgcccccgtggttcgcactccttccatccagcccagtctcttgccccatgcagcaccttttgctaaatctcacctggttcatggttcttcacctggtgtgatgggaacttcagtggctacatctgctagcaaaattattcctcaaggggccgatagcacaatgcttgccacgaaaaccgtgaaacatggtgcacctagtccttcccaccccatctcagccccgcaggcagctgccgcagcagcactcaggcggcagatggccagtcaggcaccagctgtaaacactttgactgaatcaacgttgaagaatgtccctcaagtggtaaatgtgcaggaattgaagaataaccctgcaaccccttctacagccatgggttcttcagtgccctactccacagccaaaacacctcacccagtgttgaccccagtggctgctaaccaagccaagcaggggtctctaataaattcccttaagccatctgggcctacaccagcatccggtcagttatcatctggtgacaaagcttcagggacagccaagatagaaacagctgtgacttcaaccccatctgcttctgggcagttcagcaagcctttctcattttctccatcagggactggctttaattttgggataatcacaccaacaccgtcttctaatttcactgctgcacaaggggcaacaccctccactaaagagtcaagccagccggacgcattctcatctggtgggggaagcaaaccttcttatgaggccattcctgaaagctcacctccctcaggaatcacatccgcatcaaacaccaccccaggagaacctgccgcatctagcagcagacctgtggcaccttctggaactgctctttccaccacctctagtaagctggaaaccccaccgtccaagctgggagagcttctgtttccaagttctttggctggagagactctgggaagtttttcaggactgcgggttggccaagcagatgattctacaaaaccaaccaataaggcttcatccacaagcctaactagtacccagccaaccaagacgtcaggcgtgccctcagggtttaattttactgcccccccggtgttagggaagcacacggagccccctgtgacatcctctgcaaccaccacctcagtagcaccaccagcagccaccagcacttcctcaactgccgtttttggcagtctgccagtcaccagtgcaggatcctctggggtcatcagttttggtgggacatctctaagtgctggcaagactagtttttcatttggaagccaacagaccaatagcacagtgcccccatctgccccaccaccaactacagctgccactccccttccaacatcattccccacattgtcatttggtagcctcctgagttcagcaactaccccctccctgcctatgtccgctggcagaagcacagaagaggccacttcatcagctttgcctgagaagccaggtgacagtgaggtctcagcatcagcagcctcacttctagaggagcaacagtcagcccagcttccccaggctcctccgcaaacttctgactctgttaaaaaagaacctgttcttgcccagcctgcagtcagcaactctggcactgcagcatctagtactagtcttgtagcactttctgcagaggctaccccagccaccacgggggtccctgatgccaggacggaggcagtaccacctgcttcctccttttctgtgcctgggcagactgctgtcacagcagctgctatctcaagtgcaggccctgtggccgtcgaaacatcaagtacccccatagcctccagcaccacgtccattgttgctcccggcccatctgcagaggcagcagcatttggtaccgtcacttctggctcatccgtctttgctcagcctcctgctgccagttctagctcagctttcaaccagctcaccaacaacacagccactgccccctctgccacgcccgtgtttgggcaagtggcagccagcaccgcaccaagtctgtttgggcagcagactggtagcacagccagcacagcagctgccacaccacaggtcagcagctcagggtttagcagcccagcttttggtaccacagccccaggggtctttggacagacaaccttcgggcaggcctcagtctttgggcagtcggcgagcagtgctgcaagtgtcttttccttcagtcagcctgggttcagttccgtgcctgccttcggtcagcctgcttcctccactcccacatccaccagtggaagtgtctttggtgccgcctcaagtaccagtagctccagttccttctcatttggacagtcttctcccaacacaggaggggggctgtttggccaaagcaacgctcctgcttttgggcagagtcctggctttggacagggaggctctgtctttggtggtacctcagctgccaccacaacagcagcaacctctgggttcagcttttgccaagcttcaggttttgggtctagtaatactggttctgtgtttggtcaagcagccagtactggtggaatagtctttggccagcaatcatcctcttccagtggtagcgtgtttgggtctggaaacactggaagagggggaggtttcttcagtggccttggaggaaaacccagtcaggatgcagccaacaaaaacccattcagctcggccagtgggggctttggatccacagctacctcaaatacctctaacctatttggaaacagtggggccaagacatttggtggatttgccagctcgtcgtttggagagcagaaacccactggcactttcagctctggaggaggaagtgtggcatcccaaggctttgggttttcctctccaaacaaaacaggtggcttcggtgctgctccagtgtttggcagccctcctacttttgggggatcccctgggtttggaggggtgccagcattcggttcagccccagcctttacaagccctctgggctcgacgggaggcaaagtgttcggagagggcactgcagctgccagcgcaggaggattcgggtttgggagcagcagcaacaccacatccttcggcacgctcgcgagtcagaatgcccccactttcggatcactgtcccaacagacttctggttttgggacccagagtagcggattctctggttttggatcaggcacaggaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccagcggccactcaggacttcagcaagctcctcagttcggtgaaggaaatcagtgacatagtgcagaggtag

SEQ ID NO: 20 - DNA, 1971 bp, Homo sapiens
  source 1..1971: mol_type = unassigned DNA
atgttggagatctgcctgaagctggtgggctgcaaatccaagaaggggctgtcctcgtcctccagctgttatctggaagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgagaagtacatggagtttgacctgaacaatgaaggcgagattgacctgatgtctttaaagaggatgatggagaagcttggtgtccccaagacccacctggagatgaagaagatgatctcagaggtgacaggaggggtcagtgacactatatcctaccgagactttgtgaacatgatgctggggaaacggtcggctgtcctcaagttagtcatgatgtttgaaggaaaagccaacgagagcagccccaagccagttggcccccctccagagagagacattgctagcctgccctga

SEQ ID NO: 21 - DNA, 3114 bp, Homo sapiens
  source 1..3114: mol_type = unassigned DNA
atggaggaaagaccggcagagaccaatgccaatgtggacaactcggcgtccccctcggtggcccagctggccgggcggtttagggagcaggcggctgcagccaaggagacaccagccagtaaaccaacccgaaggaaaccgccctgttccctccccctgttcccccccaaggtagacctgggccagaatggtgaggagaaatcaccacccaatgcgagccaccctcctaaattcaaggtcaagagctcgcctctgattgagaagcttcagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccagcggccactcaggacttcagcaagctcctcagttcggtgaaggaaatcagtgacatagtgcagaggtag

SEQ ID NO: 22 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 23 - protein, 1378 aa, Homo sapiens
MAAADAEAVPARGEPQQDCCVKTELLGEETPMAADEGSAEKQAGEAHMAADGETNGSCENSDASSHANAAKHTQDSARVNPQDGTNTLTRIAENGVSERDSEAAKQNHVTADDFVQTSVIGSNGYILNKPALQAQPLRTTSTLASSLPGHAAKTLPGGAGKGRTPSAFPQTPAAPPATLGEGSADTEDRKLPAPGADVKVHRARKTMPKSVVGLHAASKDPREVREARDHKEPKEEINKNISDFGRQQLLPPFPSLHQSLPQNQCYMATTKSQTEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 24 - protein, 1047 aa, Homo sapiens
MADVINVSVNLEAFSQAISAIQALRSSVSRVFDCLKDGMRNKETLEGREKAFIAHFQDNLHSVNRDLNELERLSNLVGKPSENHPLHNSGLLSLDPVQDKTPLYSQLLQAYKWSNKLQYHAGLASGLLNQQSLKRSANQMGVSAKRRPKAQPTTLVLPPQYVDDVISRIDRMFPEMSIHLSRPNGTSAMLLEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 25 - protein, 1399 aa, Homo sapiens
MWATCCNWFCLDGQPEEVPPPQGARMQAYSNPGYSSFPSPTGLEPSCKSCGAHFANTARKQTCLDCKKNFCMTCSSQVGNGPRLCLLCQRFRATAFQREELMKMKVKDLRDYLSLHDISTEMCREKEELVLLVLGQQPVISQEDRTRASTLSPDFPEQQAFLTQPHSSMVPPTSPNLPSSSAQATSVPPAQVQENQQANGHVSQDQEEPVYLESVARVPAEDETQSIDSEDSFVPGRRASLSDLTDLEDIEGLTVRQLKEILARNFVNYKGCCEKWELMERVTRLYKDQKGLQHLEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 26 - protein, 1419 aa, Homo sapiens
MLLGASLVGVLLFSKLVLKLPWTQVGFSLLFLYLGSGGWRFIRVFIKTIRRDIFGGLVLLKVKAKVRQCLQERRTVPILFASTVRRHPDKTALIFEGTDTHWTFRQLDEYSSSVANFLQARGLASGDVAAIFMENRNEFVGLWLGMAKLGVEAALINTNLRRDALLHCLTTSRARALVFGSEMASAICEVHASLDPSLSLFCSGSWEPGAVPPSTEHLDPLLKDAPKHLPSCPDKGFTDKLFYIYTSGTTGLPKAAIVVHSRYYRMAALVYYGFRMRPNDIVYDCLPLYHSAGNIVGIGQCLLHGMTVVIRKKFSASRFWDDCIKYNCTIVQYIGELCRYLLNQPPREAENQHQVRMALGNGLRQSIWTNFSSRFHIPQVAEFYGATECNCSLGNFDSQVGACGFNSRILSFVYPIRLVRVNEDTMELIRGPDGVCIPCQPGEPGQLVGRIIQKDPLRRFDGYLNQGANNKKIAKDVFKKGDQAYLTGDVLVMDELGYLYFRDRTGDTFRWKGENVSTTEVEGTLSRLLDMADVAVYGVEVPGTEGRAGMAAVASPTGNCDLERFAQVLEKELPLYARPIFLRLLPELHKTGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 27 - protein, 2006 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGMMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDGEGAFHGDADGSFGTPPGYGCAADRAEEQRRHQDGLPYIDDSPSSSPHLSSKGRGSRDALVSGALESTKASELDLEKGLEMRKWVLSGILASEETYLSHLEALLLPMKPLKAAATTSQPVLTSQQIETIFFKVPELYEIHKEFYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTDLLLCTKLKKQSGGKTQQYDCKWYIPLTDLSFQMVDELEAVPNIPLVPDEELDALKIKISQIKNDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 28 - protein, 2031 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGMMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDGEGAFHGDADGSFGTPPGYGCAADRAEEQRRHQDGLPYIDDSPSSSPHLSSKGRGSRDALVSGALESTKASELDLEKGLEMRKWVLSGILASEETYLSHLEALLLPMKPLKAAATTSQPVLTSQQIETIFFKVPELYEIHKEFYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTDLLLCTKLKKQSGGKTQQYDCKWYIPLTDLSFQMVDELEAVPNIPLVPDEELDALKIKISQIKNDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEDDESPGLYGFLNVIVHSATGFKQSSKALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 29 - protein, 2031 aa, Homo sapiens; identical to SEQ ID NO: 28

SEQ ID NO: 30 - protein, 2031 aa, Homo sapiens; identical to SEQ ID NO: 28

SEQ ID NO: 31 - protein, 2031 aa, Homo sapiens; identical to SEQ ID NO: 28

SEQ ID NO: 32 - protein, 2031 aa, Homo sapiens; identical to SEQ ID NO: 28

SEQ ID NO: 33 - protein, 2031 aa, Homo sapiens; identical to SEQ ID NO: 28

SEQ ID NO: 34 - protein, 2006 aa, Homo sapiens; identical to SEQ ID NO: 27

SEQ ID NO: 35 - protein, 2031 aa, Homo sapiens; identical to SEQ ID NO: 28

SEQ ID NO: 36 - protein, 2031 aa, Homo sapiens; identical to SEQ ID NO: 28

SEQ ID NO: 37 - protein, 2031 aa, Homo sapiens; identical to SEQ ID NO: 28

SEQ ID NO: 38 - protein, 2031 aa, Homo sapiens; identical to SEQ ID NO: 28

SEQ ID NO: 39 - protein, 2031 aa, Homo sapiens; identical to SEQ ID NO: 28

SEQ ID NO: 40 - protein, 3117 aa, Homo sapiens
MGDEMDAMIPEREMKDFQFRALKKVRIFDSPEELPKERSSLLAVSNKYGLVFAGGASGLQIFPTKNLLIQNKPGDDPNKIVDKVQGLLVPMKFPIHHLALSCDNLTLSACMMSSEYGSIIAFFDVRTFSNEAKQQKRPFAYHKLLKDAGGMVIDMKWNPTVPSMVAVCLADGSIAVLQVTETVKVCATLPSTVAVTSVCWSPKGKQLAVGKQNGTVVQYLPTLQEKKVIPCPPFYESDHPVRVLDVLWIGTYVFAIVYAAADGTLETSPDVVMALLPKKEEKHPEIFVNFMEPCYGSCTERQHHYYLSYIEEWDLVLAASAASTEVSILARQSDQINWESWLLEDSSRAELPVTDKSDDSLPMGVVVDYTNQVEITISDEKTLPPAPVLMLLSTDGVLCPFYMINQNPGVKSLIKTPERLSLEGERQPKSPGSTPTTPTSSQAPQKLDASAAAAPASLPPSSPAAPIATFSLLPAGGAPTVFSFGSSSLKSSATVTGEPPSYSSGSDSSKAAPGPGPSTFSFVPPSKASLAPTPAASPVAPSAASFSFGSSGFKPTLESTPVPSVSAPNIAMKPSFPPSTSAVKVNLSEKFTAAATSTPVSSSQSAPPMSPFSSASKPAASGPLSHPTPLSAPPSSVPLKSSVLPSPSGRSAQGSSSPVPSMVQKSPRITPPAAKPGSPQAKSLQPAVAEKQGHQWKDSDPVMAGIGEEIAHFQKELEELKARTSKACFQVGTSEEMKMLRTESDDLHTFLLEIKETTESLHGDISSLKTTLLEGFAGVEEAREQNERNRDSGYLHLLYKRPLDPKSEAQLQEIRRLHQYVKFAVQDVNDVLDLEWDQHLEQKKKQRHLLVPERETLFNTLANNREIINQQRKRLNHLVDSLQQLRLYKQTSLWSLSSAVPSQSSIHSFDSDLESLCNALLKTTIESHTKSLPKVPAKLSPMKQAQLRNFLAKRKTPPVRSTAPASLSRSAFLSQRYYEDLDEVSSTSSVSQSLESEDARTSCKDDEAVVQAPRHAPVVRTPSIQPSLLPHAAPFAKSHLVHGSSPGVMGTSVATSASKIIPQGADSTMLATKTVKHGAPSPSHPISAPQAAAAAALRRQMASQAPAVNTLTESTLKNVPQVVNVQELKNNPATPSTAMGSSVPYSTAKTPHPVLTPVAANQAKQGSLINSLKPSGPTPASGQLSSGDKASGTAKIETAVTSTPSASGQFSKPFSFSPSGTGFNFGIITPTPSSNFTAAQGATPSTKESSQPDAFSSGGGSKPSYEAIPESSPPSGITSASNTTPGEPAASSSRPVAPSGTALSTTSSKLETPPSKLGELLFPSSLAGETLGSFSGLRVGQADDSTKPTNKASSTSLTSTQPTKTSGVPSGFNFTAPPVLGKHTEPPVTSSATTTSVAPPAATSTSSTAVFGSLPVTSAGSSGVISFGGTSLSAGKTSFSFGSQQTNSTVPPSAPPPTTAATPLPTSFPTLSFGSLLSSATTPSLPMSAGRSTEEATSSALPEKPGDSEVSASAASLLEEQQSAQLPQAPPQTSDSVKKEPVLAQPAVSNSGTAASSTSLVALSAEATPATTGVPDARTEAVPPASSFSVPGQTAVTAAAISSAGPVAVETSSTPIASSTTSIVAPGPSAEAAAFGTVTSGSSVFAQPPAASSSSAFNQLTNNTATAPSATPVFGQVAASTAPSLFGQQTGSTASTAAATPQVSSSGFSSPAFGTTAPGVFGQTTFGQASVFGQSASSAASVFSFSQPGFSSVPAFGQPASSTPTSTSGSVFGAASSTSSSSSFSFGQSSPNTGGGLFGQSNAPAFGQSPGFGQGGSVFGGTSAATTTAATSGFSFCQASGFGSSNTGSVFGQAASTGGIVFGQQSSSSSGSVFGSGNTGRGGGFFSGLGGKPSQDAANKNPFSSASGGFGSTATSNTSNLFGNSGAKTFGGFASSSFGEQKPTGTFSSGGGSVASQGFGFSSPNKTGGFGAAPVFGSPPTFGGSPGFGGVPAFGSAPAFTSPLGSTGGKVFGEGTAAASAGGFGFGSSSNTTSFGTLASQNAPTFGSLSQQTSGFGTQSSGFSGFGSGTGGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 41 - protein, 656 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESEKYMEFDLNNEGEIDLMSLKRMMEKLGVPKTHLEMKKMISEVTGGVSDTISYRDFVNMMLGKRSAVLKLVMMFEGKANESSPKPVGPPPERDIASLP

SEQ ID NO: 42 - protein, 1037 aa, Homo sapiens
MEERPAETNANVDNSASPSVAQLAGRFREQAAAAKETPASKPTRRKPPCSLPLFPPKVDLGQNGEEKSPPNASHPPKFKVKSSPLIEKLQLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 43 - DNA, 8012 bp, Homo sapiens
  source 1..8012: mol_type = unassigned DNA
agctgcagcggagccgcggagcgggcggcggggcccaggctgtgcgcttggggagcgcggaatgtgaggcttggcgggccgcagcacgctcggacgggccaggggcggcgacccctcgcggacgcccggctgcgcgccgggccggggacttgcccttgcacgctccctgcgccctccagctcgccggcgggaccatgaagaagttctctcggatgcccaagtcggagggcggcagcggcggcggagcggcgggtggcggggctggcggggccggggccggggccggctgcggctccggcggctcgtccgtgggggtccgggtgttcgcggtcggccgccaccaggtcaccctggaagagtcgctggccgaaggtggattctccacagttttcctcgtgcgtactcacggtggaatccgatgtgcattgaagcgaatgtatgtcaataacatgccagacctcaatgtttgtaaaagggaaattacaattatgaaagagctatctggtcacaaaaatattgtgggctatttggactgtgctgttaattcaattagtgataatgtatgggaagtccttatcttaatggaatattgtcgagctggacaggtagtgaatcaaatgaataagaagctacagacgggttttacagaaccagaagtgttacagatattctgtgatacctgtgaagctgttgcaaggttgcatcagtgtaagactccaataattcaccgggatctgaaggtagaaaatattttgttgaatgatggtgggaactatgtactttgtgactttggcagtgccactaataaatttcttaatcctcaaaaagatggagttaatgtagtagaagaagaaattaaaaagtatacaactctgtcatacagagcccctgaaatgatcaacctttatggagggaaacccatcaccaccaaggctgatatctgggcactgggatgtctactctataaactttgtttcttcactcttccttttggtgagagtcaggttgctatctgtgatggcaacttcaccatcccagacaattctcgttactcccgtaacatacattgcttaataaggttcatgcttgaaccagatccggaacatagacctgatatatttcaagtgtcatattttgcatttaaatttgccaaaaaggattgtccagtctccaacatcaataattcttctattccttcagctcttcctgaaccgatgactgctagtgaagcagctgctaggaaaagccaaataaaagccagaataacagataccattggaccaacagaaacctcaattgcaccaagacaaagaccaaaggccaactctgctactactgccactcccagtgtgctgaccattcaaagttcagcaacacctgttaaagtccttgctcctggtgaattcggtaaccatagaccaaaaggggcactaagacctggaaatggccctgaaattttattgggtcagggacctcctcagcagccgccacagcagcatagagtactccagcaactacagcagggagattggagattacagcaactccatttacagcatcgtcatcctcaccagcagcagcagcagcagcagcagcaacagcaacagcagcagcagcaacagcaacagcagcagcagcagcagcagcagcagcaccaccaccaccaccaccaccacctacttcaagatgcttatatgcagcagtatcaacatgcaacacagcagcaacagatgcttcaacaacaatttttaatgcattcggtatatcaaccacaaccttctgcatcacagtatcctacaatgatgccgcagtatcagcaggctttctttcaacagcagatgctagctcaacatcagccgtctcaacaacaggcatcacctgaatatcttacctcccctcaagagttctcaccagccttagtttcctacacttcatcacttccagctcaggttggaaccataatggactcctcctatagtgccaataggtcagttgctgataaagaggccattgcaaatttcacaaatcagaagaacatcagcaatccacctgatatgtcagggtggaatccttttggagaggataatttctctaagttaacagaagaggaactattggacagagaatttgaccttctaagatcaaataggctcgaggagagagcatcctcagataagaatgtagactcactttctgctccacataaccatcctccagaagatccttttggttctgttcctttcatttctcattcaggttctcctgaaaagaaagctgaacattcatctataaatcaagaaaatggcactgcaaaccctatcaagaacggtaaaacaagtccagcatctaaagatcagcggactggaaagaaaacctcagtacagggtcaagtgcaaaaggggaatgatgaatctgaaagtgattttgaatcagatcccccttctcctaagagcagtgaagaggaagagcaagatgatgaagaagttcttcagggggaacaaggagattttaatgatgatgatactgaaccagaaaatctgggtcataggcctctcctcatggattctgaagatgaggaagaagaggagaaacatagctctgattctgattatgagcaggctaaagcaaagtacagtgacatgagctctgtctacagagacagatctggcagtggaccaacccaagatcttaatacaatactcctcacctcagcccaattatcctctgatgttgcagtggagactcccaaacaggagtttgatgtatttggcgctgtccccttctttgcagtgcgtgctcaacagccccagcaagaaaagaatgaaaagaacctccctcaacacaggtttcctgctgcaggactggagcaggaggaatttgatgtattcacaaaggcgccttttagcaagaaggtgaatgtacaagaatgccatgcagtggggcctgaggcacatactatccctggttatcccaaaagtgtagatgtatttggctccactccatttcagcccttcctcacatcaacaagtaaaagtgaaagcaatgaggacctttttgggcttgtgccctttgatgaaataacggggagccagcagcaaaaagtcaaacagcgcagcttacagaaactgtcctctcgccaaaggcgcacaaagcaggatatgtccaaaagtaatgggaagcggcatcatggcacgccaactagcacaaagaagactttgaagcctacctatcgcactccagagagggctcgcaggcacaaaaaagtgggccgccgagactctcaaagtagcaatgaatttttaaccatctcagactccaaggagaacattagtgttgcactgactgatgggaaagatagggggaatgtcttacaacctgaggagagcctgttggaccccttcggtgccaagcccttccattctccagacctgtcatggcaccctccacatcagggcctgagcgacatccgtgctgatcacaatactgtcctgccagggcggccaagacaaaattcactacatgggtcattccatagtgcagatgtattgaaaatggatgattttggtgccgtgccctttacagaacttgtggtgcaaagcatcactccacatcagtcccaacagtcccaaccagtcgaattagacccatttggtgctgctccatttccttctaaacagtagatacttctgatggattctcggcattaactcctgtttcaaaaaagtgtgaacagttttatgaatttgaaagaaaatttggtagctctttatagcattcattcttaaagatcagtcagaataggtgatttctaaataaaccaaatagaagaatgaagtatctctacagggtagtaacttgattcctcttcaggagaaaagggagctaaattgcaagctctaactaagggtttctgctactgacatcacaacacagaaatgcaagtgtggtacttccagtgaaagcacatggcacctttctaggtgtgtagccactgagaagggacagtgaaactgttatttttgatatcagaatgtcatttttatgtgcatatccctaaaattagggttatttctacatacactagttacacttgtgaattttttttaaggtctcttttaatttccagacagttaaaaacaatctagttatcttaaagcattagaaagttattatctggagagtgcagagatttcagtccatacacctttctccacaaagcagagccagaagtaactgactattgtgcctaaaactctgtttcatttttaaaaacaagtgccattaaaatggaatatctaatgataagcatatgaaataatgtgtaattagctcaatttaactattccacaacttacatattccaaaacaatgttatacatgataaatatatataatttttgtcagttaaaacaaattaaaaaaatggactatcgtcgcacagaagcctagaacaaaaatatgaagagaaatatctgacatttgtaaagaaattataagaagaaaaaaagatacagaacagaaaacattcactactttagaaacactttatgcatggcttcttgccccaaacttttattgtgatggccctaataaagcagattattggaaaaattggaggacaagggttgtataaaaattttattttatgaagaaaatatgtagcggaaactgaattttcaagacatttacaatgtgaaatcatgttgcatttaacaatgtactttattagcaacttcaccaaatattccccaagtcataagcaacaattatttttattaggttttggggggtggagtagttttaataaagtgcacagaatggtgacacccacaaagccttatataaaggcaggattcatgcatcctgctgcaagtacctctgcactaatataccagatcctaaaatgcatataaggtggactagcatcttaattctgctagttgattgtgtctttactgaaaagaacccagctaccaatttgcctttttttacaccacaaatcctaattagaaacttgaggttttatagaaatcatttaatgatagagattacatatgtgaattaatgtgaatatagtatctgtgcttcctgtgtctatgactattttaagatataattgtgctgcgctatcagattaacatttggaagtttctagaacagttaatgctatttacagaaaggagtagaaactcatcaactggcactctctttgatttttatattttaaattaactctcttcgatctcaaagtatattttacgagtaattttattaggaatctcttatagtgccccaatgggataagctatttgcctattttcacagttctgaacttggaaagaagcaaagtatatgtaactaaaccacatatttgtctttttattgctttttcccttcttttatatgctaaatcaaatatagatttgtggatagggaagcaatatgtgaatcacaatgtagcagaggcagaccaagcattacattattatttagagctggactgcaccaattacttgtcctcgtgccaaaggcaaatatgtttgcacctttttttttttttctgattctcaggttgattaatactgctaatgcaaatgctcaagtagatgtttaaaaactttacaaaatagattcaagtgatactttcttttaaaagtgaagagttgatgattacacatagtaaattcatgaactacagtaggtttgtatcaaacaattttttttaatgaaaatctgttgaggtgtacacaatatgcttcttgattgtattagtccttggtctctgctagacctcatgagtttcatcatttagaaaaggggtagaggatgaactaatgtctccttcagatgtaaacatgaaatacctagagttttacttgcttttcaatacactgaataattttaaatgattctgacactgatgtagaccctttgacttataaattctgaggaaacaactgacagcataaaatatttacattcttataacacagcacagtgactttcttctttcaagattgtagctcagagaaaagatacaggattcaattgggggttcaataggatagaaatggagagattcctttgtgttgtagtagaggcattttcctaaggagtatagatttatactttgcattttcattcatcatcccccagaatcatggtcaaggtgtaggtcactccacacagctgatgctcaggttattcccttgtgagaattatgagaataaagctcccaagatatgtgaaagtgcttaacacagtacctggcacacagcactcaataaaagtttggctctattatgggatggttcaattctggtttaaggaaggaagaaaggttattatatatgtaccactaagcaaatatatatatatatatatatttggggttttttttccctaatattatttgggtgtcccctgtgcttctttaggatgtagttataactaaacctgttatacttgaacatcactaagagaagtaaattattatgaagctagcaaaaatcttgaggccaaagttgtttcttaacagctttaataatgcttgttgattttgaataatcctttaaaaagtggaccatttgcttattttaatatcacgtcagtaaaatgttagtattaaaaagatcagctttttatggcattgaagaatgtatctgctaagacacaaaaattgcatggtaagtataataggtggaggaggaaaggttgtaggccggatgaaaatttaactgactagaacatttattcaggagtgtaattattttcccttaccccaatccctgtgtacgtgttgggtatagttacgacattatccggatttgcaaatagacacaactttcagtcttaccctgtttattgtttaagagtgatagactgttgtcctcttgctggtggaaaatctaaggtggagcccactcttctatgctgaagttcaccaggcagagcagttttcttacaagtcagctactctgcttggtttattttaggttttggtacttcacgtaagcactgttagaaggtacaagtgtattaatatcactagttttgaggcgcttgggtacatttgtttttaatatatttagaatgtgcagtaaacttttttctcatttttttttctttttagcaaacttgttattttaggtccaattattgagttgacagtctactgtgagaatgagatgacatatctactgtgagaataccataaatgatgaatagtttatttgagaacttttatactcagtggtgttttatatattaagataaaaatatgtacacacatgcatgtcacatctctctactgtggagttaatgtgaatttttaaaaaatggaattgcaaccacaatcatatctaagagaacattcactcctagtgagggtttacaaaagctactaagagaataaggtagatgataatgcaaagggtcatgatttggggttatttttgttttattttaaaatttatactgctacttttgaaagaattgtttttatgactatgctctttttgtgattgaaaagtcatctaatagaagctgtatagaagctactttttaattgctggcaaacagctttaagtgcactttctttgattacacttccattttttgttaaacttgaattttctgaagccttttatgtaccactaagcaaataactttaacctttaaataaagcaaatttacatctttattgtatttctacttgttacaaaacatacttgctaaagtaacttcagtcctcaaatatagctgggaaacaattatgagatagactcagtctccccctcccaccccttttcccctgccatatctaattaagcactaactgattttattactttattgcctttacactgcttattctttttgactgaattctgtccctgattcactgttttgtttgaaatttaaagttattttcttactgtatttatcatacctgttttaatctgttttctttaaatgcaataaatcccaaatggattgcatattctttataa

SEQ ID NO: 44 - protein, 1161 aa, Homo sapiens
MKKFSRMPKSEGGSGGGAAGGGAGGAGAGAGCGSGGSSVGVRVFAVGRHQVTLEESLAEGGFSTVFLVRTHGGIRCALKRMYVNNMPDLNVCKREITIMKELSGHKNIVGYLDCAVNSISDNVWEVLILMEYCRAGQVVNQMNKKLQTGFTEPEVLQIFCDTCEAVARLHQCKTPIIHRDLKVENILLNDGGNYVLCDFGSATNKFLNPQKDGVNVVEEEIKKYTTLSYRAPEMINLYGGKPITTKADIWALGCLLYKLCFFTLPFGESQVAICDGNFTIPDNSRYSRNIHCLIRFMLEPDPEHRPDIFQVSYFAFKFAKKDCPVSNINNSSIPSALPEPMTASEAAARKSQIKARITDTIGPTETSIAPRQRPKANSATTATPSVLTIQSSATPVKVLAPGEFGNHRPKGALRPGNGPEILLGQGPPQQPPQQHRVLQQLQQGDWRLQQLHLQHRHPHQQQQQQQQQQQQQQQQQQQQQQQQQQQHHHHHHHHLLQDAYMQQYQHATQQQQMLQQQFLMHSVYQPQPSASQYPTMMPQYQQAFFQQQMLAQHQPSQQQASPEYLTSPQEFSPALVSYTSSLPAQVGTIMDSSYSANRSVADKEAIANFTNQKNISNPPDMSGWNPFGEDNFSKLTEEELLDREFDLLRSNRLEERASSDKNVDSLSAPHNHPPEDPFGSVPFISHSGSPEKKAEHSSINQENGTANPIKNGKTSPASKDQRTGKKTSVQGQVQKGNDESESDFESDPPSPKSSEEEEQDDEEVLQGEQGDFNDDDTEPENLGHRPLLMDSEDEEEEEKHSSDSDYEQAKAKYSDMSSVYRDRSGSGPTQDLNTILLTSAQLSSDVAVETPKQEFDVFGAVPFFAVRAQQPQQEKNEKNLPQHRFPAAGLEQEEFDVFTKAPFSKKVNVQECHAVGPEAHTIPGYPKSVDVFGSTPFQPFLTSTSKSESNEDLFGLVPFDEITGSQQQKVKQRSLQKLSSRQRRTKQDMSKSNGKRHHGTPTSTKKTLKPTYRTPERARRHKKVGRRDSQSSNEFLTISDSKENISVALTDGKDRGNVLQPEESLLDPFGAKPFHSPDLSWHPPHQGLSDIRADHNTVLPGRPRQNSLHGSFHSADVLKMDDFGAVPFTELVVQSITPHQSQQSQPVELDPFGAAPFPSKQ

SEQ ID NO: 45 - DNA, 5095 bp, Homo sapiens
  source 1..5095: mol_type = unassigned DNA
ggggccacgctgcgggcccgggccatggccgccgccgatgccgaggcagttccggcgaggggggagcctcagcaggattgctgtgtgaaaaccgagctgctgggagaagagacacctatggctgccgatgaaggctcagcagagaaacaggcaggagaggcccacatggctgcggacggtgagaccaatgggtcttgtgaaaacagcgatgccagcagtcatgcaaatgctgcaaagcacactcaggacagcgcaagggtcaacccccaggatggcaccaacacactaactcggatagcggaaaatggggtttcagaaagagactcagaagcggcgaagcaaaaccacgtcactgccgacgactttgtgcagacttctgtcatcggcagcaacggatacatcttaaataagccggccctacaggcacagcccttgaggactaccagcactctggcctcttcgctgcctggccatgctgcaaaaacccttcctggaggggctggcaaaggcaggactccaagcgcttttccccagacgccagccgccccaccagccacccttggggaggggagtgctgacacagaggacaggaagctcccggcccctggcgccgacgtcaaggtccacagggcacgcaagaccatgccgaagtccgtcgtgggcctgcatgcagccagtaaagatcccagagaagttcgagaagctagagatcataaggaaccaaaagaggagatcaacaaaaacatttctgactttggacgacagcagcttttaccccccttcccatcccttcatcagtcgctacctcagaaccagtgctacatggccaccacaaaatcacagacagcttgcttgccttttgttttagcagctgcagtatctcggaagaaaaaacgaagaatgggaacctatagcctggttcctaagaaaaagaccaaagtattaaaacagaggacggtgattgagatgtttaagagcataactcattccactgtgggttccaagggggagaaggacctgggcgccagcagcctgcacgtgaatggggagagcctggagatggactcggatgaggacgactcagaggagctcgaggaggacgacggccatggtgcagagcaggcggccgcgttccccacagaggacagcaggacttccaaggagagcatgtcggaggctgatcgcgcccagaagatggacggggagtccgaggaggagcaggagtccgtggacaccggggaggaggaggaaggcggtgacgagtctgacctgagttcggaatccagcattaagaagaaatttctcaagaggaaaggaaagaccgacagtccctggatcaagccagccaggaaaaggaggcggagaagtagaaagaagcccagcggtgccctcggttctgagtcgtataagtcatctgcaggaagcgctgagcagacggcaccaggagacagcacagggtacatggaagtttctctggactccctggatctccgagtcaaaggaattctgtcttcacaagcagaagggttggccaacggtccagatgtgctggagacagacggcctccaggaagtgcctctctgcagctgccggatggaaacaccgaagagtcgagagatcaccacactggccaacaaccagtgcatggctacagagagcgtggaccatgaattgggccggtgcacaaacagcgtggtcaagtatgagctgatgcgcccctccaacaaggccccgctcctcgtgctgtgtgaagaccaccggggccgcatggtgaagcaccagtgctgtcctggctgtggctacttctgcacagcgggtaattttatggagtgtcagcccgagagcagcatctctcaccgtttccacaaagactgtgcctctcgagtcaataacgccagctattgtccccactgtggggaggagagctccaaggccaaagaggtgacgatagctaaagcagacaccacctcgaccgtgacaccagtccccgggcaggagaagggctcggccctggagggcagggccgacaccacaacgggcagtgctgccgggccaccactctcggaggacgacaagctgcagggtgcagcctcccacgtgcccgagggctttgatccaacgggacctgctgggcttgggaggccaactcccggcctttcccagggaccagggaaggaaaccttggagagcgctctcatcgccctcgactcggaaaaacccaagaagcttcgcttccacccaaagcagctgtacttctccgccaggcaaggggagcttcagaaggtgctcctcatgctggtggacggaattgaccccaacttcaaaatggagcaccagaataagcgctctccactgcacgccgcggcagaggctggacacgtggacatctgccacatgctggttcaggcgggcgctaatattgacacctgctcagaagaccagaggaccccgttgatggaagcagccgaaaacaaccatctggaagcagtgaagtacctcatcaaggctggggccctggtggatcccaaggacgcagagggctctacgtgtttgcacctggctgccaagaaaggccactacgaagtggtccagtacctgctttcaaatggacagatggacgtcaactgtcaggatgacggaggctggacacccatgatctgggccacagagtacaagcacgtggacctcgtgaagctgctgctgtccaagggctctgacatcaacatccgagacaacgaggagaacatttgcctgcactgggcggcgttctccggctgcgtggacatagccgagatcctgctggctgccaagtgcgacctccacgccgtgaacatccacggagactcgccactgcacattgccgcccgggagaaccgctacgactgtgtcgtcctctttctttctcgggattcagatgtcaccttaaagaacaaggaaggagagacgcccctgcagtgtgcgagcctcaactctcaggtgtggagcgctctgcagatgagcaaggctctgcaggactcggcccccgacaggcccagccccgtggagaggatagtgagcagggacatcgctcgaggctacgagcgcatccccatcccctgtgtcaacgccgtggacagcgagccatgccccagcaactacaagtacgtctctcagaactgcgtgacgtcccccatgaacatcgacagaaatatcactcatctgcagtactgcgtgtgcatcgacgactgctcctccagcaactgcatgtgcggccagctcagcatgcgctgctggtacgacaaggatggccggctcctgccagagttcaacatggcggagcctcccttgatcttcgaatgcaaccacgcgtgctcctgctggaggaactgccgaaatcgcgtcgtacagaatggtctcagggcaaggctgcagctctaccggacgcgggacatgggctggggcgtgcggtccctgcaggacatcccaccaggcacctttgtctgcgagtatgttggggagctgatttcagactcagaagccgacgttcgagaggaagattcttacctctttgatctcgacaataaggacggggaggtttactgcatcgacgcgcggttctacgggaacgtcagccggttcatcaaccaccactgcgagcccaacctggtgcccgtgcgcgtgttcatggcccaccaggacctgcggttcccccggatcgccttcttcagcacccgcctgatcgaggccggcgagcagctcgggtttgactatggagagcgcttctgggacatcaaaggcaagctcttcagctgccgctgcggctcccccaagtgccggcactcgagcgcggccctggcccagcgtcaggccagcgcggcccaggaggcccaggaggacggcttgcccgacaccagctccgcggctgccgccgaccccctatgagacgccgccggccagcggggcgctcgggagccagggaccgccgcgtcgccgattagaggacgaggaggagagattccgcacgcaaccgaaagggtccttcggggctgcgccgccggcttcctggaggggtcggaggtgaggctgcagcccctgcgggcgggtgtggatgcctcccagccaccttcccagacctgcggcctcaccgcgggcccagtgcccaggctggagcgcacactttggtccgcgcgccagagacgctgggagtccgcactggcatcaccttctgagtttctgatgctgatttgtcgttgcgaagtttctcgtttcttcctctgacctccgaggtccccgctgcaccacggggttgctctgttctcctgtccggcccagactcttctgtgtggcgccgccgaagccaccgttagcgcgagctgctccgttcgccctgcccacggcctgcgtggctggggccgagtcccaggggccgcacggagggcacagtctcctgtcaggctcggagaggtcaggagaccgaccccaccactaactttggagaaaatgtgggtttgctttttaaaggaatcctatatctagtcctatatatcaaacctctaactgacgtttcttttcgaggaagtggcttggtgggtgcagcccccgccggttccgttgacgctggcaccttctgttgattttttaagccacatgctatgatgaataaactgatttattttctaccattactgaacattaggacaaacacaaaataaaaaacaaaacacagacaacggtgctgattctggtgtggtttctactcaccacgtgaaataaactatcaactgtataaagagaacaaagtgattttagaataaaatgcaggaaaaacttttttaaagatgttagtcttgtagcgtgaataaatttgccatcaccttttgtgtggtggcctggcaggtcatatacttttttttggcatatacctttttaaagactgtaattagtgcagtaacagtggggttttttttgtgcaactcttctaaaaacattcataatgcagtcatgtttatttttttctgttaaaatgtttttgacagttttaagagcagtcttttggctctgaccatttcttgttctgtttccaatgaaatcaataaaaaaaaagaagtactttaaa

SEQ ID NO: 46 - protein, 1298 aa, Homo sapiens
MAAADAEAVPARGEPQQDCCVKTELLGEETPMAADEGSAEKQAGEAHMAADGETNGSCENSDASSHANAAKHTQDSARVNPQDGTNTLTRIAENGVSERDSEAAKQNHVTADDFVQTSVIGSNGYILNKPALQAQPLRTTSTLASSLPGHAAKTLPGGAGKGRTPSAFPQTPAAPPATLGEGSADTEDRKLPAPGADVKVHRARKTMPKSVVGLHAASKDPREVREARDHKEPKEEINKNISDFGRQQLLPPFPSLHQSLPQNQCYMATTKSQTACLPFVLAAAVSRKKKRRMGTYSLVPKKKTKVLKQRTVIEMFKSITHSTVGSKGEKDLGASSLHVNGESLEMDSDEDDSEELEEDDGHGAEQAAAFPTEDSRTSKESMSEADRAQKMDGESEEEQESVDTGEEEEGGDESDLSSESSIKKKFLKRKGKTDSPWIKPARKRRRRSRKKPSGALGSESYKSSAGSAEQTAPGDSTGYMEVSLDSLDLRVKGILSSQAEGLANGPDVLETDGLQEVPLCSCRMETPKSREITTLANNQCMATESVDHELGRCTNSVVKYELMRPSNKAPLLVLCEDHRGRMVKHQCCPGCGYFCTAGNFMECQPESSISHRFHKDCASRVNNASYCPHCGEESSKAKEVTIAKADTTSTVTPVPGQEKGSALEGRADTTTGSAAGPPLSEDDKLQGAASHVPEGFDPTGPAGLGRPTPGLSQGPGKETLESALIALDSEKPKKLRFHPKQLYFSARQGELQKVLLMLVDGIDPNFKMEHQNKRSPLHAAAEAGHVDICHMLVQAGANIDTCSEDQRTPLMEAAENNHLEAVKYLIKAGALVDPKDAEGSTCLHLAAKKGHYEVVQYLLSNGQMDVNCQDDGGWTPMIWATEYKHVDLVKLLLSKGSDINIRDNEENICLHWAAFSGCVDIAEILLAAKCDLHAVNIHGDSPLHIAARENRYDCVVLFLSRDSDVTLKNKEGETPLQCASLNSQVWSALQMSKALQDSAPDRPSPVERIVSRDIARGYERIPIPCVNAVDSEPCPSNYKYVSQNCVTSPMNIDRNITHLQYCVCIDDCSSSNCMCGQLSMRCWYDKDGRLLPEFNMAEPPLIFECNHACSCWRNCRNRVVQNGLRARLQLYRTRDMGWGVRSLQDIPPGTFVCEYVGELISDSEADVREEDSYLFDLDNKDGEVYCIDARFYGNVSRFINHHCEPNLVPVRVFMAHQDLRFPRIAFFSTRLIEAGEQLGFDYGERFWDIKGKLFSCRCGSPKCRHSSAALAQRQASAAQEAQEDGLPDTSSAAAADPL

SEQ ID NO: 47 - DNA, 1385 bp, Homo sapiens
  source 1..1385: mol_type = unassigned DNA
agagctgctgtggcggcggcaacatggcggacgtgataaatgtcagtgtgaacctggaggccttttcccaggccattagtgccatccaggcgctgcgctccagcgtgagcagggtgttcgactgcctgaaggatgggatgcggaacaaggagacgctggagggccgggagaaggcctttattgcgcacttccaggacaacttacattcggtcaaccgggacctcaatgagctggaacgtctgagcaatctggtaggcaagccatctgagaaccatcctcttcataacagtgggctgttaagcctggatcctgtgcaggacaaaactcctctctatagtcaactccttcaagcatataagtggtcaaacaagttgcagtaccatgcaggactagcatctggccttttaaatcagcagtcattgaagcgttccgctaatcagatgggagtatctgccaaacgtagaccaaaggctcagcccacaactcttgtcctaccacctcaatatgttgatgatgtgatcagccgcattgacaggatgtttcctgaaatgtccatccacttatccagacccaatggaacatcagcaatgcttctggtgaccttgggaaaggtgttgaaagtgatcgtcgtcatgcggagcctgttcattgatcgaacaatagtaaagggatataacgagaatgtctacacagaagatggcaagcttgatatatggtccaaatccaactatcaagtattccagaaggtgacagaccatgccaccactgccctgctccactatcagctgccccagatgccggatgtcgtggtccgatccttcatgacctggttaagaagttacataaagctgttccaggccccgtgccagcgctgcgggaagtttctgcaggacggccttcccccgacatggagggatttccgaaccctcgaagccttccatgacacctgccggcagtagcccccacgctggccccagcctcagaccccacccagcaccttcccagacacgcaggaagcccacagaaggctcagctggttcctcactgcccagatgtgtacagctgctcctccctttcataaagcagcgccatgtgtgcagaggccactcttgaagagcagactccctctgtggctgatgggactaattattcccactagccagcggactgaaggcaaagaagacctttctagaacctggtagaaggaagctgtgcagcatgctcctcgtccatgtgtgtcggcagtgctggtgtctgtcgtctccgcgagctgttactggaatgagcccttgtgttcatgggtatcgtcatgcggggttcttgtgttttgtggggcttgggttttggtaacttatttttataagcaataaaccttttgtatcctg

SEQ ID NO: 48 - protein, 311 aa, Homo sapiens
MADVINVSVNLEAFSQAISAIQALRSSVSRVFDCLKDGMRNKETLEGREKAFIAHFQDNLHSVNRDLNELERLSNLVGKPSENHPLHNSGLLSLDPVQDKTPLYSQLLQAYKWSNKLQYHAGLASGLLNQQSLKRSANQMGVSAKRRPKAQPTTLVLPPQYVDDVISRIDRMFPEMSIHLSRPNGTSAMLLVTLGKVLKVIVVMRSLFIDRTIVKGYNENVYTEDGKLDIWSKSNYQVFQKVTDHATTALLHYQLPQMPDVVVRSFMTWLRSYIKLFQAPCQRCGKFLQDGLPPTWRDFRTLEAFHDTCRQ

SEQ ID NO: 49 - DNA, 7210 bp, Homo sapiens
  source 1..7210: mol_type = unassigned DNA
acttcacatgaaagccgctgctcttggttcctgagatgatctcatctgaacatggctccagcgacctcagccacaggaactagagacagcttgctttctgtcgtggcaatgttttgaaactggcaaaatcagattttatcatgtgggcaacctgctgcaactggttctgcctggatggacagcctgaggaggtcccaccaccccagggagccaggatgcaggcctattccaaccctgggtacagctccttcccttccccaacaggcttggaaccaagctgcaagtcctgtggggctcactttgcaaacacggccaggaagcagacctgcttggactgtaagaaaaatttttgcatgacctgttcgagccaagtagggaatgggccccgcctctgccttctctgccaacggtttcgagctacagcctttcagcgagaggagctcatgaagatgaaggtgaaggacttgagggactatctcagcctccatgacatctctaccgaaatgtgccgggagaaagaagagctggtgctcttggtccttggccagcagcctgtaatctcccaggaggacaggactcgtgcctccaccttgtccccagactttcctgagcagcaggccttcctgacccagcctcactccagcatggttccacctacctcacccaacctcccctcttcatctgcacaagccacctctgttcccccagcccaggttcaggagaatcagcaggccaatggccatgtgtctcaggatcaagaggaacccgtctacctggagagcgtggccagagtacctgctgaggatgagacccagtctattgactcagaggacagctttgtcccaggccgaagggcctctctgtctgacctgactgacctggaggacattgaaggcctgacagtgcggcagctgaaagagatcttggctcgcaactttgtcaactacaagggctgctgtgagaagtgggagctgatggagagagtgacccggctatacaaggatcagaaaggactccagcacctggtcagtggtgccgaagaccaaaacgggggagcagtaccatcaggcttggaggagaacctgtgtaagatctgcatggactcacccattgactgtgttcttctggagtgtggccacatggtaacctgtaccaagtgtggcaagcgcatgaatgaatgtcccatctgccggcagtatgtaatccgagctgtgcatgtcttccggtcctgagagcttgcatcggtttcttcagtgccttacagggaaatagcctggggtgtctgggctcagggttggccagcttgcagaggagcaagctagtagaaatattgcagggttcccaaaaccaggtcaagcaagatgccatgtcacccctgagcatgcctgtcttcccaggggtgtacctcttggctggcaaagcccaaggccagtgggaacttgtataaatcacatgggtatgttcttggttcagtgatcttggagtgatgatggtaactgatgaacagagaactttccagaacttgggtcctgtcttcctccctgaacctagacagtttcacccctcctcctgtacccaacccatccccaccccatatgggagatgcttgcccatgtgtttcatcaatcagaagtcctcctccccagcacattcggatttaaatttctggtctctccggctttctgtgctttaactatctttgctaaagtctcttgctacattgcctaaatccatttgtttctttggaccaaaaatgttagtttaccagaccgatagtggtccttaggacagattttagaccagtaaacttatctttggtgagaaatgaaatacaaatgctatagaaaaatttcaaagttgttaaaagccacttcattaaatgctgggattggaagctgttggtctcctgacctgactctgggagccaacaaaacatcaggctcccaaactgctgggaaatttgtttctgtctgcaagggatagtgcgtgcataatccctgctgctgcttctcttgggacctgatttgcaccatccttagtttatgcggaaaggggaagcaatgggtggaggcagtggccttctccttaattttggcttggcttctgatccttttcccaactagaagtcttaagccacttttcgctgaaaatgaaatccctcccttatccatggtagttttaaatttggtttcattgaagaaatagatttaggcaaagagcctgtgctcttctctggccagactgctgttgaagttcctggagagcaaatgttaagacagagcacttaagagctttcaggaggtactgaatgagactggattctgttgtaagccgacagttcaagtctttatttcccaggttggtggggagtgaggagaccctactttcattccctttcctagcccactgatttttgggggcaggtcagggagaaagcattttttttttttttttgagtctcactctgttgcccaggctggagtgcagtggtacagtcttggcttactgcaacctccacctcccaggttcaagctattctcctgccttagcctccagagtagctgggattataggcgcccaccaccacaccaggctaatttttgtatttttagtagagacggggttttaccatgttcaccaggctggtcttgaactcttgaccttatgatccgcccgcctcagcttcccaaagtgctgggattacaggcgtgagccaccacacccagcaggagaaaccattttctaggctctttaggaaggaccatgcaatgagtaggtgatgtatttgagccctcacagtctttacacccctagaggagctggaggacttaagagtttgctgcagaagcgtggccaagcacaaaacacatgatcaggaaagcagagtgttttcccattaccaatggatgaactatgcaaaccctaaacgccttggcagacacctcccaccccttacccctcccatgcagctgaaaaatctgagactagaaccaatcatgactctggatggcagagggaaacctctaaagggacttattacattggtacagacatatttatgctttcttccatcaccaaccactaaacccctttggaggaatgaaataactgcataaactagtcaactgaacactgggccacttacctcaatgttatacaaagtcctggatgatttgattctgaaccacagcctttgcaggagttgggggaatcagatttgctcatgaagacatccctttccacttttgtcatgggcagtaaatactatagtttacaatgcctaccaattagcaaaggatcattcattcagctactcagttcctctgtaaaacaggtctatgtatgtgcaattcagctaagatctagcagtaacttaagggcagaagctggctctctacttacaacctgctttctctgctgaagccttacctcctcttcagtttccctcctagacacaaatcgaaaataatatactgatagctggttagtaacctcagtaagaattaaaactgaggttgtttactcattttgcctttaaatcttttatccccttttggtgaaggtttccctttaggaaaaaaggtgtcaaacaaccctgattttttttttttttgcatcatttttattacaccaaattaaagttgggagttccaagatcccattccagttaatatttaaccaaggtctaaaatttgattttttttaaatctttgaatcctcccttctgcccctcatggatccttggttttaatgatatggaaacatctaattcttagaattattccgtagcttttgctgattactctgagatttcagttaagacttgtttcaaaagacagatagctgactggttcataatacattggaatagttggatccaaactaataagaataagctgtacaggaactagtgctcaatatacattgtataaatttgtggaaatctcttggatgtgaattgttacttcaagtggcttttattaagattttctcagacttacttggaggttaaagcaaacccaaatgtgtattattttgttacagagctctgctttataattttgtaataaagtttcaatacagacacctgtcctgtctggtgcgaggatgttagaggttgataggactgacaaaagagcctgacagtggttcatgctataatttccacactttaggaggccaaggtgggaagactgcttgagctcaggagtttgagaccaacctgggcaatgtagtgagaccttgtctctacttaaaaaaacaaacaagggccgggtgcggtggctcacgcctgtaatcccagcactttgggaggctgaggcgggcagatcacgaggtcaggagatcgagaccaccctggctaacacggtgaaaccccgtctctactaaaaatacaaaaaattagccaggcgtggtggcgggtgcctgtagtcccagctactcgggaggctgaggcaggagaatggtgtgaacccgggaggcggagcttgcagtgagccaagattgcgccactgcagtccggcctgggcgaaagagcgagactctgtctcaaaaaacaaaacaaaaaaaaacacaaacaaaaaacccaaaaaaaccaaaaaattagccgtgcatggtggtgcatgtgggtagtcccaactacttggaaggctgaggtgggaggatcacttgagccaggagattgaagctgcagtgagctctgatcgccaccgcactccagccagggtgacagagtgagaccctgtattaaaaaaaaaaaaaaattgacaactgataaaccacaagctaatcctgggatgtgcacatggcctgggattagcctgtggttgggccaagtgaccttggtcaagatgctcagtgttgttgtaactaactaacttgctcaagaccacaaatgtaatatatggaagaggccatggaacaacctagaagagaatgagagctgggtaggtaaaggatgaatcccaagaccaattgggttgaagcaccaagtgagttcagatatttacactgatctagaagagatgggccttaaagaactgaaatctgaggacagttagtatgcccagttaggtcttcaggttaccctgggagatacatggtaccttttggatacagctgtagtacagctggactatggctcagaaaaggttggcccaggctgggcgccatggctcacgcctgtaatcccagcactttgggaggctgaggtgcgtggatgactcgaggccaggagtttgagaccagtctggacaacatggcgaaacgtggtctctactaaaaatacaaaaacttagctgcgagtgatggtgtgtgcctgtttgttaagtcttagccacttgggaggctgaggcatgagaattgcttgtgaacccgggaagcagaggttgcagtgagctgagattgcaccactgcactccagcctgaatgacagagcaagagtctgtttaaaaaaaaaaaaaaggttggcccagcctcgtgacagactgctgtgtgtgcgcaggcttgtacaaacctccctgaccaggcgcccaaggagcagggaaaagactactgagaatgcagcccctgggagaacaagattatgtgtccatctgctgtgactgagaagagaaaggaatcttgccaaaagggcataaaggatcgaggatgagccacaacactttgctcacattgccctagggagctgacctgttcagcacccagtgatgacctctgcttgatgccagccagcagcatgtagaggttcagaagagggaggcctccagcattctgctggagagatttatgatgatgatgttgagggctgcattgaaatacatgaaagaaatacatgaaaggatacaaaatagtatatacagtcagatctcagcagcaaagactggaagaaaatacataaaaaaccactgacattgaaatggcaataacacacactaaggcagagaagggacaagatgatcacctgcagccagcagaggccctttcctgtgagtgtgcactctgggtgtaagcaaccaataagggcaagtggggctgggtccaggttggagaggacagtgcctttggcaggatcctgaggagacagccttgttctgctgccctgtccggttgtttgtttggcctgaggctgaagagagaaatggaaatctatgtaggtcagtactcaaaggaggagcagctttttgtctccatagaggatggaataaaaagtatcttattcctaatactgaagcaaggctgaatggtgcctattcaaaaacaccaaatgtcctgggctagagtcaggaaagtaaacccctcccccgtgctgtccctactcccccaacatacacattttaaacagtaggtggaagccaaagtcaatcagatcaaagttgggggggggcggtggggtggccagggggcgcagtggtccttgatcttataggtccagagcctgttaatttgttcataaggtgagaactgaccctcaagagctggaaggattgggacatggcagccagtttacaaccatatggtgtcctgatgagcctgcactcagagtggagtgttggctggaacatgcacacctgcaactgagggttctaaagtcataggttaggcccttctcccgggtagggtctggacatgcacaccaggagtgacgttcccaactctagataactatgtgctaggctctgggctaagtgctttacataatgttgctcaggacaacaggacaaccctatgatttaggtgcacccattctacttggagggattgaggcttagatgttaacgtaacttgcctaaggttagagccattaagtttcaggagtctgatctgaactccgtctacacctgcactgtccagtgtggtagtcactagtgacatgtgaccatttgaagttgaattaaaataagaacttcagttccttagttgtgccagcttcatttcaattgctcagcagctgtctgtggctagtttggttggatactgcagatttatggatatttccactgctgcaaaaagttctattgaatagcacttgtctacactaaattgtctctcttccttttgttcttcaaataaatcagcctcaggtagcacaggagctgttctttgtaagcaggttgtatggatctgttctcaggtgtggatttttatttttttccctgcagatatggagtatattttctttaagaaattaaaaactaagaagaaaatgcca

SEQ ID NO: 50 - protein, 363 aa, Homo sapiens
MWATCCNWFCLDGQPEEVPPPQGARMQAYSNPGYSSFPSPTGLEPSCKSCGAHFANTARKQTCLDCKKNFCMTCSSQVGNGPRLCLLCQRFRATAFQREELMKMKVKDLRDYLSLHDISTEMCREKEELVLLVLGQQPVISQEDRTRASTLSPDFPEQQAFLTQPHSSMVPPTSPNLPSSSAQATSVPPAQVQENQQANGHVSQDQEEPVYLESVARVPAEDETQSIDSEDSFVPGRRASLSDLTDLEDIEGLTVRQLKEILARNFVNYKGCCEKWELMERVTRLYKDQKGLQHLVSGAEDQNGGAVPSGLEENLCKICMDSPIDCVLLECGHMVTCTKCGKRMNECPICRQYVIRAVHVFRS

SEQ ID NO: 51 - DNA, 3229 bp, Homo sapiens
  source 1..3229: mol_type = unassigned DNA
aggcgcgctggggctgcgctgcgccgccggctctgtggcttgccggcttcggggaaggtgcggcaggcggtgctgcggcctggcacagcaggtttggggtgcgggaggcgggcggggtaggagcgcggcgggcggggccgggcggcgggcggggctggcggggcggccgggccatgcagggcgcagagccggctaaaccctgctgagacccggctccgtgcgtccaggggcggctaatgcccctcacgctgtctacgctgctgcaaccgggccgcatctggacggggcgccgcgcggcggagccgacgccgggccacaatgctgcttggagcctctctggtgggggtgctgctgttctccaagctggtgctgaaactgccctggacccaggtgggattctccctgttgttcctctacttgggatctggcggctggcgcttcatccgggtcttcatcaagaccatcaggcgcgatatctttggcggcctggtcctcctgaaggtgaaggcaaaggtgcgacagtgcctgcaggagcggcggacagtgcccattttgtttgcctctaccgttcggcgccaccccgacaagacggccctgatcttcgagggcacagatacccactggaccttccgccagctggatgagtactcaagcagtgtagccaacttcctgcaggcccggggcctggcctcgggcgatgtggctgccatcttcatggagaaccgcaatgagttcgtgggcctatggctgggcatggccaagctcggtgtggaggcagccctcatcaacaccaacctgcggcgggatgctctgctccactgcctcaccacctcgcgcgcacgggcccttgtctttggcagcgaaatggcctcagccatctgtgaggtccatgccagcctggacccctcgctcagcctcttctgctctggctcctgggagcccggtgcggtgcctccaagcacagaacacctggaccctctgctgaaagatgctcccaagcaccttcccagttgccctgacaagggcttcacagataaactgttctacatctacacatccggcaccacagggctgcccaaggccgccatcgtggtgcacagcaggtattaccgcatggctgccctggtgtactatggattccgcatgcggcccaacgacatcgtctatgactgcctccccctctaccactcagcaggaaacatcgtgggaatcggccagtgcctgctgcatggcatgacggtggtgattcggaagaagttctcagcctcccggttctgggacgattgtatcaagtacaactgcacgattgtgcagtacattggtgaactgtgccgctacctcctgaaccagccaccgcgggaggcagaaaaccagcaccaggttcgcatggcactaggcaatggcctccggcagtccatctggaccaacttttccagccgcttccacataccccaggtggctgagttctacggggccacagagtgcaactgtagcctgggcaacttcgacagccaggtgggggcctgtggtttcaatagccgcatcctgtccttcgtgtaccccatccggttggtacgtgtcaacgaggacaccatggagctgatccgggggcccgacggcgtctgcattccctgccagccaggtgagccgggccagctggtgggccgcatcatccagaaagaccccctgcgccgcttcgatggctacctcaaccagggcgccaacaacaagaagattgccaaggatgtcttcaagaagggggaccaggcctaccttactggtgatgtgctggtgatggacgagctgggctacctgtacttccgagaccgcactggggacacgttccgctggaaaggtgagaacgtgtccaccaccgaggtggaaggcacactcagccgcctgctggacatggctgacgtggccgtgtatggtgtcgaggtgccaggaaccgagggccgggccggaatggctgctgtggccagccccactggcaactgtgacctggagcgctttgctcaggtcttggagaaggaactgcccctgtatgcgcgccccatcttcctgcgcctcctgcctgagctgcacaaaacaggaacctacaagttccagaagacagagctacggaaggagggctttgacccggctattgtgaaagacccgctgttctatctagatgcccagaagggccgctacgtcccgctggaccaagaggcctacagccgcatccaggcaggcgaggagaagctgtgattccccccatccctctgagggccggcggatgctggatccggagccccaggttccgccccagagcggtcctggacaaggccagaccaaagcaagcagggcctggcacctccatcctgaggtgctgcccctccatccaaaactgccaagtgactcattgccttcccaacccttccagaggctttctgtgaaagtctcatgtccaagttccgtcttctgggctgggcaggccctctggttcccaggctgagactgacgggttttctcaggatgatgtcttgggtgagggtagggagaggacaaggggtcaccgagcccttcccagagagcagggagcttataaatggaaccagagcagaagtccccagactcaggaagtcaacagagtgggcagggacagtggtagcatccatctggtggccaaagagaatcgtagccccagagctgcccaagttcactgggctccacccccacctccaggaggggaggagaggacctgacatctgtaggtggcccctgatgccccatctacagcaggaggtcaggaccacgcccctggcctctccccactcccccatcctcctccctgggtggctgcctgattatccctcaggcagggcctctcagtccttgtgggtctgtgtcacctccatctcagtcttggcctggctatgaggggaggaggaatgggagagggggctcaggggccaataaactctgccttgagtcctcctagcctgtgtgcaaaccacccaagcccaccctgaccccagaaccccacagccccactgtggccgcttgatcccccacgccaaccccctggcccattgacccgcctcatctgttcattcacttatctaagctgagggtgtagcaggtaagatgccgcagcccctgcctccaatgtgctggttcagccgggggcagtgcccatgtgaatctggcaaggtgtttaacagtgtgggcttgaaagtccaaacca

SEQ ID NO: 52 - protein, 643 aa, Homo sapiens
MLLGASLVGVLLFSKLVLKLPWTQVGFSLLFLYLGSGGWRFIRVFIKTIRRDIFGGLVLLKVKAKVRQCLQERRTVPILFASTVRRHPDKTALIFEGTDTHWTFRQLDEYSSSVANFLQARGLASGDVAAIFMENRNEFVGLWLGMAKLGVEAALINTNLRRDALLHCLTTSRARALVFGSEMASAICEVHASLDPSLSLFCSGSWEPGAVPPSTEHLDPLLKDAPKHLPSCPDKGFTDKLFYIYTSGTTGLPKAAIVVHSRYYRMAALVYYGFRMRPNDIVYDCLPLYHSAGNIVGIGQCLLHGMTVVIRKKFSASRFWDDCIKYNCTIVQYIGELCRYLLNQPPREAENQHQVRMALGNGLRQSIWTNFSSRFHIPQVAEFYGATECNCSLGNFDSQVGACGFNSRILSFVYPIRLVRVNEDTMELIRGPDGVCIPCQPGEPGQLVGRIIQKDPLRRFDGYLNQGANNKKIAKDVFKKGDQAYLTGDVLVMDELGYLYFRDRTGDTFRWKGENVSTTEVEGTLSRLLDMADVAVYGVEVPGTEGRAGMAAVASPTGNCDLERFAQVLEKELPLYARPIFLRLLPELHKTGTYKFQKTELRKEGFDPAIVKDPLFYLDAQKGRYVPLDQEAYSRIQAGEEKL

SEQ ID NO: 53 - DNA, 6783 bp, Homo sapiens
  source 1..6783: mol_type = unassigned DNA
gcagagtgcgggccgggcgggagtgcggcgagagccggctggctgagcttagcgtccgaggaggcggcggcggcggcggcggcacggcggcggcggggctgtggggcggtgcggaagcgagaggcgaggagcgcgcgggccgtggccagagtctggcggcggcctggcggagcggagagcagcgcccgcgcctcgccgtgcggaggagccccgcacacaatagcggcgcgcgcagcccgcgcccttccccccggcgcgccccgccccgcgcgccgagcgccccgctccgcctcacctgccaccagggagtgggcgggcattgttcgccgccgccgccgccgcgcgggccatgggggccgcccggcgcccggggccgggctggcgaggcgccgcgccgccgctgagacgggccccgcgcgcagcccggcggcgcaggtaaggccggccgcgccatggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctggagtccactaaagcgagtgagctggacttggaaaagggcttggagatgagaaaatgggtcctgtcgggaatcctggctagcgaggagacttacctgagccacctggaggcactgctgctgcccatgaagcctttgaaagccgctgccaccacctctcagccggtgctgacgagtcagcagatcgagaccatcttcttcaaagtgcctgagctctacgagatccacaaggagttctatgatgggctcttcccccgcgtgcagcagtggagccaccagcagcgggtgggcgacctcttccagaagctggccagccagctgggtgtgtaccgggccttcgtggacaactacggagttgccatggaaatggctgagaagtgctgtcaggccaatgctcagtttgcagaaatctccgagaacctgagagccagaagcaacaaagatgccaaggatccaacgaccaagaactctctggaaactctgctctacaagcctgtggaccgtgtgacgaggagcacgctggtcctccatgacttgctgaagcacactcctgccagccaccctgaccaccccttgctgcaggacgccctccgcatctcacagaacttcctgtccagcatcaatgaggagatcacaccccgacggcagtccatgacggtgaagaagggagagcaccggcagctgctgaaggacagcttcatggtggagctggtggagggggcccgcaagctgcgccacgtcttcctgttcaccgacctgcttctctgcaccaagctcaagaagcagagcggaggcaaaacgcagcagtatgactgcaaatggtacattccgctcacggatctcagcttccagatggtggatgaactggaggcagtgcccaacatccccctggtgcccgatgaggagctggacgctttgaagatcaagatctcccagatcaagaatgacatccagagagagaagagggcgaacaagggcagcaaggctacggagaggctgaagaagaagctgtcggagcaggagtcactgctgctgcttatgtctcccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaataaggaagatgatgagtctccggggctctatgggtttctgaatgtcatcgtccactcagccactggatttaagcagagttcaaatctgtactgcaccctggaggtggattcctttgggtattttgtgaataaagcaaagacgcgcgtctacagggacacagctgagccaaactggaacgaggaatttgagatagagctggagggctcccagaccctgaggatactgtgctatgaaaagtgttacaacaagacgaagatccccaaggaggacggcgagagcacggacagactcatggggaagggccaggtccagctggacccgcaggccctgcaggacagagactggcagcgcaccgtcatcgccatgaatgggatcgaagtaaagctctcggtcaagttcaacagcagggagttcagcttgaagaggatgccgtcccgaaaacagacaggggtcttcggagtcaagattgctgtggtcaccaagagagagaggtccaaggtgccctacatcgtgcgccagtgcgtggaggagatcgagcgccgaggcatggaggaggtgggcatctaccgcgtgtccggtgtggccacggacatccaggcactgaaggcagccttcgacgtcaataacaaggacgtgtcggtgatgatgagcgagatggacgtgaacgccatcgcaggcacgctgaagctgtacttccgtgagctgcccgagcccctcttcactgacgagttctaccccaacttcgcagagggcatcgctctttcagacccggttgcaaaggagagctgcatgctcaacctgctgctgtccctgccggaggccaacctgctcaccttccttttccttctggaccacctgaaaagggtggcagagaaggaggcagtcaataagatgtccctgcacaacctcgccacggtctttggccccacgctgctccggccctccgagaaggagagcaagctccctgccaaccccagccagcctatcaccatgactgacagctggtccttggaggtcatgtcccaggtccaggtgctgctgtacttcctgcagctggaggccatccctgccccggacagcaagagacagagcatcctgttctccaccgaagtctaaaggtcccagtccatctcctggaggcggacagatggcctggaaacctctggctaatcgggccatccgtagagcgggaaccttcctgaggtgtccttgggccacccccaagtgttgggccatctgccaagagacagcgacccaaagccgaaggacaggtggcctggaaagatcccgcccaggtctgggagccccaggctggcctcagactgtggttttttatgtggccacctgagggcgccccaagccagttcatctcggagtccaggcctggccctgggagacagggtgaagggagtggtttttatgaacttaacttagagtctaaaagatttctactggatcacttgtcaagatgcgccctctctggggagaagggaacgtgactggattccctcactgttgtatcttgaataaacgctgctgcttcatcctgtgggggccgtggccctgtccctgtgtgggtggggcctcttccatttccctgacttagaaaccacactccacttctaacagggtttgagaggcttagtcagcactgggtagcgttttgactccattcttggctttctttttctttccagaaggatttttgtgcagaaatgggtcttttgttgccatgttagtcctccttggaaggcagctcagaaggcctgtgaaatgtcaggggacaggacccccagggagggaaccccaggctacgcactttagggttcgttctccagggagagcgacctcgtcccccgatcctgaccgcccttccggcccacgctctcctgtttggcttccacaggcctggacttctctggcttctctgcccacacactccctgcccccagtgtccctgcccctgccccagcacaggtgacttcatttctgtcctctcagctcagtggactcgctcaacttttgtataagtctccacttggtggcagcagcttgctgatgacttgttttaaaactttcatcctaaataaccttttgatacttgaatatttgtaagttttatacatagtttctaatttttttccgaacagatccagatacctaataagatgctggaatgtaatccctggacaatccgtgtcctggcagcatttggtcttcctctaagcgcctggctccgctgttctcaggagtgggttctgaagtctctggagaacaggatacgtggagggttaggaaggggccaggcctagagacgggagactccctcccggagcaggtggaggcacaggaccattcgctaccccatctgccgacacctgcgggggagcccaggcattctttgtaaaccctcctgaccacctggctcaaagaaaacagaagcatggagaccgccaagtattttcaagaaataaccccatgaatattccatcacttttttagaaagaggggcttggggcaggcagaggagagaagggagagcaaactgagagccaagtttccacacggtcctgcaggaggagaggatgcagctgcccagagggaagcaggatcacatttaaggaagtgtgtggggtccctggatgacaccagcacccagtgcggctctgtctggcacccgctcccaaggtgggaggagtgggtgtcccctgtgtgtcagtgggcagctcctgctgaacccgcagctcactagggagcctgacagtggggccatgcgcctgacactcctctctgcttgtggacctggcaaggcagggagcagaaaacagccacttgaaggctttctgtctgcgtctgtgtgcagtgtggatttagttgtgcttttttcttgctgggagagcacagccaccatttacaagcagtgtcaccgtcgtgggtggcgaggacagaacaggagcctctgctctctgtacctatctgggcccggtgggctcccttgtcctggcttccatctctgtctcagcgaccattcagccctgcgcaggaacacgtgttgcttagaaaagccaaatccagccttgtctctgcctcctctggtctcatgatgtgcatctgttaccttgaaactggaaaccagtctatcaatgtctgtgccaattttttattccctccccaacctccttccccatacgactttttatttatgtaggatgtgtgctgtctaatgatgggatgaccacacttttccatgttctaaaagtgctcctctcccgcagggtcccagggctggtggttgctttgggtctacagctacgtcttacccacctcctgcctcaacagcctgtgtggtggcaaagccggtgtggggctggggaacgcagcgttctccaggagggggacctggctctccttctgcaatgcaggcgaaggcctagatgccagtgtgacctcccacaaggcgtggcttccagactccccggccggaagtgatgcttttttgccttgggccctgggtttgaagcagcctggctttctcttggtaagtggctggtgtcttagcagctgcaatctgagctcagccacctacacaccaccgtggccgacactttcattaaaaagtttcctgagacga

SEQ ID NO: 54 - protein, 1271 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGMMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDGEGAFHGDADGSFGTPPGYGCAADRAEEQRRHQDGLPYIDDSPSSSPHLSSKGRGSRDALVSGALESTKASELDLEKGLEMRKWVLSGILASEETYLSHLEALLLPMKPLKAAATTSQPVLTSQQIETIFFKVPELYEIHKEFYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTDLLLCTKLKKQSGGKTQQYDCKWYIPLTDLSFQMVDELEAVPNIPLVPDEELDALKIKISQIKNDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEDDESPGLYGFLNVIVHSATGFKQSSNLYCTLEVDSFGYFVNKAKTRVYRDTAEPNWNEEFEIELEGSQTLRILCYEKCYNKTKIPKEDGESTDRLMGKGQVQLDPQALQDRDWQRTVIAMNGIEVKLSVKFNSREFSLKRMPSRKQTGVFGVKIAVVTKRERSKVPYIVRQCVEEIERRGMEEVGIYRVSGVATDIQALKAAFDVNNKDVSVMMSEMDVNAIAGTLKLYFRELPEPLFTDEFYPNFAEGIALSDPVAKESCMLNLLLSLPEANLLTFLFLLDHLKRVAEKEAVNKMSLHNLATVFGPTLLRPSEKESKLPANPSQPITMTDSWSLEVMSQVQVLLYFLQLEAIPAPDSKRQSILFSTEV

SEQ ID NO: 55 - DNA, 7568 bp, Homo sapiens
  source 1..7568: mol_type = unassigned DNA
gaggtcaactgcgcgccgctggcgctgaggggaggaagtttgctgtcgagcggcctgggttccgtgggcaaggccgtgggaggcagcgttggctgcttcgacacactgagggcggcgcgatgggagacgagatggatgccatgattcccgagcgggagatgaaggattttcagtttagagcgctaaagaaggtgagaatctttgactcccctgaggaattgcccaaggaacgctcgagtctgcttgctgtgtccaacaaatatggtctggtcttcgctggtggagccagtggcttgcagatttttcctactaaaaatcttcttattcaaaataaacccggagatgatcccaacaaaatagttgataaagtccaaggcttgctagttcctatgaaattcccaatccatcacctggccttgagctgtgataacctcacactctctgcgtgcatgatgtccagtgaatatggttccattattgctttttttgatgttcgcacattctcaaatgaggctaaacagcaaaaacgcccatttgcctatcataagcttttgaaagatgcaggaggcatggtgattgatatgaagtggaaccccactgtcccctccatggtggcagtttgtctggctgatggtagtattgctgtcctgcaagtcacggaaacagtgaaagtatgtgcaactcttccttccacggtagcagtaacctctgtgtgctggagccccaaaggaaagcagctggcagtgggaaaacagaatggaactgtggtccagtatcttcctactttgcaggaaaaaaaagtcattccttgtcctccgttttatgagtcagatcatcctgtcagagttctggatgtgctgtggattggtacctacgtcttcgccatagtgtatgctgctgcagatgggaccctggaaacgtctccagatgtggtgatggctctactaccgaaaaaagaagaaaagcacccagagatatttgtgaactttatggagccctgttatggcagctgcacggagagacagcatcattactacctcagttacattgaggaatgggatttagtgctggcagcatctgcggcttcaacagaagttagtatccttgctcgacaaagtgatcagattaattgggaatcttggctactggaggattctagtcgagctgaattgcctgtgacagacaagagtgatgactccttgcccatgggagttgtcgtagactatacaaaccaagtggaaatcaccatcagtgatgaaaagactcttcctcctgctccagttctcatgttactttcaacagatggtgtgctttgtccattttatatgattaatcaaaatcctggggttaagtctctcatcaaaacaccagagcgactttcattagaaggagagcgacagcccaagtcaccaggaagtactcccactaccccaacctcctctcaagccccacagaaactggatgcttctgcagctgcagcccctgcctctctgccaccttcatcacctgctgctcccattgccactttttctttgcttcctgctggtggagcccccactgtgttctcctttggttcttcatctttgaagtcatctgctacggtcactggggagcccccttcatattccagtggctccgacagctccaaagcagccccaggccctggcccatcaaccttctcttttgttcccccttctaaagcctccctagcccccacccctgcagcgtctcctgtggctccatcagctgcttcattctcctttggatcatctggttttaagcctaccctggaaagcacaccagtgccaagtgtgtctgctccaaatatagcaatgaagccctccttcccaccctcaacctctgctgtcaaagtcaaccttagtgaaaagtttactgctgcagctacctctactcctgttagtagctcccagagcgcacccccgatgtcgccattctcttctgcctccaagccagctgcttctggaccactcagccaccccacacctctctcagcaccacctagttccgtgccattgaagtcctcagtcttgccctcaccatcaggacgatctgctcagggcagttcaagcccagtgccctcaatggtacagaaatcacccaggataacccctccagcggcaaagccaggctctccccaggcaaagtcacttcagcctgctgttgcagaaaagcagggacatcagtggaaagattcagatcctgtaatggctggaattggggaggagattgcacactttcagaaggagttggaagagttaaaagcccgaacttccaaagcctgtttccaagtgggcacttctgaggagatgaagatgctgcgaacagaatcagatgacttgcatacctttcttttggagattaaagagaccacagagtcgcttcatggagatataagtagcctgaaaacaactttacttgagggctttgctggtgttgaggaagccagagaacaaaatgaaagaaatcgtgactctggttatctgcatttgctttataaaagaccactggatcccaagagtgaagctcagcttcaggaaattcggcgccttcatcagtatgtgaaatttgctgtccaagatgtgaatgatgttctagacttggagtgggatcagcatctggaacaaaagaaaaaacaaaggcacctgcttgtgccagagcgagagacactgtttaacaccctagccaacaatcgggaaatcatcaaccaacagaggaagaggctgaatcacctggtggatagtcttcagcagctccgcctttacaaacagacttccctgtggagcctgtcctcggctgttccttcccagagcagcattcacagttttgacagtgacctggaaagcctgtgcaatgctttgttgaaaaccaccatagaatctcacaccaaatccttgcccaaagtaccagccaaactgtcccccatgaaacaggcacaactgagaaacttcttggccaagaggaagaccccaccagtgagatccactgctccagccagcctgtctcgatcagcctttctgtctcagagatattatgaagacttggatgaagtcagctcaacgtcatctgtctcccagtctctggagagtgaagatgcacggacgtcctgtaaagatgacgaggcagtggttcaggcccctcggcacgcccccgtggttcgcactccttccatccagcccagtctcttgccccatgcagcaccttttgctaaatctcacctggttcatggttcttcacctggtgtgatgggaacttcagtggctacatctgctagcaaaattattcctcaaggggccgatagcacaatgcttgccacgaaaaccgtgaaacatggtgcacctagtccttcccaccccatctcagccccgcaggcagctgccgcagcagcactcaggcggcagatggccagtcaggcaccagctgtaaacactttgactgaatcaacgttgaagaatgtccctcaagtggtaaatgtgcaggaattgaagaataaccctgcaaccccttctacagccatgggttcttcagtgccctactccacagccaaaacacctcacccagtgttgaccccagtggctgctaaccaagccaagcaggggtctctaataaattcccttaagccatctgggcctacaccagcatccggtcagttatcatctggtgacaaagcttcagggacagccaagatagaaacagctgtgacttcaaccccatctgcttctgggcagttcagcaagcctttctcattttctccatcagggactggctttaattttgggataatcacaccaacaccgtcttctaatttcactgctgcacaaggggcaacaccctccactaaagagtcaagccagccggacgcattctcatctggtgggggaagcaaaccttcttatgaggccattcctgaaagctcacctccctcaggaatcacatccgcatcaaacaccaccccaggagaacctgccgcatctagcagcagacctgtggcaccttctggaactgctctttccaccacctctagtaagctggaaaccccaccgtccaagctgggagagcttctgtttccaagttctttggctggagagactctgggaagtttttcaggactgcgggttggccaagcagatgattctacaaaaccaaccaataaggcttcatccacaagcctaactagtacccagccaaccaagacgtcaggcgtgccctcagggtttaattttactgcccccccggtgttagggaagcacacggagccccctgtgacatcctctgcaaccaccacctcagtagcaccaccagcagccaccagcacttcctcaactgccgtttttggcagtctgccagtcaccagtgcaggatcctctggggtcatcagttttggtgggacatctctaagtgctggcaagactagtttttcatttggaagccaacagaccaatagcacagtgcccccatctgccccaccaccaactacagctgccactccccttccaacatcattccccacattgtcatttggtagcctcctgagttcagcaactaccccctccctgcctatgtccgctggcagaagcacagaagaggccacttcatcagctttgcctgagaagccaggtgacagtgaggtctcagcatcagcagcctcacttctagaggagcaacagtcagcccagcttccccaggctcctccgcaaacttctgactctgttaaaaaagaacctgttcttgcccagcctgcagtcagcaactctggcactgcagcatctagtactagtcttgtagcactttctgcagaggctaccccagccaccacgggggtccctgatgccaggacggaggcagtaccacctgcttcctccttttctgtgcctgggcagactgctgtcacagcagctgctatctcaagtgcaggccctgtggccgtcgaaacatcaagtacccccatagcctccagcaccacgtccattgttgctcccggcccatctgcagaggcagcagcatttggtaccgtcacttctggctcatccgtctttgctcagcctcctgctgccagttctagctcagctttcaaccagctcaccaacaacacagccactgccccctctgccacgcccgtgtttgggcaagtggcagccagcaccgcaccaagtctgtttgggcagcagactggtagcacagccagcacagcagctgccacaccacaggtcagcagctcagggtttagcagcccagcttttggtaccacagccccaggggtctttggacagacaaccttcgggcaggcctcagtctttgggcagtcggcgagcagtgctgcaagtgtcttttccttcagtcagcctgggttcagttccgtgcctgccttcggtcagcctgcttcctccactcccacatccaccagtggaagtgtctttggtgccgcctcaagtaccagtagctccagttccttctcatttggacagtcttctcccaacacaggaggggggctgtttggccaaagcaacgctcctgcttttgggcagagtcctggctttggacagggaggctctgtctttggtggtacctcagctgccaccacaacagcagcaacctctgggttcagcttttgccaagcttcaggttttgggtctagtaatactggttctgtgtttggtcaagcagccagtactggtggaatagtctttggccagcaatcatcctcttccagtggtagcgtgtttgggtctggaaacactggaagagggggaggtttcttcagtggccttggaggaaaacccagtcaggatgcagccaacaaaaacccattcagctcggccagtgggggctttggatccacagctacctcaaatacctctaacctatttggaaacagtggggccaagacatttggtggatttgccagctcgtcgtttggagagcagaaacccactggcactttcagctctggaggaggaagtgtggcatcccaaggctttgggttttcctctccaaacaaaacaggtggcttcggtgctgctccagtgtttggcagccctcctacttttgggggatcccctgggtttggaggggtgccagcattcggttcagccccagcctttacaagccctctgggctcgacgggaggcaaagtgttcggagagggcactgcagctgccagcgcaggaggattcgggtttgggagcagcagcaacaccacatccttcggcacgctcgcgagtcagaatgcccccactttcggatcactgtcccaacagacttctggttttgggacccagagtagcggattctctggttttggatcaggcacaggagggttcagctttgggtcaaataactcgtctgtccagggttttggtggctggcgaagctgagggcgtgtcagcaggcctttcgatccctgggaccaaccgcatcctcagcttcttccccgagaaatgctggagcaggctgttcagaccgacgttgccatcaaaacacatacacccagaaagaaacaacagaaaccaaaactcacaaggcgcatgattacttgttttatatttcatgttgggttttccctcccactattaaacagtctgtttccgtacagaacgtatgtgggttttttcagatcacagccaagaagattgccccgtttctgtggctctgagaagccagcagagcctccatcagccagaacactgtgtcttcaaggatggcatcagaagtcaccagcgtcgggtgttgataaacagcatcgaatgtgccgtggtctcacttggacctagcgccctccacatagggaagaggttgaatgctggtgggtcatctttttgaggctgaaacttggttatctcctctccttgttcttttagccattgtgtatcaggaccatccaaggacgcactctgcgattgagtggaggcagaggaagccactcatgccagcagcagttgagtttcagaagcagccatagcgcttttcagtacagtacaatagtagccagcgtgaggcaggacagtgctgcgccttagccggccttggctctcatctccatgtggctcttgggtgccatgtgtcctactccagagaggagggggccggtcacttgagaagacagcctttatattctgtatgtggcagtgacattggtagccacacctacccagtgtctactaaccacagtgcagatagcccttgggtagacccaggaagtgtgaagacagacactgctgttgccagacaacactagcagaacgatgctggatttgacaacagtgtttgctaggacggcccagagctcatcactgcagttttattagagtgcttctttatctttaatgcagtatctttgaggcctgtaacatcctagatagttgccatcaaacaaggaccgtctcgcattgaaaacagatgtaagaatttctctttcaggcccagaatctgacagtgctttggcttgggtcatgagcagcggggagttgggaagagatttttttttttagagttttacatattggcaggttgtatttttttaatgttttaataaaagtttgacatgacattaa

SEQ ID NO: 56 - protein, 2090 aa, Homo sapiens
MGDEMDAMIPEREMKDFQFRALKKVRIFDSPEELPKERSSLLAVSNKYGLVFAGGASGLQIFPTKNLLIQNKPGDDPNKIVDKVQGLLVPMKFPIHHLALSCDNLTLSACMMSSEYGSIIAFFDVRTFSNEAKQQKRPFAYHKLLKDAGGMVIDMKWNPTVPSMVAVCLADGSIAVLQVTETVKVCATLPSTVAVTSVCWSPKGKQLAVGKQNGTVVQYLPTLQEKKVIPCPPFYESDHPVRVLDVLWIGTYVFAIVYAAADGTLETSPDVVMALLPKKEEKHPEIFVNFMEPCYGSCTERQHHYYLSYIEEWDLVLAASAASTEVSILARQSDQINWESWLLEDSSRAELPVTDKSDDSLPMGVVVDYTNQVEITISDEKTLPPAPVLMLLSTDGVLCPFYMINQNPGVKSLIKTPERLSLEGERQPKSPGSTPTTPTSSQAPQKLDASAAAAPASLPPSSPAAPIATFSLLPAGGAPTVFSFGSSSLKSSATVTGEPPSYSSGSDSSKAAPGPGPSTFSFVPPSKASLAPTPAASPVAPSAASFSFGSSGFKPTLESTPVPSVSAPNIAMKPSFPPSTSAVKVNLSEKFTAAATSTPVSSSQSAPPMSPFSSASKPAASGPLSHPTPLSAPPSSVPLKSSVLPSPSGRSAQGSSSPVPSMVQKSPRITPPAAKPGSPQAKSLQPAVAEKQGHQWKDSDPVMAGIGEEIAHFQKELEELKARTSKACFQVGTSEEMKMLRTESDDLHTFLLEIKETTESLHGDISSLKTTLLEGFAGVEEAREQNERNRDSGYLHLLYKRPLDPKSEAQLQEIRRLHQYVKFAVQDVNDVLDLEWDQHLEQKKKQRHLLVPERETLFNTLANNREIINQQRKRLNHLVDSLQQLRLYKQTSLWSLSSAVPSQSSIHSFDSDLESLCNALLKTTIESHTKSLPKVPAKLSPMKQAQLRNFLAKRKTPPVRSTAPASLSRSAFLSQRYYEDLDEVSSTSSVSQSLESEDARTSCKDDEAVVQAPRHAPVVRTPSIQPSLLPHAAPFAKSHLVHGSSPGVMGTSVATSASKIIPQGADSTMLATKTVKHGAPSPSHPISAPQAAAAAALRRQMASQAPAVNTLTESTLKNVPQVVNVQELKNNPATPSTAMGSSVPYSTAKTPHPVLTPVAANQAKQGSLINSLKPSGPTPASGQLSSGDKASGTAKIETAVTSTPSASGQFSKPFSFSPSGTGFNFGIITPTPSSNFTAAQGATPSTKESSQPDAFSSGGGSKPSYEAIPESSPPSGITSASNTTPGEPAASSSRPVAPSGTALSTTSSKLETPPSKLGELLFPSSLAGETLGSFSGLRVGQADDSTKPTNKASSTSLTSTQPTKTSGVPSGFNFTAPPVLGKHTEPPVTSSATTTSVAPPAATSTSSTAVFGSLPVTSAGSSGVISFGGTSLSAGKTSFSFGSQQTNSTVPPSAPPPTTAATPLPTSFPTLSFGSLLSSATTPSLPMSAGRSTEEATSSALPEKPGDSEVSASAASLLEEQQSAQLPQAPPQTSDSVKKEPVLAQPAVSNSGTAASSTSLVALSAEATPATTGVPDARTEAVPPASSFSVPGQTAVTAAAISSAGPVAVETSSTPIASSTTSIVAPGPSAEAAAFGTVTSGSSVFAQPPAASSSSAFNQLTNNTATAPSATPVFGQVAASTAPSLFGQQTGSTASTAAATPQVSSSGFSSPAFGTTAPGVFGQTTFGQASVFGQSASSAASVFSFSQPGFSSVPAFGQPASSTPTSTSGSVFGAASSTSSSSSFSFGQSSPNTGGGLFGQSNAPAFGQSPGFGQGGSVFGGTSAATTTAATSGFSFCQASGFGSSNTGSVFGQAASTGGIVFGQQSSSSSGSVFGSGNTGRGGGFFSGLGGKPSQDAANKNPFSSASGGFGSTATSNTSNLFGNSGAKTFGGFASSSFGEQKPTGTFSSGGGSVASQGFGFSSPNKTGGFGAAPVFGSPPTFGGSPGFGGVPAFGSAPAFTSPLGSTGGKVFGEGTAAASAGGFGFGSSSNTTSFGTLASQNAPTFGSLSQQTSGFGTQSSGFSGFGSGTGGFSFGSNNSSVQGFGGWRS

SEQ ID NO: 57 - DNA, 3374 bp, Homo sapiens
  source 1..3374: mol_type = unassigned DNA
acacgcagctagccggagcccggaccaggcgcctgtgcctcctcctcgtccctcgccgcgtccgcgaagcctggagccggcgggagccccgcgctcgccatgtcgggcgagctcagcaacaggttccaaggagggaaggcgttcggcttgctcaaagcccggcaggagaggaggctggccgagatcaaccgggagtttctgtgtgaccagaagtacagtgatgaagagaaccttccagaaaagctcacagccttcaaagagaagtacatggagtttgacctgaacaatgaaggcgagattgacctgatgtctttaaagaggatgatggagaagcttggtgtccccaagacccacctggagatgaagaagatgatctcagaggtgacaggaggggtcagtgacactatatcctaccgagactttgtgaacatgatgctggggaaacggtcggctgtcctcaagttagtcatgatgtttgaaggaaaagccaacgagagcagccccaagccagttggcccccctccagagagagacattgctagcctgccctgaggaccccgcctggactccccagccttcccaccccatacctccctcccgatcttgctgcccttcttgacacactgtgatctctctctctctcatttgtttggtcattgagggtttgtttgtgttttcatcaatgtctttgtaaagcacaaattatctgccttaaaggggctctgggtcggggaatcctgagccttgggtcccctccctctcttcttccctccttccccgctccctgtgcagaagggctgacatcaaaccaaaaactagagggggcagggccagggcagggaggcttccagcctgtgttcccctcacttggaggaaccagcactctccatcctttcagaaagtctccaagccaagttcaggctcactgacctggctctgacgaggaccccaggccactctgagaagaccttggagtagggacaaggctgcagggcctctttcgggtttccttggacagtgccatggctccagtgctctggtgtcacccaggacacagccactcggggccccgctgccccagctgatccccactcattccacacctcttctcatcctcagtgatgtgaaggtgggaaggaaaggagcttggcattgggagcccttcaagaaggtaccagaaggaaccctccagtcctgctctctggccacacctgtgcaggcagctgagaggcagcgtgcagccctactgtcccttactggggcagcagagggcttcggaggcagaagtgaggcctggggttttgggggaaaggtcagctcagtgctgttccaccttttagggaggttactgaggggaccaggatgggagaatgaggagtaaaatgctcacggcaaagtcagcagcactggtaagccaagactgagaaatacaaggttgcttgtctgaccccaatctgcttgaaacctgactctgcttctctcatttgtcttcctaccctactcacataattcactcattgactcactcattcaccagatatttattgacctgctattataagctttacatcctcccatgttgtcctggcatgtgcagtatacacggtctaactcatctctccccagatctctcagaaccttgagcttgggaattgaactggggtcacctgtgtcctttcttatggactcgcaggattttagaaccctaatgcaccctggagggtagctgggccagacttctcatttcacaggtgaggagactggtgccccacagggattaagtgccttgcccaaggtcaggcttatctccagagggaggtgccctggactggggcccagatgttcagggaccctgcctacacctcatttccagtgtgggctgccttagttagttatgagaacagggaagggctgggaagagacagcctccaaggtcaacacttggagagggtttcacttgctctgaagaccctggtccaggattcgccctctcccatgccttcaagtcagcatcaggcttagggcaaagaccaggcctctgaagctgcctcttgtaattcatgcaggaagatgtcaaagtcagccccatcttggctgatcagggtgttcagccttaaccccacctgtgttctgaagtctcttaccctacctgctcaggactgagacagttattcactgaacatatttattaagcacttgctgtaggccaacagttaagaatccaataatgaaatggacagattcatggaacttagagtccaataggaaagtgagacccagacaatgacaatgagataaatgttaggaagggggaggtatggggtgacttccctgcagtcctgggggcctagatgggcccaagactgggtgagagtcttggcagagcctttgcaacaccttaagtggacaggactgggaggtcttggtggttggagccaacgtgggttccctgcggctccttagtcacctctgatagcagattgagggaggaaaacaggtaaggcatgaggaaatggccaggttgggttaacccactggtttcaaccagttcaggaatgaggttatttggccatgactggctgatcttgagctcaaggatctgcttcaaatgcacacaggcctagttgaagtttaaaccccagcaaaacattcctccctgtaaatggaaaatcctacttctacccccaccctgccctgttttttgttttttttttccccaagatcattagatgtcctcacccctcctcactgcctctcctctctgggacaggctgggacctttgaggaagataaagccttccttcactacccatcatattcagtgtccctgttcctcactcagagaggaaggcagaaccagtcaggcttatttcagtaagttccacagttctacaagactgcaggaattctccttaagggaggagagcaagcaggtgtggccccagcttctggaaatggcagaagagagggttttctcattgaatgggggtgggggctcgtgtgtcctgggaaaccccatcagtcccttcatttcttgagactcaactcctgggaggagagggtctcaagagttgtccctggaaggagggcgggggcagtctgcatctatttcaggttgtggctcttggttctaggactcttacttctctggctaagggctcagcttcttgggacttcaaccatcttctttctgaaagaccaaatctaatgtaaccagtaacgtgaggactgccaagtatggctttgtccctatgactcagaggagggtttgtcgggcaaattcaggtggatgaagtatgtgtgtgcgtgtgcatgggagtgtgcgtggactgggatatcatctctacagcctgcaaataaaccagacaaactta

SEQ ID NO: 58 - protein, 150 aa, Homo sapiens
MSGELSNRFQGGKAFGLLKARQERRLAEINREFLCDQKYSDEENLPEKLTAFKEKYMEFDLNNEGEIDLMSLKRMMEKLGVPKTHLEMKKMISEVTGGVSDTISYRDFVNMMLGKRSAVLKLVMMFEGKANESSPKPVGPPPERDIASLP

SEQ ID NO: 59 - DNA, 5443 bp, Homo sapiens
  source 1..5443: mol_type = unassigned DNA
ctctcgcgcagggcccccgcggccggggcagtcccgcagccgagcgcagccgggcgcgcgccaccgcccactcgccctgtgcccgccgcagcccgaaactggccacggccgggagcggaggggacagcggggatcgtgagctccggcccgggcgagcgggtgcgtctgccgcagagtcggcacctgaaggacatggaggaaagaccggcagagaccaatgccaatgtggacaactcggcgtccccctcggtggcccagctggccgggcggtttagggagcaggcggctgcagccaaggagacaccagccagtaaaccaacccgaaggaaaccgccctgttccctccccctgttcccccccaaggtagacctgggccagaatggtgaggagaaatcaccacccaatgcgagccaccctcctaaattcaaggtcaagagctcgcctctgattgagaagcttcaggccaatttaacctttgacccagctgctctactgcctggggcctcacccaagagtcctggactcaaggctatggtgtcgccatttcacagcccaccttctacccccagcagccctggtgtgcgatctaggcccagcgaggcagaggaggtgcctgtcagcttcgaccagccccctgaaggcagtcatctgccctgttacaacaaggtgcggacgaggggctcaataaaaaggcgccctccctccaggcgattccgaaggtcacagtcagactgtggagaacttggagatttcagggcggtggagtcatctcagcagaacggtgctaaggaagaggatggggatgaagtgttgccatccaagagcaaggccccaggatcccctttgtccagtgagggagcagcgggagagggagtgagaaccctgggacctgctgaaaagcctcctctgaggaggtcacccagcaggacagagaagcaggaggaggacagggccacagaggaagccaagaacggtgaaaaggccaggcggagttcagaggaggtggacggccagcacccggcccaagaggaggtcccggaatcgccccagacctctggcccagaggcagaaaataggtgtgggagccccagggaggaaaagccagctggagaggaagcagagatggaaaaggctacagaggtgaagggggagagggtgcaaaatgaagaggtgggacctgaacatgacagccaagaaacaaagaagctggaggagggagctgcagtgaaggagaccccccacagtccccctggaggagtgaagggcggagatgtccccaagcaggaaaaaggcaaggaaaaacaacaggagggggcagtgctcgagccaggctgcagcccccagaccggccctgcccagctggagaccagcagtgaggtccagagcgagccagcagtccccaagccggaggatgacactcctgtccaggacactaaaatgtgaagaacagctcattgtgccccagtgatgaagttgctggacacatctctttgcaggtagcagcaacagttgtagcagcagcagacgaagccattgcagaggcagaatatgctgagtgtctggagtcagcctgaagacacagggtggattatttcctggcctccacaccaaacgttcccttgcagatggagactgaatctgagggcagcagacttttatcagcttgagtttatgtcatttgatggacttggttcaacaacaagaacttacttaaaacaatgtactgtggtgatgagtcccaggggcactggtcagcctgtggagccctggatgctatccacacccacctatccctgcagctaatttagctgatctctaatttaactgagctctaatttagctgatcagattttgcttgggtaaagttcctttttaatgttctaaagtgtttacggttctcaaatatcagttaaaaactaattttaggtggccataaacataaaatagaaaccctgtaagttacagaagaccctaaattgtatcaaaaccctagagacaacttttcaatttgatccaaatttgaactggccaaccagtctttaaaacactggactagaagagataatgattgaaacatttaaaaaaaaaaagtgctccattcgcaggagcttttcctgtcctgtggttttccagttggtgaccaccatgggaggtcgctggctcggctcactcccttctcccacccttgagaatgtggagaactcccatggagaggcagaatggcaggaggtttcatgtcccgcgttgcatctcctcctgaaagaaaagcagtgatacctgaataatgctggctctccgattgatcctgtgaggatgaatttgcatttccagaatccttgagcatggattagatgtttcctgggaggtgccttgagtaccattatgtgcaagctacataattaaaacatttttcttagtttccctgggaagcttttcttgactcacagcccaggttcttctgcccaacacaaaaggagtgagttggggtctttagtctcttcttattgggtagctcttgctttaatattctgtttggtgagtgtaagggattctgcaagggacagggggcctgactacccagtctttgacttgtatcctctcccctcttcatacactcctgctgaaaaatgttaatccaaatacacatttaaacttagggtcggtccttattctgatttgagtattttaatgtctcagtgtgctgatttggtagttggaagaattattcttctggaggtctgttagactacatcctacactgacttcagaaaacagtctgtcagacaaaaaggccttatgtcaccactggtacctcagtttcctcatcccatttacagtttttctaactccagggtagtgtttagtgttaatatttgggatatattttttttcaaaactgtttttaagtagtttgtaatttgtaacaaacttgtaacctggttgggactgatattgtcatagctatgataaactttggatattagcagaatttgggagatgtgtgtgtcaattattttgagatctataaacttgtctgtctatctatctagctggctagcttgctatcaatctagctaccatccatttattgacagagaagtctacccctaaacaatacttggttttatttatatatgtctttgggttgtttatacacatgcactatctgggcaagtactttgtgaatatgtgcatgattgtgaatgtgtgatgaaaagggccactttacttgaggaaggcatggaacagaaagtatggggtttagatatgaaaaatgaaactgcatttttcttctggagggtctaaacatagtagcagacgagagaggggcagtaaaaatgacaagaatgtgacattgcggcaccatgcttgtgggcatcagaatacctttctatcccccaagtctcctgttagtcacattatttggtgtattttgtgaaaaaaatagaggccccattcccagggcttgttcattaataaagggggctcttggcagggaactcatgaagtacagggactgatttttgttggtgctgcagccccactgcttaaaactctcctttctcaaagcaaccatgatcaggtgttttatttgcatggttgatactggaacttctaaatttgggattaaagaaattcatctgtgtcttaggtagcacaaagttattaacaggccaggaagaccttgtttctttcaggcttccataatgggtttgaaagaaaacacggattcttctacagcaacagagttaaagaaatagtcatagtcattcttccacgatctttgtagtctagcttgaatttcaaactgtgtccttccttactccagagagggagcaagaaaaaaaacctggatgctttgaagggatctgccactatttaaaaggggattaggattctgtttcagctgcatgtggaactaggtaagcttcagtggcctcagaactccaggcccctcacggagatcaccctgaacaatgcccagtttttcatgcaggttttgtcctatgaccagttaactacatgttcaataaataaatgtcaaagaaataaatgcgtgaaaaggcaaccagttcatcatcttatgattaggttttcttgattagggatgtgtttagtacagctgaatgaacaagttcacccagctgctggtgacagaaacccaactcaaactagctgatgaaaaacagaactatacagatcaatgaatctctccccctttttcagcctcatagaactgaaaaaaacaaacctaaatacagtcacccaagtgatgtcaggaatctctcatggtattggtctctgctttccctcgtattggccatattcttttttttttcttttttaaaaacttatttaattagttttccttaatagttgcagccaaggctgtctcttttttctcttcttttctcttctttttttttgaatgagacagagtctcgcgctgtcactcaggctggagtgcagtggcacaatcacagttcactgcaacctctgcctcctgcattcaagcgattctcgtgcctcagcctcccaagtactgcagttacttttgcaccaacctaatagctcggattacaagtatgcaccaccatgcccagctaatttttgtattttttagtagagaccaggtttcactatgttggccaggctggtctcaaactcctggcctctagtgatctgcctgcctcggtctcccaaagtgggattacaggcatgaaccaccacgtccagccagtcattttcttaagtaggttcttcccagcctgcaaacccagtgaaagggcttattctttcaccattactgtggggaaaaagtcctgtagaggctttccataggccctgtgtgactcacgtgtctatacctgaatcaactgttacaaccaagcagatgcagagactgcgtgccctctcctggagctaggagctaggtgagtcaatcctgcactttctctacaaggcttgcaaaggtgagcaagaagattccccaaagtgaaatcagggtgctgttaccaaaagaagctatgctggcaggcaaaaaacaacagctgtttcccatagggcaaaacctgaaacacccaattactatcccattgtttgcttcaaaattaaattttgtttgaccccaactagttctagtcaagtttttgctcgatggaccacgtgaaaaaaggataaacaatgtctctatataatgcatgatcaggtggtttatgaagatgtcaagagcactgaacaaaatccaaatgctcacataaaatgggattaggtttcataagccacccaagaaaatcagtctcattattttatgtaataatgtattactgtgcccagagctaacctgtagctctaggggagtacaatgtgtttgtattattcttcattgtacacagtgcctcagacatagtaggtattcaataaatatttgttaaatcatgaa

SEQ ID NO: 60 - protein, 416 aa, Homo sapiens
MEERPAETNANVDNSASPSVAQLAGRFREQAAAAKETPASKPTRRKPPCSLPLFPPKVDLGQNGEEKSPPNASHPPKFKVKSSPLIEKLQANLTFDPAALLPGASPKSPGLKAMVSPFHSPPSTPSSPGVRSRPSEAEEVPVSFDQPPEGSHLPCYNKVRTRGSIKRRPPSRRFRRSQSDCGELGDFRAVESSQQNGAKEEDGDEVLPSKSKAPGSPLSSEGAAGEGVRTLGPAEKPPLRRSPSRTEKQEEDRATEEAKNGEKARRSSEEVDGQHPAQEEVPESPQTSGPEAENRCGSPREEKPAGEEAEMEKATEVKGERVQNEEVGPEHDSQETKKLEEGAAVKETPHSPPGGVKGGDVPKQEKGKEKQQEGAVLEPGCSPQTGPAQLETSSEVQSEPAVPKPEDDTPVQDTKM

SEQ ID NO: 61 - DNA, 6480 bp, Homo sapiens
  source 1..6480: mol_type = unassigned DNA
ctccgtctgaaaaccttggccgaaagggttgtagacatggcggccatcaggatgggaaaactgacaaccatgcctgcaggtctgatatatgcatctgtaagtgtacatgcagccaaacaagaggaatccaaaaagcagctagtgaaaccagagcagctccccatatatactgcaccaccgctccagtctaaatatgttgaagagcagcctggtcatttacaaatgggctttgcttccatccgcactgcaactggttgttacattggctggtgcaagggtgtttatgtctttgtgaaaaatgggataatggatacagtacaatttggaaaagatgcttatgtctatctgaagaatcctcctcgagattttcttccgaaaatgggagttattacagtttcaggattggcgggcttggtttcagcgagaaaaggttccaagtttaagaaaattacttatcctctgggactggccactttaggagcaactgtttgctacccagttcagtccgtaataattgctaaggtaacagcaaaaaaggtatatgctacaagccagcaaatttttggagcagttaaatcattgtggacaaaaagcagcaaagaagagtcactccctaaacctaaagaaaaaactaagctaggatcctcttccgaaatagaagtacctgcaaaaacaactcacgtcttgaaacactcagtgcccttgccaacagaactcagctctgaagcaaagaccaaatcagaatccacttcaggtgcaacccagtttatgcctgaccccaagctcatggatcacgggcagtcccacccagaagatatagatatgtacagcactagaagctgaagtagactgcatgcagagactacacagaaaactacaagatgtgtggcgttgcaaataatgatgaaaataaatcatgtaatgggtaactgatacatagagtattacctaaaccaagtttctcccttacatttccaaatatgccatttgataaattacataagtggttaacacacaaactgtgaatgcagtgtaaacaatattaaatgattgatgaggagacttaggtagaagtattagcttgcatttgatgacctttaagggcacttaaaaaaaaatgaagacatgaattctagtgagattaaaatctaaaaatacatatatatatatgtatgtataaatacatacatatttatacatgtataaatacgtatttatacatatatgtataaatacatatacatatatgtataaatacatatacatatttatacatatatgtataaatacatacatatttatacatatatgtataaatacatacatatttatacatatatgtataaatacatacatatttatacatatatatgtataaatacatacatatttatacatataaacatatatttctgctctagctttcccagattaatgctagttaaattagaatggtgtatggaaaggtgtgtttccctctgtttttttttttttttttttttttttttttcccatttcctagagctggaataaaatatccatgttttatgggacttgagcttggctttgtcccttggttttgttaatgttaattcctcttaaatatctaaaaacatctctacctatttgagagggccatatgctacacaggcttcctaatactgcttagcaatccttctactggttcctaagtggtttactattgtgttccctggaagaggtattacaaacgttttattcttctcaagcctcttagaactgtcaaagttgactgtgcctcctatttcatagagaaaaaactaaattattagatatgaccattcccaaaacttcttcccatctaccttcaagacttacctcttttcagccatgcatcccccttttctaagacatgcttctctttgtgttcttgacctcattccttcccatctaagaccatattccaccctgtttaatttctagtcttcggtttctctttttacagtgagcaaattagaagaataggttagaagaatagggtagtagatactgtagtttctttcctccacctctacatagctgcaggtcttctctgtcttaaatcaacaataacaaaacaagacaaaaacctttccttaacttggcctcttcccttgaactattttttctctctccttcctataattgttatacttctttaaaaagtaattgttactttttgacaaaagtaaatattgtcccttttttccctactttcaagtcctctctgtcttgtgacattctggcctccatacctgcttaattttttttgagatggggtcttgctctgttgcccaggctggagtgcagtggcacaaacacagctcactgcaacctccacctcccaggatcaagcaatcctcccatctcaccctcccaagtagctgggactataggcacccgccaccatgcctaattttttgtatttttggtagtgacagggtttcaccatgttgtccaggctggtctcgaactcctgagctcaagcatctgtccgactcagcctaccacagtgttgggattacaggcgtgagccaccgcacccagcccctacttgaatttttatgtcattattgactcttcaccactctctccttgagactcacttatccctttgctctctatttttcctgcttttgttattcttcatttttcttctcttgctctgaatcccttaaatgctgaggctgctcaagtttcatccctagtcctattttctcattctatatgcacactcatttttcatttaacagacttcactggcttcaggcaccatttgttttattattcatattttcaacccccatatctctctagtgctgtagacctttatttctacttactagttatattcacctaacatcccatagatatgccaatgtcaatgtgcctaaaacaaaattgtctctacctcccgctccttcaaatctattctgtcttctctaatcctataattactctccctcaagtcatcatcatcacttttctgggttactgtaatagccttcaggctaatttgctcactgtcaatcttgctgtctctcatttcagaatatgttctgcttgcaggtgacagtaaactagttaaacaaatatttaatattagcttaagcaaaaaaaaaaaaaaaggcctatcctatttatagcctagaattaggcagtatagggttgggacagtggctcattggtgctatcaaggacgtagactttttctctttccactctgtcctctacagcatgctggtttgtcacctcatggtcacaagatgtctgctgtacctccagggagcagatacacattcaaagtaggaaatagcagataaaaatgtacacaagatgcgtctgactcccttttaccagaaaagtaaatgctttcttagaatctcctcagaagacctctgtgtatatctcaaccagtacttgtcatatagccacacacttagctcaaggaagatagggaatagggaaatcatgtttcttttcccctgtggtgaggaaggcaaggggtaaggggttgggaacaggtgctgaattaaagctaacatacagtatctactaccctattcttctaacccatttgtctattatgatatagttgtcttgacaactataatgaatagctgtctttctgaaatctttagtggctcatcattgcctgcagaatcaaaactccttagtctgatatacaaggcccttcatatctgattccagcttaccttctaatcctatcttccctctccttacccaccctactgctaatgtgtattatattgtctttatgacataccatttgtttcttgaacatgctttgctcattattgcctctttctctgcctgtgctagtgtgtcctcttagaatgttcttccatcttatcctgctctacattctaacttctacccatcctacaagtccaattccagtgtcatgttccctttctcaatcctccagggtagaatttagatgttctgtcttttagagataggtattgctatgttgcccaggctggtctcaaactcatggcctcaagcgatcctcccacctcagccaaccagacatgagtgactgtgctcagccttttttaaaaaaatgaaggtatttggtatggccgggtatggtggctcacacctataatcccagcactttgggaggccaaggtgggtgaatcacctgaggtcaggagtttgagaccagcctgaccaacatagtgaaaccctgtctctactaaaaacacgcaaaaagtgcaaaattagctaggcgtggtggctcatgcctgtaattccagctacttgggaggctgaggcaggagaatcacttgaatccgggaggtggaggctgcagtgagccgagatcgtgccattgcacttcagcctgggcacaagaacaaaactcagtctcaaaaaataaataataaaaaataaaggtatataatatgtcttagatttgatgaaatatgagtgtgtttgagatctgatccaaggttttaaaaaattcatttcaaatcctggagataaattctgaggagcttacttatgaaaaactagcaagtgatagctggtatttacagatatggtactttacacaatttatgtaattaatattttctcacagacaaggttccttgtggatatcactgaataatttttgtgtagtttctatgcccatgaagtgcctggtaggtagtaggaactcacacgttctgctatgctggttgcagcacaggacacaaaagttagaattatttcaaaggacttatactctactaggaaagagagaaacatagaaatatagtgttgtatgtggtatcttccctaagataagtacaaccaaagagctatggatgttcagagatagaatgtagtactgggatgatgtcaaggaggcagtggtacttaagctgctcaaggtagggaagcagtttacttgctagtttttcataagtaagctcttcagaatttatctccagtatttgaaatgaatttttttaaacctcagatctgaatatattcaaacacccatatttcatcaaatctaaggcatatcatatacctttaatttaaaaaaaaatgctgagcagagttgcttatgtctgggaggctgaagtgggaggattgcttgaggccaaaagtttgagaccagcctgagcaacatagcaagatcctgcctctaaaaacaaacaaacaaaaaatgctgccagttgatcattacatttatcccgacatcagatatgtcaaattgtgaaaagcaccttaattttgatgaaatatgataggtaaaaaatagcgcaaagctaagtataagtgacttttaaattatctgctcttttggattatcactactgctactagccgttagaatattggtttggataatggaaagttgactagagctacaaaatctgtttcaaaataaaagatctgctcaaacaagaatgtgatgatcacttgctgtattgtacaacaaagtcaaactgctgttagactcaggtgacagtcaaatgttggaggctgtgttgggatgagttgttacaaagcctctttcattcttcccatgccatgtccaggagttcttctctgttaaacctgaagagatgccagccctcctcagaggaaaggtctaaagcccctcgactagtatagtagttgatagaagcctggttccaaatgacgacaagaaagtgcatgcagttacattgagttgtgatggctatctgtttaaaaacaaacaagcaaatattactttcatttgaaagtataatcttcgttaacacatatattttattgaaggtctactctatgcaagacactatgtgtgaataatacttccaaatatagtagttaccttttgaacttttctatagctgtaaaaagaaaaaagtctcactgttctgagctttaaagccctctttaaaatctcaaaactgatttgcaaagcctttatcatacagaaaaggtgtacctaaaagaacacatgttctcatatgtttccaaaatacaagaagatcttgcttttatagtcttgtaatattcatactgtgagcctgtgtttttgaataaaaatgctttctaatctccctccttcttactttcccttgatagcaaaaagggtgtatagtgtggttgtggattatatataaatcaattttaattgactataattcatatactgtatatgatcatctgatttgaaaatccagtttaatttgggtagaaatttgaagtatatgttggggttatgttcaaatggtgagattaatgatttaatttatgccctgtgaatatttattaagaaaaggttaatgtatagaatatcaatactaattgtaatacatacctgacttagcctcttcagcatttcagctccaatacctaggccttttaaatagacaatgcaaagtgaaaactgcaaatttcacttaacttgttatttgctttagtttaaaataaataatatctaaaactctg

SEQ ID NO: 62 - protein, 268 aa, Homo sapiens
MAAIRMGKLTTMPAGLIYASVSVHAAKQEESKKQLVKPEQLPIYTAPPLQSKYVEEQPGHLQMGFASIRTATGCYIGWCKGVYVFVKNGIMDTVQFGKDAYVYLKNPPRDFLPKMGVITVSGLAGLVSARKGSKFKKITYPLGLATLGATVCYPVQSVIIAKVTAKKVYATSQQIFGAVKSLWTKSSKEESLPKPKEKTKLGSSSEIEVPAKTTHVLKHSVPLPTELSSEAKTKSESTSGATQFMPDPKLMDHGQSHPEDIDMYSTRS

SEQ ID NO: 63 - DNA, 14563 bp, Homo sapiens
  source 1..14563: mol_type = unassigned DNA
gcagacgccggcccgagagggctgcgaggggcaacttcttagagtggcccatcggtcggtctagggaggggagggtcagcgtggcaaggtgtgtgtcgggggcagagggaaggagggaaggagtgctggggagaaggggcagtagtgtcggggtaggaagggacagttgttggggagaagaggcagttgtggagggagagggagcaaggaatatttgtgtgggggagggggaggagaaggggcagttgtgacagctgggggggaagagccattctgaggggaaatttgcctgctggtaaccagtttaaggataccagctgctggtataaatactgctggataaatactgctggatttatactgcaggataaatactgctggcccctgcggtatttcctagtactagagtgagccccgacttagcagagcagttcctcctggggcctgcggtgtgggatcgcgtggtgaaccccacggtgcatgcgcctcaggctctagtttgaggcaggaaagcgcagcttgatgcttctctggagactgatggaggaagcctccttgctgtagttggttttattgatttgctggcctaacagaacgtttttccttggagcaaagtacaaatccttcaagtttgaaattcataacctgagatcaatgcctgtggcagcctgtggggatgaggaaggagagccacaggtgccgttaggctgcagcctaatgaaaagagagtgcccagcgcctcagactttgcgcctgggattctgagcacctgtccgagatccccgcttcctgccatcctacctttctgagagaggcaccactgtgaccttcctcgtgccccaattgctttttcctcatgacacaatttgaaatttatgattggatgacttttgtccttctttcttccaatctgttctcagggcattttgagtcaaataaatgatcctgactgatcttaaccattagcacagagttcctcagccaactctgctaagagacctcagtacacacaaaacagtgttcctgcccctcaggacttcaaagcgatagacccaggttttgcctgcacgatgtccagcaaagctgagaagaagcagcgattgagtggccgaggaagctcccaggcaagctggtcagggcgggccactcgggctgctgtggccacacaggagcaggggaatgccccggctgtcagtgagccagagctgcaggctgagctccccaaggaggagcctgagccacggttggagggacctcaagcacagagtgaagaatcagtggagcccgaggcagatgtgaagcccctcttcctttcccgagctgcgctgacaggactggcggatgcagtgtggacacaggagcatgatgccattctggaacactttgcccaggaccctacagaatccatcctcaccatcttcattgacccttgttttgggctgaagctagagctgggcatgcctgtacagacccagaaccagcttgtctacttcattcgccaagcaccagttcccatcacctgggagaacttcgaggcaactgtgcagtttgggacggtgcggggcccctatatcccggccctgcttcggctgctcggtggagtctttgcccctcagatctttgcaaacacaggctggcctgagagcattagaaatcattttgcttctcatctgcacaagttcttggcctgcctgacagacactcggtacaaactggaggggcacacggtcctctacatccctgcagaggccatgaacatgaagcctgagatggtgataaaggacaaagagctggtgcaacggctagagacctccatgatccactggacccggcagataaaggagatgctcagtgcccaggagactgtggagacaggagaaaatttaggtcctctggaggagattgagttctggcgcaaccgatgcatggacctgtctggcatcagtaagcagctggtgaagaagggagtgaagcacgttgaatccatcctgcaccttgccaagtcgtcctacttggcgccctttatgaaactggcacagcagatccaggatggctctcgtcaagcacagtcaaacctgacctttttgtcaatcctgaaggaaccttaccaggagttggctttcatgaagcccaaggacatctctagcaagctccctaagctgatcagtctcatccgcatcatctgggtcaactctccccactacaacactcgggagagactgacctcgctcttccgaaaggtatgtgactgtcagtatcacttcgcccgctgggaagatggcaagcagggtccccttccttgcttctttggtgcccaggggccacagataacacggaacttgctggagattgaggacatctttcataaaaatctgcacacgctgcgagccgttcgcgggggtatcctggatgtcaagaacacctgttggcatgaagactacaataagttccgtgccggaatcaaggacctggaggtgatgacccagaacctgatcacctcagccttcgagttggtgcgggacgtgccgcacggcgtgcttctgctggacaccttccacaggcttgcctcccgcgaggctatcaagcggacttatgacaagaaggcggtggatctctacatgctgttcaatagcgagctggccctggtgaaccgtgaacggaacaagaaatggccagacctggagccctacgtggcccagtattccggaaaggcgcgctgggtgcacatcctccggcgtcgcatcgacagagtcatgacctgccttgctggtgctcatttcctgccccgtattgggactggaaaggagagtgtgcacacctatcagcagatggtccaggccattgatgagctggttcgaaaaaccttccaagagtggacatcaagtctggacaaggattgcattcggcggttggataccccattgctgcgaatcagccaggagaaggcgggcatgctggatgtcaactttgacaagtcccttctgattctctttgcggaaattgactactgggagcggctgctgtttgagacgccccattacgtggtgaacgtagctgagcgagccgaggacctgcgcattctgcgtgaaaatctgctactcgttgctagagactacaataggattattgccatgctgtccccagatgagcaggccctattcaaagagcgtattcggctcctggataagaagatccacccgggactcaagaaactgcactgggccttgaagggggccagtgccttcttcatcacggagtgccgtatacatgccagcaaggtgcagatgattgtgaatgagttcaaggcatccactctgaccattggctggcgagcccaagagatgtcagagaagctgctggtacgcattagtggcaaacgggtatacagggacctggaatttgaagaggaccaaagagagcatcgggcagctgtacagcagaaattgatgaacctgcaccaggatgtggtgaccatcatgaccaactcctatgaggtcttcaagaatgatggtcctgagattcagcagcagtggatgctgtacatgattcggctggaccgcatgatggaggatgccctgcgcctgaatgtgaagtggtcactgctagaactatccaaggctatcaacggggatggaaagaccagcccaaacccactcttccaagtccttgtcattttgaagaatgatctgcaaggaagtgtggcacaggtggaattctcacccactctgcagactttggcaggtgtggtcaatgacattggcaaccacctcttttccaccatctctgtcttctgccacctccctgacattctcaccaagcgcaagttacatcgtgaacccatccaaacagttgtggagcaagatgaggacatcaagaagatccagacccaaatcagcagcggcatgactaacaacgcaagcctgctgcagaactacctcaagacctgggacatgtaccgggagatctgggagatcaacaaggactccttcattcatcgctaccagcgcctcaaccctcctgtctcttcttttgttgccgacattgcccgctacacggaagttgctaataacgtgcagaaggaggagacagtcaccaacatccagtttgtgctgctggactgttcgcacctcaagttctccctggtgcagcactgcaatgaatggcagaacaagttcgcgactctgctcagggagatggctgctgggcgcctcctggagctgcacacctacctgaaggagaacgcagagaaaatcagccgccctccgcagacactggaggaactgggggtcagcttgcagctcgtggatgccctgaagcacgacttggccaacgtggagactcagatccctcccatacacgagcaatttgccattcttgaaaagtacgaggtgccagtcgaggacagtgtcctggagatgctggacagtctcaacggggagtgggttgtcttccaacaaactctgctggacagtaagcaaatgctgaagaaacacaaggagaaattcaagacaggcctgatccactcggcagatgacttcaagaagaaagcacatacacttctggaagatttcgaattcaaaggccatttcaccagcaacgtgggatacatgtctgccttagaccagattacacaagtgcgggccatgctgatggccatgcgggaagaggaaaatagtctccgagccaacctgggcatcttcaagatcgagcagccaccctccaaggaccttcagaacctggagaaggagctcgatgccctccagcaaatctgggagatcgcacgagactgggaggagaactggaatgagtggaagactggccggttcctgatcctgcagacggaaaccatggagaccacggcccacgggctgtttcgtcgcctcacaaaattagccaaagagtataaggaccgaaactgggaaattattgaaaccactcgctcaaaaatagagcagttcaagaggaccatgcctctcatctcagacctgcggaaccctgcccttagagagaggcactgggaccaggtccgggatgagatccagcgggagtttgatcaggaatctgaaagcttcaccttggagcagattgtggagcttgggatggatcagcatgtggagaaaattggggagatctctgcttcagcaactaaagagctggctatagaagtggctttacaaaacattgccaagacctgggatgtgactcagctcgacatagtaccctacaaggataagggccatcatcggctcagaggtacagaagaagtattccaggcactggaagataaccaggtagctctgtctaccatgaaggcatcacgctttgtcaaggcctttgagaaggatgtggaccactgggaacgctgcctctccctcattttggaggttattgagatgattctcacagtgcagcgtcagtggatgtacttagagaatatcttcctaggagaagacatccgcaagcagctgcccaatgaatcgaccttatttgaccaggtcaacagcaactggaaagccatcatggacaggatgaacaaggacaacaatgctctccggagcacccatcacccaggcctcctggacacattgatagaaatgaatacaatcctggaagatattcagaaatctctggatatgtatttagagaccaagcgacatattttcccccgcttctacttcttgtccaatgatgacctgctggagattctgggccagtcccgaaacccagaggctgtgcagccacacctcaaaaaatgctttgacaacatcaagttgctgagaatccagaaggttggagggcccagcagcaaatgggaagctgtggggatgttctcgggcgacggcgagtacattgacttcctccactcagtatttttagaaggccctgtggagtcctggcttggcgatgtggaacagaccatgagggtgaccctgcgggaccttctccggaactgccacctggccctcaggaagttcctcaacaagagggacaaatgggtgaaggagtgggctggccaggtggtgatcactgccagtcagatccagtggacggctgatgtcaccaagtgcctgctgacagcgaaggagcgggcagacaagaaaatcctcaaggtcatgaagaagaaccaggtgtcaatcctgaataagtattcagaagccatcagggggaacttgaccaagatcatgcggcttaaaattgtggctctggtgacgatagaaattcatgcccgggatgtgttggagaagctttacaagagtggcctcatggatgtcaattcctttgactggctcagccaacttcggttctactgggagaaggatcttgatgactgtgtcatccgccagaccaacacgcaatttcagtataattatgagtacttgggtaactcgggccggctcgtcatcacccccctgacggacaggtgttacatgacactgaccacggcattgcacctgcaccgagggggctcccccaaaggccctgcaggcacaggcaagaccgagaccgtcaaggacctgggcaaggccctgggcatatatgtcattgtggtcaactgctctgagggcctggactacaagtccatgggccgaatgtactcaggtctggcccagactggagcttggggctgctttgatgagtttaaccgcatcaacatcgaggtgctgtcagtggtggcccaccagatcctgtgcatcctgtctgccctggctgccggcctcacccatttccattttgatggctttgaaataaatctggtgtggtcctgtgggatcttcattaccatgaatcctggctatgctggccgcacagagcttcccgaaaatcttaaatccatgttccgcccaattgccatggtggtgcctgactccaccctcattgcagaaatcattctctttggagagggctttggcaactgcaagattctggccaagaaggtgtacacactctactcactggctgtgcagcagctgtccagacaggaccactatgactttggcctgcgtgccctcacctcccttctgcgctatgctggcaagaagcgccgcctacagccggatctgactgatgaagaggttctgctgctctcaatgagagatatgaacatcgccaagctcacttcagttgatgcacccctgttcaatgccatcgtgcaagatctgtttcccaacattgagctgcctgtcattgactatggcaagctgcgggagaccgttgagcaggagattcgagacatgggcctgcaaagcacgccgttcaccctcaccaaggttttccagttgtatgaaaccaagaactcccgccactccaccatgatcgtgggctgcacgggcagcggcaagactgcctcatggcgcattctacaggcctccctgtcctctctgtgccgcgccggagaccctaacttcaacattgttagagagttccctttgaaccccaaggcattgtccctaggggaactgtatggggaatatgacctcagcaccaatgaatggacagatggcatcttgtccagtgtcatgcggacggcatgtgcagatgagaaacccgacgagaagtggatcctgttcgatggccccgtggacacactgtggatcgagaacatgaactccgtcatggacgataacaaggtgttgaccctcatcaacggcgagcgcatcgcgatgcccgagcaggtgtctctcctgtttgaagtggaggacctggcaatggcctctccggccactgtatcccgctgcgggatggtctacactgactacgctgacctgggctggaagccctatgttcagtcatggctggagaagaggccaaaggctgaggtggagccccttcaacgcatgttcgaaaagctcatcaacaagatgctggcctttaagaaggacaactgcaaggagctggtgcccctgcccgagtacagcggtatcacctccctctgcaagctgtactctgccctggccacgccagagaatggggtgaacccagctgacggcgagaactatgtcaccatggtagagatgacatttgtgttcagcatgatctggtctgtgtgtgcctctgtggatgaggagggccggaagaggatcgacagctacctccgagagatcgagggctcctttcccaataaggacacggtatatgagtattttgtggaccccaaaatacggagttggacatcatttgaggacaagctccctaagagttggcgctaccctccaaacgcccccttctataagatcatggtgcccaccgtcgacactgttcgctacaactacctggtgagcagcttggtggccaaccagaatcccattctgctggtgggtcccgtggggactgggaagacctccatcgcccagagcgttctgcagtccctgccctccagccagtggtcggtgctcgttgtcaacatgtccgcacagaccacatccaataacgtgcagagcatcattgagagcagggttgagaagcgaaccaagggtgtctacgtgccattcgggggcaaaagcatgatcacctttatggatgacctaaatatgcccgctaaggacatgtttgggtcccagccacccctggagctgatccgcctctggattgactatggcttctggtatgaccgtacgaagcagaccatcaagtacattcgagaaatgttcctgatggctgccatgggcccccctgggggtggacggactgtcatctccccaaggctacggagtcgcttcaacattatcaacatgaccttccccacaaagtcccagatcatccgcatattcggcaccatgatcaatcagaagcttcaggactttgaggaagaggtgaagcccattgggaacgtggtgacagaggccaccctggacatgtacaacaccgtggtacagcgcttcctgcccacgcccaccaagatgcattacctcttcaaccttcgagacatctccaaggtgttccagggcatgcttagagccaacaaggacttccatgataccaagtccagcatcacacggctctggatccatgaatgtttcagagtcttctctgaccggctggttgatgcggcagacacagaagccttcatgggcatcataagcgacaagctcggctccttctttgacctcacatttcatcatctctgtcccagcaagcgtcctcctatctttggggatttcctgaaggagcccaaggtgtatgaagacctcacggatctgacagtgctgaagacagtcatggagacagctctaaatgagtataacctgtcaccctctgtcgtgcccatgcagctagtgctcttccgagaggctattgaacacatcacacggatcgtgcgggtcattggacagcctcggggcaacatgctcctggtgggtatcgggggcagcggacgccagagtctggcccgcctggcttcatccatctgcgactacaccaccttccagatcgaggtcaccaaacattatcggaagcaggagttccgagatgatatcaagcgtctgtatcgccaggctggggtggagctcaagaccacgtccttcatttttgtggacacccaaatagctgatgagtccttcctagaggacatcaacaacatcctcagctcaggcgaggtgcccaatctctacaagcctgatgaatttgaagagatccagtcgcatatcatagaccaggcccgggtggagcaggtgcctgagtcatcggacagcctcttcgcctacctcattgaacgcgtgcagaacaacctgcacatcgtgctctgcctcagccccatgggggatcccttcaggaactggatccgccagtacccagccttggtgaactgcacaaccatcaactggttctcagagtggccccaagaggccctgctcgaggtggctgagaagtgcctcataggagtagacctgggaactcaggagaatatccacaggaaggtggcccagatctttgtcactatgcactggtcagtagctcagtattcccagaagatgctgttggaactgcggagacacaactatgtcacacccaccaaatacctggaactcctgtctggatataagaagttgctgggagaaaaacggcaggagctgctggcccaagccaataaactgcggacaggcttgttcaagatcgacgaaactagggaaaaggtgcaagtgatgtcgttggagctggaggatgccaagaagaaggtggctgagttccagaagcagtgtgaggagtacctggtcatcattgtgcagcagaagcgggaggcagatgagcagcagaaggccgtaacagccaacagtgaaaagattgcagttgaggaaatcaagtgtcaggcactggctgacaatgcccagaaagatctagaagaggcactgcccgccctggaagaggccatgcgggccctggagtctctgaacaagaaggatataggagagatcaagtcttatggacggcccccagcccaagtggagatagtgatgcaggcagttatgattcttcgaggcaacgagcccacatgggcagaggccaagaggcagctaggggaacagaacttcatcaagtcactgatcaactttgataaagacaatatctcagataaggttctgaagaagattggggcctactgcgcccagcctgacttccagcctgatatcatcggccgcgtctccctggctgccaagtccctctgcatgtgggtgcgggccatggagctgtatgggcggctatatcgggtggtggagcccaagcgaatccgaatgaacgctgccttggctcagcttcgggagaagcaagccgcgctcgctgaggcccaggagaagctgcgggaggtagctgagaaactggagatgctaaagaaacagtatgatgagaagctggcacagaaggaggagcttcgcaagaagtctgaagagatggagctgaagctggagcgagctgggatgctcgtgtcggggttggctggcgagaaggccagatgggaggagacagtccagggcctggaggaggacctgggctacctggtgggggactgtctcctggcagctgccttcctgtcctacatgggacccttcctgaccaactaccgggatgagattgtcaaccaaatctggatcgggaagatctgggagcttcaggttccttgctccccttctttcgccatcgataacttcctgtgcaatcctaccaaagtccgggactggaacatccaagggttgccctcagacgccttctccactgagaatggcatcatcgtcacccgaggcaacaggtgggcactgatgatcgaccctcaggcccaggccctgaaatggattaagaacatggaaggaggccagggcctgaagatcatcgacctgcagatgagcgattacctgcgaatcctagaacacgccattcactttggatacccggtgctacttcagaacgtgcaggaatatctggaccccacactgaaccccatgctcaacaaatctgtagcccgaatcggtggtcggctgttgatgcgcattggcgataaggaggtggaatataataccaatttccgtttctacatcaccaccaagctctccaacccccactacagcccagagacctcagccaagaccaccatcgtcaactttgctgttaaagaacagggcctggaggcccagctgctgggcattgtggtgcggaaggagcggcctgagctggaggagcagaaggactcactggtcatcaacatcgcggctggtaaaaggaagctcaaggagctggaggatgagatcctgcggctgctgaatgaggccaccggctccctgctggatgatgtgcagctggtgaacacgctgcatacctccaagatcacagccacagaggtgactgagcagctggagaccagtgagaccacagagatcaacactgacttggcgcgggaggcttaccgcccatgcgcccagcgggcatcaatcctgttcttcgtgctcaatgatatgggctgcatcgaccccatgtaccagttctcactggatgcctacatcagcctctttattctcagcattgacaaaagccaccgcagcaataagctggaggaccgcattgactacctgaatgactaccacacctacgctgtctacaggtacacctgccgtacccttttcgaacgccacaaactactattcagttttcatatgtgtgccaaaatcttggagacttctggcaagctcaacatggatgaatacaacttctttctacgtgggggtgtggtcttggatcgggagggccaaatggacaatccatgtagtagctggcttgcagatgcctactgggataacatcacagagctagacaaactgaccaacttccacggactcatgaactcctttgagcagtaccctcgtgactggcacctgtggtataccaatgctgccccggagaaggcgatgctgccaggtgagtgggaaaatgcctgcaatgaaatgcaacggatgctgatcgttcgctccctgcgccaggaccgcgtggccttctgcgtgacctccttcatcatcaccaaccttggctcccgcttcatcgagccgcctgtgctgaatatgaagtcggtgctggaggattcaaccccacgatccccactcgtgttcatcctgtcccctggtgtggaccccaccagtgccctgctgcagctggcagagcacatgggcatggcccagcgcttccacgccctgtccctgggccagggccaggcccccatcgctgctcggctcctccgagagggtgtgactcagggacactgggtgttcctggcaaactgccacctgtcactgtcttggatgcctaatctggacaagctggtggagcagctgcaggtggaggatcctcatccatccttccgcctctggctcagctccatcccccacccagacttccctatctcaatcttgcaggtcagcatcaagatgaccacagagccaccaaagggcctaaaggccaacatgacacgtctttaccaactgatgtcagaaccacagttttcccgctgctccaaacctgccaaatataagaagctgctgttttcactctgtttcttccactctgtgttacttgaacgcaaaaagttcctgcagcttggctggaacatcatctatggcttcaatgactccgactttgaggtgtcagaaaacttgctgagcctctatctcgatgagtacgaggagacaccttgggacgcacttaagtacctcattgccggcatcaactatggtggacatgtcacagatgactgggaccggcgcctgctgaccacctacatcaatgattatttctgtgaccagtctctatcaactcccttccaccggttgtcagcactggagacttatttcatccccaaggatggcagcctcgcttcttacaaggaatacatcagcttattgcctggcatggacccccctgaggcctttggccagcaccccaatgctgatgtggcctctcagatcactgaggcacaaaccctctttgatactttgctttccttgcaacctcagattacacccaccagggctggaggccagacccgggaagagaaggtccttgagttggccgctgatgtgaagcagaagatccctgaaatgatcgactatgaggggactcaaaaactgctagctctcgacccctcccccctcaatgtggtccttctgcaggagatccagagatacaacacactgatgcagaccatcctgttctcactgacagacctagagaaaggcatccagggtctcatcgtcatgtctacaagcctggaagagattttcaattgcatctttgatgcccatgttcctccgctctggggaaaggcatacccctcacaaaagccattggctgcctggacccgggacttggccatgcgtgtggagcagtttgagctgtgggccagccgggcccggcctcctgtgatcttctggttgtctggtttcacctttcccactggcttcctcactgctgtgctgcagtcttcagctcgccaaaacaacgtttcagtggacagcctctcctgggagtttatcgtttccactgtggatgacagcaacctagtgtatccccccaaggatggtgtctgggtccggggcctgtacctggaaggtgctggctgggaccggaagaactcctgcttggtggaggcagagcccatgcagcttgtctgcctcatgcccacgatccacttccggcctgcagagagccgcaagaagagcgccaagggcatgtactcctgcccctgctattactatcccaaccgggcaggcagctcagaccgagcctcctttgtcatcggcattgacctgcggtctggggccatgacacctgatcattggatcaagaggggcactgctctactcatgagcctggacagctgagacctcctcctcttctccgcttgagagagagggtcagggactccaggagctaagacagatgttgcacctaggactgaggccggacctcactcagactttgaccttggccgaatttgtgtgatgtggccctggagatacctagttgtgttagccataaaagtgaaagagttgtattggagctcagtgctgtaaaacacccgcgacaacaa

SEQ ID NO: 64 - protein, 4427 aa, Homo sapiens
MSSKAEKKQRLSGRGSSQASWSGRATRAAVATQEQGNAPAVSEPELQAELPKEEPEPRLEGPQAQSEESVEPEADVKPLFLSRAALTGLADAVWTQEHDAILEHFAQDPTESILTIFIDPCFGLKLELGMPVQTQNQLVYFIRQAPVPITWENFEATVQFGTVRGPYIPALLRLLGGVFAPQIFANTGWPESIRNHFASHLHKFLACLTDTRYKLEGHTVLYIPAEAMNMKPEMVIKDKELVQRLETSMIHWTRQIKEMLSAQETVETGENLGPLEEIEFWRNRCMDLSGISKQLVKKGVKHVESILHLAKSSYLAPFMKLAQQIQDGSRQAQSNLTFLSILKEPYQELAFMKPKDISSKLPKLISLIRIIWVNSPHYNTRERLTSLFRKVCDCQYHFARWEDGKQGPLPCFFGAQGPQITRNLLEIEDIFHKNLHTLRAVRGGILDVKNTCWHEDYNKFRAGIKDLEVMTQNLITSAFELVRDVPHGVLLLDTFHRLASREAIKRTYDKKAVDLYMLFNSELALVNRERNKKWPDLEPYVAQYSGKARWVHILRRRIDRVMTCLAGAHFLPRIGTGKESVHTYQQMVQAIDELVRKTFQEWTSSLDKDCIRRLDTPLLRISQEKAGMLDVNFDKSLLILFAEIDYWERLLFETPHYVVNVAERAEDLRILRENLLLVARDYNRIIAMLSPDEQALFKERIRLLDKKIHPGLKKLHWALKGASAFFITECRIHASKVQMIVNEFKASTLTIGWRAQEMSEKLLVRISGKRVYRDLEFEEDQREHRAAVQQKLMNLHQDVVTIMTNSYEVFKNDGPEIQQQWMLYMIRLDRMMEDALRLNVKWSLLELSKAINGDGKTSPNPLFQVLVILKNDLQGSVAQVEFSPTLQTLAGVVNDIGNHLFSTISVFCHLPDILTKRKLHREPIQTVVEQDEDIKKIQTQISSGMTNNASLLQNYLKTWDMYREIWEINKDSFIHRYQRLNPPVSSFVADIARYTEVANNVQKEETVTNIQFVLLDCSHLKFSLVQHCNEWQNKFATLLREMAAGRLLELHTYLKENAEKISRPPQTLEELGVSLQLVDALKHDLANVETQIPPIHEQFAILEKYEVPVEDSVLEMLDSLNGEWVVFQQTLLDSKQMLKKHKEKFKTGLIHSADDFKKKAHTLLEDFEFKGHFTSNVGYMSALDQITQVRAMLMAMREEENSLRANLGIFKIEQPPSKDLQNLEKELDALQQIWEIARDWEENWNEWKTGRFLILQTETMETTAHGLFRRLTKLAKEYKDRNWEIIETTRSKIEQFKRTMPLISDLRNPALRERHWDQVRDEIQREFDQESESFTLEQIVELGMDQHVEKIGEISASATKELAIEVALQNIAKTWDVTQLDIVPYKDKGHHRLRGTEEVFQALEDNQVALSTMKASRFVKAFEKDVDHWERCLSLILEVIEMILTVQRQWMYLENIFLGEDIRKQLPNESTLFDQVNSNWKAIMDRMNKDNNALRSTHHPGLLDTLIEMNTILEDIQKSLDMYLETKRHIFPRFYFLSNDDLLEILGQSRNPEAVQPHLKKCFDNIKLLRIQKVGGPSSKWEAVGMFSGDGEYIDFLHSVFLEGPVESWLGDVEQTMRVTLRDLLRNCHLALRKFLNKRDKWVKEWAGQVVITASQIQWTADVTKCLLTAKERADKKILKVMKKNQVSILNKYSEAIRGNLTKIMRLKIVALVTIEIHARDVLEKLYKSGLMDVNSFDWLSQLRFYWEKDLDDCVIRQTNTQFQYNYEYLGNSGRLVITPLTDRCYMTLTTALHLHRGGSPKGPAGTGKTETVKDLGKALGIYVIVVNCSEGLDYKSMGRMYSGLAQTGAWGCFDEFNRINIEVLSVVAHQILCILSALAAGLTHFHFDGFEINLVWSCGIFITMNPGYAGRTELPENLKSMFRPIAMVVPDSTLIAEIILFGEGFGNCKILAKKVYTLYSLAVQQLSRQDHYDFGLRALTSLLRYAGKKRRLQPDLTDEEVLLLSMRDMNIAKLTSVDAPLFNAIVQDLFPNIELPVIDYGKLRETVEQEIRDMGLQSTPFTLTKVFQLYETKNSRHSTMIVGCTGSGKTASWRILQASLSSLCRAGDPNFNIVREFPLNPKALSLGELYGEYDLSTNEWTDGILSSVMRTACADEKPDEKWILFDGPVDTLWIENMNSVMDDNKVLTLINGERIAMPEQVSLLFEVEDLAMASPATVSRCGMVYTDYADLGWKPYVQSWLEKRPKAEVEPLQRMFEKLINKMLAFKKDNCKELVPLPEYSGITSLCKLYSALATPENGVNPADGENYVTMVEMTFVFSMIWSVCASVDEEGRKRIDSYLREIEGSFPNKDTVYEYFVDPKIRSWTSFEDKLPKSWRYPPNAPFYKIMVPTVDTVRYNYLVSSLVANQNPILLVGPVGTGKTSIAQSVLQSLPSSQWSVLVVNMSAQTTSNNVQSIIESRVEKRTKGVYVPFGGKSMITFMDDLNMPAKDMFGSQPPLELIRLWIDYGFWYDRTKQTIKYIREMFLMAAMGPPGGGRTVISPRLRSRFNIINMTFPTKSQIIRIFGTMINQKLQDFEEEVKPIGNVVTEATLDMYNTVVQRFLPTPTKMHYLFNLRDISKVFQGMLRANKDFHDTKSSITRLWIHECFRVFSDRLVDAADTEAFMGIISDKLGSFFDLTFHHLCPSKRPPIFGDFLKEPKVYEDLTDLTVLKTVMETALNEYNLSPSVVPMQLVLFREAIEHITRIVRVIGQPRGNMLLVGIGGSGRQSLARLASSICDYTTFQIEVTKHYRKQEFRDDIKRLYRQAGVELKTTSFIFVDTQIADESFLEDINNILSSGEVPNLYKPDEFEEIQSHIIDQARVEQVPESSDSLFAYLIERVQNNLHIVLCLSPMGDPFRNWIRQYPALVNCTTINWFSEWPQEALLEVAEKCLIGVDLGTQENIHRKVAQIFVTMHWSVAQYSQKMLLELRRHNYVTPTKYLELLSGYKKLLGEKRQELLAQANKLRTGLFKIDETREKVQVMSLELEDAKKKVAEFQKQCEEYLVIIVQQKREADEQQKAVTANSEKIAVEEIKCQALADNAQKDLEEALPALEEAMRALESLNKKDIGEIKSYGRPPAQVEIVMQAVMILRGNEPTWAEAKRQLGEQNFIKSLINFDKDNISDKVLKKIGAYCAQPDFQPDIIGRVSLAAKSLCMWVRAMELYGRLYRVVEPKRIRMNAALAQLREKQAALAEAQEKLREVAEKLEMLKKQYDEKLAQKEELRKKSEEMELKLERAGMLVSGLAGEKARWEETVQGLEEDLGYLVGDCLLAAAFLSYMGPFLTNYRDEIVNQIWIGKIWELQVPCSPSFAIDNFLCNPTKVRDWNIQGLPSDAFSTENGIIVTRGNRWALMIDPQAQALKWIKNMEGGQGLKIIDLQMSDYLRILEHAIHFGYPVLLQNVQEYLDPTLNPMLNKSVARIGGRLLMRIGDKEVEYNTNFRFYITTKLSNPHYSPETSAKTTIVNFAVKEQGLEAQLLGIVVRKERPELEEQKDSLVINIAAGKRKLKELEDEILRLLNEATGSLLDDVQLVNTLHTSKITATEVTEQLETSETTEINTDLAREAYRPCAQRASILFFVLNDMGCIDPMYQFSLDAYISLFILSIDKSHRSNKLEDRIDYLNDYHTYAVYRYTCRTLFERHKLLFSFHMCAKILETSGKLNMDEYNFFLRGGVVLDREGQMDNPCSSWLADAYWDNITELDKLTNFHGLMNSFEQYPRDWHLWYTNAAPEKAMLPGEWENACNEMQRMLIVRSLRQDRVAFCVTSFIITNLGSRFIEPPVLNMKSVLEDSTPRSPLVFILSPGVDPTSALLQLAEHMGMAQRFHALSLGQGQAPIAARLLREGVTQGHWVFLANCHLSLSWMPNLDKLVEQLQVEDPHPSFRLWLSSIPHPDFPISILQVSIKMTTEPPKGLKANMTRLYQLMSEPQFSRCSKPAKYKKLLFSLCFFHSVLLERKKFLQLGWNIIYGFNDSDFEVSENLLSLYLDEYEETPWDALKYLIAGINYGGHVTDDWDRRLLTTYINDYFCDQSLSTPFHRLSALETYFIPKDGSLASYKEYISLLPGMDPPEAFGQHPNADVASQITEAQTLFDTLLSLQPQITPTRAGGQTREEKVLELAADVKQKIPEMIDYEGTQKLLALDPSPLNVVLLQEIQRYNTLMQTILFSLTDLEKGIQGLIVMSTSLEEIFNCIFDAHVPPLWGKAYPSQKPLAAWTRDLAMRVEQFELWASRARPPVIFWLSGFTFPTGFLTAVLQSSARQNNVSVDSLSWEFIVSTVDDSNLVYPPKDGVWVRGLYLEGAGWDRKNSCLVEAEPMQLVCLMPTIHFRPAESRKKSAKGMYSCPCYYYPNRAGSSDRASFVIGIDLRSGAMTPDHWIKRGTALLMSLDS

SEQ ID NO: 65 - DNA, 1205 bp, Homo sapiens
  source 1..1205: mol_type = unassigned DNA
agagcagcgtgtagcagttccctgtggaggattaacacagtggctgaacaccgggaaggaactggcacttggagtccggacatctgaaacttgatctccagcaccctgccggtggcactactgagagacgaggtgccagggtggttcctgaaagtgcctgagccccaacttatcagcaaggagctcatcatgctgacagaagtcatggaggtctggcatggcttagtgatcgcggtggtgtccctcttcctgcaggcctgcttcctcaccgccatcaactacctgctcagcaggcacatggcccacaagagtgaacagatactgaaagcggccagtctccaggttcccaggcccagccctggccaccatcatccacctgctgtcaaagagatgaaggagactcagacagagagagacatcccaatgtctgattccctttacaggcatgacagcgacacaccctcagatagcttggatagctcctgcagttcgcctcctgcctgccaggccacagaggatgtggattacacacaagtcgtcttttctgaccctggagaactaaaaaatgactccccgctggactatgagaacataaaggaaatcacagattatgtcaatgtcaatccagaaagacacaagcccagtttctggtattttgtcaaccctgctctgtctgagccagcggaatatgatcaagtggccatgtgaattccaaatatttttaatggggtccagttctctatggattcttacatttaatttgtagggaaatgccatttttcccccttaaacaaggcatggggctcacaagtctatggagacaggccaaaaagaatgtggagaagaaaactgataaatacacagaggtcctcaagacccatggactcctggtctgtacccaaaaaagctgttcgttcctcaaaaacaaaaacaaggcttggctgggaaaacaggccaatgccccggcaagaaaggttgagatcagatgttaggaagaactttcaggtaaagtatgagaactatggagtccatcagcagagatagtagtgaagtctctccccagggaaaattttaaaaaggttgaatcagctgttgtagagttctatttggcaatctcatggttaaatgacttccctttgagctctttaattattggcaataaacaacttctttaaaagttttaaataaaatagcaaccaccacca

SEQ ID NO: 66 - protein, 172 aa, Homo sapiens
MLTEVMEVWHGLVIAVVSLFLQACFLTAINYLLSRHMAHKSEQILKAASLQVPRPSPGHHHPPAVKEMKETQTERDIPMSDSLYRHDSDTPSDSLDSSCSSPPACQATEDVDYTQVVFSDPGELKNDSPLDYENIKEITDYVNVNPERHKPSFWYFVNPALSEPAEYDQVAM

SEQ ID NO: 67 - DNA, 6330 bp, Homo sapiens
  source 1..6330: mol_type = unassigned DNA
aggccctggagcggacggttcctactgcggctgggcaccggctccgctcccgcgtctgcccgcgctccagctgcgcctggcccggccccggcccggctcggcgtggccccggcctccaagcgaaggcgccgctgccgctgggccgctcccagggccatgaggaagcggcggcagccactgcggcccgcgtcaaggacttctccagacaggttatgttacctgcagaggctgccctgaagctccctgtggcctggagactatgtacaagaggaatggtctgatggctagcgtgttggtcacctctgccactccacagggcagcagcagctcggactctctggagggccagagctgcgactatgccagcaagagctatgatgccgttgtcttcgatgtcttgaaagtgaccccagaggagtttgctagccagattacattaatggatatacctgtgtttaaagctatccagccggaggaactagccagctgtggatggagtaagaaggagaaacacagtcttgcccctaacgttgtggcctttacccggaggtttaaccaggtcagtttttgggttgtacgagaaattctaacagcacagactttaaaaataagggcagaaatcctcagccattttgtgaaaatagccaagaaacttctagaactcaacaaccttcattctctcatgtctgtggtatcagcattacaaagtgctcccatcttcaggctgacaaaaacctgggctcttttaaatcgaaaagacaagactacctttgagaaattggactacctgatgtcgaaagaagataattacaagcggacacgggaatatatccgaagcctgaagatggttccaagtattccctatctaggaatctatcttctggatttaatctacattgattctgcatatcctgcctcaggcagtatcatggaaaatgaacaaagatccaatcagatgaacaatattcttcgaataattgctgatttacaagtttcctgcagctatgatcacctcaccaccctgccccatgtgcagaagtacctgaagtccgtacgctacattgaagagctccagaagtttgtggaagacgacaactacaaactgtcgctcagaatcgaaccaggaagcagctctccaagactagtctcttccaaggaagatcttgcaggtccctctgctggctccggttctgcgaggttcagccggaggcccacctgtcctgacacatctgttgctggcagcctccccacacctccagtccccagacacaggaagagccacagcctaggcaacaatatgatgtgtcagttgagtgtagttgagagtaaaagtgcgacattcccatcggagaaagcaaggcacctactggacgacagtgtcctagagtcccgcagcccccgaaggggcctggctctgacctcctcctctgctgtcaccaatggactctccctaggcagtagtgagagctcagagtttagtgaagagatgtcttcagggctggaaagccccaccggcccgtgcatctgttctctggggaactccgcagctgtgcccaccatggaggggcctctgagaagaaaaaccctgctcaaggaagggcggaagcctgcgctgtcctcgtggaccaggtactgggtcatactctcaggatccaccctcctgtactacggagccaagtccttgcggggcacagacagaaaacactataaatccacacctggcaaaaaggtttccatcgtgggctggatggtgcagctgcccgatgaccccgagcacccagatatcttccagctgaacaaccctgacaaaggcaatgtttacaagtttcagactggttcccgatttcatgcaatactgtggcacaagcatttggatgatgcatgtaaaagcaacaggcctcaggtacctgcaaaccttatgtcatttgagtaagtctctgcaggacgtggcatgacttcagaggcttctgggaacccaggctgggcctggtggtgaagagcagtcctgggcacaggctgtgagccagggtgctgggaaactcacagctggactcaggggacacggcctgtggcctcaccatcccagagggcttcaccagtgtgggatccacctgtcagtccccagcgactctcatgacactcattctgcagcaccgcctcttggggcagtggtcagaccccacacgccctctctgggcccaccacctgcatctgcgactagagagcacccggcccacgttgggttctcagtgctttctactgcacagagtggacagcgctaactaacctgtgagaggggcccgagagaaggaacagctgtggaacaggctttttacaccccaagtgcatggggttgctcgcccacagggctgcctcagattttgtacaaccccgaagcgtcctctgcgtgtgcgtgctgtacgtgtgtgtgtgtgtgtgagcgagtgtgaactcttcaagaaacatgcattttggcacaagactcgtgacatcacacacttcattcgctttgaggccctgctttaaccttaagttatagccctgtccaccgaggaaggtcagggtgagagcctagattcctcctgtgtcaagggtccctcgcattcttttactgtaaacaaacaatgccttaaattgtgtcttgttttctgttcctatgggtgctattcatctggaaggcctgcttccaggcctctttgctgtcagcccttctgagacaggacctggcttcaggactgtggactgggctgctggcctgcttgcttcctcccttccccattcctagcagggcctgaggccctcctcttctcgcccttcccaccatgccagaatgggaagttgtgacgttgcagctccaaccgacgtgctcatagtgatcagctgtgcaggagccatgaggcaccaacctctccccgcagggcaaagcctgtgcccccatcatctcactcctttgcctgcactgccagggtggggcccaccaagattcctgatcatgacgggaagctgagtgaccctgaggccttaagcttccccagtcttggccccaaatgcagtcaccagcaagttttccattttccaagtccaagggcacaattgttgatgaccgtgtgacaatagagcgaagccccggggagtgaacggtccaacctctgcattcagttaggagctcttcacatgaatcacatccttatctgtcaccttgtgtcacattttaaagtgacttttattttgcacaaataatttttattcagaataataaatcactctttatcatagtatcttctcttccctcttcccctttagtttggatagcctaactctgagaagttaacccttaaacagttttctggaagagactgaatttctgggtccttgcagctgtgatggtttcagagctcagactgatcaggcatcaagctaccctcaagagtttctgggctggatgtttcagaacaacatctacaccagtaaagtgtaataggtcagtttcaaaacgaccaaaagacccaccactgtattttgaccaaataatgacaacttctttagaaatttgaatggcttggtgaggaaagtagttgtcaccagggcctcattttgtagttgagccttacaatgcttagtagttcatcttctttttgagcaaagactagaatactttcctcctaagagaaactcccaggtgataaaagttgatgccatcaaaccttgacaccgggtgctctgcacacccacgcggatgttgcacctcattctcccgatgactattcaaatcagcatctagaggctgaatgacaatgccaaacactccacctctgatcagaaccatgcagtgttaacactttaacctacattgaatctgattctacctgttaacttttaaaaagtcgtaagtttggatgaaagtgcaagatgtggaacatcaactacctattttccttgggtttttccactctgcaaactgtcctggtttttcacaccaatgaagtattatagatgccaatccaaaacctcagaatttcaggcaccacaaaaacaggtaattttctatcccttataagtttgtcttttctttcagaaacatctcttagcctaatttgaaatagcacaatcacaattcaaaatgtttagtcttctcactaattgagtctgcttccacgtcctctcccaggaacattcttagctcggactcttgaagaatctctttagattttgttggcaaaagccttatagaagcagtaagaggcttgaccacgccggaagagtcctggagctaaagctggaagacactcagctctctaagcaggggctcggccaaacatgggagttaagtgctgcttgtcttcccagtgttggtttgaaccctgtgagcctgagacagagagggccaggcaccaaccacaaggcgggaaagtccatgggtagaccctccccctggagggaagcatttctagtttttgctccttgactgtccagagtgtacaaatgttcataacgccattgaagggattatttcttgcatgcatatgctgaatttttttaagcaaatggatcatggcaccccaaaatgaaagttatagaaagctgtctacaactgtggagttggtagctggtaacattgttgtctcaagaacaactcacctctctccctaggactaatttttgtctctctcagttgaacatgttttgtcattcaagatcagtcaggtgcattctggcaactgacatacttgatggaggattgattcggtagagagcagtagaaatcttgttctaactgtgcctggtgagagactttggccccctccctccctataaggctgtggaacctgaggaagtagatacttgaagagattctgtttaggaagaaactcactctcttttgccagttgaatttatagagcattttttttcttaccaagatggccagtatcattttacccccacctcccaagccccaagaggtgtaccttttcagatgccattttacaggcggaaatgctccatgaaacaggaagccacttgcaagcaacatctgctctgttcctcaggtggggcccagagcccttccccgagactgctgatgtctgtaaccactggggagcactgccaaaaatacagctttctggtttgtgagcccataaatgacttaaatcagctttacatcatttttacatatcaagtggtttcatgttaaaaaacaaactcctagtcctttagaaataacagattctctgcacaaaaccacccattcattcatttattcattcacagcactagcaagtgctgcctatgctgagaacaagtcagatctgatccctgccctcatggacctgaccactcaacaaacagtccccaccacacctatctccttaggcaagactttgcctctctcctagtcctgagtataaatcctgtgcatagattcctctagaaaggcatcaaaaggctcaacagactgaatggcctcttggtctgcgaaaattcagttgcaatgaggatgaagtcactatcctagaggctgcttggcccagaagagccaggcacagagctgcagttgggcacgccaaggattccaaaggtggaatgagagagtagggtcaaactgtcacagtatctgctccataggtttctgtttttaatttcaatgttaaatacaactacaatatgagcgagaactgcattttcttgggtgttgagaacttgtaccatggacttcagaccgccttgcagccgtatgctgcacaagcgtgtacaccccctgggcagcctcaaaaccccgcttacagcagcaacacaggagatcatctgtccattttagaaccattaatctctttatccattgctgaacgactgtgactattcagtaacgaagtaatagtaattaattagtatggtataatctttaataaatttcgtgccaaaatgcatggttttccacttagcattcaaaatgttgcatagagagtagttttcaatttcttatgtactcttcgaagtaagttgaaaatcagtttctacattttaattcgtttcctgttaaatctgttgcactctcctgggctgtctttttctccagcagacccctgcatgcagttgtgtaaggactttctctaattcttgtgaatcgtctcacccgcagtaaccactgaacgtcaatcagccctccatggggttctttcgatttttggtgaagtattttgttacctcagtcttgtatcaagttgctgtatttttcagcttgttacattgataataattatttcactaattaaatactttaatgtacaaacatctttgtttactttgaaattaaatgtgttttccaatgaa

SEQ ID NO: 68 - protein, 557 aa, Homo sapiens
MYKRNGLMASVLVTSATPQGSSSSDSLEGQSCDYASKSYDAVVFDVLKVTPEEFASQITLMDIPVFKAIQPEELASCGWSKKEKHSLAPNVVAFTRRFNQVSFWVVREILTAQTLKIRAEILSHFVKIAKKLLELNNLHSLMSVVSALQSAPIFRLTKTWALLNRKDKTTFEKLDYLMSKEDNYKRTREYIRSLKMVPSIPYLGIYLLDLIYIDSAYPASGSIMENEQRSNQMNNILRIIADLQVSCSYDHLTTLPHVQKYLKSVRYIEELQKFVEDDNYKLSLRIEPGSSSPRLVSSKEDLAGPSAGSGSARFSRRPTCPDTSVAGSLPTPPVPRHRKSHSLGNNMMCQLSVVESKSATFPSEKARHLLDDSVLESRSPRRGLALTSSSAVTNGLSLGSSESSEFSEEMSSGLESPTGPCICSLGNSAAVPTMEGPLRRKTLLKEGRKPALSSWTRYWVILSGSTLLYYGAKSLRGTDRKHYKSTPGKKVSIVGWMVQLPDDPEHPDIFQLNNPDKGNVYKFQTGSRFHAILWHKHLDDACKSNRPQVPANLMSFE

SEQ ID NO: 69 - DNA, 8598 bp, Homo sapiens
  source 1..8598: mol_type = unassigned DNA
gtacgtccaagatggcggcgccctgtaggctggagggactgtgaggtaaacagctgagggggaggagacggtggtgaccatgaaagacaccaggttgacagcactggaaactgaagtaccagttgtcgctagaacagtttggtagtggccccaatgaagaaccttcagaacctgtagcacacgtcctggagccagcacagcgccttcgagcgagagaatggcccaacaagcaaatgtcggggagcttcttgccatgctggactcccccatgctgggtgtgcgggacgacgtgacagctgtctttaaagagaacctcaattctgaccgtggccctatgcttgtaaacaccttggtggattattacctggaaaccagctctcagccggcattgcacatcctgaccaccttgcaagagccacatgacaagcacctcttggacaggattaacgaatatgtgggcaaagccgccactcgtttatccatcctctcgttactgggtcatgtcataagactgcagccatcttggaagcataagctctctcaagcacctcttttgccttctttactaaaatgtctcaagatggacactgacgtcgttgtcctcacaacaggcgtcttggtgttgataaccatgctaccaatgattccacagtctgggaaacagcatcttcttgatttctttgacatttttggccgtctgtcatcatggtgcctgaagaaaccaggccacgtggcggaagtctatctcgtccatctccatgccagtgtgtacgcactctttcatcgcctttatggaatgtacccttgcaacttcgtctcctttttgcgttctcattacagtatgaaagaaaacctggagacttttgaagaagtggtcaagccaatgatggagcatgtgcgaattcatccggaattagtgactggatccaaggaccatgaactggaccctcgaaggtggaagagattagaaactcatgatgttgtgatcgagtgtgccaaaatctctctggatcccacagaagcctcatatgaagatggctattctgtgtctcaccaaatctcagcccgctttcctcatcgttcagccgatgtcaccaccagcccttatgctgacacacagaatagctatgggtgtgctacttctaccccttactccacgtctcggctgatgttgttaaatatgccagggcagctacctcagactctgagttccccatcgacacggctgataactgaaccaccacaagctactctttggagcccatctatggtttgtggtatgaccactcctccaacttctcctggaaatgtcccacctgatctgtcacacccttacagtaaagtctttggtacaactgcaggtggaaaaggaactcctctgggaaccccagcaacctctcctcctccagccccactctgtcattcggatgactacgtgcacatttcactcccccaggccacagtcacaccccccaggaaggaagagagaatggattctgcaagaccatgtctacacagacaacaccatcttctgaatgacagaggatcagaagagccacctggcagcaaaggttctgtcactctaagtgatcttccagggtttttaggtgatctggcctctgaagaagatagtattgaaaaagataaagaagaagctgcaatatctagagaactttctgagatcaccacagcagaggcagagcctgtggttcctcgaggaggctttgactctcccttttaccgagacagtctcccaggttctcagcggaagacccactcggcagcctccagttctcagggcgccagcgtgaaccctgagcctttacactcctccctggacaagcttgggcctgacacaccaaagcaagcctttactcccatagacctgccctgcggcagtgctgatgaaagccctgcgggagacagggaatgccagacttctttggagaccagtatcttcactcccagtccttgtaaaattccacctccgacgagagtgggctttggaagcgggcagcctcccccgtatgatcatctttttgaggtggcattgccaaagacagcccatcattttgtcatcaggaagactgaggagctgttaaagaaagcaaaaggaaacacagaggaagatggtgtgccctctacctccccaatggaagtgctggacagactgatacagcagggagcagacgcgcacagcaaggagctgaacaagttgcctttacccagcaagtctgtcgactggacccactttggaggctctcctccttcagatgagatccgcaccctccgagaccagttgcttttactgcacaaccagttactctatgagcgttttaagaggcagcagcatgccctccggaacaggcggctcctccgcaaggtgatcaaagcagcagctctggaggaacataatgctgccatgaaagatcagttgaagttacaagagaaggacatccagatgtggaaggttagtctgcagaaagaacaagctagatacaatcagctccaggagcagcgtgacactatggtaaccaagctccacagccagatcagacagctgcagcatgaccgagaggaattctacaaccagagccaggaattacagacgaagctggaggactgcaggaacatgattgcggagctgcggatagaactgaagaaggccaacaacaaggtgtgtcacactgagctgctgctcagtcaggtttcccaaaagctctcaaacagtgagtcggtccagcagcagatggagttcttgaacaggcagctgttggttcttggggaggtcaacgagctctatttggaacaactgcagaacaagcactcagataccacaaaggaagtagaaatgatgaaagccgcctatcggaaagagctagaaaaaaacagaagccatgttctccagcagactcagaggcttgatacctcccaaaaacggattttggaactggaatctcacctggccaagaaagaccaccttcttttggaacagaagaaatatctagaggatgtcaaactccaggcaagaggacagctgcaggccgcagagagcaggtatgaggctcagaaaaggataacccaggtgtttgaattggagatcttagatttatatggcaggttggagaaagatggcctcctgaaaaaacttgaagaagaaaaagcagaagcagctgaagcagcagaagaaaggcttgactgttgtaatgacgggtgctcagattccatggtagggcacaatgaagaggcatctggccacaacggtgagaccaagacccccaggcccagcagcgcccggggcagtagtggaagcagaggtggtggaggcagcagcagcagcagcagcgagctttctaccccagagaaacccccacaccagagggcaggcccattcagcagtcggtgggagacgactatgggagaagcgtctgccagcatccccaccactgtgggctcacttcccagttcaaaaagcttcctgggtatgaaggctcgagagttatttcgtaataagagcgagagccagtgtgatgaggacggcatgaccagtagcctttctgagagcctaaagacagaactgggcaaagacttgggtgtggaagccaagattcccctgaacctagatggccctcacccgtctcccccgaccccggacagtgttggacagctacatatcatggactacaatgagactcatcatgaacacagctaaggaatgatggtcaatcagtgttaacttgcatattgttggcacagaacaggaggtgtgaatgcacgtttcaaagctttcctgtttccagggtctgagtgcaagttcatgtgtggaaatgggacggaggtcctttggacagctgactgaatgcagaacggtttttggatctggcattgaaatgcctcttgaccttcccctccacccgccctaaccccctctcatttacctcgcagtgtgttctaatccaagggccagttggtgttcctcagtagctttactttcttcctttcccccccaaatggttgcgtcctttgaacctgtgcaatatgaggccaaatttaatctttgagtctaacacaccactttctgctttcccgaagttcagataactgggttggctctcaattagaccaggtagtttgttgcattgcaggtaagtctggttttgtcccttccaggaggacatagcctgcaaagctggttgtctttacatgaaagcgtttacatgagactttccgactgcttttttgattctgaagttcagcatctaaagcagcaggtctagaagaacaacggtttattcatacttgcattcttttggcagttctgataagcttcctagaaagttctgtgtaaacagaagcctgtttcagaaatctggagctggcactgtggagaccacacaccctttgggaaagctcttgtctcttcttcccccactacctcttatttatttggtgtttgcttgaatgctggtactattgtgaccacaggctggtgtgtaggtggtaaaacctgttctccataggagggaaggagcagtcactgggagaggttacccgagaagcacttgagcatgaggaactgcacctttaggccatctcagcttgctgggccttttgttaaacccttctgtctactggcctccctttgtgtgcatacgcctcttgttcatgtcagcttatatgtgacactgcagcagaaaggctctgaaggtccaaagagtttctgcaaagtgtatgtgaccatcatttcccaggccattagggttgcctcactgtagcaggttctaggctaccagaagaggggcagctttttcataccaattccaactttcaggggctgactctccagggagctgatgtcatcacactctccatgttagtaatggcagagcagtctaaacagagtccgggagaatgctggcaaaggctggctgtgtatacccactaggctgccccacgtgctcccgagagatgacactagtcagaaaattggcagtggcagagaatccaaactcaacaagtgctcctgaaagaaacgctagaagcctaagaactgtggtctggtgttccagctgaggcagggggatttggtaggaaggagccagtgaacttggctttcctgtttctatctttcattaaaaagaatagaaggattcagtcataaagaggtaaaaaactgtcacggtacgaaatcttagtgcccacggaggcctcgagcagagagaatgaaagtcttttttttttttttttttttttagcatggcaataaatattctagcatccctaactaaaggggactagacagttagagactctgtcaccctagctataccagcagaaaacctgttcaggcaggctttctgggtgtgactgattcccagcctgtggcagggcgtggtcccaactactcagcctagcacaggctggcagttggtactgaattgtcagatgtggagtattagtgacaccacacatttaattcagctttgtccaaaggaaagcttaaaacccaatacagtctagtttcctggttccgttttagaaaaggaaaacgtgaacaaacttagaaagggaaggaaatcccatcagtgaatcctgaaactggttttaagtgctttccttctcctcatgcccaagagatctgtgccatagaacaagataccaggcacttaaagccttttcctgaattggaaaggaaaagaggcccaagtgcaaaagaaaaaacattttagaaacggacagcttataaaaataaagggaagaaaggaggcagcatggagagaggcctgtgctagaagctccatggacgtgtctgcacagggtcctcagctcatccatgcggcctgggtgtccttttactcagctttataacaaatgtggctccaagctcaggtgcctttgagttctaggaggctgtgggttttattcaactacggttgggagaatgagacctggagtcatgttgaaggtgcccaacctaaaaatgtaggctttcatgttgcaaagaactccagagtcagtagttaggtttggtttggttttggacatgataaacctgccaagagtcaacaggtcacttgatcatgctgcagtgggtagttctaaggatggaaaggtgacagtattactctcgagaggcaattcagtcctgggcaaaggtattagtacaataagcgttaagggcagagtctaccttgaaaccaattaagcagcttggtattcataaatattgggattggatggcctccatccagaaatcactatgggtgagcatacctgtctcagctgtttggccaatgtgcataacctactcggatccccacctgacactaaccagagtcagcacaggccccgaggagcccgaagtctgctgctgtgcagcatggaattcctttaaaaaggtgcactacagttttagcggggagggggataggaagacgcagagcaaatgagctccggagtccctgcaggtgaataaacacacagatctgcatctgatagaactttgatggattttcaaaaagccgttgacaaggctctgctatacagtctataaaaattgttattatgggattggaagaaacacgtggtcatgaatagaaaaaaaacaaacccaaaggtaggaaggtcaaggtcatttcttagatggagaagttgtgaaagatgtccttggagatgagttttaggaccagcattactaaggcaggtgggcagacagtgacctctctaggtgtgtccacagagtttttcaggagagaaaactgcctgacctttgggactaagctgcggaatcttcttactaagcttgaagagtggagaggcgagaggtgagctactttgtgagccaaagcttatgtgacatggttggggaaacagtccaaactgttctgagaaggtgaactgttacgacccaggacaattagaaaaattcacccaccatgccgcacattactgggtaaaagcagggcagcagggaacaaaactccagactcttgggccgtccccatttgcaacagcacacatagtttctggtatatttgttgggaaagataaaactctagcagttgttgaggggaggatgtataaaatggtcatggggatgaaaggatctctgagaccacagaggctcagactcactgttaagaatagaaaactgggtatgcgtttcatgtagccagcagaactgaagtgtgctgtgacaagccaatgtgaatttctaccaaatagtagagcataccacttgaagaaggaaagaaccgaagagcaaacaaaagttctgcgtaatgagactcaccttttctcgctgaaagcactaagaggtgggaggaggcctgcacaggctggaggagggtttgggcagagcgaagacccggccaggaccttggtgagatggggtgccgcccacctcctgcggatactcttggagagttgttcccccagggggctctgccccacctggagaaggaagctgcctggtgtggagtgactcaaatcagtatacctatctgctgcaccttcactctccagggtacatgctttaaaaccgacccgcaacaagtattggaaaaatgtatccagtctgaagatgtttgtgtatctgtttacatccagagttctgtgacacatgccccccagattgctgcaaagatcccaaggcattgattgcacttgattaagcttttgtctgtaggtgaaagaacaagtttaggtcgaggactggcccctaggctgctgctgtgacccttgtcccatgtggcttgtttgcctgtccgggactcttcgatgtgcccaggggagcgtgttcctgtctcttccatgccgtcctgcagtccttatctgctcgcctgagggaagagtagctgtagctacaagggaagcctgcctggaagagccgagcacctgtgcccatggcttctggtcatgaaacgagttaatgatggcagaggagcttcctccccacttcgcagcgccacattatccatcctctgagataagtaggctggtttaaccattggaatggacctttcagtggaaaccctgagagtctgagaacccccagaccaacccttccctccctttccccacctcttacagtgtttggacaggagggtatggtgctgctctgtgtagcaagtactttggcttatgaaagaggcagccacgcattttgcactaggaagaatcagtaatcacttttcagaagacttctatggaccacaaatatattacggaggaacagattttgctaagacataatctagttttataactcaatcatgaatgaaccatgtgtggcaaacttgcagtttaaaggggtcccatcagtgaaagaaactgattttttttaacggactgcttttagttaaattgaagaaagtcagctcttgtcaaaaggtctaaactttcccgcctcaatcctaaaagcatgtcaacaatccacatcagatgccataaatatgaactgcaggataaaatggtacaatcttagtgaatgggaattggaatcaaaagagtttgctgtccttcttagaatgttctaaaatgtcaaggcagttgcttgtgtttaactgtgaacaaataaaaatttattgttttgcacta

SEQ ID NO: 70 - protein, 1164 aa, Homo sapiens
MAQQANVGELLAMLDSPMLGVRDDVTAVFKENLNSDRGPMLVNTLVDYYLETSSQPALHILTTLQEPHDKHLLDRINEYVGKAATRLSILSLLGHVIRLQPSWKHKLSQAPLLPSLLKCLKMDTDVVVLTTGVLVLITMLPMIPQSGKQHLLDFFDIFGRLSSWCLKKPGHVAEVYLVHLHASVYALFHRLYGMYPCNFVSFLRSHYSMKENLETFEEVVKPMMEHVRIHPELVTGSKDHELDPRRWKRLETHDVVIECAKISLDPTEASYEDGYSVSHQISARFPHRSADVTTSPYADTQNSYGCATSTPYSTSRLMLLNMPGQLPQTLSSPSTRLITEPPQATLWSPSMVCGMTTPPTSPGNVPPDLSHPYSKVFGTTAGGKGTPLGTPATSPPPAPLCHSDDYVHISLPQATVTPPRKEERMDSARPCLHRQHHLLNDRGSEEPPGSKGSVTLSDLPGFLGDLASEEDSIEKDKEEAAISRELSEITTAEAEPVVPRGGFDSPFYRDSLPGSQRKTHSAASSSQGASVNPEPLHSSLDKLGPDTPKQAFTPIDLPCGSADESPAGDRECQTSLETSIFTPSPCKIPPPTRVGFGSGQPPPYDHLFEVALPKTAHHFVIRKTEELLKKAKGNTEEDGVPSTSPMEVLDRLIQQGADAHSKELNKLPLPSKSVDWTHFGGSPPSDEIRTLRDQLLLLHNQLLYERFKRQQHALRNRRLLRKVIKAAALEEHNAAMKDQLKLQEKDIQMWKVSLQKEQARYNQLQEQRDTMVTKLHSQIRQLQHDREEFYNQSQELQTKLEDCRNMIAELRIELKKANNKVCHTELLLSQVSQKLSNSESVQQQMEFLNRQLLVLGEVNELYLEQLQNKHSDTTKEVEMMKAAYRKELEKNRSHVLQQTQRLDTSQKRILELESHLAKKDHLLLEQKKYLEDVKLQARGQLQAAESRYEAQKRITQVFELEILDLYGRLEKDGLLKKLEEEKAEAAEAAEERLDCCNDGCSDSMVGHNEEASGHNGETKTPRPSSARGSSGSRGGGGSSSSSSELSTPEKPPHQRAGPFSSRWETTMGEASASIPTTVGSLPSSKSFLGMKARELFRNKSESQCDEDGMTSSLSESLKTELGKDLGVEAKIPLNLDGPHPSPPTPDSVGQLHIMDYNETHHEHS

SEQ ID NO: 71 - DNA, 4557 bp, Homo sapiens
  source 1..4557: mol_type = unassigned DNA
agctgccgccggcgtcgcggggctccaggctgcggggcggacgtagcgccgagcgatccaccgcggagccgcgcgaggccgcccgagccccagccccggccccggccccagccccggccccggccccggccccggccccggccccggtcccggccccgagccccgagcgagcgccgccgcctcgcgctgcgggtccccggacgcgccgccaccgccgggaaggcagccggcgccgggcaagttgcgagcgcgcccctccgaccaggaaagtttccccccggccgccggcgggatttggcgcggggtccggcagccgccgctggaagagtgtatccagaagctactgtcaggaggacactgtaagagaaccttggcacctctgggcccaaagggaaagacaccagtggaaagaggtgagcatctcctggaccaccctcaagcttcaggtgagctgagcttctaacactaccatcaaagcaactggaaccccttgaatttgatttctggagacgcgagcataatccttttgcagacatctcaacgctggctctccaggtggagcaccatggaaggcgactgtctgagctgcatgaagtatctgatgtttgtattcaatttcttcatatttctgggcggggcctgcctgctggccatcggcatctgggtcatggtggaccccaccggcttccgggagatcgtggctgccaatcctctgctcctcacgggcgcctacatcctcctggccatggggggcctgctctttctgctcggcttcctgggctgctgcggggccgtccgtgagaacaagtgtctgctgctatttttcttcctgttcatcctgatcatcttcctggcagagctctcagcagccatcctggccttcatcttcagggaaaatctcacccgagaattcttcaccaaggagctcaccaagcactaccagggcaataacgacacagacgtcttctctgccacctggaactcggtcatgatcacatttggttgctgcggggtcaacgggcctgaagactttaagtttgcatctgtgtttcgactcctgaccctggatagtgaagaggtgccggaggcctgctgccggagggaaccccaaagtcgggacggggtcctgctgagccgggaggagtgcctcctgggaaggagcctattcctaaacaagcagggctgttacacggtgatcctcaacaccttcgagacctacgtctacttggccggagcccttgccatcggggtactggccatcgagcttttcgccatgatctttgccatgtgcctcttccggggcatccagtagagggtatggcctgaagcctgaagactcgccccacccaccactgcccagcacccagtgtcctcccgtgcccctccccgctgtcctcttggccccaggggagaagatgaggccatcagagatggccaggagaagggccaggggaatagagctatttttttaacaaaacaaaatgaagacaaaaatatggactgatgtatcctcgcctggactcagggcaggtgccgtgggttctccagagaccccagcaactggcccaggatgcaggctgctctagagaccaaaggaacaccaggcccagcgccctctttggtccagccagaccctgggccctctctcctcactgcaccaggacctgatgccaagaaagtgaggatttaggcaacaaggaggcaaaagcaaatctcagagaagtcatcaaagcccaggggctgcacggagattcagctgccatgtctttgaggactcggaacccacagcgcaatccctggtccactggaggatggactgttccccccttcaggccggggccaagagtgagctgctaacacggcatccaagaatggcccagctggtaggatccttgctttccccatctgttggaccaaataatctcaaaggcccagagaggacagtgaccatccacagacacacagcacaggcgtgacagttctgaaaagaagctgagttgtctccaggctgctgtcactgcatccaggactctgtcagctctgctgcccacatgcacccctggcctcagcatcccggttcccccagacaagagagggcaagtcagccaggaactgcctcctccctgctctacagctaagaaaacaccattccatgacttcccaccgcgccctcgctcctacctccccacacctctctctgagtccccaggaacacacagaggtgcacatcacattcccttgtccacactgcccgcctctcccacatgccacccccttccctgtccttccccaactccccagctccaagagtggaagaaatccccaagatcatctgggtctccctctccacacccagaactgaggcttggatatcttcttcaacatccttgccaagacttctccaccctcttgcatacctccagggacagagagcttactacctcccaaggcagcctcctgcctttggactgctctgactttagcatcagcttaatacacagaagaggtttctgtttttctgtggctctgtccatggcatcccacttgcccactctccttcctggagtgttgaggtcacacattgactcccctgagccctcttctctccaggctaaagaatcccgaaggcatcgaggccatttctgctgcaacaaggtttcctgtctcttcactgtccgcacatttcttagtattccctccaggcttgggcagggagactcgcagatgcacacccacaagtattcagttctcaaactctagacgagtgttggcaactggattgcaagatgctcctaccctgatagatcaggggtggctgctggaggctgtgctggggatctgaggtttggtctgggctcagtgggagacggcagtgcaatcctgatgagtgatgtctgccaggcaccgtaagtttgattagtgatgtctgccacgggcagggatggaaggagcagtgtgatgtctgctctcttctctcccctctgtccctcttcaggaagaaagagctcattctgtctcacaagccaccggcatcctgtatcagcttccagcctcccctcaggctttccagtcaccagggacactcggagccacagcctagagccccgtgttccctggcctgtgcgtctgcccccttctgagatgcagccagaagctctgtgcctgctgcaaagattcaggtggaccctcctctaatctcctcctgctgtgcccgccagtccttgccctcccaccaggtctctgagctcagtgttaccaaattcgccctttaacagcttgctctggcaaccccataaatgacacctgaggtccgtagaagctaagctcctgagacccagggggacctgccactggtaccgcggcccagcctggggcctgggggctgcccctcttgaaccacccacatgcttagccccagctttttggaagaggcaaatggctggtctgaggatgacacacaaaaacaaaaacaaaaaacaaaaaacccatgctgggcaggactgaggcaaattgcacagctttatggctctaatccaggggcatcccaggcttctggggcccacagaagtcagagggaggacccaagagaaagggctggtcatgaagggaaatctggctaaggtgggttcaagggcagacacaagactgcccctcagcagctttctacaaatgtgccaaggaaccctcaatcagccctgattcagctcgccagccagccactggccaccctcttaggctggaaagggaagacaggcagtttctgctcctgttggcattcgctcaggctggtagctatttgcaagactgcctgaggccattccttggaggcaaggcaaagaaagctcagcccaaatcaggcttgagcctccctccagagcacagggagaaacagggtttagctggcttggtccagatacaacccacagcaggttctggtggtggctggggttgtgggggaggggtgggcagggatacctctttgtttcttttcaccccgaaatacaacagcccataacagagacttcctcggaccccactaacagggcaaggaacaagaagactacaccgctcatcacaaaccctgcctgtatcgaaagccactttcctgctctgaagctactgcctcttagagaaagggaatagctctttatgggctgggggtgagggcccctccccagggtccctgttaatttctggccttggtgctcaggcctgtccacagcctcccttgtctatgtctctatccatgcttaaggggcccggacaggatttcccaaaccagccgaggccccagcacccgccgtctccccagaagccccctcctccttcccccatgggtcatatgttgaaagtctattttaaaaactatgttccttgccgtagattgcagagctaatttatcacgtttctctcctgtgagaccccccttttatatgatatatccagaggaagttttgtaatataaaacaggacgcccacactgatggttttgcactggtttttgtgaatgtttcttacaaaaagaaaaaggaacaaagaataaatagtgaccgtgaa

SEQ ID NO: 72 - protein, 248 aa, Homo sapiens
MEGDCLSCMKYLMFVFNFFIFLGGACLLAIGIWVMVDPTGFREIVAANPLLLTGAYILLAMGGLLFLLGFLGCCGAVRENKCLLLFFFLFILIIFLAELSAAILAFIFRENLTREFFTKELTKHYQGNNDTDVFSATWNSVMITFGCCGVNGPEDFKFASVFRLLTLDSEEVPEACCRREPQSRDGVLLSREECLLGRSLFLNKQGCYTVILNTFETYVYLAGALAIGVLAIELFAMIFAMCLFRGIQ

SEQ ID NO: 73 - DNA, 5823 bp, Homo sapiens
  source 1..5823: mol_type = unassigned DNA
agccacctcgtcagagcagcatgtggactggctcgccgggtcccctccgtgctctgtgctgtcgccgccgccgcctctgtcagagcagcagctgtcggcagcaggagccccgcacggggcgcggagcagggacgcgctgccaccgcctccccctgcgtcctgctggccgcgtcttctcgggaggtggtagtcgctgttgccgctgagaaacccgcccgctttccacggctggtcgcctggtgaggagttgagactctgcgcctccgcccggacccacatggcttgttacctggtcatcagttcgagacatctcagcaatgggcactaccggggcattaaaggagtcttcaggggacccctgtgcaagaacggatctccctctccggattttgcagaaaagaagtccacagcaaaggccctggaagatgtaaaggcaaacttttactgtgaattatgtgacaagcagtatcacaaacaccaggagtttgacaatcatattaattcttatgaccatgctcataagcagagactgaaagaattaaagcaacgggaatttgctcgaaatgtagcttctaagtcatggaaagatgagaaaaaacaagaaaaagcacttaaacgacttcatcagctggctgagttaaggcagcaatctgaatgtgtttctggaaatggaccagcatacaaagcccccagggtagccatagaaaagcaactccagcaaggaattttccccattaagaatggcagaaaggtatcatgcatgaagagtgctcttctccttaaaggaaaaaatctccccagaatcatatccgataaacagcggtccaccatgccaaatcgacaccaattacaatcagacaggcgttgtttgtttggaaatcaggtactgcaaacatcttcagatctcagcaatgcaaatcacagaacaggagtatcatttactttttccaaaaaagtgcacctaaaattagaatcttcagcatcagttttcagtgagaacacagaagaaacccatgattgtaacaagtcacccatttataaaacaaaacaaactgcagataagtgcaagtgctgcaggtttgcaaataaagatacacaccttaccaaggaaaaagaggtaaatatctcaccaagccatctggaaagtgttttacacaataccatctccataaactctaaaattttgcaagacaaacacgactctattgatgagacactagaagattcaattggcattcatgcttcattctctaaatctaacattcatctttcagatgtagattttactcctaccagcagagaaaaagaaactagaaatacattgaagaacactttagaaaattgtgttaatcacccatgccaagcaaatgcttccttcagcccaccaaacatttacaaccatagtgatgccaggatatctgaatgcctggatgagttttcatcactggagccaagtgaacaaaagagtacagtgcatctgaatccaaattccagaatagagaacagagaaaaatctttagataaaacagaaagagttagcaaaaatgttcaaagacttgtaaaagaagcatgtacccataatgtggcatctaaaccactaccttttctccacgttcaaagcaaggatggccacaccactcttcaatggcctacggaacttctgctctttacaaaaacagaaccctgtatctcttatggctgcaacccactgtattttgattttaagctttctcggaacacaaaggaagaccacaatctagaggacttaaaaacagaattgggtaagaagcccttggaattgaagactaaaagagagagccaagtctcaggtttaactgaagaccaacaaaaattgatccaagaagattatcaatatccgaaaccaaagacgatgatagctaatccggattgggaaaaattccagaggaaatataatttggactacagtgattctgagccaaataagagtgaatatactttcagtgcaaatgatttggaaatgaaaaatcctaaagtgcctctttacctcaacacatctctaaaggattgtgctggaaagaataatagtagtgagaacaaacttaaggaagcttcaagggcccattggcaaggctgcagaaaggcagttctaaatgatatagatgaggacctatcttttccttcctacatctctaggtttaaaaagcataaattgattccctgcagtcctcatttggaatttgaagatgaaagacaattcaactgcaagtccagtccttgtacagtagggggtcacagtgaccatgggaaagacttcagtgtaattttgaagagtaaccacatcagcatgaccagcaaggtttccggatgtggaaaccaaagatacaagagatactctccacagtcatgtttgagtagatattcttcctctttggacacatcccctagcagcatgtctagcttgagaagtacttgttcaagtcatagattcaatggtaatagcagaggtaatttgctctgcttccataaaagagaacaccactcagttgaaaggcacaaacggaaatgtctaaagcacaactgcttctacttgtctgatgatataacaaagagcagccaaatgcagtctgaaccacagaaagagaggaactgcaaattgtgggaatcatttaaaaatgaaaaatactcaaaacgtagatattgtcactgcagagaaagacaaaaactgggcaaaaatcaacaacaattttcagggctaaaatctacgagaatcatctattgtgattctaactcacagatttcctgtactggaagcagtaaaaaaccacctaattgccagggaactcagcacgacagattggactcttactcaatagagaaaatgtattacttgaataaaagcaagagaaatcaagagtctttgggcagccctcacatttgtgatctgggaaaagtcaggcccatgaagtgtaactccgggaatatcagctgccttctaaagaactgttccagtggcccttcagaaaccacagaatcaaacactgcagaaggagagaggacccctctaacagcaaaaatccttttagaaagagtacaagccaagaaatgtcaagaacaatcaagtaatgttgagatctcttcaaacagttgtaaaagtgaattagaggctccttcgcaagtcccatgcacaattcaacttgcaccatcaggctgtaacagacaagcattgcctttgtctgaaaaaatacagtatgcaagtgagagcagaaatgatcaagacagtgcaattccaaggactacggagaaagacaaaagcaaaagttcacacacaaataattttacaattttagcagacactgattgtgataaccatctttctaaaggtataattcacctagtaacagagtctcagtcactaaacataaaaagggatgcaacaacaaaagaacaatcaaaacctttaattagtgaaatccaaccttttattcaaagctgtgacccagtaccaaatgaattccctggtgcttttccgtctaataaatatactggtgtgactgattcaacagagacccaagaagaccaaataaatctagacttacaggatgtaagcatgcatataaatcatgtagagggaaatataaactcttactatgacagaactatgcagaaacctgacaaagtcgaagacggattagaaatgtgtcataaatctatctctccccctttaattcaacagcccataacattttctcctgacgaaatagataaatataagatcctacagctacaagcccagcagcatatgcagaagcaactcctatcaaagcatcttcgagttttgcctgctgcagggcctactgccttctctccggcctcaaccgtacagacagttccagttcaccagcacacttctatcaccaccatccaccacacgttcctgcagcattttgctgtttctgcttccttaagttctcatagcagtcacctccctattgctcatctacatcctctttcacaggcacatttcagtcctatttcattttcgactctgactccaaccattatccctgcacaccccactttcttagcaggtcatcccctgcatttagtagctgctacccccttccacccatctcacataacacttcagcctctgccccctacagcatttattcctacattgtttggtcctcacttaaatccagccacaacttctatcatccacttgaatcctttaatccaaccagtattccaaggtcaagatttttgccatcattcttgctctagccagatgcaacagctaaatgaagtgaaagaggccttaaatgtgtccacacacttgaactaataagtgttaaagcccctcctgtggataatttttttaattgtcactacctataaaatcatacatttaaagaagtctgtcaattataagatttaaaatattgctgccaattcaaaatgtgacaaatatataaatatgatctgaattgctaatactaaaacaagagcattttaataattttttagcttgccaaaataataggcaaatttgaataattttatgaaagcgtagagggaagagggaaagagttaaagatttgttttggatgggttctcgcataatgttataaaatagcaacagaaaacattcaagcagagcattaaaattaaaaagaaaaatcaaatgaaatatggcaccctgcgttctaagtatttgttgtgtgaatgaatgagtgttgatttggcttatttgttagataataaaaggtcagtgaaatgaagcaattatttaaagtaatttatgtcgttaactattgactaagttttcaattaatgtagtctgttactgtattttcataggaaataagaacaagacattttctgattaatttaggttgaaaattacattttaatgaaaaacaatctttatagattatacttttgagtttctgtgaaaaataaattatcttcatttcacacaaatctatgtcaagataattttgtaattgtaagaatcagcaaaatctaaaactaagaatgtttaacttttgtataaaaacatatacaaactatcattagtatttgataatgcaaactcaaaaataaaacgtgctcagaataaccttttaaatatattggtcaatctgtcaaatataacaggaactaaaatattgtttattattcttctactcaaaaattttactacgttcagccaatacatgtataaggatagaggtaaaggtttgaaaatgtcagtgtttttacatgtttattgaggatatcttattaaatatctggtgtgttttattcaattgtactagatatttatgaaacaatgaataatgatgaataaattcaccatccatgtaatgttgcaaataatttcaaacattcattcaaaatggtttttggccgccaaaatttgcatttcatacatctatattggtgcataatcctacagttaggtttattattttatgttcttaagcatgagtttcagagaaaatcaaacttaacagaaatggaggtaatcatttatctaatgctgtctatacgaagatgcaaactcctctaccactagaagattagaggatgtcttcttcagccttggctgcaaaaggaagatagctatatttaaggcttgttgtgattcaggtacagttaccctatccacttgtttgcattatcttctttaattttcacaactgtattttgaaatcagtattatcattcctcattacagataaggaaaatgaaatagaaaggttaaatactaaatacttggtaaacagtaggaaaggacatcgttgattcaggagagactggtgagagagaaagaga

SEQ ID NO: 74 - protein, 1349 aa, Homo sapiens
MACYLVISSRHLSNGHYRGIKGVFRGPLCKNGSPSPDFAEKKSTAKALEDVKANFYCELCDKQYHKHQEFDNHINSYDHAHKQRLKELKQREFARNVASKSWKDEKKQEKALKRLHQLAELRQQSECVSGNGPAYKAPRVAIEKQLQQGIFPIKNGRKVSCMKSALLLKGKNLPRIISDKQRSTMPNRHQLQSDRRCLFGNQVLQTSSDLSNANHRTGVSFTFSKKVHLKLESSASVFSENTEETHDCNKSPIYKTKQTADKCKCCRFANKDTHLTKEKEVNISPSHLESVLHNTISINSKILQDKHDSIDETLEDSIGIHASFSKSNIHLSDVDFTPTSREKETRNTLKNTLENCVNHPCQANASFSPPNIYNHSDARISECLDEFSSLEPSEQKSTVHLNPNSRIENREKSLDKTERVSKNVQRLVKEACTHNVASKPLPFLHVQSKDGHTTLQWPTELLLFTKTEPCISYGCNPLYFDFKLSRNTKEDHNLEDLKTELGKKPLELKTKRESQVSGLTEDQQKLIQEDYQYPKPKTMIANPDWEKFQRKYNLDYSDSEPNKSEYTFSANDLEMKNPKVPLYLNTSLKDCAGKNNSSENKLKEASRAHWQGCRKAVLNDIDEDLSFPSYISRFKKHKLIPCSPHLEFEDERQFNCKSSPCTVGGHSDHGKDFSVILKSNHISMTSKVSGCGNQRYKRYSPQSCLSRYSSSLDTSPSSMSSLRSTCSSHRFNGNSRGNLLCFHKREHHSVERHKRKCLKHNCFYLSDDITKSSQMQSEPQKERNCKLWESFKNEKYSKRRYCHCRERQKLGKNQQQFSGLKSTRIIYCDSNSQISCTGSSKKPPNCQGTQHDRLDSYSIEKMYYLNKSKRNQESLGSPHICDLGKVRPMKCNSGNISCLLKNCSSGPSETTESNTAEGERTPLTAKILLERVQAKKCQEQSSNVEISSNSCKSELEAPSQVPCTIQLAPSGCNRQALPLSEKIQYASESRNDQDSAIPRTTEKDKSKSSHTNNFTILADTDCDNHLSKGIIHLVTESQSLNIKRDATTKEQSKPLISEIQPFIQSCDPVPNEFPGAFPSNKYTGVTDSTETQEDQINLDLQDVSMHINHVEGNINSYYDRTMQKPDKVEDGLEMCHKSISPPLIQQPITFSPDEIDKYKILQLQAQQHMQKQLLSKHLRVLPAAGPTAFSPASTVQTVPVHQHTSITTIHHTFLQHFAVSASLSSHSSHLPIAHLHPLSQAHFSPISFSTLTPTIIPAHPTFLAGHPLHLVAATPFHPSHITLQPLPPTAFIPTLFGPHLNPATTSIIHLNPLIQPVFQGQDFCHHSCSSQMQQLNEVKEALNVSTHLN

SEQ ID NO: 75 - DNA, 2012 bp, Homo sapiens
  source 1..2012: mol_type = unassigned DNA
attggcgccaagatggcgatggagatgaggcttccagtggctcgcaagcctcttagcgagagactgggccgcgacactaagaaacatctagtggtgccgggggatacaatcactacggacacaggattcatgcggggccatggaacgtatatgggagaagagaagctcattgcatctgttgctggctctgtggagagagtaaacaagttgatctgtgtgaaagctttgaaaaccagatacattggtgaagtaggagacatcgtagtgggacgaatcacagaggttcaacagaagaggtggaaggtggagaccaactccaggctggattcggtcttgctgctctcgtccatgaaccttcctggaggagagctgaggagaagatctgcagaagatgagcttgcaatgagaggtttcttacaggaaggggaccttatcagtgctgaggtccaggcagtgttctctgacggagctgtctctttgcacacgaggagcctgaaatatggaaaactaggtcagggggttttggtccaggtttccccctccctggtgaaacggcagaagacccactttcatgatttgccatgtggtgcctcagtgattctcggtaacaacggcttcatctggatttacccaacacctgagcacaaagaagaggaagcagggggcttcattgcaaacctggagcctgtctctcttgctgatcgagaggtgatatcccggcttcggaactgcatcatctcgctggtaactcagaggatgatgctgtatgataccagcatcctgtactgctatgaagcatcccttccacatcagatcaaagacatcttaaagccagaaataatggaggagattgtgatggaaacacgccagaggcttttggaacaggagggataaggaggtgctccagaagcacgggactgtggaccttgcaggagtgaagactgtgatgtgtggtccccatatgtggctcagcaaagactcgagagatcatccctttgtctgcattgacggccctgtgacggcctccagcccacaggcctgctttctcctgtcctaacaccaagcctgggtggcagatgaacagtgcttccttgggttgccagctgagtcccggtattagggaatagtttcagctctttcaaagtgcacagtgttacagtcgaatgggctcccatcctggaataatatggagaatcctttgtcttccactcactgtcattcacaaggcacagtgccccatgaaattgccccaatagaaaacatggcatccctgacctccaaatggtctgttttggcctccattcctatatcctttaaatgactgagaatgcagctggtaaagttggaagaataaagttaaccaagcaggccaggcacggtggctcacgcctataatcccagcacttagggaggccaaggcgggcagatcacctgaggtcaatagttcgagaccagcctggccaacaaggtgaaaccccatctctactaaaaatacaaaaaattagccaggtgtggtggtgtgcacctgtagtcccagctatttgggaggctgaggtgggagaatcacttgaacctgggaggcggaggttgcagtgagctgaaatcgcgtaccacactccagcctgggtgacagagcgagactgtgtttcaaagaaaaaaaaaaacaagcagccttttgcttggttggaatctgattttctgttgcgtgttccttgtagccatagatgtgattatgtccacaccgggctgccttaatctgtcctgcttggagagtgacttgtaagatgctgaattattcatgataatacagtgaatgttctgggtccatgtactcagataaacatgaagaaaataagcaaagaaaaatggagactgggaaagcaaagctgttttcatcctataattgaagtagtgtggagcattaacttgtggatgattcagagttaaaagataaaaagacgccagagttctcgctgaagaatgtgagaattcctgtgcattgttttttctga

SEQ ID NO: 76 - protein, 293 aa, Homo sapiens
MAMEMRLPVARKPLSERLGRDTKKHLVVPGDTITTDTGFMRGHGTYMGEEKLIASVAGSVERVNKLICVKALKTRYIGEVGDIVVGRITEVQQKRWKVETNSRLDSVLLLSSMNLPGGELRRRSAEDELAMRGFLQEGDLISAEVQAVFSDGAVSLHTRSLKYGKLGQGVLVQVSPSLVKRQKTHFHDLPCGASVILGNNGFIWIYPTPEHKEEEAGGFIANLEPVSLADREVISRLRNCIISLVTQRMMLYDTSILYCYEASLPHQIKDILKPEIMEEIVMETRQRLLEQEG

SEQ ID NO: 77 - DNA, 6479 bp, Homo sapiens
  source 1..6479: mol_type = unassigned DNA
gcgcagtcgttcgggtgagcgaagatggcggccgagagggaacctcctccgctgggggacgggaagcccaccgactttgaggatctggaggacggagaggacctgttcaccagcactgtctccaccctagagtcaagtccatcatctccagaaccagctagtcttcctgcagaagatattagtgcaaactccaatggcccaaaacccacagaagttgtattagatgatgacagagaagatctttttgcagaagccacagaagaagtttctttggacagccctgaaagggaacctatcctatcctcggaaccttctcctgcagtcacacctgtcactcctactacactcattgctcctagaattgaatcaaagagtatgtctgctcccgtgatctttgatagatccagggaagagattgaagaagaagcaaatggagacatttttgacatagaaattggtgtatcagatccagaaaaagttggtgatggcatgaatgcctatatggcatatagagtaacaacaaagacatctctttccatgttcagtaagagtgaattttcagtgaaaagaagattcagcgactttcttggtttgcacagcaaattagcaagcaaatatttacatgttggttatattgtgccaccagctccagaaaagagtatagtagggatgaccaaggtcaaagtgggtaaagaagactcatcatccactgagtttgtagaaaaacggagagcagctcttgaaaggtatcttcaaagaacagtaaaacatccaactttactacaggatcctgatttaaggcagttcttggaaagttcagagctgcctagagcagttaatacacaggctctgagtggagcaggaatattgaggatggtgaacaaggctgccgacgctgtcaacaaaatgacaatcaagatgaatgaatcggatgcatggtttgaagaaaagcagcagcaatttgagaatctggatcagcaacttaggaaacttcatgtcagtgttgaagccttggtctgtcatagaaaagaactttcagccaacacagctgcctttgctaaaagtgctgccatgttaggtaattctgaggatcatactgctttatctagagctttgtctcagcttgcagaggttgaggagaagatagaccagttacatcaagaacaagcttttgctgacttttatatgttttcagaactacttagtgactacattcgtcttattgctgcagtgaaaggtgtgtttgaccatcgaatgaagtgctggcagaaatgggaagatgctcaaattactttgctcaaaaaacgtgaagctgaagcaaaaatgatggttgctaacaaaccagataaaatacagcaagctaaaaatgaaataagagagtgggaggcgaaagtgcaacaaggggaaagagattttgaacagatatctaaaacgattcgaaaagaagtgggaagatttgagaaagaacgagtgaaggattttaaaaccgttatcatcaagtacttagaatcactagttcaaacacaacaacagctgataaaatactgggaagcattcctacctgaagccaaagccattgcctagcaataagattgttgccgttaagaagaccttggatgttgttccagttatgctggattccacagtgaaatcatttaaaaccatctaaataaaccactatatattttatgaattacatgtggttttatatacacacacacacacacacacacacacacacacacactctgacattttattacaagctgcatgtcctgaccctctttgaattaagtggactgtggcatgacattctgcaatactttgctgaattgaacactattgtgtcttaaatacttgcactaaatagtgcactgcaagaccagaaaattttacaatattttttctttacaatatgttctgtagtatgtttaccctctttatgaagtgaattaccaatgctttgaataatgttcacttatacattcctgtacagaaattacgattttgtgattacagtaataaaatgatattccttgtgaaatattagtaacaaattatttgggtatttaaacattaggaatcttttcaaagcatttttaataggaagaaatttttcttctagatgtatataggtgatgccatgattccttaattattttaattgacaaagctgaactcattaaatgactaaatgaagcttaagttttcttatgaaaccatcataaatcaaagaacatttgactcttgattgtcttgggtaaagaagagaataattgtctttgcttgtttttaaccatctctattacaagtgcccaatttaagaattaggaaaaaaatgtatttatatacttttacaagtcccatggattttgttcatcttctcgttatcttcagaatctcgtactttgcatatatttaatttattcttgtcttttggttttttttcttttaaactaaaagtatatcatctgtgctcacagttgacaaggaatggtaccatgttaaaatataccatgatttgatgaataagcaagcacaacacaaagcaaatttgaagtaaactgaaatttagtttgccttgggcaatgtttttacccattctgtatgctacttagacagctgttatctaatccttaatgctgatgaaaatttaagtcatggctgggtgtggtggctcacgcctgtaatcctagcactttgggaggctgaggtgggtggatcacctgaggtcaggagttcaagactagcctggccaacatggtaaaactccatctctactaaaaatacaaaaattcgcctggtgtggtggggtgtgcttgtggtcccagctacttgggaggctgaggcacaagaatcgcttgaaccaggaaggtggaggttgcagtgagccaagatcatgccgttgcacgccagcctaggcaacaaaagcaaaactccatctcaaaaaaaaaaaaaaaaaaaaaaaagatgactggtgtcagagctatttttgttttttaaaaaatattcttacaaacacccctcttcttgagcttaaccaccacatatcccttccataggaacaagatatggtatgtgtaactaagtcatctctagtgagtcactggatacaatttatatatcgaagtatccttagcatgtgtttgcttctgctttagttgaaatgttaagccttctataggtgttattcatatttatactcccacagcccagaagagtggctgatacataataggttaataataagggcttgatgggccaggcacagtgactcacatgcctgtagtcccagcttcttgggaggctgaggcaggaggatctcttgagcccaggagtttgaggctatagtacactatgatcgtgcctgtgaatacccactaacctacagccagcctggacaacatagtgagaccctgtctctaaaaaaaatgaagacttgatgagttgaacagggaaaacaaaagtttgctcttctaaaaagaaactgaagcagaaatagtacaatcatcttttgttgcttctgggaatacttttcagaaacgaataattcatagtaatatatatcagatgctatcttgttgctccattaatattgagcaaaagataggcttaccaagaatagtttcactataatgatgtcctagtgttatatccacaggtatattacttaacaaaggtatagtttacacataggatggttattcaagttattattaaaatcttccattcagatgtggtacattagaatattcagtacctccaagtaaaataataaatattttcagccttgattaaactctagactcccttccagacttccatttcccttgcccacatttactaaaagatgagtgtgctggaaatttcaagtattatatgagcctctgatacctaccagtatttaaacaggatttaattatttcaacacttcacccatcaatcttcacttctcctgttcagataaaaatgctttcatcccaaataagtaaaacaagaacattaatagtgctaataatataatcttttcattggataggtctgagtttagcgtttgtttgttttcttaatgagatcaaaagttaaagaccaaggcagcaaaatattgtgaaacaagctgaacttgggatcagcatacatttgttcagtcttgtttcatttagtatgactttgggcatatcacttaaatctcttatctataaaacaagtacataaaagtcttgattcctgaagtgcctttctactttaaaatattttgtttaaaatatcaaacttcaatataaatatgttggaaaacaattctaatcaaagaacttctattaagtaaaacaagacaaaatgtgtgtaaaaatatgcctaacacatagatttccttgattgtattttctactgaactattttcaagtattcagctttatactaatatattaatctcaaaatacctttgtttcattgttgctactccttaaacttttttctttattatctagatctagtaaagttttctgcattcattgtattaatgttggttcaaaagtaattgcgtcctttgtcgttatataaattgttttataaatggctttaactatgacataagccttttaacataatcagctttctttaaaggaggaagtcagatgagtagagaaggaaagttatattcctttacaaatgctcttaattttaatgtcttaagggaattatagagacttccattattctgccttaaataagggatgttacttcagttttcttgtcatggctccctgcatgggcagtcatgttaacaagagtcagggagatagcagatattccataatagtgactgcagataggccagtcaaagtcattttgttttaaaggaagggaggttgctggtagttaggtgggaaaataacacctgtgaaaaggaagtccaagtcactggctcagggactttggaggcattttcatatcatattcactagtcaagaaacgtgtagattatatgtgtgagtgacatagtctgtttcaaagtattagaaaagagacaatagaatcacccaccctcccttcgacacacacttccttatgtaaaatcagaatcaaataattatcattcctaaccctcacagtgaaggatttggtttcatgtaacaatgaaagtagataatgggtcagacatggaaatagcaataaaagggaattacaacaagataaatgtgagtaatagttcattttcctgcatttttgatgagggccatgaagataggtcatggtaatctatggagaaggtccttaagagcatagaacaatataaatataatatgggccatatatataattttaaaattttctacagccaaatgttaaaaagtaaaaaacaattttaataatccagtagctccaaaatattattttaatatgtaatccaatatattgaagttataagatattttacattctttttttgatatctgatgtatattttacactttcatcacatctccattcagaccggctacaatttgatcagttgtcacatgtggatgactagtggctatgataatggacagcataggtttagacaatgcctattcaactattcaacttgttttacatttataaatctcaaaatgcctttatttcattgttggtactcttttttattacccagatacaataaagttttccaagtttcattgtacacatgaaatgagtttactgtggaaatgaaaatgcagagtaagtccaacagctagtttatcatcctgaacccagtgaagaaatcaaagctgaataaaagcgactcctagttgttactctcaagggcaggtataatttatgtgcaagactttgtagcataatgttttgcaagtagtggatgttcaatatctacaataaatgaattgtagtactatattgcttgaaggaagtttataacctgtctagaacaactttctcaattttacaactaaactaggagatccagggcttaaatcatgattgaactgattattcctgttaaatttatgtgtgaaccatatgtgaacattctttgaaaatgtatgataagcatttcaagatttctgttttaattattaacatttagaattctaggaaatcttttgggatacagagaacttatttcttccaactgttgcttcagacgctgataagattgtagaagatagggaagctgtagtttactatctagttttttaatgcacatgaagtaatttaaaatgctcttcttcctttcttgtcacatacttttaaaaaattctctaaagattgaataattttaagtatttcttaactgggcaaaatggaaactttgtaactgaagccagtcaaggtgaatacacagaaaatcattttcttattaaggtcagttaatgggataaaccgttttaagagagttgagaaaaaataaagttctattttaaatttatacatgtaactttaataaaaactaagtcagacaaaatttaacaaaa

SEQ ID NO: 78 - protein, 519 aa, Homo sapiens
MAAEREPPPLGDGKPTDFEDLEDGEDLFTSTVSTLESSPSSPEPASLPAEDISANSNGPKPTEVVLDDDREDLFAEATEEVSLDSPEREPILSSEPSPAVTPVTPTTLIAPRIESKSMSAPVIFDRSREEIEEEANGDIFDIEIGVSDPEKVGDGMNAYMAYRVTTKTSLSMFSKSEFSVKRRFSDFLGLHSKLASKYLHVGYIVPPAPEKSIVGMTKVKVGKEDSSSTEFVEKRRAALERYLQRTVKHPTLLQDPDLRQFLESSELPRAVNTQALSGAGILRMVNKAADAVNKMTIKMNESDAWFEEKQQQFENLDQQLRKLHVSVEALVCHRKELSANTAAFAKSAAMLGNSEDHTALSRALSQLAEVEEKIDQLHQEQAFADFYMFSELLSDYIRLIAAVKGVFDHRMKCWQKWEDAQITLLKKREAEAKMMVANKPDKIQQAKNEIREWEAKVQQGERDFEQISKTIRKEVGRFEKERVKDFKTVIIKYLESLVQTQQQLIKYWEAFLPEAKAIA

SEQ ID NO: 79 - DNA, 6719 bp, Homo sapiens
  source 1..6719: mol_type = unassigned DNA
aatccatcatggcggccgcggggtttggtgtctgtgcctgagcagcgctggagccggagccggttcccgggtcctgcggctgaggagcccctccgctgtccacggcccctaccggcggggggcggctgggtccctcggcggagctcgggagatgtgactgcctgagggcggtggtggtgtcagcgtccggggccgggggagggggtgtctcgggcagagacccccgggcttggggcagctgaggcggccgggcctcctctacacggggcccgccttccgctgtctgggccgcgagagtccttcgtcccttacagccccgccccggctttgggacactgcgggtggtctgtttcccccagcttgggacaccccgttttctgaggcgtggaagagcgtcgccccggagtaagctgcccgtgccgcgccccgacagcttccctcagccccaagccgccccttattccggatcccggccccaactttggccacggagcctcccattcaaatccctcccttgctgtcaaggggtctccccttcccccaaggtggctcccgcgagcctctaatgccctgacttcttccaatgtcacctacggcccccttagtctcagctcagccaaaaactttaatgcaaaggaaaagtctggattggttccacaggccttttaaaaagcggacttaaaagttgctggcaatgcattccttttcgtcagagtcgagggcaaactcgctgaaatctgggtgacccgtgtccttttccggagagcaaagcagagaagcgagagcggccactagttcggcaggaaatttgttggaagatgaagaagctaagatagggggttggtgacttccacaggaaaagttctggaggagtagccaaagaccatcagcgtttcctttatgtgtgagaattgaaatgactagcattattgacccttttcagcatcccctgtgaatatttctgtttaggtttttcttcttgaaaagaaattgttattcagcccgtttaaaacaaatcaagaaacttttgggtaacattgcaattacatgaaattgataaccgcgaaaataattggaactcctgcttgcaagtgtcaacctaaaaaaagtgcttccttttgttatggaagatgtctttctgtgattgacttcaattgctgacttgtggagatgcagcgaatgtgaaatcccacgtatatgccatttccctctacgctcgctgaccgttctggaagatcttgaaccctcttctggaaaggggtacctattattactttatggggcagcagcctggaaaagtacttggggaccaaagaaggccaagcttgcctgccctgcattttatcaaaggagcagggaagaaggaatcatcgaggcatgggggtccacactgcaatgtttttgtggaacatgaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccagcggccactcaggacttcagcaagctcctcagttcggtgaaggaaatcagtgacatagtgcagaggtagcagcagtcaggggtcaggtgtcaggcccgtcggagctgcctgcagcacatgcgggctcgcccatacccgtgacagtggctgacaagggactagtgagtcagcaccttggcccaggagctctgcgccaggcagagctgagggccctgtggagtccagctctactacctacgtttgcaccgcctgccctcccgcaccttcctcctccccgctccgtctctgtcctcgaattttatctgtggagttcctgctccgtggactgcagtcggcatgccaggacccgccagccccgctcccacctagtgccccagactgagctctccaggccaggtgggaacggctgatgtggactgtctttttcatttttttctctctggagcccctcctcccccggctgggcctccttcttccacttctccaagaatggaagcctgaactgaggccttgtgtgtcaggccctctgcctgcactccctggccttgcccgtcgtgtgctgaagacatgtttcaagaaccgcatttcgggaagggcatgcacgggcatgcacacggctggtcactctgccctctgctgctgcccggggtggggtgcactcgccatttcctcacgtgcaggacagctcttgatttgggtggaaaacagggtgctaaagccaaccagcctttgggtcctgggcaggtgggagctgaaaaggatcgaggcatggggcatgtcctttccatctgtccacatccccagagcccagctcttgctctcttgtgacgtgcactgtgaatcctggcaagaaagcttgagtctcaagggtggcaggtcactgtcactgccgacatccctcccccagcagaatggaggcaggggacaagggaggcagtggctagtggggtgaacagctggtgccaaatagccccagactgggcccaggcaggtctgcaagggcccagagtgaaccgtcctttcacacatctgggtgccctgaaagggcccttcccctcccccactcctctaagacaaagtagattcttacaaggccctttcctttggaacaagacagccttcacttttctgagttcttgaagcatttcaaagccctgcctctgtgtagccgccctgagagagaatagagctgccactgggcacctgcgcacaggtgggaggaaagggcctggccagtcctggtcctggctgcactcttgaactgggcgaatgtcttatttaattaccgtgagtgacatagcctcatgttctgtgggggtcatcagggagggttaggaaaaccacaaacggagcccctgaaagcctcacgtatttcacagagcacgcctgccatcttctccccgaggctgccccaggccggagcccagatacgggggctgtgactctgggcagggacccggggtctcctggaccttgacagagcagctaactccgagagcagtgggcaggtggccgcccctgaggcttcacgccgggagaagccaccttcccaccccttcataccgcctcgtgccagcagcctcgcacaggccctagctttacgctcatcacctaaacttgtactttatttttctgatagaaatggtttcctctggatcgttttatgcggttcttacagcacatcacctctttgcccccgacggctgtgacgcagccggagggaggcactagtcaccgacagcggccttgaagacagagcaaagcgcccacccaggtcccccgactgcctgtctccatgaggtactggtcccttccttttgttaacgtgatgtgccactatattttacacgtatctcttggtatgcatcttttatagacgctcttttctaagtggcgtgtgcatagcgtcctgccctgccccctcgggggcctgtggtggctccccctctgcttctcggggtccagtgcattttgtttctgtatatgattctctgtggttttttttgaatccaaatctgtcctctgtagtattttttaaataaatcagtgtttacattagaa

SEQ ID NO: 80 - protein, 1149 aa, Homo sapiens
MGQQPGKVLGDQRRPSLPALHFIKGAGKKESSRHGGPHCNVFVEHEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

(19) United States
(12) Patent Application Publication
(10) Pub. No.: US 2025/0149116 A1
(43) Pub. Date: May 8, 2025

(54) ABL1 FUSIONS AND USES THEREOF

(71) Applicant: Foundation Medicine, Inc., Boston, MA (US)

(72) Inventors: Lyle Villamater LOPEZ, Foxborough, MA (US); Christine Anna PARACHONIAK, Boulder, CO (US); Rachel ERBACH, Arlington, MA (US)

(73) Assignee: Foundation Medicine, Inc., Boston, MA (US)

(21) Appl. No.: 18/952,708

(22) Filed: Nov. 19, 2024

Related U.S. Application Data
(63) Continuation of application No. PCT/US2023/067311, filed on May 22, 2023.
(60) Provisional application No. 63/344,989, filed on May 23, 2022.

(51) Int. Cl. G16B 30/00

(52) U.S. Cl. CPC G16B 30/00

(57) ABSTRACT

Provided herein are ABL1 fusion nucleic acid molecules and polypeptides, methods related to detecting ABL1 fusion nucleic acid molecules and polypeptides in cancer, as well as methods of treatment and uses related thereto. Detection of an ABL1 fusion nucleic acid molecule or polypeptide can be used to identify individuals that may benefit from treatment with an anti-cancer therapy.

### CROSS-REFERENCE TO RELATED APPLICATIONS

This application is a continuation application of International Application No. PCT/US2023/067311, filed on May 22, 2023, which claims the benefit of U.S. Provisional Application No. 63/344,989, filed May 23, 2022, the disclosures of each of which are hereby incorporated by reference in their entirety.

### REFERENCE TO AN ELECTRONIC SEQUENCE LISTING

The contents of the electronic sequence listing (197102009301SEQLIST.xml; Size: 350,745 bytes; and Date of Creation: Nov. 13, 2024) are incorporated herein by reference in their entirety.

### TECHNICAL FIELD

Provided herein are Abelson tyrosine-protein kinase 1 (ABL1) fusion nucleic acid molecules and polypeptides, methods related to detecting such ABL1 fusion nucleic acid molecules and polypeptides, as well as methods of diagnosis/treatment and uses related thereto.

### BACKGROUND

Kinases activated by gene fusions are established oncogenic drivers and therapeutic targets, and have been associated with both hematopoietic malignancies and solid tumors. Kinase gene fusions have also been observed in patients following initial treatment with targeted therapies, suggesting that kinase fusions may be an acquired resistance mechanism, and that patients with such fusions could benefit from strategies that target the acquired kinase fusion. See, e.g, Xu et al., Cancer Manag Res (2019) 11:6343-51; Piotrowska et al., Cancer Discov (2018) 8(12):1529-39; Schrock et al., J Thorac Oncol (2018) 13(9):1312-23; and Schrock et al., J Thorac Oncol 2019; 14(2):255-64).

ABL1 encodes the Abelson protein tyrosine kinase, which is involved in cell growth and survival (Colicelli (2010). Science signaling, 3(139), re6). Activating alterations in the ABL1 kinase have been reported in leukemia, including the BCR-ABL1 translocation carried on the Philadelphia chromosome in chronic myelogenous leukemia (CML) (Konopka et al., (1985) PNAS, 82(6), 1810-1814; Chissoe et al., (1995). Genomics, 27(1), 67-82). The BCR-ABL fusion is the best-studied ABL1 alteration and results in an activated ABL1 kinase. Tyrosine kinase inhibitors (TKIs) such as imatinib, nilotinib, dasatinib, ponatinib, bosutinib, and asciminib have been approved to treat certain hematological malignancies with BCR-ABL1 fusions. Treatment with these therapies has been correlated with increased responses for patients with ALL or CML compared with treatment regimens lacking TKIs (Fielding (2011). Hematology. American Society of Hematology. Education Program, 2011, 231-237; Santos et al. (2011). Current hematologic malignancy reports, 6(2), 96-103; Reddy et al. (2012). Genes & cancer, 3(5-6), 447-454; Soverini et al. (2011). Blood, 118(5), 1208-1215; Keller-V Amsberg et al. (2012). Expert review of anticancer therapy, 12(9), 1121-1127).

Thus, there is a need in the art for characterizing the cancer landscape of ABL1 fusions, and for developing methods, compositions, and assays for evaluating and treating patients with such fusions.

All references cited herein, including patents, patent applications and publications, are hereby incorporated by reference in their entirety. To the extent that any reference incorporated by reference conflicts with the instant disclosure, the instant disclosure shall control.

### SUMMARY OF THE INVENTION

In some aspects, provided herein is a method for selecting a therapy for an individual having a cancer or for identifying an individual having a cancer who may benefit from a treatment comprising an Abelson tyrosine-protein kinase 1 (ABL1)-targeted therapy, the method comprising detecting in a sample from the individual an ABL1 fusion nucleic acid molecule, or an ABL1 fusion polypeptide encoded by the ABL1 fusion nucleic acid molecule, wherein the ABL1 fusion nucleic acid molecule comprises a fusion between an ABL1 gene, or a portion thereof, fused to: (a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; (b) a second gene, or a portion of a second gene, wherein the ABL1 fusion nucleic acid molecule comprises or results from an ABL1 breakpoint within any of exons 1-4 and 10, or introns 4-5, of ABL1; (c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC); (d) a NUP214 gene, or a portion thereof, wherein the cancer is bone marrow leukemia lymphocytic chronic (CLL) or soft tissue liposarcoma; (e) an AIF1L gene, or a portion thereof, wherein the cancer is soft tissue sarcoma, or ovary serous carcinoma; (f) an RCSD1 gene, or a portion thereof, wherein the cancer is bone marrow leukemia lymphocytic chronic (CLL); or (g) an EXOSC2 gene, or a portion thereof, wherein the cancer is peritoneum adenocarcinoma; wherein detection of the ABL1 fusion nucleic acid molecule, or the ABL1 fusion polypeptide encoded by the ABL1 fusion nucleic acid molecule, in the sample identifies the individual as one who may benefit from a treatment comprising an ABL1-targeted therapy.

In another aspect, provided herein is a method of identifying one or more treatment options for an individual having a cancer, the method comprising: (1) detecting or acquiring knowledge of an ABL1 fusion nucleic acid molecule, or an ABL1 fusion polypeptide encoded by the ABL1 fusion nucleic acid molecule, in a sample from the individual, wherein the ABL1 fusion nucleic acid molecule comprises a fusion between an ABL1 gene, or a portion thereof, fused to: (a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; (b) a second gene, or a portion of a second gene, wherein the ABL1 fusion nucleic acid molecule comprises or results from an ABL1 breakpoint within any of exons 1-4 and 10, or introns 4-5, of ABL1; (c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC); (d) a NUP214 gene, or a portion thereof, wherein the cancer is bone marrow leukemia lymphocytic chronic (CLL) or soft tissue liposarcoma; (e) an AIF1L gene, or a portion thereof, wherein the cancer is soft tissue sarcoma, or ovary serous carcinoma; (f) an RCSD1 gene, or a portion thereof, wherein the cancer is bone marrow leukemia lymphocytic chronic (CLL); or (g) an EXOSC2 gene, or a portion thereof, wherein the cancer is peritoneum adenocarcinoma; and (2) generating a report comprising one or more treatment options identified for the individual based, at least in part, on detection or on acquiring knowledge of the ABL1 fusion nucleic acid molecule, or the ABL1 fusion polypeptide encoded by the ABL1 fusion nucleic acid molecule, in the sample, wherein the one or more treatment options comprise an ABL1-targeted therapy.

In another aspect, provided herein is a method of selecting a treatment for an individual having a cancer, comprising acquiring knowledge of an ABL1 fusion nucleic acid molecule, or an ABL1 fusion polypeptide encoded by the ABL1 fusion nucleic acid molecule, in a sample from the individual, wherein the ABL1 fusion nucleic acid molecule comprises a fusion between an ABL1 gene, or a portion thereof, fused to: (a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; (b) a second gene, or a portion of a second gene, wherein the ABL1 fusion nucleic acid molecule comprises or results from an ABL1 breakpoint within any of exons 1-4 and 10, or introns 4-5, of ABL1; (c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC); (d) a NUP214 gene, or a portion thereof, wherein the cancer is bone marrow leukemia lymphocytic chronic (CLL) or soft tissue liposarcoma; (e) an AIF1L gene, or a portion thereof, wherein the cancer is soft tissue sarcoma, or ovary serous carcinoma; (f) an RCSD1 gene, or a portion thereof, wherein the cancer is bone marrow leukemia lymphocytic chronic (CLL); or (g) an EXOSC2 gene, or a portion thereof, wherein the cancer is peritoneum adenocarcinoma; wherein responsive to the acquisition of said knowledge: (i) the individual is classified as a candidate to receive a treatment comprising an ABL1-targeted therapy; and/or (ii) the individual is identified as likely to respond to a treatment that comprises an ABL1-targeted therapy.

In another aspect, provided herein is a method of predicting survival of an individual having a cancer, or an individual having a cancer treated with a treatment comprising an ABL1-targeted therapy, the method comprising acquiring knowledge of an ABL1 fusion nucleic acid molecule, or an ABL1 fusion polypeptide encoded by the ABL1 fusion nucleic acid molecule, in a sample from the individual, wherein the ABL1 fusion nucleic acid molecule comprises a fusion between an ABL1 gene, or a portion thereof, fused to: (a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; (b) a second gene, or a portion of a second gene, wherein the ABL1 fusion nucleic acid molecule comprises or results from an ABL1 breakpoint within any of exons 1-4 and 10, or introns 4-5, of ABL1; (c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC); (d) a NUP214 gene, or a portion thereof, wherein the cancer is bone marrow leukemia lymphocytic chronic (CLL) or soft tissue liposarcoma; (e) an AIF1L gene, or a portion thereof, wherein the cancer is soft tissue sarcoma, or ovary serous carcinoma; (f) an RCSD1 gene, or a portion thereof, wherein the cancer is bone marrow leukemia lymphocytic chronic (CLL); or (g) an EXOSC2 gene, or a portion thereof, wherein the cancer is peritoneum adenocarcinoma; wherein responsive to the acquisition of said knowledge, the individual is predicted to have longer survival when treated with a treatment comprising an ABL1-targeted therapy, as compared to survival of an individual whose cancer does not comprise an ABL1 fusion nucleic acid molecule or an ABL1 fusion polypeptide.

In another aspect, provided herein is a method of treating or delaying progression of a cancer, comprising: (1) acquiring knowledge of an ABL1 fusion nucleic acid molecule, or an ABL1 fusion polypeptide encoded by the ABL1 fusion nucleic acid molecule, in a sample from an individual having a cancer, wherein the ABL1 fusion nucleic acid molecule comprises a fusion between an ABL1 gene, or a portion thereof, fused to: (a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; (b) a second gene, or a portion of a second gene, wherein the ABL1 fusion nucleic acid molecule comprises or results from an ABL1 breakpoint within any of exons 1-4 and 10, or introns 4-5, of ABL1; (c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC); (d) a NUP214 gene, or a portion thereof, wherein the cancer is bone marrow leukemia lymphocytic chronic (CLL) or soft tissue liposarcoma; (e) an AIF1L gene, or a portion thereof, wherein the cancer is soft tissue sarcoma, or ovary serous carcinoma; (f) an RCSD1 gene, or a portion thereof, wherein the cancer is bone marrow leukemia lymphocytic chronic (CLL); or (g) an EXOSC2 gene, or a portion thereof, wherein the cancer is peritoneum adenocarcinoma; and (2) responsive to said knowledge, administering to the individual an effective amount of a treatment that comprises an ABL1-targeted therapy.

In another aspect, provided herein is a method of treating or delaying progression of a cancer, comprising administering to an individual having a cancer an effective amount of a treatment that comprises an ABL1-targeted therapy, wherein the ABL1-targeted therapy is administered responsive to acquiring knowledge of an ABL1 fusion nucleic acid molecule, or an ABL1 fusion polypeptide encoded by the ABL1 fusion nucleic acid molecule, in a sample from the individual, wherein the ABL1 fusion nucleic acid molecule comprises a fusion between an ABL1 gene, or a portion thereof, fused to: (a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; (b) a second gene, or a portion of a second gene, wherein the ABL1 fusion nucleic acid molecule comprises or results from an ABL1 breakpoint within any of exons 1-4 and 10, or introns 4-5, of ABL1; (c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC); (d) a NUP214 gene, or a portion thereof, wherein the cancer is bone marrow leukemia lymphocytic chronic (CLL) or soft tissue liposarcoma; (e) an AIF1L gene, or a portion thereof, wherein the cancer is soft tissue sarcoma, or ovary serous carcinoma; (f) an RCSD1 gene, or a portion thereof, wherein the cancer is bone marrow leukemia lymphocytic chronic (CLL); or (g) an EXOSC2 gene, or a portion thereof, wherein the cancer is peritoneum adenocarcinoma.

In another aspect, provided herein is a method of monitoring, evaluating or screening an individual having a cancer, comprising acquiring knowledge of an ABL1 fusion nucleic acid molecule, or an ABL1 fusion polypeptide encoded by the ABL1 fusion nucleic acid molecule, in a sample from the individual, wherein the ABL1 fusion nucleic acid molecule comprises a fusion between an ABL1 gene, or a portion thereof, fused to: (a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; (b) a second gene, or a portion of a second gene, wherein the ABL1 fusion nucleic acid molecule comprises or results from an ABL1 breakpoint within any of exons 1-4 and 10, or introns 4-5, of ABL1; (c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC); (d) a NUP214 gene, or a portion thereof, wherein the cancer is bone marrow leukemia lymphocytic chronic (CLL) or soft tissue liposarcoma; (e) an AIF1L gene, or a portion thereof, wherein the cancer is soft tissue sarcoma, or ovary serous carcinoma; (f) an RCSD1 gene, or a portion thereof, wherein the cancer is bone marrow leukemia lymphocytic chronic (CLL); or (g) an EXOSC2 gene, or a portion thereof, wherein the cancer is peritoneum adenocarcinoma; wherein responsive to the acquisition of said knowledge, the individual is predicted to benefit from a treatment comprising an ABL1-targeted therapy and/or to have longer survival when treated with a treatment comprising an ABL1-targeted therapy, as compared to an individual whose cancer does not comprise an ABL1 fusion nucleic acid molecule or an ABL1 fusion polypeptide.

In another aspect, provided herein is a method of assessing an ABL1 fusion nucleic acid molecule, or an ABL1 fusion polypeptide, in a cancer in an individual, the method comprising: (1) detecting an ABL1 fusion nucleic acid molecule, or an ABL1 fusion polypeptide encoded by the ABL1 fusion nucleic acid molecule, in a sample from the individual, wherein the ABL1 fusion nucleic acid molecule comprises a fusion between an ABL1 gene, or a portion thereof, fused to: (a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; (b) a second gene, or a portion of a second gene, wherein the ABL1 fusion nucleic acid molecule comprises or results from an ABL1 breakpoint within any of exons 1-4 and 10, or introns 4-5, of ABL1; (c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC); (d) a NUP214 gene, or a portion thereof, wherein the cancer is bone marrow leukemia lymphocytic chronic (CLL) or soft tissue liposarcoma; (e) an AIF1L gene, or a portion thereof, wherein the cancer is soft tissue sarcoma, or ovary serous carcinoma; (f) an RCSD1 gene, or a portion thereof, wherein the cancer is bone marrow leukemia lymphocytic chronic (CLL); or (g) an EXOSC2 gene, or a portion thereof, wherein the cancer is peritoneum adenocarcinoma; and (2) providing an assessment of the ABL1 fusion nucleic acid molecule, or the ABL1 fusion polypeptide encoded by the ABL1 fusion nucleic acid molecule, in the sample.

In another aspect, provided herein is a method of detecting an ABL1 fusion nucleic acid molecule or an ABL1 fusion polypeptide in a cancer, the method comprising detecting an ABL1 fusion nucleic acid molecule, or an ABL1 fusion polypeptide encoded by the ABL1 fusion nucleic acid molecule, in a sample from an individual having a cancer, wherein the ABL1 fusion nucleic acid molecule comprises a fusion between an ABL1 gene, or a portion thereof, fused to: (a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; (b) a second gene, or a portion of a second gene, wherein the ABL1 fusion nucleic acid molecule comprises or results from an ABL1 breakpoint within any of exons 1-4 and 10, or introns 4-5, of ABL1; (c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC); (d) a NUP214 gene, or a portion thereof, wherein the cancer is bone marrow leukemia lymphocytic chronic (CLL) or soft tissue liposarcoma; (e) an AIF1L gene, or a portion thereof, wherein the cancer is soft tissue sarcoma, or ovary serous carcinoma; (f) an RCSD1 gene, or a portion thereof, wherein the cancer is bone marrow leukemia lymphocytic chronic (CLL); or (g) an EXOSC2 gene, or a portion thereof, wherein the cancer is peritoneum adenocarcinoma.

In another aspect, provided herein is a method of detecting the presence or absence of a cancer in an individual, the method comprising: (1) detecting the presence or absence of a cancer in a sample from the individual; and (2) detecting an ABL1 fusion nucleic acid molecule, or an ABL1 fusion polypeptide encoded by the ABL1 fusion nucleic acid molecule, in a sample from the individual, wherein the ABL1 fusion nucleic acid molecule comprises a fusion between an ABL1 gene, or a portion thereof, fused to: (a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; (b) a second gene, or a portion of a second gene, wherein the ABL1 fusion nucleic acid molecule comprises or results from an ABL1 breakpoint within any of exons 1-4 and 10, or introns 4-5, of ABL1; (c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC); (d) a NUP214 gene, or a portion thereof, wherein the cancer is bone marrow leukemia lymphocytic chronic (CLL) or soft tissue liposarcoma; (e) an AIF1L gene, or a portion thereof, wherein the cancer is soft tissue sarcoma, or ovary serous carcinoma; (f) an RCSD1 gene, or a portion thereof, wherein the cancer is bone marrow leukemia lymphocytic chronic (CLL); or (g) an EXOSC2 gene, or a portion thereof, wherein the cancer is peritoneum adenocarcinoma.

In some embodiments, which may be combined with any of the preceding aspects or embodiments, the method comprises detecting the presence of the cancer in a sample from the individual; and/or detecting the presence of the ABL1 fusion nucleic acid molecule, or the ABL1 fusion polypeptide encoded by the ABL1 fusion nucleic acid molecule, in a sample from the individual.

In another aspect, provided herein is a method for monitoring progression or recurrence of a cancer in an individual, the method comprising: (1) detecting, in a first sample obtained from the individual at a first time point, the presence or absence of an ABL1 fusion nucleic acid molecule, or an ABL1 fusion polypeptide encoded by the ABL1 fusion nucleic acid molecule; (2) detecting, in a second sample obtained from the individual at a second time point after the first time point, the presence or absence of an ABL1 fusion nucleic acid molecule, or an ABL1 fusion polypeptide encoded by the ABL1 fusion nucleic acid molecule; and (3) providing an assessment of cancer progression or cancer recurrence in the individual based, at least in part, on the presence or absence of the ABL1 fusion nucleic acid molecule, or the ABL1 fusion polypeptide encoded by the ABL1 fusion nucleic acid molecule, in the first sample and/or in the second sample; wherein the ABL1 fusion nucleic acid molecule comprises a fusion between an ABL1 gene, or a portion thereof, fused to: (a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; (b) a second gene, or a portion of a second gene, wherein the ABL1 fusion nucleic acid molecule comprises or results from an ABL1 breakpoint within any of exons 1-4 and 10, or introns 4-5, of ABL1; (c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC); (d) a NUP214 gene, or a portion thereof, wherein the cancer is bone marrow leukemia lymphocytic chronic (CLL) or soft tissue liposarcoma; (e) an AIF1L gene, or a portion thereof, wherein the cancer is soft tissue sarcoma, or ovary serous carcinoma; (f) an RCSD1 gene, or a portion thereof, wherein the cancer is bone marrow leukemia lymphocytic chronic (CLL); or (g) an EXOSC2 gene, or a portion thereof, wherein the cancer is peritoneum adenocarcinoma.

In some embodiments, which may be combined with any of the preceding aspects or embodiments, the presence of the ABL1 fusion nucleic acid molecule, or the ABL1 fusion polypeptide encoded by the ABL1 fusion nucleic acid molecule, in the first sample and/or in the second sample identifies the individual as having decreased risk of cancer progression or cancer recurrence when treated with a treatment comprising an ABL1-targeted therapy. In some embodiments, the method further comprises selecting a treatment, administering a treatment, adjusting a treatment, adjusting a dose of a treatment, or applying a treatment to the individual based, at least in part, on detecting the presence of the ABL1 fusion nucleic acid molecule, or the ABL1 fusion polypeptide encoded by the ABL1 fusion nucleic acid molecule, in the first sample and/or in the second sample, wherein the treatment comprises an ABL1-targeted therapy.

In another aspect, provided herein is a method of detecting an ABL1 fusion nucleic acid molecule, the method comprising: (a) providing a plurality of nucleic acid molecules obtained from a sample from an individual having a cancer, wherein the plurality of nucleic acid molecules comprises nucleic acid molecules corresponding to an ABL1 fusion nucleic acid molecule, wherein the ABL1 fusion nucleic acid molecule comprises a fusion between an ABL1 gene, or a portion thereof, fused to: (i) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; (ii) a second gene, or a portion of a second gene, wherein the ABL1 fusion nucleic acid molecule comprises or results from an ABL1 breakpoint within any of exons 1-4 and 10, or introns 4-5, of ABL1; (iii) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC); (iv) a NUP214 gene, or a portion thereof, wherein the cancer is bone marrow leukemia lymphocytic chronic (CLL) or soft tissue liposarcoma; (v) an AIF1L gene, or a portion thereof, wherein the cancer is soft tissue sarcoma, or ovary serous carcinoma; (vi) an RCSD1 gene, or a portion thereof, wherein the cancer is bone marrow leukemia lymphocytic chronic (CLL); or (vii) an EXOSC2 gene, or a portion thereof, wherein the cancer is peritoneum adenocarcinoma; (b) optionally, ligating one or more adapters onto one or more nucleic acid molecules from the plurality of nucleic acid molecules; (c) optionally, amplifying the one or more ligated nucleic acid molecules from the plurality of nucleic acid molecules; (d) optionally, capturing amplified nucleic acid molecules from the amplified nucleic acid molecules; (e) sequencing, by a sequencer, the captured nucleic acid molecules to obtain a plurality of sequence reads that represent the captured nucleic acid molecules, wherein one or more of the plurality of sequence reads correspond to the ABL1 fusion nucleic acid molecule; (f) analyzing the plurality of sequence reads for the presence or absence of the ABL1 fusion nucleic acid molecule; and (g) based on the analyzing step, detecting the presence or absence of the ABL1 fusion nucleic acid molecule in the sample.

In some embodiments, which may be combined with any of the preceding aspects or embodiments, the sequencer comprises a next-generation sequencer.

In another aspect, provided herein is a method of detecting an ABL1 fusion nucleic acid molecule, the method comprising: (a) providing a sample from an individual having a cancer, wherein the sample comprises a plurality of nucleic acid molecules; (b) preparing a nucleic acid sequencing library from the plurality of nucleic acid molecules in the sample; (c) amplifying said library; (d) selectively enriching for one or more nucleic acid molecules comprising nucleotide sequences corresponding to an ABL1 fusion nucleic acid molecule in said library to produce an enriched sample, wherein the an ABL1 fusion nucleic acid molecule comprises a fusion between an ABL1 gene, or a portion thereof, fused to: (i) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; (ii) a second gene, or a portion of a second gene, wherein the ABL1 fusion nucleic acid molecule comprises or results from an ABL1 breakpoint within any of exons 1-4 and 10, or introns 4-5, of ABL1; (iii) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC); (iv) a NUP214 gene, or a portion thereof, wherein the cancer is bone marrow leukemia lymphocytic chronic (CLL) or soft tissue liposarcoma; (v) an AIF1L gene, or a portion thereof, wherein the cancer is soft tissue sarcoma (NOS), or ovary serous carcinoma; (vi) an RCSD1 gene, or a portion thereof, wherein the cancer is bone marrow leukemia lymphocytic chronic (CLL); or (vii) an EXOSC2 gene, or a portion thereof, wherein the cancer is peritoneum adenocarcinoma; (e) sequencing the enriched sample, thereby producing a plurality of sequence reads; (f) analyzing the plurality of sequence reads for the presence or absence of the ABL1 fusion nucleic acid molecule; and (g) detecting, based on the analyzing step, the presence or absence of the ABL1 fusion nucleic acid molecule in the sample from the individual.

In some embodiments, which may be combined with any of the preceding aspects or embodiments, one or more adapters comprise amplification primers, flow cell adapter sequences, substrate adapter sequences, sample index sequences, or unique molecular identifier (UMI) sequences. In some embodiments, which may be combined with any of the preceding aspects or embodiments, the selectively enriching comprises: (a) combining one or more bait molecules with the library, thereby hybridizing the one or more bait molecules to one or more nucleic acid molecules comprising nucleotide sequences corresponding to the ABL1 fusion nucleic acid molecule, and producing nucleic acid hybrids; and (b) isolating the nucleic acid hybrids to produce the enriched sample. In some embodiments, the amplified nucleic acid molecules are captured by hybridization with one or more bait molecules. In some embodiments, the amplifying comprises performing a polymerase chain reaction (PCR) amplification technique, a non-PCR amplification technique, or an isothermal amplification technique. In some embodiments, the method further comprises selectively enriching for one or more nucleic acid molecules in the sample comprising nucleotide sequences corresponding to the ABL1 fusion nucleic acid molecule; wherein the selectively enriching produces an enriched sample. In some embodiments, the selectively enriching comprises: (a) combining one or more bait molecules with the sample, thereby hybridizing the one or more bait molecules to one or more nucleic acid molecules in the sample comprising nucleotide sequences corresponding to the ABL1 fusion nucleic acid molecule and producing nucleic acid hybrids; and (b) isolating the nucleic acid hybrids to produce the enriched sample. In some embodiments, one or more bait molecules comprise a capture nucleic acid molecule configured to hybridize to a nucleotide sequence corresponding to the ABL1 fusion nucleic acid molecule. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides. In some embodiments, one or more bait molecules are conjugated to an affinity reagent or to a detection reagent. In some embodiments, the affinity reagent is an antibody, an antibody fragment, or biotin, or wherein the detection reagent is a fluorescent marker. In some embodiments, the capture nucleic acid molecule comprises a DNA, RNA, or mixed DNA/RNA molecule. In some embodiments, the selectively enriching comprises amplifying the one or more nucleic acid molecules comprising nucleotide sequences corresponding to the ABL1 fusion nucleic acid molecule using a polymerase chain reaction (PCR) to produce an enriched sample. In some embodiments, the method further comprises sequencing the enriched sample. In some embodiments, the sequencing comprises use of a massively parallel sequencing (MPS) technique, whole genome sequencing (WGS), whole exome sequencing, targeted sequencing, direct sequencing, or a Sanger sequencing technique; optionally wherein the sequencing comprises a massively parallel sequencing technique, and the massively parallel sequencing technique comprises next-generation sequencing (NGS). In some embodiments, which may be combined with any of the preceding aspects or embodiments, the plurality of nucleic acid molecules comprises a mixture of cancer nucleic acid molecules and non-cancer nucleic acid molecules. In some embodiments, the cancer nucleic acid molecules are derived from a tumor portion of a heterogeneous tissue biopsy sample, and the non-cancer nucleic acid molecules are derived from a normal portion of the heterogeneous tissue biopsy sample. In some embodiments, the sample comprises a liquid biopsy sample, and wherein the cancer nucleic acid molecules are derived from a circulating tumor DNA (ctDNA) fraction of the liquid biopsy sample, and the non-cancer nucleic acid molecules are derived from a non-tumor fraction of the liquid biopsy sample.

In another aspect, provided herein is a method of identifying a candidate treatment for a cancer in an individual in need thereof, comprising: performing DNA sequencing on a sample obtained from the individual to determine a sequencing mutation profile, wherein the sequencing mutation profile identifies the presence or absence of an ABL1 fusion nucleic acid molecule in the sample, wherein the ABL1 fusion nucleic acid molecule comprises a fusion between an ABL1 gene, or a portion thereof, fused to: (a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; (b) a second gene, or a portion of a second gene, wherein the ABL1 fusion nucleic acid molecule comprises or results from an ABL1 breakpoint within any of exons 1-4 and 10, or introns 4-5, of ABL1; (c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC); (d) a NUP214 gene, or a portion thereof, wherein the cancer is bone marrow leukemia lymphocytic chronic (CLL) or soft tissue liposarcoma; (e) an AIF1L gene, or a portion thereof, wherein the cancer is soft tissue sarcoma, or ovary serous carcinoma; (f) an RCSD1 gene, or a portion thereof, wherein the cancer is bone marrow leukemia lymphocytic chronic (CLL); or (g) an EXOSC2 gene, or a portion thereof, wherein the cancer is peritoneum adenocarcinoma; and selecting a treatment for the individual based, at least in part, on the sequencing mutation profile, wherein the treatment comprises an ABL1-targeted therapy.

In some embodiments, which may be combined with any of the preceding aspects or embodiments, the presence of the ABL1 fusion nucleic acid molecule in the sample identifies the individual as one who may benefit from a treatment comprising an ABL1-targeted therapy; and/or predicts the individual to have longer survival when treated with a treatment comprising an ABL1-targeted therapy, as compared to survival of an individual whose cancer does not comprise an ABL1 fusion nucleic acid molecule. In some embodiments, the sequencing comprises use of a massively parallel sequencing (MPS) technique, whole genome sequencing (WGS), whole exome sequencing, targeted sequencing, direct sequencing, or a Sanger sequencing technique; optionally wherein the sequencing comprises a massively parallel sequencing technique, and the massively parallel sequencing technique comprises next generation sequencing (NGS). In some embodiments, the sequencing mutation profile identifies the presence or absence of a fragment of the ABL1 fusion nucleic acid molecule comprising a breakpoint or fusion junction.

In another aspect, provided herein is a method of treating or delaying progression of cancer, comprising: detecting in a sample from an individual having a cancer an ABL1 fusion nucleic acid molecule, or an ABL1 fusion polypeptide encoded by the ABL1 fusion nucleic acid molecule, wherein the ABL1 fusion nucleic acid molecule comprises a fusion between an ABL1 gene, or a portion thereof, fused to: (a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; (b) a second gene, or a portion of a second gene, wherein the ABL1 fusion nucleic acid molecule comprises or results from an ABL1 breakpoint within any of exons 1-4 and 10, or introns 4-5, of ABL1; (c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC); (d) a NUP214 gene, or a portion thereof, wherein the cancer is bone marrow leukemia lymphocytic chronic (CLL) or soft tissue liposarcoma; (e) an AIF1L gene, or a portion thereof, wherein the cancer is soft tissue sarcoma, or ovary serous carcinoma; (f) an RCSD1 gene, or a portion thereof, wherein the cancer is bone marrow leukemia lymphocytic chronic (CLL); or (g) an EXOSC2 gene, or a portion thereof, wherein the cancer is peritoneum adenocarcinoma; and administering to the individual an effective amount of a treatment that comprises an ABL1-targeted therapy.

In some embodiments, which may be combined with any of the preceding aspects or embodiments, the method comprises generating a report, wherein the report: (a) indicates the presence of the ABL1 fusion nucleic acid molecule, or the ABL1 fusion polypeptide encoded by the ABL1 fusion nucleic acid molecule, in the sample from the individual; and/or (b) indicates a treatment or one or more treatment options identified or selected for the individual based, at least in part, on the presence of the ABL1 fusion nucleic acid molecule, or the ABL1 fusion polypeptide encoded by the ABL1 fusion nucleic acid molecule, in the sample from the individual, wherein the treatment or the one or more treatment options comprise an ABL1-targeted therapy. In some embodiments, the method further comprises generating a molecular profile for the individual based, at least in part, on detecting or acquiring knowledge of the ABL1 fusion nucleic acid molecule, or the ABL1 fusion polypeptide encoded by the ABL1 fusion nucleic acid molecule, in the sample from the individual. In some embodiments, the molecular profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the molecular profile for the individual further comprises results from a nucleic acid sequencing-based test. In some embodiments, the method comprises selecting a treatment, administering a treatment, or applying a treatment to the individual based on the generated molecular profile, wherein the treatment comprises an ABL1-targeted therapy. In some embodiments, the method comprises generating a report, wherein the report comprises the molecular profile for the individual. In some embodiments, the report further comprises information on a treatment or one or more treatment options identified or selected for the individual based, at least in part, on the molecular profile for the individual, wherein the treatment or one or more treatment options comprise an ABL1-targeted therapy. In some embodiments, the method further comprises providing the report to the individual, a caregiver, a healthcare provider, a physician, an oncologist, an electronic medical record system, a hospital, a clinic, a third-party payer, an insurance company, or a government office.

In some embodiments, which may be combined with any of the preceding aspects or embodiments, individual is a human. In some embodiments, the method comprises obtaining the sample from the individual. In some embodiments, the sample is obtained or derived from the cancer. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample comprising blood, plasma, cerebrospinal fluid, sputum, stool, urine, or saliva. In some embodiments, the sample comprises cells and/or nucleic acids from the cancer. In some embodiments, the sample comprises mRNA, DNA, circulating tumor DNA (ctDNA), cell-free DNA, or cell-free RNA from the cancer. In some embodiments, the sample is a liquid biopsy sample comprising circulating tumor cells (CTCs). In some embodiments, the sample is a liquid biopsy sample comprising cell-free DNA (cfDNA), circulating tumor DNA (ctDNA), or any combination thereof.

In some embodiments, which may be combined with any of the preceding aspects or embodiments, the acquiring knowledge of the ABL1 fusion nucleic acid molecule, or the ABL1 fusion polypeptide encoded by the ABL1 fusion nucleic acid molecule, comprises detecting the ABL1 fusion nucleic acid molecule, or the ABL1 fusion polypeptide encoded by the ABL1 fusion nucleic acid molecule, in the sample. In some embodiments, detecting the ABL1 fusion nucleic acid molecule, or the ABL1 fusion polypeptide encoded by the ABL1 fusion nucleic acid molecule, in the sample comprises detecting a fragment of the ABL1 fusion nucleic acid molecule, or of the ABL1 fusion polypeptide encoded by the ABL1 fusion nucleic acid molecule, comprising a breakpoint or fusion junction. In some embodiments, the method comprises the detection in the sample of the ABL1 fusion nucleic acid by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing. In some embodiments, the sequencing comprises a massively parallel sequencing (MPS) technique, whole genome sequencing (WGS), whole exome sequencing, targeted sequencing, direct sequencing, or a Sanger sequencing technique; optionally wherein the massively parallel sequencing (MPS) technique comprises next-generation sequencing (NGS). In some embodiments, the ABL1 fusion polypeptide encoded by the ABL1 fusion nucleic acid molecule is detected in the sample by one or more of: immunoblotting, enzyme linked immunosorbent assay (ELISA), immunohistochemistry, or mass spectrometry.

In some embodiments, which may be combined with any of the preceding aspects or embodiments, the ABL1 fusion polypeptide encoded by the ABL1 fusion nucleic acid molecule comprises an ABL1 kinase domain, or a fragment of an ABL1 kinase domain, having a kinase activity; optionally wherein the kinase activity is constitutive.

In some embodiments, which may be combined with any of the preceding aspects or embodiments, the ABL1 fusion polypeptide encoded by the ABL1 fusion nucleic acid molecule is oncogenic; optionally wherein the ABL1 fusion polypeptide encoded by the ABL1 fusion nucleic acid molecule promotes cancer cell survival, angiogenesis, cancer cell proliferation, and any combination thereof.

In some embodiments, which may be combined with any of the preceding aspects or embodiments, the ABL1-targeted therapy comprises one or more of a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer comprising an ABL1 gene fusion or rearrangement, an ABL1-targeted therapy being tested in a clinical trial, a treatment for cancer comprising an ABL1 gene fusion or rearrangement being tested in a clinical trial, or any combination thereof. In some embodiments, the ABL1-targeted therapy is a kinase inhibitor. In some embodiments, the ABL1-targeted therapy is a tyrosine kinase inhibitor. In some embodiments, the ABL1-targeted therapy is kinase inhibitor that inhibits the kinase activity of an ABL1 polypeptide. In some embodiments, the ABL1-targeted therapy is a multi-kinase inhibitor or an ABL1-specific inhibitor. In some embodiments, the ABL1-targeted therapy comprises one or more of imatinib, nilotinib, dasatinib, ponatinib, bosutinib, asciminib, olverembatinib, radotinib or vodobatinib. In some embodiments, the nucleic acid inhibits the expression of the ABL1 fusion nucleic acid molecule, or the ABL1 fusion polypeptide encoded by the ABL1 fusion nucleic acid molecule. In some embodiments, the nucleic acid is a double-stranded RNA (dsRNA), a small interfering RNA (siRNA), or a small hairpin RNA (shRNA). In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In some embodiments, which may be combined with any of the preceding aspects or embodiments, the individual has received a prior anti-cancer treatment, or is being treated with an anti-cancer treatment; optionally wherein the cancer is resistant or refractory to the anti-cancer treatment. In some embodiments, the treatment or the one or more treatment options further comprise an additional anti-cancer therapy. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the nucleic acid comprises a double-stranded RNA (dsRNA), a small interfering RNA (siRNA), or a small hairpin RNA (shRNA).

In some embodiments, which may be combined with any of the preceding aspects or embodiments, the cancer is metastatic.

In some embodiments, which may be combined with any of the preceding aspects or embodiments, the ABL1 fusion nucleic acid molecule comprises a fusion between an ABL1 gene, or a portion thereof, fused to any one of genes APOOL, BMP2K, DNAH2, RHEX/C1orf186, EHMT1, MED27, RALGPS1, RFFL, SLC27A4, TSC1, TSPAN18, or ZNF804B, or a portion thereof, wherein the cancer is a carcinoma, a sarcoma, a lymphoma, a leukemia, a myeloma, a germ cell cancer, or a blastoma.

In some embodiments, which may be combined with any of the preceding aspects or embodiments, the ABL1 fusion nucleic acid molecule comprises a fusion between an ABL1 gene, or a portion thereof, fused to a second gene, or a portion of a second gene, wherein the ABL1 fusion nucleic acid molecule comprises or results from an ABL1 breakpoint within any of exons 1-4 and 10, or introns 4-5, of ABL1, wherein the cancer is a carcinoma, a sarcoma, a lymphoma, a leukemia, a myeloma, a germ cell cancer, or a blastoma.

In some embodiments, which may be combined with any of the preceding aspects or embodiments, the cancer is a solid tumor or a hematologic malignancy. In some embodiments, the cancer is a B cell cancer, multiple myeloma, bone marrow multiple myeloma, melanoma, breast cancer, lung cancer, bronchus cancer, colorectal cancer, prostate cancer, pancreatic cancer, stomach cancer, ovarian cancer, ovary epithelial carcinoma, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, testis germ cell tumor, biliary tract cancer, small bowel cancer, appendix cancer, salivary gland cancer, thyroid gland cancer, adrenal gland cancer, osteosarcoma, soft tissue osteosarcoma, chondrosarcoma, a cancer of hematological tissue, an adenocarcinoma, an inflammatory myofibroblastic tumor, a gastrointestinal stromal tumor (GIST), colon cancer, myelodysplastic syndrome (MDS), myeloproliferative disorder (MPD), acute lymphocytic leukemia (ALL) or bone marrow leukemia lymphocytic acute (ALL), bone marrow leukemia T cell acute (T-ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinoma, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, squamous cell cancer, non-squamous cell cancer, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, lung adenocarcinoma, non-small cell lung cancer, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, peritoneum adenocarcinoma, soft tissue malignant peripheral nerve sheath tumor (MPNST) or a carcinoid tumor.

In some embodiments, which may be combined with any of the preceding aspects or embodiments, the ABL1 fusion nucleic acid molecule comprises a fusion between an ABL1 gene, or a portion thereof, fused to an APOOL gene, or a portion thereof, and wherein the ABL1 fusion nucleic acid molecule: (a) comprises, in 5′ to 3′ direction, an APOOL gene, or a portion thereof, fused to an ABL1 gene, or a portion thereof; (b) comprises or results from a breakpoint within intron 2 of an APOOL gene, and/or exon 2 of an ABL1 gene; (c) comprises a fusion between exon 2, or a portion thereof, of an APOOL gene fused to exon 2, or a portion thereof, of an ABL1 gene; (d) comprises or results from a 5′ breakpoint within chromosomal coordinates chrX: 84302705-84302904 and/or a 3′ breakpoint within chromosomal coordinates chr9: 133729584-133729766; and/or (e) comprises, in the 5′ to 3′ direction, exon 1 and exon 2, or a portion thereof, of an APOOL gene, fused to exon 2, or a portion thereof, and exons 3-11 of an ABL1 gene; optionally, wherein the cancer is a lung adenocarcinoma. In some embodiments, the ABL1 fusion polypeptide encoded by the ABL1 fusion nucleic acid molecule comprises a fusion between an ABL1 polypeptide, or a portion thereof, fused to an APOOL polypeptide, or a portion thereof, and wherein the ABL1 fusion polypeptide: (a) comprises, in the N- to C-terminus direction, an APOOL polypeptide, or a portion thereof, fused to an ABL1 polypeptide, or a portion thereof; (b) results from a breakpoint within intron 2 of an APOOL gene, and/or exon 2 of an ABL1 gene; (c) comprises a fusion between an amino acid sequence encoded by exon 2, or a portion thereof, of an APOOL gene fused to an amino acid sequence encoded by exon 2, or a portion thereof, of an ABL1 gene; (d) results from a 5′ breakpoint within chromosomal coordinates chrX: 84302705-84302904 and/or a 3′ breakpoint within chromosomal coordinates chr9: 133729584-133729766; and/or (e) comprises, in the N- to C-terminus direction, an amino acid sequence encoded by exon 1 and exon 2, or a portion thereof, of APOOL, fused to an amino acid sequence encoded by exon 2, or a portion thereof, and exons 3-11 of ABL1; optionally, wherein the cancer is a lung adenocarcinoma.

In some embodiments, which may be combined with any of the preceding aspects or embodiments, the ABL1 fusion nucleic acid molecule comprises a fusion between an ABL1 gene, or a portion thereof, fused to an BMP2K gene, or a portion thereof, and wherein the ABL1 fusion nucleic acid molecule: (a) comprises, in 5′ to 3′ direction, a BMP2K gene, or a portion thereof, fused to an ABL1 gene, or a portion thereof; (b) comprises a fusion between exon 15, or a portion thereof, of a BMP2K gene fused to exon 3, or a portion thereof, of an ABL1 gene; (c) comprises or results from a 5′ breakpoint within chromosomal coordinates chr4: 79800022-79800062 and/or a 3′ breakpoint within chromosomal coordinates chr9: 133730205-133730245; and/or (d) comprises, in 5′ to 3′ direction, exons 1-14 and exon 15, or a portion thereof, of a BMP2K gene, fused to exon 3, or a portion thereof, and exons 4-11 of an ABL1 gene; optionally, wherein the cancer is a bone marrow leukemia T cell acute (T-ALL). In some embodiments, the ABL1 fusion polypeptide encoded by the ABL1 fusion nucleic acid molecule comprises a fusion between an ABL1 polypeptide, or a portion thereof, fused to a BMP2K polypeptide, or a portion thereof, and wherein the ABL1 fusion polypeptide: (a) comprises, in the N- to C-terminus direction, a BMP2K polypeptide, or a portion thereof, fused to an ABL1 polypeptide, or a portion thereof; (b) comprises a fusion between an amino acid sequence encoded by exon 15, or a portion thereof, of a BMP2K gene fused to an amino acid sequence encoded by exon 3, or a portion thereof, of an ABL1 gene; (c) results from a 5′ breakpoint within chromosomal coordinates chr4: 79800022-79800062 and/or a 3′ breakpoint within chromosomal coordinates chr9: 133730205-133730245; and/or (d) comprises, in the N- to C-terminus direction, an amino acid sequence encoded by exons 1-14 and exon 15, or a portion thereof, of a BMP2K gene, fused to an amino acid sequence encoded by exon 3, or a portion thereof, and exons 4-11 of an ABL1 gene; optionally, wherein the cancer is a bone marrow leukemia T cell acute (T-ALL).

In some embodiments, which may be combined with any of the preceding aspects or embodiments, the ABL1 fusion nucleic acid molecule comprises a fusion between an ABL1 gene, or a portion thereof, fused to a DNAH2 gene, or a portion thereof, and wherein the ABL1 fusion nucleic acid molecule: (a) comprises, in 5′ to 3′ direction, an ABL1 gene, or a portion thereof, fused to a DNAH2 gene, or a portion thereof; (b) comprises a fusion between exon 10, or a portion thereof, of an ABL1 gene fused to exon 4, or a portion thereof, of a DNAH2 gene; (c) comprises or results from a 5′ breakpoint within chromosomal coordinates chr9: 133755953-133755993 and/or a 3′ breakpoint within chromosomal coordinates chr17: 7636399-7636439; and/or (d) comprises, in 5′ to 3′ direction, exons 1-9 and exon 10, or a portion thereof, of an ABL1 gene, fused to exon 4, or a portion thereof, and exons 5-85 of a DNAH2 gene; optionally, wherein the cancer is a soft tissue sarcoma, and optionally wherein the soft tissue sarcoma is not otherwise specified (NOS). In some embodiments, the ABL1 fusion polypeptide encoded by the ABL1 fusion nucleic acid molecule comprises a fusion between an ABL1 polypeptide, or a portion thereof, fused to a DNAH2 polypeptide, or a portion thereof, and wherein the ABL1 fusion polypeptide: (a) comprises, in the N- to C-terminus direction, an ABL1 polypeptide, or a portion thereof, fused to a DNAH2 polypeptide, or a portion thereof; (b) comprises a fusion between an amino acid sequence encoded by exon 10, or a portion thereof, of an ABL1 gene fused to an amino acid sequence encoded by exon 4, or a portion thereof, of a DNAH2 gene; (c) results from a 5′ breakpoint within chromosomal coordinates chr9: 133755953-133755993 and/or a 3′ breakpoint within chromosomal coordinates chr17: 7636399-7636439; and/or (d) comprises, in the N- to C-terminus direction, an amino acid sequence encoded by exons 1-9 and exon 10, or a portion thereof, of an ABL1 gene, fused to an amino acid sequence encoded by exon 4, or a portion thereof, and exons 5-85 of a DNAH2 gene; optionally, wherein the cancer is a soft tissue sarcoma, and optionally wherein the soft tissue sarcoma is not otherwise specified (NOS).

In some embodiments, which may be combined with any of the preceding aspects or embodiments, the ABL1 fusion nucleic acid molecule comprises a fusion between an ABL1 gene, or a portion thereof, fused to a RHEX/Clorf186 gene, or a portion thereof, and wherein the ABL1 fusion nucleic acid molecule: (a) comprises, in 5′ to 3′ direction a RHEX/Clorf186 gene, or a portion thereof, fused to an ABL1 gene, or a portion thereof; (b) comprises a fusion between exon 1, or a portion thereof, of a RHEX/Clorf186 gene fused to exon 2, or a portion thereof, of an ABL1 gene; (c) comprises or results from a 5′ breakpoint within chromosomal coordinates chr1: 206288099-206288139 and/or a 3′ breakpoint within chromosomal coordinates chr9: 133729488-133729528; and/or (d) comprises, in 5′ to 3′ direction, exon 1, or a portion thereof, of an RHEX/Clorf186 gene, fused to exon 2, or a portion thereof, and exons 3-11 of ABL1; optionally, wherein the cancer is a bone marrow multiple myeloma. In some embodiments, the ABL1 fusion polypeptide encoded by the ABL1 fusion nucleic acid molecule comprises a fusion between an ABL1 polypeptide, or a portion thereof, fused to a RHEX/C1orf186 polypeptide, or a portion thereof, and wherein the ABL1 fusion polypeptide: (a) comprises, in the N- to C-terminus direction, a RHEX/C1orf186 polypeptide, or a portion thereof, fused to an ABL1 polypeptide, or a portion thereof; (b) comprises a fusion between an amino acid sequence encoded by exon 1, or a portion thereof, of a RHEX/C1orf186 gene fused to an amino acid sequence encoded by exon 2, or a portion thereof, of an ABL1 gene; (c) results from a 5′ breakpoint within chromosomal coordinates chr1: 206288099-206288139 and/or a 3′ breakpoint within chromosomal coordinates chr9: 133729488-133729528; and/or (d) comprises, in the N- to C-terminus direction, an amino acid sequence encoded by exon 1, or a portion thereof, of an RHEX/C1orf186 gene, fused to an amino acid sequence encoded by exon 2, or a portion thereof, and exons 3-11 of an ABL1 gene; optionally, wherein the cancer is a bone marrow multiple myeloma.

In some embodiments, which may be combined with any of the preceding aspects or embodiments, the ABL1 fusion nucleic acid molecule comprises a fusion between an ABL1 gene, or a portion thereof, fused to an EHMT1 gene, or a portion thereof, and wherein the ABL1 fusion nucleic acid molecule: (a) comprises, in 5′ to 3′ direction an EHMT1 gene, or a portion thereof, fused to an ABL1 gene, or a portion thereof; (b) comprises a fusion between exon 4, or a portion thereof, of an EHMT1 gene fused to exon 2, or a portion thereof, of an ABL1 gene; (c) comprises or results from a breakpoint within intron 4 of an EHMT1 gene and/or a breakpoint within intron 1 of an ABL1 gene; (d) comprises or results from a 5′ breakpoint within chromosomal coordinates chr9: 140630475-140630606 and/or a 3′ breakpoint within chromosomal coordinates chr9: 133729408-133729648; (e) comprises, in 5′ to 3′ direction, exons 1-3 and exon 4, or a portion thereof, of an EHMT1 gene, fused to exon 2, or a portion thereof, and exons 3-11 of an ABL1 gene; (f) comprises the nucleotide sequence of SEQ ID NO: 2, or a nucleotide sequence having at least about 70% homology thereto; and/or (g) encodes an ABL1 fusion polypeptide comprising the amino acid sequence of SEQ ID NO: 23, or an amino acid sequence having at least about 70% homology thereto; optionally, wherein the cancer is a lung adenocarcinoma. In some embodiments, the ABL1 fusion polypeptide encoded by the ABL1 fusion nucleic acid molecule comprises a fusion between an ABL1 polypeptide, or a portion thereof, fused to an EHMT1 polypeptide, or a portion thereof, and wherein the ABL1 fusion polypeptide: (a) comprises, in the N- to C-terminus direction, an EHMT1 polypeptide, or a portion thereof, fused to an ABL1 polypeptide, or a portion thereof; (b) comprises a fusion between an amino acid sequence encoded by exon 4, or a portion thereof, of an EHMT1 gene fused to an amino acid sequence encoded by exon 2, or a portion thereof, of an ABL1 gene; (c) results from a breakpoint within intron 4 of an EHMT1 gene and/or a breakpoint within intron 1 of an ABL1 gene; (d) results from a 5′ breakpoint within chromosomal coordinates chr9: 140630475-140630606 and/or a 3′ breakpoint within chromosomal coordinates chr9: 133729408-133729648; (e) comprises, in the N- to C-terminus direction, an amino acid sequence encoded by exons 1-3 and exon 4, or a portion thereof, of an EHMT1 gene, fused to an amino acid sequence encoded by exon 2, or a portion thereof, and exons 3-11 of an ABL1 gene; (f) is encoded by the nucleotide sequence of SEQ ID NO: 2, or a nucleotide sequence having at least about 70% homology thereto; and/or (g) comprises the amino acid sequence of SEQ ID NO: 23, or an amino acid sequence having at least about 70% homology thereto; optionally, wherein the cancer is a lung adenocarcinoma.

In some embodiments, which may be combined with any of the preceding aspects or embodiments, the ABL1 fusion nucleic acid molecule comprises a fusion between an ABL1 gene, or a portion thereof, fused to a MED27 gene, or a portion thereof, and wherein the ABL1 fusion nucleic acid molecule: (a) comprises, in 5′ to 3′ direction a MED27 gene, or a portion thereof, fused to an ABL1 gene, or a portion thereof; (b) comprises a fusion between exon 4, or a portion thereof, of a MED27 gene fused to exon 5, or a portion thereof, of an ABL1 gene; (c) comprises or results from a breakpoint within intron 4 of a MED27 gene and/or a breakpoint within intron 4 of an ABL1 gene; (d) comprises or results from a 5′ breakpoint within chromosomal coordinates chr9: 134774423-134776006 and/or a 3′ breakpoint within chromosomal coordinates chr9: 133747364-133747762; (e) comprises, in 5′ to 3′ direction, exons 1-3 and exon 4, or a portion thereof, of a MED27 gene fused to exon 5, or a portion thereof, and exons 6-11 of an ABL1 gene; (f) comprises the nucleotide sequence of SEQ ID NO: 3, or a nucleotide sequence having at least about 70% homology thereto; and/or (g) encodes an ABL1 fusion polypeptide comprising the amino acid sequence of SEQ ID NO: 24, or an amino acid sequence having at least about 70% homology thereto; optionally, wherein the cancer is an ovary epithelial carcinoma, and optionally wherein the ovary epithelial carcinoma is not otherwise specified (NOS). In some embodiments, the ABL1 fusion polypeptide encoded by the ABL1 fusion nucleic acid molecule comprises a fusion between an ABL1 polypeptide, or a portion thereof, fused to a MED27 polypeptide, or a portion thereof, and wherein the ABL1 fusion polypeptide: (a) comprises, in the N- to C-terminus direction, a MED27 polypeptide, or a portion thereof, fused to an ABL1 polypeptide, or a portion thereof; (b) comprises a fusion between an amino acid sequence encoded by exon 4, or a portion thereof, of a MED27 gene fused to an amino acid sequence encoded by exon 5, or a portion thereof, of an ABL1 gene; (c) results from a breakpoint within intron 4 of a MED27 gene and/or a breakpoint within intron 4 of an ABL1 gene; (d) results from a 5′ breakpoint within chromosomal coordinates chr9: 134774423-134776006 and/or a 3′ breakpoint within chromosomal coordinates chr9: 133747364-133747762; (e) comprises, in the N- to C-terminus direction, an amino acid sequence encoded by exons 1-3 and exon 4, or a portion thereof, of a MED27 gene, fused to an amino acid sequence encoded by exon 5, or a portion thereof, and exons 6-11 of an ABL1 gene; (f) is encoded by the nucleotide sequence of SEQ ID NO: 3, or a nucleotide sequence having at least about 70% homology thereto; and/or (g) comprises the amino acid sequence of SEQ ID NO: 24, or an amino acid sequence having at least about 70% homology thereto; optionally, wherein the cancer is an ovary epithelial carcinoma, and optionally wherein the ovary epithelial carcinoma is not otherwise specified (NOS).

In some embodiments, which may be combined with any of the preceding aspects or embodiments, the ABL1 fusion nucleic acid molecule comprises a fusion between an ABL1 gene, or a portion thereof, fused to a RALGPS1 gene, or a portion thereof, and wherein the ABL1 fusion nucleic acid molecule: (a) comprises, in 5′ to 3′ direction, a RALGPS1 gene, or a portion thereof, fused to an ABL1 gene, or a portion thereof; (b) comprises a fusion between exon 1, or a portion thereof, of a RALGPS1 gene fused to exon 3, or a portion thereof, of an ABL1 gene; (c) comprises or results from a breakpoint within intron 1 of a RALGPS1 gene and/or a breakpoint within intron 2 of an ABL1 gene; (d) comprises or results from a 5′ breakpoint within chromosomal coordinates chr9: 129694023-129694282 and/or a 3′ breakpoint within chromosomal coordinates chr9: 133730118-133730529; and/or (e) comprises, in 5′ to 3′ direction, exon 1, or a portion thereof of a RALGPS1 gene, fused to exon 3, or a portion thereof, and exons 4-11 of an ABL1 gene; optionally, wherein the cancer is melanoma, and optionally wherein the melanoma is unknown primary melanoma. In some embodiments, the ABL1 fusion polypeptide encoded by the ABL1 fusion nucleic acid molecule comprises a fusion between an ABL1 polypeptide, or a portion thereof, fused to a RALGPS1 polypeptide, or a portion thereof, and wherein the ABL1 fusion polypeptide: (a) comprises, in the N- to C-terminus direction, a RALGPS1 polypeptide, or a portion thereof, fused to an ABL1 polypeptide, or a portion thereof; (b) comprises a fusion between an amino acid sequence encoded by exon 1, or a portion thereof, of a RALGPS1 gene fused to an amino acid sequence encoded by exon 3, or a portion thereof, of an ABL1 gene; (c) results from a breakpoint within intron 1 of a RALGPS1 gene and/or a breakpoint within intron 2 of an ABL1 gene; (d) results from a 5′ breakpoint within chromosomal coordinates chr9: 129694023-129694282 and/or a 3′ breakpoint within chromosomal coordinates chr9: 133730118-133730529; and/or (e) comprises, in the N- to C-terminus direction, an amino acid sequence encoded by exon 1, or a portion thereof, of a RALGPS1 gene, fused to an amino acid sequence encoded by exon 3, or a portion thereof, and exons 4-11 of an ABL1 gene; optionally, wherein the cancer is melanoma, and optionally wherein the melanoma is unknown primary melanoma.

In some embodiments, which may be combined with any of the preceding aspects or embodiments, the ABL1 fusion nucleic acid molecule comprises a fusion between an ABL1 gene, or a portion thereof, fused to a RFFL gene, or a portion thereof, and wherein the ABL1 fusion nucleic acid molecule: (a) comprises, in 5′ to 3′ direction a RFFL gene, or a portion thereof, fused to an ABL1 gene, or a portion thereof; (b) comprises a fusion between exon 5, or a portion thereof, of a RFFL gene fused to exon 2, or a portion thereof, of an ABL1 gene; (c) comprises or results from a 5′ breakpoint within chromosomal coordinates chr17: 33343379-33343419 and/or a 3′ breakpoint within chromosomal coordinates chr9: 133729485-133729525; (d) comprises, in 5′ to 3′ direction, exons 1-4 and exon 5, or a portion thereof, of an RFFL gene fused to exon 2, or a portion thereof, and exons 3-11 of an ABL1 gene; (e) comprises the nucleotide sequence of SEQ ID NO: 4, or a nucleotide sequence having at least about 70% homology thereto; and/or (f) encodes an ABL1 fusion polypeptide comprising the amino acid sequence of SEQ ID NO: 25, or an amino acid sequence having at least about 70% homology thereto; optionally, wherein the cancer is a soft tissue malignant peripheral nerve sheath tumor (MPNST). In some embodiments, the ABL1 fusion polypeptide encoded by the ABL1 fusion nucleic acid molecule comprises a fusion between an ABL1 polypeptide, or a portion thereof, fused to a RFFL polypeptide, or a portion thereof, and wherein the ABL1 fusion polypeptide: (a) comprises, in the N- to C-terminus direction, a RFFL polypeptide, or a portion thereof, fused to an ABL1 polypeptide, or a portion thereof; (b) comprises a fusion between an amino acid sequence encoded by exon 5, or a portion thereof, of a RFFL gene fused to an amino acid sequence encoded by exon 2, or a portion thereof, of an ABL1 gene; (c) results from a 5′ breakpoint within chromosomal coordinates chr17: 33343379-33343419 and/or a 3′ breakpoint within chromosomal coordinates chr9: 133729485-133729525; (d) comprises, in the N- to C-terminus direction, an amino acid sequence encoded by exons 1-4 and exon 5, or a portion thereof, of an RFFL gene, fused to an amino acid sequence encoded by exon 2, or a portion thereof, and exons 3-11 of an ABL1 gene; (e) is encoded by the nucleotide sequence of SEQ ID NO: 4, or a nucleotide sequence having at least about 70% homology thereto; and/or (f) comprises the amino acid sequence of SEQ ID NO: 25, or an amino acid sequence having at least about 70% homology thereto; optionally, wherein the cancer is a soft tissue malignant peripheral nerve sheath tumor (MPNST).

In some embodiments, which may be combined with any of the preceding aspects or

embodiments, the ABL1 fusion nucleic acid molecule comprises a fusion between an ABL1 gene, or a portion thereof, fused to a SLC27A4 gene, or a portion thereof, and wherein the ABL1 fusion nucleic acid molecule: (a) comprises, in 5′ to 3′ direction, a SLC27A4 gene, or a portion thereof, fused to an ABL1 gene, or a portion thereof; (b) comprises a fusion between exon 12, or a portion thereof, of a SLC27A4 gene fused to exon 6, or a portion thereof, of an ABL1 gene; (c) comprises or results from a breakpoint within intron 12 of a SLC27A4 gene and/or a breakpoint within intron 5 of an ABL1 gene; (d) comprises or results from a 5′ breakpoint within chromosomal coordinates chr9: 131119603-131119801 and/or a 3′ breakpoint within chromosomal coordinates chr9: 133748211-133748501; (e) comprises, in 5′ to 3′ direction, exons 1-11 and exon 12, or a portion thereof, of a SLC27A4 gene, fused to exon 6, or a portion thereof, and exons 7-11 of an ABL1 gene; (f) comprises the nucleotide sequence of SEQ ID NO: 5, or a nucleotide sequence having at least about 70% homology thereto; and/or (g) encodes an ABL1 fusion polypeptide comprising the amino acid sequence of SEQ ID NO: 26, or an amino acid sequence having at least about 70% homology thereto; optionally, wherein the cancer is a testis germ cell tumor, and optionally wherein the testis germ cell tumor is a mixed testis germ cell tumor. In some embodiments, the ABL1 fusion polypeptide encoded by the ABL1 fusion nucleic acid molecule comprises a fusion between an ABL1 polypeptide, or a portion thereof, fused to a SLC27A4 polypeptide, or a portion thereof, and wherein the ABL1 fusion polypeptide: (a) comprises, in the N- to C-terminus direction, a SLC27A4 polypeptide, or a portion thereof, fused to an ABL1 polypeptide, or a portion thereof; (b) comprises a fusion between an amino acid sequence encoded by exon 12, or a portion thereof, of a SLC27A4 gene fused to an amino acid sequence encoded by exon 6, or a portion thereof, of an ABL1 gene; (c) results from a breakpoint within intron 12 of a SLC27A4 gene and/or a breakpoint within intron 5 of an ABL1 gene; (d) results from a 5′ breakpoint within chromosomal coordinates chr9: 131119603-131119801 and/or a 3′ breakpoint within chromosomal coordinates chr9: 133748211-133748501; (e) comprises, in the N- to C-terminus direction, an amino acid sequence encoded by exons 1-11 and exon 12, or a portion thereof, of an SLC27A4 gene, fused to an amino acid sequence encoded by exon 6, or a portion thereof, and exons 7-11 of an ABL1 gene; (f) is encoded by the nucleotide sequence of SEQ ID NO: 5, or a nucleotide sequence having at least about 70% homology thereto; and/or (g) comprises the amino acid sequence of SEQ ID NO: 26, or an amino acid sequence having at least about 70% homology thereto; optionally, wherein the cancer is a testis germ cell tumor, and optionally wherein the testis germ cell tumor is a mixed testis germ cell tumor.

In some embodiments, which may be combined with any of the preceding aspects or embodiments, the ABL1 fusion nucleic acid molecule comprises a fusion between an ABL1 gene, or a portion thereof, fused to a TSC1 gene, or a portion thereof, and wherein the ABL1 fusion nucleic acid molecule: (a) comprises, in 5′ to 3′ direction, a TSC1 gene, or a portion thereof, fused to an ABL1 gene, or a portion thereof; (b) comprises a fusion between exon 19, or a portion thereof, of a TSC1 gene fused to exon 2, or a portion thereof, of an ABL1 gene; (c) comprises or results from a 5′ breakpoint within chromosomal coordinates chr9:135,776,998-135,777,038 and/or a 3′ breakpoint within chromosomal coordinates chr9: 133729491-133729531; and/or (d) comprises, in 5′ to 3′ direction, exons 1-18 and exon 19, or a portion thereof, of a TSC1 gene, fused to exon 2, or a portion thereof, and exons 3-11 of an ABL1 gene; optionally, wherein the cancer is a soft tissue malignant peripheral nerve sheath tumor (MPNST). In some embodiments, the ABL1 fusion polypeptide encoded by the ABL1 fusion nucleic acid molecule comprises a fusion between an ABL1 polypeptide, or a portion thereof, fused to a TSC1 polypeptide, or a portion thereof, and wherein the ABL1 fusion polypeptide: (a) comprises, in the N- to C-terminus direction, a TSC1 polypeptide, or a portion thereof, fused to an ABL1 polypeptide, or a portion thereof; (b) comprises a fusion between an amino acid sequence encoded by exon 19, or a portion thereof, of a TSC1 gene fused to an amino acid sequence encoded by exon 2, or a portion thereof, of an ABL1 gene; (c) results from a 5′ breakpoint within chromosomal coordinates chr9:135,776,998-135,777,038 and/or a 3′ breakpoint within chromosomal coordinates chr9: 133729491-133729531; and/or (d) comprises, in 5′ to 3′ direction, an amino acid sequence encoded by exons 1-18 and exon 19, or a portion thereof, of a TSC1 gene, fused to an amino acid sequence encoded by exon 2, or a portion thereof, and exons 3-11 of an ABL1 gene; optionally, wherein the cancer is a soft tissue malignant peripheral nerve sheath tumor (MPNST).

In some embodiments, which may be combined with any of the preceding aspects or embodiments, the ABL1 fusion nucleic acid molecule comprises a fusion between an ABL1 gene, or a portion thereof, fused to a TSPAN18 gene, or a portion thereof, and wherein the ABL1 fusion nucleic acid molecule: (a) comprises, in 5′ to 3′ direction, a TSPAN18 gene, or a portion thereof, fused to an ABL1 gene, or a portion thereof; (b) comprises a fusion between exon 2, or a portion thereof, of a TSPAN18 gene fused to exon 2, or a portion thereof, of an ABL1 gene; (c) comprises or results from a 5′ breakpoint within chromosomal coordinates chr11: 44881929-44881969 and/or a 3′ breakpoint within chromosomal coordinates chr9: 133729517-133729557; and/or (d) comprises, in 5′ to 3′ direction, exon 1 and exon 2, or a portion thereof, of a TSPAN18 gene, fused to exon 2, or a portion thereof, and exons 3-11 of an ABL1 gene; optionally, wherein the cancer is a soft tissue osteosarcoma, and optionally wherein the soft tissue osteosarcoma is an extraskeletal soft tissue osteosarcoma. In some embodiments, the ABL1 fusion polypeptide encoded by the ABL1 fusion nucleic acid molecule comprises a fusion between an ABL1 polypeptide, or a portion thereof, fused to a TSPAN18 polypeptide, or a portion thereof, and wherein the ABL1 fusion polypeptide: (a) comprises, in the N- to C-terminus direction, a TSPAN18 polypeptide, or a portion thereof, fused to an ABL1 polypeptide, or a portion thereof; (b) comprises a fusion between an amino acid sequence encoded by exon 2, or a portion thereof, of a TSPAN18 gene fused to an amino acid sequence encoded by exon 2, or a portion thereof, of an ABL1 gene; (c) results from a 5′ breakpoint within chromosomal coordinates chr11: 44881929-44881969 and/or a 3′ breakpoint within chromosomal coordinates chr9: 133729517-133729557; and/or (d) comprises, in the N- to C-terminus direction, an amino acid sequence encoded by exon 1 and exon 2, or a portion thereof, of a TSPAN18 gene, fused to an amino acid sequence encoded by exon 2, or a portion thereof, and exons 3-11 of an ABL1 gene; optionally, wherein the cancer is a soft tissue osteosarcoma, and optionally wherein the soft tissue osteosarcoma is an extraskeletal soft tissue osteosarcoma.

In some embodiments, which may be combined with any of the preceding aspects or embodiments, the ABL1 fusion nucleic acid molecule comprises a fusion between an ABL1 gene, or a portion thereof, fused to a ZNF804B gene, or a portion thereof, and wherein the ABL1 fusion nucleic acid molecule: (a) comprises, in 5′ to 3′ direction a ZNF804B gene, or a portion thereof, fused to an ABL1 gene, or a portion thereof; (b) comprises a fusion between exon 1, or a portion thereof, of a ZNF804B gene fused to exon 2, or a portion thereof, of an ABL1 gene; (c) comprises or results from a breakpoint within intron 1 of a ZNF804B gene and/or a breakpoint within intron 1 of an ABL1 gene; (d) comprises or results from a 5′ breakpoint within chromosomal coordinates chr7: 88521265-88521397 and/or a 3′ breakpoint within chromosomal coordinates chr9: 133729414-133729519; and/or (e) comprises, in 5′ to 3′ direction, exon 1, or a portion thereof, of a ZNF804B gene, fused to exon 2, or a portion thereof, and exons 3-11 of an ABL1 gene; optionally, wherein the cancer is a melanoma, optionally wherein the melanoma is a skin melanoma. In some embodiments, the ABL1 fusion polypeptide encoded by the ABL1 fusion nucleic acid molecule comprises a fusion between an ABL1 polypeptide, or a portion thereof, fused to a ZNF804B polypeptide, or a portion thereof, and wherein the ABL1 fusion polypeptide: (a) comprises, in the N- to C-terminus direction, a ZNF804B polypeptide, or a portion thereof, fused to an ABL1 polypeptide, or a portion thereof; (b) comprises a fusion between an amino acid sequence encoded by exon 1, or a portion thereof, of a ZNF804B gene fused to an amino acid sequence encoded by exon 2, or a portion thereof, of an ABL1 gene; (c) results from a breakpoint within intron 1 of a ZNF804B gene and/or a breakpoint within intron 1 of an ABL1 gene; (d) results from a 5′ breakpoint within chromosomal coordinates chr7: 88521265-88521397 and/or a 3′ breakpoint within chromosomal coordinates chr9: 133729414-133729519; and/or (e) comprises, in the N- to C-terminus, an amino acid sequence encoded by exon 1, or a portion thereof, of a ZNF804B gene, fused to an amino acid sequence encoded by exon 2, or a portion thereof, and exons 3-11 of an ABL1 gene; optionally, wherein the cancer is a melanoma, optionally wherein the melanoma is a skin melanoma.

In some embodiments, which may be combined with any of the preceding aspects or embodiments, the ABL1 fusion nucleic acid molecule comprises a fusion between an ABL1 gene, or a portion thereof, fused to a second gene, or a portion thereof, wherein the ABL1 fusion nucleic acid molecule comprises or results from an ABL1 breakpoint within exon 1 of ABL1, and wherein the ABL1 fusion nucleic acid molecule: (a) comprises, in 5′ to 3′ direction, the second gene, or a portion thereof, fused to exon 1 of an ABL1 gene, or a portion thereof; (b) comprises a fusion between an exon or an intron, or a portion thereof, of the second gene fused to exon 1, or a portion thereof, of an ABL1 gene; (c) comprises or results from a breakpoint within chromosomal coordinates chr9: 133589786-133589985; and/or (d) comprises, in 5′ to 3′ direction, one or more exons, or portions thereof, of the second gene, fused to exon 1, or a portion thereof, and exons 2-11 of ABL1; optionally wherein the cancer is a peritoneum adenocarcinoma. In some embodiments, the ABL1 fusion polypeptide encoded by the ABL1 fusion nucleic acid molecule comprises a fusion between an ABL1 polypeptide, or a portion thereof, fused to a polypeptide encoded by a second gene, or a portion thereof, wherein the ABL1 fusion polypeptide results from an ABL1 breakpoint within exon 1 of an ABL1 gene, and wherein the ABL1 fusion polypeptide: (a) comprises, in the N- to C-terminus direction, an amino acid sequence encoded by the second gene, or a portion thereof, fused to an amino acid sequence encoded by exon 1 of an ABL1 gene, or a portion thereof; (b) comprises a fusion between an amino acid sequence encoded by an exon, or a portion thereof, of the second gene fused to an amino acid sequence encoded by exon 1, or a portion thereof, of an ABL1 gene; (c) results from a breakpoint within chromosomal coordinates chr9: 133589786-133589985; and/or (d) comprises, in the N- to C-terminus direction, an amino acid sequence encoded by one or more exons, or portions thereof, of the second gene, fused to an amino acid sequence encoded by exon 1, or a portion thereof, and exons 2-11 of ABL1; optionally wherein the cancer is a peritoneum adenocarcinoma. In some embodiments, the second gene is an EXOSC2 gene, and the ABL1 fusion nucleic acid molecule: (a) comprises a fusion between exon 9, or a portion thereof, of an EXOSC2 gene fused to exon 1, or a portion thereof, of an ABL1 gene; (b) comprises or results from a breakpoint within exon 9 of an EXOSC2 gene and a breakpoint within exon 1 of an ABL1 gene; (c) comprises or results from a 5′ breakpoint within chromosomal coordinates chr9: 133579154-133579531 and/or a 3′ breakpoint within chromosomal coordinates chr9: 133589786-133589985; and/or (d) comprises, in 5′ to 3′ direction, exons 1-8 and exon 9, or a portion thereof, of an EXOSC2 gene, fused to exon 1, or a portion thereof, and exons 2-11 of an ABL1 gene; optionally, wherein the cancer is a peritoneum adenocarcinoma. In some embodiments, the second gene is an EXOSC2 gene, and the ABL1 fusion polypeptide: (a) comprises a fusion between an amino acid sequence encoded by exon 9, or a portion thereof, of an EXOSC2 gene fused to an amino acid sequence encoded by exon 1, or a portion thereof, of an ABL1 gene; (b) results from a breakpoint within exon 9 of an EXOSC2 gene and a breakpoint within exon 1 of an ABL1 gene; (c) results from a 5′ breakpoint within chromosomal coordinates chr9: 133579154-133579531 and/or a 3′ breakpoint within chromosomal coordinates chr9: 133589786-133589985; and/or (d) comprises, in the N- to C-terminus direction, an amino acid sequence encoded by exons 1-8 and exon 9, or a portion thereof, of an EXOSC2 gene, fused to an amino acid sequence encoded by exon 1, or a portion thereof, and exons 2-11 of an ABL1 gene; optionally, wherein the cancer is a peritoneum adenocarcinoma.

In some embodiments, which may be combined with any of the preceding aspects or embodiments, the ABL1 fusion nucleic acid molecule comprises a fusion between an ABL1 gene, or a portion thereof, fused to a second gene, or a portion thereof, wherein the ABL1 fusion nucleic acid molecule comprises or results from an ABL1 breakpoint within exon 2 of ABL1, and wherein the ABL1 fusion nucleic acid molecule: (a) comprises, in 5′ to 3′ direction, the second gene, or a portion thereof, fused to exon 2, or a portion thereof, of an ABL1 gene; (b) comprises a fusion between an exon or an intron, or a portion thereof, of the second gene fused to exon 2, or a portion thereof, of an ABL1 gene; (c) comprises or results from a breakpoint within chromosomal coordinates chr9: 133729584-133729766; and/or (d) comprises, in 5′ to 3′ direction, one or more exons, or portions thereof, of the second gene, fused to exon 2, or a portion thereof, and exons 3-11 of an ABL1 gene; optionally wherein the cancer is a lung cancer, and optionally wherein the lung cancer is a lung adenocarcinoma. In some embodiments, the ABL1 fusion polypeptide encoded by the ABL1 fusion nucleic acid molecule comprises a fusion between an ABL1 polypeptide, or a portion thereof, fused to a polypeptide encoded by a second gene, or a portion thereof, wherein the ABL1 fusion polypeptide results from an ABL1 breakpoint within exon 2 of ABL1, and wherein the ABL1 fusion polypeptide: (a) comprises, in the N- to C-terminus direction, an amino acid sequence encoded by the second gene, or a portion thereof, fused to an amino acid sequence encoded by exon 2, or a portion thereof, of an ABL1 gene; (b) comprises a fusion between an amino acid sequence encoded by an exon, or a portion thereof, of the second gene fused to an amino acid sequence encoded by exon 2, or a portion thereof, of an ABL1 gene; (c) results from a breakpoint within chromosomal coordinates chr9: 133729584-133729766; and/or (d) comprises, in the N- to C-terminus direction, an amino acid sequence encoded by one or more exons, or portions thereof, of the second gene, fused to an amino acid sequence encoded by exon 2, or a portion thereof, and exons 3-11 of an ABL1 gene; optionally wherein the cancer is a lung cancer, and optionally wherein the lung cancer is a lung adenocarcinoma. In some embodiments, the second gene is an APOOL gene, and the ABL1 fusion nucleic acid molecule: (a) comprises or results from a breakpoint within intron 2 of an APOOL gene, and exon 2 of ABL1; (b) comprises a fusion between exon 2, or a portion thereof, of an APOOL gene fused to exon 2, or a portion thereof, of an ABL1 gene; (c) comprises or results from a 5′ breakpoint within chromosomal coordinates chrX: 84302705-84302904 and/or 3′ breakpoint within chromosomal coordinates chr9: 133729584-133729766; and/or (d) comprises, in 5′ to 3′ direction, exon 1 and exon 2, or a portion thereof of an APOOL gene, fused to exon 2, or a portion thereof, and exons 3-11 of an ABL1 gene; optionally, wherein the cancer is a lung cancer, and optionally wherein the lung cancer is a lung adenocarcinoma. In some embodiments, the second gene is an APOOL gene, and the ABL1 fusion polypeptide: (a) results from a breakpoint within intron 2 of an APOOL gene, and exon 2 of an ABL1 gene; (b) comprises a fusion between an amino acid sequence encoded by exon 2, or a portion thereof, of an APOOL gene fused to an amino acid sequence encoded by exon 2, or a portion thereof, of an ABL1 gene; (c) results from a 5′ breakpoint within chromosomal coordinates chrX: 84302705-84302904 and/or 3′ breakpoint within chromosomal coordinates chr9: 133729584-133729766; and/or (d) comprises, in the N- to C-terminus direction, an amino acid sequence encoded by exon 1 and exon 2, or a portion thereof of an APOOL gene, fused to an amino acid sequence encoded by exon 2, or a portion thereof, and exons 3-11 of an ABL1 gene; optionally, wherein the cancer is a lung cancer, and optionally wherein the lung cancer is a lung adenocarcinoma.

In some embodiments, which may be combined with any of the preceding aspects or embodiments, the ABL1 fusion nucleic acid molecule comprises a fusion between an ABL1 gene, or a portion thereof, fused to a second gene, or a portion thereof, wherein the ABL1 fusion nucleic acid molecule comprises or results from an ABL1 breakpoint within exon 3 of ABL1, and wherein the ABL1 fusion nucleic acid molecule: (a) comprises, in 5′ to 3′ direction, the second gene, or a portion thereof, fused to exon 3, or a portion thereof, of an ABL1 gene; (b) comprises a fusion between an exon or an intron, or a portion thereof, of the second gene fused to exon 3, or a portion thereof, of an ABL1 gene; (c) comprises or results from a breakpoint within chromosomal coordinates chr9: 133730205-133730245, chr9: 133730195-133730235, chr9: 133730203-133730243, chr9: 133730234-133730274 or chr9: 133730218-133730258; and/or (d) comprises, in 5′ to 3′ direction, one or more exons, or portions thereof, of the second gene, fused to exon 3, or a portion thereof, and exons 4-11 of an ABL1 gene; optionally wherein the cancer is a bone marrow leukemia, optionally wherein the bone marrow leukemia is a bone marrow leukemia lymphocytic acute (ALL), a bone marrow leukemia B cell acute (B-ALL), or a bone marrow leukemia T cell acute (T-ALL). In some embodiments, the ABL1 fusion polypeptide encoded by the ABL1 fusion nucleic acid molecule comprises a fusion between an ABL1 polypeptide, or a portion thereof, fused to a polypeptide encoded by a second gene, or a portion thereof, wherein the ABL1 fusion polypeptide results from an ABL1 breakpoint within exon 3 of an ABL1 gene, and the ABL1 fusion polypeptide: (a) comprises, in the N- to C-terminus direction, an amino acid sequence encoded by the second gene, or a portion thereof, fused to an amino acid sequence encoded by exon 3, or a portion thereof, of an ABL1 gene; (b) comprises a fusion between an amino acid sequence encoded by an exon, or a portion thereof, of the second gene fused to an amino acid sequence encoded by exon 3, or a portion thereof, of an ABL1 gene; (c) results from a breakpoint within chromosomal coordinates chr9: 133730205-133730245, chr9: 133730195-133730235, chr9: 133730203-133730243, chr9: 133730234-133730274 or chr9: 133730218-133730258; and/or (d) comprises, in the N- to C-terminus direction, an amino acid sequence encoded by one or more exons, or portions thereof, of the second gene, fused to an amino acid sequence encoded by exon 3, or a portion thereof, and exons 4-11 of an ABL1 gene; optionally wherein the cancer is a bone marrow leukemia, optionally wherein the bone marrow leukemia is a bone marrow leukemia lymphocytic acute (ALL), a bone marrow leukemia B cell acute (B-ALL), or a bone marrow leukemia T cell acute (T-ALL). In some embodiments, the second gene is a NUP214 gene, and the ABL1 fusion nucleic acid molecule: (a) comprises a fusion between exon 34, or a portion thereof, of a NUP214 gene fused to exon 3, or a portion thereof, of an ABL1 gene; (b) comprises or results from a breakpoint within intron 34 of a NUP214 gene and a breakpoint within exon 3 of an ABL1 gene; (c) comprises or results from: a 5′ breakpoint within chromosomal coordinates chr9: 134106080-134106120 and/or a 3′ breakpoint within chromosomal coordinates chr9: 133730195-133730235, a 5′ breakpoint within chromosomal coordinates chr9: 134106030-134106070 and/or a 3′ breakpoint within chromosomal coordinates chr9: 133730218-133730258, a 5′ breakpoint within chromosomal coordinates chr9: 134106046-134106086 and/or a 3′ breakpoint within chromosomal coordinates chr9: 133730203-133730243, or a 5′ breakpoint within chromosomal coordinates chr9: 134106028-134106068 and/or a 3′ breakpoint within chromosomal coordinates chr9: 133730234-133730274; and/or (d) comprises, in 5′ to 3′ direction, exons 1-33 and exon 34, or a portion thereof, of a NUP214 gene, fused to exon 3, or a portion thereof, and exons 4-11 of an ABL1 gene; optionally, wherein the cancer is a bone marrow leukemia, and optionally wherein the bone marrow leukemia is a bone marrow leukemia B cell acute (B-ALL) or a bone marrow leukemia lymphocytic acute (ALL). In some embodiments, the second gene is a BMP2K gene, and the ABL1 fusion nucleic acid molecule: (a) comprises a fusion between exon 15, or a portion thereof, of a BMP2K gene fused to exon 3, or a portion thereof, of an ABL1 gene; (b) comprises or results from a 5′ breakpoint within chromosomal coordinates chr4: 79800022-79800062 and/or a 3′ breakpoint within chromosomal coordinates chr9: 133730205-133730245; and/or (c) comprises, in 5′ to 3′ direction, exons 1-14 and exon 15, or a portion thereof, of a BMP2K gene, fused to exon 3, or a portion thereof, and exons 4-11 of an ABL1 gene; optionally, wherein the cancer is a bone marrow leukemia, and optionally wherein the bone marrow leukemia is a bone marrow leukemia T cell acute (T-ALL). In some embodiments, the second gene is a BCR gene, and the ABL1 fusion nucleic acid molecule: (a) comprises a fusion between exon 1, or a portion thereof, of a BCR gene fused to exon 3, or a portion thereof, of an ABL1 gene; (b) comprises or results from a 5′ breakpoint within chromosomal coordinates chr22: 23524272-23524312 and/or a 3′ breakpoint within chromosomal coordinates chr9: 133730218-133730258; and/or (c) comprises, in 5′ to 3′ direction, exon 1, or a portion thereof of a BCR gene, fused to exon 3, or a portion thereof, and exons 4-11 of an ABL1 gene; optionally, wherein the cancer is a bone marrow leukemia, and optionally wherein the bone marrow leukemia is a bone marrow leukemia lymphocytic acute (ALL). In some embodiments, the second gene is a NUP214 gene, and the ABL1 fusion polypeptide: (a) comprises a fusion between an amino acid sequence encoded by exon 34, or a portion thereof, of a NUP214 gene fused to an amino acid sequence encoded by exon 3, or a portion thereof, of an ABL1 gene; (b) results from a breakpoint within intron 34 of a NUP214 gene and a breakpoint within exon 3 of an ABL1 gene; (c) results from: a 5′ breakpoint within chromosomal coordinates chr9: 134106080-134106120 and/or a 3′ breakpoint within chromosomal coordinates chr9: 133730195-133730235, a 5′ breakpoint within chromosomal coordinates chr9: 134106030-134106070 and/or a 3′ breakpoint within chromosomal coordinates chr9: 133730218-133730258, a 5′ breakpoint within chromosomal coordinates chr9: 134106046-134106086 and/or a 3′ breakpoint within chromosomal coordinates chr9: 133730203-133730243, or a 5′ breakpoint within chromosomal coordinates chr9: 134106028-134106068 and/or a 3′ breakpoint within chromosomal coordinates chr9: 133730234-133730274; and/or (d) comprises, in the N- to C-terminus direction, an amino acid sequence encoded by exons 1-33 and exon 34, or a portion thereof, of a NUP214 gene, fused to an amino acid sequence encoded by exon 3, or a portion thereof, and exons 4-11 of an ABL1 gene; optionally, wherein the cancer is a bone marrow leukemia, and optionally wherein the bone marrow leukemia is a bone marrow leukemia B cell acute (B-ALL) or a bone marrow leukemia lymphocytic acute (ALL). In some embodiments, the second gene is a BMP2K gene, and the ABL1 fusion polypeptide: (a) comprises a fusion between an amino acid sequence encoded by exon 15, or a portion thereof, of a BMP2K gene fused to an amino acid sequence encoded by exon 3, or a portion thereof, of an ABL1 gene; (b) results from a 5′ breakpoint within chromosomal coordinates chr4: 79800022-79800062 and/or a 3′ breakpoint within chromosomal coordinates chr9: 133730205-133730245; and/or (c) comprises, in the N- to C-terminus direction, an amino acid sequence encoded by exons 1-14 and exon 15, or a portion thereof, of a BMP2K gene, fused to an amino acid sequence encoded by exon 3, or a portion thereof, and exons 4-11 of an ABL1 gene; optionally, wherein the cancer is a bone marrow leukemia, and optionally wherein the bone marrow leukemia is a bone marrow leukemia T cell acute (T-ALL). In some embodiments, the second gene is a BCR gene, and the ABL1 fusion polypeptide: (a) comprises a fusion between an amino acid sequence encoded by exon 1, or a portion thereof, of a BCR gene fused to an amino acid sequence encoded by exon 3, or a portion thereof, of an ABL1 gene; (b) results from a 5′ breakpoint within chromosomal coordinates chr22: 23524272-23524312 and/or a 3′ breakpoint within chromosomal coordinates chr9: 133730218-133730258; and/or (c) comprises, in the N- to C-terminus direction, an amino acid sequence encoded by exon 1, or a portion thereof, of a BCR gene, fused to an amino acid sequence encoded by exon 3, or a portion thereof, and exons 4-11 of an ABL1 gene; optionally, wherein the cancer is a bone marrow leukemia, and optionally wherein the bone marrow leukemia is a bone marrow leukemia lymphocytic acute (ALL).

In some embodiments, which may be combined with any of the preceding aspects or embodiments, the ABL1 fusion nucleic acid molecule comprises a fusion between an ABL1 gene, or a portion thereof, fused to a second gene, or a portion thereof, wherein the ABL1 fusion nucleic acid molecule comprises or results from an ABL1 breakpoint within exon 4 of ABL1, and wherein the ABL1 fusion nucleic acid molecule: (a) comprises, in 5′ to 3′ direction, the second gene, or a portion thereof, fused to exon 4, or a portion thereof, of an ABL1 gene; (b) comprises a fusion between an exon or an intron, or a portion thereof, of the second gene fused to exon 4, or a portion thereof, of an ABL1 gene; (c) comprises or results from a breakpoint within chromosomal coordinates chr9: 133738173-133738213, chr9: 133738222-133738262, or chr9: 133738214-133738254; and/or (d) comprises, in 5′ to 3′ direction, one or more exons, or portions thereof, of the second gene, fused to exon 4, or a portion thereof, and exons 5-11 of an ABL1 gene; optionally wherein the cancer is a leukemia, optionally wherein the leukemia is a bone marrow leukemia B cell acute (B-ALL), a B-lymphoblastic leukemia-lymphoma (B-ALL), or a bone marrow leukemia lymphocytic chronic (CLL). In some embodiments, the ABL1 fusion polypeptide encoded by the ABL1 fusion nucleic acid molecule comprises a fusion between an ABL1 polypeptide, or a portion thereof, fused to a polypeptide encoded by a second gene, or a portion thereof, wherein the ABL1 fusion polypeptide comprises or results from an ABL1 breakpoint within exon 4 of ABL1, and wherein the ABL1 fusion polypeptide: (a) comprises, in the N- to C-terminus direction, an amino acid sequence encoded by the second gene, or a portion thereof, fused to an amino acid sequence encoded by exon 4, or a portion thereof, of an ABL1 gene; (b) comprises a fusion between an amino acid sequence encoded by an exon, or a portion thereof, of the second gene fused to an amino acid sequence encoded by exon 4, or a portion thereof, of an ABL1 gene; (c) results from a breakpoint within chromosomal coordinates chr9: 133738173-133738213, chr9: 133738222-133738262, or chr9: 133738214-133738254; and/or (d) comprises, in the N- to C-terminus direction, an amino acid sequence encoded by one or more exons, or portions thereof, of the second gene, fused to an amino acid sequence encoded by exon 4, or a portion thereof, and exons 5-11 of ABL1; optionally wherein the cancer is a leukemia, optionally wherein the leukemia is a bone marrow leukemia B cell acute (B-ALL), a B-lymphoblastic leukemia-lymphoma (B-ALL), or a bone marrow leukemia lymphocytic chronic (CLL). In some embodiments, the second gene is an RCSD1 gene, and the ABL1 fusion nucleic acid molecule: (a) comprises a fusion between exon 4, or a portion thereof, of an RCSD1 gene fused to exon 4, or a portion thereof, of an ABL1 gene; (b) comprises or results from a 5′ breakpoint within chromosomal coordinates chr1: 167654668-167654708 and/or a 3′ breakpoint within chromosomal coordinates chr9: 133738173-133738213; (c) comprises, in 5′ to 3′ direction, exons 1-3 and exon 4, or a portion thereof, of an RCSD1 gene, fused to exon 4, or a portion thereof, and exons 5-11 of an ABL1 gene; (d) comprises the nucleotide sequence of SEQ ID NO: 21, or a nucleotide sequence having at least about 70% homology thereto; and/or (e) encodes an ABL1 fusion polypeptide comprising the amino acid sequence of SEQ ID NO: 42, or an amino acid sequence having at least about 70% homology thereto; optionally, wherein the cancer is a bone marrow leukemia, and optionally wherein the bone marrow leukemia is a bone marrow leukemia lymphocytic chronic (CLL). In some embodiments, the second gene is an SNX2 gene, and the ABL1 fusion nucleic acid molecule: (a) comprises a fusion between exon 3, or a portion thereof, of an SNX2 gene fused to exon 4, or a portion thereof, of an ABL1 gene; (b) comprises or results from: a 5′ breakpoint within chromosomal coordinates chr5: 122135451-122135491 and/or a 3′ breakpoint within chromosomal coordinates chr9: 133738222-133738262, or a 5′ breakpoint within chromosomal coordinates chr5: 122135445-122135485 and/or a 3′ breakpoint within chromosomal coordinates chr9: 133738214-133738254; and/or (c) comprises, in 5′ to 3′ direction, exons 1-2 and exon 3, or a portion thereof of an SNX2 gene, fused to exon 4, or a portion thereof, and exons 5-11 of an ABL1 gene; optionally wherein the cancer is a leukemia, optionally wherein the leukemia is a bone marrow leukemia B cell acute (B-ALL) or a B-lymphoblastic leukemia-lymphoma (B-ALL). In some embodiments, the second gene is an RCSD1 gene, and the ABL1 fusion polypeptide: (a) comprises a fusion between an amino acid sequence encoded by exon 4, or a portion thereof, of an RCSD1 gene fused to an amino acid sequence encoded by exon 4, or a portion thereof, of an ABL1 gene; (b) results from a 5′ breakpoint within chromosomal coordinates chr1: 167654668-167654708 and/or a 3′ breakpoint within chromosomal coordinates chr9: 133738173-133738213; (c) comprises, in the N- to C-terminus direction, an amino acid sequence encoded by exons 1-3 and exon 4, or a portion thereof, of an RCSD1 gene, fused to an amino acid sequence encoded by exon 4, or a portion thereof, and exons 5-11 of an ABL1 gene; (d) is encoded by the nucleotide sequence of SEQ ID NO: 21, or a nucleotide sequence having at least about 70% homology thereto; and/or (e) comprises the amino acid sequence of SEQ ID NO: 42, or an amino acid sequence having at least about 70% homology thereto; optionally, wherein the cancer is a bone marrow leukemia, and optionally wherein the bone marrow leukemia is a bone marrow leukemia lymphocytic chronic (CLL). In some embodiments, the second gene is an SNX2 gene, and the ABL1 fusion polypeptide: (a) comprises a fusion between an amino acid sequence encoded by exon 3, or a portion thereof, of an SNX2 gene fused to an amino acid sequence encoded by exon 4, or a portion thereof, of an ABL1 gene; (b) results from: a 5′ breakpoint within chromosomal coordinates chr5: 122135451-122135491 and/or a 3′ breakpoint within chromosomal coordinates chr9: 133738222-133738262, or a 5′ breakpoint within chromosomal coordinates chr5: 122135445-122135485 and/or a 3′ breakpoint within chromosomal coordinates chr9: 133738214-133738254; and/or (c) comprises, in the N- to C-terminus direction, an amino acid sequence encoded by exons 1-2 and exon 3, or a portion thereof, of an SNX2 gene, fused to an amino acid sequence encoded by exon 4, or a portion thereof, and exons 5-11 of an ABL1 gene; optionally wherein the cancer is a leukemia, optionally wherein the leukemia is a bone marrow leukemia B cell acute (B-ALL) or a B-lymphoblastic leukemia-lymphoma (B-ALL).

In some embodiments, which may be combined with any of the preceding aspects or embodiments, the ABL1 fusion nucleic acid molecule comprises a fusion between an ABL1 gene, or a portion thereof, fused to a second gene, or a portion thereof, wherein the ABL1 fusion nucleic acid molecule comprises or results from an ABL1 breakpoint within exon 10 of ABL1, and wherein the ABL1 fusion nucleic acid molecule: (a) comprises, in 5′ to 3′ direction, exon 10, or a portion thereof, of an ABL1 gene fused to the second gene, or a portion thereof; (b) comprises a fusion between an exon or an intron, or a portion thereof, of the second gene fused to exon 10, or a portion thereof, of an ABL1 gene; (c) comprises or results from a breakpoint within chromosomal coordinates chr9: 133755953-133755993; and/or (d) comprises, in 5′ to 3′ direction, exons 1-9 and exon 10, or a portion thereof, of an ABL1 gene fused to one or more exons, or portions thereof, of the second gene; optionally wherein the cancer is soft tissue sarcoma, and optionally wherein the soft tissue sarcoma is not otherwise specified (NOS). In some embodiments, the ABL1 fusion polypeptide encoded by the ABL1 fusion nucleic acid molecule comprises a fusion between an ABL1 polypeptide, or a portion thereof, fused to a polypeptide encoded by a second gene, or a portion thereof, wherein the ABL1 fusion polypeptide results from an ABL1 breakpoint within exon 10 of an ABL1 gene, and wherein the ABL1 fusion polypeptide: (a) comprises, in the N- to C-terminus direction, an amino acid sequence encoded by exon 10, or a portion thereof, of an ABL1 gene fused to an amino acid sequence encoded by the second gene, or a portion thereof; (b) comprises a fusion between an amino acid sequence encoded by an exon, or a portion thereof, of the second gene fused to an amino acid sequence encoded by exon 10, or a portion thereof, of an ABL1 gene; (c) results from a breakpoint within chromosomal coordinates chr9: 133755953-133755993; and/or (d) comprises, in the N- to C-terminus direction, an amino acid sequence encoded by exons 1-9 and exon 10, or a portion thereof, of an ABL1 gene fused to an amino acid sequence encoded by one or more exons, or portions thereof, of the second gene; optionally wherein the cancer is soft tissue sarcoma, and optionally wherein the soft tissue sarcoma is not otherwise specified (NOS). In some embodiments, the second gene is a DNAH2 gene, and the ABL1 fusion nucleic acid molecule: (a) comprises a fusion between exon 10, or a portion thereof, of an ABL1 gene fused to exon 4, or a portion thereof, of a DNAH2 gene; (b) comprises or results from a 5′ breakpoint within chromosomal coordinates chr9: 133755953-133755993 and/or a 3′ breakpoint within chromosomal coordinates chr17: 7636399-7636439; and/or (c) comprises, in 5′ to 3′ direction, exons 1-9 and exon 10, or a portion thereof, of an ABL1 gene fused to exon 4, or a portion thereof, and exons 5-85 of a DNAH2 gene; optionally, wherein the cancer is a soft tissue sarcoma, and optionally wherein the soft tissue sarcoma is not otherwise specified (NOS). In some embodiments, the second gene is a DNAH2 gene, and the ABL1 fusion polypeptide: (a) comprises a fusion between an amino acid sequence encoded by exon 10, or a portion thereof, of an ABL1 gene fused to an amino acid sequence encoded by exon 4, or a portion thereof, of a DNAH2 gene; (b) results from a 5′ breakpoint within chromosomal coordinates chr9: 133755953-133755993 and/or a 3′ breakpoint within chromosomal coordinates chr17: 7636399-7636439; and/or (c) comprises, in the N- to C-terminus direction, an amino acid sequence encoded by exons 1-9 and exon 10, or a portion thereof, of an ABL1 gene fused to an amino acid sequence encoded by exon 4, or a portion thereof, and exons 5-85 of a DNAH2 gene; optionally, wherein the cancer is a soft tissue sarcoma, and optionally wherein the soft tissue sarcoma is not otherwise specified (NOS).

In some embodiments, which may be combined with any of the preceding aspects or embodiments, the ABL1 fusion nucleic acid molecule comprises a fusion between an ABL1 gene, or a portion thereof, fused to a second gene, or a portion thereof, wherein the ABL1 fusion nucleic acid molecule comprises or results from an ABL1 breakpoint within intron 4 of an ABL1 gene, and wherein the ABL1 fusion nucleic acid molecule: (a) comprises, in 5′ to 3′ direction, the second gene, or a portion thereof, fused to exon 5, or a portion thereof, of an ABL1 gene; (b) comprises a fusion between an exon or an intron of the second gene, or a portion thereof, fused to intron 4, or a portion thereof, of an ABL1 gene; (c) comprises or results from a breakpoint within chromosomal coordinates chr9: 133747364-133747762; and/or (d) comprises, in 5′ to 3′ direction, one or more exons, or portions thereof, of the second gene, fused to exon 5, or a portion thereof, and exons 6-11 of an ABL1 gene; optionally wherein the cancer is an ovary epithelial carcinoma, and optionally wherein the ovary epithelial carcinoma is not otherwise specified (NOS). In some embodiments, the ABL1 fusion polypeptide encoded by the ABL1 fusion nucleic acid molecule comprises a fusion between an ABL1 polypeptide, or a portion thereof, fused to a polypeptide encoded by a second gene, or a portion thereof, wherein the ABL1 fusion polypeptide results from an ABL1 breakpoint within intron 4 of an ABL1 gene, and wherein the ABL1 fusion polypeptide: (a) comprises, in the N- to C-terminus direction, an amino acid sequence encoded by the second gene, or a portion thereof, fused to an amino acid sequence encoded by exon 5, or a portion thereof, of an ABL1 gene; (b) comprises a fusion between an amino acid sequence encoded by an exon of the second gene, or a portion thereof, fused to an amino acid sequence encoded by exon 5, or a portion thereof, of an ABL1 gene; (c) results from a breakpoint within chromosomal coordinates chr9: 133747364-133747762; and/or (d) comprises, in the N- to C-terminus direction, an amino acid sequence encoded by one or more exons, or portions thereof, of the second gene, fused to an amino acid sequence encoded by exon 5, or a portion thereof, and exons 6-11 of an ABL1 gene; optionally wherein the cancer is an ovary epithelial carcinoma, and optionally wherein the ovary epithelial carcinoma is not otherwise specified (NOS). In some embodiments, the second gene is a MED27 gene, and the ABL1 fusion nucleic acid molecule: (a) comprises a fusion between exon 4, or a portion thereof, of a MED27 gene fused to exon 5, or a portion thereof, of an ABL1 gene; (b) comprises or results from a breakpoint within intron 4 of a MED27 gene and a breakpoint within intron 4 of an ABL1 gene; (c) comprises or results from a 5′ breakpoint within chromosomal coordinates chr9: 134774423-134776006 and/or a 3′ breakpoint within chromosomal coordinates chr9: 133747364-133747762; (d) comprises, in 5′ to 3′ direction, exons 1-3 and exon 4, or a portion thereof of a MED27 gene, fused to exon 5, or a portion thereof, and exons 6-11 of an ABL1gene; (e) comprises the nucleotide sequence of SEQ ID NO: 3, or a nucleotide sequence having at least about 70% homology thereto; and/or (f) encodes an ABL1 fusion polypeptide comprising the amino acid sequence of SEQ ID NO: 24, or an amino acid sequence having at least about 70% homology thereto; optionally, wherein the cancer is an ovary epithelial carcinoma, and optionally wherein the ovary epithelial carcinoma is not otherwise specified (NOS). In some embodiments, the second gene is a MED27 gene, and the ABL1 fusion polypeptide: (a) comprises a fusion between an amino acid sequence encoded by exon 4, or a portion thereof, of a MED27 gene fused to an amino acid sequence encoded by exon 5, or a portion thereof, of an ABL1 gene; (b) results from a breakpoint within intron 4 of a MED27 gene and a breakpoint within intron 4 of an ABL1 gene; (c) results from a 5′ breakpoint within chromosomal coordinates chr9: 134774423-134776006 and/or a 3′ breakpoint within chromosomal coordinates chr9: 133747364-133747762; (d) comprises, in the N- to C-terminus direction, an amino acid sequence encoded by exons 1-3 and exon 4, or a portion thereof, of a MED27 gene, fused to an amino acid sequence encoded by exon 5, or a portion thereof, and exons 6-11 of an ABL1 gene; (e) is encoded by the nucleotide sequence of SEQ ID NO: 3, or a nucleotide sequence having at least about 70% homology thereto; and/or (f) comprises the amino acid sequence of SEQ ID NO: 24, or an amino acid sequence having at least about 70% homology thereto; optionally, wherein the cancer is an ovary epithelial carcinoma, and optionally wherein the ovary epithelial carcinoma is not otherwise specified (NOS).

In some embodiments, which may be combined with any of the preceding aspects or embodiments, the ABL1 fusion nucleic acid molecule comprises a fusion between an ABL1 gene, or a portion thereof, fused to a second gene, or a portion thereof, wherein the ABL1 fusion nucleic acid molecule comprises or results from an ABL1 breakpoint within intron 5 of an ABL1 gene, and wherein the ABL1 fusion nucleic acid molecule: (a) comprises, in 5′ to 3′ direction, the second gene, or a portion thereof, fused to exon 6, or a portion thereof, of an ABL1 gene; (b) comprises a fusion between an exon or an intron, or a portion thereof, of the second gene fused to intron 5, or a portion thereof, of an ABL1 gene; (c) comprises or results from a breakpoint within chromosomal coordinates chr9: 133748211-133748501; and/or (d) comprises, in 5′ to 3′ direction, one or more exons, or portions thereof, of the second gene, fused to exon 6, or a portion thereof, and exons 7-11 of an ABL1 gene; optionally wherein the cancer is a testis germ cell tumor, and optionally wherein the testis germ cell tumor is a mixed testis germ cell tumor. In some embodiments, the ABL1 fusion polypeptide encoded by the ABL1 fusion nucleic acid molecule comprises a fusion between an ABL1 polypeptide, or a portion thereof, fused to a polypeptide encoded by a second gene, or a portion thereof, wherein the ABL1 fusion polypeptide results from an ABL1 breakpoint within intron 5 of ABL1, and wherein the ABL1 fusion polypeptide: (a) comprises, in the N- to C-terminus direction, an amino acid sequence encoded by the second gene, or a portion thereof, fused to an amino acid sequence encoded by exon 6, or a portion thereof, of an ABL1 gene; (b) comprises a fusion between an amino acid sequence encoded by an exon, or a portion thereof, of the second gene fused to an amino acid sequence encoded by exon 6, or a portion thereof, of an ABL1 gene; (c) results from a breakpoint within chromosomal coordinates chr9: 133748211-133748501; (d) comprises, in the N- to C-terminus direction, an amino acid sequence encoded by one or more exons, or portions thereof, of the second gene, fused to an amino acid sequence encoded by exon 6, or a portion thereof, and exons 7-11 of an ABL1 gene; optionally wherein the cancer is a testis germ cell tumor, and optionally wherein the testis germ cell tumor is a mixed testis germ cell tumor. In some embodiments, the second gene is an SLC27A4 gene, and the ABL1 fusion nucleic acid molecule: (a) comprises a fusion between exon 12, or a portion thereof, of a SLC27A4 gene fused to exon 6, or a portion thereof, of an ABL1 gene; (b) comprises or results from a breakpoint within intron 12 of a SLC27A4 gene and a breakpoint within intron 5 of an ABL1 gene; (c) comprises or results from a 5′ breakpoint within chromosomal coordinates chr9: 131119603-131119801 and/or a 3′ breakpoint within chromosomal coordinates chr9: 133748211-133748501; (d) comprises, in 5′ to 3′ direction, exons 1-11 and exon 12, or a portion thereof, of an SLC27A4 gene, fused to exon 6, or a portion thereof, and exons 7-11 of an ABL1 gene; (e) comprises the nucleotide sequence of SEQ ID NO: 5, or a nucleotide sequence having at least about 70% homology thereto; and/or (f) encodes an ABL1 fusion polypeptide comprising the amino acid sequence of SEQ ID NO: 26, or an amino acid sequence having at least about 70% homology thereto; optionally, wherein the cancer is a testis germ cell tumor, wherein the testis germ cell tumor is a mixed testis germ cell tumor. In some embodiments, the second gene is an SLC27A4 gene, and the ABL1 fusion polypeptide: (a) comprises a fusion between an amino acid sequence encoded by exon 12, or a portion thereof, of a SLC27A4 gene fused to an amino acid sequence encoded by exon 6, or a portion thereof, of an ABL1 gene; (b) results from a breakpoint within intron 12 of a SLC27A4 gene and a breakpoint within intron 5 of an ABL1 gene; (c) results from a 5′ breakpoint within chromosomal coordinates chr9: 131119603-131119801 and/or a 3′ breakpoint within chromosomal coordinates chr9: 133748211-133748501; (d) comprises, in the N- to C-terminus direction, an amino acid sequence encoded by exons 1-11 and exon 12, or a portion thereof, of an SLC27A4 gene, fused to an amino acid sequence encoded by exon 6, or a portion thereof, and exons 7-11 of an ABL1 gene; (e) is encoded by the nucleotide sequence of SEQ ID NO: 5, or a nucleotide sequence having at least about 70% homology thereto; and/or (f) comprises the amino acid sequence of SEQ ID NO: 26, or an amino acid sequence having at least about 70% homology thereto; optionally, wherein the cancer is a testis germ cell tumor, wherein the testis germ cell tumor is a mixed testis germ cell tumor.

In some embodiments, which may be combined with any of the preceding aspects or embodiments, the ABL1 fusion nucleic acid molecule comprises a fusion between an ABL1 gene, or a portion thereof, fused to a BCR gene, or a portion thereof, wherein the cancer is lung adenocarcinoma, and wherein the ABL1 fusion nucleic acid molecule: (a) comprises, in 5′ to 3′ direction, a BCR gene, or a portion thereof, fused to an ABL1 gene, or a portion thereof; (b) comprises: a fusion between exon 12, or a portion thereof, of a BCR gene fused to exon 2, or a portion thereof, of an ABL1 gene, a fusion between exon 13, or a portion thereof, of a BCR gene fused to exon 2, or a portion thereof, of an ABL1 gene, or a fusion between exon 14, or a portion thereof, of a BCR gene fused to exon 2, or a portion thereof, of an ABL1 gene; (c) comprises or results from a breakpoint within intron 12, intron 13 or intron 14 of a BCR gene, and/or a breakpoint within intron 1 of an ABL1 gene; (d) comprises or results from: a 5′ breakpoint within chromosomal coordinates chr22: 23632033-23632321 and/or a 3′ breakpoint within chromosomal coordinates chr9: 133591666-133591902, a 5′ breakpoint within chromosomal coordinates chr22: 23634537-23634672 and/or a 3′ breakpoint within chromosomal coordinates chr9: 133672815-133673059, a 5′ breakpoint within chromosomal coordinates chr22: 23634041-23634323 and/or a 3′ breakpoint within chromosomal coordinates chr9: 133708182-133708567, a 5′ breakpoint within chromosomal coordinates chr22: 23632683-23632948 and/or a 3′ breakpoint within chromosomal coordinates chr9: 133613427-133613626, or a 5′ breakpoint within chromosomal coordinates chr22: 23631693-23632188 and/or a 3′ breakpoint within chromosomal coordinates chr9: 133607683-133608245; (e) comprises, in 5′ to 3′ direction: exons 1-11 and exon 12, or a portion thereof, of a BCR gene fused to exon 2, or a portion thereof, and exons 3-11 of an ABL1 gene, exons 1-12 and exon 13, or a portion thereof, of a BCR gene fused to exon 2, or a portion thereof, and exons 3-11 of an ABL1 gene, or exons 1-13 and exon 14, or a portion thereof, of a BCR gene, fused to exon 2, or a portion thereof, and exons 3-11 of an ABL1 gene; (f) comprises the nucleotide sequence of any of SEQ ID NOs: 6-10, or a nucleotide sequence having at least about 70% homology thereto; and/or (g) encodes an ABL1 fusion polypeptide comprising the amino acid sequence of any of SEQ ID NOs: 27-31, or an amino acid sequence having at least about 70% homology thereto. In some embodiments, the ABL1 fusion polypeptide encoded by the ABL1 fusion nucleic acid molecule comprises a fusion between an ABL1 polypeptide, or a portion thereof, fused to a BCR polypeptide, or a portion thereof, wherein the cancer is lung adenocarcinoma, and wherein the ABL1 fusion polypeptide: (a) comprises, in the N- to C-terminus direction, a BCR polypeptide, or a portion thereof, fused to an ABL1 polypeptide, or a portion thereof; (b) comprises: a fusion between an amino acid sequence encoded by exon 12, or a portion thereof, of a BCR gene fused to an amino acid sequence encoded by exon 2, or a portion thereof, of an ABL1 gene, a fusion between an amino acid sequence encoded by exon 13, or a portion thereof, of a BCR gene fused to an amino acid sequence encoded by exon 2, or a portion thereof, of an ABL1 gene, or a fusion between an amino acid sequence encoded by exon 14, or a portion thereof, of a BCR gene fused to an amino acid sequence encoded by exon 2, or a portion thereof, of an ABL1 gene; (c) results from a breakpoint within intron 12, intron 13 or intron 14 of a BCR gene, and/or a breakpoint within intron 1 of an ABL1 gene; (d) results from: a 5′ breakpoint within chromosomal coordinates chr22: 23632033-23632321 and/or a 3′ breakpoint within chromosomal coordinates chr9: 133591666-133591902, a 5′ breakpoint within chromosomal coordinates chr22: 23634537-23634672 and/or a 3′ breakpoint within chromosomal coordinates chr9: 133672815-133673059, a 5′ breakpoint within chromosomal coordinates chr22: 23634041-23634323 and/or a 3′ breakpoint within chromosomal coordinates chr9: 133708182-133708567, a 5′ breakpoint within chromosomal coordinates chr22: 23632683-23632948 and/or a 3′ breakpoint within chromosomal coordinates chr9: 133613427-133613626, or a 5′ breakpoint within chromosomal coordinates chr22: 23631693-23632188 and/or a 3′ breakpoint within chromosomal coordinates chr9: 133607683-133608245; (e) comprises, in the N- to C-terminus direction: an amino acid sequence encoded by exons 1-11 and exon 12, or a portion thereof, of a BCR gene fused to an amino acid sequence encoded by exon 2, or a portion thereof, and exons 3-11 of an ABL1 gene, an amino acid sequence encoded by exons 1-12 and exon 13, or a portion thereof, of a BCR gene fused to an amino acid sequence encoded by exon 2, or a portion thereof, and exons 3-11 of an ABL1 gene, or an amino acid sequence encoded by exons 1-13 and exon 14, or a portion thereof, of a BCR gene, fused to an amino acid sequence encoded by exon 2, or a portion thereof, and exons 3-11 of an ABL1 gene; (f) is encoded by the nucleotide sequence of any of SEQ ID NOs: 6-10, or a nucleotide sequence having at least about 70% homology thereto; and/or (g) comprises the amino acid sequence of any of SEQ ID NOs: 27-31, or an amino acid sequence having at least about 70% homology thereto.

In some embodiments, which may be combined with any of the preceding aspects or embodiments, the ABL1 fusion nucleic acid molecule comprises a fusion between an ABL1 gene, or a portion thereof, fused to a BCR gene, or a portion thereof, wherein the cancer is head and neck squamous cell carcinoma (HNSCC), and wherein the ABL1 fusion nucleic acid molecule: (a) comprises, in 5′ to 3′ direction, a BCR gene, or a portion thereof, fused to an ABL1 gene, or a portion thereof; (b) comprises a fusion between exon 13 or a portion thereof, or exon 14 or a portion thereof, of a BCR gene fused to exon 2, or a portion thereof, of an ABL1 gene; (c) comprises or results from a breakpoint within intron 13 or intron 14 of a BCR gene and/or a breakpoint within intron 1 of an ABL1 gene; (d) comprises or results from: a 5′ breakpoint within chromosomal coordinates chr22: 23634011-23634460 and/or a 3′ breakpoint within chromosomal coordinates chr9: 133645693-133646017, a 5′ breakpoint within chromosomal coordinates chr22: 23634211-23634632 and/or a 3′ breakpoint within chromosomal coordinates chr9: 133619101-133619395, a 5′ breakpoint within chromosomal coordinates chr22: 23631687-23632492 and/or a 3′ breakpoint within chromosomal coordinates chr9: 133604425-133604577, or a 5′ breakpoint within chromosomal coordinates chr22: 23632288-23632656 and/or a 3′ breakpoint within chromosomal coordinates chr9: 133663291-133663682; (e) comprises, in 5′ to 3′ direction: exons 1-13 and exon 14, or a portion thereof, of a BCR gene, fused to exon 2, or a portion thereof, and exons 3-11 of an ABL1 gene, or exons 1-12 and exon 13, or a portion thereof, of a BCR gene, fused to exon 2, or a portion thereof, and exons 3-11 of ABL1; (f) comprises the nucleotide sequence of any of SEQ ID NOs: 11-12, or a nucleotide sequence having at least about 70% homology thereto; and/or (g) encodes an ABL1 fusion polypeptide comprising the amino acid sequence of any of SEQ ID NOs: 32-33, or an amino acid sequence having at least about 70% homology thereto. In some embodiments, the ABL1 fusion polypeptide encoded by the ABL1 fusion nucleic acid molecule comprises a fusion between an ABL1 polypeptide, or a portion thereof, fused to a BCR polypeptide, or a portion thereof, wherein the cancer is head and neck squamous cell carcinoma (HNSCC), and wherein the ABL1 fusion polypeptide: (a) comprises, in the N- to C-terminus direction, a BCR polypeptide, or a portion thereof, fused to an ABL1 polypeptide, or a portion thereof; (b) comprises a fusion between an amino acid sequence encoded by exon 13 or a portion thereof, or exon 14 or a portion thereof, of a BCR gene fused to an amino acid sequence encoded by exon 2, or a portion thereof, of an ABL1 gene; (c) results from a breakpoint within intron 13 or intron 14 of a BCR gene and/or a breakpoint within intron 1 of an ABL1 gene; (d) results from: a 5′ breakpoint within chromosomal coordinates chr22: 23634011-23634460 and/or a 3′ breakpoint within chromosomal coordinates chr9: 133645693-133646017, a 5′ breakpoint within chromosomal coordinates chr22: 23634211-23634632 and/or a 3′ breakpoint within chromosomal coordinates chr9: 133619101-133619395, a 5′ breakpoint within chromosomal coordinates chr22: 23631687-23632492 and/or a 3′ breakpoint within chromosomal coordinates chr9: 133604425-133604577, or a 5′ breakpoint within chromosomal coordinates chr22: 23632288-23632656 and/or a 3′ breakpoint within chromosomal coordinates chr9: 133663291-133663682; (e) comprises, in the N- to C-terminus direction: an amino acid sequence encoded by exons 1-13 and exon 14, or a portion thereof, of a BCR gene, fused to an amino acid sequence encoded by exon 2, or a portion thereof, and exons 3-11 of an ABL1 gene, or an amino acid sequence encoded by exons 1-12 and exon 13, or a portion thereof, of a BCR gene, fused to an amino acid sequence encoded by exon 2, or a portion thereof, and exons 3-11 of ABL1; (f) is encoded by the nucleotide sequence of any of SEQ ID NOs: 11-12, or a nucleotide sequence having at least about 70% homology thereto; and/or (g) comprises the amino acid sequence of any of SEQ ID NOs: 32-33, or an amino acid sequence having at least about 70% homology thereto.

In some embodiments, which may be combined with any of the preceding aspects or embodiments, the ABL1 fusion nucleic acid molecule comprises a fusion between an ABL1 gene, or a portion thereof, fused to a BCR gene, or a portion thereof, wherein the cancer is bone sarcoma, and wherein the ABL1 fusion nucleic acid molecule: (a) comprises, in 5′ to 3′ direction, a BCR gene, or a portion thereof, fused to an ABL1 gene, or a portion thereof; (b) comprises a fusion between exon 13, or a portion thereof, of a BCR gene fused to exon 2, or a portion thereof, of an ABL1 gene; (c) comprises or results from a breakpoint within intron 13 of a BCR gene and/or a breakpoint within intron 1 of an ABL1 gene; (d) comprises or results from a 5′ breakpoint within chromosomal coordinates chr22: 23631722-23631762 and/or a 3′ breakpoint within chromosomal coordinates chr9: 133729484-133729524; (e) comprises, in 5′ to 3′ direction, exons 1-12 and exon 13, or a portion thereof, of a BCR gene fused to exon 2, or a portion thereof, and exons 3-11 of an ABL1 gene; (f) comprises the nucleotide sequence of SEQ ID NO: 13, or a nucleotide sequence having at least about 70% homology thereto; and/or (g) encodes an ABL1 fusion polypeptide comprising the amino acid sequence of SEQ ID NO: 34, or an amino acid sequence having at least about 70% homology thereto; optionally wherein the bone sarcoma is not otherwise specified (NOS). In some embodiments, the ABL1 fusion polypeptide encoded by the ABL1 fusion nucleic acid molecule comprises a fusion between an ABL1 polypeptide, or a portion thereof, fused to a BCR polypeptide, or a portion thereof, wherein the cancer is bone sarcoma, and wherein the ABL1 fusion polypeptide: (a) comprises, in the N- to C-terminus direction, a BCR polypeptide, or a portion thereof, fused to an ABL1 polypeptide, or a portion thereof; (b) comprises a fusion between an amino acid sequence encoded by exon 13, or a portion thereof, of a BCR gene fused to an amino acid sequence encoded by exon 2, or a portion thereof, of an ABL1 gene; (c) results from a breakpoint within intron 13 of a BCR gene and/or a breakpoint within intron 1 of an ABL1 gene; (d) results from a 5′ breakpoint within chromosomal coordinates chr22: 23631722-23631762 and/or a 3′ breakpoint within chromosomal coordinates chr9: 133729484-133729524; (e) comprises, in the N- to C-terminus direction, an amino acid sequence encoded by exons 1-12 and exon 13, or a portion thereof, of a BCR gene, fused to an amino acid sequence encoded by exon 2, or a portion thereof, and exons 3-11 of an ABL1 gene; (f) is encoded by the nucleotide sequence of SEQ ID NO: 13, or a nucleotide sequence having at least about 70% homology thereto; and/or (g) comprises the amino acid sequence of SEQ ID NO: 34, or an amino acid sequence having at least about 70% homology thereto; optionally wherein the bone sarcoma is not otherwise specified (NOS).

In some embodiments, which may be combined with any of the preceding aspects or embodiments, the ABL1 fusion nucleic acid molecule comprises a fusion between an ABL1 gene, or a portion thereof, fused to a BCR gene, or a portion thereof, wherein the cancer is esophagus adenocarcinoma, and wherein the ABL1 fusion nucleic acid molecule: (a) comprises, in 5′ to 3′ direction a BCR gene, or a portion thereof, fused to an ABL1 gene, or a portion thereof; (b) comprises a fusion between exon 14, or a portion thereof, of a BCR gene fused to exon 2, or a portion thereof, of an ABL1 gene; (c) comprises or results from a breakpoint within intron 14 of a BCR gene and/or a breakpoint within intron 1 of an ABL1 gene; (d) comprises or results from a 5′ breakpoint within chromosomal coordinates chr22: 23633346-23634163 and/or a 3′ breakpoint within chromosomal coordinates chr9: 133609201-133609909; (e) comprises, in 5′ to 3′ direction, exons 1-13 and exon 14, or a portion thereof, of a BCR gene, fused to exon 2, or a portion thereof, and exons 3-11 of an ABL1 gene; (f) comprises the nucleotide sequence of SEQ ID NO: 14, or a nucleotide sequence having at least about 70% homology thereto; and/or (g) encodes an ABL1 fusion polypeptide comprising the amino acid sequence of SEQ ID NO: 35, or an amino acid sequence having at least about 70% homology thereto. In some embodiments, the ABL1 fusion polypeptide encoded by the ABL1 fusion nucleic acid molecule comprises a fusion between an ABL1 polypeptide, or a portion thereof, fused to a BCR polypeptide, or a portion thereof, wherein the cancer is esophagus adenocarcinoma, and wherein the ABL1 fusion polypeptide: (a) comprises, in the N- to C-terminus direction, a BCR polypeptide, or a portion thereof, fused to an ABL1 polypeptide, or a portion thereof; (b) comprises a fusion between an amino acid sequence encoded by exon 14, or a portion thereof, of a BCR gene fused to an amino acid sequence encoded by exon 2, or a portion thereof, of an ABL1 gene; (c) results from a breakpoint within intron 14 of a BCR gene and/or a breakpoint within intron 1 of an ABL1 gene; (d) results from a 5′ breakpoint within chromosomal coordinates chr22: 23633346-23634163 and/or a 3′ breakpoint within chromosomal coordinates chr9: 133609201-133609909; (e) comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; (f) is encoded by the nucleotide sequence of SEQ ID NO: 14, or a nucleotide sequence having at least about 70% homology thereto; and/or (g) comprises the amino acid sequence of SEQ ID NO: 35, or an amino acid sequence having at least about 70% homology thereto.

In some embodiments, which may be combined with any of the preceding aspects or embodiments, the ABL1 fusion nucleic acid molecule comprises a fusion between an ABL1 gene, or a portion thereof, fused to a BCR gene, or a portion thereof, wherein the cancer is gastroesophageal junction adenocarcinoma, and wherein the ABL1 fusion nucleic acid molecule: (a) comprises, in the 5′ to 3′ direction, a BCR gene, or a portion thereof, fused to an ABL1 gene, or a portion thereof; (b) comprises a fusion between exon 14, or a portion thereof, of a BCR gene fused to exon 2, or a portion thereof, of an ABL1 gene; (c) comprises or results from a breakpoint within intron 14 of a BCR gene and/or a breakpoint within intron 1 of an ABL1 gene; (d) comprises or results from a 5′ breakpoint within chromosomal coordinates chr22: 23633432-23633796 and/or a 3′ breakpoint within chromosomal coordinates chr9: 133623903-133624461; (e) comprises, in 5′ to 3′ direction, exons 1-13 and exon 14, or a portion thereof, of a BCR gene fused to exon 2, or a portion thereof, and exons 3-11 of an ABL1 gene; (f) comprises the nucleotide sequence of SEQ ID NO: 15, or a nucleotide sequence having at least about 70% homology thereto; and/or (g) encodes an ABL1 fusion polypeptide comprising the amino acid sequence of SEQ ID NO: 36, or an amino acid sequence having at least about 70% homology thereto. In some embodiments, the ABL1 fusion polypeptide encoded by the ABL1 fusion nucleic acid molecule comprises a fusion between an ABL1 polypeptide, or a portion thereof, fused to a BCR polypeptide, or a portion thereof, wherein the cancer is gastroesophageal junction adenocarcinoma, and wherein the ABL1 fusion polypeptide: (a) comprises, in the N- to C-terminus direction, a BCR polypeptide, or a portion thereof, fused to an ABL1 polypeptide, or a portion thereof; (b) comprises a fusion between an amino acid sequence encoded by exon 14, or a portion thereof, of a BCR gene fused to an amino acid sequence encoded by exon 2, or a portion thereof, of an ABL1 gene; (c) results from a breakpoint within intron 14 of a BCR gene and/or a breakpoint within intron 1 of an ABL1 gene; (d) results from a 5′ breakpoint within chromosomal coordinates chr22: 23633432-23633796 and/or a 3′ breakpoint within chromosomal coordinates chr9: 133623903-133624461; (e) comprises, in the N- to C-terminus direction, an amino acid sequence encoded by exons 1-13 and exon 14, or a portion thereof, of a BCR gene fused to an amino acid sequence encoded by exon 2, or a portion thereof, and exons 3-11 of an ABL1 gene; (f) is encoded by the nucleotide sequence of SEQ ID NO: 15, or a nucleotide sequence having at least about 70% homology thereto; and/or (g) comprises the amino acid sequence of SEQ ID NO: 36, or an amino acid sequence having at least about 70% homology thereto.

In some embodiments, which may be combined with any of the preceding aspects or embodiments, the ABL1 fusion nucleic acid molecule comprises a fusion between an ABL1 gene, or a portion thereof, fused to a BCR gene, or a portion thereof, wherein the cancer is liver cholangiocarcinoma, and wherein the ABL1 fusion nucleic acid molecule: (a) comprises, in 5′ to 3′ direction, a BCR gene, or a portion thereof, fused to an ABL1 gene, or a portion thereof; (b) comprises a fusion between exon 14, or a portion thereof, of a BCR gene fused to exon 2, or a portion thereof, of an ABL1 gene; (c) comprises or results from a breakpoint within intron 14 of a BCR gene and/or a breakpoint within intron 1 of an ABL1 gene; (d) comprises or results from a 5′ breakpoint within chromosomal coordinates chr22: 23633837-23634208 and/or a 3′ breakpoint within chromosomal coordinates chr9: 133665426-133665727; (e) comprises, in 5′ to 3′ direction, exons 1-13 and exon 14, or a portion thereof, of a BCR gene, fused to exon 2, or a portion thereof, and exons 3-11 of an ABL1 gene; (f) comprises the nucleotide sequence of SEQ ID NO: 16, or a nucleotide sequence having at least about 70% homology thereto; and/or (g) encodes an ABL1 fusion polypeptide comprising the amino acid sequence of SEQ ID NO: 37, or an amino acid sequence having at least about 70% homology thereto. In some embodiments, the ABL1 fusion polypeptide encoded by the ABL1 fusion nucleic acid molecule comprises a fusion between an ABL1 polypeptide, or a portion thereof, fused to a BCR polypeptide, or a portion thereof, wherein the cancer is liver cholangiocarcinoma, and wherein the ABL1 fusion polypeptide: (a) comprises, in the N- to C-terminus direction, a BCR polypeptide, or a portion thereof, fused to an ABL1 polypeptide, or a portion thereof; (b) comprises a fusion between an amino acid sequence encoded by exon 14, or a portion thereof, of a BCR gene fused to an amino acid sequence encoded by exon 2, or a portion thereof, of an ABL1 gene; (c) results from a breakpoint within intron 14 of a BCR gene and/or a breakpoint within intron 1 of an ABL1 gene; (d) results from a 5′ breakpoint within chromosomal coordinates chr22: 23633837-23634208 and/or a 3′ breakpoint within chromosomal coordinates chr9: 133665426-133665727; (e) comprises, in the N- to C-terminus direction, an amino acid sequence encoded by exons 1-13 and exon 14, or a portion thereof, of BCR, fused to an amino acid sequence encoded by exon 2, or a portion thereof, and exons 3-11 of ABL1; (f) is encoded by the nucleotide sequence of SEQ ID NO: 16, or a nucleotide sequence having at least about 70% homology thereto; and/or (g) comprises the amino acid sequence of SEQ ID NO: 37, or an amino acid sequence having at least about 70% homology thereto.

In some embodiments, which may be combined with any of the preceding aspects or embodiments, the ABL1 fusion nucleic acid molecule comprises a fusion between an ABL1 gene, or a portion thereof, fused to a BCR gene, or a portion thereof, wherein the cancer is pancreas ductal adenocarcinoma, and wherein the ABL1 fusion nucleic acid molecule: (a) comprises, in 5′ to 3′ direction a BCR gene, or a portion thereof, fused to an ABL1 gene, or a portion thereof; (b) comprises a fusion between exon 14, or a portion thereof, of a BCR gene fused to exon 2, or a portion thereof, of an ABL1 gene; (c) comprises or results from a breakpoint within intron 14 of a BCR gene and/or a breakpoint within intron 1 of an ABL1 gene; (d) comprises or results from a 5′ breakpoint within chromosomal coordinates chr22: 23632858-23633068 and/or a 3′ breakpoint within chromosomal coordinates chr9: 133701595-133701846; (e) comprises, in 5′ to 3′ direction, exons 1-13 and exon 14, or a portion thereof, of a BCR gene, fused to exon 2, or a portion thereof, and exons 3-11 of an ABL1 gene; (f) comprises the nucleotide sequence of SEQ ID NO: 17, or a nucleotide sequence having at least about 70% homology thereto; and/or (g) encodes an ABL1 fusion polypeptide comprising the amino acid sequence of SEQ ID NO: 38, or an amino acid sequence having at least about 70% homology thereto. In some embodiments, the ABL1 fusion polypeptide encoded by the ABL1 fusion nucleic acid molecule comprises a fusion between an ABL1 polypeptide, or a portion thereof, fused to a BCR polypeptide, or a portion thereof, wherein the cancer is pancreas ductal adenocarcinoma, and wherein the ABL1 fusion polypeptide: (a) comprises, in the N- to C-terminus direction, a BCR polypeptide, or a portion thereof, fused to an ABL1 polypeptide, or a portion thereof; (b) comprises a fusion between an amino acid sequence encoded by exon 14, or a portion thereof, of a BCR gene fused to an amino acid sequence encoded by exon 2, or a portion thereof, of an ABL1 gene; (c) results from a breakpoint within intron 14 of a BCR gene and/or a breakpoint within intron 1 of an ABL1 gene; (d) results from a 5′ breakpoint within chromosomal coordinates chr22: 23632858-23633068 and/or a 3′ breakpoint within chromosomal coordinates chr9: 133701595-133701846; (e) comprises, in the N- to C-terminus direction, an amino acid sequence encoded by exons 1-13 and exon 14, or a portion thereof, of a BCR gene fused to an amino acid sequence encoded by exon 2, or a portion thereof, and exons 3-11 of an ABL1 gene; (f) is encoded by the nucleotide sequence of SEQ ID NO: 17, or a nucleotide sequence having at least about 70% homology thereto; and/or (g) comprises the amino acid sequence of SEQ ID NO: 38, or an amino acid sequence having at least about 70% homology thereto.

In some embodiments, which may be combined with any of the preceding aspects or embodiments, the ABL1 fusion nucleic acid molecule comprises a fusion between an ABL1 gene, or a portion thereof, fused to a BCR gene, or a portion thereof, wherein the cancer is ovary serous carcinoma, and wherein the ABL1 fusion nucleic acid molecule: (a) comprises, in 5′ to 3′ direction a BCR gene, or a portion thereof, fused to an ABL1 gene, or a portion thereof; (b) comprises a fusion between exon 14, or a portion thereof, of a BCR gene fused to exon 2, or a portion thereof, of an ABL1 gene; (c) comprises or results from a breakpoint within intron 14 of a BCR gene and/or a breakpoint within intron 1 of an ABL1 gene; (d) comprises or results from a 5′ breakpoint within chromosomal coordinates chr22: 23633208-23633695 and/or a 3′ breakpoint within chromosomal coordinates chr9: 133702617-133702992; (e) comprises, in 5′ to 3′ direction, exons 1-13 and exon 14, or a portion thereof, of a BCR gene, fused to exon 2, or a portion thereof, and exons 3-11 of an ABL1 gene; (f) comprises the nucleotide sequence of SEQ ID NO: 18, or a nucleotide sequence having at least about 70% homology thereto; and/or (g) encodes an ABL1 fusion polypeptide comprising the amino acid sequence of SEQ ID NO: 39, or an amino acid sequence having at least about 70% homology thereto. In some embodiments, the ABL1 fusion polypeptide encoded by the ABL1 fusion nucleic acid molecule comprises a fusion between an ABL1 polypeptide, or a portion thereof, fused to a BCR polypeptide, or a portion thereof, wherein the cancer is ovary serous carcinoma, and wherein the ABL1 fusion polypeptide: (a) comprises, in the N- to C-terminus direction, a BCR polypeptide, or a portion thereof, fused to an ABL1 polypeptide, or a portion thereof; (b) comprises a fusion between an amino acid sequence encoded by exon 14, or a portion thereof, of a BCR gene fused to an amino acid sequence encoded by exon 2, or a portion thereof, of an ABL1 gene; (c) results from a breakpoint within intron 14 of a BCR gene and/or a breakpoint within intron 1 of an ABL1 gene; (d) results from a 5′ breakpoint within chromosomal coordinates chr22: 23633208-23633695 and/or a 3′ breakpoint within chromosomal coordinates chr9: 133702617-133702992; (e) comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; (f) is encoded by the nucleotide sequence of SEQ ID NO: 18, or a nucleotide sequence having at least about 70% homology thereto; and/or (g) comprises the amino acid sequence of SEQ ID NO: 39, or an amino acid sequence having at least about 70% homology thereto.

In some embodiments, which may be combined with any of the preceding aspects or embodiments, the ABL1 fusion nucleic acid molecule comprises a fusion between an ABL1 gene, or a portion thereof, fused to a BCR gene, or a portion thereof, wherein the cancer is lung squamous cell carcinoma (SCC), and wherein the ABL1 fusion nucleic acid molecule: (a) comprises, in 5′ to 3′ direction a BCR gene, or a portion thereof, fused to an ABL1 gene, or a portion thereof; (b) comprises a fusion between exon 14, or a portion thereof, of a BCR gene fused to exon 2, or a portion thereof, of an ABL1 gene; (c) comprises or results from a breakpoint within intron 14 of a BCR gene and/or a breakpoint within intron 1 of an ABL1 gene; (d) comprises or results from: a 5′ breakpoint within chromosomal coordinates chr22: 23634171-23634647 and/or a 3′ breakpoint within chromosomal coordinates chr9: 133638886-133639512, or a 5′ breakpoint within chromosomal coordinates chr22: 23633910-23634678 and/or a 3′ breakpoint within chromosomal coordinates chr9: 133724778-133725351; and/or (e) comprises, in 5′ to 3′ direction, exons 1-13 and exon 14, or a portion thereof, of a BCR gene, fused to exon 2, or a portion thereof, and exons 3-11 of an ABL1 gene. In some embodiments, the ABL1 fusion polypeptide encoded by the ABL1 fusion nucleic acid molecule comprises a fusion between an ABL1 polypeptide, or a portion thereof, fused to a BCR polypeptide, or a portion thereof, wherein the cancer is lung squamous cell carcinoma (SCC), and wherein the ABL1 fusion polypeptide: (a) comprises, in the N- to C-terminus direction, a BCR polypeptide, or a portion thereof, fused to an ABL1 polypeptide, or a portion thereof; (b) comprises a fusion between an amino acid sequence encoded by exon 14, or a portion thereof, of a BCR gene fused to an amino acid sequence encoded by exon 2, or a portion thereof, of an ABL1 gene; (c) results from a breakpoint within intron 14 of a BCR gene and/or a breakpoint within intron 1 of an ABL1 gene; (d) results from: a 5′ breakpoint within chromosomal coordinates chr22: 23634171-23634647 and/or a 3′ breakpoint within chromosomal coordinates chr9: 133638886-133639512, or a 5′ breakpoint within chromosomal coordinates chr22: 23633910-23634678 and/or a 3′ breakpoint within chromosomal coordinates chr9: 133724778-133725351; and/or (e) comprises, in the N- to C-terminus direction, an amino acid sequence encoded by exons 1-13 and exon 14, or a portion thereof, of a BCR gene fused to an amino acid sequence encoded by exon 2, or a portion thereof, and exons 3-11 of an ABL1 gene.

In some embodiments, which may be combined with any of the preceding aspects or embodiments, the ABL1 fusion nucleic acid molecule comprises a fusion between an ABL1 gene, or a portion thereof, fused to a NUP214 gene, or a portion thereof, wherein the cancer is bone marrow leukemia lymphocytic chronic (CLL), and wherein the ABL1 fusion nucleic acid molecule: (a) comprises, in 5′ to 3′ direction, a NUP214 gene, or a portion thereof, fused to an ABL1 gene, or a portion thereof; (b) comprises a fusion between exon 34, or a portion thereof, of a NUP214 gene fused to exon 3, or a portion thereof, of an ABL1 gene; (c) comprises or results from a 5′ breakpoint within chromosomal coordinates chr9: 134106039-134106079 and/or a 3′ breakpoint within chromosomal coordinates chr9: 133730222-133730262; (d) comprises, in 5′ to 3′ direction, exons 1-33 and exon 34, or a portion thereof, of a NUP214 gene, fused to exon 3, or a portion thereof, and exons 4-11 of an ABL1 gene; (e) comprises the nucleotide sequence of SEQ ID NO: 19, or a nucleotide sequence having at least about 70% homology thereto; and/or (f) encodes an ABL1 fusion polypeptide comprising the amino acid sequence of SEQ ID NO: 40, or an amino acid sequence having at least about 70% homology thereto. In some embodiments, the ABL1 fusion polypeptide encoded by the ABL1 fusion nucleic acid molecule comprises a fusion between an ABL1 polypeptide, or a portion thereof, fused to a NUP214 polypeptide, or a portion thereof, wherein the cancer is bone marrow leukemia lymphocytic chronic (CLL), and wherein the ABL1 fusion polypeptide: (a) comprises, in the N- to C-terminus direction, a NUP214 polypeptide, or a portion thereof, fused to an ABL1 polypeptide, or a portion thereof; (b) comprises a fusion between an amino acid sequence encoded by exon 34, or a portion thereof, of a NUP214 gene fused to an amino acid sequence encoded by exon 3, or a portion thereof, of an ABL1 gene; (c) results from a 5′ breakpoint within chromosomal coordinates chr9: 134106039-134106079 and/or a 3′ breakpoint within chromosomal coordinates chr9: 133730222-133730262; (d) comprises, in the N- to C-terminus direction, an amino acid sequence encoded by exons 1-33 and exon 34, or a portion thereof, of a NUP214 gene fused to an amino acid sequence encoded by exon 3, or a portion thereof, and exons 4-11 of an ABL1 gene; (e) is encoded by the nucleotide sequence of SEQ ID NO: 19, or a nucleotide sequence having at least about 70% homology thereto; and/or (f) comprises the amino acid sequence of SEQ ID NO: 40, or an amino acid sequence having at least about 70% homology thereto.

In some embodiments, which may be combined with any of the preceding aspects or embodiments, the ABL1 fusion nucleic acid molecule comprises a fusion between an ABL1 gene, or a portion thereof, fused to a NUP214 gene, or a portion thereof, wherein the cancer is soft tissue liposarcoma, and wherein the ABL1 fusion nucleic acid molecule: (a) comprises, in 5′ to 3′ direction, an ABL1 gene, or a portion thereof, fused to a NUP214 gene, or a portion thereof; (b) comprises a fusion between exon 1, or a portion thereof, of an ABL1 gene fused to exon 31, or a portion thereof, of a NUP214 gene; (c) comprises or results from a 5′ breakpoint within chromosomal coordinates chr9: 134090583-134090623 and/or a 3′ breakpoint within chromosomal coordinates chr9: 133710830-133710870; and/or (d) comprises, in 5′ to 3′ direction, exon 1, or a portion thereof, of an ABL1 gene, fused to exon 31, or a portion thereof, and exons 32-36 of NUP214. In some embodiments, the ABL1 fusion polypeptide encoded by the ABL1 fusion nucleic acid molecule comprises a fusion between an ABL1 polypeptide, or a portion thereof, fused to a NUP214 polypeptide, or a portion thereof, wherein the cancer is soft tissue liposarcoma, and wherein the ABL1 fusion polypeptide: (a) comprises, in the N- to C-terminus direction, an ABL1 polypeptide, or a portion thereof, fused to a NUP214 polypeptide, or a portion thereof; (b) comprises a fusion between an amino acid sequence encoded by exon 1, or a portion thereof, of an ABL1gene fused to an amino acid sequence encoded by exon 31, or a portion thereof, of a NUP214 gene; (c) results from a 5′ breakpoint within chromosomal coordinates chr9: 134090583-134090623 and/or a 3′ breakpoint within chromosomal coordinates chr9: 133710830-133710870; and/or (d) comprises, in the N- to C-terminus direction, an amino acid sequence encoded by exon 1, or a portion thereof, of an ABL1 gene, fused to an amino acid sequence encoded by exon 31, or a portion thereof, and exons 32-36 of a NUP214 gene.

In some embodiments, which may be combined with any of the preceding aspects or embodiments, the ABL1 fusion nucleic acid molecule comprises a fusion between an ABL1 gene, or a portion thereof, fused to a AIF1L gene, or a portion thereof, wherein the cancer is soft tissue sarcoma, and wherein the ABL1 fusion nucleic acid molecule: (a) comprises, in 5′ to 3′ direction, an ABL1 gene, or a portion thereof, fused to an AIF1L gene, or a portion thereof; (b) comprises a fusion between exon 10, or a portion thereof, of an ABL1 gene fused to exon 4, or a portion thereof, of an AIF1L gene; (c) comprises or results from a 5′ breakpoint within chromosomal coordinates chr9: 133755952-133755992 and/or a 3′ breakpoint within chromosomal coordinates chr9: 133989965-133990005; (d) comprises, in 5′ to 3′ direction, exons 1-9 and exon 10, or a portion thereof, of an ABL1 gene, fused to exon 4, or a portion thereof, and exons 5-6 of an AIF1L gene; (e) comprises the nucleotide sequence of SEQ ID NO: 20, or a nucleotide sequence having at least about 70% homology thereto; and/or (f) encodes an ABL1 fusion polypeptide comprising the amino acid sequence of SEQ ID NO: 41, or an amino acid sequence having at least about 70% homology thereto; optionally wherein the soft tissue sarcoma is not otherwise specified (NOS). In some embodiments, the ABL1 fusion polypeptide encoded by the ABL1 fusion nucleic acid molecule comprises a fusion between an ABL1 polypeptide, or a portion thereof, fused to an AIF1L polypeptide, or a portion thereof, wherein the cancer is soft tissue sarcoma, and wherein the ABL1 fusion polypeptide: (a) comprises, in the N- to C-terminus direction, an ABL1 polypeptide, or a portion thereof, fused to an AIF1L polypeptide, or a portion thereof; (b) comprises a fusion between an amino acid sequence encoded by exon 10, or a portion thereof, of an ABL1 gene fused to an amino acid sequence encoded by exon 4, or a portion thereof, of an AIF1L gene; (c) results from a 5′ breakpoint within chromosomal coordinates chr9: 133755952-133755992 and/or a 3′ breakpoint within chromosomal coordinates chr9: 133989965-133990005; (d) comprises, in the N- to C-terminus direction, an amino acid sequence encoded by exons 1-9 and exon 10, or a portion thereof, of an ABL1 gene fused to an amino acid sequence encoded by exon 4, or a portion thereof, and exons 5-6 of an AIF1L gene; (e) is encoded by the nucleotide sequence of SEQ ID NO: 20, or a nucleotide sequence having at least about 70% homology thereto; and/or (f) comprises the amino acid sequence of SEQ ID NO: 41, or an amino acid sequence having at least about 70% homology thereto; optionally wherein the soft tissue sarcoma is not otherwise specified (NOS).

In some embodiments, which may be combined with any of the preceding aspects or embodiments, the ABL1 fusion nucleic acid molecule comprises a fusion between an ABL1 gene, or a portion thereof, fused to a AIF1L gene, or a portion thereof, wherein the cancer is ovary serous carcinoma, and wherein the ABL1 fusion nucleic acid molecule: (a) comprises, in 5′ to 3′ direction, an AIF1L gene, or a portion thereof, fused to an ABL1 gene, or a portion thereof; (b) comprises a fusion between exon 2, or a portion thereof, of an AIF1L gene fused to exon 2, or a portion thereof, of an ABL1 gene; (c) comprises or results from a breakpoint within intron 2 of an AIF1L gene and/or a breakpoint within intron 1 of an ABL1 gene; (d) comprises or results from a 5′ breakpoint within chromosomal coordinates chr9: 133978032-133978137 and/or a 3′ breakpoint within chromosomal coordinates chr9: 133729409-133729618; and/or (e) comprises, in 5′ to 3′ direction, exon 1 and exon 2, or a portion thereof, of an AIF1L gene, fused to exon 2, or a portion thereof, and exons 3-11 of an ABL1 gene. In some embodiments, the ABL1 fusion polypeptide encoded by the ABL1 fusion nucleic acid molecule comprises a fusion between an ABL1 polypeptide, or a portion thereof, fused to a AIF1L polypeptide, or a portion thereof, wherein the cancer is ovary serous carcinoma, and wherein the ABL1 fusion polypeptide: (a) comprises, in the N- to C-terminus direction, an AIF1L polypeptide, or a portion thereof, fused to an ABL1 polypeptide, or a portion thereof; (b) comprises a fusion between an amino acid sequence encoded by exon 2, or a portion thereof, of an AIF1L gene fused to an amino acid sequence encoded by exon 2, or a portion thereof, of an ABL1 gene; (c) results from a breakpoint within intron 2 of an AIF1L gene and/or a breakpoint within intron 1 of an ABL1 gene; (d) results from a 5′ breakpoint within chromosomal coordinates chr9: 133978032-133978137 and/or a 3′ breakpoint within chromosomal coordinates chr9: 133729409-133729618; and/or (e) comprises, in the N- to C-terminus direction, an amino acid sequence encoded by exon 1 and exon 2, or a portion thereof, of an AIF1L gene fused to an amino acid sequence encoded by exon 2, or a portion thereof, and exons 3-11 of an ABL1 gene.

In some embodiments, which may be combined with any of the preceding aspects or embodiments, the ABL1 fusion nucleic acid molecule comprises a fusion between an ABL1 gene, or a portion thereof, fused to a RCSD1 gene, or a portion thereof, wherein the cancer is bone marrow leukemia lymphocytic chronic (CLL), and wherein the ABL1 fusion nucleic acid molecule: (a) comprises, in 5′ to 3′ direction an RCSD1 gene, or a portion thereof, fused to an ABL1 gene, or a portion thereof; (b) comprises a fusion between exon 4, or a portion thereof, of an RCSD1 gene fused to exon 4, or a portion thereof, of an ABL1 gene; (c) comprises or results from a 5′ breakpoint within chromosomal coordinates chr1: 167654668-167654708 and/or a 3′ breakpoint within chromosomal coordinates chr9: 133738173-133738213; (d) comprises, in 5′ to 3′ direction, exons 1-3 and exon 4, or a portion thereof, of a RCSD1 gene, fused to exon 4, or a portion thereof, and exons 5-11 of an ABL1 gene; (e) comprises the nucleotide sequence of SEQ ID NO: 21, or a nucleotide sequence having at least about 70% homology thereto; and/or (f) encodes an ABL1 fusion polypeptide comprising the amino acid sequence of SEQ ID NO: 42, or an amino acid sequence having at least about 70% homology thereto. In some embodiments, the ABL1 fusion polypeptide encoded by the ABL1 fusion nucleic acid molecule comprises a fusion between an ABL1 polypeptide, or a portion thereof, fused to a RCSD1 polypeptide, or a portion thereof, wherein the cancer is bone marrow leukemia lymphocytic chronic (CLL), and wherein the ABL1 fusion polypeptide: (a) comprises, in the N- to C-terminus direction, an RCSD1 polypeptide, or a portion thereof, fused to an ABL1 polypeptide, or a portion thereof; (b) comprises a fusion between an amino acid sequence encoded by exon 4, or a portion thereof, of an RCSD1 gene fused to an amino acid sequence encoded by exon 4, or a portion thereof, of an ABL1 gene; (c) results from a 5′ breakpoint within chromosomal coordinates chr1: 167654668-167654708 and/or a 3′ breakpoint within chromosomal coordinates chr9: 133738173-133738213; (d) comprises, in the N- to C-terminus direction, an amino acid sequence encoded by exons 1-3 and exon 4, or a portion thereof, of an RCSD1 gene, fused to an amino acid sequence encoded by exon 4, or a portion thereof, and exons 5-11 of an ABL1 gene; (e) is encoded by the nucleotide sequence of SEQ ID NO: 21, or a nucleotide sequence having at least about 70% homology thereto; and/or (f) comprises the amino acid sequence of SEQ ID NO: 42, or an amino acid sequence having at least about 70% homology thereto.

In some embodiments, which may be combined with any of the preceding aspects or embodiments, the ABL1 fusion nucleic acid molecule comprises a fusion between an ABL1 gene, or a portion thereof, fused to an EXOSC2 gene, or a portion thereof, wherein the cancer is peritoneum adenocarcinoma, and wherein the ABL1 fusion nucleic acid molecule: (a) comprises, in 5′ to 3′ direction, an EXOSC2 gene, or a portion thereof, fused to an ABL1 gene, or a portion thereof; (b) comprises a fusion between exon 9, or a portion thereof, of an EXOSC2 gene fused to exon 1, or a portion thereof, of an ABL1 gene; (c) comprises or results from a breakpoint within exon 9 of an EXOSC2 gene and/or a breakpoint within exon 1 of an ABL1 gene; (d) comprises or results from a 5′ breakpoint within chromosomal coordinates chr9: 133579154-133579531 and/or a 3′ breakpoint within chromosomal coordinates chr9: 133589786-133589985; and/or (e) comprises, in 5′ to 3′ direction, exons 1-8 and exon 9, or a portion thereof, of an EXOSC2 gene, fused to exon 1, or a portion thereof, and exons 2-11 of an ABL1 gene. In some embodiments, the ABL1 fusion polypeptide encoded by the ABL1 fusion nucleic acid molecule comprises a fusion between an ABL1 polypeptide, or a portion thereof, fused to an EXOSC2 polypeptide, or a portion thereof, wherein the cancer is peritoneum adenocarcinoma, and wherein the ABL1 fusion polypeptide: (a) comprises, in the N- to C-terminus direction, an EXOSC2 polypeptide, or a portion thereof, fused to an ABL1 polypeptide, or a portion thereof; (b) comprises a fusion between an amino acid sequence encoded by exon 9, or a portion thereof, of an EXOSC2 gene fused to an amino acid sequence encoded by exon 1, or a portion thereof, of an ABL1 gene; (c) results from a breakpoint within exon 9 of an EXOSC2 gene and a breakpoint within exon 1 of an ABL1 gene; (d) results from a 5′ breakpoint within chromosomal coordinates chr9: 133579154-133579531 and/or a 3′ breakpoint within chromosomal coordinates chr9: 133589786-133589985; and/or (e) comprises, in the N- to C-terminus direction, an amino acid sequence encoded by exons 1-8 and exon 9, or a portion thereof, of an EXOSC2 gene fused to an amino acid sequence encoded by exon 1, or a portion thereof, and exons 2-11 of an ABL1 gene.

In another aspect, provided herein is a kit comprising one or more probes, baits, or oligonucleotides for detecting an ABL1 fusion nucleic acid molecule comprising a fusion between an ABL1 gene, or a portion thereof, fused to: (a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; (b) a second gene, or a portion of a second gene, wherein the ABL1 fusion nucleic acid molecule comprises or results from an ABL1 breakpoint within any of exons 1-4 and 10, or introns 4-5, of ABL1; (c) a BCR gene, or a portion thereof, wherein the kit is for detecting the ABL1 fusion nucleic acid molecule in a sample from an individual having a cancer, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC); (d) a NUP214 gene, or a portion thereof, wherein the kit is for detecting the ABL1 fusion nucleic acid molecule in a sample from an individual having a cancer, wherein the cancer is bone marrow leukemia lymphocytic chronic (CLL) or soft tissue liposarcoma; (e) an AIF1L gene, or a portion thereof, wherein the kit is for detecting the ABL1 fusion nucleic acid molecule in a sample from an individual having a cancer, wherein the cancer is soft tissue sarcoma, or ovary serous carcinoma; (f) an RCSD1 gene, or a portion thereof, wherein the kit is for detecting the ABL1 fusion nucleic acid molecule in a sample from an individual having a cancer, wherein the cancer is bone marrow leukemia lymphocytic chronic (CLL); or (g) an EXOSC2 gene, or a portion thereof, wherein the kit is for detecting the ABL1 fusion nucleic acid molecule in a sample from an individual having a cancer, wherein the cancer is peritoneum adenocarcinoma.

In another aspect, provided herein is a nucleic acid encoding an ABL1 fusion nucleic acid molecule, or a fragment thereof, comprising a fusion between an ABL1 gene, or a portion thereof, fused to: (a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; or (b) a second gene, or a portion of a second gene, wherein the ABL1 fusion nucleic acid molecule comprises or results from an ABL1 breakpoint within any of exons 1-4 and 10, or introns 4-5, of ABL1. In some embodiments, which may be combined with any of the preceding aspects or embodiments, a vector of the disclosure comprises a nucleic acid provided herein. In some embodiments, a host cell of the disclosure comprises a vector provided herein.

In another aspect, provided herein is an antibody or antibody fragment that specifically binds to an ABL1 fusion polypeptide, or to a portion thereof, wherein the ABL1 fusion polypeptide is encoded by an ABL1 fusion nucleic acid molecule comprising a fusion between an ABL1 gene, or a portion thereof, fused to: (a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; or (b) a second gene, or a portion of a second gene, wherein the ABL1 fusion nucleic acid molecule comprises or results from an ABL1 breakpoint within any of exons 1-4 and 10, or introns 4-5, of ABL1.

In another aspect, provided herein is a kit comprising an antibody or antibody fragment of the disclosure.

In another aspect, provided herein is an in vitro use of one or more probes, baits, or oligonucleotides for detecting an ABL1 fusion nucleic acid molecule comprising a fusion between an ABL1 gene, or a portion thereof, fused to: (a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; (b) a second gene, or a portion of a second gene, wherein the ABL1 fusion nucleic acid molecule comprises or results from an ABL1 breakpoint within any of exons 1-4 and 10, or introns 4-5, of ABL1; (c) a BCR gene, or a portion thereof, wherein the one or more probes, baits, or oligonucleotides are for use in detecting the ABL1 fusion nucleic acid molecule in a sample from an individual having a cancer, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC); (d) a NUP214 gene, or a portion thereof, wherein the one or more probes, baits, or oligonucleotides are for use in detecting the ABL1 fusion nucleic acid molecule in a sample from an individual having a cancer, wherein the cancer is bone marrow leukemia lymphocytic chronic (CLL) or soft tissue liposarcoma; (e) an AIF1L gene, or a portion thereof, wherein the one or more probes, baits, or oligonucleotides are for use in detecting the ABL1 fusion nucleic acid molecule in a sample from an individual having a cancer, wherein the cancer is soft tissue sarcoma, or ovary serous carcinoma; (f) an RCSD1 gene, or a portion thereof, wherein the one or more probes, baits, or oligonucleotides are for use in detecting the ABL1 fusion nucleic acid molecule in a sample from an individual having a cancer, wherein the cancer is bone marrow leukemia lymphocytic chronic (CLL); or (g) an EXOSC2 gene, or a portion thereof, wherein the one or more probes, baits, or oligonucleotides are for use in detecting the ABL1 fusion nucleic acid molecule in a sample from an individual having a cancer, wherein the cancer is peritoneum adenocarcinoma.

In another aspect, provided herein is an ABL1-targeted therapy for use in a method of treating or delaying progression of cancer, wherein the method comprises administering the ABL1-targeted therapy to an individual having a cancer, wherein: (1) an ABL1 fusion nucleic acid molecule comprising a fusion between an ABL1 gene, or a portion thereof, fused to: (a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; (b) a second gene, or a portion of a second gene, wherein the ABL1 fusion nucleic acid molecule comprises or results from an ABL1 breakpoint within any of exons 1-4 and 10, or introns 4-5, of ABL1; (c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC); (d) a NUP214 gene, or a portion thereof, wherein the cancer is bone marrow leukemia lymphocytic chronic (CLL) or soft tissue liposarcoma; (e) an AIF1L gene, or a portion thereof, wherein the cancer is soft tissue sarcoma, or ovary serous carcinoma; (f) an RCSD1 gene, or a portion thereof, wherein the cancer is bone marrow leukemia lymphocytic chronic (CLL); or (g) an EXOSC2 gene, or a portion thereof, wherein the cancer is peritoneum adenocarcinoma; or (2) an ABL1 fusion polypeptide encoded by the ABL1 fusion nucleic acid molecule, is detected in a sample obtained from the individual.

In another aspect, provided herein is an ABL1-targeted therapy for use in the manufacture of a medicament for treating or delaying progression of cancer, wherein the medicament is to be administered to an individual having a cancer, wherein: (1) an ABL1 fusion nucleic acid molecule comprising a fusion between an ABL1 gene, or a portion thereof, fused to: (a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; (b) a second gene, or a portion of a second gene, wherein the ABL1 fusion nucleic acid molecule comprises or results from an ABL1 breakpoint within any of exons 1-4 and 10, or introns 4-5, of ABL1; (c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC); (d) a NUP214 gene, or a portion thereof, wherein the cancer is bone marrow leukemia lymphocytic chronic (CLL) or soft tissue liposarcoma; (e) an AIF1L gene, or a portion thereof, wherein the cancer is soft tissue sarcoma, or ovary serous carcinoma;

(f) an RCSD1 gene, or a portion thereof, wherein the cancer is bone marrow leukemia lymphocytic chronic (CLL); or (g) an EXOSC2 gene, or a portion thereof, wherein the cancer is peritoneum adenocarcinoma; or (2) an ABL1 fusion polypeptide encoded by the ABL1 fusion nucleic acid molecule, is detected in a sample obtained from the individual.

In another aspect, provided herein is a system comprising: a memory configured to store one or more program instructions, and one or more processors configured to execute the one or more program instructions, the one or more program instructions when executed by the one or more processors are configured to: (a) obtain a plurality of sequence reads of one or more nucleic acid molecules, wherein the one or more nucleic acid molecules are derived from a sample obtained from an individual having a cancer; (b) analyze the plurality of sequence reads for the presence of an ABL1 fusion nucleic acid molecule comprising a fusion between an ABL1 gene, or a portion thereof, fused to: (i) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; (ii) a second gene, or a portion of a second gene, wherein the ABL1 fusion nucleic acid molecule comprises or results from an ABL1 breakpoint within any of exons 1-4 and 10, or introns 4-5, of ABL1; (iii) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC); (iv) a NUP214 gene, or a portion thereof, wherein the cancer is bone marrow leukemia lymphocytic chronic (CLL) or soft tissue liposarcoma; (v) an AIF1L gene, or a portion thereof, wherein the cancer is soft tissue sarcoma, or ovary serous carcinoma; (vi) an RCSD1 gene, or a portion thereof, wherein the cancer is bone marrow leukemia lymphocytic chronic (CLL); or (vii) an EXOSC2 gene, or a portion thereof, wherein the cancer is peritoneum adenocarcinoma; and (c) detect, based on the analyzing, the ABL1 fusion nucleic acid molecule in the sample.

In another aspect, provided herein is a non-transitory computer readable storage medium comprising one or more programs executable by one or more computer processors for performing a method, the method comprising: (a) obtaining, using the one or more processors, a plurality of sequence reads of one or more nucleic acid molecules, wherein the one or more nucleic acid molecules are derived from a sample obtained from an individual having a cancer; (b) analyzing, using the one or more processors, the plurality of sequence reads for the presence of an ABL1 fusion nucleic acid molecule comprising a fusion between an ABL1 gene, or a portion thereof, fused to: (i) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; (ii) a second gene, or a portion of a second gene, wherein the ABL1 fusion nucleic acid molecule comprises or results from an ABL1 breakpoint within any of exons 1-4 and 10, or introns 4-5, of ABL1; (iii) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC); (iv) a NUP214 gene, or a portion thereof, wherein the cancer is bone marrow leukemia lymphocytic chronic (CLL) or soft tissue liposarcoma; (v) an AIF1L gene, or a portion thereof, wherein the cancer is soft tissue sarcoma, or ovary serous carcinoma; (vi) an RCSD1 gene, or a portion thereof, wherein the cancer is bone marrow leukemia lymphocytic chronic (CLL); or (vii) an EXOSC2 gene, or a portion thereof, wherein the cancer is peritoneum adenocarcinoma; and (c) detecting, using the one or more processors and based on the analyzing, the ABL1 fusion nucleic acid molecule in the sample.

In some embodiments, which may be combined with any of the preceding aspects or embodiments, the plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next generation sequencing (NGS).

In some embodiments, which may be combined with any of the preceding aspects or embodiments, the one or more program instructions when executed by the one or more processors are further configured to generate, based at least in part on the detecting, a molecular profile for the sample. In some embodiments, the method further comprises generating, based at least in part on the detecting, a molecular profile for the sample. In some embodiments, the individual is administered a treatment based at least in part on the molecular profile; optionally wherein the treatment comprises an ABL1-targeted therapy. In some embodiments, the molecular profile further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the molecular profile further comprises results from a nucleic acid sequencing-based test.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 depicts an exemplary device, in accordance with some embodiments.

FIG. 2 depicts an exemplary system, in accordance with some embodiments.

FIG. 3 depicts a block diagram of an exemplary process for detecting an ABL1 fusion nucleic acid molecule, in accordance with some embodiments.

### DETAILED DESCRIPTION

The present disclosure relates generally to detecting Abelson tyrosine-protein kinase 1 (ABL1) fusions in cancer, as well as methods of treatment, and uses related thereto.

Kinase fusions are an important class of targetable oncogenic driver variants. The present disclosure describes the results of comprehensive genomic profiling (CGP) of the pan-cancer landscape of ABL1 gene fusions. These analyses identified diverse rearrangements leading to fusion genes involving ABL1 and numerous fusion partner genes (see, e.g., Example 1). Without wishing to be bound by theory, it is thought that the presence of an ABL1 fusion described herein in a sample from individuals having cancer may identify cancer patients who are likely to respond to treatment with an anti-cancer therapy such as a targeted anti-cancer therapy, e.g., an ABL1-targeted therapy as described herein.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. De Vita et al., eds., J. B. Lippincott Company, 1993).

### II. Definitions

As used in this specification and the appended claims, the singular forms “a”, “an” and “the” include plural referents unless the context clearly dictates otherwise. Thus, for example, reference to “a molecule” optionally includes a combination of two or more such molecules, and the like.

The terms “about” and “approximately” as used herein refer to the usual error range for the respective value readily known to the skilled person in this technical field. Exemplary degrees of error are within 20 percent (%), typically, within 10%, and more typically, within 5% of a given value or range of values. Reference to “about” or “approximately” a value or parameter herein includes (and describes) embodiments that are directed to that value or parameter per se.

It is understood that aspects and embodiments of the invention described herein include “comprising,” “consisting,” and “consisting essentially of” aspects and embodiments.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

“Polynucleotide,” “nucleic acid,” or “nucleic acid molecule” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(0)S (“thioate”), P(S)S (“dithioate”), “(0)NR₂ (“amidate”), P(0)R, P(0)OR′, CO or CH₂ (“formacetal”), in which each R or R′ is independently H or substituted or unsubstituted alkyl (1-20 C) optionally containing an ether (-0-) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “isolated” antibody is one which has been identified and separated and/or recovered from a component of its natural environment. Contaminant components of its natural environment are materials which would interfere with research, diagnostic, and/or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. An isolated antibody includes the antibody in situ within recombinant cells since at least one component of the antibody's natural environment will not be present. Ordinarily, however, an isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The “light chains” of antibodies (immunoglobulins) from any mammalian species can be assigned to one of two clearly distinct types, called kappa (“κ”) and lambda (“λ”), based on the amino acid sequences of their constant domains.

The term “constant domain” refers to the portion of an immunoglobulin molecule having a more conserved amino acid sequence relative to the other portion of the immunoglobulin, the variable domain, which contains the antigen binding site. The constant domain contains the CH1, CH2, and CH3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The term “hypervariable region,” “HVR,” or “HV,” as used herein, refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, for example, Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, for example, Hamers-Casterman et al., Nature 363:446-448 (1 993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1 991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat numbering) |
| H1 | H31-H35 | H26-H35 | H26-H32 | H30-H35 (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-1 13 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human lgG1 EU antibody.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody comprising the antigen-binding region thereof. In some embodiments, the antibody fragment described herein is an antigen-binding fragment. Examples of antibody fragments include Fab, Fab′, F(ab′)2, and Fv fragments; diabodies; linear antibodies; single-chain antibody molecules; and multispecific antibodies formed from antibody fragments.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target-binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target-binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target-binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature 256:495-97 (1975); Hongo et al., Hybridoma 14 (3): 253-260 (1995), Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352:624-628 (1991); Marks et al., J. Mol. Biol. 222:581-597 (1992); Sidhu et al., J. Mol. Biol. 338 (2): 299-31 0 (2004); Lee et al., J. Mol. Biol. 340 (5): 1073-1093 (2004); Fellouse, Proc. Natl. Acad. Sci. USA 101 (34): 12467-12472 (2004); and Lee et al., J. Immunol. Methods 284 (1-2): 1 1 9-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90:2551 (1993); Jakobovits et al., Nature 362:255-258 (1993); Bruggemann et al., Year in Immunol. 7:33 (1 993); U.S. Pat. Nos. 5,545,807; 5,545,806; 5,569,825; 5,625,126; 5,633,425; and U.S. Pat. No. 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg et al., Intern. Rev. Immunol. 13:65-93 (1995)).

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human framework regions (FRs). In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody.

A “humanized form” of an antibody, e.g., a non-human antibody, refers to an antibody that has undergone humanization.

A “blocking” antibody or an “antagonist” antibody is one which inhibits or reduces biological activity of the antigen it binds. For example, blocking antibodies or antagonist antibodies substantially or completely inhibit the biological activity of the antigen.

As used herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of <1 μM, <100 nM, <10 nM, <1 nM, or <0.1 nM. In certain embodiments, an antibody specifically binds to an epitope on a protein that is conserved among the protein from different species. In another embodiment, specific binding can include, but does not require exclusive binding.

The terms “homology” or “identity,” as used herein, refer to sequence similarity between two polynucleotide sequences or between two polypeptide sequences. The phrases “percent identity or homology” and “% identity or homology” refer to the percentage of sequence similarity found in a comparison of two or more polynucleotide sequences or two or more polypeptide sequences. Identity or similarity can be determined by comparing a position in each sequence that can be aligned for purposes of comparison. When a position in the compared sequences is occupied by the same nucleotide base or amino acid, then the molecules are identical at that position.

The term “detection” includes any means of detecting, including direct and indirect detection. The term “biomarker” as used herein (e.g., a “biomarker” such as an ABL1 fusion or an ABL1 fusion nucleic acid molecule or polypeptide described herein) refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features (e.g., responsiveness to therapy). In some embodiments, a biomarker is a collection of genes or a collective number of mutations/alterations (e.g., somatic mutations) in a collection of genes. Biomarkers include, but are not limited to, polynucleotides (e.g., DNA and/or RNA), polynucleotide alterations (e.g., polynucleotide copy number alterations, e.g., DNA copy number alterations), polypeptides, polypeptide and polynucleotide modifications (e.g., post-translational modifications), carbohydrates, and/or glycolipid-based molecular markers.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)).

The term “aiding diagnosis” is used herein to refer to methods that assist in making a clinical determination regarding the presence, or nature, of a particular type of symptom or condition of a disease or disorder (e.g., cancer). For example, a method of aiding diagnosis of a disease or condition (e.g., cancer) can comprise measuring certain somatic mutations in a biological sample from an individual.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In some instances, the sample is a whole blood sample, a plasma sample, a serum sample, or a combination thereof. In some embodiments, the sample is from a tumor (e.g., a “tumor sample”), such as from a biopsy. In some embodiments, the sample is a formalin-fixed paraffin-embedded (FFPE) sample.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” or “control tissue,” as used herein, refer to a sample, cell, tissue, standard, or level that is used for comparison purposes.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

An “effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and in some embodiments stop) cancer cell infiltration into peripheral organs; inhibit (i.e., slow to some extent and in some embodiments stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), response rates (e.g., CR and PR), duration of response, and/or quality of life.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the patient herein is a human.

As used herein, “administering” is meant a method of giving a dosage of an agent or a pharmaceutical composition (e.g., a pharmaceutical composition including the agent) to a subject (e.g., a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

The term “package insert” is used to refer to instructions customarily included in commercial packages of therapeutic products, that contain information about the indications, usage, dosage, administration, combination therapy, contraindications, and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a reagent for specifically detecting a biomarker (e.g., an ABL1 fusion nucleic acid molecule or polypeptide described herein) described herein. In certain embodiments, the manufacture or kit is promoted, distributed, or sold as a unit for performing the methods described herein.

The phrase “based on”, “responsive to”, and the like, when used herein mean that the information about one or more biomarkers (e.g., an ABL1 fusion or an ABL1 fusion nucleic acid molecule or polypeptide described herein) is used to inform a treatment decision, information provided on a package insert, or marketing/promotional guidance, etc.

The terms “allele frequency” and “allele fraction” are used interchangeably herein and refer to the fraction of sequence reads corresponding to a particular allele relative to the total number of sequence reads for a genomic locus. The terms “variant allele frequency” and “variant allele fraction” are used interchangeably herein and refer to the fraction of sequence reads corresponding to a particular variant allele relative to the total number of sequence reads for a genomic locus.

### III. Methods, Systems, and Devices

In certain aspects, provided herein are methods for selecting a treatment for an individual having a cancer; methods for identifying one or more treatment options for an individual having a cancer; methods for predicting survival of an individual having a cancer; methods for treating or delaying progression of cancer; methods for monitoring, evaluating or screening an individual having a cancer; methods for assessing an Abelson tyrosine-protein kinase 1 (ABL1) fusion nucleic acid molecule or an ABL1 fusion polypeptide encoded by an ABL1 fusion nucleic acid molecule in a cancer in an individual; methods for detecting an ABL1 fusion nucleic acid molecule or an ABL1 fusion polypeptide encoded by an ABL1 fusion nucleic acid molecule; methods for detecting the presence or absence of a cancer in an individual; methods for monitoring progression or recurrence of a cancer in an individual; methods for identifying a candidate treatment for a cancer in an individual in need thereof; methods for identifying an individual having a cancer who may benefit from a treatment comprising an anti-cancer therapy, such as an ABL1-targeted therapy; and methods for predicting survival of an individual having a cancer treated with a treatment comprising an anti-cancer therapy, such as an ABL1-targeted therapy.

In some embodiments, the methods provided herein comprise detecting in a sample from an individual, e.g., an individual having cancer, suspected of having cancer, being treated for cancer, or being tested for cancer, an ABL1 fusion nucleic acid molecule, or an ABL1 fusion polypeptide encoded by an ABL1 fusion nucleic acid molecule. In some embodiments, detection of the ABL1 fusion nucleic acid molecule, or the ABL1 fusion polypeptide, in the sample identifies the individual as one who may benefit from a treatment comprising an anti-cancer therapy, such as an ABL1-targeted therapy. In some embodiments, the methods comprise selecting an anti-cancer therapy as a treatment for an individual having cancer, e.g., responsive to detection of an ABL1 fusion nucleic acid molecule, or an ABL1 fusion polypeptide encoded by an ABL1 fusion nucleic acid molecule, in a sample from an individual, e.g., an individual having cancer, suspected of having cancer, being treated for cancer, or being tested for cancer. In some embodiments, the methods comprise generating a report comprising one or more treatment options identified for an individual (e.g., an individual having cancer, suspected of having cancer, being treated for cancer, or being tested for cancer) based at least in part on detection of an ABL1 fusion nucleic acid molecule, or an ABL1 fusion polypeptide encoded by an ABL1 fusion nucleic acid molecule, in a sample from the individual. In some embodiments, the one or more treatment options comprise an anti-cancer therapy as described herein, such as an ABL1-targeted therapy. In some embodiments, the methods comprise administering to an individual (e.g., an individual having cancer, suspected of having cancer, being treated for cancer, or being tested for cancer) an effective amount of a treatment that comprises an anti-cancer therapy, such as an ABL1-targeted therapy, responsive to detecting an ABL1 fusion nucleic acid molecule, or an ABL1 fusion polypeptide encoded by an ABL1 fusion nucleic acid molecule, in a sample from the individual. In some embodiments, responsive to detection of an ABL1 fusion nucleic acid molecule, or an ABL1 fusion polypeptide encoded by an ABL1 fusion nucleic acid molecule, in a sample from an individual (e.g., an individual having cancer, suspected of having cancer, being treated for cancer, or being tested for cancer), the individual is predicted to have longer survival when treated with a treatment comprising an anti-cancer therapy, such as an ABL1-targeted therapy, as compared to survival of an individual whose cancer does not comprise an ABL1 fusion nucleic acid molecule or an ABL1 fusion polypeptide. In some embodiments, the methods comprise providing an assessment of an ABL1 fusion nucleic acid molecule, or an ABL1 fusion polypeptide encoded by an ABL1 fusion nucleic acid molecule, e.g., responsive to detecting the presence or absence of the ABL1 fusion nucleic acid molecule, or the ABL1 fusion polypeptide, in a sample. In some embodiments, the methods comprise detecting or acquiring knowledge of the presence or absence of a cancer in a sample from the individual. In some embodiments, the methods comprise detecting, in a first sample obtained from an individual (e.g., an individual having cancer, suspected of having cancer, being treated for cancer, or being tested for cancer) at a first time point, the presence or absence of an ABL1 fusion nucleic acid molecule, or an ABL1 fusion polypeptide encoded by an ABL1 fusion nucleic acid molecule; detecting, in a second sample obtained from the individual at a second time point after the first time point, the presence or absence of an ABL1 fusion nucleic acid molecule, or an ABL1 fusion polypeptide encoded by an ABL1 fusion nucleic acid molecule; and providing an assessment of cancer progression or cancer recurrence in the individual based, at least in part, on the presence or absence of the ABL1 fusion nucleic acid molecule, or the ABL1 fusion polypeptide encoded by the ABL1 fusion nucleic acid molecule, in the first sample and/or in the second sample. In some embodiments, the methods comprise performing DNA sequencing on a sample obtained from an individual to determine a sequencing mutation profile, wherein the sequencing mutation profile identifies the presence or absence of an ABL1 fusion nucleic acid molecule. In some embodiments, the methods comprise identifying a candidate treatment based, at least in part, on the sequencing mutation profile. In some embodiments, the candidate treatment comprises an anti-cancer therapy described herein, such as an ABL1-targeted therapy. In some embodiments, the candidate treatment is identified based, at least in part, on the presence of an ABL1 fusion nucleic acid molecule as identified in the sequencing mutation profile.

In some embodiments, the methods provided herein comprise acquiring knowledge of an ABL1 fusion nucleic acid molecule, or an ABL1 fusion polypeptide encoded by an ABL1 fusion nucleic acid molecule, in a sample from an individual, e.g., an individual having cancer, suspected of having cancer, being treated for cancer, or being tested for cancer. In some embodiments, knowledge of the presence of an ABL1 fusion nucleic acid molecule, or an ABL1 fusion polypeptide encoded by the ABL1 fusion nucleic acid molecule, in a sample from an individual (e.g., an individual having cancer, suspected of having cancer, being treated for cancer, or being tested for cancer) identifies the individual as one who may benefit from a treatment comprising an anti-cancer therapy, such as an ABL1-targeted therapy. In some embodiments, the methods comprise selecting an anti-cancer therapy, such as an ABL1-targeted therapy, as a treatment for an individual (e.g., an individual having cancer, suspected of having cancer, being treated for cancer, or being tested for cancer), e.g., responsive to knowledge of the presence of an ABL1 fusion nucleic acid molecule, or an ABL1 fusion polypeptide encoded by an ABL1 fusion nucleic acid molecule, in a sample from the individual. In some embodiments, the methods comprise generating a report comprising one or more treatment options identified for an individual (e.g., an individual having cancer, suspected of having cancer, being treated for cancer, or being tested for cancer) based at least in part on knowledge of the presence of an ABL1 fusion nucleic acid molecule, or an ABL1 fusion polypeptide encoded by an ABL1 fusion nucleic acid molecule in sample from the individual. In some embodiments, the one or more treatment options comprise an anti-cancer therapy described herein, such as an ABL1-targeted therapy. In some embodiments, responsive to acquisition of knowledge of the presence of an ABL1 fusion nucleic acid molecule, or an ABL1 fusion polypeptide encoded by an ABL1 fusion nucleic acid molecule, in a sample from an individual (e.g., an individual having cancer, suspected of having cancer, being treated for cancer, or being tested for cancer), the individual is classified as a candidate to receive a treatment comprising an anti-cancer therapy, e.g., such as an ABL1-targeted therapy. In some embodiments, responsive to acquisition of knowledge of the presence of an ABL1 fusion nucleic acid molecule, or an ABL1 fusion polypeptide encoded by an ABL1 fusion nucleic acid molecule, in a sample from an individual (e.g., an individual having cancer, suspected of having cancer, being treated for cancer, or being tested for cancer), the individual is identified as likely to respond to a treatment that comprises an anti-cancer therapy, such as an ABL1-targeted therapy. In some embodiments, responsive to acquisition of knowledge of the presence of an ABL1 fusion nucleic acid molecule, or an ABL1 fusion polypeptide encoded by an ABL1 fusion nucleic acid molecule, in a sample from an individual, the individual is predicted to have longer survival when treated with a treatment comprising an anti-cancer therapy, such as an ABL1-targeted therapy, e.g., as compared to survival of an individual whose cancer does not comprise an ABL1 fusion nucleic acid molecule, or an ABL1 fusion polypeptide. In some embodiments, responsive to acquisition of knowledge of the presence of an ABL1 fusion nucleic acid molecule, or an ABL1 fusion polypeptide encoded by an ABL1 fusion nucleic acid molecule, in a sample from an individual (e.g., an individual having cancer, suspected of having cancer, being treated for cancer, or being tested for cancer), the individual is predicted to have longer survival when treated with a treatment comprising an anti-cancer therapy, such as an ABL1-targeted therapy, as compared to an individual whose cancer does not exhibit an ABL1 fusion nucleic acid molecule, or an ABL1 fusion polypeptide. In some embodiments, the methods comprise administering to an individual (e.g., an individual having cancer, suspected of having cancer, being treated for cancer, or being tested for cancer) an effective amount of a treatment that comprises an anti-cancer therapy, such as an ABL1-targeted therapy, responsive to acquiring knowledge of an ABL1 fusion nucleic acid molecule, or an ABL1 fusion polypeptide encoded by an ABL1 fusion nucleic acid molecule, in a sample from the individual. In some embodiments, responsive to acquiring knowledge of an ABL1 fusion nucleic acid molecule, or an ABL1 fusion polypeptide encoded by an ABL1 fusion nucleic acid molecule, in a sample from an individual, the individual is predicted to have an improved response to treatment with an anti-cancer therapy, such as an ABL1-targeted therapy, as compared to an individual whose cancer does not comprise an ABL1 fusion nucleic acid molecule, or an ABL1 fusion polypeptide encoded by an ABL1 fusion nucleic acid molecule. In some embodiments, the methods comprise providing an assessment of an ABL1 fusion nucleic acid molecule, or an ABL1 fusion polypeptide encoded by an ABL1 fusion nucleic acid molecule, e.g., responsive to acquiring knowledge of the presence or absence of the ABL1 fusion nucleic acid molecule, or the ABL1 fusion polypeptide in a sample from an individual (e.g., an individual having cancer, suspected of having cancer, being treated for cancer, or being tested for cancer). In some embodiments, the methods comprise detecting or acquiring knowledge of the presence or absence of a cancer in a sample from an individual.

In other aspects, provided herein are systems. In some embodiments, a system of the disclosure comprises a memory configured to store one or more program instructions; and one or more processors configured to execute the one or more program instructions. In some embodiments, the one or more program instructions when executed by the one or more processors are configured to: (a) obtain a plurality of sequence reads of one or more nucleic acid molecules, wherein the one or more nucleic acid molecules are derived from a sample obtained from an individual (e.g., an individual having cancer, suspected of having cancer, being treated for cancer, or being tested for cancer); (b) analyze the plurality of sequence reads for the presence of an ABL1 fusion nucleic acid molecule; and (c) detect, based on the analyzing, the ABL1 fusion nucleic acid molecule in the sample.

In other aspects, provided herein are non-transitory computer readable storage media. In some embodiments, a non-transitory computer readable storage medium of the disclosure comprises one or more programs executable by one or more computer processors for performing a method. In some embodiments, the method comprises (a) obtaining, using the one or more processors, a plurality of sequence reads of one or more nucleic acid molecules, wherein the one or more nucleic acid molecules are derived from a sample obtained from an individual (e.g., an individual having cancer, suspected of having cancer, being treated for cancer, or being tested for cancer); (b) analyzing, using the one or more processors, the plurality of sequence reads for the presence of an ABL1 fusion nucleic acid molecule; and (c) detecting, using the one or more processors and based on the analyzing, the ABL1 fusion nucleic acid molecule in the sample.

In some embodiments of any of the methods, systems, or non-transitory computer readable storage media provided herein, the ABL1 fusion nucleic acid molecule comprises a fusion between an ABL1 gene, or a portion thereof, fused to any one of genes BMP2K, EHMT1, MED27, RFFL, SLC27A4, BCR, NUP214, AIF1L, RCSD1, APOOL, DNAH2, RHEX/C1orf186, RALGPS1, TSC1, TSPAN18, ZNF804B, EXOSC2, or SNX2, or a portion thereof, as described in detail below. In other embodiments of any of the methods, systems, or non-transitory computer readable storage media provided herein, the ABL1 fusion nucleic acid molecule comprises a fusion between an ABL1 gene, or a portion thereof, fused to a second gene, or a portion of a second gene, wherein the ABL1 fusion nucleic acid molecule comprises or results from an ABL1 breakpoint within any of exons 1-4 and 10, or introns 4-5, of ABL1, as described in detail below.

### A. ABL1 Fusions

Certain aspects of the present disclosure relate to genomic rearrangements involving an Abelson tyrosine-protein kinase 1 (ABL1) gene. An ABL1 rearrangement of the present disclosure may relate to any chromosomal translocation, fusion, or rearrangement involving the locus of an ABL1 gene. In some embodiments, the rearrangements of the disclosure result in an ABL1 fusion nucleic acid molecule that comprises at least a portion of an ABL1 gene fused to at least a portion of another gene. Accordingly, certain aspects of the present disclosure relate to ABL1 fusion nucleic acid molecules, as well as to ABL1 fusion polypeptides encoded by such ABL1 fusion nucleic acid molecules.

As used herein “Abelson tyrosine-protein kinase 1” or “ABL1” refer to a gene encoding an ABL1 mRNA or polypeptide. The ABL1 gene encodes a tyrosine-protein kinase. ABL1 is also known as ABL, BCR-ABL, bcr/abl, c-ABL, c-ABL1, CHDSKM, JTK7, p150, and v-abl. In some embodiments, an ABL1 gene according to the present disclosure is a human ABL1 gene. An exemplary ABL1 gene is represented by NCBI Gene ID No. 25. Exemplary ABL1 mRNA sequences are represented by NCBI Ref. Seqs. NM_005157 and NM_007313, provided below as SEQ ID NOS: 1 and 79, respectively.

(SEQ ID NO: 1)
GTTAACAGGCGCGTCCCGGCCAGGCGGAGACGCGGCCGCGGCCATGGGCGGGCGCGGGCGCGCGGGGC
GGCGGTGAGGGCGGCTGGCGGGGCCGGGGGCGCCGGGGGGGCGCGCGGGCCGAGCCGGGCCTGAGCCG
GGCCCGCGGACCGAGCTGGGAGAGGGGTTCCGGCCCCCGACGTGCTGGCGCGGGAAAATGTTGGAGAT
CTGCCTGAAGCTGGTGGGCTGCAAATCCAAGAAGGGGCTGTCCTCGTCCTCCAGCTGTTATCTGGAAG
AAGCCCTTCAGCGGCCAGTAGCATCTGACTTTGAGCCTCAGGGTCTGAGTGAAGCCGCTCGTTGGAAC
TCCAAGGAAAACCTTCTCGCTGGACCCAGTGAAAATGACCCCAACCTTTTCGTTGCACTGTATGATTT
TGTGGCCAGTGGAGATAACACTCTAAGCATAACTAAAGGTGAAAAGCTCCGGGTCTTAGGCTATAATC
ACAATGGGGAATGGTGTGAAGCCCAAACCAAAAATGGCCAAGGCTGGGTCCCAAGCAACTACATCACG
CCAGTCAACAGTCTGGAGAAACACTCCTGGTACCATGGGCCTGTGTCCCGCAATGCCGCTGAGTATCT
GCTGAGCAGCGGGATCAATGGCAGCTTCTTGGTGCGTGAGAGTGAGAGCAGTCCTGGCCAGAGGTCCA
TCTCGCTGAGATACGAAGGGAGGGTGTACCATTACAGGATCAACACTGCTTCTGATGGCAAGCTCTAC
GTCTCCTCCGAGAGCCGCTTCAACACCCTGGCCGAGTTGGTTCATCATCATTCAACGGTGGCCGACGG
GCTCATCACCACGCTCCATTATCCAGCCCCAAAGCGCAACAAGCCCACTGTCTATGGTGTGTCCCCCA
ACTACGACAAGTGGGAGATGGAACGCACGGACATCACCATGAAGCACAAGCTGGGCGGGGGCCAGTAC
GGGGAGGTGTACGAGGGCGTGTGGAAGAAATACAGCCTGACGGTGGCCGTGAAGACCTTGAAGGAGGA
CACCATGGAGGTGGAAGAGTTCTTGAAAGAAGCTGCAGTCATGAAAGAGATCAAACACCCTAACCTGG
TGCAGCTCCTTGGGGTCTGCACCCGGGAGCCCCCGTTCTATATCATCACTGAGTTCATGACCTACGGG
AACCTCCTGGACTACCTGAGGGAGTGCAACCGGCAGGAGGTGAACGCCGTGGTGCTGCTGTACATGGC
CACTCAGATCTCGTCAGCCATGGAGTACCTGGAGAAGAAAAACTTCATCCACAGAGATCTTGCTGCCC
GAAACTGCCTGGTAGGGGAGAACCACTTGGTGAAGGTAGCTGATTTTGGCCTGAGCAGGTTGATGACA
GGGGACACCTACACAGCCCATGCTGGAGCCAAGTTCCCCATCAAATGGACTGCACCCGAGAGCCTGGC
CTACAACAAGTTCTCCATCAAGTCCGACGTCTGGGCATTTGGAGTATTGCTTTGGGAAATTGCTACCT
ATGGCATGTCCCCTTACCCGGGAATTGACCTGTCCCAGGTGTATGAGCTGCTAGAGAAGGACTACCGC
ATGGAGCGCCCAGAAGGCTGCCCAGAGAAGGTCTATGAACTCATGCGAGCATGTTGGCAGTGGAATCC
CTCTGACCGGCCCTCCTTTGCTGAAATCCACCAAGCCTTTGAAACAATGTTCCAGGAATCCAGTATCT
CAGACGAAGTGGAAAAGGAGCTGGGGAAACAAGGCGTCCGTGGGGCTGTGAGTACCTTGCTGCAGGCC
CCAGAGCTGCCCACCAAGACGAGGACCTCCAGGAGAGCTGCAGAGCACAGAGACACCACTGACGTGCC
TGAGATGCCTCACTCCAAGGGCCAGGGAGAGAGCGATCCTCTGGACCATGAGCCTGCCGTGTCTCCAT
TGCTCCCTCGAAAAGAGCGAGGTCCCCCGGAGGGCGGCCTGAATGAAGATGAGCGCCTTCTCCCCAAA
GACAAAAAGACCAACTTGTTCAGCGCCTTGATCAAGAAGAAGAAGAAGACAGCCCCAACCCCTCCCAA
ACGCAGCAGCTCCTTCCGGGAGATGGACGGCCAGCCGGAGCGCAGAGGGGCCGGCGAGGAAGAGGGCC
GAGACATCAGCAACGGGGCACTGGCTTTCACCCCCTTGGACACAGCTGACCCAGCCAAGTCCCCAAAG
CCCAGCAATGGGGCTGGGGTCCCCAATGGAGCCCTCCGGGAGTCCGGGGGCTCAGGCTTCCGGTCTCC
CCACCTGTGGAAGAAGTCCAGCACGCTGACCAGCAGCCGCCTAGCCACCGGCGAGGAGGAGGGCGGTG
GCAGCTCCAGCAAGCGCTTCCTGCGCTCTTGCTCCGCCTCCTGCGTTCCCCATGGGGCCAAGGACACG
GAGTGGAGGTCAGTCACGCTGCCTCGGGACTTGCAGTCCACGGGAAGACAGTTTGACTCGTCCACATT
TGGAGGGCACAAAAGTGAGAAGCCGGCTCTGCCTCGGAAGAGGGCAGGGGAGAACAGGTCTGACCAGG
TGACCCGAGGCACAGTAACGCCTCCCCCCAGGCTGGTGAAAAAGAATGAGGAAGCTGCTGATGAGGTC
TTCAAAGACATCATGGAGTCCAGCCCGGGCTCCAGCCCGCCCAACCTGACTCCAAAACCCCTCCGGCG
GCAGGTCACCGTGGCCCCTGCCTCGGGCCTCCCCCACAAGGAAGAAGCTGGAAAGGGCAGTGCCTTAG
GGACCCCTGCTGCAGCTGAGCCAGTGACCCCCACCAGCAAAGCAGGCTCAGGTGCACCAGGGGGCACC
AGCAAGGGCCCCGCCGAGGAGTCCAGAGTGAGGAGGCACAAGCACTCCTCTGAGTCGCCAGGGAGGGA
CAAGGGGAAATTGTCCAGGCTCAAACCTGCCCCGCCGCCCCCACCAGCAGCCTCTGCAGGGAAGGCTG
GAGGAAAGCCCTCGCAGAGCCCGAGCCAGGAGGCGGCCGGGGAGGCAGTCCTGGGCGCAAAGACAAAA
GCCACGAGTCTGGTTGATGCTGTGAACAGTGACGCTGCCAAGCCCAGCCAGCCGGGAGAGGGCCTCAA
AAAGCCCGTGCTCCCGGCCACTCCAAAGCCACAGTCCGCCAAGCCGTCGGGGACCCCCATCAGCCCAG
CCCCCGTTCCCTCCACGTTGCCATCAGCATCCTCGGCCCTGGCAGGGGACCAGCCGTCTTCCACCGCC
TTCATCCCTCTCATATCAACCCGAGTGTCTCTTCGGAAAACCCGCCAGCCTCCAGAGCGGATCGCCAG
CGGCGCCATCACCAAGGGCGTGGTCCTGGACAGCACCGAGGCGCTGTGCCTCGCCATCTCTAGGAACT
CCGAGCAGATGGCCAGCCACAGCGCAGTGCTGGAGGCCGGCAAAAACCTCTACACGTTCTGCGTGAGC
TATGTGGATTCCATCCAGCAAATGAGGAACAAGTTTGCCTTCCGAGAGGCCATCAACAAACTGGAGAA
TAATCTCCGGGAGCTTCAGATCTGCCCGGCGACAGCAGGCAGTGGTCCAGCGGCCACTCAGGACTTCA
GCAAGCTCCTCAGTTCGGTGAAGGAAATCAGTGACATAGTGCAGAGGTAGCAGCAGTCAGGGGTCAGG
TGTCAGGCCCGTCGGAGCTGCCTGCAGCACATGCGGGCTCGCCCATACCCGTGACAGTGGCTGACAAG
GGACTAGTGAGTCAGCACCTTGGCCCAGGAGCTCTGCGCCAGGCAGAGCTGAGGGCCCTGTGGAGTCC
AGCTCTACTACCTACGTTTGCACCGCCTGCCCTCCCGCACCTTCCTCCTCCCCGCTCCGTCTCTGTCC
TCGAATTTTATCTGTGGAGTTCCTGCTCCGTGGACTGCAGTCGGCATGCCAGGACCCGCCAGCCCCGC
TCCCACCTAGTGCCCCAGACTGAGCTCTCCAGGCCAGGTGGGAACGGCTGATGTGGACTGTCTTTTTC
ATTTTTTTCTCTCTGGAGCCCCTCCTCCCCCGGCTGGGCCTCCTTCTTCCACTTCTCCAAGAATGGAA
GCCTGAACTGAGGCCTTGTGTGTCAGGCCCTCTGCCTGCACTCCCTGGCCTTGCCCGTCGTGTGCTGA
AGACATGTTTCAAGAACCGCATTTCGGGAAGGGCATGCACGGGCATGCACACGGCTGGTCACTCTGCC
CTCTGCTGCTGCCCGGGGTGGGGTGCACTCGCCATTTCCTCACGTGCAGGACAGCTCTTGATTTGGGT
GGAAAACAGGGTGCTAAAGCCAACCAGCCTTTGGGTCCTGGGCAGGTGGGAGCTGAAAAGGATCGAGG
CATGGGGCATGTCCTTTCCATCTGTCCACATCCCCAGAGCCCAGCTCTTGCTCTCTTGTGACGTGCAC
TGTGAATCCTGGCAAGAAAGCTTGAGTCTCAAGGGTGGCAGGTCACTGTCACTGCCGACATCCCTCCC
CCAGCAGAATGGAGGCAGGGGACAAGGGAGGCAGTGGCTAGTGGGGTGAACAGCTGGTGCCAAATAGC
CCCAGACTGGGCCCAGGCAGGTCTGCAAGGGCCCAGAGTGAACCGTCCTTTCACACATCTGGGTGCCC
TGAAAGGGCCCTTCCCCTCCCCCACTCCTCTAAGACAAAGTAGATTCTTACAAGGCCCTTTCCTTTGG
AACAAGACAGCCTTCACTTTTCTGAGTTCTTGAAGCATTTCAAAGCCCTGCCTCTGTGTAGCCGCCCT
GAGAGAGAATAGAGCTGCCACTGGGCACCTGCGCACAGGTGGGAGGAAAGGGCCTGGCCAGTCCTGGT
CCTGGCTGCACTCTTGAACTGGGCGAATGTCTTATTTAATTACCGTGAGTGACATAGCCTCATGTTCT
GTGGGGGTCATCAGGGAGGGTTAGGAAAACCACAAACGGAGCCCCTGAAAGCCTCACGTATTTCACAG
AGCACGCCTGCCATCTTCTCCCCGAGGCTGCCCCAGGCCGGAGCCCAGATACGGGGGCTGTGACTCTG
GGCAGGGACCCGGGGTCTCCTGGACCTTGACAGAGCAGCTAACTCCGAGAGCAGTGGGCAGGTGGCCG
CCCCTGAGGCTTCACGCCGGGAGAAGCCACCTTCCCACCCCTTCATACCGCCTCGTGCCAGCAGCCTC
GCACAGGCCCTAGCTTTACGCTCATCACCTAAACTTGTACTTTATTTTTCTGATAGAAATGGTTTCCT
CTGGATCGTTTTATGCGGTTCTTACAGCACATCACCTCTTTGCCCCCGACGGCTGTGACGCAGCCGGA
GGGAGGCACTAGTCACCGACAGCGGCCTTGAAGACAGAGCAAAGCGCCCACCCAGGTCCCCCGACTGC
CTGTCTCCATGAGGTACTGGTCCCTTCCTTTTGTTAACGTGATGTGCCACTATATTTTACACGTATCT
CTTGGTATGCATCTTTTATAGACGCTCTTTTCTAAGTGGCGTGTGCATAGCGTCCTGCCCTGCCCCCT
CGGGGGCCTGTGGTGGCTCCCCCTCTGCTTCTCGGGGTCCAGTGCATTTTGTTTCTGTATATGATTCT
CTGTGGTTTTTTTTGAATCCAAATCTGTCCTCTGTAGTATTTTTTAAATAAATCAGTGTTTACATTAG
AA
(SEQ ID NO: 79)
AATCCATCATGGCGGCCGCGGGGTTTGGTGTCTGTGCCTGAGCAGCGCTGGAGCCGGAGCCG
GTTCCCGGGTCCTGCGGCTGAGGAGCCCCTCCGCTGTCCACGGCCCCTACCGGCGGGGGGCG
GCTGGGTCCCTCGGCGGAGCTCGGGAGATGTGACTGCCTGAGGGCGGTGGTGGTGTCAGCGT
CCGGGGCCGGGGGAGGGGGTGTCTCGGGCAGAGACCCCCGGGCTTGGGGCAGCTGAGGCGGC
CGGGCCTCCTCTACACGGGGCCCGCCTTCCGCTGTCTGGGCCGCGAGAGTCCTTCGTCCCTT
ACAGCCCCGCCCCGGCTTTGGGACACTGCGGGTGGTCTGTTTCCCCCAGCTTGGGACACCCC
GTTTTCTGAGGCGTGGAAGAGCGTCGCCCCGGAGTAAGCTGCCCGTGCCGCGCCCCGACAGC
TTCCCTCAGCCCCAAGCCGCCCCTTATTCCGGATCCCGGCCCCAACTTTGGCCACGGAGCCT
CCCATTCAAATCCCTCCCTTGCTGTCAAGGGGTCTCCCCTTCCCCCAAGGTGGCTCCCGCGA
GCCTCTAATGCCCTGACTTCTTCCAATGTCACCTACGGCCCCCTTAGTCTCAGCTCAGCCAA
AAACTTTAATGCAAAGGAAAAGTCTGGATTGGTTCCACAGGCCTTTTAAAAAGCGGACTTAA
AAGTTGCTGGCAATGCATTCCTTTTCGTCAGAGTCGAGGGCAAACTCGCTGAAATCTGGGTG
ACCCGTGTCCTTTTCCGGAGAGCAAAGCAGAGAAGCGAGAGCGGCCACTAGTTCGGCAGGAA
ATTTGTTGGAAGATGAAGAAGCTAAGATAGGGGGTTGGTGACTTCCACAGGAAAAGTTCTGG
AGGAGTAGCCAAAGACCATCAGCGTTTCCTTTATGTGTGAGAATTGAAATGACTAGCATTAT
TGACCCTTTTCAGCATCCCCTGTGAATATTTCTGTTTAGGTTTTTCTTCTTGAAAAGAAATT
GTTATTCAGCCCGTTTAAAACAAATCAAGAAACTTTTGGGTAACATTGCAATTACATGAAAT
TGATAACCGCGAAAATAATTGGAACTCCTGCTTGCAAGTGTCAACCTAAAAAAAGTGCTTCC
TTTTGTTATGGAAGATGTCTTTCTGTGATTGACTTCAATTGCTGACTTGTGGAGATGCAGCG
AATGTGAAATCCCACGTATATGCCATTTCCCTCTACGCTCGCTGACCGTTCTGGAAGATCTT
GAACCCTCTTCTGGAAAGGGGTACCTATTATTACTTTATGGGGCAGCAGCCTGGAAAAGTAC
TTGGGGACCAAAGAAGGCCAAGCTTGCCTGCCCTGCATTTTATCAAAGGAGCAGGGAAGAAG
GAATCATCGAGGCATGGGGGTCCACACTGCAATGTTTTTGTGGAACATGAAGCCCTTCAGCG
GCCAGTAGCATCTGACTTTGAGCCTCAGGGTCTGAGTGAAGCCGCTCGTTGGAACTCCAAGG
AAAACCTTCTCGCTGGACCCAGTGAAAATGACCCCAACCTTTTCGTTGCACTGTATGATTTT
GTGGCCAGTGGAGATAACACTCTAAGCATAACTAAAGGTGAAAAGCTCCGGGTCTTAGGCTA
TAATCACAATGGGGAATGGTGTGAAGCCCAAACCAAAAATGGCCAAGGCTGGGTCCCAAGCA
ACTACATCACGCCAGTCAACAGTCTGGAGAAACACTCCTGGTACCATGGGCCTGTGTCCCGC
AATGCCGCTGAGTATCTGCTGAGCAGCGGGATCAATGGCAGCTTCTTGGTGCGTGAGAGTGA
GAGCAGTCCTGGCCAGAGGTCCATCTCGCTGAGATACGAAGGGAGGGTGTACCATTACAGGA
TCAACACTGCTTCTGATGGCAAGCTCTACGTCTCCTCCGAGAGCCGCTTCAACACCCTGGCC
GAGTTGGTTCATCATCATTCAACGGTGGCCGACGGGCTCATCACCACGCTCCATTATCCAGC
CCCAAAGCGCAACAAGCCCACTGTCTATGGTGTGTCCCCCAACTACGACAAGTGGGAGATGG
AACGCACGGACATCACCATGAAGCACAAGCTGGGCGGGGGCCAGTACGGGGAGGTGTACGAG
GGCGTGTGGAAGAAATACAGCCTGACGGTGGCCGTGAAGACCTTGAAGGAGGACACCATGGA
GGTGGAAGAGTTCTTGAAAGAAGCTGCAGTCATGAAAGAGATCAAACACCCTAACCTGGTGC
AGCTCCTTGGGGTCTGCACCCGGGAGCCCCCGTTCTATATCATCACTGAGTTCATGACCTAC
GGGAACCTCCTGGACTACCTGAGGGAGTGCAACCGGCAGGAGGTGAACGCCGTGGTGCTGCT
GTACATGGCCACTCAGATCTCGTCAGCCATGGAGTACCTGGAGAAGAAAAACTTCATCCACA
GAGATCTTGCTGCCCGAAACTGCCTGGTAGGGGAGAACCACTTGGTGAAGGTAGCTGATTTT
GGCCTGAGCAGGTTGATGACAGGGGACACCTACACAGCCCATGCTGGAGCCAAGTTCCCCAT
CAAATGGACTGCACCCGAGAGCCTGGCCTACAACAAGTTCTCCATCAAGTCCGACGTCTGGG
CATTTGGAGTATTGCTTTGGGAAATTGCTACCTATGGCATGTCCCCTTACCCGGGAATTGAC
CTGTCCCAGGTGTATGAGCTGCTAGAGAAGGACTACCGCATGGAGCGCCCAGAAGGCTGCCC
AGAGAAGGTCTATGAACTCATGCGAGCATGTTGGCAGTGGAATCCCTCTGACCGGCCCTCCT
TTGCTGAAATCCACCAAGCCTTTGAAACAATGTTCCAGGAATCCAGTATCTCAGACGAAGTG
GAAAAGGAGCTGGGGAAACAAGGCGTCCGTGGGGCTGTGAGTACCTTGCTGCAGGCCCCAGA
GCTGCCCACCAAGACGAGGACCTCCAGGAGAGCTGCAGAGCACAGAGACACCACTGACGTGC
CTGAGATGCCTCACTCCAAGGGCCAGGGAGAGAGCGATCCTCTGGACCATGAGCCTGCCGTG
TCTCCATTGCTCCCTCGAAAAGAGCGAGGTCCCCCGGAGGGCGGCCTGAATGAAGATGAGCG
CCTTCTCCCCAAAGACAAAAAGACCAACTTGTTCAGCGCCTTGATCAAGAAGAAGAAGAAGA
CAGCCCCAACCCCTCCCAAACGCAGCAGCTCCTTCCGGGAGATGGACGGCCAGCCGGAGCGC
AGAGGGGCCGGCGAGGAAGAGGGCCGAGACATCAGCAACGGGGCACTGGCTTTCACCCCCTT
GGACACAGCTGACCCAGCCAAGTCCCCAAAGCCCAGCAATGGGGCTGGGGTCCCCAATGGAG
CCCTCCGGGAGTCCGGGGGCTCAGGCTTCCGGTCTCCCCACCTGTGGAAGAAGTCCAGCACG
CTGACCAGCAGCCGCCTAGCCACCGGCGAGGAGGAGGGCGGTGGCAGCTCCAGCAAGCGCTT
CCTGCGCTCTTGCTCCGCCTCCTGCGTTCCCCATGGGGCCAAGGACACGGAGTGGAGGTCAG
TCACGCTGCCTCGGGACTTGCAGTCCACGGGAAGACAGTTTGACTCGTCCACATTTGGAGGG
CACAAAAGTGAGAAGCCGGCTCTGCCTCGGAAGAGGGCAGGGGAGAACAGGTCTGACCAGGT
GACCCGAGGCACAGTAACGCCTCCCCCCAGGCTGGTGAAAAAGAATGAGGAAGCTGCTGATG
AGGTCTTCAAAGACATCATGGAGTCCAGCCCGGGCTCCAGCCCGCCCAACCTGACTCCAAAA
CCCCTCCGGCGGCAGGTCACCGTGGCCCCTGCCTCGGGCCTCCCCCACAAGGAAGAAGCTGG
AAAGGGCAGTGCCTTAGGGACCCCTGCTGCAGCTGAGCCAGTGACCCCCACCAGCAAAGCAG
GCTCAGGTGCACCAGGGGGCACCAGCAAGGGCCCCGCCGAGGAGTCCAGAGTGAGGAGGCAC
AAGCACTCCTCTGAGTCGCCAGGGAGGGACAAGGGGAAATTGTCCAGGCTCAAACCTGCCCC
GCCGCCCCCACCAGCAGCCTCTGCAGGGAAGGCTGGAGGAAAGCCCTCGCAGAGCCCGAGCC
AGGAGGCGGCCGGGGAGGCAGTCCTGGGCGCAAAGACAAAAGCCACGAGTCTGGTTGATGCT
GTGAACAGTGACGCTGCCAAGCCCAGCCAGCCGGGAGAGGGCCTCAAAAAGCCCGTGCTCCC
GGCCACTCCAAAGCCACAGTCCGCCAAGCCGTCGGGGACCCCCATCAGCCCAGCCCCCGTTC
CCTCCACGTTGCCATCAGCATCCTCGGCCCTGGCAGGGGACCAGCCGTCTTCCACCGCCTTC
ATCCCTCTCATATCAACCCGAGTGTCTCTTCGGAAAACCCGCCAGCCTCCAGAGCGGATCGC
CAGCGGCGCCATCACCAAGGGCGTGGTCCTGGACAGCACCGAGGCGCTGTGCCTCGCCATCT
CTAGGAACTCCGAGCAGATGGCCAGCCACAGCGCAGTGCTGGAGGCCGGCAAAAACCTCTAC
ACGTTCTGCGTGAGCTATGTGGATTCCATCCAGCAAATGAGGAACAAGTTTGCCTTCCGAGA
GGCCATCAACAAACTGGAGAATAATCTCCGGGAGCTTCAGATCTGCCCGGCGACAGCAGGCA
GTGGTCCAGCGGCCACTCAGGACTTCAGCAAGCTCCTCAGTTCGGTGAAGGAAATCAGTGAC
ATAGTGCAGAGGTAGCAGCAGTCAGGGGTCAGGTGTCAGGCCCGTCGGAGCTGCCTGCAGCA
CATGCGGGCTCGCCCATACCCGTGACAGTGGCTGACAAGGGACTAGTGAGTCAGCACCTTGG
CCCAGGAGCTCTGCGCCAGGCAGAGCTGAGGGCCCTGTGGAGTCCAGCTCTACTACCTACGT
TTGCACCGCCTGCCCTCCCGCACCTTCCTCCTCCCCGCTCCGTCTCTGTCCTCGAATTTTAT
CTGTGGAGTTCCTGCTCCGTGGACTGCAGTCGGCATGCCAGGACCCGCCAGCCCCGCTCCCA
CCTAGTGCCCCAGACTGAGCTCTCCAGGCCAGGTGGGAACGGCTGATGTGGACTGTCTTTTT
CATTTTTTTCTCTCTGGAGCCCCTCCTCCCCCGGCTGGGCCTCCTTCTTCCACTTCTCCAAG
AATGGAAGCCTGAACTGAGGCCTTGTGTGTCAGGCCCTCTGCCTGCACTCCCTGGCCTTGCC
CGTCGTGTGCTGAAGACATGTTTCAAGAACCGCATTTCGGGAAGGGCATGCACGGGCATGCA
CACGGCTGGTCACTCTGCCCTCTGCTGCTGCCCGGGGGGGGTGCACTCGCCATTTCCTCAC
GTGCAGGACAGCTCTTGATTTGGGTGGAAAACAGGGTGCTAAAGCCAACCAGCCTTTGGGTC
CTGGGCAGGTGGGAGCTGAAAAGGATCGAGGCATGGGGCATGTCCTTTCCATCTGTCCACAT
CCCCAGAGCCCAGCTCTTGCTCTCTTGTGACGTGCACTGTGAATCCTGGCAAGAAAGCTTGA
GTCTCAAGGGTGGCAGGTCACTGTCACTGCCGACATCCCTCCCCCAGCAGAATGGAGGCAGG
GGACAAGGGAGGCAGTGGCTAGTGGGGTGAACAGCTGGTGCCAAATAGCCCCAGACTGGGCC
CAGGCAGGTCTGCAAGGGCCCAGAGTGAACCGTCCTTTCACACATCTGGGTGCCCTGAAAGG
GCCCTTCCCCTCCCCCACTCCTCTAAGACAAAGTAGATTCTTACAAGGCCCTTTCCTTTGGA
ACAAGACAGCCTTCACTTTTCTGAGTTCTTGAAGCATTTCAAAGCCCTGCCTCTGTGTAGCC
GCCCTGAGAGAGAATAGAGCTGCCACTGGGCACCTGCGCACAGGTGGGAGGAAAGGGCCTGG
CCAGTCCTGGTCCTGGCTGCACTCTTGAACTGGGCGAATGTCTTATTTAATTACCGTGAGTG
ACATAGCCTCATGTTCTGTGGGGGTCATCAGGGAGGGTTAGGAAAACCACAAACGGAGCCCC
TGAAAGCCTCACGTATTTCACAGAGCACGCCTGCCATCTTCTCCCCGAGGCTGCCCCAGGCC
GGAGCCCAGATACGGGGGCTGTGACTCTGGGCAGGGACCCGGGGTCTCCTGGACCTTGACAG
AGCAGCTAACTCCGAGAGCAGTGGGCAGGTGGCCGCCCCTGAGGCTTCACGCCGGGAGAAGC
CACCTTCCCACCCCTTCATACCGCCTCGTGCCAGCAGCCTCGCACAGGCCCTAGCTTTACGC
TCATCACCTAAACTTGTACTTTATTTTTCTGATAGAAATGGTTTCCTCTGGATCGTTTTATG
CGGTTCTTACAGCACATCACCTCTTTGCCCCCGACGGCTGTGACGCAGCCGGAGGGAGGCAC
TAGTCACCGACAGCGGCCTTGAAGACAGAGCAAAGCGCCCACCCAGGTCCCCCGACTGCCTG
TCTCCATGAGGTACTGGTCCCTTCCTTTTGTTAACGTGATGTGCCACTATATTTTACACGTA
TCTCTTGGTATGCATCTTTTATAGACGCTCTTTTCTAAGTGGCGTGTGCATAGCGTCCTGCC
CTGCCCCCTCGGGGGCCTGTGGTGGCTCCCCCTCTGCTTCTCGGGGTCCAGTGCATTTTGTT
TCTGTATATGATTCTCTGTGGTTTTTTTTGAATCCAAATCTGTCCTCTGTAGTATTTTTTAA
ATAAATCAGTGTTTACATTAGAA

Exemplary amino acid sequences of an ABL1 polypeptide are provided herein as SEQ ID NOs: 22 and 80.

(SEQ ID NO: 22)
MLEICLKLVGCKSKKGLSSSSSCYLEEALQRPVASDFEPQGLSEAARWNSKENLLAGPSENDPNLFVA
LYDFVASGDNTLSITKGEKLRVLGYNHNGEWCEAQTKNGQGWVPSNYITPVNSLEKHSWYHGPVSRNA
AEYLLSSGINGSFLVRESESSPGQRSISLRYEGRVYHYRINTASDGKLYVSSESRFNTLAELVHHHST
VADGLITTLHYPAPKRNKPTVYGVSPNYDKWEMERTDITMKHKLGGGQYGEVYEGVWKKYSLTVAVKT
LKEDTMEVEEFLKEAAVMKEIKHPNLVQLLGVCTREPPFYIITEFMTYGNLLDYLRECNRQEVNAVVL
LYMATQISSAMEYLEKKNFIHRDLAARNCLVGENHLVKVADFGLSRLMTGDTYTAHAGAKFPIKWTAP
ESLAYNKFSIKSDVWAFGVLLWEIATYGMSPYPGIDLSQVYELLEKDYRMERPEGCPEKVYELMRACW
QWNPSDRPSFAEIHQAFETMFQESSISDEVEKELGKQGVRGAVSTLLQAPELPTKTRTSRRAAEHRDT
TDVPEMPHSKGQGESDPLDHEPAVSPLLPRKERGPPEGGLNEDERLLPKDKKTNLFSALIKKKKKTAP
TPPKRSSSFREMDGQPERRGAGEEEGRDISNGALAFTPLDTADPAKSPKPSNGAGVPNGALRESGGSG
FRSPHLWKKSSTLTSSRLATGEEEGGGSSSKRFLRSCSASCVPHGAKDTEWRSVTLPRDLQSTGRQFD
SSTFGGHKSEKPALPRKRAGENRSDQVTRGTVTPPPRLVKKNEEAADEVFKDIMESSPGSSPPNLTPK
PLRRQVTVAPASGLPHKEEAGKGSALGTPAAAEPVTPTSKAGSGAPGGTSKGPAEESRVRRHKHSSES
PGRDKGKLSRLKPAPPPPPAASAGKAGGKPSQSPSQEAAGEAVLGAKTKATSLVDAVNSDAAKPSQPG
EGLKKPVLPATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNKFAFREAIN
KLENNLRELQICPATAGSGPAATQDFSKLLSSVKEISDIVQR
(SEQ ID NO: 80)
MGQQPGKVLGDQRRPSLPALHFIKGAGKKESSRHGGPHCNVFVEHEALQRPVASDFEPQGLS
EAARWNSKENLLAGPSENDPNLFVALYDFVASGDNTLSITKGEKLRVLGYNHNGEWCEAQTK
NGQGWVPSNYITPVNSLEKHSWYHGPVSRNAAEYLLSSGINGSFLVRESESSPGQRSISLRY
EGRVYHYRINTASDGKLYVSSESRFNTLAELVHHHSTVADGLITTLHYPAPKRNKPTVYGVS
PNYDKWEMERTDITMKHKLGGGQYGEVYEGVWKKYSLTVAVKTLKEDTMEVEEFLKEAAVMK
EIKHPNLVQLLGVCTREPPFYIITEFMTYGNLLDYLRECNRQEVNAVVLLYMATQISSAMEY
LEKKNFIHRDLAARNCLVGENHLVKVADFGLSRLMTGDTYTAHAGAKFPIKWTAPESLAYNK
FSIKSDVWAFGVLLWEIATYGMSPYPGIDLSQVYELLEKDYRMERPEGCPEKVYELMRACWQ
WNPSDRPSFAEIHQAFETMFQESSISDEVEKELGKQGVRGAVSTLLQAPELPTKTRTSRRAA
EHRDTTDVPEMPHSKGQGESDPLDHEPAVSPLLPRKERGPPEGGLNEDERLLPKDKKTNLFS
ALIKKKKKTAPTPPKRSSSFREMDGQPERRGAGEEEGRDISNGALAFTPLDTADPAKSPKPS
NGAGVPNGALRESGGSGFRSPHLWKKSSTLTSSRLATGEEEGGGSSSKRFLRSCSASCVPHG
AKDTEWRSVTLPRDLQSTGRQFDSSTFGGHKSEKPALPRKRAGENRSDQVTRGTVTPPPRLV
KKNEEAADEVFKDIMESSPGSSPPNLTPKPLRRQVTVAPASGLPHKEEAGKGSALGTPAAAE
PVTPTSKAGSGAPGGTSKGPAEESRVRRHKHSSESPGRDKGKLSRLKPAPPPPPAASAGKAG
GKPSQSPSQEAAGEAVLGAKTKATSLVDAVNSDAAKPSQPGEGLKKPVLPATPKPQSAKPSG
TPISPAPVPSTLPSASSALAGDQPSSTAFIPLISTRVSLRKTRQPPERIASGAITKGVVLDS
TEALCLAISRNSEQMASHSAVLEAGKNLYTFCVSYVDSIQQMRNKFAFREAINKLENNLREL
QICPATAGSGPAATQDFSKLLSSVKEISDIVQR

### (i) ABL1 Fusion Nucleic Acid Molecules

In some embodiments, an ABL1 rearrangement results in a gene fusion, resulting in a fusion nucleic acid molecule comprising at least a portion of an ABL1 gene, and at least a portion of another gene. Accordingly, in some aspects, provided herein are ABL1 fusion nucleic acid molecules comprising at least a portion of an ABL1 gene and at least a portion of another gene.

### Exemplary ABL1 Gene Fusion Partners

In some embodiments, an ABL1 fusion nucleic acid molecule of the disclosure comprises at least a portion of an ABL1 gene and at least a portion of a BMP2K, EHMT1, MED27, RFFL, SLC27A4, BCR, NUP214, AIF1L, RCSD1, APOOL, DNAH2, RHEX/C1orf186, RALGPS1, TSC1, TSPAN18, ZNF804B, EXOSC2, or SNX2 gene. In some embodiments, an ABL1 fusion nucleic acid molecule of the disclosure comprises at least a portion of an ABL1 gene and at least a portion of a fusion partner gene as provided in Table 1 or Table 2 of Example 1 herein.

As used herein “BMP2K” refers to a gene encoding an BMP2K mRNA or polypeptide. The BMP2K gene encodes the BMP-2-inducible protein kinase. BMP2K is also known as BIKE and HRIHFB2017. In some embodiments, a BMP2K gene according to the present disclosure is a human BMP2K gene. An exemplary BMP2K gene is represented by NCBI Gene ID No. 55589. An exemplary BMP2K mRNA sequence is represented by NCBI Ref. Seq. NM_198892 and provided herein as SEQ ID NO: 43. An exemplary amino acid sequence of a BMP2K polypeptide is provided herein as SEQ ID NO: 44.

As used herein “EHMT1” refers to a gene encoding an EHMT1 mRNA or polypeptide. The EHMT1 gene encodes a histone-lysine N-methyltransferase. EHMT1 is also known as EHMT1-IT1, Eu-HMTase1, EUHMTASE1, FP13812, GLP, GLP1, KLEFS1 and KMTID. In some embodiments, an EHMT1 gene according to the present disclosure is a human EHMT1 gene. An exemplary EHMT1 gene is represented by NCBI Gene ID No. 79813. An exemplary EHMT1 mRNA sequence is represented by NCBI Ref. Seq. NM_024757 and provided herein as SEQ ID NO: 45. An exemplary amino acid sequence of an EHMT1 polypeptide is provided herein as SEQ ID NO: 46.

As used herein “MED27” refers to a gene encoding a MED27 mRNA or polypeptide. The MED27 gene encodes the mediator of RNA polymerase II transcription subunit 27. MED27 is also known as CRAP34, CRSP34, CRSP8, MED3, NEDSCAC, and TRAP37. In some embodiments, a MED27 gene according to the present disclosure is a human MED27 gene. An exemplary MED27 gene is represented by NCBI Gene ID No. 9442. An exemplary MED27 mRNA sequence is represented by NCBI Ref. Seq. NM_004269 and provided herein as SEQ ID NO: 47. An exemplary amino acid sequence of a MED27 polypeptide is provided herein as SEQ ID NO: 48.

As used herein “RFFL” refers to a gene encoding an RFFL mRNA or polypeptide. The RFFL gene encodes the E3 ubiquitin-protein ligase rififylin. RFFL is also known as CARP-2, CARP2, FRING, RIFIFYLIN, RNF189, and RNF34L. In some embodiments, a RFFL gene according to the present disclosure is a human RFFL gene. An exemplary RFFL gene is represented by NCBI Gene ID No. 117584. An exemplary RFFL mRNA sequence is represented by NCBI Ref. Seq. NM_001017368 and provided herein as SEQ ID NO: 49. An exemplary amino acid sequence of a RFFL polypeptide is provided herein as SEQ ID NO: 50.

As used herein “SLC27A4” refers to a gene encoding an SLC27A4 mRNA or polypeptide. The SLC27A4 gene encodes the long-chain fatty acid transport protein 4. SLC27A4 is also known as ACSVL4, FATP4, and IPS. In some embodiments, a SLC27A4 gene according to the present disclosure is a human SLC27A4 gene. An exemplary SLC27A4 gene is represented by NCBI Gene ID No. 10999. An exemplary SLC27A4 mRNA sequence is represented by NCBI Ref. Seq. NM_005094 and provided herein as SEQ ID NO: 51. An exemplary amino acid sequence of a SLC27A4 polypeptide is provided herein as SEQ ID NO: 52.

As used herein “BCR” refers to a gene encoding a BCR mRNA or polypeptide. The BCR gene encodes the breakpoint cluster region protein. BCR is also known as ALL, BCR1, CML, D22S11, D22S662, and PHL. In some embodiments, a BCR gene according to the present disclosure is a human BCR gene. An exemplary BCR gene is represented by NCBI Gene ID No. 613. An exemplary BCR mRNA sequence is represented by NCBI Ref. Seq. NM_004327 and provided herein as SEQ ID NO: 53. An exemplary amino acid sequence of a BCR polypeptide is provided herein as SEQ ID NO: 54.

As used herein “NUP214” refers to a gene encoding a NUP214 mRNA or polypeptide. The NUP214 gene encodes a nuclear pore complex protein. NUP214 is also known as CAIN, CAN, and IIAE9. In some embodiments, a NUP214 gene according to the present disclosure is a human NUP214 gene. An exemplary NUP214 gene is represented by NCBI Gene ID No. 8021. An exemplary NUP214 mRNA sequence is represented by NCBI Ref. Seq. NM_005085 and provided herein as SEQ ID NO: 55. An exemplary amino acid sequence of a NUP214 polypeptide is provided herein as SEQ ID NO: 56.

As used herein “AIF1L” refers to a gene encoding an AIF1L mRNA or polypeptide. The AIF1L gene encodes the allograft inflammatory factor 1-like protein. AIF1L is also known as C9orf58 and IBA2. In some embodiments, an AIF1L gene according to the present disclosure is a human AIF1L gene. An exemplary AIF1L gene is represented by NCBI Gene ID No. 83543. An exemplary AIF1L mRNA sequence is represented by NCBI Ref. Seq. NM_031426 and provided herein as SEQ ID NO: 57. An exemplary amino acid sequence of an AIF1L polypeptide is provided herein as SEQ ID NO: 58.

As used herein “RCSD1” refers to a gene encoding an RCSD1 mRNA or polypeptide. The RCSD1 gene encodes the capZ-interacting protein. RCSD1 is also known as CAPZIP and MK2S4. In some embodiments, a RCSD1 gene according to the present disclosure is a human RCSD1 gene. An exemplary RCSD1 gene is represented by NCBI Gene ID No. 92241. An exemplary RCSD1 mRNA sequence is represented by NCBI Ref. Seq. NM_052862 and provided herein as SEQ ID NO: 59. An exemplary amino acid sequence of a RCSD1 polypeptide is provided herein as SEQ ID NO: 60.

As used herein “APOOL” refers to a gene encoding an APOOL mRNA or polypeptide. The APOOL gene encodes a MICOS complex subunit. APOOL is also known as CXorf33, FAM121A, Mic27, MICOS27, and UNQ8193. In some embodiments, an APOOL gene according to the present disclosure is a human APOOL gene. An exemplary APOOL gene is represented by NCBI Gene ID No. 139322. An exemplary APOOL mRNA sequence is represented by NCBI Ref. Seq. NM_198450 and provided herein as SEQ ID NO: 61. An exemplary amino acid sequence of an APOOL polypeptide is provided herein as SEQ ID NO: 62.

As used herein “DNAH2” refers to a gene encoding a DNAH2 mRNA or polypeptide. The DNAH2 gene encodes a dynein axonemal heavy chain 2 protein. DNAH2 is also known as DNAHC2, DNHD3 and SPGF45. In some embodiments, a DNAH2 gene according to the present disclosure is a human DNAH2 gene. An exemplary DNAH2 gene is represented by NCBI Gene ID No. 146754. An exemplary DNAH2 mRNA sequence is represented by NCBI Ref. Seq. NM_020877 and provided herein as SEQ ID NO: 63. An exemplary amino acid sequence of a DNAH2 polypeptide is provided herein as SEQ ID NO: 64.

As used herein “RHEX/C1orf186” refers to a gene encoding a RHEX/C1orf186 mRNA or polypeptide. The RHEX/C1orf186 gene encodes the regulator of hemoglobinization and erythroid cell expansion protein. RHEX/C1orf186 is also known as RHEX, and Clorf186. In some embodiments, a RHEX/C1orf186 gene according to the present disclosure is a human RHEX/C1orf186 gene. An exemplary RHEX/C1orf186 gene is represented by NCBI Gene ID No. 440712. An exemplary RHEX/Clorf186 mRNA sequence is represented by NCBI Ref. Seq. NM_001007544 and provided herein as SEQ ID NO: 65. An exemplary amino acid sequence of a RHEX/C1orf186 polypeptide is provided herein as SEQ ID NO: 66.

As used herein “RALGPS1” refers to a gene encoding a RALGPS1 mRNA or polypeptide. The RALGPS1 gene encodes a ras-specific guanine nucleotide-releasing factor. RALGPS1 is also known as RALGEF2 and RALGPS1A. In some embodiments, a RALGPS1 gene according to the present disclosure is a human RALGPS1 gene. An exemplary RALGPS1 gene is represented by NCBI Gene ID No. 9649. An exemplary RALGPS1 mRNA sequence is represented by NCBI Ref. Seq. NM_014636 and provided herein as SEQ ID NO:67. An exemplary amino acid sequence of a RALGPS1 polypeptide is provided herein as SEQ ID NO:68.

As used herein “TSC1” refers to a gene encoding a TSC1 mRNA or polypeptide. The TSC1 gene encodes the hamartin protein. TSC1 is also known as LAM and TSC. In some embodiments, a TSC1 gene according to the present disclosure is a human TSC1 gene. An exemplary TSC1 gene is represented by NCBI Gene ID No. 7248. An exemplary TSC1 mRNA sequence is represented by NCBI Ref. Seq. NM_000368 and provided herein as SEQ ID NO: 69. An exemplary amino acid sequence of a TSC1 polypeptide is provided herein as SEQ ID NO: 70.

As used herein “TSPAN18” refers to a gene encoding a TSPAN18 mRNA or polypeptide. The TSPAN18 gene encodes the tetraspanin-18 protein. TSPAN18 is also known as TSPAN. In some embodiments, a TSPAN gene according to the present disclosure is a human TSPAN gene. An exemplary TSPAN gene is represented by NCBI Gene ID No. 90139. An exemplary TSPAN18 mRNA sequence is represented by NCBI Ref. Seq. NM_130783 and provided herein as SEQ ID NO: 71. An exemplary amino acid sequence of a TSPAN18 polypeptide is provided herein as SEQ ID NO: 72.

As used herein “ZNF804B” refers to a gene encoding a ZNF804B mRNA or polypeptide. The ZNF804B gene encodes the zinc finger protein 804B. In some embodiments, a ZNF804B gene according to the present disclosure is a human ZNF804B gene. An exemplary ZNF804B gene is represented by NCBI Gene ID No. 219578. An exemplary ZNF804B mRNA sequence is represented by NCBI Ref. Seq. NM_181646 and provided herein as SEQ ID NO: 73. An exemplary amino acid sequence of a ZNF804B polypeptide is provided herein as SEQ ID NO: 74.

As used herein “EXOSC2” refers to a gene encoding an EXOSC2 mRNA or polypeptide. The EXOSC2 gene encodes the exosome complex component RRP4. EXOSC2 is also known as hRrp4p, p7, RRP4, Rrp4p and SHRF. In some embodiments, a EXOSC2 gene according to the present disclosure is a human EXOSC2 gene. An exemplary EXOSC2 gene is represented by NCBI Gene ID No. 23404. An exemplary EXOSC2 mRNA sequence is represented by NCBI Ref. Seq. NM_014285 and provided herein as SEQ ID NO: 75. An exemplary amino acid sequence of an EXOSC2 polypeptide is provided herein as SEQ ID NO: 76.

As used herein “SNX2” refers to a gene encoding an SNX2 mRNA or polypeptide. The SNX2 gene encodes the sorting nexin-2 protein. SNX2 is also known as TRG-9. In some embodiments, a TRG-9 gene according to the present disclosure is a human TRG-9 gene. An exemplary TRG-9 gene is represented by NCBI Gene ID No. 6643. An exemplary SNX2 mRNA sequence is represented by NCBI Ref. Seq. NM_003100 and provided herein as SEQ ID NO: 77. An exemplary amino acid sequence of a SNX2 polypeptide is provided herein as SEQ ID NO: 78.

In some embodiments, an ABL1 fusion nucleic acid molecule of the disclosure is an EHMT1-ABL1 fusion nucleic acid molecule, comprising, in 5′ to 3′ direction, at least a portion of an EHMT1 gene fused to at least a portion of an ABL1 gene. In some embodiments, an ABL1 fusion nucleic acid molecule of the disclosure is a MED27-ABL1 fusion nucleic acid molecule, comprising, in 5′ to 3′ direction, at least a portion of a MED27 gene fused to at least a portion of an ABL1 gene. In some embodiments, an ABL1 fusion nucleic acid molecule of the disclosure is an RFFL-ABL1 fusion nucleic acid molecule, comprising, in 5′ to 3′ direction, at least a portion of an RFFL gene fused to at least a portion of an ABL1 gene. In some embodiments, an ABL1 fusion nucleic acid molecule of the disclosure is an SLC27A4-ABL1 fusion nucleic acid molecule, comprising, in 5′ to 3′ direction, at least a portion of an SLC27A4 gene fused to at least a portion of an ABL1 gene. In some embodiments, an ABL1 fusion nucleic acid molecule of the disclosure is a BCR-ABL1 fusion nucleic acid molecule, comprising, in 5′ to 3′ direction, at least a portion of a BCR gene fused to at least a portion of an ABL1 gene. In some embodiments, an ABL1 fusion nucleic acid molecule of the disclosure is a NUP214-ABL1 fusion nucleic acid molecule, comprising, in 5′ to 3′ direction, at least a portion of a NUP214 gene fused to at least a portion of an ABL1 gene. In some embodiments, an ABL1 fusion nucleic acid molecule of the disclosure is an ABL1-AIF1L fusion nucleic acid molecule, comprising, in 5′ to 3′ direction, at least a portion of an ABL1 gene fused to at least a portion of an AIF1L gene. In some embodiments, an ABL1 fusion nucleic acid molecule of the disclosure is an RCSD1-ABL1 fusion nucleic acid molecule, comprising, in 5′ to 3′ direction, at least a portion of an RCSD1 gene fused to at least a portion of an ABL1 gene. In some embodiments, an ABL1 fusion nucleic acid molecule of the disclosure is an APOOL-ABL1 fusion nucleic acid molecule, comprising, in 5′ to 3′ direction, at least a portion of an APOOL gene fused to at least a portion of an ABL1 gene. In some embodiments, an ABL1 fusion nucleic acid molecule of the disclosure is an ABL1-DNAH2 fusion nucleic acid molecule, comprising, in 5′ to 3′ direction, at least a portion of an ABL1 gene fused to at least a portion of a DNAH2 gene. In some embodiments, an ABL1 fusion nucleic acid molecule of the disclosure is a RHEX/C1orf186-ABL1 fusion nucleic acid molecule, comprising, in 5′ to 3′ direction, at least a portion of a RHEX/C1orf186-gene fused to at least a portion of an ABL1 gene. In some embodiments, an ABL1 fusion nucleic acid molecule of the disclosure is a RALGPS1-ABL1 fusion nucleic acid molecule, comprising, in 5′ to 3′ direction, at least a portion of a RALGPS1 gene fused to at least a portion of an ABL1 gene. In some embodiments, an ABL1 fusion nucleic acid molecule of the disclosure is a TSC1-ABL1 fusion nucleic acid molecule, comprising, in 5′ to 3′ direction, at least a portion of a TSC1 gene fused to at least a portion of an ABL1 gene. In some embodiments, an ABL1 fusion nucleic acid molecule of the disclosure is a TSPAN18-ABL1 fusion nucleic acid molecule, comprising, in 5′ to 3′ direction, at least a portion of a TSPAN18 gene fused to at least a portion of an ABL1 gene. In some embodiments, an ABL1 fusion nucleic acid molecule of the disclosure is a ZNF804B-ABL1 fusion nucleic acid molecule, comprising, in 5′ to 3′ direction, at least a portion of a ZNF804B gene fused to at least a portion of an ABL1 gene. In some embodiments, an ABL1 fusion nucleic acid molecule of the disclosure is an ABL1-NUP214 fusion nucleic acid molecule, comprising, in 5′ to 3′ direction, at least a portion of an ABL1 gene fused to at least a portion of a NUP214 gene. In some embodiments, an ABL1 fusion nucleic acid molecule of the disclosure is an AIF1L-ABL1 fusion nucleic acid molecule, comprising, in 5′ to 3′ direction, at least a portion of an AIF1L gene fused to at least a portion of an ABL1 gene. In some embodiments, an ABL1 fusion nucleic acid molecule of the disclosure is an EXOSC2-ABL1 fusion nucleic acid molecule, comprising, in 5′ to 3′ direction, at least a portion of an EXOSC2 gene fused to at least a portion of an ABL1 gene. In some embodiments, an ABL1 fusion nucleic acid molecule of the disclosure is an SNX2-ABL1 fusion nucleic acid molecule, comprising, in 5′ to 3′ direction, at least a portion of an SNX2 gene fused to at least a portion of an ABL1 gene. In some embodiments, an ABL1 fusion nucleic acid molecule of the disclosure is a BMP2K-ABL1 fusion nucleic acid molecule, comprising, in 5′ to 3′ direction, at least a portion of a BMP2K gene fused to at least a portion of an ABL1 gene.

### Exemplary ABL1 Fusion Breakpoints

In some embodiments, an ABL1 fusion nucleic acid molecule of the disclosure is an EHMT1-ABL1 fusion nucleic acid molecule comprising or resulting from a breakpoint within intron 4 of an EHMT1 gene and/or a breakpoint within intron 1 of an ABL1 gene. In some embodiments, the EHMT1-ABL1 fusion nucleic acid molecule, comprises, in 5′ to 3′ direction, at least a portion of the EHMT1 gene fused to at least a portion of the ABL1 gene. In some embodiments, an ABL1 fusion nucleic acid molecule of the disclosure is a MED27-ABL1 fusion nucleic acid molecule comprising or resulting from a breakpoint within intron 4 of a MED27 gene and/or a breakpoint within intron 4 of an ABL1 gene. In some embodiments, the MED27-ABL1 fusion nucleic acid molecule, comprises, in the 5′ to 3′ direction, at least a portion of the MED27 gene fused to at least a portion of the ABL1 gene. In some embodiments, an ABL1 fusion nucleic acid molecule of the disclosure is an SLC27A4-ABL1 fusion nucleic acid molecule comprising or resulting from a breakpoint within intron 12 of an SLC27A4 gene and/or a breakpoint within intron 5 of an ABL1 gene. In some embodiments, the SLC27A4-ABL1 fusion nucleic acid molecule, comprises, in 5′ to 3′ direction, at least a portion of the SLC27A4 gene fused to at least a portion of the ABL1 gene. In some embodiments, an ABL1 fusion nucleic acid molecule of the disclosure is a BCR-ABL1 fusion nucleic acid molecule comprising or resulting from a breakpoint within intron 13 of a BCR gene and/or a breakpoint within intron 1 of an ABL1 gene. In some embodiments, the BCR-ABL1 fusion nucleic acid molecule, comprises, in 5′ to 3′ direction, at least a portion of the BCR gene fused to at least a portion of the ABL1 gene. In some embodiments, an ABL1 fusion nucleic acid molecule of the disclosure is a BCR-ABL1 fusion nucleic acid molecule comprising or resulting from a breakpoint within intron 14 of a BCR gene and/or a breakpoint within intron 1 of an ABL1 gene. In some embodiments, the BCR-ABL1 fusion nucleic acid molecule, comprises, in 5′ to 3′ direction, at least a portion of the BCR gene fused to at least a portion of the ABL1 gene. In some embodiments, an ABL1 fusion nucleic acid molecule of the disclosure is a BCR-ABL1 fusion nucleic acid molecule comprising or resulting from a breakpoint within intron 12 of a BCR gene and/or a breakpoint within intron 1 of an ABL1 gene. In some embodiments, the BCR-ABL1 fusion nucleic acid molecule, comprises, in 5′ to 3′ direction, at least a portion of the BCR gene fused to at least a portion of the ABL1 gene. In some embodiments, an ABL1 fusion nucleic acid molecule of the disclosure is an APOOL-ABL1 fusion nucleic acid molecule comprising or resulting from a breakpoint within intron 2 of an APOOL gene and/or a breakpoint within exon 2 of an ABL1 gene. In some embodiments, the APOOL-ABL1 fusion nucleic acid molecule, comprises, in 5′ to 3′ direction, at least a portion of the APOOL gene fused to at least a portion of the ABL1 gene. In some embodiments, an ABL1 fusion nucleic acid molecule of the disclosure is a RALGPS1-ABL1 fusion nucleic acid molecule comprising or resulting from a breakpoint within intron 1 of a RALGPS1 gene and/or a breakpoint within intron 2 of an ABL1 gene. In some embodiments, the RALGPS1-ABL1 fusion nucleic acid molecule, comprises, in 5′ to 3′ direction, at least a portion of the RALGPS1 gene fused to at least a portion of the ABL1 gene. In some embodiments, an ABL1 fusion nucleic acid molecule of the disclosure is a ZNF804B-ABL1 fusion nucleic acid molecule comprising or resulting from a breakpoint within intron 1 of a ZNF804B gene and/or a breakpoint within intron 1 of an ABL1 gene. In some embodiments, the ZNF804B-ABL1 fusion nucleic acid molecule, comprises, in 5′ to 3′ direction, at least a portion of the ZNF804B gene fused to at least a portion of the ABL1 gene. In some embodiments, an ABL1 fusion nucleic acid molecule of the disclosure is an AIF1L-ABL1 fusion nucleic acid molecule comprising or resulting from a breakpoint within intron 2 of an AIF1L gene and/or a breakpoint within intron 1 of an ABL1 gene. In some embodiments, the AIF1L-ABL1 fusion nucleic acid molecule, comprises, in the 5′ to 3′ direction, at least a portion of the AIF1L gene fused to at least a portion of the ABL1 gene. In some embodiments, an ABL1 fusion nucleic acid molecule of the disclosure is an EXOSC2-ABL1 fusion nucleic acid molecule comprising or resulting from a breakpoint within exon 9 of an EXOSC2 gene and/or a breakpoint within exon 1 of an ABL1 gene. In some embodiments, the EXOSC2-ABL1 fusion nucleic acid molecule, comprises, in 5′ to 3′ direction, at least a portion of the EXOSC2 gene fused to at least a portion of the ABL1 gene. In some embodiments, an ABL1 fusion nucleic acid molecule of the disclosure is a NUP214-ABL1 fusion nucleic acid molecule comprising or resulting from a breakpoint within intron 34 of a NUP214 gene and/or a breakpoint within exon 3 of an ABL1 gene. In some embodiments, the NUP214-ABL1 fusion nucleic acid molecule, comprises, in 5′ to 3′ direction, at least a portion of the NUP214 gene fused to at least a portion of the ABL1 gene.

In some embodiments, an ABL1 fusion nucleic acid molecule of the disclosure is an EHMT1-ABL1 fusion nucleic acid molecule, wherein the EHMT1-ABL1 fusion nucleic acid molecule comprises or results from a 5′ breakpoint within chromosomal coordinates chr9: 140630475-140630606 and/or a 3′ breakpoint within chromosomal coordinates chr9: 133729408-133729648. In some embodiments, an ABL1 fusion nucleic acid molecule of the disclosure is a MED27-ABL1 fusion nucleic acid molecule, wherein the MED27-ABL1 fusion nucleic acid molecule comprises or results from a 5′ breakpoint within chromosomal coordinates chr9: 134774423-134776006 and/or a 3′ breakpoint within chromosomal coordinates chr9: 133747364-133747762. In some embodiments, an ABL1 fusion nucleic acid molecule of the disclosure is a RFFL-ABL1 fusion nucleic acid molecule, wherein the RFFL-ABL1 fusion nucleic acid molecule comprises or results from a 5′ breakpoint within chromosomal coordinates chr17: 33343379-33343419 and/or a 3′ breakpoint within chromosomal coordinates chr9: 133729485-133729525. In some embodiments, an ABL1 fusion nucleic acid molecule of the disclosure is a SLC27A4-ABL1 fusion nucleic acid molecule, wherein the SLC27A4-ABL1 fusion nucleic acid molecule comprises or results from a 5′ breakpoint within chromosomal coordinates chr9: 131119603-131119801 and/or a 3′ breakpoint within chromosomal coordinates chr9: 133748211-133748501. In some embodiments, an ABL1 fusion nucleic acid molecule of the disclosure is a BCR-ABL1 fusion nucleic acid molecule, wherein the BCR-ABL1 fusion nucleic acid molecule comprises or results from a 5′ breakpoint within chromosomal coordinates chr22: 23632033-23632321 and/or a 3′ breakpoint within chromosomal coordinates chr9: 133591666-133591902. In some embodiments, an ABL1 fusion nucleic acid molecule of the disclosure is a BCR-ABL1 fusion nucleic acid molecule, wherein the BCR-ABL1 fusion nucleic acid molecule comprises or results from a 5′ breakpoint within chromosomal coordinates chr22: 23634537-23634672 and/or a 3′ breakpoint within chromosomal coordinates chr9: 133672815-133673059. In some embodiments, an ABL1 fusion nucleic acid molecule of the disclosure is a BCR-ABL1 fusion nucleic acid molecule, wherein the BCR-ABL1 fusion nucleic acid molecule comprises or results from a 5′ breakpoint within chromosomal coordinates chr22: 23634041-23634323 and/or a 3′ breakpoint within chromosomal coordinates chr9: 133708182-133708567. In some embodiments, an ABL1 fusion nucleic acid molecule of the disclosure is a BCR-ABL1 fusion nucleic acid molecule, wherein the BCR-ABL1 fusion nucleic acid molecule comprises or results from a 5′ breakpoint within chromosomal coordinates chr22: 23632683-23632948 and/or a 3′ breakpoint within chromosomal coordinates chr9: 133613427-133613626. In some embodiments, an ABL1 fusion nucleic acid molecule of the disclosure is a BCR-ABL1 fusion nucleic acid molecule, wherein the BCR-ABL1 fusion nucleic acid molecule comprises or results from a 5′ breakpoint within chromosomal coordinates chr22: 23634011-23634460 and/or a 3′ breakpoint within chromosomal coordinates chr9: 133645693-133646017. In some embodiments, an ABL1 fusion nucleic acid molecule of the disclosure is a BCR-ABL1 fusion nucleic acid molecule, wherein the BCR-ABL1 fusion nucleic acid molecule comprises or results from a 5′ breakpoint within chromosomal coordinates chr22: 23634211-23634632 and/or a 3′ breakpoint within chromosomal coordinates chr9: 133619101-133619395. In some embodiments, an ABL1 fusion nucleic acid molecule of the disclosure is a BCR-ABL1 fusion nucleic acid molecule, wherein the BCR-ABL1 fusion nucleic acid molecule comprises or results from a 5′ breakpoint within chromosomal coordinates chr22: 23631722-23631762 and/or a 3′ breakpoint within chromosomal coordinates chr9: 133729484-133729524. In some embodiments, an ABL1 fusion nucleic acid molecule of the disclosure is a BCR-ABL1 fusion nucleic acid molecule, wherein the BCR-ABL1 fusion nucleic acid molecule comprises or results from a 5′ breakpoint within chromosomal coordinates chr22: 23633346-23634163 and/or a 3′ breakpoint within chromosomal coordinates chr9: 133609201-133609909. In some embodiments, an ABL1 fusion nucleic acid molecule of the disclosure is a BCR-ABL1 fusion nucleic acid molecule, wherein the BCR-ABL1 fusion nucleic acid molecule comprises or results from a 5′ breakpoint within chromosomal coordinates chr22: 23633432-23633796 and/or a 3′ breakpoint within chromosomal coordinates chr9: 133623903-133624461. In some embodiments, an ABL1 fusion nucleic acid molecule of the disclosure is a BCR-ABL1 fusion nucleic acid molecule, wherein the BCR-ABL1 fusion nucleic acid molecule comprises or results from a 5′ breakpoint within chromosomal coordinates chr22: 23633837-23634208 and/or a 3′ breakpoint within chromosomal coordinates chr9: 133665426-133665727. In some embodiments, an ABL1 fusion nucleic acid molecule of the disclosure is a BCR-ABL1 fusion nucleic acid molecule, wherein the BCR-ABL1 fusion nucleic acid molecule comprises or results from a 5′ breakpoint within chromosomal coordinates chr22: 23632858-23633068 and/or a 3′ breakpoint within chromosomal coordinates chr9: 133701595-133701846. In some embodiments, an ABL1 fusion nucleic acid molecule of the disclosure is a BCR-ABL1 fusion nucleic acid molecule, wherein the BCR-ABL1 fusion nucleic acid molecule comprises or results from a 5′ breakpoint within chromosomal coordinates chr22: 23633208-23633695 and/or a 3′ breakpoint within chromosomal coordinates chr9: 133702617-133702992. In some embodiments, an ABL1 fusion nucleic acid molecule of the disclosure is a NUP214-ABL1 fusion nucleic acid molecule, wherein the NUP214-ABL1 fusion nucleic acid molecule comprises or results from a 5′ breakpoint within chromosomal coordinates chr9: 134106039-134106079 and/or a 3′ breakpoint within chromosomal coordinates chr9: 133730222-133730262. In some embodiments, an ABL1 fusion nucleic acid molecule of the disclosure is an ABL1-AIF1L fusion nucleic acid molecule, wherein the ABL1-AIF1L fusion nucleic acid molecule comprises or results from a 5′ breakpoint within chromosomal coordinates chr9: 133755952-133755992 and/or a 3′ breakpoint within chromosomal coordinates chr9: 133989965-133990005. In some embodiments, an ABL1 fusion nucleic acid molecule of the disclosure is a RCSD1-ABL1 fusion nucleic acid molecule, wherein the RCSD1-ABL1 fusion nucleic acid molecule comprises or results from a 5′ breakpoint within chromosomal coordinates chr1: 167654668-167654708 and/or a 3′ breakpoint within chromosomal coordinates chr9: 133738173-133738213. In some embodiments, an ABL1 fusion nucleic acid molecule of the disclosure is an APOOL-ABL1 fusion nucleic acid molecule, wherein the APOOL-ABL1 fusion nucleic acid molecule comprises or results from a 5′ breakpoint within chromosomal coordinates chrX: 84302705-84302904 and/or a 3′ breakpoint within chromosomal coordinates chr9: 133729584-133729766. In some embodiments, an ABL1 fusion nucleic acid molecule of the disclosure is an ABL1-DNAH2 fusion nucleic acid molecule, wherein the ABL1-DNAH2 fusion nucleic acid molecule comprises or results from a 5′ breakpoint within chromosomal coordinates chr9: 133755953-133755993 and/or a 3′ breakpoint within chromosomal coordinates chr17: 7636399-7636439. In some embodiments, an ABL1 fusion nucleic acid molecule of the disclosure is a RHEX/C1orf186-ABL1 fusion nucleic acid molecule, wherein the RHEX/C1orf186-ABL1 fusion nucleic acid molecule comprises or results from a 5′ breakpoint within chromosomal coordinates chr1: 206288099-206288139 and/or a 3′ breakpoint within chromosomal coordinates chr9: 133729488-133729528. In some embodiments, an ABL1 fusion nucleic acid molecule of the disclosure is a RALGPS1-ABL1 fusion nucleic acid molecule, wherein the RALGPS1-ABL1 fusion nucleic acid molecule comprises or results from a 5′ breakpoint within chromosomal coordinates chr9: 129694023-129694282 and/or a 3′ breakpoint within chromosomal coordinates chr9: 133730118-133730529. In some embodiments, an ABL1 fusion nucleic acid molecule of the disclosure is a TSC1-ABL1 fusion nucleic acid molecule, wherein the TSC1-ABL1 fusion nucleic acid molecule comprises or results from a 5′ breakpoint within chromosomal coordinates chr9:135,776,998-135,777,038 and/or a 3′ breakpoint within chromosomal coordinates chr9: 133729491-133729531. In some embodiments, an ABL1 fusion nucleic acid molecule of the disclosure is a TSPAN18-ABL1 fusion nucleic acid molecule, wherein the TSPAN18-ABL1 fusion nucleic acid molecule comprises or results from a 5′ breakpoint within chromosomal coordinates chr11: 44881929-44881969 and/or a 3′ breakpoint within chromosomal coordinates chr9: 133729517-133729557. In some embodiments, an ABL1 fusion nucleic acid molecule of the disclosure is a ZNF804B-ABL1 fusion nucleic acid molecule, wherein the ZNF804B-ABL1 fusion nucleic acid molecule comprises or results from a 5′ breakpoint within chromosomal coordinates chr7: 88521265-88521397 and/or a 3′ breakpoint within chromosomal coordinates chr9: 133729414-133729519. In some embodiments, an ABL1 fusion nucleic acid molecule of the disclosure is a BCR-ABL1 fusion nucleic acid molecule, wherein the BCR-ABL1 fusion nucleic acid molecule comprises or results from a 5′ breakpoint within chromosomal coordinates chr22: 23631687-23632492 and/or a 3′ breakpoint within chromosomal coordinates chr9: 133604425-133604577. In some embodiments, an ABL1 fusion nucleic acid molecule of the disclosure is a BCR-ABL1 fusion nucleic acid molecule, wherein the BCR-ABL1 fusion nucleic acid molecule comprises or results from a 5′ breakpoint within chromosomal coordinates chr22: 23632288-23632656 and/or a 3′ breakpoint within chromosomal coordinates chr9: 133663291-133663682. In some embodiments, an ABL1 fusion nucleic acid molecule of the disclosure is a BCR-ABL1 fusion nucleic acid molecule, wherein the BCR-ABL1 fusion nucleic acid molecule comprises or results from a 5′ breakpoint within chromosomal coordinates chr22: 23634171-23634647 and/or a 3′ breakpoint within chromosomal coordinates chr9: 133638886-133639512. In some embodiments, an ABL1 fusion nucleic acid molecule of the disclosure is a BCR-ABL1 fusion nucleic acid molecule, wherein the BCR-ABL1 fusion nucleic acid molecule comprises or results from a 5′ breakpoint within chromosomal coordinates chr22: 23633910-23634678 and/or a 3′ breakpoint within chromosomal coordinates chr9: 133724778-133725351. In some embodiments, an ABL1 fusion nucleic acid molecule of the disclosure is an ABL1-NUP214 fusion nucleic acid molecule, wherein the ABL1-NUP214 fusion nucleic acid molecule comprises or results from a 5′ breakpoint within chromosomal coordinates chr9: 134090583-134090623 and/or a 3′ breakpoint within chromosomal coordinates chr9: 133710830-133710870. In some embodiments, an ABL1 fusion nucleic acid molecule of the disclosure is an AIF1L-ABL1 fusion nucleic acid molecule, wherein the AIF1L-ABL1 fusion nucleic acid molecule comprises or results from a 5′ breakpoint within chromosomal coordinates chr9: 133978032-133978137 and/or a 3′ breakpoint within chromosomal coordinates chr9: 133729409-133729618. In some embodiments, an ABL1 fusion nucleic acid molecule of the disclosure is a EXOSC2-ABL1 fusion nucleic acid molecule, wherein the EXOSC2-ABL1 fusion nucleic acid molecule comprises or results from a 5′ breakpoint within chromosomal coordinates chr9: 133579154-133579531 and/or a 3′ breakpoint within chromosomal coordinates chr9: 133589786-133589985. In some embodiments, an ABL1 fusion nucleic acid molecule of the disclosure is a NUP214-ABL1 fusion nucleic acid molecule, wherein the NUP214-ABL1 fusion nucleic acid molecule comprises or results from a 5′ breakpoint within chromosomal coordinates chr9: 134106080-134106120 and/or a 3′ breakpoint within chromosomal coordinates chr9: 133730195-133730235. In some embodiments, an ABL1 fusion nucleic acid molecule of the disclosure is a NUP214-ABL1 fusion nucleic acid molecule, wherein the NUP214-ABL1 fusion nucleic acid molecule comprises or results from a 5′ breakpoint within chromosomal coordinates chr9: 134106030-134106070 and/or a 3′ breakpoint within chromosomal coordinates chr9: 133730218-133730258. In some embodiments, an ABL1 fusion nucleic acid molecule of the disclosure is a NUP214-ABL1 fusion nucleic acid molecule, wherein the NUP214-ABL1 fusion nucleic acid molecule comprises or results from a 5′ breakpoint within chromosomal coordinates chr9: 134106046-134106086 and/or a 3′ breakpoint within chromosomal coordinates chr9: 133730203-133730243. In some embodiments, an ABL1 fusion nucleic acid molecule of the disclosure is a NUP214-ABL1 fusion nucleic acid molecule, wherein the NUP214-ABL1 fusion nucleic acid molecule comprises or results from a 5′ breakpoint within chromosomal coordinates chr9: 134106028-134106068 and/or a 3′ breakpoint within chromosomal coordinates chr9: 133730234-133730274. In some embodiments, an ABL1 fusion nucleic acid molecule of the disclosure is a BCR-ABL1 fusion nucleic acid molecule, wherein the BCR-ABL1 fusion nucleic acid molecule comprises or results from a 5′ breakpoint within chromosomal coordinates chr22: 23524272-23524312 and/or a 3′ breakpoint within chromosomal coordinates chr9: 133730218-133730258. In some embodiments, an ABL1 fusion nucleic acid molecule of the disclosure is a SNX2-ABL1 fusion nucleic acid molecule, wherein the SNX2-ABL1 fusion nucleic acid molecule comprises or results from a 5′ breakpoint within chromosomal coordinates chr5: 122135451-122135491 and/or a 3′ breakpoint within chromosomal coordinates chr9: 133738222-133738262. In some embodiments, an ABL1 fusion nucleic acid molecule of the disclosure is a SNX2-ABL1 fusion nucleic acid molecule, wherein the SNX2-ABL1 fusion nucleic acid molecule comprises or results from a 5′ breakpoint within chromosomal coordinates chr5: 122135445-122135485 and/or a 3′ breakpoint within chromosomal coordinates chr9: 133738214-133738254. In some embodiments, an ABL1 fusion nucleic acid molecule of the disclosure is a BCR-ABL1 fusion nucleic acid molecule, wherein the BCR-ABL1 fusion nucleic acid molecule comprises or results from a 5′ breakpoint within chromosomal coordinates chr22: 23631693-23632188 and/or a 3′ breakpoint within chromosomal coordinates chr9: 133607683-133608245. In some embodiments, an ABL1 fusion nucleic acid molecule of the disclosure is a BMP2K-ABL1 fusion nucleic acid molecule, wherein the BMP2K-ABL1 fusion nucleic acid molecule comprises or results from a 5′ breakpoint within chromosomal coordinates chr4: 79800022-79800062 and/or a 3′ breakpoint within chromosomal coordinates chr9: 133730205-133730245.

In some embodiments, an ABL1 fusion nucleic acid molecule of the disclosure is an ABL1 fusion nucleic acid molecule as listed in Table 2 in Example 1 herein, comprising or resulting from the corresponding exonic/intronic and/or chromosomal breakpoints as provided in Table 2 in Example 1 herein.

### Exons in Exemplary ABL1 Fusions

In some embodiments, an ABL1 fusion nucleic acid molecule of the disclosure is an EHMT1-ABL1 fusion nucleic acid molecule, comprising a fusion between exon 4, or a portion thereof, of an EHMT1 gene and exon 2, or a portion thereof, of an ABL1 gene. In some embodiments, the EHMT1-ABL1 fusion nucleic acid molecule comprises, in 5′ to 3′ direction, at least a portion of an EHMT1 gene fused to at least a portion of an ABL1 gene. In some embodiments, an ABL1 fusion nucleic acid molecule of the disclosure is a MED27-ABL1 fusion nucleic acid molecule, comprising a fusion between exon 4, or a portion thereof, of a MED27 gene and exon 5, or a portion thereof, of an ABL1 gene. In some embodiments, the MED27-ABL1 fusion nucleic acid molecule comprises, in the 5′ to 3′ direction, at least a portion of a MED27 gene fused to at least a portion of an ABL1 gene. In some embodiments, an ABL1 fusion nucleic acid molecule of the disclosure is an RFFL-ABL1 fusion nucleic acid molecule, comprising a fusion between exon 5, or a portion thereof, of an RFFL gene and exon 2, or a portion thereof, of an ABL1 gene. In some embodiments, the RFFL-ABL1 fusion nucleic acid molecule comprises, in 5′ to 3′ direction, at least a portion of an RFFL gene fused to at least a portion of an ABL1 gene. In some embodiments, an ABL1 fusion nucleic acid molecule of the disclosure is an SLC27A4-ABL1 fusion nucleic acid molecule, comprising a fusion between exon 12, or a portion thereof, of an SLC27A4 gene and exon 6, or a portion thereof, of an ABL1 gene. In some embodiments, the SLC27A4-ABL1 fusion nucleic acid molecule comprises, in 5′ to 3′ direction, at least a portion of an SLC27A4 gene fused to at least a portion of an ABL1 gene. In some embodiments, an ABL1 fusion nucleic acid molecule of the disclosure is a BCR-ABL1 fusion nucleic acid molecule, comprising a fusion between exon 13, or a portion thereof, of a BCR gene and exon 2, or a portion thereof, of an ABL1 gene. In some embodiments, the BCR-ABL1 fusion nucleic acid molecule comprises, in 5′ to 3′ direction, at least a portion of a BCR gene fused to at least a portion of an ABL1 gene. In some embodiments, an ABL1 fusion nucleic acid molecule of the disclosure is a BCR-ABL1 fusion nucleic acid molecule, comprising a fusion between exon 14, or a portion thereof, of a BCR gene and exon 2, or a portion thereof, of an ABL1 gene. In some embodiments, the BCR-ABL1 fusion comprises, in 5′ to 3′ direction, at least a portion of a BCR gene fused to at least a portion of an ABL1 gene. In some embodiments, an ABL1 fusion nucleic acid molecule of the disclosure is a BCR-ABL1 fusion nucleic acid molecule, comprising a fusion between exon 1, or a portion thereof, of a BCR gene and exon 3, or a portion thereof, of an ABL1 gene. In some embodiments, the BCR-ABL1 fusion nucleic acid molecule comprises, in 5′ to 3′ direction, at least a portion of a BCR gene fused to at least a portion of an ABL1 gene. In some embodiments, an ABL1 fusion nucleic acid molecule of the disclosure is a BCR-ABL1 fusion nucleic acid molecule, comprising a fusion between exon 12, or a portion thereof, of a BCR gene and exon 2, or a portion thereof, of an ABL1 gene. In some embodiments, the BCR-ABL1 fusion nucleic acid molecule comprises, in 5′ to 3′ direction, at least a portion of a BCR gene fused to at least a portion of an ABL1 gene. In some embodiments, an ABL1 fusion nucleic acid molecule of the disclosure is a NUP214-ABL1 fusion nucleic acid molecule, comprising a fusion between exon 34, or a portion thereof, of a NUP214 gene and exon 3, or a portion thereof, of an ABL1 gene. In some embodiments, the NUP214-ABL1 fusion nucleic acid molecule comprises, in 5′ to 3′ direction, at least a portion of a NUP214 gene fused to at least a portion of an ABL1 gene. In some embodiments, an ABL1 fusion nucleic acid molecule of the disclosure is an ABL1-AIF1L fusion nucleic acid molecule, comprising a fusion between exon 10, or a portion thereof, of an ABL1 gene and exon 4, or a portion thereof, of an AIF1L gene. In some embodiments, the ABL1-AIF1L fusion nucleic acid molecule comprises, in 5′ to 3′ direction, at least a portion of an ABL1 gene fused to at least a portion of an AIF1L gene. In some embodiments, an ABL1 fusion nucleic acid molecule of the disclosure is an RCSD1-ABL1 fusion nucleic acid molecule, comprising a fusion between exon 4, or a portion thereof, of an RCSD1 gene and exon 4, or a portion thereof, of an ABL1 gene. In some embodiments, the RCSD1-ABL1 fusion nucleic acid molecule comprises, in 5′ to 3′ direction, at least a portion of an RCSD1 gene fused to at least a portion of an ABL1 gene. In some embodiments, an ABL1 fusion nucleic acid molecule of the disclosure is an APOOL-ABL1 fusion nucleic acid molecule, comprising a fusion between exon 2, or a portion thereof, of an APOOL gene and exon 2, or a portion thereof, of an ABL1 gene. In some embodiments, the APOOL-ABL1 fusion nucleic acid molecule comprises, in 5′ to 3′ direction, at least a portion of an APOOL gene fused to at least a portion of an ABL1 gene. In some embodiments, an ABL1 fusion nucleic acid molecule of the disclosure is an ABL1-DNAH2 fusion nucleic acid molecule, comprising a fusion between exon 10, or a portion thereof, of an ABL1 gene and exon 4, or a portion thereof, of a DNAH2 gene. In some embodiments, the ABL1-DNAH2 fusion nucleic acid molecule comprises, in 5′ to 3′ direction, at least a portion of an ABL1 gene fused to at least a portion of a DNAH2 gene. In some embodiments, an ABL1 fusion nucleic acid molecule of the disclosure is a RHEX/C1orf186-ABL1 fusion nucleic acid molecule, comprising a fusion between exon 1, or a portion thereof, of a RHEX/C1orf186 gene and exon 2, or a portion thereof, of an ABL1 gene. In some embodiments, the RHEX/C1orf186-ABL1 fusion nucleic acid molecule comprises, in 5′ to 3′ direction, at least a portion of a RHEX/C1orf186-gene fused to at least a portion of an ABL1 gene. In some embodiments, an ABL1 fusion nucleic acid molecule of the disclosure is a RALGPS1-ABL1 fusion nucleic acid molecule, comprising a fusion between exon 1, or a portion thereof, of a RALGPS1 gene and exon 3, or a portion thereof, of an ABL1 gene. In some embodiments, the RALGPS1-ABL1 fusion nucleic acid molecule comprises, in 5′ to 3′ direction, at least a portion of a RALGPS1 gene fused to at least a portion of an ABL1 gene. In some embodiments, an ABL1 fusion nucleic acid molecule of the disclosure is a TSC1-ABL1 fusion nucleic acid molecule, comprising a fusion between exon 19, or a portion thereof, of a TSC1 gene and exon 2, or a portion thereof, of an ABL1 gene. In some embodiments, the TSC1-ABL1 fusion nucleic acid molecule comprises, in 5′ to 3′ direction, at least a portion of a TSC1 gene fused to at least a portion of an ABL1 gene. In some embodiments, an ABL1 fusion nucleic acid molecule of the disclosure is a TSPAN18-ABL1 fusion nucleic acid molecule, comprising a fusion between exon 2, or a portion thereof, of a TSPAN18 gene and exon 2, or a portion thereof, of an ABL1 gene. In some embodiments, the TSPAN18-ABL1 fusion nucleic acid molecule comprises, in 5′ to 3′ direction, at least a portion of a TSPAN18 gene fused to at least a portion of an ABL1 gene. In some embodiments, an ABL1 fusion nucleic acid molecule of the disclosure is a ZNF804B-ABL1 fusion nucleic acid molecule, comprising a fusion between exon 1, or a portion thereof, of a ZNF804B gene and exon 2, or a portion thereof, of an ABL1 gene. In some embodiments, the ZNF804B-ABL1 fusion comprises, in 5′ to 3′ direction, at least a portion of a ZNF804B gene fused to at least a portion of an ABL1 gene. In some embodiments, an ABL1 fusion nucleic acid molecule of the disclosure is an ABL1-NUP214 fusion nucleic acid molecule, comprising a fusion between exon 1, or a portion thereof, of an ABL1 gene and exon 31, or a portion thereof, of a NUP214 gene. In some embodiments, the ABL1-NUP214 fusion nucleic acid molecule comprises, in the 5′ to 3′ direction, at least a portion of an ABL1 gene fused to at least a portion of a NUP214 gene. In some embodiments, an ABL1 fusion nucleic acid molecule of the disclosure is an AIF1L-ABL1 fusion nucleic acid molecule, comprising a fusion between exon 2, or a portion thereof, of an AIF1L gene and exon 2, or a portion thereof, of an ABL1 gene. In some embodiments, the AIF1L-ABL1 fusion comprises, in 5′ to 3′ direction, at least a portion of an AIF1L gene fused to at least a portion of an ABL1 gene. In some embodiments, an ABL1 fusion nucleic acid molecule of the disclosure is an EXOSC2-ABL1 fusion nucleic acid molecule, comprising a fusion between exon 9, or a portion thereof, of an EXOSC2 gene and exon 1, or a portion thereof, of an ABL1 gene. In some embodiments, the EXOSC2-ABL1 fusion nucleic acid molecule comprises, in 5′ to 3′ direction, at least a portion of an EXOSC2 gene fused to at least a portion of an ABL1 gene. In some embodiments, an ABL1 fusion nucleic acid molecule of the disclosure is an SNX2-ABL1 fusion nucleic acid molecule, comprising a fusion between exon 3, or a portion thereof, of an SNX2 gene and exon 4, or a portion thereof, of an ABL1 gene. In some embodiments, the SNX2-ABL1 fusion nucleic acid molecule comprises, in 5′ to 3′ direction, at least a portion of an SNX2 gene fused to at least a portion of an ABL1 gene. In some embodiments, an ABL1 fusion nucleic acid molecule of the disclosure is a BMP2K-ABL1 fusion nucleic acid molecule, comprising a fusion between exon 15, or a portion thereof, of a BMP2K gene and exon 3, or a portion thereof, of an ABL1 gene. In some embodiments, the BMP2K-ABL1 fusion comprises, in 5′ to 3′ direction, at least a portion of a BMP2K gene fused to at least a portion of an ABL1 gene.

In some embodiments, an ABL1 fusion nucleic acid molecule of the disclosure is an EHMT1-ABL1 fusion nucleic acid molecule comprising, in 5′ to 3′ direction, exons 1-3 and exon 4, or a portion thereof, of an EHMT1 gene, fused to exon 2, or a portion thereof, and exons 3-11 of an ABL1 gene. In some embodiments, an ABL1 fusion nucleic acid molecule of the disclosure is a MED27-ABL1 fusion nucleic acid molecule comprising, in 5′ to 3′ direction, exons 1-3 and exon 4, or a portion thereof, of a MED27 gene, fused to exon 5, or a portion thereof, and exons 6-11 of an ABL1 gene. In some embodiments, an ABL1 fusion nucleic acid molecule of the disclosure is an RFFL-ABL1 fusion nucleic acid molecule comprising, in 5′ to 3′ direction, exons 1-4 and exon 5, or a portion thereof, of an RFFL gene, fused to exon 2, or a portion thereof, and exons 3-11 of an ABL1 gene. In some embodiments, an ABL1 fusion nucleic acid molecule of the disclosure is a SLC27A4-ABL1 fusion nucleic acid molecule comprising, in 5′ to 3′ direction, exons 1-11 and exon 12, or a portion thereof, of an SLC27A4 gene, fused to exon 6, or a portion thereof, and exons 7-11 of an ABL1 gene. In some embodiments, an ABL1 fusion nucleic acid molecule of the disclosure is a BCR-ABL1 fusion nucleic acid molecule comprising, in 5′ to 3′ direction, exons 1-12 and exon 13, or a portion thereof, of a BCR gene, fused to exon 2, or a portion thereof, and exons 3-11 of an ABL1 gene. In some embodiments, an ABL1 fusion nucleic acid molecule of the disclosure is a BCR-ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene. In some embodiments, an ABL1 fusion nucleic acid molecule of the disclosure is a NUP214-ABL1 fusion nucleic acid molecule comprising, in 5′ to 3′ direction, exons 1-33 and exon 34, or a portion thereof, of a NUP214 gene, fused to exon 3, or a portion thereof, and exons 4-11 of an ABL1 gene. In some embodiments, an ABL1 fusion nucleic acid molecule of the disclosure is an ABL1-AIF1L fusion nucleic acid molecule comprising, in 5′ to 3′ direction, exons 1-9 and exon 10, or a portion thereof, of an ABL1 gene, fused to exon 4, or a portion thereof, and exons 5-6 of an AIF1L gene. In some embodiments, an ABL1 fusion nucleic acid molecule of the disclosure is an RCSD1-ABL1 fusion nucleic acid molecule comprising, in 5′ to 3′ direction, exons 1-3 and exon 4, or a portion thereof, of an RCSD1 gene, fused to exon 4, or a portion thereof, and exons 5-11 of an ABL1 gene. In some embodiments, an ABL1 fusion nucleic acid molecule of the disclosure is an APOOL-ABL1 fusion nucleic acid molecule comprising, in 5′ to 3′ direction, exon 1 and exon 2, or a portion thereof, of an APOOL gene, fused to exon 2, or a portion thereof, and exons 3-11 of an ABL1 gene. In some embodiments, an ABL1 fusion nucleic acid molecule of the disclosure is an ABL1-DNAH2 fusion nucleic acid molecule comprising, in 5′ to 3′ direction, exons 1-9 and exon 10, or a portion thereof, of an ABL1 gene, fused to exon 4, or a portion thereof, and exons 5-85 of a DNAH2 gene. In some embodiments, an ABL1 fusion nucleic acid molecule of the disclosure is a RHEX/C1orf186-ABL1 fusion nucleic acid molecule comprising, in 5′ to 3′ direction, exon 1, or a portion thereof, of a RHEX/C1orf186 gene, fused to exon 2, or a portion thereof, and exons 3-11 of an ABL1 gene. In some embodiments, an ABL1 fusion nucleic acid molecule of the disclosure is a RALGPS1-ABL1 fusion nucleic acid molecule comprising, in 5′ to 3′ direction, exon 1, or a portion thereof, of a RALGPS1 gene, fused to exon 3, or a portion thereof, and exons 4-11 of an ABL1 gene. In some embodiments, an ABL1 fusion nucleic acid molecule of the disclosure is a TSC1-ABL1 fusion nucleic acid molecule comprising, in 5′ to 3′ direction, exons 1-18 and exon 19, or a portion thereof, of a TSC1 gene, fused to exon 2, or a portion thereof, and exons 3-11 of an ABL1 gene. In some embodiments, an ABL1 fusion nucleic acid molecule of the disclosure is a TSPAN18-ABL1 fusion nucleic acid molecule comprising, in 5′ to 3′ direction, exon 1 and exon 2, or a portion thereof, of a TSPAN18 gene, fused to exon 2, or a portion thereof, and exons 3-11 of an ABL1 gene. In some embodiments, an ABL1 fusion nucleic acid molecule of the disclosure is a ZNF804B-ABL1 fusion nucleic acid molecule comprising, in 5′ to 3′ direction, exon 1, or a portion thereof, of a ZNF804B gene, fused to exon 2, or a portion thereof, and exons 3-11 of an ABL1 gene. In some embodiments, an ABL1 fusion nucleic acid molecule of the disclosure is an ABL1-NUP214 fusion nucleic acid molecule comprising, in 5′ to 3′ direction, exon 1, or a portion thereof, of an ABL1 gene, fused to exon 31, or a portion thereof, and exons 32-36 of a NUP214 gene. In some embodiments, an ABL1 fusion nucleic acid molecule of the disclosure is an AIF1L-ABL1 fusion nucleic acid molecule comprising, in 5′ to 3′ direction, exon 1 and exon 2, or a portion thereof, of an AIF1L gene, fused to exon 2, or a portion thereof, and exons 3-11 of an ABL1 gene. In some embodiments, an ABL1 fusion nucleic acid molecule of the disclosure is an EXOSC2-ABL1 fusion nucleic acid molecule comprising, in 5′ to 3′ direction, exons 1-8 and exon 9, or a portion thereof, of an EXOSC2 gene, fused to exon 1, or a portion thereof, and exons 2-11 of an ABL1 gene. In some embodiments, an ABL1 fusion nucleic acid molecule of the disclosure is a BCR-ABL1 fusion nucleic acid molecule comprising, in 5′ to 3′ direction, exon 1, or a portion thereof, of a BCR gene, fused to exon 3, or a portion thereof, and exons 4-11 of an ABL1 gene. In some embodiments, an ABL1 fusion nucleic acid molecule of the disclosure is an SNX2-ABL1 fusion nucleic acid molecule comprising, in 5′ to 3′ direction, exons 1-2 and exon 3, or a portion thereof, of an SNX2 gene, fused to exon 4, or a portion thereof, and exons 5-11 of an ABL1 gene. In some embodiments, an ABL1 fusion nucleic acid molecule of the disclosure is a BCR-ABL1 fusion nucleic acid molecule comprising, in 5′ to 3′ direction, exons 1-11 and exon 12, or a portion thereof, of a BCR gene, fused to exon 2, or a portion thereof, and exons 3-11 of an ABL1 gene. In some embodiments, an ABL1 fusion nucleic acid molecule of the disclosure is a BMP2K-ABL1 fusion nucleic acid molecule comprising, in 5′ to 3′ direction, exons 1-14 and exon 15, or a portion thereof, of a BMP2K gene, fused to exon 3, or a portion thereof, and exons 4-11 of an ABL1 gene.

In some embodiments, an ABL1 fusion nucleic acid molecule of the disclosure is an ABL1 fusion nucleic acid molecule as listed in Table 2 in Example 1 herein, e.g., comprising the corresponding exons (or portions thereof) as listed in Table 2 in Example 1 herein.

### Sequences of Exemplary ABL1 Fusions and Encoded ABL1 Fusion Polypeptides

In some embodiments, an ABL1 fusion nucleic acid molecule of the disclosure is an EHMT1-ABL1 fusion nucleic acid molecule, wherein the EHMT1-ABL1 fusion nucleic acid molecule encodes an EHMT1-ABL1 fusion polypeptide comprising the amino acid sequence of SEQ ID NO: 23, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 23. In some embodiments, the EHMT1-ABL1 fusion nucleic acid molecule comprises the nucleotide sequence of SEQ ID NO: 2, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 2.

(SEQ ID NO: 2)
ATGGCCGCCGCCGATGCCGAGGCAGTTCCGGCGAGGGGGGAGCCTCAGCAGGATTGCTGTGTGAAAAC
CGAGCTGCTGGGAGAAGAGACACCTATGGCTGCCGATGAAGGCTCAGCAGAGAAACAGGCAGGAGAGG
CCCACATGGCTGCGGACGGTGAGACCAATGGGTCTTGTGAAAACAGCGATGCCAGCAGTCATGCAAAT
GCTGCAAAGCACACTCAGGACAGCGCAAGGGTCAACCCCCAGGATGGCACCAACACACTAACTCGGAT
AGCGGAAAATGGGGTTTCAGAAAGAGACTCAGAAGCGGCGAAGCAAAACCACGTCACTGCCGACGACT
TTGTGCAGACTTCTGTCATCGGCAGCAACGGATACATCTTAAATAAGCCGGCCCTACAGGCACAGCCC
TTGAGGACTACCAGCACTCTGGCCTCTTCGCTGCCTGGCCATGCTGCAAAAACCCTTCCTGGAGGGGC
TGGCAAAGGCAGGACTCCAAGCGCTTTTCCCCAGACGCCAGCCGCCCCACCAGCCACCCTTGGGGAGG
GGAGTGCTGACACAGAGGACAGGAAGCTCCCGGCCCCTGGCGCCGACGTCAAGGTCCACAGGGCACGC
AAGACCATGCCGAAGTCCGTCGTGGGCCTGCATGCAGCCAGTAAAGATCCCAGAGAAGTTCGAGAAGC
TAGAGATCATAAGGAACCAAAAGAGGAGATCAACAAAAACATTTCTGACTTTGGACGACAGCAGCTTT
TACCCCCCTTCCCATCCCTTCATCAGTCGCTACCTCAGAACCAGTGCTACATGGCCACCACAAAATCA
CAGACAGAAGCCCTTCAGCGGCCAGTAGCATCTGACTTTGAGCCTCAGGGTCTGAGTGAAGCCGCTCG
TTGGAACTCCAAGGAAAACCTTCTCGCTGGACCCAGTGAAAATGACCCCAACCTTTTCGTTGCACTGT
ATGATTTTGTGGCCAGTGGAGATAACACTCTAAGCATAACTAAAGGTGAAAAGCTCCGGGTCTTAGGC
TATAATCACAATGGGGAATGGTGTGAAGCCCAAACCAAAAATGGCCAAGGCTGGGTCCCAAGCAACTA
CATCACGCCAGTCAACAGTCTGGAGAAACACTCCTGGTACCATGGGCCTGTGTCCCGCAATGCCGCTG
AGTATCTGCTGAGCAGCGGGATCAATGGCAGCTTCTTGGTGCGTGAGAGTGAGAGCAGTCCTGGCCAG
AGGTCCATCTCGCTGAGATACGAAGGGAGGGTGTACCATTACAGGATCAACACTGCTTCTGATGGCAA
GCTCTACGTCTCCTCCGAGAGCCGCTTCAACACCCTGGCCGAGTTGGTTCATCATCATTCAACGGTGG
CCGACGGGCTCATCACCACGCTCCATTATCCAGCCCCAAAGCGCAACAAGCCCACTGTCTATGGTGTG
TCCCCCAACTACGACAAGTGGGAGATGGAACGCACGGACATCACCATGAAGCACAAGCTGGGCGGGGG
CCAGTACGGGGAGGTGTACGAGGGCGTGTGGAAGAAATACAGCCTGACGGTGGCCGTGAAGACCTTGA
AGGAGGACACCATGGAGGTGGAAGAGTTCTTGAAAGAAGCTGCAGTCATGAAAGAGATCAAACACCCT
AACCTGGTGCAGCTCCTTGGGGTCTGCACCCGGGAGCCCCCGTTCTATATCATCACTGAGTTCATGAC
CTACGGGAACCTCCTGGACTACCTGAGGGAGTGCAACCGGCAGGAGGTGAACGCCGTGGTGCTGCTGT
ACATGGCCACTCAGATCTCGTCAGCCATGGAGTACCTGGAGAAGAAAAACTTCATCCACAGAGATCTT
GCTGCCCGAAACTGCCTGGTAGGGGAGAACCACTTGGTGAAGGTAGCTGATTTTGGCCTGAGCAGGTT
GATGACAGGGGACACCTACACAGCCCATGCTGGAGCCAAGTTCCCCATCAAATGGACTGCACCCGAGA
GCCTGGCCTACAACAAGTTCTCCATCAAGTCCGACGTCTGGGCATTTGGAGTATTGCTTTGGGAAATT
GCTACCTATGGCATGTCCCCTTACCCGGGAATTGACCTGTCCCAGGTGTATGAGCTGCTAGAGAAGGA
CTACCGCATGGAGCGCCCAGAAGGCTGCCCAGAGAAGGTCTATGAACTCATGCGAGCATGTTGGCAGT
GGAATCCCTCTGACCGGCCCTCCTTTGCTGAAATCCACCAAGCCTTTGAAACAATGTTCCAGGAATCC
AGTATCTCAGACGAAGTGGAAAAGGAGCTGGGGAAACAAGGCGTCCGTGGGGCTGTGAGTACCTTGCT
GCAGGCCCCAGAGCTGCCCACCAAGACGAGGACCTCCAGGAGAGCTGCAGAGCACAGAGACACCACTG
ACGTGCCTGAGATGCCTCACTCCAAGGGCCAGGGAGAGAGCGATCCTCTGGACCATGAGCCTGCCGTG
TCTCCATTGCTCCCTCGAAAAGAGCGAGGTCCCCCGGAGGGCGGCCTGAATGAAGATGAGCGCCTTCT
CCCCAAAGACAAAAAGACCAACTTGTTCAGCGCCTTGATCAAGAAGAAGAAGAAGACAGCCCCAACCC
CTCCCAAACGCAGCAGCTCCTTCCGGGAGATGGACGGCCAGCCGGAGCGCAGAGGGGCCGGCGAGGAA
GAGGGCCGAGACATCAGCAACGGGGCACTGGCTTTCACCCCCTTGGACACAGCTGACCCAGCCAAGTC
CCCAAAGCCCAGCAATGGGGCTGGGGTCCCCAATGGAGCCCTCCGGGAGTCCGGGGGCTCAGGCTTCC
GGTCTCCCCACCTGTGGAAGAAGTCCAGCACGCTGACCAGCAGCCGCCTAGCCACCGGCGAGGAGGAG
GGCGGTGGCAGCTCCAGCAAGCGCTTCCTGCGCTCTTGCTCCGCCTCCTGCGTTCCCCATGGGGCCAA
GGACACGGAGTGGAGGTCAGTCACGCTGCCTCGGGACTTGCAGTCCACGGGAAGACAGTTTGACTCGT
CCACATTTGGAGGGCACAAAAGTGAGAAGCCGGCTCTGCCTCGGAAGAGGGCAGGGGAGAACAGGTCT
GACCAGGTGACCCGAGGCACAGTAACGCCTCCCCCCAGGCTGGTGAAAAAGAATGAGGAAGCTGCTGA
TGAGGTCTTCAAAGACATCATGGAGTCCAGCCCGGGCTCCAGCCCGCCCAACCTGACTCCAAAACCCC
TCCGGCGGCAGGTCACCGTGGCCCCTGCCTCGGGCCTCCCCCACAAGGAAGAAGCTGGAAAGGGCAGT
GCCTTAGGGACCCCTGCTGCAGCTGAGCCAGTGACCCCCACCAGCAAAGCAGGCTCAGGTGCACCAGG
GGGCACCAGCAAGGGCCCCGCCGAGGAGTCCAGAGTGAGGAGGCACAAGCACTCCTCTGAGTCGCCAG
GGAGGGACAAGGGGAAATTGTCCAGGCTCAAACCTGCCCCGCCGCCCCCACCAGCAGCCTCTGCAGGG
AAGGCTGGAGGAAAGCCCTCGCAGAGCCCGAGCCAGGAGGCGGCCGGGGAGGCAGTCCTGGGCGCAAA
GACAAAAGCCACGAGTCTGGTTGATGCTGTGAACAGTGACGCTGCCAAGCCCAGCCAGCCGGGAGAGG
GCCTCAAAAAGCCCGTGCTCCCGGCCACTCCAAAGCCACAGTCCGCCAAGCCGTCGGGGACCCCCATC
AGCCCAGCCCCCGTTCCCTCCACGTTGCCATCAGCATCCTCGGCCCTGGCAGGGGACCAGCCGTCTTC
CACCGCCTTCATCCCTCTCATATCAACCCGAGTGTCTCTTCGGAAAACCCGCCAGCCTCCAGAGCGGA
TCGCCAGCGGCGCCATCACCAAGGGCGTGGTCCTGGACAGCACCGAGGCGCTGTGCCTCGCCATCTCT
AGGAACTCCGAGCAGATGGCCAGCCACAGCGCAGTGCTGGAGGCCGGCAAAAACCTCTACACGTTCTG
CGTGAGCTATGTGGATTCCATCCAGCAAATGAGGAACAAGTTTGCCTTCCGAGAGGCCATCAACAAAC
TGGAGAATAATCTCCGGGAGCTTCAGATCTGCCCGGCGACAGCAGGCAGTGGTCCAGCGGCCACTCAG
GACTTCAGCAAGCTCCTCAGTTCGGTGAAGGAAATCAGTGACATAGTGCAGAGGTAG

In some embodiments, an ABL1 fusion nucleic acid molecule of the disclosure is a MED27-ABL1 fusion nucleic acid molecule, wherein the MED27-ABL1 fusion nucleic acid molecule encodes a MED27-ABL1 fusion polypeptide comprising the amino acid sequence of SEQ ID NO: 24, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 24. In some embodiments, the MED27-ABL1 fusion nucleic acid molecule comprises the nucleotide sequence of SEQ ID NO: 3, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 3.

(SEQ ID NO: 3)
ATGGCGGACGTGATAAATGTCAGTGTGAACCTGGAGGCCTTTTCCCAGGCCATTAGTGCCATCCAGGC
GCTGCGCTCCAGCGTGAGCAGGGTGTTCGACTGCCTGAAGGATGGGATGCGGAACAAGGAGACGCTGG
AGGGCCGGGAGAAGGCCTTTATTGCGCACTTCCAGGACAACTTACATTCGGTCAACCGGGACCTCAAT
GAGCTGGAACGTCTGAGCAATCTGGTAGGCAAGCCATCTGAGAACCATCCTCTTCATAACAGTGGGCT
GTTAAGCCTGGATCCTGTGCAGGACAAAACTCCTCTCTATAGTCAACTCCTTCAAGCATATAAGTGGT
CAAACAAGTTGCAGTACCATGCAGGACTAGCATCTGGCCTTTTAAATCAGCAGTCATTGAAGCGTTCC
GCTAATCAGATGGGAGTATCTGCCAAACGTAGACCAAAGGCTCAGCCCACAACTCTTGTCCTACCACC
TCAATATGTTGATGATGTGATCAGCCGCATTGACAGGATGTTTCCTGAAATGTCCATCCACTTATCCA
GACCCAATGGAACATCAGCAATGCTTCTGGAGGACACCATGGAGGTGGAAGAGTTCTTGAAAGAAGCT
GCAGTCATGAAAGAGATCAAACACCCTAACCTGGTGCAGCTCCTTGGGGTCTGCACCCGGGAGCCCCC
GTTCTATATCATCACTGAGTTCATGACCTACGGGAACCTCCTGGACTACCTGAGGGAGTGCAACCGGC
AGGAGGTGAACGCCGTGGTGCTGCTGTACATGGCCACTCAGATCTCGTCAGCCATGGAGTACCTGGAG
AAGAAAAACTTCATCCACAGAGATCTTGCTGCCCGAAACTGCCTGGTAGGGGAGAACCACTTGGTGAA
GGTAGCTGATTTTGGCCTGAGCAGGTTGATGACAGGGGACACCTACACAGCCCATGCTGGAGCCAAGT
TCCCCATCAAATGGACTGCACCCGAGAGCCTGGCCTACAACAAGTTCTCCATCAAGTCCGACGTCTGG
GCATTTGGAGTATTGCTTTGGGAAATTGCTACCTATGGCATGTCCCCTTACCCGGGAATTGACCTGTC
CCAGGTGTATGAGCTGCTAGAGAAGGACTACCGCATGGAGCGCCCAGAAGGCTGCCCAGAGAAGGTCT
ATGAACTCATGCGAGCATGTTGGCAGTGGAATCCCTCTGACCGGCCCTCCTTTGCTGAAATCCACCAA
GCCTTTGAAACAATGTTCCAGGAATCCAGTATCTCAGACGAAGTGGAAAAGGAGCTGGGGAAACAAGG
CGTCCGTGGGGCTGTGAGTACCTTGCTGCAGGCCCCAGAGCTGCCCACCAAGACGAGGACCTCCAGGA
GAGCTGCAGAGCACAGAGACACCACTGACGTGCCTGAGATGCCTCACTCCAAGGGCCAGGGAGAGAGC
GATCCTCTGGACCATGAGCCTGCCGTGTCTCCATTGCTCCCTCGAAAAGAGCGAGGTCCCCCGGAGGG
CGGCCTGAATGAAGATGAGCGCCTTCTCCCCAAAGACAAAAAGACCAACTTGTTCAGCGCCTTGATCA
AGAAGAAGAAGAAGACAGCCCCAACCCCTCCCAAACGCAGCAGCTCCTTCCGGGAGATGGACGGCCAG
CCGGAGCGCAGAGGGGCCGGCGAGGAAGAGGGCCGAGACATCAGCAACGGGGCACTGGCTTTCACCCC
CTTGGACACAGCTGACCCAGCCAAGTCCCCAAAGCCCAGCAATGGGGCTGGGGTCCCCAATGGAGCCC
TCCGGGAGTCCGGGGGCTCAGGCTTCCGGTCTCCCCACCTGTGGAAGAAGTCCAGCACGCTGACCAGC
AGCCGCCTAGCCACCGGCGAGGAGGAGGGCGGTGGCAGCTCCAGCAAGCGCTTCCTGCGCTCTTGCTC
CGCCTCCTGCGTTCCCCATGGGGCCAAGGACACGGAGTGGAGGTCAGTCACGCTGCCTCGGGACTTGC
AGTCCACGGGAAGACAGTTTGACTCGTCCACATTTGGAGGGCACAAAAGTGAGAAGCCGGCTCTGCCT
CGGAAGAGGGCAGGGGAGAACAGGTCTGACCAGGTGACCCGAGGCACAGTAACGCCTCCCCCCAGGCT
GGTGAAAAAGAATGAGGAAGCTGCTGATGAGGTCTTCAAAGACATCATGGAGTCCAGCCCGGGCTCCA
GCCCGCCCAACCTGACTCCAAAACCCCTCCGGCGGCAGGTCACCGTGGCCCCTGCCTCGGGCCTCCCC
CACAAGGAAGAAGCTGGAAAGGGCAGTGCCTTAGGGACCCCTGCTGCAGCTGAGCCAGTGACCCCCAC
CAGCAAAGCAGGCTCAGGTGCACCAGGGGGCACCAGCAAGGGCCCCGCCGAGGAGTCCAGAGTGAGGA
GGCACAAGCACTCCTCTGAGTCGCCAGGGAGGGACAAGGGGAAATTGTCCAGGCTCAAACCTGCCCCG
CCGCCCCCACCAGCAGCCTCTGCAGGGAAGGCTGGAGGAAAGCCCTCGCAGAGCCCGAGCCAGGAGGC
GGCCGGGGAGGCAGTCCTGGGCGCAAAGACAAAAGCCACGAGTCTGGTTGATGCTGTGAACAGTGACG
CTGCCAAGCCCAGCCAGCCGGGAGAGGGCCTCAAAAAGCCCGTGCTCCCGGCCACTCCAAAGCCACAG
TCCGCCAAGCCGTCGGGGACCCCCATCAGCCCAGCCCCCGTTCCCTCCACGTTGCCATCAGCATCCTC
GGCCCTGGCAGGGGACCAGCCGTCTTCCACCGCCTTCATCCCTCTCATATCAACCCGAGTGTCTCTTC
GGAAAACCCGCCAGCCTCCAGAGCGGATCGCCAGCGGCGCCATCACCAAGGGCGTGGTCCTGGACAGC
ACCGAGGCGCTGTGCCTCGCCATCTCTAGGAACTCCGAGCAGATGGCCAGCCACAGCGCAGTGCTGGA
GGCCGGCAAAAACCTCTACACGTTCTGCGTGAGCTATGTGGATTCCATCCAGCAAATGAGGAACAAGT
TTGCCTTCCGAGAGGCCATCAACAAACTGGAGAATAATCTCCGGGAGCTTCAGATCTGCCCGGCGACA
GCAGGCAGTGGTCCAGCGGCCACTCAGGACTTCAGCAAGCTCCTCAGTTCGGTGAAGGAAATCAGTGA
CATAGTGCAGAGGTAG

In some embodiments, an ABL1 fusion nucleic acid molecule of the disclosure is an RFFL-ABL1 fusion nucleic acid molecule, wherein the RFFL-ABL1 fusion nucleic acid molecule encodes a RFFL-ABL1 fusion polypeptide comprising the amino acid sequence of SEQ ID NO: 25, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 25. In some embodiments, the RFFL-ABL1 fusion nucleic acid molecule comprises the nucleotide sequence of SEQ ID NO: 4, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 4.

(SEQ ID NO: 4)
ATGTGGGCAACCTGCTGCAACTGGTTCTGCCTGGATGGACAGCCTGAGGAGGTCCCACCACCCCAGGG
AGCCAGGATGCAGGCCTATTCCAACCCTGGGTACAGCTCCTTCCCTTCCCCAACAGGCTTGGAACCAA
GCTGCAAGTCCTGTGGGGCTCACTTTGCAAACACGGCCAGGAAGCAGACCTGCTTGGACTGTAAGAAA
AATTTTTGCATGACCTGTTCGAGCCAAGTAGGGAATGGGCCCCGCCTCTGCCTTCTCTGCCAACGGTT
TCGAGCTACAGCCTTTCAGCGAGAGGAGCTCATGAAGATGAAGGTGAAGGACTTGAGGGACTATCTCA
GCCTCCATGACATCTCTACCGAAATGTGCCGGGAGAAAGAAGAGCTGGTGCTCTTGGTCCTTGGCCAG
CAGCCTGTAATCTCCCAGGAGGACAGGACTCGTGCCTCCACCTTGTCCCCAGACTTTCCTGAGCAGCA
GGCCTTCCTGACCCAGCCTCACTCCAGCATGGTTCCACCTACCTCACCCAACCTCCCCTCTTCATCTG
CACAAGCCACCTCTGTTCCCCCAGCCCAGGTTCAGGAGAATCAGCAGGCCAATGGCCATGTGTCTCAG
GATCAAGAGGAACCCGTCTACCTGGAGAGCGTGGCCAGAGTACCTGCTGAGGATGAGACCCAGTCTAT
TGACTCAGAGGACAGCTTTGTCCCAGGCCGAAGGGCCTCTCTGTCTGACCTGACTGACCTGGAGGACA
TTGAAGGCCTGACAGTGCGGCAGCTGAAAGAGATCTTGGCTCGCAACTTTGTCAACTACAAGGGCTGC
TGTGAGAAGTGGGAGCTGATGGAGAGAGTGACCCGGCTATACAAGGATCAGAAAGGACTCCAGCACCT
GGAAGCCCTTCAGCGGCCAGTAGCATCTGACTTTGAGCCTCAGGGTCTGAGTGAAGCCGCTCGTTGGA
ACTCCAAGGAAAACCTTCTCGCTGGACCCAGTGAAAATGACCCCAACCTTTTCGTTGCACTGTATGAT
TTTGTGGCCAGTGGAGATAACACTCTAAGCATAACTAAAGGTGAAAAGCTCCGGGTCTTAGGCTATAA
TCACAATGGGGAATGGTGTGAAGCCCAAACCAAAAATGGCCAAGGCTGGGTCCCAAGCAACTACATCA
CGCCAGTCAACAGTCTGGAGAAACACTCCTGGTACCATGGGCCTGTGTCCCGCAATGCCGCTGAGTAT
CTGCTGAGCAGCGGGATCAATGGCAGCTTCTTGGTGCGTGAGAGTGAGAGCAGTCCTGGCCAGAGGTC
CATCTCGCTGAGATACGAAGGGAGGGTGTACCATTACAGGATCAACACTGCTTCTGATGGCAAGCTCT
ACGTCTCCTCCGAGAGCCGCTTCAACACCCTGGCCGAGTTGGTTCATCATCATTCAACGGTGGCCGAC
GGGCTCATCACCACGCTCCATTATCCAGCCCCAAAGCGCAACAAGCCCACTGTCTATGGTGTGTCCCC
CAACTACGACAAGTGGGAGATGGAACGCACGGACATCACCATGAAGCACAAGCTGGGCGGGGGCCAGT
ACGGGGAGGTGTACGAGGGCGTGTGGAAGAAATACAGCCTGACGGTGGCCGTGAAGACCTTGAAGGAG
GACACCATGGAGGTGGAAGAGTTCTTGAAAGAAGCTGCAGTCATGAAAGAGATCAAACACCCTAACCT
GGTGCAGCTCCTTGGGGTCTGCACCCGGGAGCCCCCGTTCTATATCATCACTGAGTTCATGACCTACG
GGAACCTCCTGGACTACCTGAGGGAGTGCAACCGGCAGGAGGTGAACGCCGTGGTGCTGCTGTACATG
GCCACTCAGATCTCGTCAGCCATGGAGTACCTGGAGAAGAAAAACTTCATCCACAGAGATCTTGCTGC
CCGAAACTGCCTGGTAGGGGAGAACCACTTGGTGAAGGTAGCTGATTTTGGCCTGAGCAGGTTGATGA
CAGGGGACACCTACACAGCCCATGCTGGAGCCAAGTTCCCCATCAAATGGACTGCACCCGAGAGCCTG
GCCTACAACAAGTTCTCCATCAAGTCCGACGTCTGGGCATTTGGAGTATTGCTTTGGGAAATTGCTAC
CTATGGCATGTCCCCTTACCCGGGAATTGACCTGTCCCAGGTGTATGAGCTGCTAGAGAAGGACTACC
GCATGGAGCGCCCAGAAGGCTGCCCAGAGAAGGTCTATGAACTCATGCGAGCATGTTGGCAGTGGAAT
CCCTCTGACCGGCCCTCCTTTGCTGAAATCCACCAAGCCTTTGAAACAATGTTCCAGGAATCCAGTAT
CTCAGACGAAGTGGAAAAGGAGCTGGGGAAACAAGGCGTCCGTGGGGCTGTGAGTACCTTGCTGCAGG
CCCCAGAGCTGCCCACCAAGACGAGGACCTCCAGGAGAGCTGCAGAGCACAGAGACACCACTGACGTG
CCTGAGATGCCTCACTCCAAGGGCCAGGGAGAGAGCGATCCTCTGGACCATGAGCCTGCCGTGTCTCC
ATTGCTCCCTCGAAAAGAGCGAGGTCCCCCGGAGGGCGGCCTGAATGAAGATGAGCGCCTTCTCCCCA
AAGACAAAAAGACCAACTTGTTCAGCGCCTTGATCAAGAAGAAGAAGAAGACAGCCCCAACCCCTCCC
AAACGCAGCAGCTCCTTCCGGGAGATGGACGGCCAGCCGGAGCGCAGAGGGGCCGGCGAGGAAGAGGG
CCGAGACATCAGCAACGGGGCACTGGCTTTCACCCCCTTGGACACAGCTGACCCAGCCAAGTCCCCAA
AGCCCAGCAATGGGGCTGGGGTCCCCAATGGAGCCCTCCGGGAGTCCGGGGGCTCAGGCTTCCGGTCT
CCCCACCTGTGGAAGAAGTCCAGCACGCTGACCAGCAGCCGCCTAGCCACCGGCGAGGAGGAGGGCGG
TGGCAGCTCCAGCAAGCGCTTCCTGCGCTCTTGCTCCGCCTCCTGCGTTCCCCATGGGGCCAAGGACA
CGGAGTGGAGGTCAGTCACGCTGCCTCGGGACTTGCAGTCCACGGGAAGACAGTTTGACTCGTCCACA
TTTGGAGGGCACAAAAGTGAGAAGCCGGCTCTGCCTCGGAAGAGGGCAGGGGAGAACAGGTCTGACCA
GGTGACCCGAGGCACAGTAACGCCTCCCCCCAGGCTGGTGAAAAAGAATGAGGAAGCTGCTGATGAGG
TCTTCAAAGACATCATGGAGTCCAGCCCGGGCTCCAGCCCGCCCAACCTGACTCCAAAACCCCTCCGG
CGGCAGGTCACCGTGGCCCCTGCCTCGGGCCTCCCCCACAAGGAAGAAGCTGGAAAGGGCAGTGCCTT
AGGGACCCCTGCTGCAGCTGAGCCAGTGACCCCCACCAGCAAAGCAGGCTCAGGTGCACCAGGGGGCA
CCAGCAAGGGCCCCGCCGAGGAGTCCAGAGTGAGGAGGCACAAGCACTCCTCTGAGTCGCCAGGGAGG
GACAAGGGGAAATTGTCCAGGCTCAAACCTGCCCCGCCGCCCCCACCAGCAGCCTCTGCAGGGAAGGC
TGGAGGAAAGCCCTCGCAGAGCCCGAGCCAGGAGGCGGCCGGGGAGGCAGTCCTGGGCGCAAAGACAA
AAGCCACGAGTCTGGTTGATGCTGTGAACAGTGACGCTGCCAAGCCCAGCCAGCCGGGAGAGGGCCTC
AAAAAGCCCGTGCTCCCGGCCACTCCAAAGCCACAGTCCGCCAAGCCGTCGGGGACCCCCATCAGCCC
AGCCCCCGTTCCCTCCACGTTGCCATCAGCATCCTCGGCCCTGGCAGGGGACCAGCCGTCTTCCACCG
CCTTCATCCCTCTCATATCAACCCGAGTGTCTCTTCGGAAAACCCGCCAGCCTCCAGAGCGGATCGCC
AGCGGCGCCATCACCAAGGGCGTGGTCCTGGACAGCACCGAGGCGCTGTGCCTCGCCATCTCTAGGAA
CTCCGAGCAGATGGCCAGCCACAGCGCAGTGCTGGAGGCCGGCAAAAACCTCTACACGTTCTGCGTGA
GCTATGTGGATTCCATCCAGCAAATGAGGAACAAGTTTGCCTTCCGAGAGGCCATCAACAAACTGGAG
AATAATCTCCGGGAGCTTCAGATCTGCCCGGCGACAGCAGGCAGTGGTCCAGCGGCCACTCAGGACTT
CAGCAAGCTCCTCAGTTCGGTGAAGGAAATCAGTGACATAGTGCAGAGGTAG

In some embodiments, an ABL1 fusion nucleic acid molecule of the disclosure is a SLC27A4-ABL1 fusion nucleic acid molecule, wherein the SLC27A4-ABL1 fusion nucleic acid molecule encodes an SLC27A4-ABL1 fusion polypeptide comprising the amino acid sequence of SEQ ID NO: 26, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 26. In some embodiments, the SLC27A4-ABL1 fusion nucleic acid molecule comprises the nucleotide sequence of SEQ ID NO: 5, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 5.

(SEQ ID NO: 5)
ATGCTGCTTGGAGCCTCTCTGGTGGGGGTGCTGCTGTTCTCCAAGCTGGTGCTGAAACTGCCCTGGAC
CCAGGTGGGATTCTCCCTGTTGTTCCTCTACTTGGGATCTGGCGGCTGGCGCTTCATCCGGGTCTTCA
TCAAGACCATCAGGCGCGATATCTTTGGCGGCCTGGTCCTCCTGAAGGTGAAGGCAAAGGTGCGACAG
TGCCTGCAGGAGCGGCGGACAGTGCCCATTTTGTTTGCCTCTACCGTTCGGCGCCACCCCGACAAGAC
GGCCCTGATCTTCGAGGGCACAGATACCCACTGGACCTTCCGCCAGCTGGATGAGTACTCAAGCAGTG
TAGCCAACTTCCTGCAGGCCCGGGGCCTGGCCTCGGGCGATGTGGCTGCCATCTTCATGGAGAACCGC
AATGAGTTCGTGGGCCTATGGCTGGGCATGGCCAAGCTCGGTGTGGAGGCAGCCCTCATCAACACCAA
CCTGCGGCGGGATGCTCTGCTCCACTGCCTCACCACCTCGCGCGCACGGGCCCTTGTCTTTGGCAGCG
AAATGGCCTCAGCCATCTGTGAGGTCCATGCCAGCCTGGACCCCTCGCTCAGCCTCTTCTGCTCTGGC
TCCTGGGAGCCCGGTGCGGTGCCTCCAAGCACAGAACACCTGGACCCTCTGCTGAAAGATGCTCCCAA
GCACCTTCCCAGTTGCCCTGACAAGGGCTTCACAGATAAACTGTTCTACATCTACACATCCGGCACCA
CAGGGCTGCCCAAGGCCGCCATCGTGGTGCACAGCAGGTATTACCGCATGGCTGCCCTGGTGTACTAT
GGATTCCGCATGCGGCCCAACGACATCGTCTATGACTGCCTCCCCCTCTACCACTCAGCAGGAAACAT
CGTGGGAATCGGCCAGTGCCTGCTGCATGGCATGACGGTGGTGATTCGGAAGAAGTTCTCAGCCTCCC
GGTTCTGGGACGATTGTATCAAGTACAACTGCACGATTGTGCAGTACATTGGTGAACTGTGCCGCTAC
CTCCTGAACCAGCCACCGCGGGAGGCAGAAAACCAGCACCAGGTTCGCATGGCACTAGGCAATGGCCT
CCGGCAGTCCATCTGGACCAACTTTTCCAGCCGCTTCCACATACCCCAGGTGGCTGAGTTCTACGGGG
CCACAGAGTGCAACTGTAGCCTGGGCAACTTCGACAGCCAGGTGGGGGCCTGTGGTTTCAATAGCCGC
ATCCTGTCCTTCGTGTACCCCATCCGGTTGGTACGTGTCAACGAGGACACCATGGAGCTGATCCGGGG
GCCCGACGGCGTCTGCATTCCCTGCCAGCCAGGTGAGCCGGGCCAGCTGGTGGGCCGCATCATCCAGA
AAGACCCCCTGCGCCGCTTCGATGGCTACCTCAACCAGGGCGCCAACAACAAGAAGATTGCCAAGGAT
GTCTTCAAGAAGGGGGACCAGGCCTACCTTACTGGTGATGTGCTGGTGATGGACGAGCTGGGCTACCT
GTACTTCCGAGACCGCACTGGGGACACGTTCCGCTGGAAAGGTGAGAACGTGTCCACCACCGAGGTGG
AAGGCACACTCAGCCGCCTGCTGGACATGGCTGACGTGGCCGTGTATGGTGTCGAGGTGCCAGGAACC
GAGGGCCGGGCCGGAATGGCTGCTGTGGCCAGCCCCACTGGCAACTGTGACCTGGAGCGCTTTGCTCA
GGTCTTGGAGAAGGAACTGCCCCTGTATGCGCGCCCCATCTTCCTGCGCCTCCTGCCTGAGCTGCACA
AAACAGGGGTCTGCACCCGGGAGCCCCCGTTCTATATCATCACTGAGTTCATGACCTACGGGAACCTC
CTGGACTACCTGAGGGAGTGCAACCGGCAGGAGGTGAACGCCGTGGTGCTGCTGTACATGGCCACTCA
GATCTCGTCAGCCATGGAGTACCTGGAGAAGAAAAACTTCATCCACAGAGATCTTGCTGCCCGAAACT
GCCTGGTAGGGGAGAACCACTTGGTGAAGGTAGCTGATTTTGGCCTGAGCAGGTTGATGACAGGGGAC
ACCTACACAGCCCATGCTGGAGCCAAGTTCCCCATCAAATGGACTGCACCCGAGAGCCTGGCCTACAA
CAAGTTCTCCATCAAGTCCGACGTCTGGGCATTTGGAGTATTGCTTTGGGAAATTGCTACCTATGGCA
TGTCCCCTTACCCGGGAATTGACCTGTCCCAGGTGTATGAGCTGCTAGAGAAGGACTACCGCATGGAG
CGCCCAGAAGGCTGCCCAGAGAAGGTCTATGAACTCATGCGAGCATGTTGGCAGTGGAATCCCTCTGA
CCGGCCCTCCTTTGCTGAAATCCACCAAGCCTTTGAAACAATGTTCCAGGAATCCAGTATCTCAGACG
AAGTGGAAAAGGAGCTGGGGAAACAAGGCGTCCGTGGGGCTGTGAGTACCTTGCTGCAGGCCCCAGAG
CTGCCCACCAAGACGAGGACCTCCAGGAGAGCTGCAGAGCACAGAGACACCACTGACGTGCCTGAGAT
GCCTCACTCCAAGGGCCAGGGAGAGAGCGATCCTCTGGACCATGAGCCTGCCGTGTCTCCATTGCTCC
CTCGAAAAGAGCGAGGTCCCCCGGAGGGCGGCCTGAATGAAGATGAGCGCCTTCTCCCCAAAGACAAA
AAGACCAACTTGTTCAGCGCCTTGATCAAGAAGAAGAAGAAGACAGCCCCAACCCCTCCCAAACGCAG
CAGCTCCTTCCGGGAGATGGACGGCCAGCCGGAGCGCAGAGGGGCCGGCGAGGAAGAGGGCCGAGACA
TCAGCAACGGGGCACTGGCTTTCACCCCCTTGGACACAGCTGACCCAGCCAAGTCCCCAAAGCCCAGC
AATGGGGCTGGGGTCCCCAATGGAGCCCTCCGGGAGTCCGGGGGCTCAGGCTTCCGGTCTCCCCACCT
GTGGAAGAAGTCCAGCACGCTGACCAGCAGCCGCCTAGCCACCGGCGAGGAGGAGGGCGGTGGCAGCT
CCAGCAAGCGCTTCCTGCGCTCTTGCTCCGCCTCCTGCGTTCCCCATGGGGCCAAGGACACGGAGTGG
AGGTCAGTCACGCTGCCTCGGGACTTGCAGTCCACGGGAAGACAGTTTGACTCGTCCACATTTGGAGG
GCACAAAAGTGAGAAGCCGGCTCTGCCTCGGAAGAGGGCAGGGGAGAACAGGTCTGACCAGGTGACCC
GAGGCACAGTAACGCCTCCCCCCAGGCTGGTGAAAAAGAATGAGGAAGCTGCTGATGAGGTCTTCAAA
GACATCATGGAGTCCAGCCCGGGCTCCAGCCCGCCCAACCTGACTCCAAAACCCCTCCGGCGGCAGGT
CACCGTGGCCCCTGCCTCGGGCCTCCCCCACAAGGAAGAAGCTGGAAAGGGCAGTGCCTTAGGGACCC
CTGCTGCAGCTGAGCCAGTGACCCCCACCAGCAAAGCAGGCTCAGGTGCACCAGGGGGCACCAGCAAG
GGCCCCGCCGAGGAGTCCAGAGTGAGGAGGCACAAGCACTCCTCTGAGTCGCCAGGGAGGGACAAGGG
GAAATTGTCCAGGCTCAAACCTGCCCCGCCGCCCCCACCAGCAGCCTCTGCAGGGAAGGCTGGAGGAA
AGCCCTCGCAGAGCCCGAGCCAGGAGGCGGCCGGGGAGGCAGTCCTGGGCGCAAAGACAAAAGCCACG
AGTCTGGTTGATGCTGTGAACAGTGACGCTGCCAAGCCCAGCCAGCCGGGAGAGGGCCTCAAAAAGCC
CGTGCTCCCGGCCACTCCAAAGCCACAGTCCGCCAAGCCGTCGGGGACCCCCATCAGCCCAGCCCCCG
TTCCCTCCACGTTGCCATCAGCATCCTCGGCCCTGGCAGGGGACCAGCCGTCTTCCACCGCCTTCATC
CCTCTCATATCAACCCGAGTGTCTCTTCGGAAAACCCGCCAGCCTCCAGAGCGGATCGCCAGCGGCGC
CATCACCAAGGGCGTGGTCCTGGACAGCACCGAGGCGCTGTGCCTCGCCATCTCTAGGAACTCCGAGC
AGATGGCCAGCCACAGCGCAGTGCTGGAGGCCGGCAAAAACCTCTACACGTTCTGCGTGAGCTATGTG
GATTCCATCCAGCAAATGAGGAACAAGTTTGCCTTCCGAGAGGCCATCAACAAACTGGAGAATAATCT
CCGGGAGCTTCAGATCTGCCCGGCGACAGCAGGCAGTGGTCCAGCGGCCACTCAGGACTTCAGCAAGC
TCCTCAGTTCGGTGAAGGAAATCAGTGACATAGTGCAGAGGTAG

In some embodiments, an ABL1 fusion nucleic acid molecule of the disclosure is a BCR-ABL1 fusion nucleic acid molecule, wherein the BCR-ABL1 fusion nucleic acid molecule encodes a BCR-ABL1 fusion polypeptide comprising the amino acid sequence of SEQ ID NO: 27, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 27. In some embodiments, the BCR-ABL1 fusion nucleic acid molecule comprises the nucleotide sequence of SEQ ID NO: 6, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 6.

(SEQ ID NO: 6)
ATGGTGGACCCGGTGGGCTTCGCGGAGGCGTGGAAGGCGCAGTTCCCGGACTCAGAGCCCCCGCGCAT
GGAGCTGCGCTCAGTGGGCGACATCGAGCAGGAGCTGGAGCGCTGCAAGGCCTCCATTCGGCGCCTGG
AGCAGGAGGTGAACCAGGAGCGCTTCCGCATGATCTACCTGCAGACGTTGCTGGCCAAGGAAAAGAAG
AGCTATGACCGGCAGCGATGGGGCTTCCGGCGCGCGGCGCAGGCCCCCGACGGCGCCTCCGAGCCCCG
AGCGTCCGCGTCGCGCCCGCAGCCAGCGCCCGCCGACGGAGCCGACCCGCCGCCCGCCGAGGAGCCCG
AGGCCCGGCCCGACGGCGAGGGTTCTCCGGGTAAGGCCAGGCCCGGGACCGCCCGCAGGCCCGGGGCA
GCCGCGTCGGGGGAACGGGACGACCGGGGACCCCCCGCCAGCGTGGCGGCGCTCAGGTCCAACTTCGA
GCGGATCCGCAAGGGCCATGGCCAGCCCGGGGCGGACGCCGAGAAGCCCTTCTACGTGAACGTCGAGT
TTCACCACGAGCGCGGCCTGGTGAAGGTCAACGACAAAGAGGTGTCGGACCGCATCAGCTCCCTGGGC
AGCCAGGCCATGCAGATGGAGCGCAAAAAGTCCCAGCACGGCGCGGGCTCGAGCGTGGGGGATGCATC
CAGGCCCCCTTACCGGGGACGCTCCTCGGAGAGCAGCTGCGGCGTCGACGGCGACTACGAGGACGCCG
AGTTGAACCCCCGCTTCCTGAAGGACAACCTGATCGACGCCAATGGCGGTAGCAGGCCCCCTTGGCCG
CCCCTGGAGTACCAGCCCTACCAGAGCATCTACGTCGGGGGCATGATGGAAGGGGAGGGCAAGGGCCC
GCTCCTGCGCAGCCAGAGCACCTCTGAGCAGGAGAAGCGCCTTACCTGGCCCCGCAGGTCCTACTCCC
CCCGGAGTTTTGAGGATTGCGGAGGCGGCTATACCCCGGACTGCAGCTCCAATGAGAACCTCACCTCC
AGCGAGGAGGACTTCTCCTCTGGCCAGTCCAGCCGCGTGTCCCCAAGCCCCACCACCTACCGCATGTT
CCGGGACAAAAGCCGCTCTCCCTCGCAGAACTCGCAACAGTCCTTCGACAGCAGCAGTCCCCCCACGC
CGCAGTGCCATAAGCGGCACCGGCACTGCCCGGTTGTCGTGTCCGAGGCCACCATCGTGGGCGTCCGC
AAGACCGGGCAGATCTGGCCCAACGATGGCGAGGGCGCCTTCCATGGAGACGCAGATGGCTCGTTCGG
AACACCACCTGGATACGGCTGCGCTGCAGACCGGGCAGAGGAGCAGCGCCGGCACCAAGATGGGCTGC
CCTACATTGATGACTCGCCCTCCTCATCGCCCCACCTCAGCAGCAAGGGCAGGGGCAGCCGGGATGCG
CTGGTCTCGGGAGCCCTGGAGTCCACTAAAGCGAGTGAGCTGGACTTGGAAAAGGGCTTGGAGATGAG
AAAATGGGTCCTGTCGGGAATCCTGGCTAGCGAGGAGACTTACCTGAGCCACCTGGAGGCACTGCTGC
TGCCCATGAAGCCTTTGAAAGCCGCTGCCACCACCTCTCAGCCGGTGCTGACGAGTCAGCAGATCGAG
ACCATCTTCTTCAAAGTGCCTGAGCTCTACGAGATCCACAAGGAGTTCTATGATGGGCTCTTCCCCCG
CGTGCAGCAGTGGAGCCACCAGCAGCGGGTGGGCGACCTCTTCCAGAAGCTGGCCAGCCAGCTGGGTG
TGTACCGGGCCTTCGTGGACAACTACGGAGTTGCCATGGAAATGGCTGAGAAGTGCTGTCAGGCCAAT
GCTCAGTTTGCAGAAATCTCCGAGAACCTGAGAGCCAGAAGCAACAAAGATGCCAAGGATCCAACGAC
CAAGAACTCTCTGGAAACTCTGCTCTACAAGCCTGTGGACCGTGTGACGAGGAGCACGCTGGTCCTCC
ATGACTTGCTGAAGCACACTCCTGCCAGCCACCCTGACCACCCCTTGCTGCAGGACGCCCTCCGCATC
TCACAGAACTTCCTGTCCAGCATCAATGAGGAGATCACACCCCGACGGCAGTCCATGACGGTGAAGAA
GGGAGAGCACCGGCAGCTGCTGAAGGACAGCTTCATGGTGGAGCTGGTGGAGGGGGCCCGCAAGCTGC
GCCACGTCTTCCTGTTCACCGACCTGCTTCTCTGCACCAAGCTCAAGAAGCAGAGCGGAGGCAAAACG
CAGCAGTATGACTGCAAATGGTACATTCCGCTCACGGATCTCAGCTTCCAGATGGTGGATGAACTGGA
GGCAGTGCCCAACATCCCCCTGGTGCCCGATGAGGAGCTGGACGCTTTGAAGATCAAGATCTCCCAGA
TCAAGAATGACATCCAGAGAGAGAAGAGGGCGAACAAGGGCAGCAAGGCTACGGAGAGGCTGAAGAAG
AAGCTGTCGGAGCAGGAGTCACTGCTGCTGCTTATGTCTCCCAGCATGGCCTTCAGGGTGCACAGCCG
CAACGGCAAGAGTTACACGTTCCTGATCTCCTCTGACTATGAGCGTGCAGAGTGGAGGGAGAACATCC
GGGAGCAGCAGAAGAAGTGTTTCAGAAGCTTCTCCCTGACATCCGTGGAGCTGCAGATGCTGACCAAC
TCGTGTGTGAAACTCCAGACTGTCCACAGCATTCCGCTGACCATCAATAAGGAAGAAGCCCTTCAGCG
GCCAGTAGCATCTGACTTTGAGCCTCAGGGTCTGAGTGAAGCCGCTCGTTGGAACTCCAAGGAAAACC
TTCTCGCTGGACCCAGTGAAAATGACCCCAACCTTTTCGTTGCACTGTATGATTTTGTGGCCAGTGGA
GATAACACTCTAAGCATAACTAAAGGTGAAAAGCTCCGGGTCTTAGGCTATAATCACAATGGGGAATG
GTGTGAAGCCCAAACCAAAAATGGCCAAGGCTGGGTCCCAAGCAACTACATCACGCCAGTCAACAGTC
TGGAGAAACACTCCTGGTACCATGGGCCTGTGTCCCGCAATGCCGCTGAGTATCTGCTGAGCAGCGGG
ATCAATGGCAGCTTCTTGGTGCGTGAGAGTGAGAGCAGTCCTGGCCAGAGGTCCATCTCGCTGAGATA
CGAAGGGAGGGTGTACCATTACAGGATCAACACTGCTTCTGATGGCAAGCTCTACGTCTCCTCCGAGA
GCCGCTTCAACACCCTGGCCGAGTTGGTTCATCATCATTCAACGGTGGCCGACGGGCTCATCACCACG
CTCCATTATCCAGCCCCAAAGCGCAACAAGCCCACTGTCTATGGTGTGTCCCCCAACTACGACAAGTG
GGAGATGGAACGCACGGACATCACCATGAAGCACAAGCTGGGCGGGGGCCAGTACGGGGAGGTGTACG
AGGGCGTGTGGAAGAAATACAGCCTGACGGTGGCCGTGAAGACCTTGAAGGAGGACACCATGGAGGTG
GAAGAGTTCTTGAAAGAAGCTGCAGTCATGAAAGAGATCAAACACCCTAACCTGGTGCAGCTCCTTGG
GGTCTGCACCCGGGAGCCCCCGTTCTATATCATCACTGAGTTCATGACCTACGGGAACCTCCTGGACT
ACCTGAGGGAGTGCAACCGGCAGGAGGTGAACGCCGTGGTGCTGCTGTACATGGCCACTCAGATCTCG
TCAGCCATGGAGTACCTGGAGAAGAAAAACTTCATCCACAGAGATCTTGCTGCCCGAAACTGCCTGGT
AGGGGAGAACCACTTGGTGAAGGTAGCTGATTTTGGCCTGAGCAGGTTGATGACAGGGGACACCTACA
CAGCCCATGCTGGAGCCAAGTTCCCCATCAAATGGACTGCACCCGAGAGCCTGGCCTACAACAAGTTC
TCCATCAAGTCCGACGTCTGGGCATTTGGAGTATTGCTTTGGGAAATTGCTACCTATGGCATGTCCCC
TTACCCGGGAATTGACCTGTCCCAGGTGTATGAGCTGCTAGAGAAGGACTACCGCATGGAGCGCCCAG
AAGGCTGCCCAGAGAAGGTCTATGAACTCATGCGAGCATGTTGGCAGTGGAATCCCTCTGACCGGCCC
TCCTTTGCTGAAATCCACCAAGCCTTTGAAACAATGTTCCAGGAATCCAGTATCTCAGACGAAGTGGA
AAAGGAGCTGGGGAAACAAGGCGTCCGTGGGGCTGTGAGTACCTTGCTGCAGGCCCCAGAGCTGCCCA
CCAAGACGAGGACCTCCAGGAGAGCTGCAGAGCACAGAGACACCACTGACGTGCCTGAGATGCCTCAC
TCCAAGGGCCAGGGAGAGAGCGATCCTCTGGACCATGAGCCTGCCGTGTCTCCATTGCTCCCTCGAAA
AGAGCGAGGTCCCCCGGAGGGCGGCCTGAATGAAGATGAGCGCCTTCTCCCCAAAGACAAAAAGACCA
ACTTGTTCAGCGCCTTGATCAAGAAGAAGAAGAAGACAGCCCCAACCCCTCCCAAACGCAGCAGCTCC
TTCCGGGAGATGGACGGCCAGCCGGAGCGCAGAGGGGCCGGCGAGGAAGAGGGCCGAGACATCAGCAA
CGGGGCACTGGCTTTCACCCCCTTGGACACAGCTGACCCAGCCAAGTCCCCAAAGCCCAGCAATGGGG
CTGGGGTCCCCAATGGAGCCCTCCGGGAGTCCGGGGGCTCAGGCTTCCGGTCTCCCCACCTGTGGAAG
AAGTCCAGCACGCTGACCAGCAGCCGCCTAGCCACCGGCGAGGAGGAGGGCGGTGGCAGCTCCAGCAA
GCGCTTCCTGCGCTCTTGCTCCGCCTCCTGCGTTCCCCATGGGGCCAAGGACACGGAGTGGAGGTCAG
TCACGCTGCCTCGGGACTTGCAGTCCACGGGAAGACAGTTTGACTCGTCCACATTTGGAGGGCACAAA
AGTGAGAAGCCGGCTCTGCCTCGGAAGAGGGCAGGGGAGAACAGGTCTGACCAGGTGACCCGAGGCAC
AGTAACGCCTCCCCCCAGGCTGGTGAAAAAGAATGAGGAAGCTGCTGATGAGGTCTTCAAAGACATCA
TGGAGTCCAGCCCGGGCTCCAGCCCGCCCAACCTGACTCCAAAACCCCTCCGGCGGCAGGTCACCGTG
GCCCCTGCCTCGGGCCTCCCCCACAAGGAAGAAGCTGGAAAGGGCAGTGCCTTAGGGACCCCTGCTGC
AGCTGAGCCAGTGACCCCCACCAGCAAAGCAGGCTCAGGTGCACCAGGGGGCACCAGCAAGGGCCCCG
CCGAGGAGTCCAGAGTGAGGAGGCACAAGCACTCCTCTGAGTCGCCAGGGAGGGACAAGGGGAAATTG
TCCAGGCTCAAACCTGCCCCGCCGCCCCCACCAGCAGCCTCTGCAGGGAAGGCTGGAGGAAAGCCCTC
GCAGAGCCCGAGCCAGGAGGCGGCCGGGGAGGCAGTCCTGGGCGCAAAGACAAAAGCCACGAGTCTGG
TTGATGCTGTGAACAGTGACGCTGCCAAGCCCAGCCAGCCGGGAGAGGGCCTCAAAAAGCCCGTGCTC
CCGGCCACTCCAAAGCCACAGTCCGCCAAGCCGTCGGGGACCCCCATCAGCCCAGCCCCCGTTCCCTC
CACGTTGCCATCAGCATCCTCGGCCCTGGCAGGGGACCAGCCGTCTTCCACCGCCTTCATCCCTCTCA
TATCAACCCGAGTGTCTCTTCGGAAAACCCGCCAGCCTCCAGAGCGGATCGCCAGCGGCGCCATCACC
AAGGGCGTGGTCCTGGACAGCACCGAGGCGCTGTGCCTCGCCATCTCTAGGAACTCCGAGCAGATGGC
CAGCCACAGCGCAGTGCTGGAGGCCGGCAAAAACCTCTACACGTTCTGCGTGAGCTATGTGGATTCCA
TCCAGCAAATGAGGAACAAGTTTGCCTTCCGAGAGGCCATCAACAAACTGGAGAATAATCTCCGGGAG
CTTCAGATCTGCCCGGCGACAGCAGGCAGTGGTCCAGCGGCCACTCAGGACTTCAGCAAGCTCCTCAG
TTCGGTGAAGGAAATCAGTGACATAGTGCAGAGGTAG

In some embodiments, an ABL1 fusion nucleic acid molecule of the disclosure is a BCR-ABL1 fusion nucleic acid molecule, wherein the BCR-ABL1 fusion nucleic acid molecule encodes a BCR-ABL1 fusion polypeptide comprising the amino acid sequence of SEQ ID NO: 28, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 28. In some embodiments, the BCR-ABL1 fusion nucleic acid molecule comprises the nucleotide sequence of SEQ ID NO: 7, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 7.

(SEQ ID NO: 7)
ATGGTGGACCCGGTGGGCTTCGCGGAGGCGTGGAAGGCGCAGTTCCCGGACTCAGAGCCCCCGCGCAT
GGAGCTGCGCTCAGTGGGCGACATCGAGCAGGAGCTGGAGCGCTGCAAGGCCTCCATTCGGCGCCTGG
AGCAGGAGGTGAACCAGGAGCGCTTCCGCATGATCTACCTGCAGACGTTGCTGGCCAAGGAAAAGAAG
AGCTATGACCGGCAGCGATGGGGCTTCCGGCGCGCGGCGCAGGCCCCCGACGGCGCCTCCGAGCCCCG
AGCGTCCGCGTCGCGCCCGCAGCCAGCGCCCGCCGACGGAGCCGACCCGCCGCCCGCCGAGGAGCCCG
AGGCCCGGCCCGACGGCGAGGGTTCTCCGGGTAAGGCCAGGCCCGGGACCGCCCGCAGGCCCGGGGCA
GCCGCGTCGGGGGAACGGGACGACCGGGGACCCCCCGCCAGCGTGGCGGCGCTCAGGTCCAACTTCGA
GCGGATCCGCAAGGGCCATGGCCAGCCCGGGGCGGACGCCGAGAAGCCCTTCTACGTGAACGTCGAGT
TTCACCACGAGCGCGGCCTGGTGAAGGTCAACGACAAAGAGGTGTCGGACCGCATCAGCTCCCTGGGC
AGCCAGGCCATGCAGATGGAGCGCAAAAAGTCCCAGCACGGCGCGGGCTCGAGCGTGGGGGATGCATC
CAGGCCCCCTTACCGGGGACGCTCCTCGGAGAGCAGCTGCGGCGTCGACGGCGACTACGAGGACGCCG
AGTTGAACCCCCGCTTCCTGAAGGACAACCTGATCGACGCCAATGGCGGTAGCAGGCCCCCTTGGCCG
CCCCTGGAGTACCAGCCCTACCAGAGCATCTACGTCGGGGGCATGATGGAAGGGGAGGGCAAGGGCCC
GCTCCTGCGCAGCCAGAGCACCTCTGAGCAGGAGAAGCGCCTTACCTGGCCCCGCAGGTCCTACTCCC
CCCGGAGTTTTGAGGATTGCGGAGGCGGCTATACCCCGGACTGCAGCTCCAATGAGAACCTCACCTCC
AGCGAGGAGGACTTCTCCTCTGGCCAGTCCAGCCGCGTGTCCCCAAGCCCCACCACCTACCGCATGTT
CCGGGACAAAAGCCGCTCTCCCTCGCAGAACTCGCAACAGTCCTTCGACAGCAGCAGTCCCCCCACGC
CGCAGTGCCATAAGCGGCACCGGCACTGCCCGGTTGTCGTGTCCGAGGCCACCATCGTGGGCGTCCGC
AAGACCGGGCAGATCTGGCCCAACGATGGCGAGGGCGCCTTCCATGGAGACGCAGATGGCTCGTTCGG
AACACCACCTGGATACGGCTGCGCTGCAGACCGGGCAGAGGAGCAGCGCCGGCACCAAGATGGGCTGC
CCTACATTGATGACTCGCCCTCCTCATCGCCCCACCTCAGCAGCAAGGGCAGGGGCAGCCGGGATGCG
CTGGTCTCGGGAGCCCTGGAGTCCACTAAAGCGAGTGAGCTGGACTTGGAAAAGGGCTTGGAGATGAG
AAAATGGGTCCTGTCGGGAATCCTGGCTAGCGAGGAGACTTACCTGAGCCACCTGGAGGCACTGCTGC
TGCCCATGAAGCCTTTGAAAGCCGCTGCCACCACCTCTCAGCCGGTGCTGACGAGTCAGCAGATCGAG
ACCATCTTCTTCAAAGTGCCTGAGCTCTACGAGATCCACAAGGAGTTCTATGATGGGCTCTTCCCCCG
CGTGCAGCAGTGGAGCCACCAGCAGCGGGTGGGCGACCTCTTCCAGAAGCTGGCCAGCCAGCTGGGTG
TGTACCGGGCCTTCGTGGACAACTACGGAGTTGCCATGGAAATGGCTGAGAAGTGCTGTCAGGCCAAT
GCTCAGTTTGCAGAAATCTCCGAGAACCTGAGAGCCAGAAGCAACAAAGATGCCAAGGATCCAACGAC
CAAGAACTCTCTGGAAACTCTGCTCTACAAGCCTGTGGACCGTGTGACGAGGAGCACGCTGGTCCTCC
ATGACTTGCTGAAGCACACTCCTGCCAGCCACCCTGACCACCCCTTGCTGCAGGACGCCCTCCGCATC
TCACAGAACTTCCTGTCCAGCATCAATGAGGAGATCACACCCCGACGGCAGTCCATGACGGTGAAGAA
GGGAGAGCACCGGCAGCTGCTGAAGGACAGCTTCATGGTGGAGCTGGTGGAGGGGGCCCGCAAGCTGC
GCCACGTCTTCCTGTTCACCGACCTGCTTCTCTGCACCAAGCTCAAGAAGCAGAGCGGAGGCAAAACG
CAGCAGTATGACTGCAAATGGTACATTCCGCTCACGGATCTCAGCTTCCAGATGGTGGATGAACTGGA
GGCAGTGCCCAACATCCCCCTGGTGCCCGATGAGGAGCTGGACGCTTTGAAGATCAAGATCTCCCAGA
TCAAGAATGACATCCAGAGAGAGAAGAGGGCGAACAAGGGCAGCAAGGCTACGGAGAGGCTGAAGAAG
AAGCTGTCGGAGCAGGAGTCACTGCTGCTGCTTATGTCTCCCAGCATGGCCTTCAGGGTGCACAGCCG
CAACGGCAAGAGTTACACGTTCCTGATCTCCTCTGACTATGAGCGTGCAGAGTGGAGGGAGAACATCC
GGGAGCAGCAGAAGAAGTGTTTCAGAAGCTTCTCCCTGACATCCGTGGAGCTGCAGATGCTGACCAAC
TCGTGTGTGAAACTCCAGACTGTCCACAGCATTCCGCTGACCATCAATAAGGAAGATGATGAGTCTCC
GGGGCTCTATGGGTTTCTGAATGTCATCGTCCACTCAGCCACTGGATTTAAGCAGAGTTCAAAAGCCC
TTCAGCGGCCAGTAGCATCTGACTTTGAGCCTCAGGGTCTGAGTGAAGCCGCTCGTTGGAACTCCAAG
GAAAACCTTCTCGCTGGACCCAGTGAAAATGACCCCAACCTTTTCGTTGCACTGTATGATTTTGTGGC
CAGTGGAGATAACACTCTAAGCATAACTAAAGGTGAAAAGCTCCGGGTCTTAGGCTATAATCACAATG
GGGAATGGTGTGAAGCCCAAACCAAAAATGGCCAAGGCTGGGTCCCAAGCAACTACATCACGCCAGTC
AACAGTCTGGAGAAACACTCCTGGTACCATGGGCCTGTGTCCCGCAATGCCGCTGAGTATCTGCTGAG
CAGCGGGATCAATGGCAGCTTCTTGGTGCGTGAGAGTGAGAGCAGTCCTGGCCAGAGGTCCATCTCGC
TGAGATACGAAGGGAGGGTGTACCATTACAGGATCAACACTGCTTCTGATGGCAAGCTCTACGTCTCC
TCCGAGAGCCGCTTCAACACCCTGGCCGAGTTGGTTCATCATCATTCAACGGTGGCCGACGGGCTCAT
CACCACGCTCCATTATCCAGCCCCAAAGCGCAACAAGCCCACTGTCTATGGTGTGTCCCCCAACTACG
ACAAGTGGGAGATGGAACGCACGGACATCACCATGAAGCACAAGCTGGGCGGGGGCCAGTACGGGGAG
GTGTACGAGGGCGTGTGGAAGAAATACAGCCTGACGGTGGCCGTGAAGACCTTGAAGGAGGACACCAT
GGAGGTGGAAGAGTTCTTGAAAGAAGCTGCAGTCATGAAAGAGATCAAACACCCTAACCTGGTGCAGC
TCCTTGGGGTCTGCACCCGGGAGCCCCCGTTCTATATCATCACTGAGTTCATGACCTACGGGAACCTC
CTGGACTACCTGAGGGAGTGCAACCGGCAGGAGGTGAACGCCGTGGTGCTGCTGTACATGGCCACTCA
GATCTCGTCAGCCATGGAGTACCTGGAGAAGAAAAACTTCATCCACAGAGATCTTGCTGCCCGAAACT
GCCTGGTAGGGGAGAACCACTTGGTGAAGGTAGCTGATTTTGGCCTGAGCAGGTTGATGACAGGGGAC
ACCTACACAGCCCATGCTGGAGCCAAGTTCCCCATCAAATGGACTGCACCCGAGAGCCTGGCCTACAA
CAAGTTCTCCATCAAGTCCGACGTCTGGGCATTTGGAGTATTGCTTTGGGAAATTGCTACCTATGGCA
TGTCCCCTTACCCGGGAATTGACCTGTCCCAGGTGTATGAGCTGCTAGAGAAGGACTACCGCATGGAG
CGCCCAGAAGGCTGCCCAGAGAAGGTCTATGAACTCATGCGAGCATGTTGGCAGTGGAATCCCTCTGA
CCGGCCCTCCTTTGCTGAAATCCACCAAGCCTTTGAAACAATGTTCCAGGAATCCAGTATCTCAGACG
AAGTGGAAAAGGAGCTGGGGAAACAAGGCGTCCGTGGGGCTGTGAGTACCTTGCTGCAGGCCCCAGAG
CTGCCCACCAAGACGAGGACCTCCAGGAGAGCTGCAGAGCACAGAGACACCACTGACGTGCCTGAGAT
GCCTCACTCCAAGGGCCAGGGAGAGAGCGATCCTCTGGACCATGAGCCTGCCGTGTCTCCATTGCTCC
CTCGAAAAGAGCGAGGTCCCCCGGAGGGCGGCCTGAATGAAGATGAGCGCCTTCTCCCCAAAGACAAA
AAGACCAACTTGTTCAGCGCCTTGATCAAGAAGAAGAAGAAGACAGCCCCAACCCCTCCCAAACGCAG
CAGCTCCTTCCGGGAGATGGACGGCCAGCCGGAGCGCAGAGGGGCCGGCGAGGAAGAGGGCCGAGACA
TCAGCAACGGGGCACTGGCTTTCACCCCCTTGGACACAGCTGACCCAGCCAAGTCCCCAAAGCCCAGC
AATGGGGCTGGGGTCCCCAATGGAGCCCTCCGGGAGTCCGGGGGCTCAGGCTTCCGGTCTCCCCACCT
GTGGAAGAAGTCCAGCACGCTGACCAGCAGCCGCCTAGCCACCGGCGAGGAGGAGGGCGGTGGCAGCT
CCAGCAAGCGCTTCCTGCGCTCTTGCTCCGCCTCCTGCGTTCCCCATGGGGCCAAGGACACGGAGTGG
AGGTCAGTCACGCTGCCTCGGGACTTGCAGTCCACGGGAAGACAGTTTGACTCGTCCACATTTGGAGG
GCACAAAAGTGAGAAGCCGGCTCTGCCTCGGAAGAGGGCAGGGGAGAACAGGTCTGACCAGGTGACCC
GAGGCACAGTAACGCCTCCCCCCAGGCTGGTGAAAAAGAATGAGGAAGCTGCTGATGAGGTCTTCAAA
GACATCATGGAGTCCAGCCCGGGCTCCAGCCCGCCCAACCTGACTCCAAAACCCCTCCGGCGGCAGGT
CACCGTGGCCCCTGCCTCGGGCCTCCCCCACAAGGAAGAAGCTGGAAAGGGCAGTGCCTTAGGGACCC
CTGCTGCAGCTGAGCCAGTGACCCCCACCAGCAAAGCAGGCTCAGGTGCACCAGGGGGCACCAGCAAG
GGCCCCGCCGAGGAGTCCAGAGTGAGGAGGCACAAGCACTCCTCTGAGTCGCCAGGGAGGGACAAGGG
GAAATTGTCCAGGCTCAAACCTGCCCCGCCGCCCCCACCAGCAGCCTCTGCAGGGAAGGCTGGAGGAA
AGCCCTCGCAGAGCCCGAGCCAGGAGGCGGCCGGGGAGGCAGTCCTGGGCGCAAAGACAAAAGCCACG
AGTCTGGTTGATGCTGTGAACAGTGACGCTGCCAAGCCCAGCCAGCCGGGAGAGGGCCTCAAAAAGCC
CGTGCTCCCGGCCACTCCAAAGCCACAGTCCGCCAAGCCGTCGGGGACCCCCATCAGCCCAGCCCCCG
TTCCCTCCACGTTGCCATCAGCATCCTCGGCCCTGGCAGGGGACCAGCCGTCTTCCACCGCCTTCATC
CCTCTCATATCAACCCGAGTGTCTCTTCGGAAAACCCGCCAGCCTCCAGAGCGGATCGCCAGCGGCGC
CATCACCAAGGGCGTGGTCCTGGACAGCACCGAGGCGCTGTGCCTCGCCATCTCTAGGAACTCCGAGC
AGATGGCCAGCCACAGCGCAGTGCTGGAGGCCGGCAAAAACCTCTACACGTTCTGCGTGAGCTATGTG
GATTCCATCCAGCAAATGAGGAACAAGTTTGCCTTCCGAGAGGCCATCAACAAACTGGAGAATAATCT
CCGGGAGCTTCAGATCTGCCCGGCGACAGCAGGCAGTGGTCCAGCGGCCACTCAGGACTTCAGCAAGC
TCCTCAGTTCGGTGAAGGAAATCAGTGACATAGTGCAGAGGTAG

In some embodiments, an ABL1 fusion nucleic acid molecule of the disclosure is a BCR-ABL1 fusion nucleic acid molecule, wherein the BCR-ABL1 fusion nucleic acid molecule encodes a BCR-ABL1 fusion polypeptide comprising the amino acid sequence of SEQ ID NO: 29, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 29. In some embodiments, the BCR-ABL1 fusion nucleic acid molecule comprises the nucleotide sequence of SEQ ID NO: 8, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 8.

(SEQ ID NO: 8)
ATGGTGGACCCGGTGGGCTTCGCGGAGGCGTGGAAGGCGCAGTTCCCGGACTCAGAGCCCCC
GCGCATGGAGCTGCGCTCAGTGGGCGACATCGAGCAGGAGCTGGAGCGCTGCAAGGCCTCCA
TTCGGCGCCTGGAGCAGGAGGTGAACCAGGAGCGCTTCCGCATGATCTACCTGCAGACGTTG
CTGGCCAAGGAAAAGAAGAGCTATGACCGGCAGCGATGGGGCTTCCGGCGCGCGGCGCAGGC
CCCCGACGGCGCCTCCGAGCCCCGAGCGTCCGCGTCGCGCCCGCAGCCAGCGCCCGCCGACG
GAGCCGACCCGCCGCCCGCCGAGGAGCCCGAGGCCCGGCCCGACGGCGAGGGTTCTCCGGGT
AAGGCCAGGCCCGGGACCGCCCGCAGGCCCGGGGCAGCCGCGTCGGGGGAACGGGACGACCG
GGGACCCCCCGCCAGCGTGGCGGCGCTCAGGTCCAACTTCGAGCGGATCCGCAAGGGCCATG
GCCAGCCCGGGGCGGACGCCGAGAAGCCCTTCTACGTGAACGTCGAGTTTCACCACGAGCGC
GGCCTGGTGAAGGTCAACGACAAAGAGGTGTCGGACCGCATCAGCTCCCTGGGCAGCCAGGC
CATGCAGATGGAGCGCAAAAAGTCCCAGCACGGCGCGGGCTCGAGCGTGGGGGATGCATCCA
GGCCCCCTTACCGGGGACGCTCCTCGGAGAGCAGCTGCGGCGTCGACGGCGACTACGAGGAC
GCCGAGTTGAACCCCCGCTTCCTGAAGGACAACCTGATCGACGCCAATGGCGGTAGCAGGCC
CCCTTGGCCGCCCCTGGAGTACCAGCCCTACCAGAGCATCTACGTCGGGGGCATGATGGAAG
GGGAGGGCAAGGGCCCGCTCCTGCGCAGCCAGAGCACCTCTGAGCAGGAGAAGCGCCTTACC
TGGCCCCGCAGGTCCTACTCCCCCCGGAGTTTTGAGGATTGCGGAGGCGGCTATACCCCGGA
CTGCAGCTCCAATGAGAACCTCACCTCCAGCGAGGAGGACTTCTCCTCTGGCCAGTCCAGCC
GCGTGTCCCCAAGCCCCACCACCTACCGCATGTTCCGGGACAAAAGCCGCTCTCCCTCGCAG
AACTCGCAACAGTCCTTCGACAGCAGCAGTCCCCCCACGCCGCAGTGCCATAAGCGGCACCG
GCACTGCCCGGTTGTCGTGTCCGAGGCCACCATCGTGGGCGTCCGCAAGACCGGGCAGATCT
GGCCCAACGATGGCGAGGGCGCCTTCCATGGAGACGCAGATGGCTCGTTCGGAACACCACCT
GGATACGGCTGCGCTGCAGACCGGGCAGAGGAGCAGCGCCGGCACCAAGATGGGCTGCCCTA
CATTGATGACTCGCCCTCCTCATCGCCCCACCTCAGCAGCAAGGGCAGGGGCAGCCGGGATG
CGCTGGTCTCGGGAGCCCTGGAGTCCACTAAAGCGAGTGAGCTGGACTTGGAAAAGGGCTTG
GAGATGAGAAAATGGGTCCTGTCGGGAATCCTGGCTAGCGAGGAGACTTACCTGAGCCACCT
GGAGGCACTGCTGCTGCCCATGAAGCCTTTGAAAGCCGCTGCCACCACCTCTCAGCCGGTGC
TGACGAGTCAGCAGATCGAGACCATCTTCTTCAAAGTGCCTGAGCTCTACGAGATCCACAAG
GAGTTCTATGATGGGCTCTTCCCCCGCGTGCAGCAGTGGAGCCACCAGCAGCGGGTGGGCGA
CCTCTTCCAGAAGCTGGCCAGCCAGCTGGGTGTGTACCGGGCCTTCGTGGACAACTACGGAG
TTGCCATGGAAATGGCTGAGAAGTGCTGTCAGGCCAATGCTCAGTTTGCAGAAATCTCCGAG
AACCTGAGAGCCAGAAGCAACAAAGATGCCAAGGATCCAACGACCAAGAACTCTCTGGAAAC
TCTGCTCTACAAGCCTGTGGACCGTGTGACGAGGAGCACGCTGGTCCTCCATGACTTGCTGA
AGCACACTCCTGCCAGCCACCCTGACCACCCCTTGCTGCAGGACGCCCTCCGCATCTCACAG
AACTTCCTGTCCAGCATCAATGAGGAGATCACACCCCGACGGCAGTCCATGACGGTGAAGAA
GGGAGAGCACCGGCAGCTGCTGAAGGACAGCTTCATGGTGGAGCTGGTGGAGGGGGCCCGCA
AGCTGCGCCACGTCTTCCTGTTCACCGACCTGCTTCTCTGCACCAAGCTCAAGAAGCAGAGC
GGAGGCAAAACGCAGCAGTATGACTGCAAATGGTACATTCCGCTCACGGATCTCAGCTTCCA
GATGGTGGATGAACTGGAGGCAGTGCCCAACATCCCCCTGGTGCCCGATGAGGAGCTGGACG
CTTTGAAGATCAAGATCTCCCAGATCAAGAATGACATCCAGAGAGAGAAGAGGGCGAACAAG
GGCAGCAAGGCTACGGAGAGGCTGAAGAAGAAGCTGTCGGAGCAGGAGTCACTGCTGCTGCT
TATGTCTCCCAGCATGGCCTTCAGGGTGCACAGCCGCAACGGCAAGAGTTACACGTTCCTGA
TCTCCTCTGACTATGAGCGTGCAGAGTGGAGGGAGAACATCCGGGAGCAGCAGAAGAAGTGT
TTCAGAAGCTTCTCCCTGACATCCGTGGAGCTGCAGATGCTGACCAACTCGTGTGTGAAACT
CCAGACTGTCCACAGCATTCCGCTGACCATCAATAAGGAAGATGATGAGTCTCCGGGGCTCT
ATGGGTTTCTGAATGTCATCGTCCACTCAGCCACTGGATTTAAGCAGAGTTCAAAAGCCCTT
CAGCGGCCAGTAGCATCTGACTTTGAGCCTCAGGGTCTGAGTGAAGCCGCTCGTTGGAACTC
CAAGGAAAACCTTCTCGCTGGACCCAGTGAAAATGACCCCAACCTTTTCGTTGCACTGTATG
ATTTTGTGGCCAGTGGAGATAACACTCTAAGCATAACTAAAGGTGAAAAGCTCCGGGTCTTA
GGCTATAATCACAATGGGGAATGGTGTGAAGCCCAAACCAAAAATGGCCAAGGCTGGGTCCC
AAGCAACTACATCACGCCAGTCAACAGTCTGGAGAAACACTCCTGGTACCATGGGCCTGTGT
CCCGCAATGCCGCTGAGTATCTGCTGAGCAGCGGGATCAATGGCAGCTTCTTGGTGCGTGAG
AGTGAGAGCAGTCCTGGCCAGAGGTCCATCTCGCTGAGATACGAAGGGAGGGTGTACCATTA
CAGGATCAACACTGCTTCTGATGGCAAGCTCTACGTCTCCTCCGAGAGCCGCTTCAACACCC
TGGCCGAGTTGGTTCATCATCATTCAACGGTGGCCGACGGGCTCATCACCACGCTCCATTAT
CCAGCCCCAAAGCGCAACAAGCCCACTGTCTATGGTGTGTCCCCCAACTACGACAAGTGGGA
GATGGAACGCACGGACATCACCATGAAGCACAAGCTGGGCGGGGGCCAGTACGGGGAGGTGT
ACGAGGGCGTGTGGAAGAAATACAGCCTGACGGTGGCCGTGAAGACCTTGAAGGAGGACACC
ATGGAGGTGGAAGAGTTCTTGAAAGAAGCTGCAGTCATGAAAGAGATCAAACACCCTAACCT
GGTGCAGCTCCTTGGGGTCTGCACCCGGGAGCCCCCGTTCTATATCATCACTGAGTTCATGA
CCTACGGGAACCTCCTGGACTACCTGAGGGAGTGCAACCGGCAGGAGGTGAACGCCGTGGTG
CTGCTGTACATGGCCACTCAGATCTCGTCAGCCATGGAGTACCTGGAGAAGAAAAACTTCAT
CCACAGAGATCTTGCTGCCCGAAACTGCCTGGTAGGGGAGAACCACTTGGTGAAGGTAGCTG
ATTTTGGCCTGAGCAGGTTGATGACAGGGGACACCTACACAGCCCATGCTGGAGCCAAGTTC
CCCATCAAATGGACTGCACCCGAGAGCCTGGCCTACAACAAGTTCTCCATCAAGTCCGACGT
CTGGGCATTTGGAGTATTGCTTTGGGAAATTGCTACCTATGGCATGTCCCCTTACCCGGGAA
TTGACCTGTCCCAGGTGTATGAGCTGCTAGAGAAGGACTACCGCATGGAGCGCCCAGAAGGC
TGCCCAGAGAAGGTCTATGAACTCATGCGAGCATGTTGGCAGTGGAATCCCTCTGACCGGCC
CTCCTTTGCTGAAATCCACCAAGCCTTTGAAACAATGTTCCAGGAATCCAGTATCTCAGACG
AAGTGGAAAAGGAGCTGGGGAAACAAGGCGTCCGTGGGGCTGTGAGTACCTTGCTGCAGGCC
CCAGAGCTGCCCACCAAGACGAGGACCTCCAGGAGAGCTGCAGAGCACAGAGACACCACTGA
CGTGCCTGAGATGCCTCACTCCAAGGGCCAGGGAGAGAGCGATCCTCTGGACCATGAGCCTG
CCGTGTCTCCATTGCTCCCTCGAAAAGAGCGAGGTCCCCCGGAGGGCGGCCTGAATGAAGAT
GAGCGCCTTCTCCCCAAAGACAAAAAGACCAACTTGTTCAGCGCCTTGATCAAGAAGAAGAA
GAAGACAGCCCCAACCCCTCCCAAACGCAGCAGCTCCTTCCGGGAGATGGACGGCCAGCCGG
AGCGCAGAGGGGCCGGCGAGGAAGAGGGCCGAGACATCAGCAACGGGGCACTGGCTTTCACC
CCCTTGGACACAGCTGACCCAGCCAAGTCCCCAAAGCCCAGCAATGGGGCTGGGGTCCCCAA
TGGAGCCCTCCGGGAGTCCGGGGGCTCAGGCTTCCGGTCTCCCCACCTGTGGAAGAAGTCCA
GCACGCTGACCAGCAGCCGCCTAGCCACCGGCGAGGAGGAGGGCGGTGGCAGCTCCAGCAAG
CGCTTCCTGCGCTCTTGCTCCGCCTCCTGCGTTCCCCATGGGGCCAAGGACACGGAGTGGAG
GTCAGTCACGCTGCCTCGGGACTTGCAGTCCACGGGAAGACAGTTTGACTCGTCCACATTTG
GAGGGCACAAAAGTGAGAAGCCGGCTCTGCCTCGGAAGAGGGCAGGGGAGAACAGGTCTGAC
CAGGTGACCCGAGGCACAGTAACGCCTCCCCCCAGGCTGGTGAAAAAGAATGAGGAAGCTGC
TGATGAGGTCTTCAAAGACATCATGGAGTCCAGCCCGGGCTCCAGCCCGCCCAACCTGACTC
CAAAACCCCTCCGGCGGCAGGTCACCGTGGCCCCTGCCTCGGGCCTCCCCCACAAGGAAGAA
GCTGGAAAGGGCAGTGCCTTAGGGACCCCTGCTGCAGCTGAGCCAGTGACCCCCACCAGCAA
AGCAGGCTCAGGTGCACCAGGGGGCACCAGCAAGGGCCCCGCCGAGGAGTCCAGAGTGAGGA
GGCACAAGCACTCCTCTGAGTCGCCAGGGAGGGACAAGGGGAAATTGTCCAGGCTCAAACCT
GCCCCGCCGCCCCCACCAGCAGCCTCTGCAGGGAAGGCTGGAGGAAAGCCCTCGCAGAGCCC
GAGCCAGGAGGCGGCCGGGGAGGCAGTCCTGGGCGCAAAGACAAAAGCCACGAGTCTGGTTG
ATGCTGTGAACAGTGACGCTGCCAAGCCCAGCCAGCCGGGAGAGGGCCTCAAAAAGCCCGTG
CTCCCGGCCACTCCAAAGCCACAGTCCGCCAAGCCGTCGGGGACCCCCATCAGCCCAGCCCC
CGTTCCCTCCACGTTGCCATCAGCATCCTCGGCCCTGGCAGGGGACCAGCCGTCTTCCACCG
CCTTCATCCCTCTCATATCAACCCGAGTGTCTCTTCGGAAAACCCGCCAGCCTCCAGAGCGG
ATCGCCAGCGGCGCCATCACCAAGGGCGTGGTCCTGGACAGCACCGAGGCGCTGTGCCTCGC
CATCTCTAGGAACTCCGAGCAGATGGCCAGCCACAGCGCAGTGCTGGAGGCCGGCAAAAACC
TCTACACGTTCTGCGTGAGCTATGTGGATTCCATCCAGCAAATGAGGAACAAGTTTGCCTTC
CGAGAGGCCATCAACAAACTGGAGAATAATCTCCGGGAGCTTCAGATCTGCCCGGCGACAGC
AGGCAGTGGTCCAGCGGCCACTCAGGACTTCAGCAAGCTCCTCAGTTCGGTGAAGGAAATCA
GTGACATAGTGCAGAGGTAG

In some embodiments, an ABL1 fusion nucleic acid molecule of the disclosure is a BCR-ABL1 fusion nucleic acid molecule, wherein the BCR-ABL1 fusion nucleic acid molecule encodes a BCR-ABL1 fusion polypeptide comprising the amino acid sequence of SEQ ID NO: 30, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 30. In some embodiments, the BCR-ABL1 fusion nucleic acid molecule comprises the nucleotide sequence of SEQ ID NO: 9, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 9.

(SEQ ID NO: 9)
ATGGTGGACCCGGTGGGCTTCGCGGAGGCGTGGAAGGCGCAGTTCCCGGACTCAGAGCCCCCGCGCAT
GGAGCTGCGCTCAGTGGGCGACATCGAGCAGGAGCTGGAGCGCTGCAAGGCCTCCATTCGGCGCCTGG
AGCAGGAGGTGAACCAGGAGCGCTTCCGCATGATCTACCTGCAGACGTTGCTGGCCAAGGAAAAGAAG
AGCTATGACCGGCAGCGATGGGGCTTCCGGCGCGCGGCGCAGGCCCCCGACGGCGCCTCCGAGCCCCG
AGCGTCCGCGTCGCGCCCGCAGCCAGCGCCCGCCGACGGAGCCGACCCGCCGCCCGCCGAGGAGCCCG
AGGCCCGGCCCGACGGCGAGGGTTCTCCGGGTAAGGCCAGGCCCGGGACCGCCCGCAGGCCCGGGGCA
GCCGCGTCGGGGGAACGGGACGACCGGGGACCCCCCGCCAGCGTGGCGGCGCTCAGGTCCAACTTCGA
GCGGATCCGCAAGGGCCATGGCCAGCCCGGGGCGGACGCCGAGAAGCCCTTCTACGTGAACGTCGAGT
TTCACCACGAGCGCGGCCTGGTGAAGGTCAACGACAAAGAGGTGTCGGACCGCATCAGCTCCCTGGGC
AGCCAGGCCATGCAGATGGAGCGCAAAAAGTCCCAGCACGGCGCGGGCTCGAGCGTGGGGGATGCATC
CAGGCCCCCTTACCGGGGACGCTCCTCGGAGAGCAGCTGCGGCGTCGACGGCGACTACGAGGACGCCG
AGTTGAACCCCCGCTTCCTGAAGGACAACCTGATCGACGCCAATGGCGGTAGCAGGCCCCCTTGGCCG
CCCCTGGAGTACCAGCCCTACCAGAGCATCTACGTCGGGGGCATGATGGAAGGGGAGGGCAAGGGCCC
GCTCCTGCGCAGCCAGAGCACCTCTGAGCAGGAGAAGCGCCTTACCTGGCCCCGCAGGTCCTACTCCC
CCCGGAGTTTTGAGGATTGCGGAGGCGGCTATACCCCGGACTGCAGCTCCAATGAGAACCTCACCTCC
AGCGAGGAGGACTTCTCCTCTGGCCAGTCCAGCCGCGTGTCCCCAAGCCCCACCACCTACCGCATGTT
CCGGGACAAAAGCCGCTCTCCCTCGCAGAACTCGCAACAGTCCTTCGACAGCAGCAGTCCCCCCACGC
CGCAGTGCCATAAGCGGCACCGGCACTGCCCGGTTGTCGTGTCCGAGGCCACCATCGTGGGCGTCCGC
AAGACCGGGCAGATCTGGCCCAACGATGGCGAGGGCGCCTTCCATGGAGACGCAGATGGCTCGTTCGG
AACACCACCTGGATACGGCTGCGCTGCAGACCGGGCAGAGGAGCAGCGCCGGCACCAAGATGGGCTGC
CCTACATTGATGACTCGCCCTCCTCATCGCCCCACCTCAGCAGCAAGGGCAGGGGCAGCCGGGATGCG
CTGGTCTCGGGAGCCCTGGAGTCCACTAAAGCGAGTGAGCTGGACTTGGAAAAGGGCTTGGAGATGAG
AAAATGGGTCCTGTCGGGAATCCTGGCTAGCGAGGAGACTTACCTGAGCCACCTGGAGGCACTGCTGC
TGCCCATGAAGCCTTTGAAAGCCGCTGCCACCACCTCTCAGCCGGTGCTGACGAGTCAGCAGATCGAG
ACCATCTTCTTCAAAGTGCCTGAGCTCTACGAGATCCACAAGGAGTTCTATGATGGGCTCTTCCCCCG
CGTGCAGCAGTGGAGCCACCAGCAGCGGGTGGGCGACCTCTTCCAGAAGCTGGCCAGCCAGCTGGGTG
TGTACCGGGCCTTCGTGGACAACTACGGAGTTGCCATGGAAATGGCTGAGAAGTGCTGTCAGGCCAAT
GCTCAGTTTGCAGAAATCTCCGAGAACCTGAGAGCCAGAAGCAACAAAGATGCCAAGGATCCAACGAC
CAAGAACTCTCTGGAAACTCTGCTCTACAAGCCTGTGGACCGTGTGACGAGGAGCACGCTGGTCCTCC
ATGACTTGCTGAAGCACACTCCTGCCAGCCACCCTGACCACCCCTTGCTGCAGGACGCCCTCCGCATC
TCACAGAACTTCCTGTCCAGCATCAATGAGGAGATCACACCCCGACGGCAGTCCATGACGGTGAAGAA
GGGAGAGCACCGGCAGCTGCTGAAGGACAGCTTCATGGTGGAGCTGGTGGAGGGGGCCCGCAAGCTGC
GCCACGTCTTCCTGTTCACCGACCTGCTTCTCTGCACCAAGCTCAAGAAGCAGAGCGGAGGCAAAACG
CAGCAGTATGACTGCAAATGGTACATTCCGCTCACGGATCTCAGCTTCCAGATGGTGGATGAACTGGA
GGCAGTGCCCAACATCCCCCTGGTGCCCGATGAGGAGCTGGACGCTTTGAAGATCAAGATCTCCCAGA
TCAAGAATGACATCCAGAGAGAGAAGAGGGCGAACAAGGGCAGCAAGGCTACGGAGAGGCTGAAGAAG
AAGCTGTCGGAGCAGGAGTCACTGCTGCTGCTTATGTCTCCCAGCATGGCCTTCAGGGTGCACAGCCG
CAACGGCAAGAGTTACACGTTCCTGATCTCCTCTGACTATGAGCGTGCAGAGTGGAGGGAGAACATCC
GGGAGCAGCAGAAGAAGTGTTTCAGAAGCTTCTCCCTGACATCCGTGGAGCTGCAGATGCTGACCAAC
TCGTGTGTGAAACTCCAGACTGTCCACAGCATTCCGCTGACCATCAATAAGGAAGATGATGAGTCTCC
GGGGCTCTATGGGTTTCTGAATGTCATCGTCCACTCAGCCACTGGATTTAAGCAGAGTTCAAAAGCCC
TTCAGCGGCCAGTAGCATCTGACTTTGAGCCTCAGGGTCTGAGTGAAGCCGCTCGTTGGAACTCCAAG
GAAAACCTTCTCGCTGGACCCAGTGAAAATGACCCCAACCTTTTCGTTGCACTGTATGATTTTGTGGC
CAGTGGAGATAACACTCTAAGCATAACTAAAGGTGAAAAGCTCCGGGTCTTAGGCTATAATCACAATG
GGGAATGGTGTGAAGCCCAAACCAAAAATGGCCAAGGCTGGGTCCCAAGCAACTACATCACGCCAGTC
AACAGTCTGGAGAAACACTCCTGGTACCATGGGCCTGTGTCCCGCAATGCCGCTGAGTATCTGCTGAG
CAGCGGGATCAATGGCAGCTTCTTGGTGCGTGAGAGTGAGAGCAGTCCTGGCCAGAGGTCCATCTCGC
TGAGATACGAAGGGAGGGTGTACCATTACAGGATCAACACTGCTTCTGATGGCAAGCTCTACGTCTCC
TCCGAGAGCCGCTTCAACACCCTGGCCGAGTTGGTTCATCATCATTCAACGGTGGCCGACGGGCTCAT
CACCACGCTCCATTATCCAGCCCCAAAGCGCAACAAGCCCACTGTCTATGGTGTGTCCCCCAACTACG
ACAAGTGGGAGATGGAACGCACGGACATCACCATGAAGCACAAGCTGGGGGGGGCCAGTACGGGGAG
GTGTACGAGGGCGTGTGGAAGAAATACAGCCTGACGGTGGCCGTGAAGACCTTGAAGGAGGACACCAT
GGAGGTGGAAGAGTTCTTGAAAGAAGCTGCAGTCATGAAAGAGATCAAACACCCTAACCTGGTGCAGC
TCCTTGGGGTCTGCACCCGGGAGCCCCCGTTCTATATCATCACTGAGTTCATGACCTACGGGAACCTC
CTGGACTACCTGAGGGAGTGCAACCGGCAGGAGGTGAACGCCGTGGTGCTGCTGTACATGGCCACTCA
GATCTCGTCAGCCATGGAGTACCTGGAGAAGAAAAACTTCATCCACAGAGATCTTGCTGCCCGAAACT
GCCTGGTAGGGGAGAACCACTTGGTGAAGGTAGCTGATTTTGGCCTGAGCAGGTTGATGACAGGGGAC
ACCTACACAGCCCATGCTGGAGCCAAGTTCCCCATCAAATGGACTGCACCCGAGAGCCTGGCCTACAA
CAAGTTCTCCATCAAGTCCGACGTCTGGGCATTTGGAGTATTGCTTTGGGAAATTGCTACCTATGGCA
TGTCCCCTTACCCGGGAATTGACCTGTCCCAGGTGTATGAGCTGCTAGAGAAGGACTACCGCATGGAG
CGCCCAGAAGGCTGCCCAGAGAAGGTCTATGAACTCATGCGAGCATGTTGGCAGTGGAATCCCTCTGA
CCGGCCCTCCTTTGCTGAAATCCACCAAGCCTTTGAAACAATGTTCCAGGAATCCAGTATCTCAGACG
AAGTGGAAAAGGAGCTGGGGAAACAAGGCGTCCGTGGGGCTGTGAGTACCTTGCTGCAGGCCCCAGAG
CTGCCCACCAAGACGAGGACCTCCAGGAGAGCTGCAGAGCACAGAGACACCACTGACGTGCCTGAGAT
GCCTCACTCCAAGGGCCAGGGAGAGAGCGATCCTCTGGACCATGAGCCTGCCGTGTCTCCATTGCTCC
CTCGAAAAGAGCGAGGTCCCCCGGAGGGCGGCCTGAATGAAGATGAGCGCCTTCTCCCCAAAGACAAA
AAGACCAACTTGTTCAGCGCCTTGATCAAGAAGAAGAAGAAGACAGCCCCAACCCCTCCCAAACGCAG
CAGCTCCTTCCGGGAGATGGACGGCCAGCCGGAGCGCAGAGGGGCCGGCGAGGAAGAGGGCCGAGACA
TCAGCAACGGGGCACTGGCTTTCACCCCCTTGGACACAGCTGACCCAGCCAAGTCCCCAAAGCCCAGC
AATGGGGCTGGGGTCCCCAATGGAGCCCTCCGGGAGTCCGGGGGCTCAGGCTTCCGGTCTCCCCACCT
GTGGAAGAAGTCCAGCACGCTGACCAGCAGCCGCCTAGCCACCGGCGAGGAGGAGGGCGGTGGCAGCT
CCAGCAAGCGCTTCCTGCGCTCTTGCTCCGCCTCCTGCGTTCCCCATGGGGCCAAGGACACGGAGTGG
AGGTCAGTCACGCTGCCTCGGGACTTGCAGTCCACGGGAAGACAGTTTGACTCGTCCACATTTGGAGG
GCACAAAAGTGAGAAGCCGGCTCTGCCTCGGAAGAGGGCAGGGGAGAACAGGTCTGACCAGGTGACCC
GAGGCACAGTAACGCCTCCCCCCAGGCTGGTGAAAAAGAATGAGGAAGCTGCTGATGAGGTCTTCAAA
GACATCATGGAGTCCAGCCCGGGCTCCAGCCCGCCCAACCTGACTCCAAAACCCCTCCGGCGGCAGGT
CACCGTGGCCCCTGCCTCGGGCCTCCCCCACAAGGAAGAAGCTGGAAAGGGCAGTGCCTTAGGGACCC
CTGCTGCAGCTGAGCCAGTGACCCCCACCAGCAAAGCAGGCTCAGGTGCACCAGGGGGCACCAGCAAG
GGCCCCGCCGAGGAGTCCAGAGTGAGGAGGCACAAGCACTCCTCTGAGTCGCCAGGGAGGGACAAGGG
GAAATTGTCCAGGCTCAAACCTGCCCCGCCGCCCCCACCAGCAGCCTCTGCAGGGAAGGCTGGAGGAA
AGCCCTCGCAGAGCCCGAGCCAGGAGGCGGCCGGGGAGGCAGTCCTGGGCGCAAAGACAAAAGCCACG
AGTCTGGTTGATGCTGTGAACAGTGACGCTGCCAAGCCCAGCCAGCCGGGAGAGGGCCTCAAAAAGCC
CGTGCTCCCGGCCACTCCAAAGCCACAGTCCGCCAAGCCGTCGGGGACCCCCATCAGCCCAGCCCCCG
TTCCCTCCACGTTGCCATCAGCATCCTCGGCCCTGGCAGGGGACCAGCCGTCTTCCACCGCCTTCATC
CCTCTCATATCAACCCGAGTGTCTCTTCGGAAAACCCGCCAGCCTCCAGAGCGGATCGCCAGCGGCGC
CATCACCAAGGGCGTGGTCCTGGACAGCACCGAGGCGCTGTGCCTCGCCATCTCTAGGAACTCCGAGC
AGATGGCCAGCCACAGCGCAGTGCTGGAGGCCGGCAAAAACCTCTACACGTTCTGCGTGAGCTATGTG
GATTCCATCCAGCAAATGAGGAACAAGTTTGCCTTCCGAGAGGCCATCAACAAACTGGAGAATAATCT
CCGGGAGCTTCAGATCTGCCCGGCGACAGCAGGCAGTGGTCCAGCGGCCACTCAGGACTTCAGCAAGC
TCCTCAGTTCGGTGAAGGAAATCAGTGACATAGTGCAGAGGTAG

In some embodiments, an ABL1 fusion nucleic acid molecule of the disclosure is a BCR-ABL1 fusion nucleic acid molecule, wherein the BCR-ABL1 fusion nucleic acid molecule encodes a BCR-ABL1 fusion polypeptide comprising the amino acid sequence of SEQ ID NO: 31, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 31. In some embodiments, the BCR-ABL1 fusion nucleic acid molecule comprises the nucleotide sequence of SEQ ID NO: 10, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 10.

(SEQ ID NO: 10)
ATGGTGGACCCGGTGGGCTTCGCGGAGGCGTGGAAGGCGCAGTTCCCGGACTCAGAGCCCCC
GCGCATGGAGCTGCGCTCAGTGGGCGACATCGAGCAGGAGCTGGAGCGCTGCAAGGCCTCCA
TTCGGCGCCTGGAGCAGGAGGTGAACCAGGAGCGCTTCCGCATGATCTACCTGCAGACGTTG
CTGGCCAAGGAAAAGAAGAGCTATGACCGGCAGCGATGGGGCTTCCGGCGCGCGGCGCAGGC
CCCCGACGGCGCCTCCGAGCCCCGAGCGTCCGCGTCGCGCCCGCAGCCAGCGCCCGCCGACG
GAGCCGACCCGCCGCCCGCCGAGGAGCCCGAGGCCCGGCCCGACGGCGAGGGTTCTCCGGGT
AAGGCCAGGCCCGGGACCGCCCGCAGGCCCGGGGCAGCCGCGTCGGGGGAACGGGACGACCG
GGGACCCCCCGCCAGCGTGGCGGCGCTCAGGTCCAACTTCGAGCGGATCCGCAAGGGCCATG
GCCAGCCCGGGGCGGACGCCGAGAAGCCCTTCTACGTGAACGTCGAGTTTCACCACGAGCGC
GGCCTGGTGAAGGTCAACGACAAAGAGGTGTCGGACCGCATCAGCTCCCTGGGCAGCCAGGC
CATGCAGATGGAGCGCAAAAAGTCCCAGCACGGCGCGGGCTCGAGCGTGGGGGATGCATCCA
GGCCCCCTTACCGGGGACGCTCCTCGGAGAGCAGCTGCGGCGTCGACGGCGACTACGAGGAC
GCCGAGTTGAACCCCCGCTTCCTGAAGGACAACCTGATCGACGCCAATGGCGGTAGCAGGCC
CCCTTGGCCGCCCCTGGAGTACCAGCCCTACCAGAGCATCTACGTCGGGGGCATGATGGAAG
GGGAGGGCAAGGGCCCGCTCCTGCGCAGCCAGAGCACCTCTGAGCAGGAGAAGCGCCTTACC
TGGCCCCGCAGGTCCTACTCCCCCCGGAGTTTTGAGGATTGCGGAGGCGGCTATACCCCGGA
CTGCAGCTCCAATGAGAACCTCACCTCCAGCGAGGAGGACTTCTCCTCTGGCCAGTCCAGCC
GCGTGTCCCCAAGCCCCACCACCTACCGCATGTTCCGGGACAAAAGCCGCTCTCCCTCGCAG
AACTCGCAACAGTCCTTCGACAGCAGCAGTCCCCCCACGCCGCAGTGCCATAAGCGGCACCG
GCACTGCCCGGTTGTCGTGTCCGAGGCCACCATCGTGGGCGTCCGCAAGACCGGGCAGATCT
GGCCCAACGATGGCGAGGGCGCCTTCCATGGAGACGCAGATGGCTCGTTCGGAACACCACCT
GGATACGGCTGCGCTGCAGACCGGGCAGAGGAGCAGCGCCGGCACCAAGATGGGCTGCCCTA
CATTGATGACTCGCCCTCCTCATCGCCCCACCTCAGCAGCAAGGGCAGGGGCAGCCGGGATG
CGCTGGTCTCGGGAGCCCTGGAGTCCACTAAAGCGAGTGAGCTGGACTTGGAAAAGGGCTTG
GAGATGAGAAAATGGGTCCTGTCGGGAATCCTGGCTAGCGAGGAGACTTACCTGAGCCACCT
GGAGGCACTGCTGCTGCCCATGAAGCCTTTGAAAGCCGCTGCCACCACCTCTCAGCCGGTGC
TGACGAGTCAGCAGATCGAGACCATCTTCTTCAAAGTGCCTGAGCTCTACGAGATCCACAAG
GAGTTCTATGATGGGCTCTTCCCCCGCGTGCAGCAGTGGAGCCACCAGCAGCGGGTGGGCGA
CCTCTTCCAGAAGCTGGCCAGCCAGCTGGGTGTGTACCGGGCCTTCGTGGACAACTACGGAG
TTGCCATGGAAATGGCTGAGAAGTGCTGTCAGGCCAATGCTCAGTTTGCAGAAATCTCCGAG
AACCTGAGAGCCAGAAGCAACAAAGATGCCAAGGATCCAACGACCAAGAACTCTCTGGAAAC
TCTGCTCTACAAGCCTGTGGACCGTGTGACGAGGAGCACGCTGGTCCTCCATGACTTGCTGA
AGCACACTCCTGCCAGCCACCCTGACCACCCCTTGCTGCAGGACGCCCTCCGCATCTCACAG
AACTTCCTGTCCAGCATCAATGAGGAGATCACACCCCGACGGCAGTCCATGACGGTGAAGAA
GGGAGAGCACCGGCAGCTGCTGAAGGACAGCTTCATGGTGGAGCTGGTGGAGGGGGCCCGCA
AGCTGCGCCACGTCTTCCTGTTCACCGACCTGCTTCTCTGCACCAAGCTCAAGAAGCAGAGC
GGAGGCAAAACGCAGCAGTATGACTGCAAATGGTACATTCCGCTCACGGATCTCAGCTTCCA
GATGGTGGATGAACTGGAGGCAGTGCCCAACATCCCCCTGGTGCCCGATGAGGAGCTGGACG
CTTTGAAGATCAAGATCTCCCAGATCAAGAATGACATCCAGAGAGAGAAGAGGGCGAACAAG
GGCAGCAAGGCTACGGAGAGGCTGAAGAAGAAGCTGTCGGAGCAGGAGTCACTGCTGCTGCT
TATGTCTCCCAGCATGGCCTTCAGGGTGCACAGCCGCAACGGCAAGAGTTACACGTTCCTGA
TCTCCTCTGACTATGAGCGTGCAGAGTGGAGGGAGAACATCCGGGAGCAGCAGAAGAAGTGT
TTCAGAAGCTTCTCCCTGACATCCGTGGAGCTGCAGATGCTGACCAACTCGTGTGTGAAACT
CCAGACTGTCCACAGCATTCCGCTGACCATCAATAAGGAAGATGATGAGTCTCCGGGGCTCT
ATGGGTTTCTGAATGTCATCGTCCACTCAGCCACTGGATTTAAGCAGAGTTCAAAAGCCCTT
CAGCGGCCAGTAGCATCTGACTTTGAGCCTCAGGGTCTGAGTGAAGCCGCTCGTTGGAACTC
CAAGGAAAACCTTCTCGCTGGACCCAGTGAAAATGACCCCAACCTTTTCGTTGCACTGTATG
ATTTTGTGGCCAGTGGAGATAACACTCTAAGCATAACTAAAGGTGAAAAGCTCCGGGTCTTA
GGCTATAATCACAATGGGGAATGGTGTGAAGCCCAAACCAAAAATGGCCAAGGCTGGGTCCC
AAGCAACTACATCACGCCAGTCAACAGTCTGGAGAAACACTCCTGGTACCATGGGCCTGTGT
CCCGCAATGCCGCTGAGTATCTGCTGAGCAGCGGGATCAATGGCAGCTTCTTGGTGCGTGAG
AGTGAGAGCAGTCCTGGCCAGAGGTCCATCTCGCTGAGATACGAAGGGAGGGTGTACCATTA
CAGGATCAACACTGCTTCTGATGGCAAGCTCTACGTCTCCTCCGAGAGCCGCTTCAACACCC
TGGCCGAGTTGGTTCATCATCATTCAACGGTGGCCGACGGGCTCATCACCACGCTCCATTAT
CCAGCCCCAAAGCGCAACAAGCCCACTGTCTATGGTGTGTCCCCCAACTACGACAAGTGGGA
GATGGAACGCACGGACATCACCATGAAGCACAAGCTGGGCGGGGGCCAGTACGGGGAGGTGT
ACGAGGGCGTGTGGAAGAAATACAGCCTGACGGTGGCCGTGAAGACCTTGAAGGAGGACACC
ATGGAGGTGGAAGAGTTCTTGAAAGAAGCTGCAGTCATGAAAGAGATCAAACACCCTAACCT
GGTGCAGCTCCTTGGGGTCTGCACCCGGGAGCCCCCGTTCTATATCATCACTGAGTTCATGA
CCTACGGGAACCTCCTGGACTACCTGAGGGAGTGCAACCGGCAGGAGGTGAACGCCGTGGTG
CTGCTGTACATGGCCACTCAGATCTCGTCAGCCATGGAGTACCTGGAGAAGAAAAACTTCAT
CCACAGAGATCTTGCTGCCCGAAACTGCCTGGTAGGGGAGAACCACTTGGTGAAGGTAGCTG
ATTTTGGCCTGAGCAGGTTGATGACAGGGGACACCTACACAGCCCATGCTGGAGCCAAGTTC
CCCATCAAATGGACTGCACCCGAGAGCCTGGCCTACAACAAGTTCTCCATCAAGTCCGACGT
CTGGGCATTTGGAGTATTGCTTTGGGAAATTGCTACCTATGGCATGTCCCCTTACCCGGGAA
TTGACCTGTCCCAGGTGTATGAGCTGCTAGAGAAGGACTACCGCATGGAGCGCCCAGAAGGC
TGCCCAGAGAAGGTCTATGAACTCATGCGAGCATGTTGGCAGTGGAATCCCTCTGACCGGCC
CTCCTTTGCTGAAATCCACCAAGCCTTTGAAACAATGTTCCAGGAATCCAGTATCTCAGACG
AAGTGGAAAAGGAGCTGGGGAAACAAGGCGTCCGTGGGGCTGTGAGTACCTTGCTGCAGGCC
CCAGAGCTGCCCACCAAGACGAGGACCTCCAGGAGAGCTGCAGAGCACAGAGACACCACTGA
CGTGCCTGAGATGCCTCACTCCAAGGGCCAGGGAGAGAGCGATCCTCTGGACCATGAGCCTG
CCGTGTCTCCATTGCTCCCTCGAAAAGAGCGAGGTCCCCCGGAGGGCGGCCTGAATGAAGAT
GAGCGCCTTCTCCCCAAAGACAAAAAGACCAACTTGTTCAGCGCCTTGATCAAGAAGAAGAA
GAAGACAGCCCCAACCCCTCCCAAACGCAGCAGCTCCTTCCGGGAGATGGACGGCCAGCCGG
AGCGCAGAGGGGCCGGCGAGGAAGAGGGCCGAGACATCAGCAACGGGGCACTGGCTTTCACC
CCCTTGGACACAGCTGACCCAGCCAAGTCCCCAAAGCCCAGCAATGGGGCTGGGGTCCCCAA
TGGAGCCCTCCGGGAGTCCGGGGGCTCAGGCTTCCGGTCTCCCCACCTGTGGAAGAAGTCCA
GCACGCTGACCAGCAGCCGCCTAGCCACCGGCGAGGAGGAGGGCGGTGGCAGCTCCAGCAAG
CGCTTCCTGCGCTCTTGCTCCGCCTCCTGCGTTCCCCATGGGGCCAAGGACACGGAGTGGAG
GTCAGTCACGCTGCCTCGGGACTTGCAGTCCACGGGAAGACAGTTTGACTCGTCCACATTTG
GAGGGCACAAAAGTGAGAAGCCGGCTCTGCCTCGGAAGAGGGCAGGGGAGAACAGGTCTGAC
CAGGTGACCCGAGGCACAGTAACGCCTCCCCCCAGGCTGGTGAAAAAGAATGAGGAAGCTGC
TGATGAGGTCTTCAAAGACATCATGGAGTCCAGCCCGGGCTCCAGCCCGCCCAACCTGACTC
CAAAACCCCTCCGGCGGCAGGTCACCGTGGCCCCTGCCTCGGGCCTCCCCCACAAGGAAGAA
GCTGGAAAGGGCAGTGCCTTAGGGACCCCTGCTGCAGCTGAGCCAGTGACCCCCACCAGCAA
AGCAGGCTCAGGTGCACCAGGGGGCACCAGCAAGGGCCCCGCCGAGGAGTCCAGAGTGAGGA
GGCACAAGCACTCCTCTGAGTCGCCAGGGAGGGACAAGGGGAAATTGTCCAGGCTCAAACCT
GCCCCGCCGCCCCCACCAGCAGCCTCTGCAGGGAAGGCTGGAGGAAAGCCCTCGCAGAGCCC
GAGCCAGGAGGCGGCCGGGGAGGCAGTCCTGGGCGCAAAGACAAAAGCCACGAGTCTGGTTG
ATGCTGTGAACAGTGACGCTGCCAAGCCCAGCCAGCCGGGAGAGGGCCTCAAAAAGCCCGTG
CTCCCGGCCACTCCAAAGCCACAGTCCGCCAAGCCGTCGGGGACCCCCATCAGCCCAGCCCC
CGTTCCCTCCACGTTGCCATCAGCATCCTCGGCCCTGGCAGGGGACCAGCCGTCTTCCACCG
CCTTCATCCCTCTCATATCAACCCGAGTGTCTCTTCGGAAAACCCGCCAGCCTCCAGAGCGG
ATCGCCAGCGGCGCCATCACCAAGGGCGTGGTCCTGGACAGCACCGAGGCGCTGTGCCTCGC
CATCTCTAGGAACTCCGAGCAGATGGCCAGCCACAGCGCAGTGCTGGAGGCCGGCAAAAACC
TCTACACGTTCTGCGTGAGCTATGTGGATTCCATCCAGCAAATGAGGAACAAGTTTGCCTTC
CGAGAGGCCATCAACAAACTGGAGAATAATCTCCGGGAGCTTCAGATCTGCCCGGCGACAGC
AGGCAGTGGTCCAGCGGCCACTCAGGACTTCAGCAAGCTCCTCAGTTCGGTGAAGGAAATCA
GTGACATAGTGCAGAGGTAG

In some embodiments, an ABL1 fusion nucleic acid molecule of the disclosure is a BCR-ABL1 fusion nucleic acid molecule, wherein the BCR-ABL1 fusion nucleic acid molecule encodes a BCR-ABL1 fusion polypeptide comprising the amino acid sequence of SEQ ID NO: 32, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 32. In some embodiments, the BCR-ABL1 fusion nucleic acid molecule comprises the nucleotide sequence of SEQ ID NO: 11, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 11.

(SEQ ID NO: 11)
ATGGTGGACCCGGTGGGCTTCGCGGAGGCGTGGAAGGCGCAGTTCCCGGACTCAGAGCCCCC
GCGCATGGAGCTGCGCTCAGTGGGCGACATCGAGCAGGAGCTGGAGCGCTGCAAGGCCTCCA
TTCGGCGCCTGGAGCAGGAGGTGAACCAGGAGCGCTTCCGCATGATCTACCTGCAGACGTTG
CTGGCCAAGGAAAAGAAGAGCTATGACCGGCAGCGATGGGGCTTCCGGCGCGCGGCGCAGGC
CCCCGACGGCGCCTCCGAGCCCCGAGCGTCCGCGTCGCGCCCGCAGCCAGCGCCCGCCGACG
GAGCCGACCCGCCGCCCGCCGAGGAGCCCGAGGCCCGGCCCGACGGCGAGGGTTCTCCGGGT
AAGGCCAGGCCCGGGACCGCCCGCAGGCCCGGGGCAGCCGCGTCGGGGGAACGGGACGACCG
GGGACCCCCCGCCAGCGTGGCGGCGCTCAGGTCCAACTTCGAGCGGATCCGCAAGGGCCATG
GCCAGCCCGGGGCGGACGCCGAGAAGCCCTTCTACGTGAACGTCGAGTTTCACCACGAGCGC
GGCCTGGTGAAGGTCAACGACAAAGAGGTGTCGGACCGCATCAGCTCCCTGGGCAGCCAGGC
CATGCAGATGGAGCGCAAAAAGTCCCAGCACGGCGCGGGCTCGAGCGTGGGGGATGCATCCA
GGCCCCCTTACCGGGGACGCTCCTCGGAGAGCAGCTGCGGCGTCGACGGCGACTACGAGGAC
GCCGAGTTGAACCCCCGCTTCCTGAAGGACAACCTGATCGACGCCAATGGCGGTAGCAGGCC
CCCTTGGCCGCCCCTGGAGTACCAGCCCTACCAGAGCATCTACGTCGGGGGCATGATGGAAG
GGGAGGGCAAGGGCCCGCTCCTGCGCAGCCAGAGCACCTCTGAGCAGGAGAAGCGCCTTACC
TGGCCCCGCAGGTCCTACTCCCCCCGGAGTTTTGAGGATTGCGGAGGCGGCTATACCCCGGA
CTGCAGCTCCAATGAGAACCTCACCTCCAGCGAGGAGGACTTCTCCTCTGGCCAGTCCAGCC
GCGTGTCCCCAAGCCCCACCACCTACCGCATGTTCCGGGACAAAAGCCGCTCTCCCTCGCAG
AACTCGCAACAGTCCTTCGACAGCAGCAGTCCCCCCACGCCGCAGTGCCATAAGCGGCACCG
GCACTGCCCGGTTGTCGTGTCCGAGGCCACCATCGTGGGCGTCCGCAAGACCGGGCAGATCT
GGCCCAACGATGGCGAGGGCGCCTTCCATGGAGACGCAGATGGCTCGTTCGGAACACCACCT
GGATACGGCTGCGCTGCAGACCGGGCAGAGGAGCAGCGCCGGCACCAAGATGGGCTGCCCTA
CATTGATGACTCGCCCTCCTCATCGCCCCACCTCAGCAGCAAGGGCAGGGGCAGCCGGGATG
CGCTGGTCTCGGGAGCCCTGGAGTCCACTAAAGCGAGTGAGCTGGACTTGGAAAAGGGCTTG
GAGATGAGAAAATGGGTCCTGTCGGGAATCCTGGCTAGCGAGGAGACTTACCTGAGCCACCT
GGAGGCACTGCTGCTGCCCATGAAGCCTTTGAAAGCCGCTGCCACCACCTCTCAGCCGGTGC
TGACGAGTCAGCAGATCGAGACCATCTTCTTCAAAGTGCCTGAGCTCTACGAGATCCACAAG
GAGTTCTATGATGGGCTCTTCCCCCGCGTGCAGCAGTGGAGCCACCAGCAGCGGGTGGGCGA
CCTCTTCCAGAAGCTGGCCAGCCAGCTGGGTGTGTACCGGGCCTTCGTGGACAACTACGGAG
TTGCCATGGAAATGGCTGAGAAGTGCTGTCAGGCCAATGCTCAGTTTGCAGAAATCTCCGAG
AACCTGAGAGCCAGAAGCAACAAAGATGCCAAGGATCCAACGACCAAGAACTCTCTGGAAAC
TCTGCTCTACAAGCCTGTGGACCGTGTGACGAGGAGCACGCTGGTCCTCCATGACTTGCTGA
AGCACACTCCTGCCAGCCACCCTGACCACCCCTTGCTGCAGGACGCCCTCCGCATCTCACAG
AACTTCCTGTCCAGCATCAATGAGGAGATCACACCCCGACGGCAGTCCATGACGGTGAAGAA
GGGAGAGCACCGGCAGCTGCTGAAGGACAGCTTCATGGTGGAGCTGGTGGAGGGGGCCCGCA
AGCTGCGCCACGTCTTCCTGTTCACCGACCTGCTTCTCTGCACCAAGCTCAAGAAGCAGAGC
GGAGGCAAAACGCAGCAGTATGACTGCAAATGGTACATTCCGCTCACGGATCTCAGCTTCCA
GATGGTGGATGAACTGGAGGCAGTGCCCAACATCCCCCTGGTGCCCGATGAGGAGCTGGACG
CTTTGAAGATCAAGATCTCCCAGATCAAGAATGACATCCAGAGAGAGAAGAGGGCGAACAAG
GGCAGCAAGGCTACGGAGAGGCTGAAGAAGAAGCTGTCGGAGCAGGAGTCACTGCTGCTGCT
TATGTCTCCCAGCATGGCCTTCAGGGTGCACAGCCGCAACGGCAAGAGTTACACGTTCCTGA
TCTCCTCTGACTATGAGCGTGCAGAGTGGAGGGAGAACATCCGGGAGCAGCAGAAGAAGTGT
TTCAGAAGCTTCTCCCTGACATCCGTGGAGCTGCAGATGCTGACCAACTCGTGTGTGAAACT
CCAGACTGTCCACAGCATTCCGCTGACCATCAATAAGGAAGATGATGAGTCTCCGGGGCTCT
ATGGGTTTCTGAATGTCATCGTCCACTCAGCCACTGGATTTAAGCAGAGTTCAAAAGCCCTT
CAGCGGCCAGTAGCATCTGACTTTGAGCCTCAGGGTCTGAGTGAAGCCGCTCGTTGGAACTC
CAAGGAAAACCTTCTCGCTGGACCCAGTGAAAATGACCCCAACCTTTTCGTTGCACTGTATG
ATTTTGTGGCCAGTGGAGATAACACTCTAAGCATAACTAAAGGTGAAAAGCTCCGGGTCTTA
GGCTATAATCACAATGGGGAATGGTGTGAAGCCCAAACCAAAAATGGCCAAGGCTGGGTCCC
AAGCAACTACATCACGCCAGTCAACAGTCTGGAGAAACACTCCTGGTACCATGGGCCTGTGT
CCCGCAATGCCGCTGAGTATCTGCTGAGCAGCGGGATCAATGGCAGCTTCTTGGTGCGTGAG
AGTGAGAGCAGTCCTGGCCAGAGGTCCATCTCGCTGAGATACGAAGGGAGGGTGTACCATTA
CAGGATCAACACTGCTTCTGATGGCAAGCTCTACGTCTCCTCCGAGAGCCGCTTCAACACCC
TGGCCGAGTTGGTTCATCATCATTCAACGGTGGCCGACGGGCTCATCACCACGCTCCATTAT
CCAGCCCCAAAGCGCAACAAGCCCACTGTCTATGGTGTGTCCCCCAACTACGACAAGTGGGA
GATGGAACGCACGGACATCACCATGAAGCACAAGCTGGGCGGGGGCCAGTACGGGGAGGTGT
ACGAGGGCGTGTGGAAGAAATACAGCCTGACGGTGGCCGTGAAGACCTTGAAGGAGGACACC
ATGGAGGTGGAAGAGTTCTTGAAAGAAGCTGCAGTCATGAAAGAGATCAAACACCCTAACCT
GGTGCAGCTCCTTGGGGTCTGCACCCGGGAGCCCCCGTTCTATATCATCACTGAGTTCATGA
CCTACGGGAACCTCCTGGACTACCTGAGGGAGTGCAACCGGCAGGAGGTGAACGCCGTGGTG
CTGCTGTACATGGCCACTCAGATCTCGTCAGCCATGGAGTACCTGGAGAAGAAAAACTTCAT
CCACAGAGATCTTGCTGCCCGAAACTGCCTGGTAGGGGAGAACCACTTGGTGAAGGTAGCTG
ATTTTGGCCTGAGCAGGTTGATGACAGGGGACACCTACACAGCCCATGCTGGAGCCAAGTTC
CCCATCAAATGGACTGCACCCGAGAGCCTGGCCTACAACAAGTTCTCCATCAAGTCCGACGT
CTGGGCATTTGGAGTATTGCTTTGGGAAATTGCTACCTATGGCATGTCCCCTTACCCGGGAA
TTGACCTGTCCCAGGTGTATGAGCTGCTAGAGAAGGACTACCGCATGGAGCGCCCAGAAGGC
TGCCCAGAGAAGGTCTATGAACTCATGCGAGCATGTTGGCAGTGGAATCCCTCTGACCGGCC
CTCCTTTGCTGAAATCCACCAAGCCTTTGAAACAATGTTCCAGGAATCCAGTATCTCAGACG
AAGTGGAAAAGGAGCTGGGGAAACAAGGCGTCCGTGGGGCTGTGAGTACCTTGCTGCAGGCC
CCAGAGCTGCCCACCAAGACGAGGACCTCCAGGAGAGCTGCAGAGCACAGAGACACCACTGA
CGTGCCTGAGATGCCTCACTCCAAGGGCCAGGGAGAGAGCGATCCTCTGGACCATGAGCCTG
CCGTGTCTCCATTGCTCCCTCGAAAAGAGCGAGGTCCCCCGGAGGGCGGCCTGAATGAAGAT
GAGCGCCTTCTCCCCAAAGACAAAAAGACCAACTTGTTCAGCGCCTTGATCAAGAAGAAGAA
GAAGACAGCCCCAACCCCTCCCAAACGCAGCAGCTCCTTCCGGGAGATGGACGGCCAGCCGG
AGCGCAGAGGGGCCGGCGAGGAAGAGGGCCGAGACATCAGCAACGGGGCACTGGCTTTCACC
CCCTTGGACACAGCTGACCCAGCCAAGTCCCCAAAGCCCAGCAATGGGGCTGGGGTCCCCAA
TGGAGCCCTCCGGGAGTCCGGGGGCTCAGGCTTCCGGTCTCCCCACCTGTGGAAGAAGTCCA
GCACGCTGACCAGCAGCCGCCTAGCCACCGGCGAGGAGGAGGGCGGTGGCAGCTCCAGCAAG
CGCTTCCTGCGCTCTTGCTCCGCCTCCTGCGTTCCCCATGGGGCCAAGGACACGGAGTGGAG
GTCAGTCACGCTGCCTCGGGACTTGCAGTCCACGGGAAGACAGTTTGACTCGTCCACATTTG
GAGGGCACAAAAGTGAGAAGCCGGCTCTGCCTCGGAAGAGGGCAGGGGAGAACAGGTCTGAC
CAGGTGACCCGAGGCACAGTAACGCCTCCCCCCAGGCTGGTGAAAAAGAATGAGGAAGCTGC
TGATGAGGTCTTCAAAGACATCATGGAGTCCAGCCCGGGCTCCAGCCCGCCCAACCTGACTC
CAAAACCCCTCCGGCGGCAGGTCACCGTGGCCCCTGCCTCGGGCCTCCCCCACAAGGAAGAA
GCTGGAAAGGGCAGTGCCTTAGGGACCCCTGCTGCAGCTGAGCCAGTGACCCCCACCAGCAA
AGCAGGCTCAGGTGCACCAGGGGGCACCAGCAAGGGCCCCGCCGAGGAGTCCAGAGTGAGGA
GGCACAAGCACTCCTCTGAGTCGCCAGGGAGGGACAAGGGGAAATTGTCCAGGCTCAAACCT
GCCCCGCCGCCCCCACCAGCAGCCTCTGCAGGGAAGGCTGGAGGAAAGCCCTCGCAGAGCCC
GAGCCAGGAGGCGGCCGGGGAGGCAGTCCTGGGCGCAAAGACAAAAGCCACGAGTCTGGTTG
ATGCTGTGAACAGTGACGCTGCCAAGCCCAGCCAGCCGGGAGAGGGCCTCAAAAAGCCCGTG
CTCCCGGCCACTCCAAAGCCACAGTCCGCCAAGCCGTCGGGGACCCCCATCAGCCCAGCCCC
CGTTCCCTCCACGTTGCCATCAGCATCCTCGGCCCTGGCAGGGGACCAGCCGTCTTCCACCG
CCTTCATCCCTCTCATATCAACCCGAGTGTCTCTTCGGAAAACCCGCCAGCCTCCAGAGCGG
ATCGCCAGCGGCGCCATCACCAAGGGCGTGGTCCTGGACAGCACCGAGGCGCTGTGCCTCGC
CATCTCTAGGAACTCCGAGCAGATGGCCAGCCACAGCGCAGTGCTGGAGGCCGGCAAAAACC
TCTACACGTTCTGCGTGAGCTATGTGGATTCCATCCAGCAAATGAGGAACAAGTTTGCCTTC
CGAGAGGCCATCAACAAACTGGAGAATAATCTCCGGGAGCTTCAGATCTGCCCGGCGACAGC
AGGCAGTGGTCCAGCGGCCACTCAGGACTTCAGCAAGCTCCTCAGTTCGGTGAAGGAAATCA
GTGACATAGTGCAGAGGTAG

In some embodiments, an ABL1 fusion nucleic acid molecule of the disclosure is a BCR-ABL1 fusion nucleic acid molecule, wherein the BCR-ABL1 fusion nucleic acid molecule encodes a BCR-ABL1 fusion polypeptide comprising the amino acid sequence of SEQ ID NO: 33, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 33. In some embodiments, the BCR-ABL1 fusion nucleic acid molecule comprises the nucleotide sequence of SEQ ID NO: 12, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 12.

(SEQ ID NO: 12)
ATGGTGGACCCGGTGGGCTTCGCGGAGGCGTGGAAGGCGCAGTTCCCGGACTCAGAGCCCCCGCGCAT
GGAGCTGCGCTCAGTGGGCGACATCGAGCAGGAGCTGGAGCGCTGCAAGGCCTCCATTCGGCGCCTGG
AGCAGGAGGTGAACCAGGAGCGCTTCCGCATGATCTACCTGCAGACGTTGCTGGCCAAGGAAAAGAAG
AGCTATGACCGGCAGCGATGGGGCTTCCGGCGCGCGGCGCAGGCCCCCGACGGCGCCTCCGAGCCCCG
AGCGTCCGCGTCGCGCCCGCAGCCAGCGCCCGCCGACGGAGCCGACCCGCCGCCCGCCGAGGAGCCCG
AGGCCCGGCCCGACGGCGAGGGTTCTCCGGGTAAGGCCAGGCCCGGGACCGCCCGCAGGCCCGGGGCA
GCCGCGTCGGGGGAACGGGACGACCGGGGACCCCCCGCCAGCGTGGCGGCGCTCAGGTCCAACTTCGA
GCGGATCCGCAAGGGCCATGGCCAGCCCGGGGCGGACGCCGAGAAGCCCTTCTACGTGAACGTCGAGT
TTCACCACGAGCGCGGCCTGGTGAAGGTCAACGACAAAGAGGTGTCGGACCGCATCAGCTCCCTGGGC
AGCCAGGCCATGCAGATGGAGCGCAAAAAGTCCCAGCACGGCGCGGGCTCGAGCGTGGGGGATGCATC
CAGGCCCCCTTACCGGGGACGCTCCTCGGAGAGCAGCTGCGGCGTCGACGGCGACTACGAGGACGCCG
AGTTGAACCCCCGCTTCCTGAAGGACAACCTGATCGACGCCAATGGCGGTAGCAGGCCCCCTTGGCCG
CCCCTGGAGTACCAGCCCTACCAGAGCATCTACGTCGGGGGCATGATGGAAGGGGAGGGCAAGGGCCC
GCTCCTGCGCAGCCAGAGCACCTCTGAGCAGGAGAAGCGCCTTACCTGGCCCCGCAGGTCCTACTCCC
CCCGGAGTTTTGAGGATTGCGGAGGCGGCTATACCCCGGACTGCAGCTCCAATGAGAACCTCACCTCC
AGCGAGGAGGACTTCTCCTCTGGCCAGTCCAGCCGCGTGTCCCCAAGCCCCACCACCTACCGCATGTT
CCGGGACAAAAGCCGCTCTCCCTCGCAGAACTCGCAACAGTCCTTCGACAGCAGCAGTCCCCCCACGC
CGCAGTGCCATAAGCGGCACCGGCACTGCCCGGTTGTCGTGTCCGAGGCCACCATCGTGGGCGTCCGC
AAGACCGGGCAGATCTGGCCCAACGATGGCGAGGGCGCCTTCCATGGAGACGCAGATGGCTCGTTCGG
AACACCACCTGGATACGGCTGCGCTGCAGACCGGGCAGAGGAGCAGCGCCGGCACCAAGATGGGCTGC
CCTACATTGATGACTCGCCCTCCTCATCGCCCCACCTCAGCAGCAAGGGCAGGGGCAGCCGGGATGCG
CTGGTCTCGGGAGCCCTGGAGTCCACTAAAGCGAGTGAGCTGGACTTGGAAAAGGGCTTGGAGATGAG
AAAATGGGTCCTGTCGGGAATCCTGGCTAGCGAGGAGACTTACCTGAGCCACCTGGAGGCACTGCTGC
TGCCCATGAAGCCTTTGAAAGCCGCTGCCACCACCTCTCAGCCGGTGCTGACGAGTCAGCAGATCGAG
ACCATCTTCTTCAAAGTGCCTGAGCTCTACGAGATCCACAAGGAGTTCTATGATGGGCTCTTCCCCCG
CGTGCAGCAGTGGAGCCACCAGCAGCGGGTGGGCGACCTCTTCCAGAAGCTGGCCAGCCAGCTGGGTG
TGTACCGGGCCTTCGTGGACAACTACGGAGTTGCCATGGAAATGGCTGAGAAGTGCTGTCAGGCCAAT
GCTCAGTTTGCAGAAATCTCCGAGAACCTGAGAGCCAGAAGCAACAAAGATGCCAAGGATCCAACGAC
CAAGAACTCTCTGGAAACTCTGCTCTACAAGCCTGTGGACCGTGTGACGAGGAGCACGCTGGTCCTCC
ATGACTTGCTGAAGCACACTCCTGCCAGCCACCCTGACCACCCCTTGCTGCAGGACGCCCTCCGCATC
TCACAGAACTTCCTGTCCAGCATCAATGAGGAGATCACACCCCGACGGCAGTCCATGACGGTGAAGAA
GGGAGAGCACCGGCAGCTGCTGAAGGACAGCTTCATGGTGGAGCTGGTGGAGGGGGCCCGCAAGCTGC
GCCACGTCTTCCTGTTCACCGACCTGCTTCTCTGCACCAAGCTCAAGAAGCAGAGCGGAGGCAAAACG
CAGCAGTATGACTGCAAATGGTACATTCCGCTCACGGATCTCAGCTTCCAGATGGTGGATGAACTGGA
GGCAGTGCCCAACATCCCCCTGGTGCCCGATGAGGAGCTGGACGCTTTGAAGATCAAGATCTCCCAGA
TCAAGAATGACATCCAGAGAGAGAAGAGGGCGAACAAGGGCAGCAAGGCTACGGAGAGGCTGAAGAAG
AAGCTGTCGGAGCAGGAGTCACTGCTGCTGCTTATGTCTCCCAGCATGGCCTTCAGGGTGCACAGCCG
CAACGGCAAGAGTTACACGTTCCTGATCTCCTCTGACTATGAGCGTGCAGAGTGGAGGGAGAACATCC
GGGAGCAGCAGAAGAAGTGTTTCAGAAGCTTCTCCCTGACATCCGTGGAGCTGCAGATGCTGACCAAC
TCGTGTGTGAAACTCCAGACTGTCCACAGCATTCCGCTGACCATCAATAAGGAAGATGATGAGTCTCC
GGGGCTCTATGGGTTTCTGAATGTCATCGTCCACTCAGCCACTGGATTTAAGCAGAGTTCAAAAGCCC
TTCAGCGGCCAGTAGCATCTGACTTTGAGCCTCAGGGTCTGAGTGAAGCCGCTCGTTGGAACTCCAAG
GAAAACCTTCTCGCTGGACCCAGTGAAAATGACCCCAACCTTTTCGTTGCACTGTATGATTTTGTGGC
CAGTGGAGATAACACTCTAAGCATAACTAAAGGTGAAAAGCTCCGGGTCTTAGGCTATAATCACAATG
GGGAATGGTGTGAAGCCCAAACCAAAAATGGCCAAGGCTGGGTCCCAAGCAACTACATCACGCCAGTC
AACAGTCTGGAGAAACACTCCTGGTACCATGGGCCTGTGTCCCGCAATGCCGCTGAGTATCTGCTGAG
CAGCGGGATCAATGGCAGCTTCTTGGTGCGTGAGAGTGAGAGCAGTCCTGGCCAGAGGTCCATCTCGC
TGAGATACGAAGGGAGGGTGTACCATTACAGGATCAACACTGCTTCTGATGGCAAGCTCTACGTCTCC
TCCGAGAGCCGCTTCAACACCCTGGCCGAGTTGGTTCATCATCATTCAACGGTGGCCGACGGGCTCAT
CACCACGCTCCATTATCCAGCCCCAAAGCGCAACAAGCCCACTGTCTATGGTGTGTCCCCCAACTACG
ACAAGTGGGAGATGGAACGCACGGACATCACCATGAAGCACAAGCTGGGCGGGGGCCAGTACGGGGAG
GTGTACGAGGGCGTGTGGAAGAAATACAGCCTGACGGTGGCCGTGAAGACCTTGAAGGAGGACACCAT
GGAGGTGGAAGAGTTCTTGAAAGAAGCTGCAGTCATGAAAGAGATCAAACACCCTAACCTGGTGCAGC
TCCTTGGGGTCTGCACCCGGGAGCCCCCGTTCTATATCATCACTGAGTTCATGACCTACGGGAACCTC
CTGGACTACCTGAGGGAGTGCAACCGGCAGGAGGTGAACGCCGTGGTGCTGCTGTACATGGCCACTCA
GATCTCGTCAGCCATGGAGTACCTGGAGAAGAAAAACTTCATCCACAGAGATCTTGCTGCCCGAAACT
GCCTGGTAGGGGAGAACCACTTGGTGAAGGTAGCTGATTTTGGCCTGAGCAGGTTGATGACAGGGGAC
ACCTACACAGCCCATGCTGGAGCCAAGTTCCCCATCAAATGGACTGCACCCGAGAGCCTGGCCTACAA
CAAGTTCTCCATCAAGTCCGACGTCTGGGCATTTGGAGTATTGCTTTGGGAAATTGCTACCTATGGCA
TGTCCCCTTACCCGGGAATTGACCTGTCCCAGGTGTATGAGCTGCTAGAGAAGGACTACCGCATGGAG
CGCCCAGAAGGCTGCCCAGAGAAGGTCTATGAACTCATGCGAGCATGTTGGCAGTGGAATCCCTCTGA
CCGGCCCTCCTTTGCTGAAATCCACCAAGCCTTTGAAACAATGTTCCAGGAATCCAGTATCTCAGACG
AAGTGGAAAAGGAGCTGGGGAAACAAGGCGTCCGTGGGGCTGTGAGTACCTTGCTGCAGGCCCCAGAG
CTGCCCACCAAGACGAGGACCTCCAGGAGAGCTGCAGAGCACAGAGACACCACTGACGTGCCTGAGAT
GCCTCACTCCAAGGGCCAGGGAGAGAGCGATCCTCTGGACCATGAGCCTGCCGTGTCTCCATTGCTCC
CTCGAAAAGAGCGAGGTCCCCCGGAGGGCGGCCTGAATGAAGATGAGCGCCTTCTCCCCAAAGACAAA
AAGACCAACTTGTTCAGCGCCTTGATCAAGAAGAAGAAGAAGACAGCCCCAACCCCTCCCAAACGCAG
CAGCTCCTTCCGGGAGATGGACGGCCAGCCGGAGCGCAGAGGGGCCGGCGAGGAAGAGGGCCGAGACA
TCAGCAACGGGGCACTGGCTTTCACCCCCTTGGACACAGCTGACCCAGCCAAGTCCCCAAAGCCCAGC
AATGGGGCTGGGGTCCCCAATGGAGCCCTCCGGGAGTCCGGGGGCTCAGGCTTCCGGTCTCCCCACCT
GTGGAAGAAGTCCAGCACGCTGACCAGCAGCCGCCTAGCCACCGGCGAGGAGGAGGGCGGTGGCAGCT
CCAGCAAGCGCTTCCTGCGCTCTTGCTCCGCCTCCTGCGTTCCCCATGGGGCCAAGGACACGGAGTGG
AGGTCAGTCACGCTGCCTCGGGACTTGCAGTCCACGGGAAGACAGTTTGACTCGTCCACATTTGGAGG
GCACAAAAGTGAGAAGCCGGCTCTGCCTCGGAAGAGGGCAGGGGAGAACAGGTCTGACCAGGTGACCC
GAGGCACAGTAACGCCTCCCCCCAGGCTGGTGAAAAAGAATGAGGAAGCTGCTGATGAGGTCTTCAAA
GACATCATGGAGTCCAGCCCGGGCTCCAGCCCGCCCAACCTGACTCCAAAACCCCTCCGGCGGCAGGT
CACCGTGGCCCCTGCCTCGGGCCTCCCCCACAAGGAAGAAGCTGGAAAGGGCAGTGCCTTAGGGACCC
CTGCTGCAGCTGAGCCAGTGACCCCCACCAGCAAAGCAGGCTCAGGTGCACCAGGGGGCACCAGCAAG
GGCCCCGCCGAGGAGTCCAGAGTGAGGAGGCACAAGCACTCCTCTGAGTCGCCAGGGAGGGACAAGGG
GAAATTGTCCAGGCTCAAACCTGCCCCGCCGCCCCCACCAGCAGCCTCTGCAGGGAAGGCTGGAGGAA
AGCCCTCGCAGAGCCCGAGCCAGGAGGCGGCCGGGGAGGCAGTCCTGGGCGCAAAGACAAAAGCCACG
AGTCTGGTTGATGCTGTGAACAGTGACGCTGCCAAGCCCAGCCAGCCGGGAGAGGGCCTCAAAAAGCC
CGTGCTCCCGGCCACTCCAAAGCCACAGTCCGCCAAGCCGTCGGGGACCCCCATCAGCCCAGCCCCCG
TTCCCTCCACGTTGCCATCAGCATCCTCGGCCCTGGCAGGGGACCAGCCGTCTTCCACCGCCTTCATC
CCTCTCATATCAACCCGAGTGTCTCTTCGGAAAACCCGCCAGCCTCCAGAGCGGATCGCCAGCGGCGC
CATCACCAAGGGCGTGGTCCTGGACAGCACCGAGGCGCTGTGCCTCGCCATCTCTAGGAACTCCGAGC
AGATGGCCAGCCACAGCGCAGTGCTGGAGGCCGGCAAAAACCTCTACACGTTCTGCGTGAGCTATGTG
GATTCCATCCAGCAAATGAGGAACAAGTTTGCCTTCCGAGAGGCCATCAACAAACTGGAGAATAATCT
CCGGGAGCTTCAGATCTGCCCGGCGACAGCAGGCAGTGGTCCAGCGGCCACTCAGGACTTCAGCAAGC
TCCTCAGTTCGGTGAAGGAAATCAGTGACATAGTGCAGAGGTAG

In some embodiments, an ABL1 fusion nucleic acid molecule of the disclosure is a BCR-ABL1 fusion nucleic acid molecule, wherein the BCR-ABL1 fusion nucleic acid molecule encodes a BCR-ABL1 fusion polypeptide comprising the amino acid sequence of SEQ ID NO: 34, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 34. In some embodiments, the BCR-ABL1 fusion nucleic acid molecule comprises the nucleotide sequence of SEQ ID NO: 13, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 13.

(SEQ ID NO: 13)
ATGGTGGACCCGGTGGGCTTCGCGGAGGCGTGGAAGGCGCAGTTCCCGGACTCAGAGCCCCCGCGCAT
GGAGCTGCGCTCAGTGGGCGACATCGAGCAGGAGCTGGAGCGCTGCAAGGCCTCCATTCGGCGCCTGG
AGCAGGAGGTGAACCAGGAGCGCTTCCGCATGATCTACCTGCAGACGTTGCTGGCCAAGGAAAAGAAG
AGCTATGACCGGCAGCGATGGGGCTTCCGGCGCGCGGCGCAGGCCCCCGACGGCGCCTCCGAGCCCCG
AGCGTCCGCGTCGCGCCCGCAGCCAGCGCCCGCCGACGGAGCCGACCCGCCGCCCGCCGAGGAGCCCG
AGGCCCGGCCCGACGGCGAGGGTTCTCCGGGTAAGGCCAGGCCCGGGACCGCCCGCAGGCCCGGGGCA
GCCGCGTCGGGGGAACGGGACGACCGGGGACCCCCCGCCAGCGTGGCGGCGCTCAGGTCCAACTTCGA
GCGGATCCGCAAGGGCCATGGCCAGCCCGGGGCGGACGCCGAGAAGCCCTTCTACGTGAACGTCGAGT
TTCACCACGAGCGCGGCCTGGTGAAGGTCAACGACAAAGAGGTGTCGGACCGCATCAGCTCCCTGGGC
AGCCAGGCCATGCAGATGGAGCGCAAAAAGTCCCAGCACGGCGCGGGCTCGAGCGTGGGGGATGCATC
CAGGCCCCCTTACCGGGGACGCTCCTCGGAGAGCAGCTGCGGCGTCGACGGCGACTACGAGGACGCCG
AGTTGAACCCCCGCTTCCTGAAGGACAACCTGATCGACGCCAATGGCGGTAGCAGGCCCCCTTGGCCG
CCCCTGGAGTACCAGCCCTACCAGAGCATCTACGTCGGGGGCATGATGGAAGGGGAGGGCAAGGGCCC
GCTCCTGCGCAGCCAGAGCACCTCTGAGCAGGAGAAGCGCCTTACCTGGCCCCGCAGGTCCTACTCCC
CCCGGAGTTTTGAGGATTGCGGAGGCGGCTATACCCCGGACTGCAGCTCCAATGAGAACCTCACCTCC
AGCGAGGAGGACTTCTCCTCTGGCCAGTCCAGCCGCGTGTCCCCAAGCCCCACCACCTACCGCATGTT
CCGGGACAAAAGCCGCTCTCCCTCGCAGAACTCGCAACAGTCCTTCGACAGCAGCAGTCCCCCCACGC
CGCAGTGCCATAAGCGGCACCGGCACTGCCCGGTTGTCGTGTCCGAGGCCACCATCGTGGGCGTCCGC
AAGACCGGGCAGATCTGGCCCAACGATGGCGAGGGCGCCTTCCATGGAGACGCAGATGGCTCGTTCGG
AACACCACCTGGATACGGCTGCGCTGCAGACCGGGCAGAGGAGCAGCGCCGGCACCAAGATGGGCTGC
CCTACATTGATGACTCGCCCTCCTCATCGCCCCACCTCAGCAGCAAGGGCAGGGGCAGCCGGGATGCG
CTGGTCTCGGGAGCCCTGGAGTCCACTAAAGCGAGTGAGCTGGACTTGGAAAAGGGCTTGGAGATGAG
AAAATGGGTCCTGTCGGGAATCCTGGCTAGCGAGGAGACTTACCTGAGCCACCTGGAGGCACTGCTGC
TGCCCATGAAGCCTTTGAAAGCCGCTGCCACCACCTCTCAGCCGGTGCTGACGAGTCAGCAGATCGAG
ACCATCTTCTTCAAAGTGCCTGAGCTCTACGAGATCCACAAGGAGTTCTATGATGGGCTCTTCCCCCG
CGTGCAGCAGTGGAGCCACCAGCAGCGGGTGGGCGACCTCTTCCAGAAGCTGGCCAGCCAGCTGGGTG
TGTACCGGGCCTTCGTGGACAACTACGGAGTTGCCATGGAAATGGCTGAGAAGTGCTGTCAGGCCAAT
GCTCAGTTTGCAGAAATCTCCGAGAACCTGAGAGCCAGAAGCAACAAAGATGCCAAGGATCCAACGAC
CAAGAACTCTCTGGAAACTCTGCTCTACAAGCCTGTGGACCGTGTGACGAGGAGCACGCTGGTCCTCC
ATGACTTGCTGAAGCACACTCCTGCCAGCCACCCTGACCACCCCTTGCTGCAGGACGCCCTCCGCATC
TCACAGAACTTCCTGTCCAGCATCAATGAGGAGATCACACCCCGACGGCAGTCCATGACGGTGAAGAA
GGGAGAGCACCGGCAGCTGCTGAAGGACAGCTTCATGGTGGAGCTGGTGGAGGGGGCCCGCAAGCTGC
GCCACGTCTTCCTGTTCACCGACCTGCTTCTCTGCACCAAGCTCAAGAAGCAGAGCGGAGGCAAAACG
CAGCAGTATGACTGCAAATGGTACATTCCGCTCACGGATCTCAGCTTCCAGATGGTGGATGAACTGGA
GGCAGTGCCCAACATCCCCCTGGTGCCCGATGAGGAGCTGGACGCTTTGAAGATCAAGATCTCCCAGA
TCAAGAATGACATCCAGAGAGAGAAGAGGGCGAACAAGGGCAGCAAGGCTACGGAGAGGCTGAAGAAG
AAGCTGTCGGAGCAGGAGTCACTGCTGCTGCTTATGTCTCCCAGCATGGCCTTCAGGGTGCACAGCCG
CAACGGCAAGAGTTACACGTTCCTGATCTCCTCTGACTATGAGCGTGCAGAGTGGAGGGAGAACATCC
GGGAGCAGCAGAAGAAGTGTTTCAGAAGCTTCTCCCTGACATCCGTGGAGCTGCAGATGCTGACCAAC
TCGTGTGTGAAACTCCAGACTGTCCACAGCATTCCGCTGACCATCAATAAGGAAGAAGCCCTTCAGCG
GCCAGTAGCATCTGACTTTGAGCCTCAGGGTCTGAGTGAAGCCGCTCGTTGGAACTCCAAGGAAAACC
TTCTCGCTGGACCCAGTGAAAATGACCCCAACCTTTTCGTTGCACTGTATGATTTTGTGGCCAGTGGA
GATAACACTCTAAGCATAACTAAAGGTGAAAAGCTCCGGGTCTTAGGCTATAATCACAATGGGGAATG
GTGTGAAGCCCAAACCAAAAATGGCCAAGGCTGGGTCCCAAGCAACTACATCACGCCAGTCAACAGTC
TGGAGAAACACTCCTGGTACCATGGGCCTGTGTCCCGCAATGCCGCTGAGTATCTGCTGAGCAGCGGG
ATCAATGGCAGCTTCTTGGTGCGTGAGAGTGAGAGCAGTCCTGGCCAGAGGTCCATCTCGCTGAGATA
CGAAGGGAGGGTGTACCATTACAGGATCAACACTGCTTCTGATGGCAAGCTCTACGTCTCCTCCGAGA
GCCGCTTCAACACCCTGGCCGAGTTGGTTCATCATCATTCAACGGTGGCCGACGGGCTCATCACCACG
CTCCATTATCCAGCCCCAAAGCGCAACAAGCCCACTGTCTATGGTGTGTCCCCCAACTACGACAAGTG
GGAGATGGAACGCACGGACATCACCATGAAGCACAAGCTGGGCGGGGGCCAGTACGGGGAGGTGTACG
AGGGCGTGTGGAAGAAATACAGCCTGACGGTGGCCGTGAAGACCTTGAAGGAGGACACCATGGAGGTG
GAAGAGTTCTTGAAAGAAGCTGCAGTCATGAAAGAGATCAAACACCCTAACCTGGTGCAGCTCCTTGG
GGTCTGCACCCGGGAGCCCCCGTTCTATATCATCACTGAGTTCATGACCTACGGGAACCTCCTGGACT
ACCTGAGGGAGTGCAACCGGCAGGAGGTGAACGCCGTGGTGCTGCTGTACATGGCCACTCAGATCTCG
TCAGCCATGGAGTACCTGGAGAAGAAAAACTTCATCCACAGAGATCTTGCTGCCCGAAACTGCCTGGT
AGGGGAGAACCACTTGGTGAAGGTAGCTGATTTTGGCCTGAGCAGGTTGATGACAGGGGACACCTACA
CAGCCCATGCTGGAGCCAAGTTCCCCATCAAATGGACTGCACCCGAGAGCCTGGCCTACAACAAGTTC
TCCATCAAGTCCGACGTCTGGGCATTTGGAGTATTGCTTTGGGAAATTGCTACCTATGGCATGTCCCC
TTACCCGGGAATTGACCTGTCCCAGGTGTATGAGCTGCTAGAGAAGGACTACCGCATGGAGCGCCCAG
AAGGCTGCCCAGAGAAGGTCTATGAACTCATGCGAGCATGTTGGCAGTGGAATCCCTCTGACCGGCCC
TCCTTTGCTGAAATCCACCAAGCCTTTGAAACAATGTTCCAGGAATCCAGTATCTCAGACGAAGTGGA
AAAGGAGCTGGGGAAACAAGGCGTCCGTGGGGCTGTGAGTACCTTGCTGCAGGCCCCAGAGCTGCCCA
CCAAGACGAGGACCTCCAGGAGAGCTGCAGAGCACAGAGACACCACTGACGTGCCTGAGATGCCTCAC
TCCAAGGGCCAGGGAGAGAGCGATCCTCTGGACCATGAGCCTGCCGTGTCTCCATTGCTCCCTCGAAA
AGAGCGAGGTCCCCCGGAGGGCGGCCTGAATGAAGATGAGCGCCTTCTCCCCAAAGACAAAAAGACCA
ACTTGTTCAGCGCCTTGATCAAGAAGAAGAAGAAGACAGCCCCAACCCCTCCCAAACGCAGCAGCTCC
TTCCGGGAGATGGACGGCCAGCCGGAGCGCAGAGGGGCCGGCGAGGAAGAGGGCCGAGACATCAGCAA
CGGGGCACTGGCTTTCACCCCCTTGGACACAGCTGACCCAGCCAAGTCCCCAAAGCCCAGCAATGGGG
CTGGGGTCCCCAATGGAGCCCTCCGGGAGTCCGGGGGCTCAGGCTTCCGGTCTCCCCACCTGTGGAAG
AAGTCCAGCACGCTGACCAGCAGCCGCCTAGCCACCGGCGAGGAGGAGGGCGGTGGCAGCTCCAGCAA
GCGCTTCCTGCGCTCTTGCTCCGCCTCCTGCGTTCCCCATGGGGCCAAGGACACGGAGTGGAGGTCAG
TCACGCTGCCTCGGGACTTGCAGTCCACGGGAAGACAGTTTGACTCGTCCACATTTGGAGGGCACAAA
AGTGAGAAGCCGGCTCTGCCTCGGAAGAGGGCAGGGGAGAACAGGTCTGACCAGGTGACCCGAGGCAC
AGTAACGCCTCCCCCCAGGCTGGTGAAAAAGAATGAGGAAGCTGCTGATGAGGTCTTCAAAGACATCA
TGGAGTCCAGCCCGGGCTCCAGCCCGCCCAACCTGACTCCAAAACCCCTCCGGCGGCAGGTCACCGTG
GCCCCTGCCTCGGGCCTCCCCCACAAGGAAGAAGCTGGAAAGGGCAGTGCCTTAGGGACCCCTGCTGC
AGCTGAGCCAGTGACCCCCACCAGCAAAGCAGGCTCAGGTGCACCAGGGGGCACCAGCAAGGGCCCCG
CCGAGGAGTCCAGAGTGAGGAGGCACAAGCACTCCTCTGAGTCGCCAGGGAGGGACAAGGGGAAATTG
TCCAGGCTCAAACCTGCCCCGCCGCCCCCACCAGCAGCCTCTGCAGGGAAGGCTGGAGGAAAGCCCTC
GCAGAGCCCGAGCCAGGAGGCGGCCGGGGAGGCAGTCCTGGGCGCAAAGACAAAAGCCACGAGTCTGG
TTGATGCTGTGAACAGTGACGCTGCCAAGCCCAGCCAGCCGGGAGAGGGCCTCAAAAAGCCCGTGCTC
CCGGCCACTCCAAAGCCACAGTCCGCCAAGCCGTCGGGGACCCCCATCAGCCCAGCCCCCGTTCCCTC
CACGTTGCCATCAGCATCCTCGGCCCTGGCAGGGGACCAGCCGTCTTCCACCGCCTTCATCCCTCTCA
TATCAACCCGAGTGTCTCTTCGGAAAACCCGCCAGCCTCCAGAGCGGATCGCCAGCGGCGCCATCACC
AAGGGCGTGGTCCTGGACAGCACCGAGGCGCTGTGCCTCGCCATCTCTAGGAACTCCGAGCAGATGGC
CAGCCACAGCGCAGTGCTGGAGGCCGGCAAAAACCTCTACACGTTCTGCGTGAGCTATGTGGATTCCA
TCCAGCAAATGAGGAACAAGTTTGCCTTCCGAGAGGCCATCAACAAACTGGAGAATAATCTCCGGGAG
CTTCAGATCTGCCCGGCGACAGCAGGCAGTGGTCCAGCGGCCACTCAGGACTTCAGCAAGCTCCTCAG
TTCGGTGAAGGAAATCAGTGACATAGTGCAGAGGTAG

In some embodiments, an ABL1 fusion nucleic acid molecule of the disclosure is a BCR-ABL1 fusion nucleic acid molecule, wherein the BCR-ABL1 fusion nucleic acid molecule encodes a BCR-ABL1 fusion polypeptide comprising the amino acid sequence of SEQ ID NO: 35, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 35. In some embodiments, the BCR-ABL1 fusion nucleic acid molecule comprises the nucleotide sequence of SEQ ID NO: 14, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 14.

(SEQ ID NO: 14)
ATGGTGGACCCGGTGGGCTTCGCGGAGGCGTGGAAGGCGCAGTTCCCGGACTCAGAGCCCCCGCGCAT
GGAGCTGCGCTCAGTGGGCGACATCGAGCAGGAGCTGGAGCGCTGCAAGGCCTCCATTCGGCGCCTGG
AGCAGGAGGTGAACCAGGAGCGCTTCCGCATGATCTACCTGCAGACGTTGCTGGCCAAGGAAAAGAAG
AGCTATGACCGGCAGCGATGGGGCTTCCGGCGCGCGGCGCAGGCCCCCGACGGCGCCTCCGAGCCCCG
AGCGTCCGCGTCGCGCCCGCAGCCAGCGCCCGCCGACGGAGCCGACCCGCCGCCCGCCGAGGAGCCCG
AGGCCCGGCCCGACGGCGAGGGTTCTCCGGGTAAGGCCAGGCCCGGGACCGCCCGCAGGCCCGGGGCA
GCCGCGTCGGGGGAACGGGACGACCGGGGACCCCCCGCCAGCGTGGCGGCGCTCAGGTCCAACTTCGA
GCGGATCCGCAAGGGCCATGGCCAGCCCGGGGCGGACGCCGAGAAGCCCTTCTACGTGAACGTCGAGT
TTCACCACGAGCGCGGCCTGGTGAAGGTCAACGACAAAGAGGTGTCGGACCGCATCAGCTCCCTGGGC
AGCCAGGCCATGCAGATGGAGCGCAAAAAGTCCCAGCACGGCGCGGGCTCGAGCGTGGGGGATGCATC
CAGGCCCCCTTACCGGGGACGCTCCTCGGAGAGCAGCTGCGGCGTCGACGGCGACTACGAGGACGCCG
AGTTGAACCCCCGCTTCCTGAAGGACAACCTGATCGACGCCAATGGCGGTAGCAGGCCCCCTTGGCCG
CCCCTGGAGTACCAGCCCTACCAGAGCATCTACGTCGGGGGCATGATGGAAGGGGAGGGCAAGGGCCC
GCTCCTGCGCAGCCAGAGCACCTCTGAGCAGGAGAAGCGCCTTACCTGGCCCCGCAGGTCCTACTCCC
CCCGGAGTTTTGAGGATTGCGGAGGCGGCTATACCCCGGACTGCAGCTCCAATGAGAACCTCACCTCC
AGCGAGGAGGACTTCTCCTCTGGCCAGTCCAGCCGCGTGTCCCCAAGCCCCACCACCTACCGCATGTT
CCGGGACAAAAGCCGCTCTCCCTCGCAGAACTCGCAACAGTCCTTCGACAGCAGCAGTCCCCCCACGC
CGCAGTGCCATAAGCGGCACCGGCACTGCCCGGTTGTCGTGTCCGAGGCCACCATCGTGGGCGTCCGC
AAGACCGGGCAGATCTGGCCCAACGATGGCGAGGGCGCCTTCCATGGAGACGCAGATGGCTCGTTCGG
AACACCACCTGGATACGGCTGCGCTGCAGACCGGGCAGAGGAGCAGCGCCGGCACCAAGATGGGCTGC
CCTACATTGATGACTCGCCCTCCTCATCGCCCCACCTCAGCAGCAAGGGCAGGGGCAGCCGGGATGCG
CTGGTCTCGGGAGCCCTGGAGTCCACTAAAGCGAGTGAGCTGGACTTGGAAAAGGGCTTGGAGATGAG
AAAATGGGTCCTGTCGGGAATCCTGGCTAGCGAGGAGACTTACCTGAGCCACCTGGAGGCACTGCTGC
TGCCCATGAAGCCTTTGAAAGCCGCTGCCACCACCTCTCAGCCGGTGCTGACGAGTCAGCAGATCGAG
ACCATCTTCTTCAAAGTGCCTGAGCTCTACGAGATCCACAAGGAGTTCTATGATGGGCTCTTCCCCCG
CGTGCAGCAGTGGAGCCACCAGCAGCGGGTGGGCGACCTCTTCCAGAAGCTGGCCAGCCAGCTGGGTG
TGTACCGGGCCTTCGTGGACAACTACGGAGTTGCCATGGAAATGGCTGAGAAGTGCTGTCAGGCCAAT
GCTCAGTTTGCAGAAATCTCCGAGAACCTGAGAGCCAGAAGCAACAAAGATGCCAAGGATCCAACGAC
CAAGAACTCTCTGGAAACTCTGCTCTACAAGCCTGTGGACCGTGTGACGAGGAGCACGCTGGTCCTCC
ATGACTTGCTGAAGCACACTCCTGCCAGCCACCCTGACCACCCCTTGCTGCAGGACGCCCTCCGCATC
TCACAGAACTTCCTGTCCAGCATCAATGAGGAGATCACACCCCGACGGCAGTCCATGACGGTGAAGAA
GGGAGAGCACCGGCAGCTGCTGAAGGACAGCTTCATGGTGGAGCTGGTGGAGGGGGCCCGCAAGCTGC
GCCACGTCTTCCTGTTCACCGACCTGCTTCTCTGCACCAAGCTCAAGAAGCAGAGCGGAGGCAAAACG
CAGCAGTATGACTGCAAATGGTACATTCCGCTCACGGATCTCAGCTTCCAGATGGTGGATGAACTGGA
GGCAGTGCCCAACATCCCCCTGGTGCCCGATGAGGAGCTGGACGCTTTGAAGATCAAGATCTCCCAGA
TCAAGAATGACATCCAGAGAGAGAAGAGGGCGAACAAGGGCAGCAAGGCTACGGAGAGGCTGAAGAAG
AAGCTGTCGGAGCAGGAGTCACTGCTGCTGCTTATGTCTCCCAGCATGGCCTTCAGGGTGCACAGCCG
CAACGGCAAGAGTTACACGTTCCTGATCTCCTCTGACTATGAGCGTGCAGAGTGGAGGGAGAACATCC
GGGAGCAGCAGAAGAAGTGTTTCAGAAGCTTCTCCCTGACATCCGTGGAGCTGCAGATGCTGACCAAC
TCGTGTGTGAAACTCCAGACTGTCCACAGCATTCCGCTGACCATCAATAAGGAAGATGATGAGTCTCC
GGGGCTCTATGGGTTTCTGAATGTCATCGTCCACTCAGCCACTGGATTTAAGCAGAGTTCAAAAGCCC
TTCAGCGGCCAGTAGCATCTGACTTTGAGCCTCAGGGTCTGAGTGAAGCCGCTCGTTGGAACTCCAAG
GAAAACCTTCTCGCTGGACCCAGTGAAAATGACCCCAACCTTTTCGTTGCACTGTATGATTTTGTGGC
CAGTGGAGATAACACTCTAAGCATAACTAAAGGTGAAAAGCTCCGGGTCTTAGGCTATAATCACAATG
GGGAATGGTGTGAAGCCCAAACCAAAAATGGCCAAGGCTGGGTCCCAAGCAACTACATCACGCCAGTC
AACAGTCTGGAGAAACACTCCTGGTACCATGGGCCTGTGTCCCGCAATGCCGCTGAGTATCTGCTGAG
CAGCGGGATCAATGGCAGCTTCTTGGTGCGTGAGAGTGAGAGCAGTCCTGGCCAGAGGTCCATCTCGC
TGAGATACGAAGGGAGGGTGTACCATTACAGGATCAACACTGCTTCTGATGGCAAGCTCTACGTCTCC
TCCGAGAGCCGCTTCAACACCCTGGCCGAGTTGGTTCATCATCATTCAACGGTGGCCGACGGGCTCAT
CACCACGCTCCATTATCCAGCCCCAAAGCGCAACAAGCCCACTGTCTATGGTGTGTCCCCCAACTACG
ACAAGTGGGAGATGGAACGCACGGACATCACCATGAAGCACAAGCTGGGCGGGGGCCAGTACGGGGAG
GTGTACGAGGGCGTGTGGAAGAAATACAGCCTGACGGTGGCCGTGAAGACCTTGAAGGAGGACACCAT
GGAGGTGGAAGAGTTCTTGAAAGAAGCTGCAGTCATGAAAGAGATCAAACACCCTAACCTGGTGCAGC
TCCTTGGGGTCTGCACCCGGGAGCCCCCGTTCTATATCATCACTGAGTTCATGACCTACGGGAACCTC
CTGGACTACCTGAGGGAGTGCAACCGGCAGGAGGTGAACGCCGTGGTGCTGCTGTACATGGCCACTCA
GATCTCGTCAGCCATGGAGTACCTGGAGAAGAAAAACTTCATCCACAGAGATCTTGCTGCCCGAAACT
GCCTGGTAGGGGAGAACCACTTGGTGAAGGTAGCTGATTTTGGCCTGAGCAGGTTGATGACAGGGGAC
ACCTACACAGCCCATGCTGGAGCCAAGTTCCCCATCAAATGGACTGCACCCGAGAGCCTGGCCTACAA
CAAGTTCTCCATCAAGTCCGACGTCTGGGCATTTGGAGTATTGCTTTGGGAAATTGCTACCTATGGCA
TGTCCCCTTACCCGGGAATTGACCTGTCCCAGGTGTATGAGCTGCTAGAGAAGGACTACCGCATGGAG
CGCCCAGAAGGCTGCCCAGAGAAGGTCTATGAACTCATGCGAGCATGTTGGCAGTGGAATCCCTCTGA
CCGGCCCTCCTTTGCTGAAATCCACCAAGCCTTTGAAACAATGTTCCAGGAATCCAGTATCTCAGACG
AAGTGGAAAAGGAGCTGGGGAAACAAGGCGTCCGTGGGGCTGTGAGTACCTTGCTGCAGGCCCCAGAG
CTGCCCACCAAGACGAGGACCTCCAGGAGAGCTGCAGAGCACAGAGACACCACTGACGTGCCTGAGAT
GCCTCACTCCAAGGGCCAGGGAGAGAGCGATCCTCTGGACCATGAGCCTGCCGTGTCTCCATTGCTCC
CTCGAAAAGAGCGAGGTCCCCCGGAGGGCGGCCTGAATGAAGATGAGCGCCTTCTCCCCAAAGACAAA
AAGACCAACTTGTTCAGCGCCTTGATCAAGAAGAAGAAGAAGACAGCCCCAACCCCTCCCAAACGCAG
CAGCTCCTTCCGGGAGATGGACGGCCAGCCGGAGCGCAGAGGGGCCGGCGAGGAAGAGGGCCGAGACA
TCAGCAACGGGGCACTGGCTTTCACCCCCTTGGACACAGCTGACCCAGCCAAGTCCCCAAAGCCCAGC
AATGGGGCTGGGGTCCCCAATGGAGCCCTCCGGGAGTCCGGGGGCTCAGGCTTCCGGTCTCCCCACCT
GTGGAAGAAGTCCAGCACGCTGACCAGCAGCCGCCTAGCCACCGGCGAGGAGGAGGGCGGTGGCAGCT
CCAGCAAGCGCTTCCTGCGCTCTTGCTCCGCCTCCTGCGTTCCCCATGGGGCCAAGGACACGGAGTGG
AGGTCAGTCACGCTGCCTCGGGACTTGCAGTCCACGGGAAGACAGTTTGACTCGTCCACATTTGGAGG
GCACAAAAGTGAGAAGCCGGCTCTGCCTCGGAAGAGGGCAGGGGAGAACAGGTCTGACCAGGTGACCC
GAGGCACAGTAACGCCTCCCCCCAGGCTGGTGAAAAAGAATGAGGAAGCTGCTGATGAGGTCTTCAAA
GACATCATGGAGTCCAGCCCGGGCTCCAGCCCGCCCAACCTGACTCCAAAACCCCTCCGGCGGCAGGT
CACCGTGGCCCCTGCCTCGGGCCTCCCCCACAAGGAAGAAGCTGGAAAGGGCAGTGCCTTAGGGACCC
CTGCTGCAGCTGAGCCAGTGACCCCCACCAGCAAAGCAGGCTCAGGTGCACCAGGGGGCACCAGCAAG
GGCCCCGCCGAGGAGTCCAGAGTGAGGAGGCACAAGCACTCCTCTGAGTCGCCAGGGAGGGACAAGGG
GAAATTGTCCAGGCTCAAACCTGCCCCGCCGCCCCCACCAGCAGCCTCTGCAGGGAAGGCTGGAGGAA
AGCCCTCGCAGAGCCCGAGCCAGGAGGCGGCCGGGGAGGCAGTCCTGGGCGCAAAGACAAAAGCCACG
AGTCTGGTTGATGCTGTGAACAGTGACGCTGCCAAGCCCAGCCAGCCGGGAGAGGGCCTCAAAAAGCC
CGTGCTCCCGGCCACTCCAAAGCCACAGTCCGCCAAGCCGTCGGGGACCCCCATCAGCCCAGCCCCCG
TTCCCTCCACGTTGCCATCAGCATCCTCGGCCCTGGCAGGGGACCAGCCGTCTTCCACCGCCTTCATC
CCTCTCATATCAACCCGAGTGTCTCTTCGGAAAACCCGCCAGCCTCCAGAGCGGATCGCCAGCGGCGC
CATCACCAAGGGCGTGGTCCTGGACAGCACCGAGGCGCTGTGCCTCGCCATCTCTAGGAACTCCGAGC
AGATGGCCAGCCACAGCGCAGTGCTGGAGGCCGGCAAAAACCTCTACACGTTCTGCGTGAGCTATGTG
GATTCCATCCAGCAAATGAGGAACAAGTTTGCCTTCCGAGAGGCCATCAACAAACTGGAGAATAATCT
CCGGGAGCTTCAGATCTGCCCGGCGACAGCAGGCAGTGGTCCAGCGGCCACTCAGGACTTCAGCAAGC
TCCTCAGTTCGGTGAAGGAAATCAGTGACATAGTGCAGAGGTAG

In some embodiments, an ABL1 fusion nucleic acid molecule of the disclosure is a BCR-ABL1 fusion nucleic acid molecule, wherein the BCR-ABL1 fusion nucleic acid molecule encodes a BCR-ABL1 fusion polypeptide comprising the amino acid sequence of SEQ ID NO: 36, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 36. In some embodiments, the BCR-ABL1 fusion nucleic acid molecule comprises the nucleotide sequence of SEQ ID NO: 15, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 15.

(SEQ ID NO: 15)
ATGGTGGACCCGGTGGGCTTCGCGGAGGCGTGGAAGGCGCAGTTCCCGGACTCAGAGCCCCCGCGCAT
GGAGCTGCGCTCAGTGGGCGACATCGAGCAGGAGCTGGAGCGCTGCAAGGCCTCCATTCGGCGCCTGG
AGCAGGAGGTGAACCAGGAGCGCTTCCGCATGATCTACCTGCAGACGTTGCTGGCCAAGGAAAAGAAG
AGCTATGACCGGCAGCGATGGGGCTTCCGGCGCGCGGCGCAGGCCCCCGACGGCGCCTCCGAGCCCCG
AGCGTCCGCGTCGCGCCCGCAGCCAGCGCCCGCCGACGGAGCCGACCCGCCGCCCGCCGAGGAGCCCG
AGGCCCGGCCCGACGGCGAGGGTTCTCCGGGTAAGGCCAGGCCCGGGACCGCCCGCAGGCCCGGGGCA
GCCGCGTCGGGGGAACGGGACGACCGGGGACCCCCCGCCAGCGTGGCGGCGCTCAGGTCCAACTTCGA
GCGGATCCGCAAGGGCCATGGCCAGCCCGGGGCGGACGCCGAGAAGCCCTTCTACGTGAACGTCGAGT
TTCACCACGAGCGCGGCCTGGTGAAGGTCAACGACAAAGAGGTGTCGGACCGCATCAGCTCCCTGGGC
AGCCAGGCCATGCAGATGGAGCGCAAAAAGTCCCAGCACGGCGCGGGCTCGAGCGTGGGGGATGCATC
CAGGCCCCCTTACCGGGGACGCTCCTCGGAGAGCAGCTGCGGCGTCGACGGCGACTACGAGGACGCCG
AGTTGAACCCCCGCTTCCTGAAGGACAACCTGATCGACGCCAATGGCGGTAGCAGGCCCCCTTGGCCG
CCCCTGGAGTACCAGCCCTACCAGAGCATCTACGTCGGGGGCATGATGGAAGGGGAGGGCAAGGGCCC
GCTCCTGCGCAGCCAGAGCACCTCTGAGCAGGAGAAGCGCCTTACCTGGCCCCGCAGGTCCTACTCCC
CCCGGAGTTTTGAGGATTGCGGAGGCGGCTATACCCCGGACTGCAGCTCCAATGAGAACCTCACCTCC
AGCGAGGAGGACTTCTCCTCTGGCCAGTCCAGCCGCGTGTCCCCAAGCCCCACCACCTACCGCATGTT
CCGGGACAAAAGCCGCTCTCCCTCGCAGAACTCGCAACAGTCCTTCGACAGCAGCAGTCCCCCCACGC
CGCAGTGCCATAAGCGGCACCGGCACTGCCCGGTTGTCGTGTCCGAGGCCACCATCGTGGGCGTCCGC
AAGACCGGGCAGATCTGGCCCAACGATGGCGAGGGCGCCTTCCATGGAGACGCAGATGGCTCGTTCGG
AACACCACCTGGATACGGCTGCGCTGCAGACCGGGCAGAGGAGCAGCGCCGGCACCAAGATGGGCTGC
CCTACATTGATGACTCGCCCTCCTCATCGCCCCACCTCAGCAGCAAGGGCAGGGGCAGCCGGGATGCG
CTGGTCTCGGGAGCCCTGGAGTCCACTAAAGCGAGTGAGCTGGACTTGGAAAAGGGCTTGGAGATGAG
AAAATGGGTCCTGTCGGGAATCCTGGCTAGCGAGGAGACTTACCTGAGCCACCTGGAGGCACTGCTGC
TGCCCATGAAGCCTTTGAAAGCCGCTGCCACCACCTCTCAGCCGGTGCTGACGAGTCAGCAGATCGAG
ACCATCTTCTTCAAAGTGCCTGAGCTCTACGAGATCCACAAGGAGTTCTATGATGGGCTCTTCCCCCG
CGTGCAGCAGTGGAGCCACCAGCAGCGGGTGGGCGACCTCTTCCAGAAGCTGGCCAGCCAGCTGGGTG
TGTACCGGGCCTTCGTGGACAACTACGGAGTTGCCATGGAAATGGCTGAGAAGTGCTGTCAGGCCAAT
GCTCAGTTTGCAGAAATCTCCGAGAACCTGAGAGCCAGAAGCAACAAAGATGCCAAGGATCCAACGAC
CAAGAACTCTCTGGAAACTCTGCTCTACAAGCCTGTGGACCGTGTGACGAGGAGCACGCTGGTCCTCC
ATGACTTGCTGAAGCACACTCCTGCCAGCCACCCTGACCACCCCTTGCTGCAGGACGCCCTCCGCATC
TCACAGAACTTCCTGTCCAGCATCAATGAGGAGATCACACCCCGACGGCAGTCCATGACGGTGAAGAA
GGGAGAGCACCGGCAGCTGCTGAAGGACAGCTTCATGGTGGAGCTGGTGGAGGGGGCCCGCAAGCTGC
GCCACGTCTTCCTGTTCACCGACCTGCTTCTCTGCACCAAGCTCAAGAAGCAGAGCGGAGGCAAAACG
CAGCAGTATGACTGCAAATGGTACATTCCGCTCACGGATCTCAGCTTCCAGATGGTGGATGAACTGGA
GGCAGTGCCCAACATCCCCCTGGTGCCCGATGAGGAGCTGGACGCTTTGAAGATCAAGATCTCCCAGA
TCAAGAATGACATCCAGAGAGAGAAGAGGGCGAACAAGGGCAGCAAGGCTACGGAGAGGCTGAAGAAG
AAGCTGTCGGAGCAGGAGTCACTGCTGCTGCTTATGTCTCCCAGCATGGCCTTCAGGGTGCACAGCCG
CAACGGCAAGAGTTACACGTTCCTGATCTCCTCTGACTATGAGCGTGCAGAGTGGAGGGAGAACATCC
GGGAGCAGCAGAAGAAGTGTTTCAGAAGCTTCTCCCTGACATCCGTGGAGCTGCAGATGCTGACCAAC
TCGTGTGTGAAACTCCAGACTGTCCACAGCATTCCGCTGACCATCAATAAGGAAGATGATGAGTCTCC
GGGGCTCTATGGGTTTCTGAATGTCATCGTCCACTCAGCCACTGGATTTAAGCAGAGTTCAAAAGCCC
TTCAGCGGCCAGTAGCATCTGACTTTGAGCCTCAGGGTCTGAGTGAAGCCGCTCGTTGGAACTCCAAG
GAAAACCTTCTCGCTGGACCCAGTGAAAATGACCCCAACCTTTTCGTTGCACTGTATGATTTTGTGGC
CAGTGGAGATAACACTCTAAGCATAACTAAAGGTGAAAAGCTCCGGGTCTTAGGCTATAATCACAATG
GGGAATGGTGTGAAGCCCAAACCAAAAATGGCCAAGGCTGGGTCCCAAGCAACTACATCACGCCAGTC
AACAGTCTGGAGAAACACTCCTGGTACCATGGGCCTGTGTCCCGCAATGCCGCTGAGTATCTGCTGAG
CAGCGGGATCAATGGCAGCTTCTTGGTGCGTGAGAGTGAGAGCAGTCCTGGCCAGAGGTCCATCTCGC
TGAGATACGAAGGGAGGGTGTACCATTACAGGATCAACACTGCTTCTGATGGCAAGCTCTACGTCTCC
TCCGAGAGCCGCTTCAACACCCTGGCCGAGTTGGTTCATCATCATTCAACGGTGGCCGACGGGCTCAT
CACCACGCTCCATTATCCAGCCCCAAAGCGCAACAAGCCCACTGTCTATGGTGTGTCCCCCAACTACG
ACAAGTGGGAGATGGAACGCACGGACATCACCATGAAGCACAAGCTGGGCGGGGGCCAGTACGGGGAG
GTGTACGAGGGCGTGTGGAAGAAATACAGCCTGACGGTGGCCGTGAAGACCTTGAAGGAGGACACCAT
GGAGGTGGAAGAGTTCTTGAAAGAAGCTGCAGTCATGAAAGAGATCAAACACCCTAACCTGGTGCAGC
TCCTTGGGGTCTGCACCCGGGAGCCCCCGTTCTATATCATCACTGAGTTCATGACCTACGGGAACCTC
CTGGACTACCTGAGGGAGTGCAACCGGCAGGAGGTGAACGCCGTGGTGCTGCTGTACATGGCCACTCA
GATCTCGTCAGCCATGGAGTACCTGGAGAAGAAAAACTTCATCCACAGAGATCTTGCTGCCCGAAACT
GCCTGGTAGGGGAGAACCACTTGGTGAAGGTAGCTGATTTTGGCCTGAGCAGGTTGATGACAGGGGAC
ACCTACACAGCCCATGCTGGAGCCAAGTTCCCCATCAAATGGACTGCACCCGAGAGCCTGGCCTACAA
CAAGTTCTCCATCAAGTCCGACGTCTGGGCATTTGGAGTATTGCTTTGGGAAATTGCTACCTATGGCA
TGTCCCCTTACCCGGGAATTGACCTGTCCCAGGTGTATGAGCTGCTAGAGAAGGACTACCGCATGGAG
CGCCCAGAAGGCTGCCCAGAGAAGGTCTATGAACTCATGCGAGCATGTTGGCAGTGGAATCCCTCTGA
CCGGCCCTCCTTTGCTGAAATCCACCAAGCCTTTGAAACAATGTTCCAGGAATCCAGTATCTCAGACG
AAGTGGAAAAGGAGCTGGGGAAACAAGGCGTCCGTGGGGCTGTGAGTACCTTGCTGCAGGCCCCAGAG
CTGCCCACCAAGACGAGGACCTCCAGGAGAGCTGCAGAGCACAGAGACACCACTGACGTGCCTGAGAT
GCCTCACTCCAAGGGCCAGGGAGAGAGCGATCCTCTGGACCATGAGCCTGCCGTGTCTCCATTGCTCC
CTCGAAAAGAGCGAGGTCCCCCGGAGGGCGGCCTGAATGAAGATGAGCGCCTTCTCCCCAAAGACAAA
AAGACCAACTTGTTCAGCGCCTTGATCAAGAAGAAGAAGAAGACAGCCCCAACCCCTCCCAAACGCAG
CAGCTCCTTCCGGGAGATGGACGGCCAGCCGGAGCGCAGAGGGGCCGGCGAGGAAGAGGGCCGAGACA
TCAGCAACGGGGCACTGGCTTTCACCCCCTTGGACACAGCTGACCCAGCCAAGTCCCCAAAGCCCAGC
AATGGGGCTGGGGTCCCCAATGGAGCCCTCCGGGAGTCCGGGGGCTCAGGCTTCCGGTCTCCCCACCT
GTGGAAGAAGTCCAGCACGCTGACCAGCAGCCGCCTAGCCACCGGCGAGGAGGAGGGCGGTGGCAGCT
CCAGCAAGCGCTTCCTGCGCTCTTGCTCCGCCTCCTGCGTTCCCCATGGGGCCAAGGACACGGAGTGG
AGGTCAGTCACGCTGCCTCGGGACTTGCAGTCCACGGGAAGACAGTTTGACTCGTCCACATTTGGAGG
GCACAAAAGTGAGAAGCCGGCTCTGCCTCGGAAGAGGGCAGGGGAGAACAGGTCTGACCAGGTGACCC
GAGGCACAGTAACGCCTCCCCCCAGGCTGGTGAAAAAGAATGAGGAAGCTGCTGATGAGGTCTTCAAA
GACATCATGGAGTCCAGCCCGGGCTCCAGCCCGCCCAACCTGACTCCAAAACCCCTCCGGCGGCAGGT
CACCGTGGCCCCTGCCTCGGGCCTCCCCCACAAGGAAGAAGCTGGAAAGGGCAGTGCCTTAGGGACCC
CTGCTGCAGCTGAGCCAGTGACCCCCACCAGCAAAGCAGGCTCAGGTGCACCAGGGGGCACCAGCAAG
GGCCCCGCCGAGGAGTCCAGAGTGAGGAGGCACAAGCACTCCTCTGAGTCGCCAGGGAGGGACAAGGG
GAAATTGTCCAGGCTCAAACCTGCCCCGCCGCCCCCACCAGCAGCCTCTGCAGGGAAGGCTGGAGGAA
AGCCCTCGCAGAGCCCGAGCCAGGAGGCGGCCGGGGAGGCAGTCCTGGGCGCAAAGACAAAAGCCACG
AGTCTGGTTGATGCTGTGAACAGTGACGCTGCCAAGCCCAGCCAGCCGGGAGAGGGCCTCAAAAAGCC
CGTGCTCCCGGCCACTCCAAAGCCACAGTCCGCCAAGCCGTCGGGGACCCCCATCAGCCCAGCCCCCG
TTCCCTCCACGTTGCCATCAGCATCCTCGGCCCTGGCAGGGGACCAGCCGTCTTCCACCGCCTTCATC
CCTCTCATATCAACCCGAGTGTCTCTTCGGAAAACCCGCCAGCCTCCAGAGCGGATCGCCAGCGGCGC
CATCACCAAGGGCGTGGTCCTGGACAGCACCGAGGCGCTGTGCCTCGCCATCTCTAGGAACTCCGAGC
AGATGGCCAGCCACAGCGCAGTGCTGGAGGCCGGCAAAAACCTCTACACGTTCTGCGTGAGCTATGTG
GATTCCATCCAGCAAATGAGGAACAAGTTTGCCTTCCGAGAGGCCATCAACAAACTGGAGAATAATCT
CCGGGAGCTTCAGATCTGCCCGGCGACAGCAGGCAGTGGTCCAGCGGCCACTCAGGACTTCAGCAAGC
TCCTCAGTTCGGTGAAGGAAATCAGTGACATAGTGCAGAGGTAG

In some embodiments, an ABL1 fusion nucleic acid molecule of the disclosure is a BCR-ABL1 fusion nucleic acid molecule, wherein the BCR-ABL1 fusion nucleic acid molecule encodes a BCR-ABL1 fusion polypeptide comprising the amino acid sequence of SEQ ID NO: 37, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 37. In some embodiments, the BCR-ABL1 fusion nucleic acid molecule comprises the nucleotide sequence of SEQ ID NO: 16, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 16.

(SEQ ID NO: 16)
ATGGTGGACCCGGTGGGCTTCGCGGAGGCGTGGAAGGCGCAGTTCCCGGACTCAGAGCCCCCGCGCAT
GGAGCTGCGCTCAGTGGGCGACATCGAGCAGGAGCTGGAGCGCTGCAAGGCCTCCATTCGGCGCCTGG
AGCAGGAGGTGAACCAGGAGCGCTTCCGCATGATCTACCTGCAGACGTTGCTGGCCAAGGAAAAGAAG
AGCTATGACCGGCAGCGATGGGGCTTCCGGCGCGCGGCGCAGGCCCCCGACGGCGCCTCCGAGCCCCG
AGCGTCCGCGTCGCGCCCGCAGCCAGCGCCCGCCGACGGAGCCGACCCGCCGCCCGCCGAGGAGCCCG
AGGCCCGGCCCGACGGCGAGGGTTCTCCGGGTAAGGCCAGGCCCGGGACCGCCCGCAGGCCCGGGGCA
GCCGCGTCGGGGGAACGGGACGACCGGGGACCCCCCGCCAGCGTGGCGGCGCTCAGGTCCAACTTCGA
GCGGATCCGCAAGGGCCATGGCCAGCCCGGGGCGGACGCCGAGAAGCCCTTCTACGTGAACGTCGAGT
TTCACCACGAGCGCGGCCTGGTGAAGGTCAACGACAAAGAGGTGTCGGACCGCATCAGCTCCCTGGGC
AGCCAGGCCATGCAGATGGAGCGCAAAAAGTCCCAGCACGGCGCGGGCTCGAGCGTGGGGGATGCATC
CAGGCCCCCTTACCGGGGACGCTCCTCGGAGAGCAGCTGCGGCGTCGACGGCGACTACGAGGACGCCG
AGTTGAACCCCCGCTTCCTGAAGGACAACCTGATCGACGCCAATGGCGGTAGCAGGCCCCCTTGGCCG
CCCCTGGAGTACCAGCCCTACCAGAGCATCTACGTCGGGGGCATGATGGAAGGGGAGGGCAAGGGCCC
GCTCCTGCGCAGCCAGAGCACCTCTGAGCAGGAGAAGCGCCTTACCTGGCCCCGCAGGTCCTACTCCC
CCCGGAGTTTTGAGGATTGCGGAGGCGGCTATACCCCGGACTGCAGCTCCAATGAGAACCTCACCTCC
AGCGAGGAGGACTTCTCCTCTGGCCAGTCCAGCCGCGTGTCCCCAAGCCCCACCACCTACCGCATGTT
CCGGGACAAAAGCCGCTCTCCCTCGCAGAACTCGCAACAGTCCTTCGACAGCAGCAGTCCCCCCACGC
CGCAGTGCCATAAGCGGCACCGGCACTGCCCGGTTGTCGTGTCCGAGGCCACCATCGTGGGCGTCCGC
AAGACCGGGCAGATCTGGCCCAACGATGGCGAGGGCGCCTTCCATGGAGACGCAGATGGCTCGTTCGG
AACACCACCTGGATACGGCTGCGCTGCAGACCGGGCAGAGGAGCAGCGCCGGCACCAAGATGGGCTGC
CCTACATTGATGACTCGCCCTCCTCATCGCCCCACCTCAGCAGCAAGGGCAGGGGCAGCCGGGATGCG
CTGGTCTCGGGAGCCCTGGAGTCCACTAAAGCGAGTGAGCTGGACTTGGAAAAGGGCTTGGAGATGAG
AAAATGGGTCCTGTCGGGAATCCTGGCTAGCGAGGAGACTTACCTGAGCCACCTGGAGGCACTGCTGC
TGCCCATGAAGCCTTTGAAAGCCGCTGCCACCACCTCTCAGCCGGTGCTGACGAGTCAGCAGATCGAG
ACCATCTTCTTCAAAGTGCCTGAGCTCTACGAGATCCACAAGGAGTTCTATGATGGGCTCTTCCCCCG
CGTGCAGCAGTGGAGCCACCAGCAGCGGGTGGGCGACCTCTTCCAGAAGCTGGCCAGCCAGCTGGGTG
TGTACCGGGCCTTCGTGGACAACTACGGAGTTGCCATGGAAATGGCTGAGAAGTGCTGTCAGGCCAAT
GCTCAGTTTGCAGAAATCTCCGAGAACCTGAGAGCCAGAAGCAACAAAGATGCCAAGGATCCAACGAC
CAAGAACTCTCTGGAAACTCTGCTCTACAAGCCTGTGGACCGTGTGACGAGGAGCACGCTGGTCCTCC
ATGACTTGCTGAAGCACACTCCTGCCAGCCACCCTGACCACCCCTTGCTGCAGGACGCCCTCCGCATC
TCACAGAACTTCCTGTCCAGCATCAATGAGGAGATCACACCCCGACGGCAGTCCATGACGGTGAAGAA
GGGAGAGCACCGGCAGCTGCTGAAGGACAGCTTCATGGTGGAGCTGGTGGAGGGGGCCCGCAAGCTGC
GCCACGTCTTCCTGTTCACCGACCTGCTTCTCTGCACCAAGCTCAAGAAGCAGAGCGGAGGCAAAACG
CAGCAGTATGACTGCAAATGGTACATTCCGCTCACGGATCTCAGCTTCCAGATGGTGGATGAACTGGA
GGCAGTGCCCAACATCCCCCTGGTGCCCGATGAGGAGCTGGACGCTTTGAAGATCAAGATCTCCCAGA
TCAAGAATGACATCCAGAGAGAGAAGAGGGCGAACAAGGGCAGCAAGGCTACGGAGAGGCTGAAGAAG
AAGCTGTCGGAGCAGGAGTCACTGCTGCTGCTTATGTCTCCCAGCATGGCCTTCAGGGTGCACAGCCG
CAACGGCAAGAGTTACACGTTCCTGATCTCCTCTGACTATGAGCGTGCAGAGTGGAGGGAGAACATCC
GGGAGCAGCAGAAGAAGTGTTTCAGAAGCTTCTCCCTGACATCCGTGGAGCTGCAGATGCTGACCAAC
TCGTGTGTGAAACTCCAGACTGTCCACAGCATTCCGCTGACCATCAATAAGGAAGATGATGAGTCTCC
GGGGCTCTATGGGTTTCTGAATGTCATCGTCCACTCAGCCACTGGATTTAAGCAGAGTTCAAAAGCCC
TTCAGCGGCCAGTAGCATCTGACTTTGAGCCTCAGGGTCTGAGTGAAGCCGCTCGTTGGAACTCCAAG
GAAAACCTTCTCGCTGGACCCAGTGAAAATGACCCCAACCTTTTCGTTGCACTGTATGATTTTGTGGC
CAGTGGAGATAACACTCTAAGCATAACTAAAGGTGAAAAGCTCCGGGTCTTAGGCTATAATCACAATG
GGGAATGGTGTGAAGCCCAAACCAAAAATGGCCAAGGCTGGGTCCCAAGCAACTACATCACGCCAGTC
AACAGTCTGGAGAAACACTCCTGGTACCATGGGCCTGTGTCCCGCAATGCCGCTGAGTATCTGCTGAG
CAGCGGGATCAATGGCAGCTTCTTGGTGCGTGAGAGTGAGAGCAGTCCTGGCCAGAGGTCCATCTCGC
TGAGATACGAAGGGAGGGTGTACCATTACAGGATCAACACTGCTTCTGATGGCAAGCTCTACGTCTCC
TCCGAGAGCCGCTTCAACACCCTGGCCGAGTTGGTTCATCATCATTCAACGGTGGCCGACGGGCTCAT
CACCACGCTCCATTATCCAGCCCCAAAGCGCAACAAGCCCACTGTCTATGGTGTGTCCCCCAACTACG
ACAAGTGGGAGATGGAACGCACGGACATCACCATGAAGCACAAGCTGGGCGGGGGCCAGTACGGGGAG
GTGTACGAGGGCGTGTGGAAGAAATACAGCCTGACGGTGGCCGTGAAGACCTTGAAGGAGGACACCAT
GGAGGTGGAAGAGTTCTTGAAAGAAGCTGCAGTCATGAAAGAGATCAAACACCCTAACCTGGTGCAGC
TCCTTGGGGTCTGCACCCGGGAGCCCCCGTTCTATATCATCACTGAGTTCATGACCTACGGGAACCTC
CTGGACTACCTGAGGGAGTGCAACCGGCAGGAGGTGAACGCCGTGGTGCTGCTGTACATGGCCACTCA
GATCTCGTCAGCCATGGAGTACCTGGAGAAGAAAAACTTCATCCACAGAGATCTTGCTGCCCGAAACT
GCCTGGTAGGGGAGAACCACTTGGTGAAGGTAGCTGATTTTGGCCTGAGCAGGTTGATGACAGGGGAC
ACCTACACAGCCCATGCTGGAGCCAAGTTCCCCATCAAATGGACTGCACCCGAGAGCCTGGCCTACAA
CAAGTTCTCCATCAAGTCCGACGTCTGGGCATTTGGAGTATTGCTTTGGGAAATTGCTACCTATGGCA
TGTCCCCTTACCCGGGAATTGACCTGTCCCAGGTGTATGAGCTGCTAGAGAAGGACTACCGCATGGAG
CGCCCAGAAGGCTGCCCAGAGAAGGTCTATGAACTCATGCGAGCATGTTGGCAGTGGAATCCCTCTGA
CCGGCCCTCCTTTGCTGAAATCCACCAAGCCTTTGAAACAATGTTCCAGGAATCCAGTATCTCAGACG
AAGTGGAAAAGGAGCTGGGGAAACAAGGCGTCCGTGGGGCTGTGAGTACCTTGCTGCAGGCCCCAGAG
CTGCCCACCAAGACGAGGACCTCCAGGAGAGCTGCAGAGCACAGAGACACCACTGACGTGCCTGAGAT
GCCTCACTCCAAGGGCCAGGGAGAGAGCGATCCTCTGGACCATGAGCCTGCCGTGTCTCCATTGCTCC
CTCGAAAAGAGCGAGGTCCCCCGGAGGGCGGCCTGAATGAAGATGAGCGCCTTCTCCCCAAAGACAAA
AAGACCAACTTGTTCAGCGCCTTGATCAAGAAGAAGAAGAAGACAGCCCCAACCCCTCCCAAACGCAG
CAGCTCCTTCCGGGAGATGGACGGCCAGCCGGAGCGCAGAGGGGCCGGCGAGGAAGAGGGCCGAGACA
TCAGCAACGGGGCACTGGCTTTCACCCCCTTGGACACAGCTGACCCAGCCAAGTCCCCAAAGCCCAGC
AATGGGGCTGGGGTCCCCAATGGAGCCCTCCGGGAGTCCGGGGGCTCAGGCTTCCGGTCTCCCCACCT
GTGGAAGAAGTCCAGCACGCTGACCAGCAGCCGCCTAGCCACCGGCGAGGAGGAGGGCGGTGGCAGCT
CCAGCAAGCGCTTCCTGCGCTCTTGCTCCGCCTCCTGCGTTCCCCATGGGGCCAAGGACACGGAGTGG
AGGTCAGTCACGCTGCCTCGGGACTTGCAGTCCACGGGAAGACAGTTTGACTCGTCCACATTTGGAGG
GCACAAAAGTGAGAAGCCGGCTCTGCCTCGGAAGAGGGCAGGGGAGAACAGGTCTGACCAGGTGACCC
GAGGCACAGTAACGCCTCCCCCCAGGCTGGTGAAAAAGAATGAGGAAGCTGCTGATGAGGTCTTCAAA
GACATCATGGAGTCCAGCCCGGGCTCCAGCCCGCCCAACCTGACTCCAAAACCCCTCCGGCGGCAGGT
CACCGTGGCCCCTGCCTCGGGCCTCCCCCACAAGGAAGAAGCTGGAAAGGGCAGTGCCTTAGGGACCC
CTGCTGCAGCTGAGCCAGTGACCCCCACCAGCAAAGCAGGCTCAGGTGCACCAGGGGGCACCAGCAAG
GGCCCCGCCGAGGAGTCCAGAGTGAGGAGGCACAAGCACTCCTCTGAGTCGCCAGGGAGGGACAAGGG
GAAATTGTCCAGGCTCAAACCTGCCCCGCCGCCCCCACCAGCAGCCTCTGCAGGGAAGGCTGGAGGAA
AGCCCTCGCAGAGCCCGAGCCAGGAGGCGGCCGGGGAGGCAGTCCTGGGCGCAAAGACAAAAGCCACG
AGTCTGGTTGATGCTGTGAACAGTGACGCTGCCAAGCCCAGCCAGCCGGGAGAGGGCCTCAAAAAGCC
CGTGCTCCCGGCCACTCCAAAGCCACAGTCCGCCAAGCCGTCGGGGACCCCCATCAGCCCAGCCCCCG
TTCCCTCCACGTTGCCATCAGCATCCTCGGCCCTGGCAGGGGACCAGCCGTCTTCCACCGCCTTCATC
CCTCTCATATCAACCCGAGTGTCTCTTCGGAAAACCCGCCAGCCTCCAGAGCGGATCGCCAGCGGCGC
CATCACCAAGGGCGTGGTCCTGGACAGCACCGAGGCGCTGTGCCTCGCCATCTCTAGGAACTCCGAGC
AGATGGCCAGCCACAGCGCAGTGCTGGAGGCCGGCAAAAACCTCTACACGTTCTGCGTGAGCTATGTG
GATTCCATCCAGCAAATGAGGAACAAGTTTGCCTTCCGAGAGGCCATCAACAAACTGGAGAATAATCT
CCGGGAGCTTCAGATCTGCCCGGCGACAGCAGGCAGTGGTCCAGCGGCCACTCAGGACTTCAGCAAGC
TCCTCAGTTCGGTGAAGGAAATCAGTGACATAGTGCAGAGGTAG

In some embodiments, an ABL1 fusion nucleic acid molecule of the disclosure is a BCR-ABL1 fusion nucleic acid molecule, wherein the BCR-ABL1 fusion nucleic acid molecule encodes a BCR-ABL1 fusion polypeptide comprising the amino acid sequence of SEQ ID NO: 38, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 38. In some embodiments, the BCR-ABL1 fusion nucleic acid molecule comprises the nucleotide sequence of SEQ ID NO: 17, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 17.

(SEQ ID NO: 17)
ATGGTGGACCCGGTGGGCTTCGCGGAGGCGTGGAAGGCGCAGTTCCCGGACTCAGAGCCCCCGCGCAT
GGAGCTGCGCTCAGTGGGCGACATCGAGCAGGAGCTGGAGCGCTGCAAGGCCTCCATTCGGCGCCTGG
AGCAGGAGGTGAACCAGGAGCGCTTCCGCATGATCTACCTGCAGACGTTGCTGGCCAAGGAAAAGAAG
AGCTATGACCGGCAGCGATGGGGCTTCCGGCGCGCGGCGCAGGCCCCCGACGGCGCCTCCGAGCCCCG
AGCGTCCGCGTCGCGCCCGCAGCCAGCGCCCGCCGACGGAGCCGACCCGCCGCCCGCCGAGGAGCCCG
AGGCCCGGCCCGACGGCGAGGGTTCTCCGGGTAAGGCCAGGCCCGGGACCGCCCGCAGGCCCGGGGCA
GCCGCGTCGGGGGAACGGGACGACCGGGGACCCCCCGCCAGCGTGGCGGCGCTCAGGTCCAACTTCGA
GCGGATCCGCAAGGGCCATGGCCAGCCCGGGGCGGACGCCGAGAAGCCCTTCTACGTGAACGTCGAGT
TTCACCACGAGCGCGGCCTGGTGAAGGTCAACGACAAAGAGGTGTCGGACCGCATCAGCTCCCTGGGC
AGCCAGGCCATGCAGATGGAGCGCAAAAAGTCCCAGCACGGCGCGGGCTCGAGCGTGGGGGATGCATC
CAGGCCCCCTTACCGGGGACGCTCCTCGGAGAGCAGCTGCGGCGTCGACGGCGACTACGAGGACGCCG
AGTTGAACCCCCGCTTCCTGAAGGACAACCTGATCGACGCCAATGGCGGTAGCAGGCCCCCTTGGCCG
CCCCTGGAGTACCAGCCCTACCAGAGCATCTACGTCGGGGGCATGATGGAAGGGGAGGGCAAGGGCCC
GCTCCTGCGCAGCCAGAGCACCTCTGAGCAGGAGAAGCGCCTTACCTGGCCCCGCAGGTCCTACTCCC
CCCGGAGTTTTGAGGATTGCGGAGGCGGCTATACCCCGGACTGCAGCTCCAATGAGAACCTCACCTCC
AGCGAGGAGGACTTCTCCTCTGGCCAGTCCAGCCGCGTGTCCCCAAGCCCCACCACCTACCGCATGTT
CCGGGACAAAAGCCGCTCTCCCTCGCAGAACTCGCAACAGTCCTTCGACAGCAGCAGTCCCCCCACGC
CGCAGTGCCATAAGCGGCACCGGCACTGCCCGGTTGTCGTGTCCGAGGCCACCATCGTGGGCGTCCGC
AAGACCGGGCAGATCTGGCCCAACGATGGCGAGGGCGCCTTCCATGGAGACGCAGATGGCTCGTTCGG
AACACCACCTGGATACGGCTGCGCTGCAGACCGGGCAGAGGAGCAGCGCCGGCACCAAGATGGGCTGC
CCTACATTGATGACTCGCCCTCCTCATCGCCCCACCTCAGCAGCAAGGGCAGGGGCAGCCGGGATGCG
CTGGTCTCGGGAGCCCTGGAGTCCACTAAAGCGAGTGAGCTGGACTTGGAAAAGGGCTTGGAGATGAG
AAAATGGGTCCTGTCGGGAATCCTGGCTAGCGAGGAGACTTACCTGAGCCACCTGGAGGCACTGCTGC
TGCCCATGAAGCCTTTGAAAGCCGCTGCCACCACCTCTCAGCCGGTGCTGACGAGTCAGCAGATCGAG
ACCATCTTCTTCAAAGTGCCTGAGCTCTACGAGATCCACAAGGAGTTCTATGATGGGCTCTTCCCCCG
CGTGCAGCAGTGGAGCCACCAGCAGCGGGTGGGCGACCTCTTCCAGAAGCTGGCCAGCCAGCTGGGTG
TGTACCGGGCCTTCGTGGACAACTACGGAGTTGCCATGGAAATGGCTGAGAAGTGCTGTCAGGCCAAT
GCTCAGTTTGCAGAAATCTCCGAGAACCTGAGAGCCAGAAGCAACAAAGATGCCAAGGATCCAACGAC
CAAGAACTCTCTGGAAACTCTGCTCTACAAGCCTGTGGACCGTGTGACGAGGAGCACGCTGGTCCTCC
ATGACTTGCTGAAGCACACTCCTGCCAGCCACCCTGACCACCCCTTGCTGCAGGACGCCCTCCGCATC
TCACAGAACTTCCTGTCCAGCATCAATGAGGAGATCACACCCCGACGGCAGTCCATGACGGTGAAGAA
GGGAGAGCACCGGCAGCTGCTGAAGGACAGCTTCATGGTGGAGCTGGTGGAGGGGGCCCGCAAGCTGC
GCCACGTCTTCCTGTTCACCGACCTGCTTCTCTGCACCAAGCTCAAGAAGCAGAGCGGAGGCAAAACG
CAGCAGTATGACTGCAAATGGTACATTCCGCTCACGGATCTCAGCTTCCAGATGGTGGATGAACTGGA
GGCAGTGCCCAACATCCCCCTGGTGCCCGATGAGGAGCTGGACGCTTTGAAGATCAAGATCTCCCAGA
TCAAGAATGACATCCAGAGAGAGAAGAGGGCGAACAAGGGCAGCAAGGCTACGGAGAGGCTGAAGAAG
AAGCTGTCGGAGCAGGAGTCACTGCTGCTGCTTATGTCTCCCAGCATGGCCTTCAGGGTGCACAGCCG
CAACGGCAAGAGTTACACGTTCCTGATCTCCTCTGACTATGAGCGTGCAGAGTGGAGGGAGAACATCC
GGGAGCAGCAGAAGAAGTGTTTCAGAAGCTTCTCCCTGACATCCGTGGAGCTGCAGATGCTGACCAAC
TCGTGTGTGAAACTCCAGACTGTCCACAGCATTCCGCTGACCATCAATAAGGAAGATGATGAGTCTCC
GGGGCTCTATGGGTTTCTGAATGTCATCGTCCACTCAGCCACTGGATTTAAGCAGAGTTCAAAAGCCC
TTCAGCGGCCAGTAGCATCTGACTTTGAGCCTCAGGGTCTGAGTGAAGCCGCTCGTTGGAACTCCAAG
GAAAACCTTCTCGCTGGACCCAGTGAAAATGACCCCAACCTTTTCGTTGCACTGTATGATTTTGTGGC
CAGTGGAGATAACACTCTAAGCATAACTAAAGGTGAAAAGCTCCGGGTCTTAGGCTATAATCACAATG
GGGAATGGTGTGAAGCCCAAACCAAAAATGGCCAAGGCTGGGTCCCAAGCAACTACATCACGCCAGTC
AACAGTCTGGAGAAACACTCCTGGTACCATGGGCCTGTGTCCCGCAATGCCGCTGAGTATCTGCTGAG
CAGCGGGATCAATGGCAGCTTCTTGGTGCGTGAGAGTGAGAGCAGTCCTGGCCAGAGGTCCATCTCGC
TGAGATACGAAGGGAGGGTGTACCATTACAGGATCAACACTGCTTCTGATGGCAAGCTCTACGTCTCC
TCCGAGAGCCGCTTCAACACCCTGGCCGAGTTGGTTCATCATCATTCAACGGTGGCCGACGGGCTCAT
CACCACGCTCCATTATCCAGCCCCAAAGCGCAACAAGCCCACTGTCTATGGTGTGTCCCCCAACTACG
ACAAGTGGGAGATGGAACGCACGGACATCACCATGAAGCACAAGCTGGGCGGGGGCCAGTACGGGGAG
GTGTACGAGGGCGTGTGGAAGAAATACAGCCTGACGGTGGCCGTGAAGACCTTGAAGGAGGACACCAT
GGAGGTGGAAGAGTTCTTGAAAGAAGCTGCAGTCATGAAAGAGATCAAACACCCTAACCTGGTGCAGC
TCCTTGGGGTCTGCACCCGGGAGCCCCCGTTCTATATCATCACTGAGTTCATGACCTACGGGAACCTC
CTGGACTACCTGAGGGAGTGCAACCGGCAGGAGGTGAACGCCGTGGTGCTGCTGTACATGGCCACTCA
GATCTCGTCAGCCATGGAGTACCTGGAGAAGAAAAACTTCATCCACAGAGATCTTGCTGCCCGAAACT
GCCTGGTAGGGGAGAACCACTTGGTGAAGGTAGCTGATTTTGGCCTGAGCAGGTTGATGACAGGGGAC
ACCTACACAGCCCATGCTGGAGCCAAGTTCCCCATCAAATGGACTGCACCCGAGAGCCTGGCCTACAA
CAAGTTCTCCATCAAGTCCGACGTCTGGGCATTTGGAGTATTGCTTTGGGAAATTGCTACCTATGGCA
TGTCCCCTTACCCGGGAATTGACCTGTCCCAGGTGTATGAGCTGCTAGAGAAGGACTACCGCATGGAG
CGCCCAGAAGGCTGCCCAGAGAAGGTCTATGAACTCATGCGAGCATGTTGGCAGTGGAATCCCTCTGA
CCGGCCCTCCTTTGCTGAAATCCACCAAGCCTTTGAAACAATGTTCCAGGAATCCAGTATCTCAGACG
AAGTGGAAAAGGAGCTGGGGAAACAAGGCGTCCGTGGGGCTGTGAGTACCTTGCTGCAGGCCCCAGAG
CTGCCCACCAAGACGAGGACCTCCAGGAGAGCTGCAGAGCACAGAGACACCACTGACGTGCCTGAGAT
GCCTCACTCCAAGGGCCAGGGAGAGAGCGATCCTCTGGACCATGAGCCTGCCGTGTCTCCATTGCTCC
CTCGAAAAGAGCGAGGTCCCCCGGAGGGCGGCCTGAATGAAGATGAGCGCCTTCTCCCCAAAGACAAA
AAGACCAACTTGTTCAGCGCCTTGATCAAGAAGAAGAAGAAGACAGCCCCAACCCCTCCCAAACGCAG
CAGCTCCTTCCGGGAGATGGACGGCCAGCCGGAGCGCAGAGGGGCCGGCGAGGAAGAGGGCCGAGACA
TCAGCAACGGGGCACTGGCTTTCACCCCCTTGGACACAGCTGACCCAGCCAAGTCCCCAAAGCCCAGC
AATGGGGCTGGGGTCCCCAATGGAGCCCTCCGGGAGTCCGGGGGCTCAGGCTTCCGGTCTCCCCACCT
GTGGAAGAAGTCCAGCACGCTGACCAGCAGCCGCCTAGCCACCGGCGAGGAGGAGGGCGGTGGCAGCT
CCAGCAAGCGCTTCCTGCGCTCTTGCTCCGCCTCCTGCGTTCCCCATGGGGCCAAGGACACGGAGTGG
AGGTCAGTCACGCTGCCTCGGGACTTGCAGTCCACGGGAAGACAGTTTGACTCGTCCACATTTGGAGG
GCACAAAAGTGAGAAGCCGGCTCTGCCTCGGAAGAGGGCAGGGGAGAACAGGTCTGACCAGGTGACCC
GAGGCACAGTAACGCCTCCCCCCAGGCTGGTGAAAAAGAATGAGGAAGCTGCTGATGAGGTCTTCAAA
GACATCATGGAGTCCAGCCCGGGCTCCAGCCCGCCCAACCTGACTCCAAAACCCCTCCGGCGGCAGGT
CACCGTGGCCCCTGCCTCGGGCCTCCCCCACAAGGAAGAAGCTGGAAAGGGCAGTGCCTTAGGGACCC
CTGCTGCAGCTGAGCCAGTGACCCCCACCAGCAAAGCAGGCTCAGGTGCACCAGGGGGCACCAGCAAG
GGCCCCGCCGAGGAGTCCAGAGTGAGGAGGCACAAGCACTCCTCTGAGTCGCCAGGGAGGGACAAGGG
GAAATTGTCCAGGCTCAAACCTGCCCCGCCGCCCCCACCAGCAGCCTCTGCAGGGAAGGCTGGAGGAA
AGCCCTCGCAGAGCCCGAGCCAGGAGGCGGCCGGGGAGGCAGTCCTGGGCGCAAAGACAAAAGCCACG
AGTCTGGTTGATGCTGTGAACAGTGACGCTGCCAAGCCCAGCCAGCCGGGAGAGGGCCTCAAAAAGCC
CGTGCTCCCGGCCACTCCAAAGCCACAGTCCGCCAAGCCGTCGGGGACCCCCATCAGCCCAGCCCCCG
TTCCCTCCACGTTGCCATCAGCATCCTCGGCCCTGGCAGGGGACCAGCCGTCTTCCACCGCCTTCATC
CCTCTCATATCAACCCGAGTGTCTCTTCGGAAAACCCGCCAGCCTCCAGAGCGGATCGCCAGCGGCGC
CATCACCAAGGGCGTGGTCCTGGACAGCACCGAGGCGCTGTGCCTCGCCATCTCTAGGAACTCCGAGC
AGATGGCCAGCCACAGCGCAGTGCTGGAGGCCGGCAAAAACCTCTACACGTTCTGCGTGAGCTATGTG
GATTCCATCCAGCAAATGAGGAACAAGTTTGCCTTCCGAGAGGCCATCAACAAACTGGAGAATAATCT
CCGGGAGCTTCAGATCTGCCCGGCGACAGCAGGCAGTGGTCCAGCGGCCACTCAGGACTTCAGCAAGC
TCCTCAGTTCGGTGAAGGAAATCAGTGACATAGTGCAGAGGTAG

In some embodiments, an ABL1 fusion nucleic acid molecule of the disclosure is a BCR-ABL1 fusion nucleic acid molecule, wherein the BCR-ABL1 fusion nucleic acid molecule encodes a BCR-ABL1 fusion polypeptide comprising the amino acid sequence of SEQ ID NO: 39, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 39. In some embodiments, the BCR-ABL1 fusion nucleic acid molecule comprises the nucleotide sequence of SEQ ID NO: 18, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 18.

(SEQ ID NO: 18)
ATGGTGGACCCGGTGGGCTTCGCGGAGGCGTGGAAGGCGCAGTTCCCGGACTCAGAGCCCCCGCGCAT
GGAGCTGCGCTCAGTGGGCGACATCGAGCAGGAGCTGGAGCGCTGCAAGGCCTCCATTCGGCGCCTGG
AGCAGGAGGTGAACCAGGAGCGCTTCCGCATGATCTACCTGCAGACGTTGCTGGCCAAGGAAAAGAAG
AGCTATGACCGGCAGCGATGGGGCTTCCGGCGCGCGGCGCAGGCCCCCGACGGCGCCTCCGAGCCCCG
AGCGTCCGCGTCGCGCCCGCAGCCAGCGCCCGCCGACGGAGCCGACCCGCCGCCCGCCGAGGAGCCCG
AGGCCCGGCCCGACGGCGAGGGTTCTCCGGGTAAGGCCAGGCCCGGGACCGCCCGCAGGCCCGGGGCA
GCCGCGTCGGGGGAACGGGACGACCGGGGACCCCCCGCCAGCGTGGCGGCGCTCAGGTCCAACTTCGA
GCGGATCCGCAAGGGCCATGGCCAGCCCGGGGCGGACGCCGAGAAGCCCTTCTACGTGAACGTCGAGT
TTCACCACGAGCGCGGCCTGGTGAAGGTCAACGACAAAGAGGTGTCGGACCGCATCAGCTCCCTGGGC
AGCCAGGCCATGCAGATGGAGCGCAAAAAGTCCCAGCACGGCGCGGGCTCGAGCGTGGGGGATGCATC
CAGGCCCCCTTACCGGGGACGCTCCTCGGAGAGCAGCTGCGGCGTCGACGGCGACTACGAGGACGCCG
AGTTGAACCCCCGCTTCCTGAAGGACAACCTGATCGACGCCAATGGCGGTAGCAGGCCCCCTTGGCCG
CCCCTGGAGTACCAGCCCTACCAGAGCATCTACGTCGGGGGCATGATGGAAGGGGAGGGCAAGGGCCC
GCTCCTGCGCAGCCAGAGCACCTCTGAGCAGGAGAAGCGCCTTACCTGGCCCCGCAGGTCCTACTCCC
CCCGGAGTTTTGAGGATTGCGGAGGCGGCTATACCCCGGACTGCAGCTCCAATGAGAACCTCACCTCC
AGCGAGGAGGACTTCTCCTCTGGCCAGTCCAGCCGCGTGTCCCCAAGCCCCACCACCTACCGCATGTT
CCGGGACAAAAGCCGCTCTCCCTCGCAGAACTCGCAACAGTCCTTCGACAGCAGCAGTCCCCCCACGC
CGCAGTGCCATAAGCGGCACCGGCACTGCCCGGTTGTCGTGTCCGAGGCCACCATCGTGGGCGTCCGC
AAGACCGGGCAGATCTGGCCCAACGATGGCGAGGGCGCCTTCCATGGAGACGCAGATGGCTCGTTCGG
AACACCACCTGGATACGGCTGCGCTGCAGACCGGGCAGAGGAGCAGCGCCGGCACCAAGATGGGCTGC
CCTACATTGATGACTCGCCCTCCTCATCGCCCCACCTCAGCAGCAAGGGCAGGGGCAGCCGGGATGCG
CTGGTCTCGGGAGCCCTGGAGTCCACTAAAGCGAGTGAGCTGGACTTGGAAAAGGGCTTGGAGATGAG
AAAATGGGTCCTGTCGGGAATCCTGGCTAGCGAGGAGACTTACCTGAGCCACCTGGAGGCACTGCTGC
TGCCCATGAAGCCTTTGAAAGCCGCTGCCACCACCTCTCAGCCGGTGCTGACGAGTCAGCAGATCGAG
ACCATCTTCTTCAAAGTGCCTGAGCTCTACGAGATCCACAAGGAGTTCTATGATGGGCTCTTCCCCCG
CGTGCAGCAGTGGAGCCACCAGCAGCGGGTGGGCGACCTCTTCCAGAAGCTGGCCAGCCAGCTGGGTG
TGTACCGGGCCTTCGTGGACAACTACGGAGTTGCCATGGAAATGGCTGAGAAGTGCTGTCAGGCCAAT
GCTCAGTTTGCAGAAATCTCCGAGAACCTGAGAGCCAGAAGCAACAAAGATGCCAAGGATCCAACGAC
CAAGAACTCTCTGGAAACTCTGCTCTACAAGCCTGTGGACCGTGTGACGAGGAGCACGCTGGTCCTCC
ATGACTTGCTGAAGCACACTCCTGCCAGCCACCCTGACCACCCCTTGCTGCAGGACGCCCTCCGCATC
TCACAGAACTTCCTGTCCAGCATCAATGAGGAGATCACACCCCGACGGCAGTCCATGACGGTGAAGAA
GGGAGAGCACCGGCAGCTGCTGAAGGACAGCTTCATGGTGGAGCTGGTGGAGGGGGCCCGCAAGCTGC
GCCACGTCTTCCTGTTCACCGACCTGCTTCTCTGCACCAAGCTCAAGAAGCAGAGCGGAGGCAAAACG
CAGCAGTATGACTGCAAATGGTACATTCCGCTCACGGATCTCAGCTTCCAGATGGTGGATGAACTGGA
GGCAGTGCCCAACATCCCCCTGGTGCCCGATGAGGAGCTGGACGCTTTGAAGATCAAGATCTCCCAGA
TCAAGAATGACATCCAGAGAGAGAAGAGGGCGAACAAGGGCAGCAAGGCTACGGAGAGGCTGAAGAAG
AAGCTGTCGGAGCAGGAGTCACTGCTGCTGCTTATGTCTCCCAGCATGGCCTTCAGGGTGCACAGCCG
CAACGGCAAGAGTTACACGTTCCTGATCTCCTCTGACTATGAGCGTGCAGAGTGGAGGGAGAACATCC
GGGAGCAGCAGAAGAAGTGTTTCAGAAGCTTCTCCCTGACATCCGTGGAGCTGCAGATGCTGACCAAC
TCGTGTGTGAAACTCCAGACTGTCCACAGCATTCCGCTGACCATCAATAAGGAAGATGATGAGTCTCC
GGGGCTCTATGGGTTTCTGAATGTCATCGTCCACTCAGCCACTGGATTTAAGCAGAGTTCAAAAGCCC
TTCAGCGGCCAGTAGCATCTGACTTTGAGCCTCAGGGTCTGAGTGAAGCCGCTCGTTGGAACTCCAAG
GAAAACCTTCTCGCTGGACCCAGTGAAAATGACCCCAACCTTTTCGTTGCACTGTATGATTTTGTGGC
CAGTGGAGATAACACTCTAAGCATAACTAAAGGTGAAAAGCTCCGGGTCTTAGGCTATAATCACAATG
GGGAATGGTGTGAAGCCCAAACCAAAAATGGCCAAGGCTGGGTCCCAAGCAACTACATCACGCCAGTC
AACAGTCTGGAGAAACACTCCTGGTACCATGGGCCTGTGTCCCGCAATGCCGCTGAGTATCTGCTGAG
CAGCGGGATCAATGGCAGCTTCTTGGTGCGTGAGAGTGAGAGCAGTCCTGGCCAGAGGTCCATCTCGC
TGAGATACGAAGGGAGGGTGTACCATTACAGGATCAACACTGCTTCTGATGGCAAGCTCTACGTCTCC
TCCGAGAGCCGCTTCAACACCCTGGCCGAGTTGGTTCATCATCATTCAACGGTGGCCGACGGGCTCAT
CACCACGCTCCATTATCCAGCCCCAAAGCGCAACAAGCCCACTGTCTATGGTGTGTCCCCCAACTACG
ACAAGTGGGAGATGGAACGCACGGACATCACCATGAAGCACAAGCTGGGCGGGGGCCAGTACGGGGAG
GTGTACGAGGGCGTGTGGAAGAAATACAGCCTGACGGTGGCCGTGAAGACCTTGAAGGAGGACACCAT
GGAGGTGGAAGAGTTCTTGAAAGAAGCTGCAGTCATGAAAGAGATCAAACACCCTAACCTGGTGCAGC
TCCTTGGGGTCTGCACCCGGGAGCCCCCGTTCTATATCATCACTGAGTTCATGACCTACGGGAACCTC
CTGGACTACCTGAGGGAGTGCAACCGGCAGGAGGTGAACGCCGTGGTGCTGCTGTACATGGCCACTCA
GATCTCGTCAGCCATGGAGTACCTGGAGAAGAAAAACTTCATCCACAGAGATCTTGCTGCCCGAAACT
GCCTGGTAGGGGAGAACCACTTGGTGAAGGTAGCTGATTTTGGCCTGAGCAGGTTGATGACAGGGGAC
ACCTACACAGCCCATGCTGGAGCCAAGTTCCCCATCAAATGGACTGCACCCGAGAGCCTGGCCTACAA
CAAGTTCTCCATCAAGTCCGACGTCTGGGCATTTGGAGTATTGCTTTGGGAAATTGCTACCTATGGCA
TGTCCCCTTACCCGGGAATTGACCTGTCCCAGGTGTATGAGCTGCTAGAGAAGGACTACCGCATGGAG
CGCCCAGAAGGCTGCCCAGAGAAGGTCTATGAACTCATGCGAGCATGTTGGCAGTGGAATCCCTCTGA
CCGGCCCTCCTTTGCTGAAATCCACCAAGCCTTTGAAACAATGTTCCAGGAATCCAGTATCTCAGACG
AAGTGGAAAAGGAGCTGGGGAAACAAGGCGTCCGTGGGGCTGTGAGTACCTTGCTGCAGGCCCCAGAG
CTGCCCACCAAGACGAGGACCTCCAGGAGAGCTGCAGAGCACAGAGACACCACTGACGTGCCTGAGAT
GCCTCACTCCAAGGGCCAGGGAGAGAGCGATCCTCTGGACCATGAGCCTGCCGTGTCTCCATTGCTCC
CTCGAAAAGAGCGAGGTCCCCCGGAGGGCGGCCTGAATGAAGATGAGCGCCTTCTCCCCAAAGACAAA
AAGACCAACTTGTTCAGCGCCTTGATCAAGAAGAAGAAGAAGACAGCCCCAACCCCTCCCAAACGCAG
CAGCTCCTTCCGGGAGATGGACGGCCAGCCGGAGCGCAGAGGGGCCGGCGAGGAAGAGGGCCGAGACA
TCAGCAACGGGGCACTGGCTTTCACCCCCTTGGACACAGCTGACCCAGCCAAGTCCCCAAAGCCCAGC
AATGGGGCTGGGGTCCCCAATGGAGCCCTCCGGGAGTCCGGGGGCTCAGGCTTCCGGTCTCCCCACCT
GTGGAAGAAGTCCAGCACGCTGACCAGCAGCCGCCTAGCCACCGGCGAGGAGGAGGGCGGTGGCAGCT
CCAGCAAGCGCTTCCTGCGCTCTTGCTCCGCCTCCTGCGTTCCCCATGGGGCCAAGGACACGGAGTGG
AGGTCAGTCACGCTGCCTCGGGACTTGCAGTCCACGGGAAGACAGTTTGACTCGTCCACATTTGGAGG
GCACAAAAGTGAGAAGCCGGCTCTGCCTCGGAAGAGGGCAGGGGAGAACAGGTCTGACCAGGTGACCC
GAGGCACAGTAACGCCTCCCCCCAGGCTGGTGAAAAAGAATGAGGAAGCTGCTGATGAGGTCTTCAAA
GACATCATGGAGTCCAGCCCGGGCTCCAGCCCGCCCAACCTGACTCCAAAACCCCTCCGGCGGCAGGT
CACCGTGGCCCCTGCCTCGGGCCTCCCCCACAAGGAAGAAGCTGGAAAGGGCAGTGCCTTAGGGACCC
CTGCTGCAGCTGAGCCAGTGACCCCCACCAGCAAAGCAGGCTCAGGTGCACCAGGGGGCACCAGCAAG
GGCCCCGCCGAGGAGTCCAGAGTGAGGAGGCACAAGCACTCCTCTGAGTCGCCAGGGAGGGACAAGGG
GAAATTGTCCAGGCTCAAACCTGCCCCGCCGCCCCCACCAGCAGCCTCTGCAGGGAAGGCTGGAGGAA
AGCCCTCGCAGAGCCCGAGCCAGGAGGCGGCCGGGGAGGCAGTCCTGGGCGCAAAGACAAAAGCCACG
AGTCTGGTTGATGCTGTGAACAGTGACGCTGCCAAGCCCAGCCAGCCGGGAGAGGGCCTCAAAAAGCC
CGTGCTCCCGGCCACTCCAAAGCCACAGTCCGCCAAGCCGTCGGGGACCCCCATCAGCCCAGCCCCCG
TTCCCTCCACGTTGCCATCAGCATCCTCGGCCCTGGCAGGGGACCAGCCGTCTTCCACCGCCTTCATC
CCTCTCATATCAACCCGAGTGTCTCTTCGGAAAACCCGCCAGCCTCCAGAGCGGATCGCCAGCGGCGC
CATCACCAAGGGCGTGGTCCTGGACAGCACCGAGGCGCTGTGCCTCGCCATCTCTAGGAACTCCGAGC
AGATGGCCAGCCACAGCGCAGTGCTGGAGGCCGGCAAAAACCTCTACACGTTCTGCGTGAGCTATGTG
GATTCCATCCAGCAAATGAGGAACAAGTTTGCCTTCCGAGAGGCCATCAACAAACTGGAGAATAATCT
CCGGGAGCTTCAGATCTGCCCGGCGACAGCAGGCAGTGGTCCAGCGGCCACTCAGGACTTCAGCAAGC
TCCTCAGTTCGGTGAAGGAAATCAGTGACATAGTGCAGAGGTAG

In some embodiments, an ABL1 fusion nucleic acid molecule of the disclosure is a NUP214-ABL1 fusion nucleic acid molecule, wherein the NUP214-ABL1 fusion nucleic acid molecule encodes a NUP214-ABL1 fusion polypeptide comprising the amino acid sequence of SEQ ID NO: 40, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 40. In some embodiments, the NUP214-ABL1 fusion nucleic acid molecule comprises the nucleotide sequence of SEQ ID NO: 19, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 19.

(SEQ ID NO: 19)
ATGGGAGACGAGATGGATGCCATGATTCCCGAGCGGGAGATGAAGGATTTTCAGTTTAGAGCGCTAAA
GAAGGTGAGAATCTTTGACTCCCCTGAGGAATTGCCCAAGGAACGCTCGAGTCTGCTTGCTGTGTCCA
ACAAATATGGTCTGGTCTTCGCTGGTGGAGCCAGTGGCTTGCAGATTTTTCCTACTAAAAATCTTCTT
ATTCAAAATAAACCCGGAGATGATCCCAACAAAATAGTTGATAAAGTCCAAGGCTTGCTAGTTCCTAT
GAAATTCCCAATCCATCACCTGGCCTTGAGCTGTGATAACCTCACACTCTCTGCGTGCATGATGTCCA
GTGAATATGGTTCCATTATTGCTTTTTTTGATGTTCGCACATTCTCAAATGAGGCTAAACAGCAAAAA
CGCCCATTTGCCTATCATAAGCTTTTGAAAGATGCAGGAGGCATGGTGATTGATATGAAGTGGAACCC
CACTGTCCCCTCCATGGTGGCAGTTTGTCTGGCTGATGGTAGTATTGCTGTCCTGCAAGTCACGGAAA
CAGTGAAAGTATGTGCAACTCTTCCTTCCACGGTAGCAGTAACCTCTGTGTGCTGGAGCCCCAAAGGA
AAGCAGCTGGCAGTGGGAAAACAGAATGGAACTGTGGTCCAGTATCTTCCTACTTTGCAGGAAAAAAA
AGTCATTCCTTGTCCTCCGTTTTATGAGTCAGATCATCCTGTCAGAGTTCTGGATGTGCTGTGGATTG
GTACCTACGTCTTCGCCATAGTGTATGCTGCTGCAGATGGGACCCTGGAAACGTCTCCAGATGTGGTG
ATGGCTCTACTACCGAAAAAAGAAGAAAAGCACCCAGAGATATTTGTGAACTTTATGGAGCCCTGTTA
TGGCAGCTGCACGGAGAGACAGCATCATTACTACCTCAGTTACATTGAGGAATGGGATTTAGTGCTGG
CAGCATCTGCGGCTTCAACAGAAGTTAGTATCCTTGCTCGACAAAGTGATCAGATTAATTGGGAATCT
TGGCTACTGGAGGATTCTAGTCGAGCTGAATTGCCTGTGACAGACAAGAGTGATGACTCCTTGCCCAT
GGGAGTTGTCGTAGACTATACAAACCAAGTGGAAATCACCATCAGTGATGAAAAGACTCTTCCTCCTG
CTCCAGTTCTCATGTTACTTTCAACAGATGGTGTGCTTTGTCCATTTTATATGATTAATCAAAATCCT
GGGGTTAAGTCTCTCATCAAAACACCAGAGCGACTTTCATTAGAAGGAGAGCGACAGCCCAAGTCACC
AGGAAGTACTCCCACTACCCCAACCTCCTCTCAAGCCCCACAGAAACTGGATGCTTCTGCAGCTGCAG
CCCCTGCCTCTCTGCCACCTTCATCACCTGCTGCTCCCATTGCCACTTTTTCTTTGCTTCCTGCTGGT
GGAGCCCCCACTGTGTTCTCCTTTGGTTCTTCATCTTTGAAGTCATCTGCTACGGTCACTGGGGAGCC
CCCTTCATATTCCAGTGGCTCCGACAGCTCCAAAGCAGCCCCAGGCCCTGGCCCATCAACCTTCTCTT
TTGTTCCCCCTTCTAAAGCCTCCCTAGCCCCCACCCCTGCAGCGTCTCCTGTGGCTCCATCAGCTGCT
TCATTCTCCTTTGGATCATCTGGTTTTAAGCCTACCCTGGAAAGCACACCAGTGCCAAGTGTGTCTGC
TCCAAATATAGCAATGAAGCCCTCCTTCCCACCCTCAACCTCTGCTGTCAAAGTCAACCTTAGTGAAA
AGTTTACTGCTGCAGCTACCTCTACTCCTGTTAGTAGCTCCCAGAGCGCACCCCCGATGTCGCCATTC
TCTTCTGCCTCCAAGCCAGCTGCTTCTGGACCACTCAGCCACCCCACACCTCTCTCAGCACCACCTAG
TTCCGTGCCATTGAAGTCCTCAGTCTTGCCCTCACCATCAGGACGATCTGCTCAGGGCAGTTCAAGCC
CAGTGCCCTCAATGGTACAGAAATCACCCAGGATAACCCCTCCAGCGGCAAAGCCAGGCTCTCCCCAG
GCAAAGTCACTTCAGCCTGCTGTTGCAGAAAAGCAGGGACATCAGTGGAAAGATTCAGATCCTGTAAT
GGCTGGAATTGGGGAGGAGATTGCACACTTTCAGAAGGAGTTGGAAGAGTTAAAAGCCCGAACTTCCA
AAGCCTGTTTCCAAGTGGGCACTTCTGAGGAGATGAAGATGCTGCGAACAGAATCAGATGACTTGCAT
ACCTTTCTTTTGGAGATTAAAGAGACCACAGAGTCGCTTCATGGAGATATAAGTAGCCTGAAAACAAC
TTTACTTGAGGGCTTTGCTGGTGTTGAGGAAGCCAGAGAACAAAATGAAAGAAATCGTGACTCTGGTT
ATCTGCATTTGCTTTATAAAAGACCACTGGATCCCAAGAGTGAAGCTCAGCTTCAGGAAATTCGGCGC
CTTCATCAGTATGTGAAATTTGCTGTCCAAGATGTGAATGATGTTCTAGACTTGGAGTGGGATCAGCA
TCTGGAACAAAAGAAAAAACAAAGGCACCTGCTTGTGCCAGAGCGAGAGACACTGTTTAACACCCTAG
CCAACAATCGGGAAATCATCAACCAACAGAGGAAGAGGCTGAATCACCTGGTGGATAGTCTTCAGCAG
CTCCGCCTTTACAAACAGACTTCCCTGTGGAGCCTGTCCTCGGCTGTTCCTTCCCAGAGCAGCATTCA
CAGTTTTGACAGTGACCTGGAAAGCCTGTGCAATGCTTTGTTGAAAACCACCATAGAATCTCACACCA
AATCCTTGCCCAAAGTACCAGCCAAACTGTCCCCCATGAAACAGGCACAACTGAGAAACTTCTTGGCC
AAGAGGAAGACCCCACCAGTGAGATCCACTGCTCCAGCCAGCCTGTCTCGATCAGCCTTTCTGTCTCA
GAGATATTATGAAGACTTGGATGAAGTCAGCTCAACGTCATCTGTCTCCCAGTCTCTGGAGAGTGAAG
ATGCACGGACGTCCTGTAAAGATGACGAGGCAGTGGTTCAGGCCCCTCGGCACGCCCCCGTGGTTCGC
ACTCCTTCCATCCAGCCCAGTCTCTTGCCCCATGCAGCACCTTTTGCTAAATCTCACCTGGTTCATGG
TTCTTCACCTGGTGTGATGGGAACTTCAGTGGCTACATCTGCTAGCAAAATTATTCCTCAAGGGGCCG
ATAGCACAATGCTTGCCACGAAAACCGTGAAACATGGTGCACCTAGTCCTTCCCACCCCATCTCAGCC
CCGCAGGCAGCTGCCGCAGCAGCACTCAGGCGGCAGATGGCCAGTCAGGCACCAGCTGTAAACACTTT
GACTGAATCAACGTTGAAGAATGTCCCTCAAGTGGTAAATGTGCAGGAATTGAAGAATAACCCTGCAA
CCCCTTCTACAGCCATGGGTTCTTCAGTGCCCTACTCCACAGCCAAAACACCTCACCCAGTGTTGACC
CCAGTGGCTGCTAACCAAGCCAAGCAGGGGTCTCTAATAAATTCCCTTAAGCCATCTGGGCCTACACC
AGCATCCGGTCAGTTATCATCTGGTGACAAAGCTTCAGGGACAGCCAAGATAGAAACAGCTGTGACTT
CAACCCCATCTGCTTCTGGGCAGTTCAGCAAGCCTTTCTCATTTTCTCCATCAGGGACTGGCTTTAAT
TTTGGGATAATCACACCAACACCGTCTTCTAATTTCACTGCTGCACAAGGGGCAACACCCTCCACTAA
AGAGTCAAGCCAGCCGGACGCATTCTCATCTGGTGGGGGAAGCAAACCTTCTTATGAGGCCATTCCTG
AAAGCTCACCTCCCTCAGGAATCACATCCGCATCAAACACCACCCCAGGAGAACCTGCCGCATCTAGC
AGCAGACCTGTGGCACCTTCTGGAACTGCTCTTTCCACCACCTCTAGTAAGCTGGAAACCCCACCGTC
CAAGCTGGGAGAGCTTCTGTTTCCAAGTTCTTTGGCTGGAGAGACTCTGGGAAGTTTTTCAGGACTGC
GGGTTGGCCAAGCAGATGATTCTACAAAACCAACCAATAAGGCTTCATCCACAAGCCTAACTAGTACC
CAGCCAACCAAGACGTCAGGCGTGCCCTCAGGGTTTAATTTTACTGCCCCCCCGGTGTTAGGGAAGCA
CACGGAGCCCCCTGTGACATCCTCTGCAACCACCACCTCAGTAGCACCACCAGCAGCCACCAGCACTT
CCTCAACTGCCGTTTTTGGCAGTCTGCCAGTCACCAGTGCAGGATCCTCTGGGGTCATCAGTTTTGGT
GGGACATCTCTAAGTGCTGGCAAGACTAGTTTTTCATTTGGAAGCCAACAGACCAATAGCACAGTGCC
CCCATCTGCCCCACCACCAACTACAGCTGCCACTCCCCTTCCAACATCATTCCCCACATTGTCATTTG
GTAGCCTCCTGAGTTCAGCAACTACCCCCTCCCTGCCTATGTCCGCTGGCAGAAGCACAGAAGAGGCC
ACTTCATCAGCTTTGCCTGAGAAGCCAGGTGACAGTGAGGTCTCAGCATCAGCAGCCTCACTTCTAGA
GGAGCAACAGTCAGCCCAGCTTCCCCAGGCTCCTCCGCAAACTTCTGACTCTGTTAAAAAAGAACCTG
TTCTTGCCCAGCCTGCAGTCAGCAACTCTGGCACTGCAGCATCTAGTACTAGTCTTGTAGCACTTTCT
GCAGAGGCTACCCCAGCCACCACGGGGGTCCCTGATGCCAGGACGGAGGCAGTACCACCTGCTTCCTC
CTTTTCTGTGCCTGGGCAGACTGCTGTCACAGCAGCTGCTATCTCAAGTGCAGGCCCTGTGGCCGTCG
AAACATCAAGTACCCCCATAGCCTCCAGCACCACGTCCATTGTTGCTCCCGGCCCATCTGCAGAGGCA
GCAGCATTTGGTACCGTCACTTCTGGCTCATCCGTCTTTGCTCAGCCTCCTGCTGCCAGTTCTAGCTC
AGCTTTCAACCAGCTCACCAACAACACAGCCACTGCCCCCTCTGCCACGCCCGTGTTTGGGCAAGTGG
CAGCCAGCACCGCACCAAGTCTGTTTGGGCAGCAGACTGGTAGCACAGCCAGCACAGCAGCTGCCACA
CCACAGGTCAGCAGCTCAGGGTTTAGCAGCCCAGCTTTTGGTACCACAGCCCCAGGGGTCTTTGGACA
GACAACCTTCGGGCAGGCCTCAGTCTTTGGGCAGTCGGCGAGCAGTGCTGCAAGTGTCTTTTCCTTCA
GTCAGCCTGGGTTCAGTTCCGTGCCTGCCTTCGGTCAGCCTGCTTCCTCCACTCCCACATCCACCAGT
GGAAGTGTCTTTGGTGCCGCCTCAAGTACCAGTAGCTCCAGTTCCTTCTCATTTGGACAGTCTTCTCC
CAACACAGGAGGGGGGCTGTTTGGCCAAAGCAACGCTCCTGCTTTTGGGCAGAGTCCTGGCTTTGGAC
AGGGAGGCTCTGTCTTTGGTGGTACCTCAGCTGCCACCACAACAGCAGCAACCTCTGGGTTCAGCTTT
TGCCAAGCTTCAGGTTTTGGGTCTAGTAATACTGGTTCTGTGTTTGGTCAAGCAGCCAGTACTGGTGG
AATAGTCTTTGGCCAGCAATCATCCTCTTCCAGTGGTAGCGTGTTTGGGTCTGGAAACACTGGAAGAG
GGGGAGGTTTCTTCAGTGGCCTTGGAGGAAAACCCAGTCAGGATGCAGCCAACAAAAACCCATTCAGC
TCGGCCAGTGGGGGCTTTGGATCCACAGCTACCTCAAATACCTCTAACCTATTTGGAAACAGTGGGGC
CAAGACATTTGGTGGATTTGCCAGCTCGTCGTTTGGAGAGCAGAAACCCACTGGCACTTTCAGCTCTG
GAGGAGGAAGTGTGGCATCCCAAGGCTTTGGGTTTTCCTCTCCAAACAAAACAGGTGGCTTCGGTGCT
GCTCCAGTGTTTGGCAGCCCTCCTACTTTTGGGGGATCCCCTGGGTTTGGAGGGGTGCCAGCATTCGG
TTCAGCCCCAGCCTTTACAAGCCCTCTGGGCTCGACGGGAGGCAAAGTGTTCGGAGAGGGCACTGCAG
CTGCCAGCGCAGGAGGATTCGGGTTTGGGAGCAGCAGCAACACCACATCCTTCGGCACGCTCGCGAGT
CAGAATGCCCCCACTTTCGGATCACTGTCCCAACAGACTTCTGGTTTTGGGACCCAGAGTAGCGGATT
CTCTGGTTTTGGATCAGGCACAGGAGGTGAAAAGCTCCGGGTCTTAGGCTATAATCACAATGGGGAAT
GGTGTGAAGCCCAAACCAAAAATGGCCAAGGCTGGGTCCCAAGCAACTACATCACGCCAGTCAACAGT
CTGGAGAAACACTCCTGGTACCATGGGCCTGTGTCCCGCAATGCCGCTGAGTATCTGCTGAGCAGCGG
GATCAATGGCAGCTTCTTGGTGCGTGAGAGTGAGAGCAGTCCTGGCCAGAGGTCCATCTCGCTGAGAT
ACGAAGGGAGGGTGTACCATTACAGGATCAACACTGCTTCTGATGGCAAGCTCTACGTCTCCTCCGAG
AGCCGCTTCAACACCCTGGCCGAGTTGGTTCATCATCATTCAACGGTGGCCGACGGGCTCATCACCAC
GCTCCATTATCCAGCCCCAAAGCGCAACAAGCCCACTGTCTATGGTGTGTCCCCCAACTACGACAAGT
GGGAGATGGAACGCACGGACATCACCATGAAGCACAAGCTGGGCGGGGGCCAGTACGGGGAGGTGTAC
GAGGGCGTGTGGAAGAAATACAGCCTGACGGTGGCCGTGAAGACCTTGAAGGAGGACACCATGGAGGT
GGAAGAGTTCTTGAAAGAAGCTGCAGTCATGAAAGAGATCAAACACCCTAACCTGGTGCAGCTCCTTG
GGGTCTGCACCCGGGAGCCCCCGTTCTATATCATCACTGAGTTCATGACCTACGGGAACCTCCTGGAC
TACCTGAGGGAGTGCAACCGGCAGGAGGTGAACGCCGTGGTGCTGCTGTACATGGCCACTCAGATCTC
GTCAGCCATGGAGTACCTGGAGAAGAAAAACTTCATCCACAGAGATCTTGCTGCCCGAAACTGCCTGG
TAGGGGAGAACCACTTGGTGAAGGTAGCTGATTTTGGCCTGAGCAGGTTGATGACAGGGGACACCTAC
ACAGCCCATGCTGGAGCCAAGTTCCCCATCAAATGGACTGCACCCGAGAGCCTGGCCTACAACAAGTT
CTCCATCAAGTCCGACGTCTGGGCATTTGGAGTATTGCTTTGGGAAATTGCTACCTATGGCATGTCCC
CTTACCCGGGAATTGACCTGTCCCAGGTGTATGAGCTGCTAGAGAAGGACTACCGCATGGAGCGCCCA
GAAGGCTGCCCAGAGAAGGTCTATGAACTCATGCGAGCATGTTGGCAGTGGAATCCCTCTGACCGGCC
CTCCTTTGCTGAAATCCACCAAGCCTTTGAAACAATGTTCCAGGAATCCAGTATCTCAGACGAAGTGG
AAAAGGAGCTGGGGAAACAAGGCGTCCGTGGGGCTGTGAGTACCTTGCTGCAGGCCCCAGAGCTGCCC
ACCAAGACGAGGACCTCCAGGAGAGCTGCAGAGCACAGAGACACCACTGACGTGCCTGAGATGCCTCA
CTCCAAGGGCCAGGGAGAGAGCGATCCTCTGGACCATGAGCCTGCCGTGTCTCCATTGCTCCCTCGAA
AAGAGCGAGGTCCCCCGGAGGGCGGCCTGAATGAAGATGAGCGCCTTCTCCCCAAAGACAAAAAGACC
AACTTGTTCAGCGCCTTGATCAAGAAGAAGAAGAAGACAGCCCCAACCCCTCCCAAACGCAGCAGCTC
CTTCCGGGAGATGGACGGCCAGCCGGAGCGCAGAGGGGCCGGCGAGGAAGAGGGCCGAGACATCAGCA
ACGGGGCACTGGCTTTCACCCCCTTGGACACAGCTGACCCAGCCAAGTCCCCAAAGCCCAGCAATGGG
GCTGGGGTCCCCAATGGAGCCCTCCGGGAGTCCGGGGGCTCAGGCTTCCGGTCTCCCCACCTGTGGAA
GAAGTCCAGCACGCTGACCAGCAGCCGCCTAGCCACCGGCGAGGAGGAGGGCGGTGGCAGCTCCAGCA
AGCGCTTCCTGCGCTCTTGCTCCGCCTCCTGCGTTCCCCATGGGGCCAAGGACACGGAGTGGAGGTCA
GTCACGCTGCCTCGGGACTTGCAGTCCACGGGAAGACAGTTTGACTCGTCCACATTTGGAGGGCACAA
AAGTGAGAAGCCGGCTCTGCCTCGGAAGAGGGCAGGGGAGAACAGGTCTGACCAGGTGACCCGAGGCA
CAGTAACGCCTCCCCCCAGGCTGGTGAAAAAGAATGAGGAAGCTGCTGATGAGGTCTTCAAAGACATC
ATGGAGTCCAGCCCGGGCTCCAGCCCGCCCAACCTGACTCCAAAACCCCTCCGGCGGCAGGTCACCGT
GGCCCCTGCCTCGGGCCTCCCCCACAAGGAAGAAGCTGGAAAGGGCAGTGCCTTAGGGACCCCTGCTG
CAGCTGAGCCAGTGACCCCCACCAGCAAAGCAGGCTCAGGTGCACCAGGGGGCACCAGCAAGGGCCCC
GCCGAGGAGTCCAGAGTGAGGAGGCACAAGCACTCCTCTGAGTCGCCAGGGAGGGACAAGGGGAAATT
GTCCAGGCTCAAACCTGCCCCGCCGCCCCCACCAGCAGCCTCTGCAGGGAAGGCTGGAGGAAAGCCCT
CGCAGAGCCCGAGCCAGGAGGCGGCCGGGGAGGCAGTCCTGGGCGCAAAGACAAAAGCCACGAGTCTG
GTTGATGCTGTGAACAGTGACGCTGCCAAGCCCAGCCAGCCGGGAGAGGGCCTCAAAAAGCCCGTGCT
CCCGGCCACTCCAAAGCCACAGTCCGCCAAGCCGTCGGGGACCCCCATCAGCCCAGCCCCCGTTCCCT
CCACGTTGCCATCAGCATCCTCGGCCCTGGCAGGGGACCAGCCGTCTTCCACCGCCTTCATCCCTCTC
ATATCAACCCGAGTGTCTCTTCGGAAAACCCGCCAGCCTCCAGAGCGGATCGCCAGCGGCGCCATCAC
CAAGGGCGTGGTCCTGGACAGCACCGAGGCGCTGTGCCTCGCCATCTCTAGGAACTCCGAGCAGATGG
CCAGCCACAGCGCAGTGCTGGAGGCCGGCAAAAACCTCTACACGTTCTGCGTGAGCTATGTGGATTCC
ATCCAGCAAATGAGGAACAAGTTTGCCTTCCGAGAGGCCATCAACAAACTGGAGAATAATCTCCGGGA
GCTTCAGATCTGCCCGGCGACAGCAGGCAGTGGTCCAGCGGCCACTCAGGACTTCAGCAAGCTCCTCA
GTTCGGTGAAGGAAATCAGTGACATAGTGCAGAGGTAG

In some embodiments, an ABL1 fusion nucleic acid molecule of the disclosure is an ABL1-AIF1L fusion nucleic acid molecule, wherein the ABL1-AIF1L fusion nucleic acid molecule encodes an ABL1-AIF1L fusion polypeptide comprising the amino acid sequence of SEQ ID NO: 41, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 41. In some embodiments, the ABL1-AIF1L fusion nucleic acid molecule comprises the nucleotide sequence of SEQ ID NO: 20, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 20.

(SEQ ID NO: 20)
ATGTTGGAGATCTGCCTGAAGCTGGTGGGCTGCAAATCCAAGAAGGGGCTGTCCTCGTCCTCCAGCTG
TTATCTGGAAGAAGCCCTTCAGCGGCCAGTAGCATCTGACTTTGAGCCTCAGGGTCTGAGTGAAGCCG
CTCGTTGGAACTCCAAGGAAAACCTTCTCGCTGGACCCAGTGAAAATGACCCCAACCTTTTCGTTGCA
CTGTATGATTTTGTGGCCAGTGGAGATAACACTCTAAGCATAACTAAAGGTGAAAAGCTCCGGGTCTT
AGGCTATAATCACAATGGGGAATGGTGTGAAGCCCAAACCAAAAATGGCCAAGGCTGGGTCCCAAGCA
ACTACATCACGCCAGTCAACAGTCTGGAGAAACACTCCTGGTACCATGGGCCTGTGTCCCGCAATGCC
GCTGAGTATCTGCTGAGCAGCGGGATCAATGGCAGCTTCTTGGTGCGTGAGAGTGAGAGCAGTCCTGG
CCAGAGGTCCATCTCGCTGAGATACGAAGGGAGGGTGTACCATTACAGGATCAACACTGCTTCTGATG
GCAAGCTCTACGTCTCCTCCGAGAGCCGCTTCAACACCCTGGCCGAGTTGGTTCATCATCATTCAACG
GTGGCCGACGGGCTCATCACCACGCTCCATTATCCAGCCCCAAAGCGCAACAAGCCCACTGTCTATGG
TGTGTCCCCCAACTACGACAAGTGGGAGATGGAACGCACGGACATCACCATGAAGCACAAGCTGGGCG
GGGGCCAGTACGGGGAGGTGTACGAGGGCGTGTGGAAGAAATACAGCCTGACGGTGGCCGTGAAGACC
TTGAAGGAGGACACCATGGAGGTGGAAGAGTTCTTGAAAGAAGCTGCAGTCATGAAAGAGATCAAACA
CCCTAACCTGGTGCAGCTCCTTGGGGTCTGCACCCGGGAGCCCCCGTTCTATATCATCACTGAGTTCA
TGACCTACGGGAACCTCCTGGACTACCTGAGGGAGTGCAACCGGCAGGAGGTGAACGCCGTGGTGCTG
CTGTACATGGCCACTCAGATCTCGTCAGCCATGGAGTACCTGGAGAAGAAAAACTTCATCCACAGAGA
TCTTGCTGCCCGAAACTGCCTGGTAGGGGAGAACCACTTGGTGAAGGTAGCTGATTTTGGCCTGAGCA
GGTTGATGACAGGGGACACCTACACAGCCCATGCTGGAGCCAAGTTCCCCATCAAATGGACTGCACCC
GAGAGCCTGGCCTACAACAAGTTCTCCATCAAGTCCGACGTCTGGGCATTTGGAGTATTGCTTTGGGA
AATTGCTACCTATGGCATGTCCCCTTACCCGGGAATTGACCTGTCCCAGGTGTATGAGCTGCTAGAGA
AGGACTACCGCATGGAGCGCCCAGAAGGCTGCCCAGAGAAGGTCTATGAACTCATGCGAGCATGTTGG
CAGTGGAATCCCTCTGACCGGCCCTCCTTTGCTGAAATCCACCAAGCCTTTGAAACAATGTTCCAGGA
ATCCAGTATCTCAGACGAAGTGGAAAAGGAGCTGGGGAAACAAGGCGTCCGTGGGGCTGTGAGTACCT
TGCTGCAGGCCCCAGAGCTGCCCACCAAGACGAGGACCTCCAGGAGAGCTGCAGAGCACAGAGACACC
ACTGACGTGCCTGAGATGCCTCACTCCAAGGGCCAGGGAGAGAGCGAGAAGTACATGGAGTTTGACCT
GAACAATGAAGGCGAGATTGACCTGATGTCTTTAAAGAGGATGATGGAGAAGCTTGGTGTCCCCAAGA
CCCACCTGGAGATGAAGAAGATGATCTCAGAGGTGACAGGAGGGGTCAGTGACACTATATCCTACCGA
GACTTTGTGAACATGATGCTGGGGAAACGGTCGGCTGTCCTCAAGTTAGTCATGATGTTTGAAGGAAA
AGCCAACGAGAGCAGCCCCAAGCCAGTTGGCCCCCCTCCAGAGAGAGACATTGCTAGCCTGCCCTGA

In some embodiments, an ABL1 fusion nucleic acid molecule of the disclosure is an RCSD1-ABL1 fusion nucleic acid molecule, wherein the RCSD1-ABL1 fusion nucleic acid molecule encodes an RCSD1-ABL1 fusion polypeptide comprising the amino acid sequence of SEQ ID NO: 42, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 42. In some embodiments, the RCSD1-ABL1 fusion nucleic acid molecule comprises the nucleotide sequence of SEQ ID NO: 21, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 21.

(SEQ ID NO: 21
ATGGAGGAAAGACCGGCAGAGACCAATGCCAATGTGGACAACTCGGCGTCCCCCTCGGTGGCCCAGCT
GGCCGGGCGGTTTAGGGAGCAGGCGGCTGCAGCCAAGGAGACACCAGCCAGTAAACCAACCCGAAGGA
AACCGCCCTGTTCCCTCCCCCTGTTCCCCCCCAAGGTAGACCTGGGCCAGAATGGTGAGGAGAAATCA
CCACCCAATGCGAGCCACCCTCCTAAATTCAAGGTCAAGAGCTCGCCTCTGATTGAGAAGCTTCAGCT
CTACGTCTCCTCCGAGAGCCGCTTCAACACCCTGGCCGAGTTGGTTCATCATCATTCAACGGTGGCCG
ACGGGCTCATCACCACGCTCCATTATCCAGCCCCAAAGCGCAACAAGCCCACTGTCTATGGTGTGTCC
CCCAACTACGACAAGTGGGAGATGGAACGCACGGACATCACCATGAAGCACAAGCTGGGCGGGGGCCA
GTACGGGGAGGTGTACGAGGGCGTGTGGAAGAAATACAGCCTGACGGTGGCCGTGAAGACCTTGAAGG
AGGACACCATGGAGGTGGAAGAGTTCTTGAAAGAAGCTGCAGTCATGAAAGAGATCAAACACCCTAAC
CTGGTGCAGCTCCTTGGGGTCTGCACCCGGGAGCCCCCGTTCTATATCATCACTGAGTTCATGACCTA
CGGGAACCTCCTGGACTACCTGAGGGAGTGCAACCGGCAGGAGGTGAACGCCGTGGTGCTGCTGTACA
TGGCCACTCAGATCTCGTCAGCCATGGAGTACCTGGAGAAGAAAAACTTCATCCACAGAGATCTTGCT
GCCCGAAACTGCCTGGTAGGGGAGAACCACTTGGTGAAGGTAGCTGATTTTGGCCTGAGCAGGTTGAT
GACAGGGGACACCTACACAGCCCATGCTGGAGCCAAGTTCCCCATCAAATGGACTGCACCCGAGAGCC
TGGCCTACAACAAGTTCTCCATCAAGTCCGACGTCTGGGCATTTGGAGTATTGCTTTGGGAAATTGCT
ACCTATGGCATGTCCCCTTACCCGGGAATTGACCTGTCCCAGGTGTATGAGCTGCTAGAGAAGGACTA
CCGCATGGAGCGCCCAGAAGGCTGCCCAGAGAAGGTCTATGAACTCATGCGAGCATGTTGGCAGTGGA
ATCCCTCTGACCGGCCCTCCTTTGCTGAAATCCACCAAGCCTTTGAAACAATGTTCCAGGAATCCAGT
ATCTCAGACGAAGTGGAAAAGGAGCTGGGGAAACAAGGCGTCCGTGGGGCTGTGAGTACCTTGCTGCA
GGCCCCAGAGCTGCCCACCAAGACGAGGACCTCCAGGAGAGCTGCAGAGCACAGAGACACCACTGACG
TGCCTGAGATGCCTCACTCCAAGGGCCAGGGAGAGAGCGATCCTCTGGACCATGAGCCTGCCGTGTCT
CCATTGCTCCCTCGAAAAGAGCGAGGTCCCCCGGAGGGCGGCCTGAATGAAGATGAGCGCCTTCTCCC
CAAAGACAAAAAGACCAACTTGTTCAGCGCCTTGATCAAGAAGAAGAAGAAGACAGCCCCAACCCCTC
CCAAACGCAGCAGCTCCTTCCGGGAGATGGACGGCCAGCCGGAGCGCAGAGGGGCCGGCGAGGAAGAG
GGCCGAGACATCAGCAACGGGGCACTGGCTTTCACCCCCTTGGACACAGCTGACCCAGCCAAGTCCCC
AAAGCCCAGCAATGGGGCTGGGGTCCCCAATGGAGCCCTCCGGGAGTCCGGGGGCTCAGGCTTCCGGT
CTCCCCACCTGTGGAAGAAGTCCAGCACGCTGACCAGCAGCCGCCTAGCCACCGGCGAGGAGGAGGGC
GGTGGCAGCTCCAGCAAGCGCTTCCTGCGCTCTTGCTCCGCCTCCTGCGTTCCCCATGGGGCCAAGGA
CACGGAGTGGAGGTCAGTCACGCTGCCTCGGGACTTGCAGTCCACGGGAAGACAGTTTGACTCGTCCA
CATTTGGAGGGCACAAAAGTGAGAAGCCGGCTCTGCCTCGGAAGAGGGCAGGGGAGAACAGGTCTGAC
CAGGTGACCCGAGGCACAGTAACGCCTCCCCCCAGGCTGGTGAAAAAGAATGAGGAAGCTGCTGATGA
GGTCTTCAAAGACATCATGGAGTCCAGCCCGGGCTCCAGCCCGCCCAACCTGACTCCAAAACCCCTCC
GGCGGCAGGTCACCGTGGCCCCTGCCTCGGGCCTCCCCCACAAGGAAGAAGCTGGAAAGGGCAGTGCC
TTAGGGACCCCTGCTGCAGCTGAGCCAGTGACCCCCACCAGCAAAGCAGGCTCAGGTGCACCAGGGGG
CACCAGCAAGGGCCCCGCCGAGGAGTCCAGAGTGAGGAGGCACAAGCACTCCTCTGAGTCGCCAGGGA
GGGACAAGGGGAAATTGTCCAGGCTCAAACCTGCCCCGCCGCCCCCACCAGCAGCCTCTGCAGGGAAG
GCTGGAGGAAAGCCCTCGCAGAGCCCGAGCCAGGAGGCGGCCGGGGAGGCAGTCCTGGGCGCAAAGAC
AAAAGCCACGAGTCTGGTTGATGCTGTGAACAGTGACGCTGCCAAGCCCAGCCAGCCGGGAGAGGGCC
TCAAAAAGCCCGTGCTCCCGGCCACTCCAAAGCCACAGTCCGCCAAGCCGTCGGGGACCCCCATCAGC
CCAGCCCCCGTTCCCTCCACGTTGCCATCAGCATCCTCGGCCCTGGCAGGGGACCAGCCGTCTTCCAC
CGCCTTCATCCCTCTCATATCAACCCGAGTGTCTCTTCGGAAAACCCGCCAGCCTCCAGAGCGGATCG
CCAGCGGCGCCATCACCAAGGGCGTGGTCCTGGACAGCACCGAGGCGCTGTGCCTCGCCATCTCTAGG
AACTCCGAGCAGATGGCCAGCCACAGCGCAGTGCTGGAGGCCGGCAAAAACCTCTACACGTTCTGCGT
GAGCTATGTGGATTCCATCCAGCAAATGAGGAACAAGTTTGCCTTCCGAGAGGCCATCAACAAACTGG
AGAATAATCTCCGGGAGCTTCAGATCTGCCCGGCGACAGCAGGCAGTGGTCCAGCGGCCACTCAGGAC
TTCAGCAAGCTCCTCAGTTCGGTGAAGGAAATCAGTGACATAGTGCAGAGGTAG

In some embodiments, an ABL1 fusion nucleic acid molecule of the disclosure is an ABL1 fusion nucleic acid molecule as listed in Table 2 in Example 1 herein, e.g., comprising the corresponding nucleotide sequence as listed in Table 2 in Example 1 herein (or a sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology thereto), or encoding the corresponding ABL1 fusion polypeptide amino acid sequence as listed in Table 2 in Example 1 herein (or a sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology thereto).

### Exemplary ABL1 Fusions

In some embodiments, an ABL1 fusion nucleic acid molecule of the disclosure is an EHMT1-ABL1 fusion nucleic acid molecule, wherein the EHMT1-ABL1 fusion nucleic acid molecule: comprises, in 5′ to 3′ direction, at least a portion of an EHMT1 gene fused to at least a portion of an ABL1 gene; comprises or results from a breakpoint within intron 4 of an EHMT1 gene and/or a breakpoint within intron 1 of an ABL1 gene; comprises a fusion between exon 4, or a portion thereof, of an EHMT1 gene and exon 2, or a portion thereof, of an ABL1 gene; comprises or results from a 5′ breakpoint within chromosomal coordinates chr9: 140630475-140630606 and/or a 3′ breakpoint within chromosomal coordinates chr9: 133729408-133729648; comprises, in 5′ to 3′ direction, exons 1-3 and exon 4, or a portion thereof, of an EHMT1 gene, fused to exon 2, or a portion thereof, and exons 3-11 of an ABL1 gene; encodes an EHMT1-ABL1 fusion polypeptide comprising the amino acid sequence of SEQ ID NO: 23, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 23; and/or comprises the nucleotide sequence of SEQ ID NO: 2, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 2. In some embodiments, the EHMT1-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a MED27-ABL1 fusion nucleic acid molecule, wherein the MED27-ABL1 fusion nucleic acid molecule: comprises, in 5′ to 3′ direction, at least a portion of a MED27 gene fused to at least a portion of an ABL1 gene; comprises or results from a breakpoint within intron 4 of a MED27 gene and/or a breakpoint within intron 4 of an ABL1 gene; comprises a fusion between exon 4, or a portion thereof, of a MED27 gene and exon 5, or a portion thereof, of an ABL1 gene; comprises or results from a 5′ breakpoint within chromosomal coordinates chr9: 134774423-134776006 and/or a 3′ breakpoint within chromosomal coordinates chr9: 133747364-133747762; comprises, in 5′ to 3′ direction, exons 1-3 and exon 4, or a portion thereof, of a MED27 gene, fused to exon 5, or a portion thereof, and exons 6-11 of an ABL1 gene; encodes a MED27-ABL1 fusion polypeptide comprising the amino acid sequence of SEQ ID NO: 24, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 24; and/or comprises the nucleotide sequence of SEQ ID NO: 3, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 3. In some embodiments, the MED27-ABL1 fusion nucleic acid molecule encodes the ABL1 kinase domain, or a portion of the ABL1 kinase domain having kinase activity.

In some embodiments, an ABL1 fusion nucleic acid molecule of the disclosure is an RFFL-ABL1 fusion nucleic acid molecule, wherein the RFFL-ABL1 fusion nucleic acid molecule: comprises, in 5′ to 3′ direction, at least a portion of an RFFL gene fused to at least a portion of an ABL1 gene; comprises a fusion between exon 5, or a portion thereof, of an RFFL gene and exon 2, or a portion thereof, of an ABL1 gene; comprises or results from a 5′ breakpoint within chromosomal coordinates chr17: 33343379-33343419 and/or a 3′ breakpoint within chromosomal coordinates chr9: 133729485-133729525; comprises, in 5′ to 3′ direction, exons 1-4 and exon 5, or a portion thereof, of an RFFL gene, fused to exon 2, or a portion thereof, and exons 3-11 of an ABL1 gene; encodes an RFFL-ABL1 fusion polypeptide comprising the amino acid sequence of SEQ ID NO: 25, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 25; and/or comprises the nucleotide sequence of SEQ ID NO: 4, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 4. In some embodiments, the RFFL-ABL1 fusion nucleic acid molecule encodes the ABL1 kinase domain, or a portion of the ABL1 kinase domain having kinase activity.

In some embodiments, an ABL1 fusion nucleic acid molecule of the disclosure is an SLC27A4-ABL1 fusion nucleic acid molecule, wherein the SLC27A4-ABL1 fusion nucleic acid molecule: comprises, in 5′ to 3′ direction, at least a portion of an SLC27A4 gene fused to at least a portion of an ABL1 gene; comprises or results from a breakpoint within intron 12 of an SLC27A4 gene and/or a breakpoint within intron 5 of an ABL1 gene; comprises a fusion between exon 12, or a portion thereof, of an SLC27A4 gene and exon 6, or a portion thereof, of an ABL1 gene; comprises or results from a 5′ breakpoint within chromosomal coordinates chr9: 131119603-131119801 and/or a 3′ breakpoint within chromosomal coordinates chr9: 133748211-133748501; comprises, in 5′ to 3′ direction, exons 1-11 and exon 12, or a portion thereof, of an SLC27A4 gene, fused to exon 6, or a portion thereof, and exons 7-11 of an ABL1 gene; encodes an SLC27A4-ABL1 fusion polypeptide comprising the amino acid sequence of SEQ ID NO: 26, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 26; and/or comprises the nucleotide sequence of SEQ ID NO: 5, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 5. In some embodiments, the SLC27A4-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 13 of a BCR gene and/or a breakpoint within intron 1 of an ABL1 gene; comprises a fusion between exon 13, or a portion thereof, of a BCR gene and exon 2, or a portion thereof, of an ABL1 gene; comprises or results from a 5′ breakpoint within chromosomal coordinates chr22: 23632033-23632321 and/or a 3′ breakpoint within chromosomal coordinates chr9: 133591666-133591902; comprises, in 5′ to 3′ direction, exons 1-12 and exon 13, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 27, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 27; and/or comprises the nucleotide sequence of SEQ ID NO: 6, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 6. In some embodiments, the BCR-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23634537-23634672 and/or a 3′ breakpoint within chromosomal coordinates chr9: 133672815-133673059; comprises, in 5′ to 3′ direction, exons 1-13 and exon 14, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 28, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 28; and/or comprises the nucleotide sequence of SEQ ID NO: 7, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 7. In some embodiments, the BCR-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23634041-23634323 and/or a 3′ breakpoint within chromosomal coordinates chr9: 133708182-133708567; comprises, in 5′ to 3′ direction, exons 1-13 and exon 14, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 29, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 29; and/or comprises the nucleotide sequence of SEQ ID NO: 8, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 8. In some embodiments, the BCR-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23632683-23632948 and/or a 3′ breakpoint within chromosomal coordinates chr9: 133613427-133613626; comprises, in 5′ to 3′ direction, exons 1-13 and exon 14, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 30, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 30; and/or comprises the nucleotide sequence of SEQ ID NO: 9, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 9. In some embodiments, the BCR-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23634011-23634460 and/or a 3′ breakpoint within chromosomal coordinates chr9: 133645693-133646017; comprises, in 5′ to 3′ direction, exons 1-13 and exon 14, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 32, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 32; and/or comprises the nucleotide sequence of SEQ ID NO: 11, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 11. In some embodiments, the BCR-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23634211-23634632 and/or a 3′ breakpoint within chromosomal coordinates chr9: 133619101-133619395; comprises, in 5′ to 3′ direction, exons 1-13 and exon 14, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 33, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 33; and/or comprises the nucleotide sequence of SEQ ID NO: 12, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 12. In some embodiments, the BCR-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 13 of a BCR gene and/or a breakpoint within intron 1 of an ABL1 gene; comprises a fusion between exon 13, or a portion thereof, of a BCR gene and exon 2, or a portion thereof, of an ABL1 gene; comprises or results from a 5′ breakpoint within chromosomal coordinates chr22: 23631722-23631762 and/or a 3′ breakpoint within chromosomal coordinates chr9: 133729484-133729524; comprises, in 5′ to 3′ direction, exons 1-12 and exon 13, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 34, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 34; and/or comprises the nucleotide sequence of SEQ ID NO: 13, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 13. In some embodiments, the BCR-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23633346-23634163 and/or a 3′ breakpoint within chromosomal coordinates chr9: 133609201-133609909; comprises, in 5′ to 3′ direction, exons 1-13 and exon 14, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 35, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 35; and/or comprises the nucleotide sequence of SEQ ID NO: 14, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 14. In some embodiments, the BCR-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23633432-23633796 and/or a 3′ breakpoint within chromosomal coordinates chr9: 133623903-133624461; comprises, in 5′ to 3′ direction, exons 1-13 and exon 14, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 36, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 36; and/or comprises the nucleotide sequence of SEQ ID NO: 15, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 15. In some embodiments, the BCR-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23633837-23634208 and/or a 3′ breakpoint within chromosomal coordinates chr9: 133665426-133665727; comprises, in 5′ to 3′ direction, exons 1-13 and exon 14, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 37, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 37; and/or comprises the nucleotide sequence of SEQ ID NO: 16, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 16. In some embodiments, the BCR-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23632858-23633068 and/or a 3′ breakpoint within chromosomal coordinates chr9: 133701595-133701846; comprises, in 5′ to 3′ direction, exons 1-13 and exon 14, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 38, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 38; and/or comprises the nucleotide sequence of SEQ ID NO: 17, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 17. In some embodiments, the BCR-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23633208-23633695 and/or a 3′ breakpoint within chromosomal coordinates chr9: 133702617-133702992; comprises, in 5′ to 3′ direction, exons 1-13 and exon 14, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 39, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 39; and/or comprises the nucleotide sequence of SEQ ID NO: 18, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 18. In some embodiments, the BCR-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a NUP214-ABL1 fusion nucleic acid molecule, wherein the NUP214-ABL1 fusion nucleic acid molecule: comprises, in 5′ to 3′ direction, at least a portion of a NUP214 gene fused to at least a portion of an ABL1 gene; comprises a fusion between exon 34, or a portion thereof, of a NUP214 gene and exon 3, or a portion thereof, of an ABL1 gene; comprises or results from a 5′ breakpoint within chromosomal coordinates chr9: 134106039-134106079 and/or a 3′ breakpoint within chromosomal coordinates chr9: 133730222-133730262; comprises, in 5′ to 3′ direction, exons 1-33 and exon 34, or a portion thereof, of a NUP214 gene, fused to exon 3, or a portion thereof, and exons 4-11 of an ABL1 gene; encodes a NUP214-ABL1 fusion polypeptide comprising the amino acid sequence of SEQ ID NO: 40, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 40; and/or comprises the nucleotide sequence of SEQ ID NO: 19, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 19. In some embodiments, the NUP214-ABL1 fusion nucleic acid molecule encodes the ABL1 kinase domain, or a portion of the ABL1 kinase domain having kinase activity.

In some embodiments, an ABL1 fusion nucleic acid molecule of the disclosure is an ABL1-AIF1L fusion nucleic acid molecule, wherein the ABL1-AIF1L fusion nucleic acid molecule: comprises, in 5′ to 3′ direction, at least a portion of an ABL1 gene fused to at least a portion of an AIF1L gene; comprises a fusion between exon 10, or a portion thereof, of an ABL1 gene and exon 4, or a portion thereof, of an AIF1L gene; comprises or results from a 5′ breakpoint within chromosomal coordinates chr9: 133755952-133755992 and/or a 3′ breakpoint within chromosomal coordinates chr9: 133989965-133990005; comprises, in 5′ to 3′ direction, exons 1-9 and exon 10, or a portion thereof, of an ABL1 gene, fused to exon 4, or a portion thereof, and exons 5-6 of an AIF1L gene; encodes an ABL1-AIF1L fusion polypeptide comprising the amino acid sequence of SEQ ID NO: 41, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 41; and/or comprises the nucleotide sequence of SEQ ID NO: 20, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 20. In some embodiments, the ABL1-AIF1L fusion nucleic acid molecule encodes the ABL1 kinase domain, or a portion of the ABL1 kinase domain having kinase activity.

In some embodiments, an ABL1 fusion nucleic acid molecule of the disclosure is an RCSD1-ABL1 fusion nucleic acid molecule, wherein the RCSD1-ABL1 fusion nucleic acid molecule: comprises, in 5′ to 3′ direction, at least a portion of an RCSD1 gene fused to at least a portion of an ABL1 gene; comprises a fusion between exon 4, or a portion thereof, of an RCSD1 gene and exon 4, or a portion thereof, of an ABL1 gene; comprises or results from a 5′ breakpoint within chromosomal coordinates chr1: 167654668-167654708 and/or a 3′ breakpoint within chromosomal coordinates chr9: 133738173-133738213; comprises, in 5′ to 3′ direction, exons 1-3 and exon 4, or a portion thereof, of an RCSD1 gene, fused to exon 4, or a portion thereof, and exons 5-11 of an ABL1 gene; encodes an RCSD1-ABL1 fusion polypeptide comprising the amino acid sequence of SEQ ID NO: 42, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 42; and/or comprises the nucleotide sequence of SEQ ID NO: 21, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 21. In some embodiments, the RCSD1-ABL1 fusion nucleic acid molecule encodes the ABL1 kinase domain, or a portion of the ABL1 kinase domain having kinase activity.

In some embodiments, an ABL1 fusion nucleic acid molecule of the disclosure is an APOOL-ABL1 fusion nucleic acid molecule, wherein the APOOL-ABL1 fusion nucleic acid molecule: comprises, in 5′ to 3′ direction, at least a portion of an APOOL gene fused to at least a portion of an ABL1 gene; comprises or results from a breakpoint within intron 2 of an APOOL gene and/or a breakpoint within exon 2 of an ABL1 gene; comprises a fusion between exon 2, or a portion thereof, of an APOOL gene and exon 2, or a portion thereof, of an ABL1 gene; comprises or results from a 5′ breakpoint within chromosomal coordinates chrX: 84302705-84302904 and/or a 3′ breakpoint within chromosomal coordinates chr9: 133729584-133729766; and/or comprises, in 5′ to 3′ direction, exon 1 and exon 2, or a portion thereof, of an APOOL gene, fused to exon 2, or a portion thereof, and exons 3-11 of an ABL1 gene. In some embodiments, the APOOL-ABL1 fusion nucleic acid molecule encodes the ABL1 kinase domain, or a portion of the ABL1 kinase domain having kinase activity.

In some embodiments, an ABL1 fusion nucleic acid molecule of the disclosure is an ABL1-DNAH2 fusion nucleic acid molecule, wherein the ABL1-DNAH2 fusion nucleic acid molecule: comprises, in 5′ to 3′ direction, at least a portion of an ABL1 gene fused to at least a portion of a DNAH2 gene; comprises a fusion between exon 10, or a portion thereof, of an ABL1 gene and exon 4, or a portion thereof, of a DNAH2 gene; comprises or results from a 5′ breakpoint within chromosomal coordinates chr9: 133755953-133755993 and/or a 3′ breakpoint within chromosomal coordinates chr17: 7636399-7636439; and/or comprises, in 5′ to 3′ direction, exons 1-9 and exon 10, or a portion thereof, of an ABL1 gene, fused to exon 4, or a portion thereof, and exons 5-85 of a DNAH2 gene. In some embodiments, the ABL1-DNAH2 fusion nucleic acid molecule encodes the ABL1 kinase domain, or a portion of the ABL1 kinase domain having kinase activity.

In some embodiments, an ABL1 fusion nucleic acid molecule of the disclosure is a RHEX/C1orf186-ABL1 fusion nucleic acid molecule, wherein the RHEX/C1orf186-ABL1 fusion nucleic acid molecule: comprises, in 5′ to 3′ direction, at least a portion of a RHEX/Clorf186-gene fused to at least a portion of an ABL1 gene; comprises a fusion between exon 1, or a portion thereof, of a RHEX/Clorf186 gene and exon 2, or a portion thereof, of an ABL1 gene; comprises or results from a 5′ breakpoint within chromosomal coordinates chr1: 206288099-206288139 and/or a 3′ breakpoint within chromosomal coordinates chr9: 133729488-133729528; and/or comprises, in 5′ to 3′ direction, exon 1, or a portion thereof, of a RHEX/Clorf186 gene, fused to exon 2, or a portion thereof, and exons 3-11 of an ABL1 gene. In some embodiments, the RHEX/C1orf186-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a RALGPS1-ABL1 fusion nucleic acid molecule, wherein the RALGPS1-ABL1 fusion nucleic acid molecule: comprises, in 5′ to 3′ direction, at least a portion of a RALGPS1 gene fused to at least a portion of an ABL1 gene; comprises or results from a breakpoint within intron 1 of a RALGPS1 gene and/or a breakpoint within intron 2 of an ABL1 gene; comprises a fusion between exon 1, or a portion thereof, of a RALGPS1 gene and exon 3, or a portion thereof, of an ABL1 gene; comprises or results from a 5′ breakpoint within chromosomal coordinates chr9: 129694023-129694282 and/or a 3′ breakpoint within chromosomal coordinates chr9: 133730118-133730529; and/or comprises, in 5′ to 3′ direction, exon 1, or a portion thereof, of a RALGPS1 gene, fused to exon 3, or a portion thereof, and exons 4-11 of an ABL1 gene. In some embodiments, the RALGPS1-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a TSC1-ABL1 fusion nucleic acid molecule, wherein the TSC1-ABL1 fusion nucleic acid molecule: comprises, in 5′ to 3′ direction, at least a portion of a TSC1 gene fused to at least a portion of an ABL1 gene; comprises a fusion between exon 19, or a portion thereof, of a TSC1 gene and exon 2, or a portion thereof, of an ABL1 gene; comprises or results from a 5′ breakpoint within chromosomal coordinates chr9:135,776,998-135,777,038 and/or a 3′ breakpoint within chromosomal coordinates chr9: 133729491-133729531; and/or comprises, in 5′ to 3′ direction, exons 1-18 and exon 19, or a portion thereof, of a TSC1 gene, fused to exon 2, or a portion thereof, and exons 3-11 of an ABL1 gene. In some embodiments, the TSC1-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a TSPAN18-ABL1 fusion nucleic acid molecule, wherein the TSPAN18-ABL1 fusion nucleic acid molecule: comprises, in 5′ to 3′ direction, at least a portion of a TSPAN18 gene fused to at least a portion of an ABL1 gene; comprises a fusion between exon 2, or a portion thereof, of a TSPAN18 gene and exon 2, or a portion thereof, of an ABL1 gene; comprises or results from a 5′ breakpoint within chromosomal coordinates chr11: 44881929-44881969 and/or a 3′ breakpoint within chromosomal coordinates chr9: 133729517-133729557; and/or comprises, in 5′ to 3′ direction, exon 1 and exon 2, or a portion thereof, of a TSPAN18 gene, fused to exon 2, or a portion thereof, and exons 3-11 of an ABL1 gene. In some embodiments, the TSPAN18-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a ZNF804B-ABL1 fusion nucleic acid molecule, wherein the ZNF804B-ABL1 fusion nucleic acid molecule: comprises, in 5′ to 3′ direction, at least a portion of a ZNF804B gene fused to at least a portion of an ABL1 gene; comprises or results from a breakpoint within intron 1 of a ZNF804B gene and/or a breakpoint within intron 1 of an ABL1 gene; comprises a fusion between exon 1, or a portion thereof, of a ZNF804B gene and exon 2, or a portion thereof, of an ABL1 gene; comprises or results from a 5′ breakpoint within chromosomal coordinates chr7: 88521265-88521397 and/or a 3′ breakpoint within chromosomal coordinates chr9: 133729414-133729519; and/or comprises, in 5′ to 3′ direction, exon 1, or a portion thereof, of a ZNF804B gene, fused to exon 2, or a portion thereof, and exons 3-11 of an ABL1 gene. In some embodiments, the ZNF804B-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 13 of a BCR gene and/or a breakpoint within intron 1 of an ABL1 gene; comprises a fusion between exon 13, or a portion thereof, of a BCR gene and exon 2, or a portion thereof, of an ABL1 gene; comprises or results from a 5′ breakpoint within chromosomal coordinates chr22: 23631687-23632492 and/or a 3′ breakpoint within chromosomal coordinates chr9: 133604425-133604577; and/or comprises, in 5′ to 3′ direction, exons 1-12 and exon 13, or a portion thereof, of a BCR gene, fused to exon 2, or a portion thereof, and exons 3-11 of an ABL1 gene. In some embodiments, the BCR-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23632288-23632656 and/or a 3′ breakpoint within chromosomal coordinates chr9: 133663291-133663682; and/or comprises, in 5′ to 3′ direction, exons 1-13 and exon 14, or a portion thereof, of a BCR gene, fused to exon 2, or a portion thereof, and exons 3-11 of an ABL1 gene. In some embodiments, the BCR-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23634171-23634647 and/or a 3′ breakpoint within chromosomal coordinates chr9: 133638886-133639512; and/or comprises, in 5′ to 3′ direction, exons 1-13 and exon 14, or a portion thereof, of a BCR gene, fused to exon 2, or a portion thereof, and exons 3-11 of an ABL1 gene. In some embodiments, the BCR-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23633910-23634678 and/or a 3′ breakpoint within chromosomal coordinates chr9: 133724778-133725351; and/or comprises, in 5′ to 3′ direction, exons 1-13 and exon 14, or a portion thereof, of a BCR gene, fused to exon 2, or a portion thereof, and exons 3-11 of an ABL1 gene. In some embodiments, the BCR-ABL1 fusion nucleic acid molecule encodes the ABL1 kinase domain, or a portion of the ABL1 kinase domain having kinase activity.

In some embodiments, an ABL1 fusion nucleic acid molecule of the disclosure is an ABL1-NUP214 fusion nucleic acid molecule, wherein the ABL1-NUP214 fusion nucleic acid molecule: comprises, in 5′ to 3′ direction, at least a portion of an ABL1 gene fused to at least a portion of a NUP214 gene; comprises a fusion between exon 1, or a portion thereof, of an ABL1 gene and exon 31, or a portion thereof, of a NUP214 gene; comprises or results from a 5′ breakpoint within chromosomal coordinates chr9: 134090583-134090623 and/or a 3′ breakpoint within chromosomal coordinates chr9: 133710830-133710870; and/or comprises, in 5′ to 3′ direction, exon 1, or a portion thereof, of an ABL1 gene, fused to exon 31, or a portion thereof, and exons 32-36 of a NUP214 gene.

In some embodiments, an ABL1 fusion nucleic acid molecule of the disclosure is an AIF1L-ABL1 fusion nucleic acid molecule, wherein the AIF1L-ABL1 fusion nucleic acid molecule: comprises, in 5′ to 3′ direction, at least a portion of an AIF1L gene fused to at least a portion of an ABL1 gene; comprises or results from a breakpoint within intron 2 of an AIF1L gene and/or a breakpoint within intron 1 of an ABL1 gene; comprises a fusion between exon 2, or a portion thereof, of an AIF1L gene and exon 2, or a portion thereof, of an ABL1 gene; comprises or results from a 5′ breakpoint within chromosomal coordinates chr9: 133978032-133978137 and/or a 3′ breakpoint within chromosomal coordinates chr9: 133729409-133729618; and/or comprises, in 5′ to 3′ direction, exon 1 and exon 2, or a portion thereof, of an AIF1L gene, fused to exon 2, or a portion thereof, and exons 3-11 of an ABL1 gene. In some embodiments, the AIF1L-ABL1 fusion nucleic acid molecule encodes the ABL1 kinase domain, or a portion of the ABL1 kinase domain having kinase activity.

In some embodiments, an ABL1 fusion nucleic acid molecule of the disclosure is an EXOSC2-ABL1 fusion nucleic acid molecule, wherein the EXOSC2-ABL1 fusion nucleic acid molecule: comprises, in 5′ to 3′ direction, at least a portion of an EXOSC2 gene fused to at least a portion of an ABL1 gene; comprises or results from a breakpoint within exon 9 of an EXOSC2 gene and/or a breakpoint within exon 1 of an ABL1 gene; comprises a fusion between exon 9, or a portion thereof, of an EXOSC2 gene and exon 1, or a portion thereof, of an ABL1 gene; comprises or results from a 5′ breakpoint within chromosomal coordinates chr9: 133579154-133579531 and/or a 3′ breakpoint within chromosomal coordinates chr9: 133589786-133589985; and/or comprises, in 5′ to 3′ direction, exons 1-8 and exon 9, or a portion thereof, of an EXOSC2 gene, fused to exon 1, or a portion thereof, and exons 2-11 of an ABL1 gene. In some embodiments, the EXOSC2-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a NUP214-ABL1 fusion nucleic acid molecule, wherein the NUP214-ABL1 fusion nucleic acid molecule: comprises, in 5′ to 3′ direction, at least a portion of a NUP214 gene fused to at least a portion of an ABL1 gene; comprises or results from a breakpoint within intron 34 of a NUP214 gene and/or a breakpoint within exon 3 of an ABL1 gene; comprises a fusion between exon 34, or a portion thereof, of a NUP214 gene and exon 3, or a portion thereof, of an ABL1 gene; comprises or results a 5′ breakpoint within chromosomal coordinates chr9: 134106080-134106120 and/or a 3′ breakpoint within chromosomal coordinates chr9: 133730195-133730235; and/or comprises, in 5′ to 3′ direction, exons 1-33 and exon 34, or a portion thereof, of a NUP214 gene, fused to exon 3, or a portion thereof, and exons 4-11 of an ABL1 gene. In some embodiments, the NUP214-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a NUP214-ABL1 fusion nucleic acid molecule, wherein the NUP214-ABL1 fusion nucleic acid molecule: comprises, in 5′ to 3′ direction, at least a portion of a NUP214 gene fused to at least a portion of an ABL1 gene; comprises a fusion between exon 34, or a portion thereof, of a NUP214 gene and exon 3, or a portion thereof, of an ABL1 gene; comprises or results from a 5′ breakpoint within chromosomal coordinates chr9: 134106030-134106070 and/or a 3′ breakpoint within chromosomal coordinates chr9: 133730218-133730258; and/or comprises, in 5′ to 3′ direction, exons 1-33 and exon 34, or a portion thereof, of a NUP214 gene, fused to exon 3, or a portion thereof, and exons 4-11 of an ABL1 gene. In some embodiments, the NUP214-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a NUP214-ABL1 fusion nucleic acid molecule, wherein the NUP214-ABL1 fusion nucleic acid molecule: comprises, in 5′ to 3′ direction, at least a portion of a NUP214 gene fused to at least a portion of an ABL1 gene; comprises a fusion between exon 34, or a portion thereof, of a NUP214 gene and exon 3, or a portion thereof, of an ABL1 gene; comprises or results from a 5′ breakpoint within chromosomal coordinates chr9: 134106046-134106086 and/or a 3′ breakpoint within chromosomal coordinates chr9: 133730203-133730243; and/or comprises, in 5′ to 3′ direction, exons 1-33 and exon 34, or a portion thereof, of a NUP214 gene, fused to exon 3, or a portion thereof, and exons 4-11 of an ABL1 gene. In some embodiments, the NUP214-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a NUP214-ABL1 fusion nucleic acid molecule, wherein the NUP214-ABL1 fusion nucleic acid molecule: comprises, in 5′ to 3′ direction, at least a portion of a NUP214 gene fused to at least a portion of an ABL1 gene; comprises a fusion between exon 34, or a portion thereof, of a NUP214 gene and exon 3, or a portion thereof, of an ABL1 gene; comprises or results from a 5′ breakpoint within chromosomal coordinates chr9: 134106028-134106068 and/or a 3′ breakpoint within chromosomal coordinates chr9: 133730234-133730274; and/or comprises, in 5′ to 3′ direction, exons 1-33 and exon 34, or a portion thereof, of a NUP214 gene, fused to exon 3, or a portion thereof, and exons 4-11 of an ABL1 gene. In some embodiments, the NUP214-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises a fusion between exon 1, or a portion thereof, of a BCR gene and exon 3, or a portion thereof, of an ABL1 gene; comprises or results from a 5′ breakpoint within chromosomal coordinates chr22: 23524272-23524312 and/or a 3′ breakpoint within chromosomal coordinates chr9: 133730218-133730258; and/or comprises, in 5′ to 3′ direction, exon 1, or a portion thereof, of a BCR gene, fused to exon 3, or a portion thereof, and exons 4-11 of an ABL1 gene. In some embodiments, the BCR-ABL1 fusion nucleic acid molecule encodes the ABL1 kinase domain, or a portion of the ABL1 kinase domain having kinase activity.

In some embodiments, an ABL1 fusion nucleic acid molecule of the disclosure is an SNX2-ABL1 fusion nucleic acid molecule, wherein the SNX2-ABL1 fusion nucleic acid molecule: comprises, in 5′ to 3′ direction, at least a portion of an SNX2 gene fused to at least a portion of an ABL1 gene; comprises a fusion between exon 3, or a portion thereof, of an SNX2 gene and exon 4, or a portion thereof, of an ABL1 gene; comprises or results from a 5′ breakpoint within chromosomal coordinates chr5: 122135451-122135491 and/or a 3′ breakpoint within chromosomal coordinates chr9: 133738222-133738262; and/or comprises, in 5′ to 3′ direction, exons 1-2 and exon 3, or a portion thereof, of an SNX2 gene, fused to exon 4, or a portion thereof, and exons 5-11 of an ABL1 gene. In some embodiments, the SNX2-ABL1 fusion nucleic acid molecule encodes the ABL1 kinase domain, or a portion of the ABL1 kinase domain having kinase activity.

In some embodiments, an ABL1 fusion nucleic acid molecule of the disclosure is an SNX2-ABL1 fusion nucleic acid molecule, wherein the SNX2-ABL1 fusion nucleic acid molecule: comprises, in 5′ to 3′ direction, at least a portion of an SNX2 gene fused to at least a portion of an ABL1 gene; comprises a fusion between exon 3, or a portion thereof, of an SNX2 gene and exon 4, or a portion thereof, of an ABL1 gene; comprises or results from a 5′ breakpoint within chromosomal coordinates chr5: 122135445-122135485 and/or a 3′ breakpoint within chromosomal coordinates chr9: 133738214-133738254; and/or comprises, in 5′ to 3′ direction, exons 1-2 and exon 3, or a portion thereof, of an SNX2 gene, fused to exon 4, or a portion thereof, and exons 5-11 of an ABL1 gene. In some embodiments, the SNX2-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 12 of a BCR gene and/or a breakpoint within intron 1 of an ABL1 gene; comprises a fusion between exon 12, or a portion thereof, of a BCR gene and exon 2, or a portion thereof, of an ABL1 gene; comprises or results from a 5′ breakpoint within chromosomal coordinates chr22: 23631693-23632188 and/or a 3′ breakpoint within chromosomal coordinates chr9: 133607683-133608245; comprises, in 5′ to 3′ direction, exons 1-11 and exon 12, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 31, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 31; and/or comprises the nucleotide sequence of SEQ ID NO: 10, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 10. In some embodiments, the BCR-ABL1 fusion nucleic acid molecule encodes the ABL1 kinase domain, or a portion of the ABL1 kinase domain having kinase activity.

In some embodiments, an ABL1 fusion nucleic acid molecule of the disclosure is a BMP2K-ABL1 fusion nucleic acid molecule, wherein the BMP2K-ABL1 fusion nucleic acid molecule: comprises in 5′ to 3′ direction, at least a portion of a BMP2K gene fused to at least a portion of an ABL1 gene; comprises a fusion between exon 15, or a portion thereof, of a BMP2K gene and exon 3, or a portion thereof, of an ABL1 gene; comprises or results from a 5′ breakpoint within chromosomal coordinates chr4: 79800022-79800062 and/or a 3′ breakpoint within chromosomal coordinates chr9: 133730205-133730245; and/or comprises, in 5′ to 3′ direction, exons 1-14 and exon 15, or a portion thereof, of a BMP2K gene, fused to exon 3, or a portion thereof, and exons 4-11 of an ABL1 gene. In some embodiments, the BMP2K-ABL1 fusion nucleic acid molecule encodes the ABL1 kinase domain, or a portion of the ABL1 kinase domain having kinase activity.

In some embodiments, an ABL1 fusion nucleic acid molecule of the disclosure comprises a fusion between an ABL1 gene, or a portion thereof, fused to a second gene, or a portion thereof, wherein the ABL1 fusion nucleic acid molecule comprises or results from an ABL1 breakpoint within exon 1 of ABL1. In some embodiments, the ABL1 fusion nucleic acid molecule comprises, in 5′ to 3′ direction, the second gene, or a portion thereof, fused to exon 1 of an ABL1 gene, or a portion thereof. In some embodiments, the ABL1 fusion nucleic acid molecule comprises a fusion between an exon or an intron, or a portion thereof, of the second gene fused to exon 1, or a portion thereof, of an ABL1 gene. In some embodiments, the ABL1 fusion nucleic acid molecule comprises or results from a breakpoint within chromosomal coordinates chr9: 133589786-133589985. In some embodiments, the ABL1 fusion nucleic acid molecule comprises, in 5′ to 3′ direction, one or more exons, or portions thereof, of the second gene, fused to exon 1, or a portion thereof, and exons 2-11 of ABL1. In some embodiments, the second gene is an EXOSC2 gene, and wherein the ABL1 fusion nucleic acid molecule: comprises a fusion between exon 9, or a portion thereof, of an EXOSC2 gene fused to exon 1, or a portion thereof, of an ABL1 gene; comprises or results from a breakpoint within exon 9 of an EXOSC2 gene and a breakpoint within exon 1 of an ABL1 gene; comprises or results from a 5′ breakpoint within chromosomal coordinates chr9: 133579154-133579531 and/or a 3′ breakpoint within chromosomal coordinates chr9: 133589786-133589985; and/or comprises, in 5′ to 3′ direction, exons 1-8 and exon 9, or a portion thereof, of an EXOSC2 gene, fused to exon 1, or a portion thereof, and exons 2-11 of an ABL1 gene.

In some embodiments, an ABL1 fusion nucleic acid molecule of the disclosure comprises a fusion between an ABL1 gene, or a portion thereof, fused to a second gene, or a portion thereof, wherein the ABL1 fusion nucleic acid molecule comprises or results from an ABL1 breakpoint within exon 2 of ABL1. In some embodiments, the ABL1 fusion nucleic acid molecule comprises, in 5′ to 3′ direction, the second gene, or a portion thereof, fused to exon 2, or a portion thereof, of an ABL1 gene. In some embodiments, the ABL1 fusion nucleic acid molecule comprises a fusion between an exon or an intron, or a portion thereof, of the second gene fused to exon 2, or a portion thereof, of an ABL1 gene. In some embodiments, the ABL1 fusion nucleic acid molecule comprises or results from a breakpoint within chromosomal coordinates chr9: 133729584-133729766. In some embodiments, the ABL1 fusion nucleic acid molecule comprises, in 5′ to 3′ direction, one or more exons, or portions thereof, of the second gene, fused to exon 2, or a portion thereof, and exons 3-11 of an ABL1 gene. In some embodiments, the second gene is an APOOL gene, wherein the ABL1 fusion nucleic acid molecule: comprises or results from a breakpoint within intron 2 of an APOOL gene, and exon 2 of ABL1; comprises a fusion between exon 2, or a portion thereof, of an APOOL gene fused to exon 2, or a portion thereof, of an ABL1 gene; comprises or results from a 5′ breakpoint within chromosomal coordinates chrX: 84302705-84302904 and/or 3′ breakpoint within chromosomal coordinates chr9: 133729584-133729766; and/or comprises, in 5′ to 3′ direction, exon 1 and exon 2, or a portion thereof of an APOOL gene, fused to exon 2, or a portion thereof, and exons 3-11 of an ABL1 gene.

In some embodiments, an ABL1 fusion nucleic acid molecule of the disclosure comprises a fusion between an ABL1 gene, or a portion thereof, fused to a second gene, or a portion thereof, wherein the ABL1 fusion nucleic acid molecule comprises or results from an ABL1 breakpoint within exon 3 of ABL1. In some embodiments, the ABL1 fusion nucleic acid molecule comprises, in 5′ to 3′ direction, the second gene, or a portion thereof, fused to exon 3, or a portion thereof, of an ABL1 gene. In some embodiments, the ABL1 fusion nucleic acid molecule comprises a fusion between an exon or an intron, or a portion thereof, of the second gene fused to exon 3, or a portion thereof, of an ABL1 gene. In some embodiments, the ABL1 fusion nucleic acid molecule comprises or results from a breakpoint within chromosomal coordinates chr9: 133730205-133730245, chr9: 133730195-133730235, chr9: 133730203-133730243, chr9: 133730234-133730274 or chr9: 133730218-133730258. In some embodiments, the ABL1 fusion nucleic acid molecule comprises, in 5′ to 3′ direction, one or more exons, or portions thereof, of the second gene, fused to exon 3, or a portion thereof, and exons 4-11 of an ABL1 gene. In some embodiments, the second gene is a NUP214 gene, wherein the ABL1 fusion nucleic acid molecule: comprises a fusion between exon 34, or a portion thereof, of a NUP214 gene fused to exon 3, or a portion thereof, of an ABL1 gene; comprises or results from a breakpoint within intron 34 of a NUP214 gene and a breakpoint within exon 3 of an ABL1 gene; comprises or results from: a 5′ breakpoint within chromosomal coordinates chr9: 134106080-134106120 and/or a 3′ breakpoint within chromosomal coordinates chr9: 133730195-133730235, a 5′ breakpoint within chromosomal coordinates chr9: 134106030-134106070 and/or a 3′ breakpoint within chromosomal coordinates chr9: 133730218-133730258, a 5′ breakpoint within chromosomal coordinates chr9: 134106046-134106086 and/or a 3′ breakpoint within chromosomal coordinates chr9: 133730203-133730243, or a 5′ breakpoint within chromosomal coordinates chr9: 134106028-134106068 and/or a 3′ breakpoint within chromosomal coordinates chr9: 133730234-133730274; and/or comprises, in 5′ to 3′ direction, exons 1-33 and exon 34, or a portion thereof, of a NUP214 gene, fused to exon 3, or a portion thereof, and exons 4-11 of an ABL1 gene. In some embodiments, the second gene is a BMP2K gene, wherein the ABL1 fusion nucleic acid molecule: comprises a fusion between exon 15, or a portion thereof, of a BMP2K gene fused to exon 3, or a portion thereof, of an ABL1 gene; comprises or results from a 5′ breakpoint within chromosomal coordinates chr4: 79800022-79800062 and/or a 3′ breakpoint within chromosomal coordinates chr9: 133730205-133730245; and/or comprises, in 5′ to 3′ direction, exons 1-14 and exon 15, or a portion thereof, of a BMP2K gene, fused to exon 3, or a portion thereof, and exons 4-11 of an ABL1 gene. In some embodiments, the second gene is a BCR gene, wherein the ABL1 fusion nucleic acid molecule: comprises a fusion between exon 1, or a portion thereof, of a BCR gene fused to exon 3, or a portion thereof, of an ABL1 gene; comprises or results from a 5′ breakpoint within chromosomal coordinates chr22: 23524272-23524312 and/or a 3′ breakpoint within chromosomal coordinates chr9: 133730218-133730258; and/or comprises, in 5′ to 3′ direction, exon 1, or a portion thereof of a BCR gene, fused to exon 3, or a portion thereof, and exons 4-11 of an ABL1 gene.

In some embodiments, an ABL1 fusion nucleic acid molecule of the disclosure comprises a fusion between an ABL1 gene, or a portion thereof, fused to a second gene, or a portion thereof, wherein the ABL1 fusion nucleic acid molecule comprises or results from an ABL1 breakpoint within exon 4 of ABL1. In some embodiments, the ABL1 fusion nucleic acid molecule comprises, in 5′ to 3′ direction, the second gene, or a portion thereof, fused to exon 4, or a portion thereof, of an ABL1 gene. In some embodiments, the ABL1 fusion nucleic acid molecule comprises a fusion between an exon or an intron, or a portion thereof, of the second gene fused to exon 4, or a portion thereof, of an ABL1 gene. In some embodiments, the ABL1 fusion nucleic acid molecule comprises or results from a breakpoint within chromosomal coordinates chr9: 133738173-133738213, chr9: 133738222-133738262, or chr9: 133738214-133738254. In some embodiments, the ABL1 fusion nucleic acid molecule comprises, in 5′ to 3′ direction, one or more exons, or portions thereof, of the second gene, fused to exon 4, or a portion thereof, and exons 5-11 of an ABL1 gene. In some embodiments, the second gene is an RCSD1 gene, wherein the ABL1 fusion nucleic acid molecule: comprises a fusion between exon 4, or a portion thereof, of an RCSD1 gene fused to exon 4, or a portion thereof, of an ABL1 gene; comprises or results from a 5′ breakpoint within chromosomal coordinates chr1: 167654668-167654708 and/or a 3′ breakpoint within chromosomal coordinates chr9: 133738173-133738213; comprises, in 5′ to 3′ direction, exons 1-3 and exon 4, or a portion thereof, of an RCSD1 gene, fused to exon 4, or a portion thereof, and exons 5-11 of an ABL1 gene; comprises the nucleotide sequence of SEQ ID NO: 21, or a nucleotide sequence having at least about 70% homology thereto; and/or encodes an ABL1 fusion polypeptide comprising the amino acid sequence of SEQ ID NO: 42, or an amino acid sequence having at least about 70% homology thereto. In some embodiments, the second gene is an SNX2 gene, wherein the ABL1 fusion nucleic acid molecule: comprises a fusion between exon 3, or a portion thereof, of an SNX2 gene fused to exon 4, or a portion thereof, of an ABL1 gene; comprises or results from: a 5′ breakpoint within chromosomal coordinates chr5: 122135451-122135491 and/or a 3′ breakpoint within chromosomal coordinates chr9: 133738222-133738262, or a 5′ breakpoint within chromosomal coordinates chr5: 122135445-122135485 and/or a 3′ breakpoint within chromosomal coordinates chr9: 133738214-133738254; and/or comprises, in 5′ to 3′ direction, exons 1-2 and exon 3, or a portion thereof of an SNX2 gene, fused to exon 4, or a portion thereof, and exons 5-11 of an ABL1 gene.

In some embodiments, an ABL1 fusion nucleic acid molecule of the disclosure comprises a fusion between an ABL1 gene, or a portion thereof, fused to a second gene, or a portion thereof, wherein the ABL1 fusion nucleic acid molecule comprises or results from an ABL1 breakpoint within exon 10 of ABL1. In some embodiments, the ABL1 fusion nucleic acid molecule comprises, in 5′ to 3′ direction, exon 10, or a portion thereof, of an ABL1 gene fused to the second gene, or a portion thereof. In some embodiments, the ABL1 fusion nucleic acid molecule comprises a fusion between an exon or an intron, or a portion thereof, of the second gene fused to exon 10, or a portion thereof, of an ABL1 gene. In some embodiments, the ABL1 fusion nucleic acid molecule comprises or results from a breakpoint within chromosomal coordinates chr9: 133755953-133755993. In some embodiments, the ABL1 fusion nucleic acid molecule comprises, in 5′ to 3′ direction, exons 1-9 and exon 10, or a portion thereof, of an ABL1 gene fused to one or more exons, or portions thereof, of the second gene. In some embodiments, the second gene is a DNAH2 gene, wherein the ABL1 fusion nucleic acid molecule: (a) comprises a fusion between exon 10, or a portion thereof, of an ABL1 gene fused to exon 4, or a portion thereof, of a DNAH2 gene; (b) comprises or results from a 5′ breakpoint within chromosomal coordinates chr9: 133755953-133755993 and/or a 3′ breakpoint within chromosomal coordinates chr17: 7636399-7636439; and/or (c) comprises, in 5′ to 3′ direction, exons 1-9 and exon 10, or a portion thereof, of an ABL1 gene fused to exon 4, or a portion thereof, and exons 5-85 of a DNAH2 gene.

In some embodiments, an ABL1 fusion nucleic acid molecule of the disclosure comprises a fusion between an ABL1 gene, or a portion thereof, fused to a second gene, or a portion thereof, wherein the ABL1 fusion nucleic acid molecule comprises or results from an ABL1 breakpoint within intron 4 of an ABL1 gene. In some embodiments, the ABL1 fusion nucleic acid molecule comprises, in 5′ to 3′ direction, the second gene, or a portion thereof, fused to exon 5, or a portion thereof, of an ABL1 gene. In some embodiments, the ABL1 fusion nucleic acid molecule comprises a fusion between an exon or an intron of the second gene, or a portion thereof, fused to intron 4, or a portion thereof, of an ABL1 gene. In some embodiments, the ABL1 fusion nucleic acid molecule comprises or results from a breakpoint within chromosomal coordinates chr9: 133747364-133747762. In some embodiments, the ABL1 fusion nucleic acid molecule comprises, in 5′ to 3′ direction, one or more exons, or portions thereof, of the second gene, fused to exon 5, or a portion thereof, and exons 6-11 of an ABL1 gene. In some embodiments, the second gene is a MED27 gene, wherein the ABL1 fusion nucleic acid molecule: comprises a fusion between exon 4, or a portion thereof, of a MED27 gene fused to exon 5, or a portion thereof, of an ABL1 gene; comprises or results from a breakpoint within intron 4 of a MED27 gene and a breakpoint within intron 4 of an ABL1 gene; comprises or results from a 5′ breakpoint within chromosomal coordinates chr9: 134774423-134776006 and/or a 3′ breakpoint within chromosomal coordinates chr9: 133747364-133747762; comprises, in 5′ to 3′ direction, exons 1-3 and exon 4, or a portion thereof of a MED27 gene, fused to exon 5, or a portion thereof, and exons 6-11 of an ABL1 gene; comprises the nucleotide sequence of SEQ ID NO: 3, or a nucleotide sequence having at least about 70% homology thereto; and/or encodes an ABL1 fusion polypeptide comprising the amino acid sequence of SEQ ID NO: 24, or an amino acid sequence having at least about 70% homology thereto.

In some embodiments, an ABL1 fusion nucleic acid molecule of the disclosure comprises a fusion between an ABL1 gene, or a portion thereof, fused to a second gene, or a portion thereof, wherein the ABL1 fusion nucleic acid molecule comprises or results from an ABL1 breakpoint within intron 5 of an ABL1 gene. In some embodiments, the ABL1 fusion nucleic acid molecule comprises, in 5′ to 3′ direction, the second gene, or a portion thereof, fused to exon 6, or a portion thereof, of an ABL1 gene. In some embodiments, the ABL1 fusion nucleic acid molecule comprises a fusion between an exon or an intron, or a portion thereof, of the second gene fused to intron 5, or a portion thereof, of an ABL1 gene. In some embodiments, the ABL1 fusion nucleic acid molecule comprises or results from a breakpoint within chromosomal coordinates chr9: 133748211-133748501. In some embodiments, the ABL1 fusion nucleic acid molecule comprises, in 5′ to 3′ direction, one or more exons, or portions thereof, of the second gene, fused to exon 6, or a portion thereof, and exons 7-11 of an ABL1 gene. In some embodiments, the second gene is an SLC27A4 gene, wherein the ABL1 fusion nucleic acid molecule: comprises a fusion between exon 12, or a portion thereof, of a SLC27A4 gene fused to exon 6, or a portion thereof, of an ABL1 gene; comprises or results from a breakpoint within intron 12 of a SLC27A4 gene and a breakpoint within intron 5 of an ABL1 gene; comprises or results from a 5′ breakpoint within chromosomal coordinates chr9: 131119603-131119801 and/or a 3′ breakpoint within chromosomal coordinates chr9: 133748211-133748501; comprises, in 5′ to 3′ direction, exons 1-11 and exon 12, or a portion thereof, of an SLC27A4 gene, fused to exon 6, or a portion thereof, and exons 7-11 of an ABL1 gene; comprises the nucleotide sequence of SEQ ID NO: 5, or a nucleotide sequence having at least about 70% homology thereto; and/or encodes an ABL1 fusion polypeptide comprising the amino acid sequence of SEQ ID NO: 26, or an amino acid sequence having at least about 70% homology thereto.

In some embodiments of any of the ABL1 fusion nucleic acid molecules provided herein, the ABL1 fusion nucleic acid molecule is a genomic nucleic acid molecule (i.e., genomic DNA or fragments thereof), or a transcribed nucleic acid molecule, e.g., an RNA such as mRNA, or a cDNA, or fragments thereof.

In some embodiments of any of the ABL1 fusion nucleic acid molecules provided herein, the chromosomal coordinates corresponding to any of the breakpoints described herein correspond to Homo sapiens (human) genome assembly GRCh37 (hg19).

### (ii) ABL1 Fusion Polypeptides

In certain aspects, provided herein are ABL1 fusion polypeptides which comprise at least a portion of an ABL1 polypeptide and at least a portion of a polypeptide encoded by another gene.

In some embodiments, an ABL1 fusion polypeptide of the disclosure is a fusion polypeptide encoded by any of the ABL1 fusion nucleic acid molecules provided herein, or a portion thereof.

### Exemplary ABL1 Fusion Polypeptide Partners

In some embodiments, an ABL1 fusion polypeptide of the disclosure is an EHMT1-ABL1 fusion polypeptide encoded by an ABL1 fusion nucleic acid molecule comprising, in 5′ to 3′ direction, at least a portion of an EHMT1 gene fused to at least a portion of an ABL1 gene. In some embodiments, an ABL1 fusion polypeptide of the disclosure is a MED27-ABL1 fusion polypeptide encoded by an ABL1 fusion nucleic acid molecule comprising, in 5′ to 3′ direction, at least a portion of a MED27 gene fused to at least a portion of an ABL1 gene. In some embodiments, an ABL1 fusion polypeptide of the disclosure is an RFFL-ABL1 fusion polypeptide encoded by an ABL1 fusion nucleic acid molecule comprising, in 5′ to 3′ direction, at least a portion of an RFFL gene fused to at least a portion of an ABL1 gene. In some embodiments, an ABL1 fusion polypeptide of the disclosure is an SLC27A4-ABL1 fusion polypeptide encoded by an ABL1 fusion nucleic acid molecule comprising, in 5′ to 3′ direction, at least a portion of an SLC27A4 gene fused to at least a portion of an ABL1 gene. In some embodiments, an ABL1 fusion polypeptide of the disclosure is a BCR-ABL1 fusion polypeptide encoded by an ABL1 fusion nucleic acid molecule comprising, in the 5′ to 3′ direction, at least a portion of a BCR gene fused to at least a portion of an ABL1 gene. In some embodiments, an ABL1 fusion polypeptide of the disclosure is a NUP214-ABL1 fusion polypeptide encoded by an ABL1 fusion nucleic acid molecule comprising, in 5′ to 3′ direction, at least a portion of a NUP214 gene fused to at least a portion of an ABL1 gene. In some embodiments, an ABL1 fusion polypeptide of the disclosure is an ABL1-AIF1L fusion polypeptide encoded by an ABL1 fusion nucleic acid molecule comprising, in 5′ to 3′ direction, at least a portion of an ABL1 gene fused to at least a portion of an AIF1L gene. In some embodiments, an ABL1 fusion polypeptide of the disclosure is an RCSD1-ABL1 fusion polypeptide encoded by an ABL1 fusion nucleic acid molecule comprising, in 5′ to 3′ direction, at least a portion of an RCSD1 gene fused to at least a portion of an ABL1 gene. In some embodiments, an ABL1 fusion polypeptide of the disclosure is an APOOL-ABL1 fusion polypeptide encoded by an ABL1 fusion nucleic acid molecule comprising, in the 5′ to 3′ direction, at least a portion of an APOOL gene fused to at least a portion of an ABL1 gene. In some embodiments, an ABL1 fusion polypeptide of the disclosure is an ABL1-DNAH2 fusion polypeptide encoded by an ABL1 fusion nucleic acid molecule comprising, in 5′ to 3′ direction, at least a portion of an ABL1 gene fused to at least a portion of a DNAH2 gene. In some embodiments, an ABL1 fusion polypeptide of the disclosure is a RHEX/C1orf186-ABL1 fusion polypeptide encoded by an ABL1 fusion nucleic acid molecule comprising, in 5′ to 3′ direction, at least a portion of a RHEX/C1orf186-gene fused to at least a portion of an ABL1 gene. In some embodiments, an ABL1 fusion polypeptide of the disclosure is a RALGPS1-ABL1 fusion polypeptide encoded by an ABL1 fusion nucleic acid molecule comprising, in 5′ to 3′ direction, at least a portion of a RALGPS1 gene fused to at least a portion of an ABL1 gene. In some embodiments, an ABL1 fusion polypeptide of the disclosure is a TSC1-ABL1 fusion polypeptide encoded by an ABL1 fusion nucleic acid molecule comprising, in 5′ to 3′ direction, at least a portion of a TSC1 gene fused to at least a portion of an ABL1 gene. In some embodiments, an ABL1 fusion polypeptide of the disclosure is a TSPAN18-ABL1 fusion polypeptide encoded by an ABL1 fusion nucleic acid molecule comprising, in 5′ to 3′ direction, at least a portion of a TSPAN18 gene fused to at least a portion of an ABL1 gene. In some embodiments, an ABL1 fusion polypeptide of the disclosure is a ZNF804B-ABL1 fusion polypeptide encoded by an ABL1 fusion nucleic acid molecule comprising, in 5′ to 3′ direction, at least a portion of a ZNF804B gene fused to at least a portion of an ABL1 gene. In some embodiments, an ABL1 fusion polypeptide of the disclosure is an ABL1-NUP214 fusion polypeptide encoded by an ABL1 fusion nucleic acid molecule comprising, in 5′ to 3′ direction, at least a portion of an ABL1 gene fused to at least a portion of a NUP214 gene. In some embodiments, an ABL1 fusion polypeptide of the disclosure is an AIF1L-ABL1 fusion polypeptide encoded by an ABL1 fusion nucleic acid molecule comprising, in 5′ to 3′ direction, at least a portion of an AIF1L gene fused to at least a portion of an ABL1 gene. In some embodiments, an ABL1 fusion polypeptide of the disclosure is an EXOSC2-ABL1 fusion polypeptide encoded by an ABL1 fusion nucleic acid molecule comprising, in 5′ to 3′ direction, at least a portion of an EXOSC2 gene fused to at least a portion of an ABL1 gene. In some embodiments, an ABL1 fusion polypeptide of the disclosure is an SNX2-ABL1 fusion polypeptide encoded by an ABL1 fusion nucleic acid molecule comprising, in 5′ to 3′ direction, at least a portion of an SNX2 gene fused to at least a portion of an ABL1 gene. In some embodiments, an ABL1 fusion polypeptide of the disclosure is a BMP2K-ABL1 fusion polypeptide encoded by an ABL1 fusion nucleic acid molecule comprising, in 5′ to 3′ direction, at least a portion of a BMP2K gene fused to at least a portion of an ABL1 gene.

In some embodiments, an ABL1 fusion polypeptide of the disclosure is an EHMT1-ABL1 fusion polypeptide, comprising, in the N- to C-terminus direction, at least a portion of an EHMT1 polypeptide fused to at least a portion of an ABL1 polypeptide. In some embodiments, an ABL1 fusion polypeptide of the disclosure is a MED27-ABL1 fusion polypeptide, comprising, in the N- to C-terminus direction, at least a portion of a MED27 polypeptide fused to at least a portion of an ABL1 polypeptide. In some embodiments, an ABL1 fusion polypeptide of the disclosure is an RFFL-ABL1 fusion polypeptide, comprising, in the N- to C-terminus direction, at least a portion of an RFFL polypeptide fused to at least a portion of an ABL1 polypeptide. In some embodiments, an ABL1 fusion polypeptide of the disclosure is an SLC27A4-ABL1 fusion polypeptide, comprising, in the N- to C-terminus direction, at least a portion of an SLC27A4 polypeptide fused to at least a portion of an ABL1 polypeptide. In some embodiments, an ABL1 fusion polypeptide of the disclosure is a BCR-ABL1 fusion polypeptide, comprising, in the N- to C-terminus direction, at least a portion of a BCR polypeptide fused to at least a portion of an ABL1 polypeptide. In some embodiments, an ABL1 fusion polypeptide of the disclosure is a NUP214-ABL1 fusion polypeptide, comprising, in the N- to C-terminus direction, at least a portion of a NUP214 polypeptide fused to at least a portion of an ABL1 polypeptide. In some embodiments, an ABL1 fusion polypeptide of the disclosure is an ABL1-AIF1L fusion polypeptide, comprising, in the N- to C-terminus direction, at least a portion of an ABL1 polypeptide fused to at least a portion of an AIF1L polypeptide. In some embodiments, an ABL1 fusion polypeptide of the disclosure is an RCSD1-ABL1 fusion polypeptide, comprising, in the N- to C-terminus direction, at least a portion of an RCSD1 polypeptide fused to at least a portion of an ABL1 polypeptide. In some embodiments, an ABL1 fusion polypeptide of the disclosure is an APOOL-ABL1 fusion polypeptide, comprising, in the N- to C-terminus direction, at least a portion of an APOOL polypeptide fused to at least a portion of an ABL1 polypeptide. In some embodiments, an ABL1 fusion polypeptide of the disclosure is an ABL1-DNAH2 fusion polypeptide, comprising, in the N- to C-terminus direction, at least a portion of an ABL1 polypeptide fused to at least a portion of a DNAH2 polypeptide. In some embodiments, an ABL1 fusion polypeptide of the disclosure is a RHEX/C1orf186-ABL1 fusion polypeptide, comprising, in the N- to C-terminus direction, at least a portion of a RHEX/C1orf186-polypeptide fused to at least a portion of an ABL1 polypeptide. In some embodiments, an ABL1 fusion polypeptide of the disclosure is a RALGPS1-ABL1 fusion polypeptide, comprising, in the N- to C-terminus direction, at least a portion of a RALGPS1 polypeptide fused to at least a portion of an ABL1 polypeptide. In some embodiments, an ABL1 fusion polypeptide of the disclosure is a TSC1-ABL1 fusion polypeptide, comprising, in the N- to C-terminus direction, at least a portion of a TSC1 polypeptide fused to at least a portion of an ABL1 polypeptide. In some embodiments, an ABL1 fusion polypeptide of the disclosure is a TSPAN18-ABL1 fusion polypeptide, comprising, in the N- to C-terminus direction, at least a portion of a TSPAN18 polypeptide fused to at least a portion of an ABL1 polypeptide. In some embodiments, an ABL1 fusion polypeptide of the disclosure is a ZNF804B-ABL1 fusion polypeptide, comprising, in the N- to C-terminus direction, at least a portion of a ZNF804B polypeptide fused to at least a portion of an ABL1 polypeptide. In some embodiments, an ABL1 fusion polypeptide of the disclosure is an ABL1-NUP214 fusion polypeptide, comprising, in the N- to C-terminus direction, at least a portion of an ABL1 polypeptide fused to at least a portion of a NUP214 polypeptide. In some embodiments, an ABL1 fusion polypeptide of the disclosure is an AIF1L-ABL1 fusion polypeptide, comprising, in the N- to C-terminus direction, at least a portion of an AIF1L polypeptide fused to at least a portion of an ABL1 polypeptide. In some embodiments, an ABL1 fusion polypeptide of the disclosure is an EXOSC2-ABL1 fusion polypeptide, comprising, in the N- to C-terminus direction, at least a portion of an EXOSC2 polypeptide fused to at least a portion of an ABL1 polypeptide. In some embodiments, an ABL1 fusion polypeptide of the disclosure is an SNX2-ABL1 fusion polypeptide, comprising, in the N- to C-terminus direction, at least a portion of an SNX2 polypeptide fused to at least a portion of an ABL1 polypeptide. In some embodiments, an ABL1 fusion polypeptide of the disclosure is a BMP2K-ABL1 fusion polypeptide, comprising, in the N- to C-terminus direction, at least a portion of a BMP2K polypeptide fused to at least a portion of an ABL1 polypeptide.

In some embodiments, an ABL1 fusion polypeptide of the disclosure comprises at least a portion of an ABL1 polypeptide and at least a portion of a fusion partner polypeptide as provided in Table 1 or Table 2 of Example 1 herein.

### Exemplary ABL1 Fusion Breakpoints

In some embodiments, an ABL1 fusion polypeptide of the disclosure is an EHMT1-ABL1 fusion polypeptide encoded by an ABL1 fusion nucleic acid molecule comprising or resulting from a breakpoint within intron 4 of an EHMT1 gene and/or a breakpoint within intron 1 of an ABL1 gene. In some embodiments, an ABL1 fusion polypeptide of the disclosure is a MED27-ABL1 fusion polypeptide encoded by an ABL1 fusion nucleic acid molecule comprising or resulting from a breakpoint within intron 4 of a MED27 gene and/or a breakpoint within intron 4 of an ABL1 gene. In some embodiments, an ABL1 fusion polypeptide of the disclosure is an SLC27A4-ABL1 fusion polypeptide encoded by an ABL1 fusion nucleic acid molecule comprising or resulting from a breakpoint within intron 12 of an SLC27A4 gene and/or a breakpoint within intron 5 of an ABL1 gene. In some embodiments, an ABL1 fusion polypeptide of the disclosure is a BCR-ABL1 fusion polypeptide encoded by an ABL1 fusion nucleic acid molecule comprising or resulting from a breakpoint within intron 13 of a BCR gene and/or a breakpoint within intron 1 of an ABL1 gene. In some embodiments, an ABL1 fusion polypeptide of the disclosure is a BCR-ABL1 fusion polypeptide encoded by an ABL1 fusion nucleic acid molecule comprising or resulting from a breakpoint within intron 14 of a BCR gene and/or a breakpoint within intron 1 of an ABL1 gene. In some embodiments, an ABL1 fusion polypeptide of the disclosure is a BCR-ABL1 fusion polypeptide encoded by an ABL1 fusion nucleic acid molecule comprising or resulting from a breakpoint within intron 12 of a BCR gene and/or a breakpoint within intron 1 of an ABL1 gene. In some embodiments, an ABL1 fusion polypeptide of the disclosure is an APOOL-ABL1 fusion polypeptide encoded by an ABL1 fusion nucleic acid molecule comprising or resulting from a breakpoint within intron 2 of an APOOL gene and/or a breakpoint within exon 2 of an ABL1 gene. In some embodiments, an ABL1 fusion polypeptide of the disclosure is a RALGPS1-ABL1 fusion polypeptide encoded by an ABL1 fusion nucleic acid molecule comprising or resulting from a breakpoint within intron 1 of a RALGPS1 gene and/or a breakpoint within intron 2 of an ABL1 gene. In some embodiments, an ABL1 fusion polypeptide of the disclosure is a ZNF804B-ABL1 fusion polypeptide encoded by an ABL1 fusion nucleic acid molecule comprising or resulting from a breakpoint within intron 1 of a ZNF804B gene and/or a breakpoint within intron 1 of an ABL1 gene. In some embodiments, an ABL1 fusion polypeptide of the disclosure is an AIF1L-ABL1 fusion polypeptide encoded by an ABL1 fusion nucleic acid molecule comprising or resulting from a breakpoint within intron 2 of an AIF1L gene and/or a breakpoint within intron 1 of an ABL1 gene. In some embodiments, an ABL1 fusion polypeptide of the disclosure is an EXOSC2-ABL1 fusion polypeptide encoded by an ABL1 fusion nucleic acid molecule comprising or resulting from a breakpoint within exon 9 of an EXOSC2 gene and/or a breakpoint within exon 1 of an ABL1 gene. In some embodiments, an ABL1 fusion polypeptide of the disclosure is a NUP214-ABL1 fusion polypeptide encoded by an ABL1 fusion nucleic acid molecule comprising or resulting from a breakpoint within intron 34 of a NUP214 gene and/or a breakpoint within exon 3 of an ABL1 gene.

In some embodiments, an ABL1 fusion polypeptide of the disclosure is an EHMT1-ABL1 fusion polypeptide encoded by an EHMT1-ABL1 fusion nucleic acid molecule comprising or resulting from a 5′ breakpoint within chromosomal coordinates chr9: 140630475-140630606 and/or a 3′ breakpoint within chromosomal coordinates chr9: 133729408-133729648. In some embodiments, an ABL1 fusion polypeptide of the disclosure is a MED27-ABL1 fusion polypeptide encoded by a MED27-ABL1 fusion nucleic acid molecule comprising or resulting from a 5′ breakpoint within chromosomal coordinates chr9: 134774423-134776006 and/or a 3′ breakpoint within chromosomal coordinates chr9: 133747364-133747762. In some embodiments, an ABL1 fusion polypeptide of the disclosure is a RFFL-ABL1 fusion polypeptide encoded by an RFFL-ABL1 fusion nucleic acid molecule comprising or resulting from a 5′ breakpoint within chromosomal coordinates chr17: 33343379-33343419 and/or a 3′ breakpoint within chromosomal coordinates chr9: 133729485-133729525. In some embodiments, an ABL1 fusion polypeptide of the disclosure is a SLC27A4-ABL1 fusion polypeptide encoded by an SLC27A4-ABL1 fusion nucleic acid molecule comprising or resulting from a 5′ breakpoint within chromosomal coordinates chr9: 131119603-131119801 and/or a 3′ breakpoint within chromosomal coordinates chr9: 133748211-133748501. In some embodiments, an ABL1 fusion polypeptide of the disclosure is a BCR-ABL1 fusion polypeptide encoded by a BCR-ABL1 fusion nucleic acid molecule comprising or resulting from a 5′ breakpoint within chromosomal coordinates chr22: 23632033-23632321 and/or a 3′ breakpoint within chromosomal coordinates chr9: 133591666-133591902. In some embodiments, an ABL1 fusion polypeptide of the disclosure is a BCR-ABL1 fusion polypeptide encoded by a BCR-ABL1 fusion nucleic acid molecule comprising or resulting from a 5′ breakpoint within chromosomal coordinates chr22: 23634537-23634672 and/or a 3′ breakpoint within chromosomal coordinates chr9: 133672815-133673059. In some embodiments, an ABL1 fusion polypeptide of the disclosure is a BCR-ABL1 fusion polypeptide encoded by a BCR-ABL1 fusion nucleic acid molecule comprising or resulting from a 5′ breakpoint within chromosomal coordinates chr22: 23634041-23634323 and/or a 3′ breakpoint within chromosomal coordinates chr9: 133708182-133708567. In some embodiments, an ABL1 fusion polypeptide of the disclosure is a BCR-ABL1 fusion polypeptide encoded by a BCR-ABL1 fusion nucleic acid molecule comprising or resulting from a 5′ breakpoint within chromosomal coordinates chr22: 23632683-23632948 and/or a 3′ breakpoint within chromosomal coordinates chr9: 133613427-133613626. In some embodiments, an ABL1 fusion polypeptide of the disclosure is a BCR-ABL1 fusion polypeptide encoded by a BCR-ABL1 fusion nucleic acid molecule comprising or resulting from a 5′ breakpoint within chromosomal coordinates chr22: 23634011-23634460 and/or a 3′ breakpoint within chromosomal coordinates chr9: 133645693-133646017. In some embodiments, an ABL1 fusion polypeptide of the disclosure is a BCR-ABL1 fusion polypeptide encoded by a BCR-ABL1 fusion nucleic acid molecule comprising or resulting from a 5′ breakpoint within chromosomal coordinates chr22: 23634211-23634632 and/or a 3′ breakpoint within chromosomal coordinates chr9: 133619101-133619395. In some embodiments, an ABL1 fusion polypeptide of the disclosure is a BCR-ABL1 fusion polypeptide encoded by a BCR-ABL1 fusion nucleic acid molecule comprising or resulting from a 5′ breakpoint within chromosomal coordinates chr22: 23631722-23631762 and/or a 3′ breakpoint within chromosomal coordinates chr9: 133729484-133729524. In some embodiments, an ABL1 fusion polypeptide of the disclosure is a BCR-ABL1 fusion polypeptide encoded by a BCR-ABL1 fusion nucleic acid molecule comprising or resulting from a 5′ breakpoint within chromosomal coordinates chr22: 23633346-23634163 and/or a 3′ breakpoint within chromosomal coordinates chr9: 133609201-133609909. In some embodiments, an ABL1 fusion polypeptide of the disclosure is a BCR-ABL1 fusion polypeptide encoded by a BCR-ABL1 fusion nucleic acid molecule comprising or resulting from a 5′ breakpoint within chromosomal coordinates chr22: 23633432-23633796 and/or a 3′ breakpoint within chromosomal coordinates chr9: 133623903-133624461. In some embodiments, an ABL1 fusion polypeptide of the disclosure is a BCR-ABL1 fusion polypeptide encoded by a BCR-ABL1 fusion nucleic acid molecule comprising or resulting from a 5′ breakpoint within chromosomal coordinates chr22: 23633837-23634208 and/or a 3′ breakpoint within chromosomal coordinates chr9: 133665426-133665727. In some embodiments, an ABL1 fusion polypeptide of the disclosure is a BCR-ABL1 fusion polypeptide encoded by a BCR-ABL1 fusion nucleic acid molecule comprising or resulting from a 5′ breakpoint within chromosomal coordinates chr22: 23632858-23633068 and/or a 3′ breakpoint within chromosomal coordinates chr9: 133701595-133701846. In some embodiments, an ABL1 fusion polypeptide of the disclosure is a BCR-ABL1 fusion polypeptide encoded by a BCR-ABL1 fusion nucleic acid molecule comprising or resulting from a 5′ breakpoint within chromosomal coordinates chr22: 23633208-23633695 and/or a 3′ breakpoint within chromosomal coordinates chr9: 133702617-133702992. In some embodiments, an ABL1 fusion polypeptide of the disclosure is a NUP214-ABL1 fusion polypeptide encoded by a NUP214-ABL1 fusion nucleic acid molecule comprising or resulting from a 5′ breakpoint within chromosomal coordinates chr9: 134106039-134106079 and/or a 3′ breakpoint within chromosomal coordinates chr9: 133730222-133730262. In some embodiments, an ABL1 fusion polypeptide of the disclosure is an ABL1-AIF1L fusion polypeptide encoded by an ABL1-AIF1L fusion nucleic acid molecule comprising or resulting from a 5′ breakpoint within chromosomal coordinates chr9: 133755952-133755992 and/or a 3′ breakpoint within chromosomal coordinates chr9: 133989965-133990005. In some embodiments, an ABL1 fusion polypeptide of the disclosure is a RCSD1-ABL1 fusion polypeptide encoded by an RCSD1-ABL1 fusion nucleic acid molecule comprising or resulting from a 5′ breakpoint within chromosomal coordinates chr1: 167654668-167654708 and/or a 3′ breakpoint within chromosomal coordinates chr9: 133738173-133738213. In some embodiments, an ABL1 fusion polypeptide of the disclosure is an APOOL-ABL1 fusion polypeptide encoded by an APOOL-ABL1 fusion nucleic acid molecule comprising or resulting from a 5′ breakpoint within chromosomal coordinates chrX: 84302705-84302904 and/or a 3′ breakpoint within chromosomal coordinates chr9: 133729584-133729766. In some embodiments, an ABL1 fusion polypeptide of the disclosure is an ABL1-DNAH2 fusion polypeptide encoded by an ABL1-DNAH2 fusion nucleic acid molecule comprising or resulting from a 5′ breakpoint within chromosomal coordinates chr9: 133755953-133755993 and/or a 3′ breakpoint within chromosomal coordinates chr17: 7636399-7636439. In some embodiments, an ABL1 fusion polypeptide of the disclosure is a RHEX/C1orf186-ABL1 fusion polypeptide encoded by a RHEX/C1orf186-ABL1 fusion nucleic acid molecule comprising or resulting from a 5′ breakpoint within chromosomal coordinates chr1: 206288099-206288139 and/or a 3′ breakpoint within chromosomal coordinates chr9: 133729488-133729528. In some embodiments, an ABL1 fusion polypeptide of the disclosure is a RALGPS1-ABL1 fusion polypeptide encoded by a RALGPS1-ABL1 fusion nucleic acid molecule comprising or resulting from a 5′ breakpoint within chromosomal coordinates chr9: 129694023-129694282 and/or a 3′ breakpoint within chromosomal coordinates chr9: 133730118-133730529. In some embodiments, an ABL1 fusion polypeptide of the disclosure is a TSC1-ABL1 fusion polypeptide encoded by a TSC1-ABL1 fusion nucleic acid molecule comprising or resulting from a 5′ breakpoint within chromosomal coordinates chr9:135,776,998-135,777,038 and/or a 3′ breakpoint within chromosomal coordinates chr9: 133729491-133729531. In some embodiments, an ABL1 fusion polypeptide of the disclosure is a TSPAN18-ABL1 fusion polypeptide encoded by an TSPAN18-ABL1 fusion nucleic acid molecule comprising or resulting from a 5′ breakpoint within chromosomal coordinates chr11: 44881929-44881969 and/or a 3′ breakpoint within chromosomal coordinates chr9: 133729517-133729557. In some embodiments, an ABL1 fusion polypeptide of the disclosure is a ZNF804B-ABL1 fusion polypeptide encoded by a ZNF804B-ABL1 fusion nucleic acid molecule comprising or resulting from a 5′ breakpoint within chromosomal coordinates chr7: 88521265-88521397 and/or a 3′ breakpoint within chromosomal coordinates chr9: 133729414-133729519. In some embodiments, an ABL1 fusion polypeptide of the disclosure is a BCR-ABL1 fusion polypeptide encoded by a BCR-ABL1 fusion nucleic acid molecule comprising or resulting from a 5′ breakpoint within chromosomal coordinates chr22: 23631687-23632492 and/or a 3′ breakpoint within chromosomal coordinates chr9: 133604425-133604577. In some embodiments, an ABL1 fusion polypeptide of the disclosure is a BCR-ABL1 fusion polypeptide encoded by a BCR-ABL1 fusion nucleic acid molecule comprising or resulting from a 5′ breakpoint within chromosomal coordinates chr22: 23632288-23632656 and/or a 3′ breakpoint within chromosomal coordinates chr9: 133663291-133663682. In some embodiments, an ABL1 fusion polypeptide of the disclosure is a BCR-ABL1 fusion polypeptide encoded by a BCR-ABL1 fusion nucleic acid molecule comprising or resulting from a 5′ breakpoint within chromosomal coordinates chr22: 23634171-23634647 and/or a 3′ breakpoint within chromosomal coordinates chr9: 133638886-133639512. In some embodiments, an ABL1 fusion polypeptide of the disclosure is a BCR-ABL1 fusion polypeptide encoded by a BCR-ABL1 fusion nucleic acid molecule comprising or resulting from a 5′ breakpoint within chromosomal coordinates chr22: 23633910-23634678 and/or a 3′ breakpoint within chromosomal coordinates chr9: 133724778-133725351. In some embodiments, an ABL1 fusion polypeptide of the disclosure is an ABL1-NUP214 fusion polypeptide encoded by an ABL1-NUP214 fusion nucleic acid molecule comprising or resulting from a 5′ breakpoint within chromosomal coordinates chr9: 134090583-134090623 and/or a 3′ breakpoint within chromosomal coordinates chr9: 133710830-133710870. In some embodiments, an ABL1 fusion polypeptide of the disclosure is an AIF1L-ABL1 fusion polypeptide encoded by an AIF1L-ABL1 fusion nucleic acid molecule comprising or resulting from a 5′ breakpoint within chromosomal coordinates chr9: 133978032-133978137 and/or a 3′ breakpoint within chromosomal coordinates chr9: 133729409-133729618. In some embodiments, an ABL1 fusion polypeptide of the disclosure is a EXOSC2-ABL1 fusion polypeptide encoded by an EXOSC2-ABL1 fusion nucleic acid molecule comprising or resulting from a 5′ breakpoint within chromosomal coordinates chr9: 133579154-133579531 and/or a 3′ breakpoint within chromosomal coordinates chr9: 133589786-133589985. In some embodiments, an ABL1 fusion polypeptide of the disclosure is a NUP214-ABL1 fusion polypeptide encoded by a NUP214-ABL1 fusion nucleic acid molecule comprising or resulting from a 5′ breakpoint within chromosomal coordinates chr9: 134106080-134106120 and/or a 3′ breakpoint within chromosomal coordinates chr9: 133730195-133730235. In some embodiments, an ABL1 fusion polypeptide of the disclosure is a NUP214-ABL1 fusion polypeptide encoded by a NUP214-ABL1 fusion nucleic acid molecule comprising or resulting from a 5′ breakpoint within chromosomal coordinates chr9: 134106030-134106070 and/or a 3′ breakpoint within chromosomal coordinates chr9: 133730218-133730258. In some embodiments, an ABL1 fusion polypeptide of the disclosure is a NUP214-ABL1 fusion polypeptide encoded by a NUP214-ABL1 fusion nucleic acid molecule comprising or resulting from a 5′ breakpoint within chromosomal coordinates chr9: 134106046-134106086 and/or a 3′ breakpoint within chromosomal coordinates chr9: 133730203-133730243. In some embodiments, an ABL1 fusion polypeptide of the disclosure is a NUP214-ABL1 fusion polypeptide encoded by a NUP214-ABL1 fusion nucleic acid molecule comprising or resulting from a 5′ breakpoint within chromosomal coordinates chr9: 134106028-134106068 and/or a 3′ breakpoint within chromosomal coordinates chr9: 133730234-133730274. In some embodiments, an ABL1 fusion polypeptide of the disclosure is a BCR-ABL1 fusion polypeptide encoded by a BCR-ABL1 fusion nucleic acid molecule comprising or resulting from a 5′ breakpoint within chromosomal coordinates chr22: 23524272-23524312 and/or a 3′ breakpoint within chromosomal coordinates chr9: 133730218-133730258. In some embodiments, an ABL1 fusion polypeptide of the disclosure is a SNX2-ABL1 fusion polypeptide encoded by an SNX2-ABL1 fusion nucleic acid molecule comprising or resulting from a 5′ breakpoint within chromosomal coordinates chr5: 122135451-122135491 and/or a 3′ breakpoint within chromosomal coordinates chr9: 133738222-133738262. In some embodiments, an ABL1 fusion polypeptide of the disclosure is a SNX2-ABL1 fusion polypeptide encoded by an SNX2-ABL1 fusion nucleic acid molecule comprising or resulting from a 5′ breakpoint within chromosomal coordinates chr5: 122135445-122135485 and/or a 3′ breakpoint within chromosomal coordinates chr9: 133738214-133738254. In some embodiments, an ABL1 fusion polypeptide of the disclosure is a BCR-ABL1 fusion polypeptide encoded by a BCR-ABL1 fusion nucleic acid molecule comprising or resulting from a 5′ breakpoint within chromosomal coordinates chr22: 23631693-23632188 and/or a 3′ breakpoint within chromosomal coordinates chr9: 133607683-133608245. In some embodiments, an ABL1 fusion polypeptide of the disclosure is a BMP2K-ABL1 fusion polypeptide encoded by an BMP2K-ABL1 fusion nucleic acid molecule comprising or resulting from a 5′ breakpoint within chromosomal coordinates chr4: 79800022-79800062 and/or a 3′ breakpoint within chromosomal coordinates chr9: 133730205-133730245.

In some embodiments, an ABL1 fusion polypeptide of the disclosure is encoded by an ABL1 fusion nucleic acid molecule as listed in Table 2 in Example 1 herein, comprising or resulting from the corresponding exonic/intronic and/or chromosomal breakpoints as provided in Table 2 in Example 1 herein.

### Exons in Exemplary ABL1 Fusion Polypeptides

In some embodiments, an ABL1 fusion polypeptide of the disclosure is an EHMT1-ABL1 fusion polypeptide encoded by an ABL1 fusion nucleic acid molecule comprising a fusion between exon 4, or a portion thereof, of an EHMT1 gene and exon 2, or a portion thereof, of an ABL1 gene. In some embodiments, an ABL1 fusion polypeptide of the disclosure is a MED27-ABL1 fusion polypeptide encoded by an ABL1 fusion nucleic acid molecule comprising a fusion between exon 4, or a portion thereof, of a MED27 gene and exon 5, or a portion thereof, of an ABL1 gene. In some embodiments, an ABL1 fusion polypeptide of the disclosure is an RFFL-ABL1 fusion polypeptide encoded by an ABL1 fusion nucleic acid molecule comprising a fusion between exon 5, or a portion thereof, of an RFFL gene and exon 2, or a portion thereof, of an ABL1 gene. In some embodiments, an ABL1 fusion polypeptide of the disclosure is an SLC27A4-ABL1 fusion polypeptide encoded by an ABL1 fusion nucleic acid molecule comprising a fusion between exon 12, or a portion thereof, of an SLC27A4 gene and exon 6, or a portion thereof, of an ABL1 gene. In some embodiments, an ABL1 fusion polypeptide of the disclosure is a BCR-ABL1 fusion polypeptide encoded by an ABL1 fusion nucleic acid molecule comprising a fusion between exon 13, or a portion thereof, of a BCR gene and exon 2, or a portion thereof, of an ABL1 gene. In some embodiments, an ABL1 fusion polypeptide of the disclosure is a BCR-ABL1 fusion polypeptide encoded by an ABL1 fusion nucleic acid molecule comprising a fusion between exon 14, or a portion thereof, of a BCR gene and exon 2, or a portion thereof, of an ABL1 gene. In some embodiments, an ABL1 fusion polypeptide of the disclosure is a BCR-ABL1 fusion polypeptide encoded by an ABL1 fusion nucleic acid molecule comprising a fusion between exon 1, or a portion thereof, of a BCR gene and exon 3, or a portion thereof, of an ABL1 gene. In some embodiments, an ABL1 fusion polypeptide of the disclosure is a BCR-ABL1 fusion polypeptide encoded by an ABL1 fusion nucleic acid molecule comprising a fusion between exon 12, or a portion thereof, of a BCR gene and exon 2, or a portion thereof, of an ABL1 gene. In some embodiments, an ABL1 fusion polypeptide of the disclosure is a NUP214-ABL1 fusion polypeptide encoded by an ABL1 fusion nucleic acid molecule comprising a fusion between exon 34, or a portion thereof, of a NUP214 gene and exon 3, or a portion thereof, of an ABL1 gene. In some embodiments, an ABL1 fusion polypeptide of the disclosure is an ABL1-AIF1L fusion polypeptide encoded by an ABL1 fusion nucleic acid molecule comprising a fusion between exon 10, or a portion thereof, of an ABL1 gene and exon 4, or a portion thereof, of an AIF1L gene. In some embodiments, an ABL1 fusion polypeptide of the disclosure is an RCSD1-ABL1 fusion polypeptide encoded by an ABL1 fusion nucleic acid molecule comprising a fusion between exon 4, or a portion thereof, of an RCSD1gene and exon 4, or a portion thereof, of an ABL1 gene. In some embodiments, an ABL1 fusion polypeptide of the disclosure is an APOOL-ABL1 fusion polypeptide encoded by an ABL1 fusion nucleic acid molecule comprising a fusion between exon 2, or a portion thereof, of an APOOL gene and exon 2, or a portion thereof, of an ABL1 gene. In some embodiments, an ABL1 fusion polypeptide of the disclosure is an ABL1-DNAH2 fusion polypeptide encoded by an ABL1 fusion nucleic acid molecule comprising a fusion between exon 10, or a portion thereof, of an ABL1 gene and exon 4, or a portion thereof, of a DNAH2 gene. In some embodiments, an ABL1 fusion polypeptide of the disclosure is a RHEX/C1orf186-ABL1 fusion polypeptide encoded by an ABL1 fusion nucleic acid molecule comprising a fusion between exon 1, or a portion thereof, of a RHEX/Clorf186 gene and exon 2, or a portion thereof, of an ABL1 gene. In some embodiments, an ABL1 fusion polypeptide of the disclosure is a RALGPS1-ABL1 fusion polypeptide encoded by an ABL1 fusion nucleic acid molecule comprising a fusion between exon 1, or a portion thereof, of a RALGPS1 gene and exon 3, or a portion thereof, of an ABL1 gene. In some embodiments, an ABL1 fusion polypeptide of the disclosure is a TSC1-ABL1 fusion polypeptide encoded by an ABL1 fusion nucleic acid molecule comprising a fusion between exon 19, or a portion thereof, of a TSC1 gene and exon 2, or a portion thereof, of an ABL1 gene. In some embodiments, an ABL1 fusion polypeptide of the disclosure is a TSPAN18-ABL1 fusion polypeptide encoded by an ABL1 fusion nucleic acid molecule comprising a fusion between exon 2, or a portion thereof, of a TSPAN18 gene and exon 2, or a portion thereof, of an ABL1 gene. In some embodiments, an ABL1 fusion polypeptide of the disclosure is a ZNF804B-ABL1 fusion polypeptide encoded by an ABL1 fusion nucleic acid molecule comprising a fusion between exon 1, or a portion thereof, of a ZNF804B gene and exon 2, or a portion thereof, of an ABL1 gene. In some embodiments, an ABL1 fusion polypeptide of the disclosure is an ABL1-NUP214 fusion polypeptide encoded by an ABL1 fusion nucleic acid molecule comprising a fusion between exon 1, or a portion thereof, of an ABL1 gene and exon 31, or a portion thereof, of a NUP214 gene. In some embodiments, an ABL1 fusion polypeptide of the disclosure is an AIF1L-ABL1 fusion polypeptide encoded by an ABL1 fusion nucleic acid molecule comprising a fusion between exon 2, or a portion thereof, of an AIF1L gene and exon 2, or a portion thereof, of an ABL1 gene. In some embodiments, an ABL1 fusion polypeptide of the disclosure is an EXOSC2-ABL1 fusion polypeptide encoded by an ABL1 fusion nucleic acid molecule comprising a fusion between exon 9, or a portion thereof, of an EXOSC2 gene and exon 1, or a portion thereof, of an ABL1 gene. In some embodiments, an ABL1 fusion polypeptide of the disclosure is an SNX2-ABL1 fusion polypeptide encoded by an ABL1 fusion nucleic acid molecule comprising a fusion between exon 3, or a portion thereof, of an SNX2 gene and exon 4, or a portion thereof, of an ABL1 gene. In some embodiments, an ABL1 fusion polypeptide of the disclosure is a BMP2K-ABL1 fusion polypeptide encoded by an ABL1 fusion nucleic acid molecule comprising a fusion between exon 15, or a portion thereof, of a BMP2K gene and exon 3, or a portion thereof, of an ABL1 gene.

In some embodiments, an ABL1 fusion polypeptide of the disclosure is an EHMT1-ABL1 fusion polypeptide, comprising a fusion between an amino acid sequence encoded by exon 4, or a portion thereof, of an EHMT1 gene and an amino acid sequence encoded by exon 2, or a portion thereof, of an ABL1 gene. In some embodiments, an ABL1 fusion polypeptide of the disclosure is a MED27-ABL1 fusion polypeptide, comprising a fusion between an amino acid sequence encoded by exon 4, or a portion thereof, of a MED27 gene and an amino acid sequence encoded by exon 5, or a portion thereof, of an ABL1 gene. In some embodiments, an ABL1 fusion polypeptide of the disclosure is an RFFL-ABL1 fusion polypeptide, comprising a fusion between an amino acid sequence encoded by exon 5, or a portion thereof, of an RFFL gene and an amino acid sequence encoded by exon 2, or a portion thereof, of an ABL1 gene. In some embodiments, an ABL1 fusion polypeptide of the disclosure is an SLC27A4-ABL1 fusion polypeptide, comprising a fusion between an amino acid sequence encoded by exon 12, or a portion thereof, of an SLC27A4 gene and an amino acid sequence encoded by exon 6, or a portion thereof, of an ABL1 gene. In some embodiments, an ABL1 fusion polypeptide of the disclosure is a BCR-ABL1 fusion polypeptide, comprising a fusion between an amino acid sequence encoded by exon 13, or a portion thereof, of a BCR gene and an amino acid sequence encoded by exon 2, or a portion thereof, of an ABL1 gene. In some embodiments, an ABL1 fusion polypeptide of the disclosure is a BCR-ABL1 fusion polypeptide, comprising a fusion between an amino acid sequence encoded by exon 14, or a portion thereof, of a BCR gene and an amino acid sequence encoded by exon 2, or a portion thereof, of an ABL1 gene. In some embodiments, an ABL1 fusion polypeptide of the disclosure is a BCR-ABL1 fusion polypeptide, comprising a fusion between an amino acid sequence encoded by exon 1, or a portion thereof, of a BCR gene and an amino acid sequence encoded by exon 3, or a portion thereof, of an ABL1 gene. In some embodiments, an ABL1 fusion polypeptide of the disclosure is a BCR-ABL1 fusion polypeptide, comprising a fusion between an amino acid sequence encoded by exon 12, or a portion thereof, of a BCR gene and an amino acid sequence encoded by exon 2, or a portion thereof, of an ABL1 gene. In some embodiments, an ABL1 fusion polypeptide of the disclosure is a NUP214-ABL1 fusion polypeptide, comprising a fusion between an amino acid sequence encoded by exon 34, or a portion thereof, of a NUP214 gene and an amino acid sequence encoded by exon 3, or a portion thereof, of an ABL1 gene. In some embodiments, an ABL1 fusion polypeptide of the disclosure is an ABL1-AIF1L fusion polypeptide, comprising a fusion between an amino acid sequence encoded by exon 10, or a portion thereof, of an ABL1 gene and an amino acid sequence encoded by exon 4, or a portion thereof, of an AIF1L gene. In some embodiments, an ABL1 fusion polypeptide of the disclosure is an RCSD1-ABL1 fusion polypeptide, comprising a fusion between an amino acid sequence encoded by exon 4, or a portion thereof, of an RCSD1gene and an amino acid sequence encoded by exon 4, or a portion thereof, of an ABL1 gene. In some embodiments, an ABL1 fusion polypeptide of the disclosure is an APOOL-ABL1 fusion polypeptide, comprising a fusion between an amino acid sequence encoded by exon 2, or a portion thereof, of an APOOL gene and an amino acid sequence encoded by exon 2, or a portion thereof, of an ABL1 gene. In some embodiments, an ABL1 fusion polypeptide of the disclosure is an ABL1-DNAH2 fusion polypeptide, comprising a fusion between an amino acid sequence encoded by exon 10, or a portion thereof, of an ABL1 gene and an amino acid sequence encoded by exon 4, or a portion thereof, of a DNAH2 gene. In some embodiments, an ABL1 fusion polypeptide of the disclosure is a RHEX/C1orf186-ABL1 fusion polypeptide, comprising a fusion between an amino acid sequence encoded by exon 1, or a portion thereof, of a RHEX/Clorf186 gene and an amino acid sequence encoded by exon 2, or a portion thereof, of an ABL1 gene. In some embodiments, an ABL1 fusion polypeptide of the disclosure is a RALGPS1-ABL1 fusion polypeptide, comprising a fusion between an amino acid sequence encoded by exon 1, or a portion thereof, of a RALGPS1 gene and an amino acid sequence encoded by exon 3, or a portion thereof, of an ABL1 gene. In some embodiments, an ABL1 fusion polypeptide of the disclosure is a TSC1-ABL1 fusion polypeptide, comprising a fusion between an amino acid sequence encoded by exon 19, or a portion thereof, of a TSC1 gene and an amino acid sequence encoded by exon 2, or a portion thereof, of an ABL1 gene. In some embodiments, an ABL1 fusion polypeptide of the disclosure is a TSPAN18-ABL1 fusion polypeptide, comprising a fusion between an amino acid sequence encoded by exon 2, or a portion thereof, of a TSPAN18 gene and an amino acid sequence encoded by exon 2, or a portion thereof, of an ABL1 gene. In some embodiments, an ABL1 fusion polypeptide of the disclosure is a ZNF804B-ABL1 fusion polypeptide, comprising a fusion between an amino acid sequence encoded by exon 1, or a portion thereof, of a ZNF804B gene and an amino acid sequence encoded by exon 2, or a portion thereof, of an ABL1 gene. In some embodiments, an ABL1 fusion polypeptide of the disclosure is an ABL1-NUP214 fusion polypeptide, comprising a fusion between an amino acid sequence encoded by exon 1, or a portion thereof, of an ABL1 gene and an amino acid sequence encoded by exon 31, or a portion thereof, of a NUP214 gene. In some embodiments, an ABL1 fusion polypeptide of the disclosure is an AIF1L-ABL1 fusion polypeptide, comprising a fusion between an amino acid sequence encoded by exon 2, or a portion thereof, of an AIF1L gene and an amino acid sequence encoded by exon 2, or a portion thereof, of an ABL1 gene. In some embodiments, an ABL1 fusion polypeptide of the disclosure is an EXOSC2-ABL1 fusion polypeptide, comprising a fusion between an amino acid sequence encoded by exon 9, or a portion thereof, of an EXOSC2 gene and an amino acid sequence encoded by exon 1, or a portion thereof, of an ABL1 gene. In some embodiments, an ABL1 fusion polypeptide of the disclosure is an SNX2-ABL1 fusion polypeptide, comprising a fusion between an amino acid sequence encoded by exon 3, or a portion thereof, of an SNX2 gene and an amino acid sequence encoded by exon 4, or a portion thereof, of an ABL1 gene. In some embodiments, an ABL1 fusion polypeptide of the disclosure is a BMP2K-ABL1 fusion polypeptide, comprising a fusion between an amino acid sequence encoded by exon 15, or a portion thereof, of a BMP2K gene and an amino acid sequence encoded by exon 3, or a portion thereof, of an ABL1 gene.

In some embodiments, an ABL1 fusion polypeptide of the disclosure is an EHMT1-ABL1 fusion polypeptide encoded by an ABL1 fusion nucleic acid molecule comprising, in 5′ to 3′ direction, exons 1-3 and exon 4, or a portion thereof, of an EHMT1 gene, fused to exon 2, or a portion thereof, and exons 3-11 of an ABL1 gene. In some embodiments, an ABL1 fusion polypeptide of the disclosure is a MED27-ABL1 fusion polypeptide encoded by an ABL1 fusion nucleic acid molecule comprising, in 5′ to 3′ direction, exons 1-3 and exon 4, or a portion thereof, of a MED27 gene, fused to exon 5, or a portion thereof, and exons 6-11 of an ABL1 gene. In some embodiments, an ABL1 fusion polypeptide of the disclosure is an RFFL-ABL1 fusion polypeptide encoded by an ABL1 fusion nucleic acid molecule comprising, in 5′ to 3′ direction, exons 1-4 and exon 5, or a portion thereof, of an RFFL gene, fused to exon 2, or a portion thereof, and exons 3-11 of an ABL1 gene. In some embodiments, an ABL1 fusion polypeptide of the disclosure is a SLC27A4-ABL1 fusion polypeptide encoded by an ABL1 fusion nucleic acid molecule comprising, in 5′ to 3′ direction, exons 1-11 and exon 12, or a portion thereof, of an SLC27A4 gene, fused to exon 6, or a portion thereof, and exons 7-11 of an ABL1 gene. In some embodiments, an ABL1 fusion polypeptide of the disclosure is a BCR-ABL1 fusion polypeptide encoded by an ABL1 fusion nucleic acid molecule comprising, in 5′ to 3′ direction, exons 1-12 and exon 13, or a portion thereof, of a BCR gene, fused to exon 2, or a portion thereof, and exons 3-11 of an ABL1 gene. In some embodiments, an ABL1 fusion polypeptide of the disclosure is a BCR-ABL1 fusion polypeptide encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene. In some embodiments, an ABL1 fusion polypeptide of the disclosure is a NUP214-ABL1 fusion polypeptide encoded by an ABL1 fusion nucleic acid molecule comprising, in 5′ to 3′ direction, exons 1-33 and exon 34, or a portion thereof, of a NUP214 gene, fused to exon 3, or a portion thereof, and exons 4-11 of an ABL1 gene. In some embodiments, an ABL1 fusion polypeptide of the disclosure is an ABL1-AIF1L fusion polypeptide encoded by an ABL1 fusion nucleic acid molecule comprising, in 5′ to 3′ direction, exons 1-9 and exon 10, or a portion thereof, of an ABL1 gene, fused to exon 4, or a portion thereof, and exons 5-6 of an AIF1L gene. In some embodiments, an ABL1 fusion polypeptide of the disclosure is a RCSD1-ABL1 fusion polypeptide encoded by an ABL1 fusion nucleic acid molecule comprising, in 5′ to 3′ direction, exons 1-3 and exon 4, or a portion thereof, of an RCSD1 gene, fused to exon 4, or a portion thereof, and exons 5-11 of an ABL1 gene. In some embodiments, an ABL1 fusion polypeptide of the disclosure is an APOOL-ABL1 fusion polypeptide encoded by an ABL1 fusion nucleic acid molecule comprising, in 5′ to 3′ direction, exon 1 and exon 2, or a portion thereof, of an APOOL gene, fused to exon 2, or a portion thereof, and exons 3-11 of an ABL1 gene. In some embodiments, an ABL1 fusion polypeptide of the disclosure is an ABL1-DNAH2 fusion polypeptide encoded by an ABL1 fusion nucleic acid molecule comprising, in 5′ to 3′ direction, exons 1-9 and exon 10, or a portion thereof, of an ABL1 gene, fused to exon 4, or a portion thereof, and exons 5-85 of a DNAH2 gene. In some embodiments, an ABL1 fusion polypeptide of the disclosure is a RHEX/C1orf186-ABL1 fusion polypeptide encoded by an ABL1 fusion nucleic acid molecule comprising, in 5′ to 3′ direction, exon 1, or a portion thereof, of a RHEX/Clorf186 gene, fused to exon 2, or a portion thereof, and exons 3-11 of an ABL1 gene. In some embodiments, an ABL1 fusion polypeptide of the disclosure is a RALGPS1-ABL1 fusion polypeptide encoded by an ABL1 fusion nucleic acid molecule comprising, in 5′ to 3′ direction, exon 1, or a portion thereof, of a RALGPS1 gene, fused to exon 3, or a portion thereof, and exons 4-11 of an ABL1 gene. In some embodiments, an ABL1 fusion polypeptide of the disclosure is a TSC1-ABL1 fusion polypeptide encoded by an ABL1 fusion nucleic acid molecule comprising, in the 5′ to 3′ direction, exons 1-18 and exon 19, or a portion thereof, of a TSC1 gene, fused to exon 2, or a portion thereof, and exons 3-11 of an ABL1 gene. In some embodiments, an ABL1 fusion polypeptide of the disclosure is a TSPAN18-ABL1 fusion polypeptide encoded by an ABL1 fusion nucleic acid molecule comprising, in 5′ to 3′ direction, exon 1 and exon 2, or a portion thereof, of a TSPAN18 gene, fused to exon 2, or a portion thereof, and exons 3-11 of an ABL1 gene. In some embodiments, an ABL1 fusion polypeptide of the disclosure is a ZNF804B-ABL1 fusion polypeptide encoded by an ABL1 fusion nucleic acid molecule comprising, in 5′ to 3′ direction, exon 1, or a portion thereof, of a ZNF804B gene, fused to exon 2, or a portion thereof, and exons 3-11 of an ABL1 gene. In some embodiments, an ABL1 fusion polypeptide of the disclosure is an ABL1-NUP214 fusion polypeptide encoded by an ABL1 fusion nucleic acid molecule comprising, in 5′ to 3′ direction, exon 1, or a portion thereof, of an ABL1 gene, fused to exon 31, or a portion thereof, and exons 32-36 of a NUP214 gene. In some embodiments, an ABL1 fusion polypeptide of the disclosure is an AIF1L-ABL1 fusion polypeptide encoded by an ABL1 fusion nucleic acid molecule comprising, in 5′ to 3′ direction, exon 1 and exon 2, or a portion thereof, of an AIF1L gene, fused to exon 2, or a portion thereof, and exons 3-11 of an ABL1 gene. In some embodiments, an ABL1 fusion polypeptide of the disclosure is an EXOSC2-ABL1 fusion polypeptide encoded by an ABL1 fusion nucleic acid molecule comprising, in 5′ to 3′ direction, exons 1-8 and exon 9, or a portion thereof, of an EXOSC2 gene, fused to exon 1, or a portion thereof, and exons 2-11 of an ABL1 gene. In some embodiments, an ABL1 fusion polypeptide of the disclosure is a BCR-ABL1 fusion polypeptide encoded by an ABL1 fusion nucleic acid molecule comprising, in 5′ to 3′ direction, exon 1, or a portion thereof, of a BCR gene, fused to exon 3, or a portion thereof, and exons 4-11 of an ABL1 gene. In some embodiments, an ABL1 fusion polypeptide of the disclosure is a SNX2-ABL1 fusion polypeptide encoded by an ABL1 fusion nucleic acid molecule comprising, in 5′ to 3′ direction, exons 1-2 and exon 3, or a portion thereof, of an SNX2 gene, fused to exon 4, or a portion thereof, and exons 5-11 of an ABL1 gene. In some embodiments, an ABL1 fusion polypeptide of the disclosure is a BCR-ABL1 fusion polypeptide encoded by an ABL1 fusion nucleic acid molecule comprising, in 5′ to 3′ direction, exons 1-11 and exon 12, or a portion thereof, of a BCR gene, fused to exon 2, or a portion thereof, and exons 3-11 of an ABL1 gene. In some embodiments, an ABL1 fusion polypeptide of the disclosure is a BMP2K-ABL1 fusion polypeptide encoded by an ABL1 fusion nucleic acid molecule comprising, in 5′ to 3′ direction, exons 1-14 and exon 15, or a portion thereof, of a BMP2K gene, fused to exon 3, or a portion thereof, and exons 4-11 of an ABL1 gene.

In some embodiments, an ABL1 fusion polypeptide of the disclosure is an EHMT1-ABL1 fusion polypeptide comprising, in the N- to C-terminus direction, an amino acid sequence encoded by exons 1-3 and exon 4, or a portion thereof, of an EHMT1 gene, fused to an amino acid sequence encoded by exon 2, or a portion thereof, and exons 3-11 of an ABL1 gene. In some embodiments, an ABL1 fusion polypeptide of the disclosure is a MED27-ABL1 fusion polypeptide comprising, in the N- to C-terminus direction, an amino acid sequence encoded by exons 1-3 and exon 4, or a portion thereof, of a MED27 gene, fused to an amino acid sequence encoded by exon 5, or a portion thereof, and exons 6-11 of an ABL1 gene. In some embodiments, an ABL1 fusion polypeptide of the disclosure is an RFFL-ABL1 fusion polypeptide comprising, in the N- to C-terminus direction, an amino acid sequence encoded by exons 1-4 and exon 5, or a portion thereof, of an RFFL gene, fused to an amino acid sequence encoded by exon 2, or a portion thereof, and exons 3-11 of an ABL1 gene. In some embodiments, an ABL1 fusion polypeptide of the disclosure is a SLC27A4-ABL1 fusion polypeptide comprising, in the N- to C-terminus direction, an amino acid sequence encoded by exons 1-11 and exon 12, or a portion thereof, of an SLC27A4 gene, fused to an amino acid sequence encoded by exon 6, or a portion thereof, and exons 7-11 of an ABL1 gene. In some embodiments, an ABL1 fusion polypeptide of the disclosure is a BCR-ABL1 fusion polypeptide comprising, in the N- to C-terminus direction, an amino acid sequence encoded by exons 1-12 and exon 13, or a portion thereof, of a BCR gene, fused to an amino acid sequence encoded by exon 2, or a portion thereof, and exons 3-11 of an ABL1 gene. In some embodiments, an ABL1 fusion polypeptide of the disclosure is a BCR-ABL1 fusion polypeptide comprising, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene. In some embodiments, an ABL1 fusion polypeptide of the disclosure is a NUP214-ABL1 fusion polypeptide comprising, in the N- to C-terminus direction, an amino acid sequence encoded by exons 1-33 and exon 34, or a portion thereof, of a NUP214 gene, fused to an amino acid sequence encoded by exon 3, or a portion thereof, and exons 4-11 of an ABL1 gene. In some embodiments, an ABL1 fusion polypeptide of the disclosure is an ABL1-AIF1L fusion polypeptide comprising, in the N- to C-terminus direction, an amino acid sequence encoded by exons 1-9 and exon 10, or a portion thereof, of an ABL1 gene, fused to an amino acid sequence encoded by exon 4, or a portion thereof, and exons 5-6 of an AIF1L gene. In some embodiments, an ABL1 fusion polypeptide of the disclosure is a RCSD1-ABL1 fusion polypeptide comprising, in the N- to C-terminus direction, an amino acid sequence encoded by exons 1-3 and exon 4, or a portion thereof, of an RCSD1 gene, fused to an amino acid sequence encoded by exon 4, or a portion thereof, and exons 5-11 of an ABL1 gene. In some embodiments, an ABL1 fusion polypeptide of the disclosure is an APOOL-ABL1 fusion polypeptide comprising, in the N- to C-terminus direction, an amino acid sequence encoded by exon 1 and exon 2, or a portion thereof, of an APOOL gene, fused to an amino acid sequence encoded by exon 2, or a portion thereof, and exons 3-11 of an ABL1 gene. In some embodiments, an ABL1 fusion polypeptide of the disclosure is an ABL1-DNAH2 fusion polypeptide comprising, in the N- to C-terminus direction, an amino acid sequence encoded by exons 1-9 and exon 10, or a portion thereof, of an ABL1 gene, fused to an amino acid sequence encoded by exon 4, or a portion thereof, and exons 5-85 of a DNAH2 gene. In some embodiments, an ABL1 fusion polypeptide of the disclosure is a RHEX/C1orf186-ABL1 fusion polypeptide comprising, in the N- to C-terminus direction, an amino acid sequence encoded by exon 1, or a portion thereof, of a RHEX/Clorf186 gene, fused to an amino acid sequence encoded by exon 2, or a portion thereof, and exons 3-11 of an ABL1 gene. In some embodiments, an ABL1 fusion polypeptide of the disclosure is a RALGPS1-ABL1 fusion polypeptide comprising, in the N- to C-terminus direction, an amino acid sequence encoded by exon 1, or a portion thereof, of a RALGPS1 gene, fused to an amino acid sequence encoded by exon 3, or a portion thereof, and exons 4-11 of an ABL1 gene. In some embodiments, an ABL1 fusion polypeptide of the disclosure is a TSC1-ABL1 fusion polypeptide comprising, in the N- to C-terminus direction, an amino acid sequence encoded by exons 1-18 and exon 19, or a portion thereof, of a TSC1 gene, fused to an amino acid sequence encoded by exon 2, or a portion thereof, and exons 3-11 of an ABL1 gene. In some embodiments, an ABL1 fusion polypeptide of the disclosure is a TSPAN18-ABL1 fusion polypeptide comprising, in the N- to C-terminus direction, an amino acid sequence encoded by exon 1 and exon 2, or a portion thereof, of a TSPAN18 gene, fused to an amino acid sequence encoded by exon 2, or a portion thereof, and exons 3-11 of an ABL1 gene. In some embodiments, an ABL1 fusion polypeptide of the disclosure is a ZNF804B-ABL1 fusion polypeptide comprising, in the N- to C-terminus direction, an amino acid sequence encoded by exon 1, or a portion thereof, of a ZNF804B gene, fused to an amino acid sequence encoded by exon 2, or a portion thereof, and exons 3-11 of an ABL1 gene. In some embodiments, an ABL1 fusion polypeptide of the disclosure is an ABL1-NUP214 fusion polypeptide comprising, in the N- to C-terminus direction, an amino acid sequence encoded by exon 1, or a portion thereof, of an ABL1 gene, fused to an amino acid sequence encoded by exon 31, or a portion thereof, and exons 32-36 of a NUP214 gene. In some embodiments, an ABL1 fusion polypeptide of the disclosure is an AIF1L-ABL1 fusion polypeptide comprising, in the N- to C-terminus direction, an amino acid sequence encoded by exon 1 and exon 2, or a portion thereof, of an AIF1L gene, fused to an amino acid sequence encoded by exon 2, or a portion thereof, and exons 3-11 of an ABL1 gene. In some embodiments, an ABL1 fusion polypeptide of the disclosure is an EXOSC2-ABL1 fusion polypeptide comprising, in the N- to C-terminus direction, an amino acid sequence encoded by exons 1-8 and exon 9, or a portion thereof, of an EXOSC2 gene, fused to an amino acid sequence encoded by exon 1, or a portion thereof, and exons 2-11 of an ABL1 gene. In some embodiments, an ABL1 fusion polypeptide of the disclosure is a BCR-ABL1 fusion polypeptide comprising, in the N- to C-terminus direction, an amino acid sequence encoded by exon 1, or a portion thereof, of a BCR gene, fused to an amino acid sequence encoded by exon 3, or a portion thereof, and exons 4-11 of an ABL1 gene. In some embodiments, an ABL1 fusion polypeptide of the disclosure is a SNX2-ABL1 fusion polypeptide comprising, in the N- to C-terminus direction, an amino acid sequence encoded by exons 1-2 and exon 3, or a portion thereof, of an SNX2 gene, fused to an amino acid sequence encoded by exon 4, or a portion thereof, and exons 5-11 of an ABL1 gene. In some embodiments, an ABL1 fusion polypeptide of the disclosure is a BCR-ABL1 fusion polypeptide comprising, in the N- to C-terminus direction, an amino acid sequence encoded by exons 1-11 and exon 12, or a portion thereof, of a BCR gene, fused to an amino acid sequence encoded by exon 2, or a portion thereof, and exons 3-11 of an ABL1 gene. In some embodiments, an ABL1 fusion polypeptide of the disclosure is a BMP2K-ABL1 fusion polypeptide comprising, in the N- to C-terminus direction, an amino acid sequence encoded by exons 1-14 and exon 15, or a portion thereof, of a BMP2K gene, fused to an amino acid sequence encoded by exon 3, or a portion thereof, and exons 4-11 of an ABL1 gene.

In some embodiments, an ABL1 fusion polypeptide of the disclosure is encoded by an ABL1 fusion nucleic acid molecule as listed in Table 2 in Example 1 herein, e.g., comprising the corresponding exons (or portions thereof) as listed in Table 2 in Example 1 herein. In some embodiments, an ABL1 fusion polypeptide of the disclosure comprises the corresponding exons (or portions thereof) as listed in Table 2 in Example 1 herein.

### Sequences of Exemplary ABL1 Fusion Polypeptides

In some embodiments, an EHMT1-ABL1 fusion polypeptide of the disclosure is encoded by an EHMT1-ABL1 fusion nucleic acid molecule comprising the nucleotide sequence of SEQ ID NO: 2, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 2. In some embodiments, a MED27-ABL1 fusion polypeptide of the disclosure is encoded by a MED27-ABL1 fusion nucleic acid molecule comprising the nucleotide sequence of SEQ ID NO: 3, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 3. In some embodiments, a RFFL-ABL1 fusion polypeptide of the disclosure is encoded by a RFFL-ABL1 fusion nucleic acid molecule comprising the nucleotide sequence of SEQ ID NO: 4, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 4. In some embodiments, a SLC27A4-ABL1 fusion polypeptide of the disclosure is encoded by a SLC27A4-ABL1 fusion nucleic acid molecule comprising the nucleotide sequence of SEQ ID NO: 5, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 5. In some embodiments, a BCR-ABL1 fusion polypeptide of the disclosure is encoded by a BCR-ABL1 fusion nucleic acid molecule comprising the nucleotide sequence of SEQ ID NO: 6, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 6. In some embodiments, a BCR-ABL1 fusion polypeptide of the disclosure is encoded by a BCR-ABL1 fusion nucleic acid molecule comprising the nucleotide sequence of SEQ ID NO: 7, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 7. In some embodiments, a BCR-ABL1 fusion polypeptide of the disclosure is encoded by a BCR-ABL1 fusion nucleic acid molecule comprising the nucleotide sequence of SEQ ID NO: 8, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 8. In some embodiments, a BCR-ABL1 fusion polypeptide of the disclosure is encoded by a BCR-ABL1 fusion nucleic acid molecule comprising the nucleotide sequence of SEQ ID NO: 9, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 9. In some embodiments, a BCR-ABL1 fusion polypeptide of the disclosure is encoded by a BCR-ABL1 fusion nucleic acid molecule comprising the nucleotide sequence of SEQ ID NO: 10, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 10. In some embodiments, a BCR-ABL1 fusion polypeptide of the disclosure is encoded by a BCR-ABL1 fusion nucleic acid molecule comprising the nucleotide sequence of SEQ ID NO: 11, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 11. In some embodiments, a BCR-ABL1 fusion polypeptide of the disclosure is encoded by a BCR-ABL1 fusion nucleic acid molecule comprising the nucleotide sequence of SEQ ID NO: 12, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 12. In some embodiments, a BCR-ABL1 fusion polypeptide of the disclosure is encoded by a BCR-ABL1 fusion nucleic acid molecule comprising the nucleotide sequence of SEQ ID NO: 13, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 13. In some embodiments, a BCR-ABL1 fusion polypeptide of the disclosure is encoded by a BCR-ABL1 fusion nucleic acid molecule comprising the nucleotide sequence of SEQ ID NO: 14, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 14. In some embodiments, a BCR-ABL1 fusion polypeptide of the disclosure is encoded by a BCR-ABL1 fusion nucleic acid molecule comprising the nucleotide sequence of SEQ ID NO: 15, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 15. In some embodiments, a BCR-ABL1 fusion polypeptide of the disclosure is encoded by a BCR-ABL1 fusion nucleic acid molecule comprising the nucleotide sequence of SEQ ID NO: 16, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 16. In some embodiments, a BCR-ABL1 fusion polypeptide of the disclosure is encoded by a BCR-ABL1 fusion nucleic acid molecule comprising the nucleotide sequence of SEQ ID NO: 17, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 17. In some embodiments, a BCR-ABL1 fusion polypeptide of the disclosure is encoded by a BCR-ABL1 fusion nucleic acid molecule comprising the nucleotide sequence of SEQ ID NO: 18, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 18. In some embodiments, a NUP214-ABL1 fusion polypeptide of the disclosure is encoded by a NUP214-ABL1 fusion nucleic acid molecule comprising the nucleotide sequence of SEQ ID NO: 19, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 19. In some embodiments, an ABL1-AIF1L fusion polypeptide of the disclosure is encoded by an ABL1-AIF1L fusion nucleic acid molecule comprising the nucleotide sequence of SEQ ID NO: 20, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 20. In some embodiments, a RCSD1-ABL1 fusion polypeptide of the disclosure is encoded by a RCSD1-ABL1 fusion nucleic acid molecule comprising the nucleotide sequence of SEQ ID NO: 21, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 21.

In some embodiments, an ABL1 fusion polypeptide of the disclosure is an EHMT1-ABL1 fusion polypeptide comprising the amino acid sequence of SEQ ID NO: 23, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 23.

(SEQ ID NO: 23)
MAAADAEAVPARGEPQQDCCVKTELLGEETPMAADEGSAEKQAGEAHMAADGETNGSCENSDASSHAN
AAKHTQDSARVNPQDGTNTLTRIAENGVSERDSEAAKQNHVTADDEVQTSVIGSNGYILNKPALQAQP
LRTTSTLASSLPGHAAKTLPGGAGKGRTPSAFPQTPAAPPATLGEGSADTEDRKLPAPGADVKVHRAR
KTMPKSVVGLHAASKDPREVREARDHKEPKEEINKNISDFGRQQLLPPFPSLHQSLPQNQCYMATTKS
QTEALQRPVASDFEPQGLSEAARWNSKENLLAGPSENDPNLFVALYDFVASGDNTLSITKGEKLRVLG
YNHNGEWCEAQTKNGQGWVPSNYITPVNSLEKHSWYHGPVSRNAAEYLLSSGINGSFLVRESESSPGQ
RSISLRYEGRVYHYRINTASDGKLYVSSESRFNTLAELVHHHSTVADGLITTLHYPAPKRNKPTVYGV
SPNYDKWEMERTDITMKHKLGGGQYGEVYEGVWKKYSLTVAVKTLKEDTMEVEEFLKEAAVMKEIKHP
NLVQLLGVCTREPPFYIITEFMTYGNLLDYLRECNRQEVNAVVLLYMATQISSAMEYLEKKNFIHRDL
AARNCLVGENHLVKVADFGLSRLMTGDTYTAHAGAKFPIKWTAPESLAYNKFSIKSDVWAFGVLLWEI
ATYGMSPYPGIDLSQVYELLEKDYRMERPEGCPEKVYELMRACWQWNPSDRPSFAEIHQAFETMFQES
SISDEVEKELGKQGVRGAVSTLLQAPELPTKTRTSRRAAEHRDTTDVPEMPHSKGQGESDPLDHEPAV
SPLLPRKERGPPEGGLNEDERLLPKDKKTNLFSALIKKKKKTAPTPPKRSSSFREMDGQPERRGAGEE
EGRDISNGALAFTPLDTADPAKSPKPSNGAGVPNGALRESGGSGFRSPHLWKKSSTLTSSRLATGEEE
GGGSSSKRFLRSCSASCVPHGAKDTEWRSVTLPRDLQSTGRQFDSSTFGGHKSEKPALPRKRAGENRS
DQVTRGTVTPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPPPPPAASAG
KAGGKPSQSPSQEAAGEAVLGAKTKATSLVDAVNSDAAKPSQPGEGLKKPVLPATPKPQSAKPSGTPI
SPAPVPSTLPSASSALAGDQPSSTAFIPLISTRVSLRKTRQPPERIASGAITKGVVLDSTEALCLAIS
RNSEQMASHSAVLEAGKNLYTFCVSYVDSIQQMRNKFAFREAINKLENNLRELQICPATAGSGPAATQ
DFSKLLSSVKEISDIVQR

In some embodiments, an ABL1 fusion polypeptide of the disclosure is a MED27-ABL1 fusion polypeptide comprising the amino acid sequence of SEQ ID NO: 24, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 24.

(SEQ ID NO: 24)
MADVINVSVNLEAFSQAISAIQALRSSVSRVFDCLKDGMRNKETLEGREKAFIAHFQDNLHSVNRDLN
ELERLSNLVGKPSENHPLHNSGLLSLDPVQDKTPLYSQLLQAYKWSNKLQYHAGLASGLLNQQSLKRS
ANQMGVSAKRRPKAQPTTLVLPPQYVDDVISRIDRMFPEMSIHLSRPNGTSAMLLEDTMEVEEFLKEA
AVMKEIKHPNLVQLLGVCTREPPFYIITEFMTYGNLLDYLRECNRQEVNAVVLLYMATQISSAMEYLE
KKNFIHRDLAARNCLVGENHLVKVADEGLSRLMTGDTYTAHAGAKFPIKWTAPESLAYNKFSIKSDVW
AFGVLLWEIATYGMSPYPGIDLSQVYELLEKDYRMERPEGCPEKVYELMRACWQWNPSDRPSFAEIHQ
AFETMFQESSISDEVEKELGKQGVRGAVSTLLQAPELPTKTRTSRRAAEHRDTTDVPEMPHSKGQGES
DPLDHEPAVSPLLPRKERGPPEGGLNEDERLLPKDKKTNLFSALIKKKKKTAPTPPKRSSSFREMDGQ
PERRGAGEEEGRDISNGALAFTPLDTADPAKSPKPSNGAGVPNGALRESGGSGFRSPHLWKKSSTLTS
SRLATGEEEGGGSSSKRFLRSCSASCVPHGAKDTEWRSVTLPRDLQSTGRQFDSSTEGGHKSEKPALP
RKRAGENRSDQVTRGTVTPPPRLVKKNEEAADEVFKDIMESSPGSSPPNLTPKPLRRQVTVAPASGLP
HKEEAGKGSALGTPAAAEPVTPTSKAGSGAPGGTSKGPAEESRVRRHKHSSESPGRDKGKLSRLKPAP
PPPPAASAGKAGGKPSQSPSQEAAGEAVLGAKTKATSLVDAVNSDAAKPSQPGEGLKKPVLPATPKPQ
SAKPSGTPISPAPVPSTLPSASSALAGDQPSSTAFIPLISTRVSLRKTRQPPERIASGAITKGVVLDS
TEALCLAISRNSEQMASHSAVLEAGKNLYTFCVSYVDSIQQMRNKFAFREAINKLENNLRELQICPAT
AGSGPAATQDFSKLLSSVKEISDIVQR

In some embodiments, an ABL1 fusion polypeptide of the disclosure is an RFFL-ABL1 fusion polypeptide comprising the amino acid sequence of SEQ ID NO: 25, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 25.

(SEQ ID NO: 25)
MWATCCNWFCLDGQPEEVPPPQGARMQAYSNPGYSSFPSPTGLEPSCKSCGAHFANTARKQTCLDCKK
NFCMTCSSQVGNGPRLCLLCQRFRATAFQREELMKMKVKDLRDYLSLHDISTEMCREKEELVLLVLGQ
QPVISQEDRTRASTLSPDFPEQQAFLTQPHSSMVPPTSPNLPSSSAQATSVPPAQVQENQQANGHVSQ
DQEEPVYLESVARVPAEDETQSIDSEDSFVPGRRASLSDLTDLEDIEGLTVRQLKEILARNFVNYKGC
CEKWELMERVTRLYKDQKGLQHLEALQRPVASDFEPQGLSEAARWNSKENLLAGPSENDPNLFVALYD
FVASGDNTLSITKGEKLRVLGYNHNGEWCEAQTKNGQGWVPSNYITPVNSLEKHSWYHGPVSRNAAEY
LLSSGINGSFLVRESESSPGQRSISLRYEGRVYHYRINTASDGKLYVSSESRFNTLAELVHHHSTVAD
GLITTLHYPAPKRNKPTVYGVSPNYDKWEMERTDITMKHKLGGGQYGEVYEGVWKKYSLTVAVKTLKE
DTMEVEEFLKEAAVMKEIKHPNLVQLLGVCTREPPFYIITEFMTYGNLLDYLRECNRQEVNAVVLLYM
ATQISSAMEYLEKKNFIHRDLAARNCLVGENHLVKVADFGLSRLMTGDTYTAHAGAKFPIKWTAPESL
AYNKFSIKSDVWAFGVLLWEIATYGMSPYPGIDLSQVYELLEKDYRMERPEGCPEKVYELMRACWQWN
PSDRPSFAEIHQAFETMFQESSISDEVEKELGKQGVRGAVSTLLQAPELPTKTRTSRRAAEHRDTTDV
PEMPHSKGQGESDPLDHEPAVSPLLPRKERGPPEGGLNEDERLLPKDKKTNLFSALIKKKKKTAPTPP
KRSSSFREMDGQPERRGAGEEEGRDISNGALAFTPLDTADPAKSPKPSNGAGVPNGALRESGGSGERS
PHLWKKSSTLTSSRLATGEEEGGGSSSKRFLRSCSASCVPHGAKDTEWRSVTLPRDLQSTGRQFDSST
FGGHKSEKPALPRKRAGENRSDQVTRGTVTPPPRLVKKNEEAADEVFKDIMESSPGSSPPNLTPKPLR
RQVTVAPASGLPHKEEAGKGSALGTPAAAEPVTPTSKAGSGAPGGTSKGPAEESRVRRHKHSSESPGR
DKGKLSRLKPAPPPPPAASAGKAGGKPSQSPSQEAAGEAVLGAKTKATSLVDAVNSDAAKPSQPGEGL
KKPVLPATPKPQSAKPSGTPISPAPVPSTLPSASSALAGDQPSSTAFIPLISTRVSLRKTRQPPERIA
SGAITKGVVLDSTEALCLAISRNSEQMASHSAVLEAGKNLYTFCVSYVDSIQQMRNKFAFREAINKLE
NNLRELQICPATAGSGPAATQDFSKLLSSVKEISDIVQR

In some embodiments, an ABL1 fusion polypeptide of the disclosure is a SLC27A4-ABL1 fusion polypeptide comprising the amino acid sequence of SEQ ID NO: 26, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 26.

(SEQ ID NO: 26)
MLLGASLVGVLLFSKLVLKLPWTQVGFSLLFLYLGSGGWRFIRVFIKTIRRDIFGGLVLLKVKAKVRQ
CLQERRTVPILFASTVRRHPDKTALIFEGTDTHWTFRQLDEYSSSVANFLQARGLASGDVAAIFMENR
NEFVGLWLGMAKLGVEAALINTNLRRDALLHCLTTSRARALVFGSEMASAICEVHASLDPSLSLFCSG
SWEPGAVPPSTEHLDPLLKDAPKHLPSCPDKGFTDKLFYIYTSGTTGLPKAAIVVHSRYYRMAALVYY
GFRMRPNDIVYDCLPLYHSAGNIVGIGQCLLHGMTVVIRKKFSASRFWDDCIKYNCTIVQYIGELCRY
LLNQPPREAENQHQVRMALGNGLRQSIWTNFSSRFHIPQVAEFYGATECNCSLGNFDSQVGACGFNSR
ILSFVYPIRLVRVNEDTMELIRGPDGVCIPCQPGEPGQLVGRIIQKDPLRRFDGYLNQGANNKKIAKD
VFKKGDQAYLTGDVLVMDELGYLYFRDRTGDTFRWKGENVSTTEVEGTLSRLLDMADVAVYGVEVPGT
EGRAGMAAVASPTGNCDLERFAQVLEKELPLYARPIFLRLLPELHKTGVCTREPPFYIITEEMTYGNL
LDYLRECNRQEVNAVVLLYMATQISSAMEYLEKKNFIHRDLAARNCLVGENHLVKVADFGLSRLMTGD
TYTAHAGAKFPIKWTAPESLAYNKFSIKSDVWAFGVLLWEIATYGMSPYPGIDLSQVYELLEKDYRME
RPEGCPEKVYELMRACWQWNPSDRPSFAEIHQAFETMFQESSISDEVEKELGKQGVRGAVSTLLQAPE
LPTKTRTSRRAAEHRDTTDVPEMPHSKGQGESDPLDHEPAVSPLLPRKERGPPEGGLNEDERLLPKDK
KTNLFSALIKKKKKTAPTPPKRSSSFREMDGQPERRGAGEEEGRDISNGALAFTPLDTADPAKSPKPS
NGAGVPNGALRESGGSGFRSPHLWKKSSTLTSSRLATGEEEGGGSSSKRFLRSCSASCVPHGAKDTEW
RSVTLPRDLQSTGRQFDSSTFGGHKSEKPALPRKRAGENRSDQVTRGTVTPPPRLVKKNEEAADEVFK
DIMESSPGSSPPNLTPKPLRRQVTVAPASGLPHKEEAGKGSALGTPAAAEPVTPTSKAGSGAPGGTSK
GPAEESRVRRHKHSSESPGRDKGKLSRLKPAPPPPPAASAGKAGGKPSQSPSQEAAGEAVLGAKTKAT
SLVDAVNSDAAKPSQPGEGLKKPVLPATPKPQSAKPSGTPISPAPVPSTLPSASSALAGDQPSSTAFI
PLISTRVSLRKTRQPPERIASGAITKGVVLDSTEALCLAISRNSEQMASHSAVLEAGKNLYTFCVSYV
DSIQQMRNKFAFREAINKLENNLRELQICPATAGSGPAATQDFSKLLSSVKEISDIVQR

In some embodiments, an ABL1 fusion polypeptide of the disclosure is a BCR-ABL1 fusion polypeptide comprising the amino acid sequence of SEQ ID NO: 27, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 27.

(SEQ ID NO: 27)
MVDPVGFAEAWKAQFPDSEPPRMELRSVGDIEQELERCKASIRRLEQEVNQERFRMIYLQTLLAKEKK
SYDRQRWGFRRAAQAPDGASEPRASASRPQPAPADGADPPPAEEPEARPDGEGSPGKARPGTARRPGA
AASGERDDRGPPASVAALRSNFERIRKGHGQPGADAEKPFYVNVEFHHERGLVKVNDKEVSDRISSLG
SQAMQMERKKSQHGAGSSVGDASRPPYRGRSSESSCGVDGDYEDAELNPRFLKDNLIDANGGSRPPWP
PLEYQPYQSIYVGGMMEGEGKGPLLRSQSTSEQEKRLTWPRRSYSPRSFEDCGGGYTPDCSSNENLTS
SEEDFSSGQSSRVSPSPTTYRMFRDKSRSPSQNSQQSFDSSSPPTPQCHKRHRHCPVVVSEATIVGVR
KTGQIWPNDGEGAFHGDADGSFGTPPGYGCAADRAEEQRRHQDGLPYIDDSPSSSPHLSSKGRGSRDA
LVSGALESTKASELDLEKGLEMRKWVLSGILASEETYLSHLEALLLPMKPLKAAATTSQPVLTSQQIE
TIFFKVPELYEIHKEFYDGLFPRVQQWSHQQRVGDLFQKLASQLGVYRAFVDNYGVAMEMAEKCCQAN
AQFAEISENLRARSNKDAKDPTTKNSLETLLYKPVDRVTRSTLVLHDLLKHTPASHPDHPLLQDALRI
SQNFLSSINEEITPRRQSMTVKKGEHRQLLKDSFMVELVEGARKLRHVFLFTDLLLCTKLKKQSGGKT
QQYDCKWYIPLTDLSFQMVDELEAVPNIPLVPDEELDALKIKISQIKNDIQREKRANKGSKATERLKK
KLSEQESLLLLMSPSMAFRVHSRNGKSYTFLISSDYERAEWRENIREQQKKCFRSFSLTSVELQMLIN
SCVKLQTVHSIPLTINKEEALQRPVASDFEPQGLSEAARWNSKENLLAGPSENDPNLFVALYDFVASG
DNTLSITKGEKLRVLGYNHNGEWCEAQTKNGQGWVPSNYITPVNSLEKHSWYHGPVSRNAAEYLLSSG
INGSFLVRESESSPGQRSISLRYEGRVYHYRINTASDGKLYVSSESRFNTLAELVHHHSTVADGLITT
LHYPAPKRNKPTVYGVSPNYDKWEMERTDITMKHKLGGGQYGEVYEGVWKKYSLTVAVKTLKEDTMEV
EEFLKEAAVMKEIKHPNLVQLLGVCTREPPFYIITEFMTYGNLLDYLRECNRQEVNAVVLLYMATQIS
SAMEYLEKKNFIHRDLAARNCLVGENHLVKVADFGLSRLMTGDTYTAHAGAKFPIKWTAPESLAYNKF
SIKSDVWAFGVLLWEIATYGMSPYPGIDLSQVYELLEKDYRMERPEGCPEKVYELMRACWQWNPSDRP
SFAEIHQAFETMFQESSISDEVEKELGKQGVRGAVSTLLQAPELPTKTRTSRRAAEHRDTTDVPEMPH
SKGQGESDPLDHEPAVSPLLPRKERGPPEGGLNEDERLLPKDKKTNLESALIKKKKKTAPTPPKRSSS
FREMDGQPERRGAGEEEGRDISNGALAFTPLDTADPAKSPKPSNGAGVPNGALRESGGSGFRSPHLWK
KSSTLTSSRLATGEEEGGGSSSKRFLRSCSASCVPHGAKDTEWRSVTLPRDLQSTGRQFDSSTFGGHK
SEKPALPRKRAGENRSDQVTRGTVTPPPRLVKKNEEAADEVFKDIMESSPGSSPPNLTPKPLRRQVTV
APASGLPHKEEAGKGSALGTPAAAEPVTPTSKAGSGAPGGTSKGPAEESRVRRHKHSSESPGRDKGKL
SRLKPAPPPPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPERIASGAIT
KGVVLDSTEALCLAISRNSEQMASHSAVLEAGKNLYTFCVSYVDSIQQMRNKFAFREAINKLENNLRE
LQICPATAGSGPAATQDFSKLLSSVKEISDIVQR

In some embodiments, an ABL1 fusion polypeptide of the disclosure is a BCR-ABL1 fusion polypeptide comprising the amino acid sequence of SEQ ID NO: 28, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 28.

(SEQ ID NO: 28)
MVDPVGFAEAWKAQFPDSEPPRMELRSVGDIEQELERCKASIRRLEQEVNQERFRMIYLQTLLAKEKK
SYDRQRWGFRRAAQAPDGASEPRASASRPQPAPADGADPPPAEEPEARPDGEGSPGKARPGTARRPGA
AASGERDDRGPPASVAALRSNFERIRKGHGQPGADAEKPFYVNVEFHHERGLVKVNDKEVSDRISSLG
SQAMQMERKKSQHGAGSSVGDASRPPYRGRSSESSCGVDGDYEDAELNPRFLKDNLIDANGGSRPPWP
PLEYQPYQSIYVGGMMEGEGKGPLLRSQSTSEQEKRLTWPRRSYSPRSFEDCGGGYTPDCSSNENLTS
SEEDFSSGQSSRVSPSPTTYRMFRDKSRSPSQNSQQSFDSSSPPTPQCHKRHRHCPVVVSEATIVGVR
KTGQIWPNDGEGAFHGDADGSFGTPPGYGCAADRAEEQRRHQDGLPYIDDSPSSSPHLSSKGRGSRDA
LVSGALESTKASELDLEKGLEMRKWVLSGILASEETYLSHLEALLLPMKPLKAAATTSQPVLTSQQIE
TIFFKVPELYEIHKEFYDGLFPRVQQWSHQQRVGDLFQKLASQLGVYRAFVDNYGVAMEMAEKCCQAN
AQFAEISENLRARSNKDAKDPTTKNSLETLLYKPVDRVTRSTLVLHDLLKHTPASHPDHPLLQDALRI
SQNFLSSINEEITPRRQSMTVKKGEHRQLLKDSFMVELVEGARKLRHVFLFTDLLLCTKLKKQSGGKT
QQYDCKWYIPLTDLSFQMVDELEAVPNIPLVPDEELDALKIKISQIKNDIQREKRANKGSKATERLKK
KLSEQESLLLLMSPSMAFRVHSRNGKSYTFLISSDYERAEWRENIREQQKKCFRSFSLTSVELQMLTN
SCVKLQTVHSIPLTINKEDDESPGLYGFLNVIVHSATGFKQSSKALQRPVASDFEPQGLSEAARWNSK
ENLLAGPSENDPNLFVALYDFVASGDNTLSITKGEKLRVLGYNHNGEWCEAQTKNGQGWVPSNYITPV
NSLEKHSWYHGPVSRNAAEYLLSSGINGSFLVRESESSPGQRSISLRYEGRVYHYRINTASDGKLYVS
SESRFNTLAELVHHHSTVADGLITTLHYPAPKRNKPTVYGVSPNYDKWEMERTDITMKHKLGGGQYGE
VYEGVWKKYSLTVAVKTLKEDTMEVEEFLKEAAVMKEIKHPNLVQLLGVCTREPPFYIITEFMTYGNL
LDYLRECNRQEVNAVVLLYMATQISSAMEYLEKKNFIHRDLAARNCLVGENHLVKVADFGLSRLMTGD
TYTAHAGAKFPIKWTAPESLAYNKESIKSDVWAFGVLLWEIATYGMSPYPGIDLSQVYELLEKDYRME
RPEGCPEKVYELMRACWQWNPSDRPSFAEIHQAFETMFQESSISDEVEKELGKQGVRGAVSTLLQAPE
LPTKTRTSRRAAEHRDTTDVPEMPHSKGQGESDPLDHEPAVSPLLPRKERGPPEGGLNEDERLLPKDK
KTNLFSALIKKKKKTAPTPPKRSSSFREMDGQPERRGAGEEEGRDISNGALAFTPLDTADPAKSPKPS
NGAGVPNGALRESGGSGERSPHLWKKSSTLTSSRLATGEEEGGGSSSKRFLRSCSASCVPHGAKDTEW
RSVTLPRDLQSTGRQFDSSTFGGHKSEKPALPRKRAGENRSDQVTRGTVTPPPRLVKKNEEAADEVFK
DIMESSPGSSPPNLTPKPLRRQVTVAPASGLPHKEEAGKGSALGTPAAAEPVTPTSKAGSGAPGGTSK
GPAEESRVRRHKHSSESPGRDKGKLSRLKPAPPPPPAASAGKAGGKPSQSPSQEAAGEAVLGAKTKAT
SLVDAVNSDAAKPSQPGEGLKKPVLPATPKPQSAKPSGTPISPAPVPSTLPSASSALAGDQPSSTAFI
PLISTRVSLRKTRQPPERIASGAITKGVVLDSTEALCLAISRNSEQMASHSAVLEAGKNLYTFCVSYV
DSIQQMRNKFAFREAINKLENNLRELQICPATAGSGPAATQDFSKLLSSVKEISDIVQR

In some embodiments, an ABL1 fusion polypeptide of the disclosure is a BCR-ABL1 fusion polypeptide comprising the amino acid sequence of SEQ ID NO: 29, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 29.

(SEQ ID NO: 29)
MVDPVGFAEAWKAQFPDSEPPRMELRSVGDIEQELERCKASIRRLEQEVNQERFRMIYLQTLLAKEKK
SYDRQRWGFRRAAQAPDGASEPRASASRPQPAPADGADPPPAEEPEARPDGEGSPGKARPGTARRPGA
AASGERDDRGPPASVAALRSNFERIRKGHGQPGADAEKPFYVNVEFHHERGLVKVNDKEVSDRISSLG
SQAMQMERKKSQHGAGSSVGDASRPPYRGRSSESSCGVDGDYEDAELNPRFLKDNLIDANGGSRPPWP
PLEYQPYQSIYVGGMMEGEGKGPLLRSQSTSEQEKRLTWPRRSYSPRSFEDCGGGYTPDCSSNENLTS
SEEDFSSGQSSRVSPSPTTYRMFRDKSRSPSQNSQQSFDSSSPPTPQCHKRHRHCPVVVSEATIVGVR
KTGQIWPNDGEGAFHGDADGSFGTPPGYGCAADRAEEQRRHQDGLPYIDDSPSSSPHLSSKGRGSRDA
LVSGALESTKASELDLEKGLEMRKWVLSGILASEETYLSHLEALLLPMKPLKAAATTSQPVLTSQQIE
TIFFKVPELYEIHKEFYDGLFPRVQQWSHQQRVGDLFQKLASQLGVYRAFVDNYGVAMEMAEKCCQAN
AQFAEISENLRARSNKDAKDPTTKNSLETLLYKPVDRVTRSTLVLHDLLKHTPASHPDHPLLQDALRI
SQNFLSSINEEITPRRQSMTVKKGEHRQLLKDSFMVELVEGARKLRHVFLFTDLLLCTKLKKQSGGKT
QQYDCKWYIPLTDLSFQMVDELEAVPNIPLVPDEELDALKIKISQIKNDIQREKRANKGSKATERLKK
KLSEQESLLLLMSPSMAFRVHSRNGKSYTFLISSDYERAEWRENIREQQKKCFRSFSLTSVELQMLTN
SCVKLQTVHSIPLTINKEDDESPGLYGFLNVIVHSATGFKQSSKALQRPVASDFEPQGLSEAARWNSK
ENLLAGPSENDPNLFVALYDFVASGDNTLSITKGEKLRVLGYNHNGEWCEAQTKNGQGWVPSNYITPV
NSLEKHSWYHGPVSRNAAEYLLSSGINGSFLVRESESSPGQRSISLRYEGRVYHYRINTASDGKLYVS
SESRFNTLAELVHHHSTVADGLITTLHYPAPKRNKPTVYGVSPNYDKWEMERTDITMKHKLGGGQYGE
VYEGVWKKYSLTVAVKTLKEDTMEVEEFLKEAAVMKEIKHPNLVQLLGVCTREPPFYIITEEMTYGNL
LDYLRECNRQEVNAVVLLYMATQISSAMEYLEKKNFIHRDLAARNCLVGENHLVKVADFGLSRLMTGD
TYTAHAGAKFPIKWTAPESLAYNKESIKSDVWAFGVLLWEIATYGMSPYPGIDLSQVYELLEKDYRME
RPEGCPEKVYELMRACWQWNPSDRPSFAEIHQAFETMFQESSISDEVEKELGKQGVRGAVSTLLQAPE
LPTKTRTSRRAAEHRDTTDVPEMPHSKGQGESDPLDHEPAVSPLLPRKERGPPEGGLNEDERLLPKDK
KTNLFSALIKKKKKTAPTPPKRSSSFREMDGQPERRGAGEEEGRDISNGALAFTPLDTADPAKSPKPS
NGAGVPNGALRESGGSGERSPHLWKKSSTLTSSRLATGEEEGGGSSSKRELRSCSASCVPHGAKDTEW
RSVTLPRDLQSTGRQFDSSTFGGHKSEKPALPRKRAGENRSDQVTRGTVTPPPRLVKKNEEAADEVFK
DIMESSPGSSPPNLTPKPLRRQVTVAPASGLPHKEEAGKGSALGTPAAAEPVTPTSKAGSGAPGGTSK
GPAEESRVRRHKHSSESPGRDKGKLSRLKPAPPPPPAASAGKAGGKPSQSPSQEAAGEAVLGAKTKAT
SLVDAVNSDAAKPSQPGEGLKKPVLPATPKPQSAKPSGTPISPAPVPSTLPSASSALAGDQPSSTAFI
PLISTRVSLRKTRQPPERIASGAITKGVVLDSTEALCLAISRNSEQMASHSAVLEAGKNLYTFCVSYV
DSIQQMRNKFAFREAINKLENNLRELQICPATAGSGPAATQDFSKLLSSVKEISDIVQR

In some embodiments, an ABL1 fusion polypeptide of the disclosure is a BCR-ABL1 fusion polypeptide comprising the amino acid sequence of SEQ ID NO: 30, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 30.

(SEQ ID NO: 30)
MVDPVGFAEAWKAQFPDSEPPRMELRSVGDIEQELERCKASIRRLEQEVNQERFRMIYLQTLLAKEKK
SYDRQRWGERRAAQAPDGASEPRASASRPQPAPADGADPPPAEEPEARPDGEGSPGKARPGTARRPGA
AASGERDDRGPPASVAALRSNFERIRKGHGQPGADAEKPFYVNVEFHHERGLVKVNDKEVSDRISSLG
SQAMQMERKKSQHGAGSSVGDASRPPYRGRSSESSCGVDGDYEDAELNPRFLKDNLIDANGGSRPPWP
PLEYQPYQSIYVGGMMEGEGKGPLLRSQSTSEQEKRLTWPRRSYSPRSFEDCGGGYTPDCSSNENLTS
SEEDFSSGQSSRVSPSPTTYRMFRDKSRSPSQNSQQSFDSSSPPTPQCHKRHRHCPVVVSEATIVGVR
KTGQIWPNDGEGAFHGDADGSFGTPPGYGCAADRAEEQRRHQDGLPYIDDSPSSSPHLSSKGRGSRDA
LVSGALESTKASELDLEKGLEMRKWVLSGILASEETYLSHLEALLLPMKPLKAAATTSQPVLTSQQIE
TIFFKVPELYEIHKEFYDGLFPRVQQWSHQQRVGDLFQKLASQLGVYRAFVDNYGVAMEMAEKCCQAN
AQFAEISENLRARSNKDAKDPTTKNSLETLLYKPVDRVTRSTLVLHDLLKHTPASHPDHPLLQDALRI
SQNELSSINEEITPRRQSMTVKKGEHRQLLKDSFMVELVEGARKLRHVFLFTDLLLCTKLKKQSGGKT
QQYDCKWYIPLTDLSFQMVDELEAVPNIPLVPDEELDALKIKISQIKNDIQREKRANKGSKATERLKK
KLSEQESLLLLMSPSMAFRVHSRNGKSYTFLISSDYERAEWRENIREQQKKCFRSFSLTSVELQMLTN
SCVKLQTVHSIPLTINKEDDESPGLYGFLNVIVHSATGFKQSSKALQRPVASDFEPQGLSEAARWNSK
ENLLAGPSENDPNLFVALYDFVASGDNTLSITKGEKLRVLGYNHNGEWCEAQTKNGQGWVPSNYITPV
NSLEKHSWYHGPVSRNAAEYLLSSGINGSFLVRESESSPGQRSISLRYEGRVYHYRINTASDGKLYVS
SESRFNTLAELVHHHSTVADGLITTLHYPAPKRNKPTVYGVSPNYDKWEMERTDITMKHKLGGGQYGE
VYEGVWKKYSLTVAVKTLKEDTMEVEEFLKEAAVMKEIKHPNLVQLLGVCTREPPFYIITEFMTYGNL
LDYLRECNRQEVNAVVLLYMATQISSAMEYLEKKNFIHRDLAARNCLVGENHLVKVADFGLSRLMTGD
TYTAHAGAKFPIKWTAPESLAYNKESIKSDVWAFGVLLWEIATYGMSPYPGIDLSQVYELLEKDYRME
RPEGCPEKVYELMRACWQWNPSDRPSFAEIHQAFETMFQESSISDEVEKELGKQGVRGAVSTLLQAPE
LPTKTRTSRRAAEHRDTTDVPEMPHSKGQGESDPLDHEPAVSPLLPRKERGPPEGGLNEDERLLPKDK
KTNLFSALIKKKKKTAPTPPKRSSSFREMDGQPERRGAGEEEGRDISNGALAFTPLDTADPAKSPKPS
NGAGVPNGALRESGGSGERSPHLWKKSSTLTSSRLATGEEEGGGSSSKRELRSCSASCVPHGAKDTEW
RSVTLPRDLQSTGRQFDSSTFGGHKSEKPALPRKRAGENRSDQVTRGTVTPPPRLVKKNEEAADEVFK
DIMESSPGSSPPNLTPKPLRRQVTVAPASGLPHKEEAGKGSALGTPAAAEPVTPTSKAGSGAPGGTSK
GPAEESRVRRHKHSSESPGRDKGKLSRLKPAPPPPPAASAGKAGGKPSQSPSQEAAGEAVLGAKTKAT
SLVDAVNSDAAKPSQPGEGLKKPVLPATPKPQSAKPSGTPISPAPVPSTLPSASSALAGDQPSSTAFI
PLISTRVSLRKTRQPPERIASGAITKGVVLDSTEALCLAISRNSEQMASHSAVLEAGKNLYTFCVSYV
DSIQQMRNKFAFREAINKLENNLRELQICPATAGSGPAATQDFSKLLSSVKEISDIVQR

In some embodiments, an ABL1 fusion polypeptide of the disclosure is a BCR-ABL1 fusion polypeptide comprising the amino acid sequence of SEQ ID NO: 31, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 31.

(SEQ ID NO: 31)
MVDPVGFAEAWKAQFPDSEPPRMELRSVGDIEQELERCKASIRRLEQEVNQERFRMIYLQTLLAKEKK
SYDRQRWGFRRAAQAPDGASEPRASASRPQPAPADGADPPPAEEPEARPDGEGSPGKARPGTARRPGA
AASGERDDRGPPASVAALRSNFERIRKGHGQPGADAEKPFYVNVEFHHERGLVKVNDKEVSDRISSLG
SQAMQMERKKSQHGAGSSVGDASRPPYRGRSSESSCGVDGDYEDAELNPRFLKDNLIDANGGSRPPWP
PLEYQPYQSIYVGGMMEGEGKGPLLRSQSTSEQEKRLTWPRRSYSPRSFEDCGGGYTPDCSSNENLTS
SEEDFSSGQSSRVSPSPTTYRMFRDKSRSPSQNSQQSFDSSSPPTPQCHKRHRHCPVVVSEATIVGVR
KTGQIWPNDGEGAFHGDADGSFGTPPGYGCAADRAEEQRRHQDGLPYIDDSPSSSPHLSSKGRGSRDA
LVSGALESTKASELDLEKGLEMRKWVLSGILASEETYLSHLEALLLPMKPLKAAATTSQPVLTSQQIE
TIFFKVPELYEIHKEFYDGLFPRVQQWSHQQRVGDLFQKLASQLGVYRAFVDNYGVAMEMAEKCCQAN
AQFAEISENLRARSNKDAKDPTTKNSLETLLYKPVDRVTRSTLVLHDLLKHTPASHPDHPLLQDALRI
SQNFLSSINEEITPRRQSMTVKKGEHRQLLKDSFMVELVEGARKLRHVFLFTDLLLCTKLKKQSGGKT
QQYDCKWYIPLTDLSFQMVDELEAVPNIPLVPDEELDALKIKISQIKNDIQREKRANKGSKATERLKK
KLSEQESLLLLMSPSMAFRVHSRNGKSYTFLISSDYERAEWRENIREQQKKCFRSFSLTSVELQMLTN
SCVKLQTVHSIPLTINKEDDESPGLYGFLNVIVHSATGFKQSSKALQRPVASDFEPQGLSEAARWNSK
ENLLAGPSENDPNLFVALYDFVASGDNTLSITKGEKLRVLGYNHNGEWCEAQTKNGQGWVPSNYITPV
NSLEKHSWYHGPVSRNAAEYLLSSGINGSFLVRESESSPGQRSISLRYEGRVYHYRINTASDGKLYVS
SESRFNTLAELVHHHSTVADGLITTLHYPAPKRNKPTVYGVSPNYDKWEMERTDITMKHKLGGGQYGE
VYEGVWKKYSLTVAVKTLKEDTMEVEEFLKEAAVMKEIKHPNLVQLLGVCTREPPFYIITEFMTYGNL
LDYLRECNRQEVNAVVLLYMATQISSAMEYLEKKNFIHRDLAARNCLVGENHLVKVADFGLSRLMTGD
TYTAHAGAKFPIKWTAPESLAYNKESIKSDVWAFGVLLWEIATYGMSPYPGIDLSQVYELLEKDYRME
RPEGCPEKVYELMRACWQWNPSDRPSFAEIHQAFETMFQESSISDEVEKELGKQGVRGAVSTLLQAPE
LPTKTRTSRRAAEHRDTTDVPEMPHSKGQGESDPLDHEPAVSPLLPRKERGPPEGGLNEDERLLPKDK
KTNLESALIKKKKKTAPTPPKRSSSFREMDGQPERRGAGEEEGRDISNGALAFTPLDTADPAKSPKPS
NGAGVPNGALRESGGSGERSPHLWKKSSTLTSSRLATGEEEGGGSSSKRELRSCSASCVPHGAKDTEW
RSVTLPRDLQSTGRQFDSSTFGGHKSEKPALPRKRAGENRSDQVTRGTVTPPPRLVKKNEEAADEVFK
DIMESSPGSSPPNLTPKPLRRQVTVAPASGLPHKEEAGKGSALGTPAAAEPVTPTSKAGSGAPGGTSK
GPAEESRVRRHKHSSESPGRDKGKLSRLKPAPPPPPAASAGKAGGKPSQSPSQEAAGEAVLGAKTKAT
SLVDAVNSDAAKPSQPGEGLKKPVLPATPKPQSAKPSGTPISPAPVPSTLPSASSALAGDQPSSTAFI
PLISTRVSLRKTRQPPERIASGAITKGVVLDSTEALCLAISRNSEQMASHSAVLEAGKNLYTFCVSYV
DSIQQMRNKFAFREAINKLENNLRELQICPATAGSGPAATQDFSKLLSSVKEISDIVQR

In some embodiments, an ABL1 fusion polypeptide of the disclosure is a BCR-ABL1 fusion polypeptide comprising the amino acid sequence of SEQ ID NO: 32, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 32.

(SEQ ID NO: 32)
MVDPVGFAEAWKAQFPDSEPPRMELRSVGDIEQELERCKASIRRLEQEVNQERFRMIYLQTLLAKEKK
SYDRQRWGERRAAQAPDGASEPRASASRPQPAPADGADPPPAEEPEARPDGEGSPGKARPGTARRPGA
AASGERDDRGPPASVAALRSNFERIRKGHGQPGADAEKPFYVNVEFHHERGLVKVNDKEVSDRISSLG
SQAMQMERKKSQHGAGSSVGDASRPPYRGRSSESSCGVDGDYEDAELNPRFLKDNLIDANGGSRPPWP
PLEYQPYQSIYVGGMMEGEGKGPLLRSQSTSEQEKRLTWPRRSYSPRSFEDCGGGYTPDCSSNENLTS
SEEDFSSGQSSRVSPSPTTYRMFRDKSRSPSQNSQQSFDSSSPPTPQCHKRHRHCPVVVSEATIVGVR
KTGQIWPNDGEGAFHGDADGSFGTPPGYGCAADRAEEQRRHQDGLPYIDDSPSSSPHLSSKGRGSRDA
LVSGALESTKASELDLEKGLEMRKWVLSGILASEETYLSHLEALLLPMKPLKAAATTSQPVLTSQQIE
TIFFKVPELYEIHKEFYDGLFPRVQQWSHQQRVGDLFQKLASQLGVYRAFVDNYGVAMEMAEKCCQAN
AQFAEISENLRARSNKDAKDPTTKNSLETLLYKPVDRVTRSTLVLHDLLKHTPASHPDHPLLQDALRI
SQNFLSSINEEITPRRQSMTVKKGEHRQLLKDSFMVELVEGARKLRHVFLFTDLLLCTKLKKQSGGKT
QQYDCKWYIPLTDLSFQMVDELEAVPNIPLVPDEELDALKIKISQIKNDIQREKRANKGSKATERLKK
KLSEQESLLLLMSPSMAFRVHSRNGKSYTFLISSDYERAEWRENIREQQKKCFRSFSLTSVELQMLTN
SCVKLQTVHSIPLTINKEDDESPGLYGFLNVIVHSATGFKQSSKALQRPVASDFEPQGLSEAARWNSK
ENLLAGPSENDPNLFVALYDFVASGDNTLSITKGEKLRVLGYNHNGEWCEAQTKNGQGWVPSNYITPV
NSLEKHSWYHGPVSRNAAEYLLSSGINGSFLVRESESSPGQRSISLRYEGRVYHYRINTASDGKLYVS
SESRFNTLAELVHHHSTVADGLITTLHYPAPKRNKPTVYGVSPNYDKWEMERTDITMKHKLGGGQYGE
VYEGVWKKYSLTVAVKTLKEDTMEVEEFLKEAAVMKEIKHPNLVQLLGVCTREPPFYIITEFMTYGNL
LDYLRECNRQEVNAVVLLYMATQISSAMEYLEKKNFIHRDLAARNCLVGENHLVKVADFGLSRLMTGD
TYTAHAGAKFPIKWTAPESLAYNKESIKSDVWAFGVLLWEIATYGMSPYPGIDLSQVYELLEKDYRME
RPEGCPEKVYELMRACWQWNPSDRPSFAEIHQAFETMFQESSISDEVEKELGKQGVRGAVSTLLQAPE
LPTKTRTSRRAAEHRDTTDVPEMPHSKGQGESDPLDHEPAVSPLLPRKERGPPEGGLNEDERLLPKDK
KTNLFSALIKKKKKTAPTPPKRSSSFREMDGQPERRGAGEEEGRDISNGALAFTPLDTADPAKSPKPS
NGAGVPNGALRESGGSGERSPHLWKKSSTLTSSRLATGEEEGGGSSSKRFLRSCSASCVPHGAKDTEW
RSVTLPRDLQSTGRQFDSSTFGGHKSEKPALPRKRAGENRSDQVTRGTVTPPPRLVKKNEEAADEVFK
DIMESSPGSSPPNLTPKPLRRQVTVAPASGLPHKEEAGKGSALGTPAAAEPVTPTSKAGSGAPGGTSK
GPAEESRVRRHKHSSESPGRDKGKLSRLKPAPPPPPAASAGKAGGKPSQSPSQEAAGEAVLGAKTKAT
SLVDAVNSDAAKPSQPGEGLKKPVLPATPKPQSAKPSGTPISPAPVPSTLPSASSALAGDQPSSTAFI
PLISTRVSLRKTRQPPERIASGAITKGVVLDSTEALCLAISRNSEQMASHSAVLEAGKNLYTFCVSYV
DSIQQMRNKFAFREAINKLENNLRELQICPATAGSGPAATQDFSKLLSSVKEISDIVQR

In some embodiments, an ABL1 fusion polypeptide of the disclosure is a BCR-ABL1 fusion polypeptide comprising the amino acid sequence of SEQ ID NO: 33, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 33.

(SEQ ID NO: 33)
MVDPVGFAEAWKAQFPDSEPPRMELRSVGDIEQELERCKASIRRLEQEVNQERFRMIYLQTLLAKEKK
SYDRQRWGERRAAQAPDGASEPRASASRPQPAPADGADPPPAEEPEARPDGEGSPGKARPGTARRPGA
AASGERDDRGPPASVAALRSNFERIRKGHGQPGADAEKPFYVNVEFHHERGLVKVNDKEVSDRISSLG
SQAMQMERKKSQHGAGSSVGDASRPPYRGRSSESSCGVDGDYEDAELNPRFLKDNLIDANGGSRPPWP
PLEYQPYQSIYVGGMMEGEGKGPLLRSQSTSEQEKRLTWPRRSYSPRSFEDCGGGYTPDCSSNENLTS
SEEDFSSGQSSRVSPSPTTYRMFRDKSRSPSQNSQQSFDSSSPPTPQCHKRHRHCPVVVSEATIVGVR
KTGQIWPNDGEGAFHGDADGSFGTPPGYGCAADRAEEQRRHQDGLPYIDDSPSSSPHLSSKGRGSRDA
LVSGALESTKASELDLEKGLEMRKWVLSGILASEETYLSHLEALLLPMKPLKAAATTSQPVLTSQQIE
TIFFKVPELYEIHKEFYDGLFPRVQQWSHQQRVGDLFQKLASQLGVYRAFVDNYGVAMEMAEKCCQAN
AQFAEISENLRARSNKDAKDPTTKNSLETLLYKPVDRVTRSTLVLHDLLKHTPASHPDHPLLQDALRI
SQNFLSSINEEITPRRQSMTVKKGEHRQLLKDSEMVELVEGARKLRHVFLFTDLLLCTKLKKQSGGKT
QQYDCKWYIPLTDLSFQMVDELEAVPNIPLVPDEELDALKIKISQIKNDIQREKRANKGSKATERLKK
KLSEQESLLLLMSPSMAFRVHSRNGKSYTFLISSDYERAEWRENIREQQKKCFRSFSLTSVELQMLTN
SCVKLQTVHSIPLTINKEDDESPGLYGFLNVIVHSATGFKQSSKALQRPVASDFEPQGLSEAARWNSK
ENLLAGPSENDPNLFVALYDFVASGDNTLSITKGEKLRVLGYNHNGEWCEAQTKNGQGWVPSNYITPV
NSLEKHSWYHGPVSRNAAEYLLSSGINGSFLVRESESSPGQRSISLRYEGRVYHYRINTASDGKLYVS
SESRFNTLAELVHHHSTVADGLITTLHYPAPKRNKPTVYGVSPNYDKWEMERTDITMKHKLGGGQYGE
VYEGVWKKYSLTVAVKTLKEDTMEVEEFLKEAAVMKEIKHPNLVQLLGVCTREPPFYIITEEMTYGNL
LDYLRECNRQEVNAVVLLYMATQISSAMEYLEKKNFIHRDLAARNCLVGENHLVKVADFGLSRLMTGD
TYTAHAGAKFPIKWTAPESLAYNKESIKSDVWAFGVLLWEIATYGMSPYPGIDLSQVYELLEKDYRME
RPEGCPEKVYELMRACWQWNPSDRPSFAEIHQAFETMFQESSISDEVEKELGKQGVRGAVSTLLQAPE
LPTKTRTSRRAAEHRDTTDVPEMPHSKGQGESDPLDHEPAVSPLLPRKERGPPEGGLNEDERLLPKDK
KTNLFSALIKKKKKTAPTPPKRSSSFREMDGQPERRGAGEEEGRDISNGALAFTPLDTADPAKSPKPS
NGAGVPNGALRESGGSGERSPHLWKKSSTLTSSRLATGEEEGGGSSSKRELRSCSASCVPHGAKDTEW
RSVTLPRDLQSTGRQFDSSTFGGHKSEKPALPRKRAGENRSDQVTRGTVTPPPRLVKKNEEAADEVFK
DIMESSPGSSPPNLTPKPLRRQVTVAPASGLPHKEEAGKGSALGTPAAAEPVTPTSKAGSGAPGGTSK
GPAEESRVRRHKHSSESPGRDKGKLSRLKPAPPPPPAASAGKAGGKPSQSPSQEAAGEAVLGAKTKAT
SLVDAVNSDAAKPSQPGEGLKKPVLPATPKPQSAKPSGTPISPAPVPSTLPSASSALAGDQPSSTAFI
PLISTRVSLRKTRQPPERIASGAITKGVVLDSTEALCLAISRNSEQMASHSAVLEAGKNLYTFCVSYV
DSIQQMRNKFAFREAINKLENNLRELQICPATAGSGPAATQDFSKLLSSVKEISDIVQR

In some embodiments, an ABL1 fusion polypeptide of the disclosure is a BCR-ABL1 fusion polypeptide comprising the amino acid sequence of SEQ ID NO: 34, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 34.

(SEQ ID NO: 34)
MVDPVGFAEAWKAQFPDSEPPRMELRSVGDIEQELERCKASIRRLEQEVNQERFRMIYLQTLLAKEKK
SYDRQRWGERRAAQAPDGASEPRASASRPQPAPADGADPPPAEEPEARPDGEGSPGKARPGTARRPGA
AASGERDDRGPPASVAALRSNFERIRKGHGQPGADAEKPFYVNVEFHHERGLVKVNDKEVSDRISSLG
SQAMQMERKKSQHGAGSSVGDASRPPYRGRSSESSCGVDGDYEDAELNPRFLKDNLIDANGGSRPPWP
PLEYQPYQSIYVGGMMEGEGKGPLLRSQSTSEQEKRLTWPRRSYSPRSFEDCGGGYTPDCSSNENLTS
SEEDFSSGQSSRVSPSPTTYRMFRDKSRSPSQNSQQSFDSSSPPTPQCHKRHRHCPVVVSEATIVGVR
KTGQIWPNDGEGAFHGDADGSFGTPPGYGCAADRAEEQRRHQDGLPYIDDSPSSSPHLSSKGRGSRDA
LVSGALESTKASELDLEKGLEMRKWVLSGILASEETYLSHLEALLLPMKPLKAAATTSQPVLTSQQIE
TIFFKVPELYEIHKEFYDGLFPRVQQWSHQQRVGDLFQKLASQLGVYRAFVDNYGVAMEMAEKCCQAN
AQFAEISENLRARSNKDAKDPTTKNSLETLLYKPVDRVTRSTLVLHDLLKHTPASHPDHPLLQDALRI
SQNFLSSINEEITPRRQSMTVKKGEHRQLLKDSEMVELVEGARKLRHVFLFTDLLLCTKLKKQSGGKT
QQYDCKWYIPLTDLSFQMVDELEAVPNIPLVPDEELDALKIKISQIKNDIQREKRANKGSKATERLKK
KLSEQESLLLLMSPSMAFRVHSRNGKSYTFLISSDYERAEWRENIREQQKKCFRSFSLTSVELQMLTN
SCVKLQTVHSIPLTINKEEALQRPVASDFEPQGLSEAARWNSKENLLAGPSENDPNLFVALYDEVASG
DNTLSITKGEKLRVLGYNHNGEWCEAQTKNGQGWVPSNYITPVNSLEKHSWYHGPVSRNAAEYLLSSG
INGSFLVRESESSPGQRSISLRYEGRVYHYRINTASDGKLYVSSESRFNTLAELVHHHSTVADGLITT
LHYPAPKRNKPTVYGVSPNYDKWEMERTDITMKHKLGGGQYGEVYEGVWKKYSLTVAVKTLKEDTMEV
EEFLKEAAVMKEIKHPNLVQLLGVCTREPPFYIITEFMTYGNLLDYLRECNRQEVNAVVLLYMATQIS
SAMEYLEKKNFIHRDLAARNCLVGENHLVKVADFGLSRLMTGDTYTAHAGAKFPIKWTAPESLAYNKE
SIKSDVWAFGVLLWEIATYGMSPYPGIDLSQVYELLEKDYRMERPEGCPEKVYELMRACWQWNPSDRP
SFAEIHQAFETMFQESSISDEVEKELGKQGVRGAVSTLLQAPELPTKTRTSRRAAEHRDTTDVPEMPH
SKGQGESDPLDHEPAVSPLLPRKERGPPEGGLNEDERLLPKDKKTNLESALIKKKKKTAPTPPKRSSS
FREMDGQPERRGAGEEEGRDISNGALAFTPLDTADPAKSPKPSNGAGVPNGALRESGGSGERSPHLWK
KSSTLTSSRLATGEEEGGGSSSKRFLRSCSASCVPHGAKDTEWRSVTLPRDLQSTGRQFDSSTFGGHK
SEKPALPRKRAGENRSDQVTRGTVTPPPRLVKKNEEAADEVEKDIMESSPGSSPPNLTPKPLRRQVTV
APASGLPHKEEAGKGSALGTPAAAEPVTPTSKAGSGAPGGTSKGPAEESRVRRHKHSSESPGRDKGKL
SRLKPAPPPPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPERIASGAIT
KGVVLDSTEALCLAISRNSEQMASHSAVLEAGKNLYTFCVSYVDSIQQMRNKFAFREAINKLENNLRE
LQICPATAGSGPAATQDFSKLLSSVKEISDIVQR

In some embodiments, an ABL1 fusion polypeptide of the disclosure is a BCR-ABL1 fusion polypeptide comprising the amino acid sequence of SEQ ID NO: 35, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 35.

(SEQ ID NO: 35)
MVDPVGFAEAWKAQFPDSEPPRMELRSVGDIEQELERCKASIRRLEQEVNQERFRMIYLQTLLAKEKK
SYDRQRWGERRAAQAPDGASEPRASASRPQPAPADGADPPPAEEPEARPDGEGSPGKARPGTARRPGA
AASGERDDRGPPASVAALRSNFERIRKGHGQPGADAEKPFYVNVEFHHERGLVKVNDKEVSDRISSLG
SQAMQMERKKSQHGAGSSVGDASRPPYRGRSSESSCGVDGDYEDAELNPRFLKDNLIDANGGSRPPWP
PLEYQPYQSIYVGGMMEGEGKGPLLRSQSTSEQEKRLTWPRRSYSPRSFEDCGGGYTPDCSSNENLTS
SEEDFSSGQSSRVSPSPTTYRMFRDKSRSPSQNSQQSFDSSSPPTPQCHKRHRHCPVVVSEATIVGVR
KTGQIWPNDGEGAFHGDADGSFGTPPGYGCAADRAEEQRRHQDGLPYIDDSPSSSPHLSSKGRGSRDA
LVSGALESTKASELDLEKGLEMRKWVLSGILASEETYLSHLEALLLPMKPLKAAATTSQPVLTSQQIE
TIFFKVPELYEIHKEFYDGLFPRVQQWSHQQRVGDLFQKLASQLGVYRAFVDNYGVAMEMAEKCCQAN
AQFAEISENLRARSNKDAKDPTTKNSLETLLYKPVDRVTRSTLVLHDLLKHTPASHPDHPLLQDALRI
SQNFLSSINEEITPRRQSMTVKKGEHRQLLKDSFMVELVEGARKLRHVFLFTDLLLCTKLKKQSGGKT
QQYDCKWYIPLTDLSFQMVDELEAVPNIPLVPDEELDALKIKISQIKNDIQREKRANKGSKATERLKK
KLSEQESLLLLMSPSMAFRVHSRNGKSYTFLISSDYERAEWRENIREQQKKCFRSFSLTSVELQMLTN
SCVKLQTVHSIPLTINKEDDESPGLYGFLNVIVHSATGFKQSSKALQRPVASDFEPQGLSEAARWNSK
ENLLAGPSENDPNLFVALYDFVASGDNTLSITKGEKLRVLGYNHNGEWCEAQTKNGQGWVPSNYITPV
NSLEKHSWYHGPVSRNAAEYLLSSGINGSFLVRESESSPGQRSISLRYEGRVYHYRINTASDGKLYVS
SESRFNTLAELVHHHSTVADGLITTLHYPAPKRNKPTVYGVSPNYDKWEMERTDITMKHKLGGGQYGE
VYEGVWKKYSLTVAVKTLKEDTMEVEEFLKEAAVMKEIKHPNLVQLLGVCTREPPFYIITEFMTYGNL
LDYLRECNRQEVNAVVLLYMATQISSAMEYLEKKNFIHRDLAARNCLVGENHLVKVADFGLSRLMTGD
TYTAHAGAKFPIKWTAPESLAYNKESIKSDVWAFGVLLWEIATYGMSPYPGIDLSQVYELLEKDYRME
RPEGCPEKVYELMRACWQWNPSDRPSFAEIHQAFETMFQESSISDEVEKELGKQGVRGAVSTLLQAPE
LPTKTRTSRRAAEHRDTTDVPEMPHSKGQGESDPLDHEPAVSPLLPRKERGPPEGGLNEDERLLPKDK
KTNLFSALIKKKKKTAPTPPKRSSSFREMDGQPERRGAGEEEGRDISNGALAFTPLDTADPAKSPKPS
NGAGVPNGALRESGGSGERSPHLWKKSSTLTSSRLATGEEEGGGSSSKRELRSCSASCVPHGAKDTEW
RSVTLPRDLQSTGRQFDSSTFGGHKSEKPALPRKRAGENRSDQVTRGTVTPPPRLVKKNEEAADEVFK
DIMESSPGSSPPNLTPKPLRRQVTVAPASGLPHKEEAGKGSALGTPAAAEPVTPTSKAGSGAPGGTSK
GPAEESRVRRHKHSSESPGRDKGKLSRLKPAPPPPPAASAGKAGGKPSQSPSQEAAGEAVLGAKTKAT
SLVDAVNSDAAKPSQPGEGLKKPVLPATPKPQSAKPSGTPISPAPVPSTLPSASSALAGDQPSSTAFI
PLISTRVSLRKTRQPPERIASGAITKGVVLDSTEALCLAISRNSEQMASHSAVLEAGKNLYTFCVSYV
DSIQQMRNKFAFREAINKLENNLRELQICPATAGSGPAATQDFSKLLSSVKEISDIVQR

In some embodiments, an ABL1 fusion polypeptide of the disclosure is a BCR-ABL1 fusion polypeptide comprising the amino acid sequence of SEQ ID NO: 36, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 36.

(SEQ ID NO: 36)
MVDPVGFAEAWKAQFPDSEPPRMELRSVGDIEQELERCKASIRRLEQEVNQERFRMIYLQTLLAKEKK
SYDRQRWGERRAAQAPDGASEPRASASRPQPAPADGADPPPAEEPEARPDGEGSPGKARPGTARRPGA
AASGERDDRGPPASVAALRSNFERIRKGHGQPGADAEKPFYVNVEFHHERGLVKVNDKEVSDRISSLG
SQAMQMERKKSQHGAGSSVGDASRPPYRGRSSESSCGVDGDYEDAELNPRFLKDNLIDANGGSRPPWP
PLEYQPYQSIYVGGMMEGEGKGPLLRSQSTSEQEKRLTWPRRSYSPRSFEDCGGGYTPDCSSNENLTS
SEEDFSSGQSSRVSPSPTTYRMFRDKSRSPSQNSQQSFDSSSPPTPQCHKRHRHCPVVVSEATIVGVR
KTGQIWPNDGEGAFHGDADGSFGTPPGYGCAADRAEEQRRHQDGLPYIDDSPSSSPHLSSKGRGSRDA
LVSGALESTKASELDLEKGLEMRKWVLSGILASEETYLSHLEALLLPMKPLKAAATTSQPVLTSQQIE
TIFFKVPELYEIHKEFYDGLFPRVQQWSHQQRVGDLFQKLASQLGVYRAFVDNYGVAMEMAEKCCQAN
AQFAEISENLRARSNKDAKDPTTKNSLETLLYKPVDRVTRSTLVLHDLLKHTPASHPDHPLLQDALRI
SQNFLSSINEEITPRRQSMTVKKGEHRQLLKDSEMVELVEGARKLRHVFLFTDLLLCTKLKKQSGGKT
QQYDCKWYIPLTDLSFQMVDELEAVPNIPLVPDEELDALKIKISQIKNDIQREKRANKGSKATERLKK
KLSEQESLLLLMSPSMAFRVHSRNGKSYTFLISSDYERAEWRENIREQQKKCFRSFSLTSVELQMLTN
SCVKLQTVHSIPLTINKEDDESPGLYGFLNVIVHSATGFKQSSKALQRPVASDFEPQGLSEAARWNSK
ENLLAGPSENDPNLFVALYDFVASGDNTLSITKGEKLRVLGYNHNGEWCEAQTKNGQGWVPSNYITPV
NSLEKHSWYHGPVSRNAAEYLLSSGINGSFLVRESESSPGQRSISLRYEGRVYHYRINTASDGKLYVS
SESRFNTLAELVHHHSTVADGLITTLHYPAPKRNKPTVYGVSPNYDKWEMERTDITMKHKLGGGQYGE
VYEGVWKKYSLTVAVKTLKEDTMEVEEFLKEAAVMKEIKHPNLVQLLGVCTREPPFYIITEFMTYGNL
LDYLRECNRQEVNAVVLLYMATQISSAMEYLEKKNFIHRDLAARNCLVGENHLVKVADFGLSRLMTGD
TYTAHAGAKFPIKWTAPESLAYNKESIKSDVWAFGVLLWEIATYGMSPYPGIDLSQVYELLEKDYRME
RPEGCPEKVYELMRACWQWNPSDRPSFAEIHQAFETMFQESSISDEVEKELGKQGVRGAVSTLLQAPE
LPTKTRTSRRAAEHRDTTDVPEMPHSKGQGESDPLDHEPAVSPLLPRKERGPPEGGLNEDERLLPKDK
KTNLFSALIKKKKKTAPTPPKRSSSFREMDGQPERRGAGEEEGRDISNGALAFTPLDTADPAKSPKPS
NGAGVPNGALRESGGSGERSPHLWKKSSTLTSSRLATGEEEGGGSSSKRELRSCSASCVPHGAKDTEW
RSVTLPRDLQSTGRQFDSSTFGGHKSEKPALPRKRAGENRSDQVTRGTVTPPPRLVKKNEEAADEVFK
DIMESSPGSSPPNLTPKPLRRQVTVAPASGLPHKEEAGKGSALGTPAAAEPVTPTSKAGSGAPGGTSK
GPAEESRVRRHKHSSESPGRDKGKLSRLKPAPPPPPAASAGKAGGKPSQSPSQEAAGEAVLGAKTKAT
SLVDAVNSDAAKPSQPGEGLKKPVLPATPKPQSAKPSGTPISPAPVPSTLPSASSALAGDQPSSTAFI
PLISTRVSLRKTRQPPERIASGAITKGVVLDSTEALCLAISRNSEQMASHSAVLEAGKNLYTFCVSYV
DSIQQMRNKFAFREAINKLENNLRELQICPATAGSGPAATQDFSKLLSSVKEISDIVQR

In some embodiments, an ABL1 fusion polypeptide of the disclosure is a BCR-ABL1 fusion polypeptide comprising the amino acid sequence of SEQ ID NO: 37, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 37.

(SEQ ID NO: 37)
MVDPVGFAEAWKAQFPDSEPPRMELRSVGDIEQELERCKASIRRLEQEVNQERFRMIYLQTLLAKEKK
SYDRQRWGERRAAQAPDGASEPRASASRPQPAPADGADPPPAEEPEARPDGEGSPGKARPGTARRPGA
AASGERDDRGPPASVAALRSNFERIRKGHGQPGADAEKPFYVNVEFHHERGLVKVNDKEVSDRISSLG
SQAMQMERKKSQHGAGSSVGDASRPPYRGRSSESSCGVDGDYEDAELNPRFLKDNLIDANGGSRPPWP
PLEYQPYQSIYVGGMMEGEGKGPLLRSQSTSEQEKRLTWPRRSYSPRSFEDCGGGYTPDCSSNENLTS
SEEDFSSGQSSRVSPSPTTYRMFRDKSRSPSQNSQQSFDSSSPPTPQCHKRHRHCPVVVSEATIVGVR
KTGQIWPNDGEGAFHGDADGSFGTPPGYGCAADRAEEQRRHQDGLPYIDDSPSSSPHLSSKGRGSRDA
LVSGALESTKASELDLEKGLEMRKWVLSGILASEETYLSHLEALLLPMKPLKAAATTSQPVLTSQQIE
TIFFKVPELYEIHKEFYDGLFPRVQQWSHQQRVGDLFQKLASQLGVYRAFVDNYGVAMEMAEKCCQAN
AQFAEISENLRARSNKDAKDPTTKNSLETLLYKPVDRVTRSTLVLHDLLKHTPASHPDHPLLQDALRI
SQNFLSSINEEITPRRQSMTVKKGEHRQLLKDSEMVELVEGARKLRHVFLFTDLLLCTKLKKQSGGKT
QQYDCKWYIPLTDLSFQMVDELEAVPNIPLVPDEELDALKIKISQIKNDIQREKRANKGSKATERLKK
KLSEQESLLLLMSPSMAFRVHSRNGKSYTFLISSDYERAEWRENIREQQKKCFRSFSLTSVELQMLTN
SCVKLQTVHSIPLTINKEDDESPGLYGFLNVIVHSATGFKQSSKALQRPVASDFEPQGLSEAARWNSK
ENLLAGPSENDPNLFVALYDFVASGDNTLSITKGEKLRVLGYNHNGEWCEAQTKNGQGWVPSNYITPV
NSLEKHSWYHGPVSRNAAEYLLSSGINGSFLVRESESSPGQRSISLRYEGRVYHYRINTASDGKLYVS
SESRFNTLAELVHHHSTVADGLITTLHYPAPKRNKPTVYGVSPNYDKWEMERTDITMKHKLGGGQYGE
VYEGVWKKYSLTVAVKTLKEDTMEVEEFLKEAAVMKEIKHPNLVQLLGVCTREPPFYIITEFMTYGNL
LDYLRECNRQEVNAVVLLYMATQISSAMEYLEKKNFIHRDLAARNCLVGENHLVKVADFGLSRLMTGD
TYTAHAGAKFPIKWTAPESLAYNKESIKSDVWAFGVLLWEIATYGMSPYPGIDLSQVYELLEKDYRME
RPEGCPEKVYELMRACWQWNPSDRPSFAEIHQAFETMFQESSISDEVEKELGKQGVRGAVSTLLQAPE
LPTKTRTSRRAAEHRDTTDVPEMPHSKGQGESDPLDHEPAVSPLLPRKERGPPEGGLNEDERLLPKDK
KTNLESALIKKKKKTAPTPPKRSSSFREMDGQPERRGAGEEEGRDISNGALAFTPLDTADPAKSPKPS
NGAGVPNGALRESGGSGERSPHLWKKSSTLTSSRLATGEEEGGGSSSKRELRSCSASCVPHGAKDTEW
RSVTLPRDLQSTGRQFDSSTFGGHKSEKPALPRKRAGENRSDQVTRGTVTPPPRLVKKNEEAADEVFK
DIMESSPGSSPPNLTPKPLRRQVTVAPASGLPHKEEAGKGSALGTPAAAEPVTPTSKAGSGAPGGTSK
GPAEESRVRRHKHSSESPGRDKGKLSRLKPAPPPPPAASAGKAGGKPSQSPSQEAAGEAVLGAKTKAT
SLVDAVNSDAAKPSQPGEGLKKPVLPATPKPQSAKPSGTPISPAPVPSTLPSASSALAGDQPSSTAFI
PLISTRVSLRKTRQPPERIASGAITKGVVLDSTEALCLAISRNSEQMASHSAVLEAGKNLYTFCVSYV
DSIQQMRNKFAFREAINKLENNLRELQICPATAGSGPAATQDFSKLLSSVKEISDIVQR

In some embodiments, an ABL1 fusion polypeptide of the disclosure is a BCR-ABL1 fusion polypeptide comprising the amino acid sequence of SEQ ID NO: 38, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 38.

(SEQ ID NO: 38)
MVDPVGFAEAWKAQFPDSEPPRMELRSVGDIEQELERCKASIRRLEQEVNQERFRMIYLQTLLAKEKK
SYDRQRWGFRRAAQAPDGASEPRASASRPQPAPADGADPPPAEEPEARPDGEGSPGKARPGTARRPGA
AASGERDDRGPPASVAALRSNFERIRKGHGQPGADAEKPFYVNVEFHHERGLVKVNDKEVSDRISSLG
SQAMQMERKKSQHGAGSSVGDASRPPYRGRSSESSCGVDGDYEDAELNPRFLKDNLIDANGGSRPPWP
PLEYQPYQSIYVGGMMEGEGKGPLLRSQSTSEQEKRLTWPRRSYSPRSFEDCGGGYTPDCSSNENLTS
SEEDFSSGQSSRVSPSPTTYRMFRDKSRSPSQNSQQSFDSSSPPTPQCHKRHRHCPVVVSEATIVGVR
KTGQIWPNDGEGAFHGDADGSFGTPPGYGCAADRAEEQRRHQDGLPYIDDSPSSSPHLSSKGRGSRDA
LVSGALESTKASELDLEKGLEMRKWVLSGILASEETYLSHLEALLLPMKPLKAAATTSQPVLTSQQIE
TIFFKVPELYEIHKEFYDGLFPRVQQWSHQQRVGDLFQKLASQLGVYRAFVDNYGVAMEMAEKCCQAN
AQFAEISENLRARSNKDAKDPTTKNSLETLLYKPVDRVTRSTLVLHDLLKHTPASHPDHPLLQDALRI
SQNFLSSINEEITPRRQSMTVKKGEHRQLLKDSEMVELVEGARKLRHVFLFTDLLLCTKLKKQSGGKT
QQYDCKWYIPLTDLSFQMVDELEAVPNIPLVPDEELDALKIKISQIKNDIQREKRANKGSKATERLKK
KLSEQESLLLLMSPSMAFRVHSRNGKSYTFLISSDYERAEWRENIREQQKKCFRSFSLTSVELQMLTN
SCVKLQTVHSIPLTINKEDDESPGLYGFLNVIVHSATGFKQSSKALQRPVASDFEPQGLSEAARWNSK
ENLLAGPSENDPNLFVALYDFVASGDNTLSITKGEKLRVLGYNHNGEWCEAQTKNGQGWVPSNYITPV
NSLEKHSWYHGPVSRNAAEYLLSSGINGSFLVRESESSPGQRSISLRYEGRVYHYRINTASDGKLYVS
SESRFNTLAELVHHHSTVADGLITTLHYPAPKRNKPTVYGVSPNYDKWEMERTDITMKHKLGGGQYGE
VYEGVWKKYSLTVAVKTLKEDTMEVEEFLKEAAVMKEIKHPNLVQLLGVCTREPPFYIITEFMTYGNL
LDYLRECNRQEVNAVVLLYMATQISSAMEYLEKKNFIHRDLAARNCLVGENHLVKVADEGLSRLMTGD
TYTAHAGAKFPIKWTAPESLAYNKESIKSDVWAFGVLLWEIATYGMSPYPGIDLSQVYELLEKDYRME
RPEGCPEKVYELMRACWQWNPSDRPSFAEIHQAFETMFQESSISDEVEKELGKQGVRGAVSTLLQAPE
LPTKTRTSRRAAEHRDTTDVPEMPHSKGQGESDPLDHEPAVSPLLPRKERGPPEGGLNEDERLLPKDK
KTNLFSALIKKKKKTAPTPPKRSSSFREMDGQPERRGAGEEEGRDISNGALAFTPLDTADPAKSPKPS
NGAGVPNGALRESGGSGFRSPHLWKKSSTLTSSRLATGEEEGGGSSSKRFLRSCSASCVPHGAKDTEW
RSVTLPRDLQSTGRQFDSSTFGGHKSEKPALPRKRAGENRSDQVTRGTVTPPPRLVKKNEEAADEVFK
DIMESSPGSSPPNLTPKPLRRQVTVAPASGLPHKEEAGKGSALGTPAAAEPVTPTSKAGSGAPGGTSK
GPAEESRVRRHKHSSESPGRDKGKLSRLKPAPPPPPAASAGKAGGKPSQSPSQEAAGEAVLGAKTKAT
SLVDAVNSDAAKPSQPGEGLKKPVLPATPKPQSAKPSGTPISPAPVPSTLPSASSALAGDQPSSTAFI
PLISTRVSLRKTRQPPERIASGAITKGVVLDSTEALCLAISRNSEQMASHSAVLEAGKNLYTFCVSYV
DSIQQMRNKFAFREAINKLENNLRELQICPATAGSGPAATQDFSKLLSSVKEISDIVQR

In some embodiments, an ABL1 fusion polypeptide of the disclosure is a BCR-ABL1 fusion polypeptide comprising the amino acid sequence of SEQ ID NO: 39, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 39.

(SEQ ID NO: 39)
MVDPVGFAEAWKAQFPDSEPPRMELRSVGDIEQELERCKASIRRLEQEVNQERFRMIYLQTLLAKEKK
SYDRQRWGERRAAQAPDGASEPRASASRPQPAPADGADPPPAEEPEARPDGEGSPGKARPGTARRPGA
AASGERDDRGPPASVAALRSNFERIRKGHGQPGADAEKPFYVNVEFHHERGLVKVNDKEVSDRISSLG
SQAMQMERKKSQHGAGSSVGDASRPPYRGRSSESSCGVDGDYEDAELNPRFLKDNLIDANGGSRPPWP
PLEYQPYQSIYVGGMMEGEGKGPLLRSQSTSEQEKRLTWPRRSYSPRSFEDCGGGYTPDCSSNENLTS
SEEDFSSGQSSRVSPSPTTYRMFRDKSRSPSQNSQQSFDSSSPPTPQCHKRHRHCPVVVSEATIVGVR
KTGQIWPNDGEGAFHGDADGSFGTPPGYGCAADRAEEQRRHQDGLPYIDDSPSSSPHLSSKGRGSRDA
LVSGALESTKASELDLEKGLEMRKWVLSGILASEETYLSHLEALLLPMKPLKAAATTSQPVLTSQQIE
TIFFKVPELYEIHKEFYDGLFPRVQQWSHQQRVGDLFQKLASQLGVYRAFVDNYGVAMEMAEKCCQAN
AQFAEISENLRARSNKDAKDPTTKNSLETLLYKPVDRVTRSTLVLHDLLKHTPASHPDHPLLQDALRI
SQNFLSSINEEITPRRQSMTVKKGEHRQLLKDSEMVELVEGARKLRHVFLFTDLLLCTKLKKQSGGKT
QQYDCKWYIPLTDLSFQMVDELEAVPNIPLVPDEELDALKIKISQIKNDIQREKRANKGSKATERLKK
KLSEQESLLLLMSPSMAFRVHSRNGKSYTFLISSDYERAEWRENIREQQKKCFRSFSLTSVELQMLTN
SCVKLQTVHSIPLTINKEDDESPGLYGFLNVIVHSATGFKQSSKALQRPVASDFEPQGLSEAARWNSK
ENLLAGPSENDPNLFVALYDFVASGDNTLSITKGEKLRVLGYNHNGEWCEAQTKNGQGWVPSNYITPV
NSLEKHSWYHGPVSRNAAEYLLSSGINGSFLVRESESSPGQRSISLRYEGRVYHYRINTASDGKLYVS
SESRFNTLAELVHHHSTVADGLITTLHYPAPKRNKPTVYGVSPNYDKWEMERTDITMKHKLGGGQYGE
VYEGVWKKYSLTVAVKTLKEDTMEVEEFLKEAAVMKEIKHPNLVQLLGVCTREPPFYIITEFMTYGNL
LDYLRECNRQEVNAVVLLYMATQISSAMEYLEKKNFIHRDLAARNCLVGENHLVKVADFGLSRLMTGD
TYTAHAGAKFPIKWTAPESLAYNKESIKSDVWAFGVLLWEIATYGMSPYPGIDLSQVYELLEKDYRME
RPEGCPEKVYELMRACWQWNPSDRPSFAEIHQAFETMFQESSISDEVEKELGKQGVRGAVSTLLQAPE
LPTKTRTSRRAAEHRDTTDVPEMPHSKGQGESDPLDHEPAVSPLLPRKERGPPEGGLNEDERLLPKDK
KTNLFSALIKKKKKTAPTPPKRSSSFREMDGQPERRGAGEEEGRDISNGALAFTPLDTADPAKSPKPS
NGAGVPNGALRESGGSGFRSPHLWKKSSTLTSSRLATGEEEGGGSSSKRFLRSCSASCVPHGAKDTEW
RSVTLPRDLQSTGRQFDSSTFGGHKSEKPALPRKRAGENRSDQVTRGTVTPPPRLVKKNEEAADEVFK
DIMESSPGSSPPNLTPKPLRRQVTVAPASGLPHKEEAGKGSALGTPAAAEPVTPTSKAGSGAPGGTSK
GPAEESRVRRHKHSSESPGRDKGKLSRLKPAPPPPPAASAGKAGGKPSQSPSQEAAGEAVLGAKTKAT
SLVDAVNSDAAKPSQPGEGLKKPVLPATPKPQSAKPSGTPISPAPVPSTLPSASSALAGDQPSSTAFI
PLISTRVSLRKTRQPPERIASGAITKGVVLDSTEALCLAISRNSEQMASHSAVLEAGKNLYTFCVSYV
DSIQQMRNKFAFREAINKLENNLRELQICPATAGSGPAATQDFSKLLSSVKEISDIQR

In some embodiments, an ABL1 fusion polypeptide of the disclosure is a NUP214-ABL1 fusion polypeptide comprising the amino acid sequence of SEQ ID NO: 40, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 40.

(SEQ ID NO: 40)
MGDEMDAMIPEREMKDFQFRALKKVRIFDSPEELPKERSSLLAVSNKYGLVFAGGASGLQIFPTKNLL
IQNKPGDDPNKIVDKVQGLLVPMKFPIHHLALSCDNLTLSACMMSSEYGSIIAFFDVRTFSNEAKQQK
RPFAYHKLLKDAGGMVIDMKWNPTVPSMVAVCLADGSIAVLQVTETVKVCATLPSTVAVTSVCWSPKG
KQLAVGKQNGTVVQYLPTLQEKKVIPCPPFYESDHPVRVLDVLWIGTYVFAIVYAAADGTLETSPDVV
MALLPKKEEKHPEIFVNFMEPCYGSCTERQHHYYLSYIEEWDLVLAASAASTEVSILARQSDQINWES
WLLEDSSRAELPVTDKSDDSLPMGVVVDYTNQVEITISDEKTLPPAPVLMLLSTDGVLCPFYMINQNP
GVKSLIKTPERLSLEGERQPKSPGSTPTTPTSSQAPQKLDASAAAAPASLPPSSPAAPIATESLLPAG
GAPTVFSFGSSSLKSSATVTGEPPSYSSGSDSSKAAPGPGPSTFSFVPPSKASLAPTPAASPVAPSAA
SFSFGSSGFKPTLESTPVPSVSAPNIAMKPSFPPSTSAVKVNLSEKFTAAATSTPVSSSQSAPPMSPF
SSASKPAASGPLSHPTPLSAPPSSVPLKSSVLPSPSGRSAQGSSSPVPSMVQKSPRITPPAAKPGSPQ
AKSLQPAVAEKQGHQWKDSDPVMAGIGEEIAHFQKELEELKARTSKACFQVGTSEEMKMLRTESDDLH
TFLLEIKETTESLHGDISSLKTTLLEGFAGVEEAREQNERNRDSGYLHLLYKRPLDPKSEAQLQEIRR
LHQYVKFAVQDVNDVLDLEWDQHLEQKKKQRHLLVPERETLENTLANNREIINQQRKRLNHLVDSLQQ
LRLYKQTSLWSLSSAVPSQSSIHSFDSDLESLCNALLKTTIESHTKSLPKVPAKLSPMKQAQLRNFLA
KRKTPPVRSTAPASLSRSAFLSQRYYEDLDEVSSTSSVSQSLESEDARTSCKDDEAVVQAPRHAPVVR
TPSIQPSLLPHAAPFAKSHLVHGSSPGVMGTSVATSASKIIPQGADSTMLATKTVKHGAPSPSHPISA
PQAAAAAALRRQMASQAPAVNTLTESTLKNVPQVVNVQELKNNPATPSTAMGSSVPYSTAKTPHPVLT
PVAANQAKQGSLINSLKPSGPTPASGQLSSGDKASGTAKIETAVTSTPSASGQFSKPFSFSPSGTGFN
FGIITPTPSSNFTAAQGATPSTKESSQPDAFSSGGGSKPSYEAIPESSPPSGITSASNTTPGEPAASS
SRPVAPSGTALSTTSSKLETPPSKLGELLFPSSLAGETLGSESGLRVGQADDSTKPTNKASSTSLTST
QPTKTSGVPSGENFTAPPVLGKHTEPPVTSSATTTSVAPPAATSTSSTAVFGSLPVTSAGSSGVISFG
GTSLSAGKTSFSFGSQQTNSTVPPSAPPPTTAATPLPTSFPTLSFGSLLSSATTPSLPMSAGRSTEEA
TSSALPEKPGDSEVSASAASLLEEQQSAQLPQAPPQTSDSVKKEPVLAQPAVSNSGTAASSTSLVALS
AEATPATTGVPDARTEAVPPASSESVPGQTAVTAAAISSAGPVAVETSSTPIASSTTSIVAPGPSAEA
AAFGTVTSGSSVFAQPPAASSSSAFNQLTNNTATAPSATPVFGQVAASTAPSLFGQQTGSTASTAAAT
PQVSSSGFSSPAFGTTAPGVFGQTTFGQASVFGQSASSAASVESFSQPGESSVPAFGQPASSTPTSTS
GSVFGAASSTSSSSSFSFGQSSPNTGGGLFGQSNAPAFGQSPGFGQGGSVFGGTSAATTTAATSGESF
CQASGFGSSNTGSVFGQAASTGGIVFGQQSSSSSGSVFGSGNTGRGGGFFSGLGGKPSQDAANKNPES
SASGGFGSTATSNTSNLFGNSGAKTFGGFASSSFGEQKPTGTFSSGGGSVASQGFGFSSPNKTGGFGA
APVFGSPPTFGGSPGFGGVPAFGSAPAFTSPLGSTGGKVEGEGTAAASAGGFGFGSSSNTTSEGTLAS
QNAPTFGSLSQQTSGFGTQSSGFSGFGSGTGGEKLRVLGYNHNGEWCEAQTKNGQGWVPSNYITPVNS
LEKHSWYHGPVSRNAAEYLLSSGINGSELVRESESSPGQRSISLRYEGRVYHYRINTASDGKLYVSSE
SRFNTLAELVHHHSTVADGLITTLHYPAPKRNKPTVYGVSPNYDKWEMERTDITMKHKLGGGQYGEVY
EGVWKKYSLTVAVKTLKEDTMEVEEFLKEAAVMKEIKHPNLVQLLGVCTREPPFYIITEFMTYGNLLD
YLRECNRQEVNAVVLLYMATQISSAMEYLEKKNFIHRDLAARNCLVGENHLVKVADEGLSRLMTGDTY
TAHAGAKFPIKWTAPESLAYNKESIKSDVWAFGVLLWEIATYGMSPYPGIDLSQVYELLEKDYRMERP
EGCPEKVYELMRACWQWNPSDRPSFAEIHQAFETMFQESSISDEVEKELGKQGVRGAVSTLLQAPELP
TKTRTSRRAAEHRDTTDVPEMPHSKGQGESDPLDHEPAVSPLLPRKERGPPEGGLNEDERLLPKDKKT
NLFSALIKKKKKTAPTPPKRSSSFREMDGQPERRGAGEEEGRDISNGALAFTPLDTADPAKSPKPSNG
AGVPNGALRESGGSGFRSPHLWKKSSTLTSSRLATGEEEGGGSSSKRELRSCSASCVPHGAKDTEWRS
VTLPRDLQSTGRQFDSSTFGGHKSEKPALPRKRAGENRSDQVTRGTVTPPPRLVKKNEEAADEVFKDI
MESSPGSSPPNLTPKPLRRQVTVAPASGLPHKEEAGKGSALGTPAAAEPVTPTSKAGSGAPGGTSKGP
AEESRVRRHKHSSESPGRDKGKLSRLKPAPPPPPAASAGKAGGKPSQSPSQEAAGEAVLGAKTKATSL
VDAVNSDAAKPSQPGEGLKKPVLPATPKPQSAKPSGTPISPAPVPSTLPSASSALAGDQPSSTAFIPL
ISTRVSLRKTRQPPERIASGAITKGVVLDSTEALCLAISRNSEQMASHSAVLEAGKNLYTFCVSYVDS
IQQMRNKFAFREAINKLENNLRELQICPATAGSGPAATQDFSKLLSSVKEISDIVQR

In some embodiments, an ABL1 fusion polypeptide of the disclosure is an ABL1-AIF1L fusion polypeptide comprising the amino acid sequence of SEQ ID NO: 41, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 41.

(SEQ ID NO: 41)
MLEICLKLVGCKSKKGLSSSSSCYLEEALQRPVASDFEPQGLSEAARWNSKENLLAGPSENDPNLFVA
LYDFVASGDNTLSITKGEKLRVLGYNHNGEWCEAQTKNGQGWVPSNYITPVNSLEKHSWYHGPVSRNA
AEYLLSSGINGSFLVRESESSPGQRSISLRYEGRVYHYRINTASDGKLYVSSESRFNTLAELVHHHST
VADGLITTLHYPAPKRNKPTVYGVSPNYDKWEMERTDITMKHKLGGGQYGEVYEGVWKKYSLTVAVKT
LKEDTMEVEEFLKEAAVMKEIKHPNLVQLLGVCTREPPFYIITEFMTYGNLLDYLRECNRQEVNAVVL
LYMATQISSAMEYLEKKNFIHRDLAARNCLVGENHLVKVADFGLSRLMTGDTYTAHAGAKFPIKWTAP
ESLAYNKESIKSDVWAFGVLLWEIATYGMSPYPGIDLSQVYELLEKDYRMERPEGCPEKVYELMRACW
QWNPSDRPSFAEIHQAFETMFQESSISDEVEKELGKQGVRGAVSTLLQAPELPTKTRTSRRAAEHRDT
TDVPEMPHSKGQGESEKYMEFDLNNEGEIDLMSLKRMMEKLGVPKTHLEMKKMISEVTGGVSDTISYR
DFVNMMLGKRSAVLKLVMMFEGKANESSPKPVGPPPERDIASLP

In some embodiments, an ABL1 fusion polypeptide of the disclosure is a RCSD1-ABL1 fusion polypeptide comprising the amino acid sequence of SEQ ID NO: 42, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 42.

(SEQ ID NO: 42)
MEERPAETNANVDNSASPSVAQLAGRFREQAAAAKETPASKPTRRKPPCSLPLFPPKVDLGQNGEEKS
PPNASHPPKFKVKSSPLIEKLQLYVSSESRFNTLAELVHHHSTVADGLITTLHYPAPKRNKPTVYGVS
PNYDKWEMERTDITMKHKLGGGQYGEVYEGVWKKYSLTVAVKTLKEDTMEVEEFLKEAAVMKEIKHPN
LVQLLGVCTREPPFYIITEFMTYGNLLDYLRECNRQEVNAVVLLYMATQISSAMEYLEKKNFIHRDLA
ARNCLVGENHLVKVADFGLSRLMTGDTYTAHAGAKFPIKWTAPESLAYNKESIKSDVWAFGVLLWEIA
TYGMSPYPGIDLSQVYELLEKDYRMERPEGCPEKVYELMRACWQWNPSDRPSFAEIHQAFETMFQESS
ISDEVEKELGKQGVRGAVSTLLQAPELPTKTRTSRRAAEHRDTTDVPEMPHSKGQGESDPLDHEPAVS
PLLPRKERGPPEGGLNEDERLLPKDKKTNLFSALIKKKKKTAPTPPKRSSSFREMDGQPERRGAGEEE
GRDISNGALAFTPLDTADPAKSPKPSNGAGVPNGALRESGGSGFRSPHLWKKSSTLTSSRLATGEEEG
GGSSSKRFLRSCSASCVPHGAKDTEWRSVTLPRDLQSTGRQFDSSTFGGHKSEKPALPRKRAGENRSD
QVTRGTVTPPPRLVKKNEEAADEVFKDIMESSPGSSPPNLTPKPLRRQVTVAPASGLPHKEEAGKGSA
LGTPAAAEPVTPTSKAGSGAPGGTSKGPAEESRVRRHKHSSESPGRDKGKLSRLKPAPPPPPAASAGK
AGGKPSQSPSQEAAGEAVLGAKTKATSLVDAVNSDAAKPSQPGEGLKKPVLPATPKPQSAKPSGTPIS
PAPVPSTLPSASSALAGDQPSSTAFIPLISTRVSLRKTRQPPERIASGAITKGVVLDSTEALCLAISR
NSEQMASHSAVLEAGKNLYTFCVSYVDSIQQMRNKFAFREAINKLENNLRELQICPATAGSGPAATQD
FSKLLSSVKEISDIVQR

In some embodiments, an ABL1 fusion polypeptide of the disclosure is encoded by an ABL1 fusion nucleic acid molecule as listed in Table 2 in Example 1 herein, e.g., comprising the corresponding nucleotide sequence as listed in Table 2 in Example 1 herein (or a sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology thereto). In some embodiments, an ABL1 fusion polypeptide of the disclosure comprises an ABL1 fusion polypeptide amino acid sequence as listed in Table 2 in Example 1 herein (or a sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology thereto).

### Exemplary ABL1 Fusion Polypeptides

In some embodiments, an ABL1 fusion polypeptide of the disclosure is an EHMT1-ABL1 fusion polypeptide, wherein the EHMT1-ABL1 fusion polypeptide: is encoded by an ABL1 fusion nucleic acid molecule comprising, in 5′ to 3′ direction, at least a portion of an EHMT1 gene fused to at least a portion of an ABL1 gene; is encoded by an ABL1 fusion nucleic acid molecule comprising or resulting from a breakpoint within intron 4 of an EHMT1 gene and/or a breakpoint within intron 1 of an ABL1 gene; comprises a fusion between an amino acid sequence encoded by exon 4, or a portion thereof, of an EHMT1 gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr9: 140630475-140630606 and/or a 3′ breakpoint within chromosomal coordinates chr9: 133729408-133729648; comprises, in the N- to C-terminus direction, an amino acid sequence encoded by exons 1-3 and exon 4, or a portion thereof, of an EHMT1 gene, fused to an amino acid sequence encoded by exon 2, or a portion thereof, and exons 3-11 of an ABL1 gene; is encoded by an ABL1 fusion nucleic acid molecule comprising, in the 5′ to 3′ direction, exons 1-3 and exon 4, or a portion thereof, of an EHMT1 gene, fused to exon 2, or a portion thereof, and exons 3-11 of an ABL1 gene; comprises the amino acid sequence of SEQ ID NO: 23, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 23; and/or is encoded by the nucleotide sequence of SEQ ID NO: 2, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 2. In some embodiments, the EHMT1-ABL1 fusion polypeptide comprises the ABL1 kinase domain, or a portion of the ABL1 kinase domain having kinase activity.

In some embodiments, an ABL1 fusion polypeptide of the disclosure is a MED27-ABL1 fusion polypeptide, wherein the MED27-ABL1 fusion polypeptide: is encoded by an ABL1 fusion nucleic acid molecule comprising, in 5′ to 3′ direction, at least a portion of a MED27 gene fused to at least a portion of an ABL1 gene; is encoded by an ABL1 fusion nucleic acid molecule comprising or resulting from a breakpoint within intron 4 of a MED27 gene and/or a breakpoint within intron 4 of an ABL1 gene; comprises a fusion between an amino acid sequence encoded by exon 4, or a portion thereof, of a MED27 gene and an amino acid sequence encoded by exon 5, or a portion thereof, of an ABL1 gene; is encoded by an ABL1 fusion nucleic acid molecule comprising or resulting from a 5′ breakpoint within chromosomal coordinates chr9: 134774423-134776006 and/or a 3′ breakpoint within chromosomal coordinates chr9: 133747364-133747762; comprises, in the N- to C-terminus direction, an amino acid sequence encoded by exons 1-3 and exon 4, or a portion thereof, of a MED27 gene, fused to an amino acid sequence encoded by exon 5, or a portion thereof, and exons 6-11 of an ABL1 gene; is encoded by an ABL1 fusion nucleic acid molecule comprising, in the 5′ to 3′ direction, exons 1-3 and exon 4, or a portion thereof, of a MED27 gene, fused to exon 5, or a portion thereof, and exons 6-11 of an ABL1 gene; comprises the amino acid sequence of SEQ ID NO: 24, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 24; and/or is encoded by the nucleotide sequence of SEQ ID NO: 3, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 3. In some embodiments, the MED27-ABL1 fusion polypeptide comprises the ABL1 kinase domain, or a portion of the ABL1 kinase domain having kinase activity.

In some embodiments, an ABL1 fusion polypeptide of the disclosure is an RFFL-ABL1 fusion polypeptide, wherein the RFFL-ABL1 fusion polypeptide: is encoded by an ABL1 fusion nucleic acid molecule comprising, in 5′ to 3′ direction, at least a portion of an RFFL gene fused to at least a portion of an ABL1 gene; comprises a fusion between an amino acid sequence encoded by exon 5, or a portion thereof, of an RFFL gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr17: 33343379-33343419 and/or a 3′ breakpoint within chromosomal coordinates chr9: 133729485-133729525; comprises, in the N- to C-terminus direction, an amino acid sequence encoded by exons 1-4 and exon 5, or a portion thereof, of an RFFL gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-4 and exon 5, or a portion thereof, of an RFFL gene, fused to exon 2, or a portion thereof, and exons 3-11 of an ABL1 gene; comprises the amino acid sequence of SEQ ID NO: 25, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 25; and/or is encoded by the nucleotide sequence of SEQ ID NO: 4, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 4. In some embodiments, the RFFL-ABL1 fusion polypeptide comprises the ABL1 kinase domain, or a portion of the ABL1 kinase domain having kinase activity.

In some embodiments, an ABL1 fusion polypeptide of the disclosure is an SLC27A4-ABL1 fusion polypeptide, wherein the SLC27A4-ABL1 fusion polypeptide: is encoded by an ABL1 fusion nucleic acid molecule comprising, in 5′ to 3′ direction, at least a portion of an SLC27A4 gene fused to at least a portion of an ABL1 gene; is encoded by an ABL1 fusion nucleic acid molecule comprising or resulting from a breakpoint within intron 12 of an SLC27A4 gene and/or a breakpoint within intron 5 of an ABL1 gene; comprises a fusion between an amino acid sequence encoded by exon 12, or a portion thereof, of an SLC27A4 gene and an amino acid sequence encoded by exon 6, or a portion thereof, of an ABL1 gene; is encoded by an ABL1 fusion nucleic acid molecule comprising or resulting from a 5′ breakpoint within chromosomal coordinates chr9: 131119603-131119801 and/or a 3′ breakpoint within chromosomal coordinates chr9: 133748211-133748501; comprises, in the N- to C-terminus direction, an amino acid sequence encoded by exons 1-11 and exon 12, or a portion thereof, of an SLC27A4 gene, fused to an amino acid sequence encoded by exon 6, or a portion thereof, and exons 7-11 of an ABL1 gene; is encoded by an ABL1 fusion nucleic acid molecule comprising, in 5′ to 3′ direction, exons 1-11 and exon 12, or a portion thereof, of an SLC27A4 gene, fused to exon 6, or a portion thereof, and exons 7-11 of an ABL1 gene; comprises the amino acid sequence of SEQ ID NO: 26, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 26; and/or is encoded by the nucleotide sequence of SEQ ID NO: 5, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 5. In some embodiments, the SLC27A4-ABL1 fusion polypeptide comprises the ABL1 kinase domain, or a portion of the ABL1 kinase domain having kinase activity.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 13 of a BCR gene and/or a breakpoint within intron 1 of an ABL1 gene; comprises a fusion between an amino acid sequence encoded by exon 13, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23632033-23632321 and/or a 3′ breakpoint within chromosomal coordinates chr9: 133591666-133591902; comprises, in the N- to C-terminus direction, an amino acid sequence encoded by exons 1-12 and exon 13, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-12 and exon 13, or a portion thereof, of a BCR gene, fused to exon 2, or a portion thereof, and exons 3-11 of an ABL1 gene; comprises the amino acid sequence of SEQ ID NO: 27, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 27; and/or is encoded by the nucleotide sequence of SEQ ID NO: 6, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 6. In some embodiments, the BCR-ABL1 fusion polypeptide comprises the ABL1 kinase domain, or a portion of the ABL1 kinase domain having kinase activity.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23634537-23634672 and/or a 3′ breakpoint within chromosomal coordinates chr9: 133672815-133673059; comprises, in then N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; comprises the amino acid sequence of SEQ ID NO: 28, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 28; and/or is encoded by the nucleotide sequence of SEQ ID NO: 7, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 7. In some embodiments, the BCR-ABL1 fusion polypeptide comprises the ABL1 kinase domain, or a portion of the ABL1 kinase domain having kinase activity.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23634041-23634323 and/or a 3′ breakpoint within chromosomal coordinates chr9: 133708182-133708567; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; comprises the amino acid sequence of SEQ ID NO: 29, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 29; and/or is encoded by the nucleotide sequence of SEQ ID NO: 8, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 8. In some embodiments, the BCR-ABL1 fusion polypeptide comprises the ABL1 kinase domain, or a portion of the ABL1 kinase domain having kinase activity.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23632683-23632948 and/or a 3′ breakpoint within chromosomal coordinates chr9: 133613427-133613626; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; comprises the amino acid sequence of SEQ ID NO: 30, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 30; and/or is encoded by the nucleotide sequence of SEQ ID NO: 9, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 9. In some embodiments, the BCR-ABL1 fusion polypeptide comprises the ABL1 kinase domain, or a portion of the ABL1 kinase domain having kinase activity.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23634011-23634460 and/or a 3′ breakpoint within chromosomal coordinates chr9: 133645693-133646017; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; comprises the amino acid sequence of SEQ ID NO: 32, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 32; and/or is encoded by the nucleotide sequence of SEQ ID NO: 11, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 11. In some embodiments, the BCR-ABL1 fusion polypeptide comprises the ABL1 kinase domain, or a portion of the ABL1 kinase domain having kinase activity.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23634211-23634632 and/or a 3′ breakpoint within chromosomal coordinates chr9: 133619101-133619395; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; comprises the amino acid sequence of SEQ ID NO: 33, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 33; and/or is encoded by the nucleotide sequence of SEQ ID NO: 12, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 12. In some embodiments, the BCR-ABL1 fusion polypeptide comprises the ABL1 kinase domain, or a portion of the ABL1 kinase domain having kinase activity.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 13 of a BCR gene and/or a breakpoint within intron 1 of an ABL1 gene; comprises a fusion between an amino acid sequence encoded by exon 13, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23631722-23631762 and/or a 3′ breakpoint within chromosomal coordinates chr9: 133729484-133729524; comprises, in the N- to C-terminus direction, an amino acid sequence encoded by exons 1-12 and exon 13, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-12 and exon 13, or a portion thereof, of a BCR gene, fused to exon 2, or a portion thereof, and exons 3-11 of an ABL1 gene; comprises the amino acid sequence of SEQ ID NO: 34, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 34; and/or is encoded by the nucleotide sequence of SEQ ID NO: 13, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 13. In some embodiments, the BCR-ABL1 fusion polypeptide comprises the ABL1 kinase domain, or a portion of the ABL1 kinase domain having kinase activity.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23633346-23634163 and/or a 3′ breakpoint within chromosomal coordinates chr9: 133609201-133609909; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; comprises the amino acid sequence of SEQ ID NO: 35, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 35; and/or is encoded by the nucleotide sequence of SEQ ID NO: 14, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 14. In some embodiments, the BCR-ABL1 fusion polypeptide comprises the ABL1 kinase domain, or a portion of the ABL1 kinase domain having kinase activity.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23633432-23633796 and/or a 3′ breakpoint within chromosomal coordinates chr9: 133623903-133624461; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; comprises the amino acid sequence of SEQ ID NO: 36, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 36; and/or is encoded by the nucleotide sequence of SEQ ID NO: 15, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 15. In some embodiments, the BCR-ABL1 fusion polypeptide comprises the ABL1 kinase domain, or a portion of the ABL1 kinase domain having kinase activity.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23633837-23634208 and/or a 3′ breakpoint within chromosomal coordinates chr9: 133665426-133665727; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; comprises the amino acid sequence of SEQ ID NO: 37, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 37; and/or is encoded by the nucleotide sequence of SEQ ID NO: 16, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 16. In some embodiments, the BCR-ABL1 fusion polypeptide comprises the ABL1 kinase domain, or a portion of the ABL1 kinase domain having kinase activity.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23632858-23633068 and/or a 3′ breakpoint within chromosomal coordinates chr9: 133701595-133701846; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; comprises the amino acid sequence of SEQ ID NO: 38, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 38; and/or is encoded by the nucleotide sequence of SEQ ID NO: 17, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 17. In some embodiments, the BCR-ABL1 fusion polypeptide comprises the ABL1 kinase domain, or a portion of the ABL1 kinase domain having kinase activity.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23633208-23633695 and/or a 3′ breakpoint within chromosomal coordinates chr9: 133702617-133702992; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; comprises the amino acid sequence of SEQ ID NO: 39, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 39; and/or is encoded by the nucleotide sequence of SEQ ID NO: 18, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 18. In some embodiments, the BCR-ABL1 fusion polypeptide comprises the ABL1 kinase domain, or a portion of the ABL1 kinase domain having kinase activity.

In some embodiments, an ABL1 fusion polypeptide of the disclosure is a NUP214-ABL1 fusion polypeptide, wherein the NUP214-ABL1 fusion polypeptide: is encoded by an ABL1 fusion nucleic acid molecule comprising, in 5′ to 3′ direction, at least a portion of a NUP214 gene fused to at least a portion of an ABL1 gene; comprises a fusion between an amino acid sequence encoded by exon 34, or a portion thereof, of a NUP214 gene and an amino acid sequence encoded by exon 3, or a portion thereof, of an ABL1 gene; is encoded by an ABL1 fusion nucleic acid molecule comprising or resulting from a 5′ breakpoint within chromosomal coordinates chr9: 134106039-134106079 and/or a 3′ breakpoint within chromosomal coordinates chr9: 133730222-133730262; comprises, in the N- to C-terminus direction, an amino acid sequence encoded by exons 1-33 and exon 34, or a portion thereof, of a NUP214 gene, fused to an amino acid sequence encoded by exon 3, or a portion thereof, and exons 4-11 of an ABL1 gene; is encoded by an ABL1 fusion nucleic acid molecule comprising, in 5′ to 3′ direction, exons 1-33 and exon 34, or a portion thereof, of a NUP214 gene, fused to exon 3, or a portion thereof, and exons 4-11 of an ABL1 gene; comprises the amino acid sequence of SEQ ID NO: 40, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 40; and/or is encoded by the nucleotide sequence of SEQ ID NO: 19, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 19. In some embodiments, the NUP214-ABL1 fusion polypeptide comprises the ABL1 kinase domain, or a portion of the ABL1 kinase domain having kinase activity.

In some embodiments, an ABL1 fusion polypeptide of the disclosure is an ABL1-AIF1L fusion polypeptide, wherein the ABL1-AIF1L fusion polypeptide: is encoded by an ABL1 fusion nucleic acid molecule comprising, in 5′ to 3′ direction, at least a portion of an ABL1 gene fused to at least a portion of an AIF1L gene; comprises a fusion between an amino acid sequence encoded by exon 10, or a portion thereof, of an ABL1 gene and an amino acid sequence encoded by exon 4, or a portion thereof, of an AIF1L gene; is encoded by an ABL1 fusion nucleic acid molecule comprising or resulting from a 5′ breakpoint within chromosomal coordinates chr9: 133755952-133755992 and/or a 3′ breakpoint within chromosomal coordinates chr9: 133989965-133990005; comprises, in the N- to C-terminus direction, an amino acid sequence encoded by exons 1-9 and exon 10, or a portion thereof, of an ABL1 gene, fused to an amino acid sequence encoded by exon 4, or a portion thereof, and exons 5-6 of an AIF1L gene; is encoded by an ABL1 fusion nucleic acid molecule comprising, in 5′ to 3′ direction, exons 1-9 and exon 10, or a portion thereof, of an ABL1 gene, fused to exon 4, or a portion thereof, and exons 5-6 of an AIF1L gene; comprises the amino acid sequence of SEQ ID NO: 41, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 41; and/or is encoded by the nucleotide sequence of SEQ ID NO: 20, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 20. In some embodiments, the ABL1-AIF1L fusion polypeptide comprises the ABL1 kinase domain, or a portion of the ABL1 kinase domain having kinase activity.

In some embodiments, an ABL1 fusion polypeptide of the disclosure is an RCSD1-ABL1 fusion polypeptide, wherein the RCSD1-ABL1 fusion polypeptide: is encoded by an ABL1 fusion nucleic acid molecule comprising, in 5′ to 3′ direction, at least a portion of an RCSD1 gene fused to at least a portion of an ABL1 gene; comprises a fusion between an amino acid sequence encoded by exon 4, or a portion thereof, of an RCSD1 gene and an amino acid sequence encoded by exon 4, or a portion thereof, of an ABL1 gene; is encoded by an ABL1 fusion nucleic acid molecule comprising or resulting from a 5′ breakpoint within chromosomal coordinates chr1: 167654668-167654708 and/or a 3′ breakpoint within chromosomal coordinates chr9: 133738173-133738213; comprises, in the N- to C-terminus direction, an amino acid sequence encoded by exons 1-3 and exon 4, or a portion thereof, of an RCSD1 gene, fused to an amino acid sequence encoded by exon 4, or a portion thereof, and exons 5-11 of an ABL1 gene; is encoded by an ABL1 fusion nucleic acid molecule comprising, in the 5′ to 3′ direction, exons 1-3 and exon 4, or a portion thereof, of an RCSD1 gene, fused to exon 4, or a portion thereof, and exons 5-11 of an ABL1 gene; comprises the amino acid sequence of SEQ ID NO: 42, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 42; and/or is encoded by the nucleotide sequence of SEQ ID NO: 21, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 21. In some embodiments, the RCSD1-ABL1 fusion polypeptide comprises the ABL1 kinase domain, or a portion of the ABL1 kinase domain having kinase activity.

In some embodiments, an ABL1 fusion polypeptide of the disclosure is an APOOL-ABL1 fusion polypeptide, wherein the APOOL-ABL1 fusion polypeptide: is encoded by an ABL1 fusion nucleic acid molecule comprising, in 5′ to 3′ direction, at least a portion of an APOOL gene fused to at least a portion of an ABL1 gene; is encoded by an ABL1 fusion nucleic acid molecule comprising or resulting from a breakpoint within intron 2 of an APOOL gene and/or a breakpoint within exon 2 of an ABL1 gene; comprises a fusion between an amino acid sequence encoded by exon 2, or a portion thereof, of an APOOL gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chrX: 84302705-84302904 and/or a 3′ breakpoint within chromosomal coordinates chr9: 133729584-133729766; comprises, in the N- to C-terminus direction, an amino acid sequence encoded by exon 1 and exon 2, or a portion thereof, of an APOOL gene, fused to an amino acid sequence encoded by exon 2, or a portion thereof, and exons 3-11 of an ABL1 gene; and/or is encoded by an ABL1 fusion nucleic acid molecule comprising, in the 5′ to 3′ direction, exon 1 and exon 2, or a portion thereof, of an APOOL gene, fused to exon 2, or a portion thereof, and exons 3-11 of an ABL1 gene. In some embodiments, the APOOL-ABL1 fusion polypeptide comprises the ABL1 kinase domain, or a portion of the ABL1 kinase domain having kinase activity.

In some embodiments, an ABL1 fusion polypeptide of the disclosure is an ABL1-DNAH2 fusion polypeptide, wherein the ABL1-DNAH2 fusion polypeptide: is encoded by an ABL1 fusion nucleic acid molecule comprising, in 5′ to 3′ direction, at least a portion of an ABL1 gene fused to at least a portion of a DNAH2 gene; comprises a fusion between an amino acid sequence encoded by exon 10, or a portion thereof, of an ABL1 gene and an amino acid sequence encoded by exon 4, or a portion thereof, of a DNAH2 gene; is encoded by an ABL1 fusion nucleic acid molecule comprising or resulting from a 5′ breakpoint within chromosomal coordinates chr9: 133755953-133755993 and/or a 3′ breakpoint within chromosomal coordinates chr17: 7636399-7636439; comprises, in the N- to C-terminus direction, an amino acid sequence encoded by exons 1-9 and exon 10, or a portion thereof, of an ABL1 gene, fused to an amino acid sequence encoded by exon 4, or a portion thereof, and exons 5-85 of a DNAH2 gene; and/or is encoded by an ABL1 fusion nucleic acid molecule comprising, in the 5′ to 3′ direction, exons 1-9 and exon 10, or a portion thereof, of an ABL1 gene, fused to exon 4, or a portion thereof, and exons 5-85 of a DNAH2 gene. In some embodiments, the ABL1-DNAH2 fusion polypeptide comprises the ABL1 kinase domain, or a portion of the ABL1 kinase domain having kinase activity.

In some embodiments, an ABL1 fusion polypeptide of the disclosure is a RHEX/C1orf186-ABL1 fusion polypeptide, wherein the RHEX/C1orf186-ABL1 fusion polypeptide: is encoded by an ABL1 fusion nucleic acid molecule comprising, in 5′ to 3′ direction, at least a portion of a RHEX/C1orf186-gene fused to at least a portion of an ABL1 gene; comprises a fusion between an amino acid sequence encoded by exon 1, or a portion thereof, of a RHEX/Clorf186 gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr1: 206288099-206288139 and/or a 3′ breakpoint within chromosomal coordinates chr9: 133729488-133729528; comprises, in the N- to C-terminus direction, an amino acid sequence encoded by exon 1, or a portion thereof, of a RHEX/Clorf186 gene, fused to an amino acid sequence encoded by exon 2, or a portion thereof, and exons 3-11 of an ABL1 gene; and/or is encoded by an ABL1 fusion nucleic acid molecule comprising, in 5′ to 3′ direction, exon 1, or a portion thereof, of a RHEX/Clorf186 gene, fused to exon 2, or a portion thereof, and exons 3-11 of an ABL1 gene. In some embodiments, the RHEX/C1orf186-ABL1 fusion polypeptide comprises the ABL1 kinase domain, or a portion of the ABL1 kinase domain having kinase activity.

In some embodiments, an ABL1 fusion polypeptide of the disclosure is a RALGPS1-ABL1 fusion polypeptide, wherein the RALGPS1-ABL1 fusion polypeptide: is encoded by an ABL1 fusion nucleic acid molecule comprising, in 5′ to 3′ direction, at least a portion of a RALGPS1 gene fused to at least a portion of an ABL1 gene; is encoded by an ABL1 fusion nucleic acid molecule comprising or resulting from a breakpoint within intron 1 of a RALGPS1 gene and/or a breakpoint within intron 2 of an ABL1 gene; comprises a fusion between an amino acid sequence encoded by exon 1, or a portion thereof, of a RALGPS1 gene and an amino acid sequence encoded by exon 3, or a portion thereof, of an ABL1 gene; is encoded by an ABL1 fusion nucleic acid molecule comprising or resulting from a 5′ breakpoint within chromosomal coordinates chr9: 129694023-129694282 and/or a 3′ breakpoint within chromosomal coordinates chr9: 133730118-133730529; comprises, in the N- to C-terminus direction, an amino acid sequence encoded by exon 1, or a portion thereof, of a RALGPS1 gene, fused to an amino acid sequence encoded by exon 3, or a portion thereof, and exons 4-11 of an ABL1 gene; and/or is encoded by an ABL1 fusion nucleic acid molecule comprising, in 5′ to 3′ direction, exon 1, or a portion thereof, of a RALGPS1 gene, fused to exon 3, or a portion thereof, and exons 4-11 of an ABL1 gene. In some embodiments, the RALGPS1-ABL1 fusion polypeptide comprises the ABL1 kinase domain, or a portion of the ABL1 kinase domain having kinase activity.

In some embodiments, an ABL1 fusion polypeptide of the disclosure is a TSC1-ABL1 fusion polypeptide, wherein the TSC1-ABL1 fusion polypeptide: is encoded by an ABL1 fusion nucleic acid molecule comprising, in 5′ to 3′ direction, at least a portion of a TSC1 gene fused to at least a portion of an ABL1 gene; comprises a fusion between an amino acid sequence encoded by exon 19, or a portion thereof, of a TSC1 gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr9:135,776,998-135,777,038 and/or a 3′ breakpoint within chromosomal coordinates chr9: 133729491-133729531; comprises, in the N- to C-terminus direction, an amino acid sequence encoded by exons 1-18 and exon 19, or a portion thereof, of a TSC1 gene, fused to an amino acid sequence encoded by exon 2, or a portion thereof, and exons 3-11 of an ABL1 gene; and/or is encoded by an ABL1 fusion nucleic acid molecule comprising, in 5′ to 3′ direction, exons 1-18 and exon 19, or a portion thereof, of a TSC1 gene, fused to exon 2, or a portion thereof, and exons 3-11 of an ABL1 gene. In some embodiments, the TSC1-ABL1 fusion polypeptide comprises the ABL1 kinase domain, or a portion of the ABL1 kinase domain having kinase activity.

In some embodiments, an ABL1 fusion polypeptide of the disclosure is a TSPAN18-ABL1 fusion polypeptide, wherein the TSPAN18-ABL1 fusion polypeptide: is encoded by an ABL1 fusion nucleic acid molecule comprising, in 5′ to 3′ direction, at least a portion of a TSPAN18 gene fused to at least a portion of an ABL1 gene; comprises a fusion between an amino acid sequence encoded by exon 2, or a portion thereof, of a TSPAN18 gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr11: 44881929-44881969 and/or a 3′ breakpoint within chromosomal coordinates chr9: 133729517-133729557; comprises, in the N- to C-terminus direction, an amino acid sequence encoded by exon 1 and exon 2, or a portion thereof, of a TSPAN18 gene, fused to an amino acid sequence encoded by exon 2, or a portion thereof, and exons 3-11 of an ABL1 gene; and/or is encoded by an ABL1 fusion nucleic acid molecule comprising, in 5′ to 3′ direction, exon 1 and exon 2, or a portion thereof, of a TSPAN18 gene, fused to exon 2, or a portion thereof, and exons 3-11 of an ABL1 gene. In some embodiments, the TSPAN18-ABL1 fusion polypeptide comprises the ABL1 kinase domain, or a portion of the ABL1 kinase domain having kinase activity.

In some embodiments, an ABL1 fusion polypeptide of the disclosure is a ZNF804B-ABL1 fusion polypeptide, wherein the ZNF804B-ABL1 fusion polypeptide: is encoded by an ABL1 fusion nucleic acid molecule comprising, in 5′ to 3′ direction, at least a portion of a ZNF804B gene fused to at least a portion of an ABL1 gene; is encoded by an ABL1 fusion nucleic acid molecule comprising or resulting from a breakpoint within intron 1 of a ZNF804B gene and/or a breakpoint within intron 1 of an ABL1 gene; comprises a fusion between an amino acid sequence encoded by exon 1, or a portion thereof, of a ZNF804B gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr7: 88521265-88521397 and/or a 3′ breakpoint within chromosomal coordinates chr9: 133729414-133729519; comprises, in the N- to C-terminus direction, an amino acid sequence encoded by exon 1, or a portion thereof, of a ZNF804B gene, fused to an amino acid sequence encoded by exon 2, or a portion thereof, and exons 3-11 of an ABL1 gene; and/or is encoded by an ABL1 fusion nucleic acid molecule comprising, in 5′ to 3′ direction, exon 1, or a portion thereof, of a ZNF804B gene, fused to exon 2, or a portion thereof, and exons 3-11 of an ABL1 gene. In some embodiments, the ZNF804B-ABL1 fusion polypeptide comprises the ABL1 kinase domain, or a portion of the ABL1 kinase domain having kinase activity.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 13 of a BCR gene and/or a breakpoint within intron 1 of an ABL1 gene; comprises a fusion between an amino acid sequence encoded by exon 13, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23631687-23632492 and/or a 3′ breakpoint within chromosomal coordinates chr9: 133604425-133604577; comprises, in the N- to C-terminus direction, an amino acid sequence encoded by exons 1-12 and exon 13, or a portion thereof, of a BCR gene, fused to an amino acid sequence encoded by exon 2, or a portion thereof, and exons 3-11 of an ABL1 gene; and/or is encoded by an ABL1 fusion nucleic acid molecule comprising, in 5′ to 3′ direction, exons 1-12 and exon 13, or a portion thereof, of a BCR gene, fused to exon 2, or a portion thereof, and exons 3-11 of an ABL1 gene. In some embodiments, the BCR-ABL1 fusion polypeptide comprises the ABL1 kinase domain, or a portion of the ABL1 kinase domain having kinase activity.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23632288-23632656 and/or a 3′ breakpoint within chromosomal coordinates chr9: 133663291-133663682; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; and/or is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene. In some embodiments, the BCR-ABL1 fusion polypeptide comprises the ABL1 kinase domain, or a portion of the ABL1 kinase domain having kinase activity.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23634171-23634647 and/or a 3′ breakpoint within chromosomal coordinates chr9: 133638886-133639512; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; and/or is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene. In some embodiments, the BCR-ABL1 fusion polypeptide comprises the ABL1 kinase domain, or a portion of the ABL1 kinase domain having kinase activity.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23633910-23634678 and/or a 3′ breakpoint within chromosomal coordinates chr9: 133724778-133725351; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; and/or is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene. In some embodiments, the BCR-ABL1 fusion polypeptide comprises the ABL1 kinase domain, or a portion of the ABL1 kinase domain having kinase activity.

In some embodiments, an ABL1 fusion polypeptide of the disclosure is an ABL1-NUP214 fusion polypeptide, wherein the ABL1-NUP214 fusion polypeptide: is encoded by an ABL1 fusion nucleic acid molecule comprising, in 5′ to 3′ direction, at least a portion of an ABL1 gene fused to at least a portion of a NUP214 gene; comprises a fusion between an amino acid sequence encoded by exon 1, or a portion thereof, of an ABL1 gene and an amino acid sequence encoded by exon 31, or a portion thereof, of a NUP214 gene; is encoded by an ABL1 fusion nucleic acid molecule comprising or resulting from a 5′ breakpoint within chromosomal coordinates chr9: 134090583-134090623 and/or a 3′ breakpoint within chromosomal coordinates chr9: 133710830-133710870; comprises, in the N- to C-terminus direction, an amino acid sequence encoded by exon 1, or a portion thereof, of an ABL1 gene, fused to an amino acid sequence encoded by exon 31, or a portion thereof, and exons 32-36 of a NUP214 gene; and/or is encoded by an ABL1 fusion nucleic acid molecule comprising, in 5′ to 3′ direction, exon 1, or a portion thereof, of an ABL1 gene, fused to exon 31, or a portion thereof, and exons 32-36 of a NUP214 gene.

In some embodiments, an ABL1 fusion polypeptide of the disclosure is an AIF1L-ABL1 fusion polypeptide, wherein the AIF1L-ABL1 fusion polypeptide: is encoded by an ABL1 fusion nucleic acid molecule comprising, in 5′ to 3′ direction, at least a portion of an AIF1L gene fused to at least a portion of an ABL1 gene; is encoded by an ABL1 fusion nucleic acid molecule comprising or resulting from a breakpoint within intron 2 of an AIF1L gene and/or a breakpoint within intron 1 of an ABL1 gene; comprises a fusion between an amino acid sequence encoded by exon 2, or a portion thereof, of an AIF1L gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr9: 133978032-133978137 and/or a 3′ breakpoint within chromosomal coordinates chr9: 133729409-133729618; comprises, in the N- to C-terminus direction, an amino acid sequence encoded by exon 1 and exon 2, or a portion thereof, of an AIF1L gene, fused to an amino acid sequence encoded by exon 2, or a portion thereof, and exons 3-11 of an ABL1 gene; and/or is encoded by an ABL1-fusion nucleic acid molecule comprising, in the 5′ to 3′ direction, exon 1 and exon 2, or a portion thereof, of an AIF1L gene, fused to exon 2, or a portion thereof, and exons 3-11 of an ABL1 gene. In some embodiments, the AIF1L-ABL1 fusion polypeptide comprises the ABL1 kinase domain, or a portion of the ABL1 kinase domain having kinase activity.

In some embodiments, an ABL1 fusion polypeptide of the disclosure is an EXOSC2-ABL1 fusion polypeptide, wherein the EXOSC2-ABL1 fusion polypeptide: is encoded by an ABL1 fusion nucleic acid molecule comprising, in 5′ to 3′ direction, at least a portion of an EXOSC2 gene fused to at least a portion of an ABL1 gene; is encoded by an ABL1 fusion nucleic acid molecule comprising or resulting from a breakpoint within exon 9 of an EXOSC2 gene and/or a breakpoint within exon 1 of an ABL1 gene; comprises a fusion between an amino acid sequence encoded by exon 9, or a portion thereof, of an EXOSC2 gene and an amino acid sequence encoded by exon 1, or a portion thereof, of an ABL1 gene; is encoded by an ABL1 fusion nucleic acid molecule comprising or resulting from a 5′ breakpoint within chromosomal coordinates chr9: 133579154-133579531 and/or a 3′ breakpoint within chromosomal coordinates chr9: 133589786-133589985; comprises, in the N- to C-terminus direction, an amino acid sequence encoded by exons 1-8 and exon 9, or a portion thereof, of an EXOSC2 gene, fused to an amino acid sequence encoded by exon 1, or a portion thereof, and exons 2-11 of an ABL1 gene; and/or is encoded by an ABL1 fusion nucleic acid molecule comprising, in 5′ to 3′ direction, exons 1-8 and exon 9, or a portion thereof, of an EXOSC2 gene, fused to exon 1, or a portion thereof, and exons 2-11 of an ABL1 gene. In some embodiments, the EXOSC2-ABL1 fusion polypeptide comprises the ABL1 kinase domain, or a portion of the ABL1 kinase domain having kinase activity.

In some embodiments, an ABL1 fusion polypeptide of the disclosure is a NUP214-ABL1 fusion polypeptide, wherein the NUP214-ABL1 fusion polypeptide: is encoded by an ABL1 fusion nucleic acid molecule comprising, in 5′ to 3′ direction, at least a portion of a NUP214 gene fused to at least a portion of an ABL1 gene; is encoded by an ABL1 fusion nucleic acid molecule comprising or resulting from a breakpoint within intron 34 of a NUP214 gene and/or a breakpoint within exon 3 of an ABL1 gene; comprises a fusion between an amino acid sequence encoded by exon 34, or a portion thereof, of a NUP214 gene and an amino acid sequence encoded by exon 3, or a portion thereof, of an ABL1 gene; is encoded by an ABL1 fusion nucleic acid molecule comprising or resulting from a 5′ breakpoint within chromosomal coordinates chr9: 134106080-134106120 and/or a 3′ breakpoint within chromosomal coordinates chr9: 133730195-133730235; comprises, in the N- to C-terminus direction, an amino acid sequence encoded by exons 1-33 and exon 34, or a portion thereof, of a NUP214 gene, fused to an amino acid sequence encoded by exon 3, or a portion thereof, and exons 4-11 of an ABL1 gene; and/or is encoded by an ABL1 fusion nucleic acid molecule comprising, in 5′ to 3′ direction, exons 1-33 and exon 34, or a portion thereof, of a NUP214 gene, fused to exon 3, or a portion thereof, and exons 4-11 of an ABL1 gene. In some embodiments, the NUP214-ABL1 fusion polypeptide comprises the ABL1 kinase domain, or a portion of the ABL1 kinase domain having kinase activity.

In some embodiments, an ABL1 fusion polypeptide of the disclosure is a NUP214-ABL1 fusion polypeptide, wherein the NUP214-ABL1 fusion polypeptide: is encoded by an ABL1 fusion nucleic acid molecule comprising, in 5′ to 3′ direction, at least a portion of a NUP214 gene fused to at least a portion of an ABL1 gene; comprises a fusion between an amino acid sequence encoded by exon 34, or a portion thereof, of a NUP214 gene and an amino acid sequence encoded by exon 3, or a portion thereof, of an ABL1 gene; is encoded by an ABL1 fusion nucleic acid molecule comprising or resulting from a 5′ breakpoint within chromosomal coordinates chr9: 134106030-134106070 and/or a 3′ breakpoint within chromosomal coordinates chr9: 133730218-133730258; comprises, in the N- to C-terminus direction, an amino acid sequence encoded by exons 1-33 and exon 34, or a portion thereof, of a NUP214 gene, fused to an amino acid sequence encoded by exon 3, or a portion thereof, and exons 4-11 of an ABL1 gene; and/or is encoded by an ABL1 fusion nucleic acid molecule comprising, in 5′ to 3′ direction, exons 1-33 and exon 34, or a portion thereof, of a NUP214 gene, fused to exon 3, or a portion thereof, and exons 4-11 of an ABL1 gene. In some embodiments, the NUP214-ABL1 fusion polypeptide comprises the ABL1 kinase domain, or a portion of the ABL1 kinase domain having kinase activity.

In some embodiments, an ABL1 fusion polypeptide of the disclosure is a NUP214-ABL1 fusion polypeptide, wherein the NUP214-ABL1 fusion polypeptide: is encoded by an ABL1 fusion nucleic acid molecule comprising, in 5′ to 3′ direction, at least a portion of a NUP214 gene fused to at least a portion of an ABL1 gene; comprises a fusion between an amino acid sequence encoded by exon 34, or a portion thereof, of a NUP214 gene and an amino acid sequence encoded by exon 3, or a portion thereof, of an ABL1 gene; is encoded by an ABL1 fusion nucleic acid molecule comprising or resulting from a 5′ breakpoint within chromosomal coordinates chr9: 134106046-134106086 and/or a 3′ breakpoint within chromosomal coordinates chr9: 133730203-133730243; comprises, in the N- to C-terminus direction, an amino acid sequence encoded by exons 1-33 and exon 34, or a portion thereof, of a NUP214 gene, fused to an amino acid sequence encoded by exon 3, or a portion thereof, and exons 4-11 of an ABL1 gene; and/or is encoded by an ABL1 fusion nucleic acid molecule comprising, in 5′ to 3′ direction, exons 1-33 and exon 34, or a portion thereof, of a NUP214 gene, fused to exon 3, or a portion thereof, and exons 4-11 of an ABL1 gene. In some embodiments, the NUP214-ABL1 fusion polypeptide comprises the ABL1 kinase domain, or a portion of the ABL1 kinase domain having kinase activity.

In some embodiments, an ABL1 fusion polypeptide of the disclosure is a NUP214-ABL1 fusion polypeptide, wherein the NUP214-ABL1 fusion polypeptide: is encoded by an ABL1 fusion nucleic acid molecule comprising, in 5′ to 3′ direction, at least a portion of a NUP214 gene fused to at least a portion of an ABL1 gene; comprises a fusion between an amino acid sequence encoded by exon 34, or a portion thereof, of a NUP214 gene and an amino acid sequence encoded by exon 3, or a portion thereof, of an ABL1 gene; is encoded by an ABL1 fusion nucleic acid molecule comprising or resulting from a 5′ breakpoint within chromosomal coordinates chr9: 134106028-134106068 and/or a 3′ breakpoint within chromosomal coordinates chr9: 133730234-133730274; comprises, in the N- to C-terminus direction, an amino acid sequence encoded by exons 1-33 and exon 34, or a portion thereof, of a NUP214 gene, fused to an amino acid sequence encoded by exon 3, or a portion thereof, and exons 4-11 of an ABL1 gene; and/or is encoded by an ABL1 fusion nucleic acid molecule comprising, in 5′ to 3′ direction, exons 1-33 and exon 34, or a portion thereof, of a NUP214 gene, fused to exon 3, or a portion thereof, and exons 4-11 of an ABL1 gene. In some embodiments, the NUP214-ABL1 fusion polypeptide comprises the ABL1 kinase domain, or a portion of the ABL1 kinase domain having kinase activity.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; comprises a fusion between an amino acid sequence encoded by exon 1, or a portion thereof, of a BCR gene and an amino acid sequence encoded by exon 3, or a portion thereof, of an ABL1 gene; is encoded by an ABL1 fusion nucleic acid molecule comprising or resulting from a 5′ breakpoint within chromosomal coordinates chr22: 23524272-23524312 and/or a 3′ breakpoint within chromosomal coordinates chr9: 133730218-133730258; comprises, in the N- to C-terminus direction, an amino acid sequence encoded by exon 1, or a portion thereof, of a BCR gene, fused to an amino acid sequence encoded by exon 3, or a portion thereof, and exons 4-11 of an ABL1 gene; and/or is encoded by an ABL1 fusion nucleic acid molecule comprising, in 5′ to 3′ direction, exon 1, or a portion thereof, of a BCR gene, fused to exon 3, or a portion thereof, and exons 4-11 of an ABL1 gene. In some embodiments, the BCR-ABL1 fusion polypeptide comprises the ABL1 kinase domain, or a portion of the ABL1 kinase domain having kinase activity.

In some embodiments, an ABL1 fusion polypeptide of the disclosure is an SNX2-ABL1 fusion polypeptide, wherein the SNX2-ABL1 fusion polypeptide: is encoded by an ABL1 fusion nucleic acid molecule comprising, in 5′ to 3′ direction, at least a portion of an SNX2 gene fused to at least a portion of an ABL1 gene; comprises a fusion between an amino acid sequence encoded by exon 3, or a portion thereof, of an SNX2 gene and an amino acid sequence encoded by exon 4, or a portion thereof, of an ABL1 gene; is encoded by an ABL1 fusion nucleic acid molecule comprising or resulting from a 5′ breakpoint within chromosomal coordinates chr5: 122135451-122135491 and/or a 3′ breakpoint within chromosomal coordinates chr9: 133738222-133738262; comprises, in the N- to C-terminus direction, an amino acid sequence encoded by exons 1-2 and exon 3, or a portion thereof, of an SNX2 gene, fused to an amino acid sequence encoded by exon 4, or a portion thereof, and exons 5-11 of an ABL1 gene; and/or is encoded by an ABL1 fusion nucleic acid molecule comprising, in the 5′ to 3′ direction, exons 1-2 and exon 3, or a portion thereof, of an SNX2 gene, fused to exon 4, or a portion thereof, and exons 5-11 of an ABL1 gene. In some embodiments, the SNX2-ABL1 fusion polypeptide comprises the ABL1 kinase domain, or a portion of the ABL1 kinase domain having kinase activity.

In some embodiments, an ABL1 fusion polypeptide of the disclosure is an SNX2-ABL1 fusion polypeptide, wherein the SNX2-ABL1 fusion polypeptide: is encoded by an ABL1 fusion nucleic acid molecule comprising, in 5′ to 3′ direction, at least a portion of an SNX2 gene fused to at least a portion of an ABL1 gene; comprises a fusion between an amino acid sequence encoded by exon 3, or a portion thereof, of an SNX2 gene and an amino acid sequence encoded by exon 4, or a portion thereof, of an ABL1 gene; is encoded by an ABL1 fusion nucleic acid molecule comprising or resulting from a 5′ breakpoint within chromosomal coordinates chr5: 122135445-122135485 and/or a 3′ breakpoint within chromosomal coordinates chr9: 133738214-133738254; comprises, in the N- to C-terminus direction, an amino acid sequence encoded by exons 1-2 and exon 3, or a portion thereof, of an SNX2 gene, fused to an amino acid sequence encoded by exon 4, or a portion thereof, and exons 5-11 of an ABL1 gene; and/or is encoded by an ABL1 fusion nucleic acid molecule comprising, in the 5′ to 3′ direction, exons 1-2 and exon 3, or a portion thereof, of an SNX2 gene, fused to exon 4, or a portion thereof, and exons 5-11 of an ABL1 gene. In some embodiments, the SNX2-ABL1 fusion polypeptide comprises the ABL1 kinase domain, or a portion of the ABL1 kinase domain having kinase activity.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 12 of a BCR gene and/or a breakpoint within intron 1 of an ABL1 gene; comprises a fusion between an amino acid sequence encoded by exon 12, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23631693-23632188 and/or a 3′ breakpoint within chromosomal coordinates chr9: 133607683-133608245; comprises, in the N- to C-terminus direction, an amino acid sequence encoded by exons 1-11 and exon 12, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-11 and exon 12, or a portion thereof, of a BCR gene, fused to exon 2, or a portion thereof, and exons 3-11 of an ABL1 gene; comprises the amino acid sequence of SEQ ID NO: 31, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 31; and/or is encoded by the nucleotide sequence of SEQ ID NO: 10, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 10. In some embodiments, the BCR-ABL1 fusion polypeptide comprises the ABL1 kinase domain, or a portion of the ABL1 kinase domain having kinase activity.

In some embodiments, an ABL1 fusion polypeptide of the disclosure is a BMP2K-ABL1 fusion polypeptide, wherein the BMP2K-ABL1 fusion polypeptide: is encoded by an ABL1 fusion nucleic acid molecule comprising in 5′ to 3′ direction, at least a portion of a BMP2K gene fused to at least a portion of an ABL1 gene; comprises a fusion between an amino acid sequence encoded by exon 15, or a portion thereof, of a BMP2K gene and an amino acid sequence encoded by exon 3, or a portion thereof, of an ABL1 gene; is encoded by an ABL1 fusion nucleic acid molecule comprising or resulting from a 5′ breakpoint within chromosomal coordinates chr4: 79800022-79800062 and/or a 3′ breakpoint within chromosomal coordinates chr9: 133730205-133730245; comprises, in the N- to C-terminus direction, an amino acid sequence encoded by exons 1-14 and exon 15, or a portion thereof, of a BMP2K gene, fused to an amino acid sequence encoded by exon 3, or a portion thereof, and exons 4-11 of an ABL1 gene; and/or is encoded by an ABL1 fusion nucleic acid molecule comprising, in 5′ to 3′ direction, exons 1-14 and exon 15, or a portion thereof, of a BMP2K gene, fused to exon 3, or a portion thereof, and exons 4-11 of an ABL1 gene. In some embodiments, the BMP2K-ABL1 fusion polypeptide comprises the ABL1 kinase domain, or a portion of the ABL1 kinase domain having kinase activity.

In some embodiments, an ABL1 fusion polypeptide of the disclosure comprises a fusion between an ABL1 polypeptide, or a portion thereof, fused to a polypeptide encoded by a second gene, or a portion thereof, wherein the ABL1 fusion polypeptide results from an ABL1 breakpoint within exon 1 of an ABL1 gene. In some embodiments, the ABL1 fusion polypeptide comprises, in the N- to C-terminus direction, an amino acid sequence encoded by the second gene, or a portion thereof, fused to an amino acid sequence encoded by exon 1 of an ABL1 gene, or a portion thereof. In some embodiments, the ABL1 fusion polypeptide comprises a fusion between an amino acid sequence encoded by an exon, or a portion thereof, of the second gene fused to an amino acid sequence encoded by exon 1, or a portion thereof, of an ABL1 gene. In some embodiments, the ABL1 fusion polypeptide results from a breakpoint within chromosomal coordinates chr9: 133589786-133589985. In some embodiments, the ABL1 fusion polypeptide comprises, in the N- to C-terminus direction, an amino acid sequence encoded by one or more exons, or portions thereof, of the second gene, fused to an amino acid sequence encoded by exon 1, or a portion thereof, and exons 2-11 of ABL1. In some embodiments, the second gene is an EXOSC2 gene, and wherein the ABL1 fusion polypeptide: comprises a fusion between an amino acid sequence encoded by exon 9, or a portion thereof, of an EXOSC2 gene fused to an amino acid sequence encoded by exon 1, or a portion thereof, of an ABL1 gene; results from a breakpoint within exon 9 of an EXOSC2 gene and a breakpoint within exon 1 of an ABL1 gene; results from a 5′ breakpoint within chromosomal coordinates chr9: 133579154-133579531 and/or a 3′ breakpoint within chromosomal coordinates chr9: 133589786-133589985; and/or comprises, in the N- to C-terminus direction, an amino acid sequence encoded by exons 1-8 and exon 9, or a portion thereof, of an EXOSC2 gene, fused to an amino acid sequence encoded by exon 1, or a portion thereof, and exons 2-11 of an ABL1 gene.

In some embodiments, an ABL1 fusion polypeptide of the disclosure comprises a fusion between an ABL1 polypeptide, or a portion thereof, fused to a polypeptide encoded by a second gene, or a portion thereof, wherein the ABL1 fusion polypeptide results from an ABL1 breakpoint within exon 2 of ABL1. In some embodiments, the ABL1 fusion polypeptide comprises, in the N- to C-terminus direction, an amino acid sequence encoded by the second gene, or a portion thereof, fused to an amino acid sequence encoded by exon 2, or a portion thereof, of an ABL1 gene. In some embodiments, the ABL1 fusion polypeptide comprises a fusion between an amino acid sequence encoded by an exon, or a portion thereof, of the second gene fused to an amino acid sequence encoded by exon 2, or a portion thereof, of an ABL1 gene. In some embodiments, the ABL1 fusion polypeptide results from a breakpoint within chromosomal coordinates chr9: 133729584-133729766. In some embodiments, the ABL1 fusion polypeptide comprises, in the N- to C-terminus direction, an amino acid sequence encoded by one or more exons, or portions thereof, of the second gene, fused to an amino acid sequence encoded by exon 2, or a portion thereof, and exons 3-11 of an ABL1 gene. In some embodiments, the second gene is an APOOL gene, wherein the ABL1 fusion polypeptide: results from a breakpoint within intron 2 of an APOOL gene, and exon 2 of an ABL1 gene; comprises a fusion between an amino acid sequence encoded by exon 2, or a portion thereof, of an APOOL gene fused to an amino acid sequence encoded by exon 2, or a portion thereof, of an ABL1 gene; results from a 5′ breakpoint within chromosomal coordinates chrX: 84302705-84302904 and/or 3′ breakpoint within chromosomal coordinates chr9: 133729584-133729766; and/or comprises, in the N- to C-terminus direction, an amino acid sequence encoded by exon 1 and exon 2, or a portion thereof of an APOOL gene, fused to an amino acid sequence encoded by exon 2, or a portion thereof, and exons 3-11 of an ABL1 gene.

In some embodiments, an ABL1 fusion polypeptide of the disclosure comprises a fusion between an ABL1 polypeptide, or a portion thereof, fused to a polypeptide encoded by a second gene, or a portion thereof, wherein the ABL1 fusion polypeptide results from an ABL1 breakpoint within exon 3 of an ABL1 gene. In some embodiments, the ABL1 fusion polypeptide comprises, in the N- to C-terminus direction, an amino acid sequence encoded by the second gene, or a portion thereof, fused to an amino acid sequence encoded by exon 3, or a portion thereof, of an ABL1 gene. In some embodiments, the ABL1 fusion polypeptide comprises a fusion between an amino acid sequence encoded by an exon, or a portion thereof, of the second gene fused to an amino acid sequence encoded by exon 3, or a portion thereof, of an ABL1 gene. In some embodiments, the ABL1 fusion polypeptide results from a breakpoint within chromosomal coordinates chr9: 133730205-133730245, chr9: 133730195-133730235, chr9: 133730203-133730243, chr9: 133730234-133730274 or chr9: 133730218-133730258. In some embodiments, the ABL1 fusion polypeptide comprises, in the N- to C-terminus direction, an amino acid sequence encoded by one or more exons, or portions thereof, of the second gene, fused to an amino acid sequence encoded by exon 3, or a portion thereof, and exons 4-11 of an ABL1 gene. In some embodiments, the second gene is a NUP214 gene, wherein the ABL1 fusion polypeptide: comprises a fusion between an amino acid sequence encoded by exon 34, or a portion thereof, of a NUP214 gene fused to an amino acid sequence encoded by exon 3, or a portion thereof, of an ABL1 gene; results from a breakpoint within intron 34 of a NUP214 gene and a breakpoint within exon 3 of an ABL1 gene; results from: a 5′ breakpoint within chromosomal coordinates chr9: 134106080-134106120 and/or a 3′ breakpoint within chromosomal coordinates chr9: 133730195-133730235, a 5′ breakpoint within chromosomal coordinates chr9: 134106030-134106070 and/or a 3′ breakpoint within chromosomal coordinates chr9: 133730218-133730258, a 5′ breakpoint within chromosomal coordinates chr9: 134106046-134106086 and/or a 3′ breakpoint within chromosomal coordinates chr9: 133730203-133730243, or a 5′ breakpoint within chromosomal coordinates chr9: 134106028-134106068 and/or a 3′ breakpoint within chromosomal coordinates chr9: 133730234-133730274; and/or comprises, in the N- to C-terminus direction, an amino acid sequence encoded by exons 1-33 and exon 34, or a portion thereof, of a NUP214 gene, fused to an amino acid sequence encoded by exon 3, or a portion thereof, and exons 4-11 of an ABL1 gene. In some embodiments, the second gene is a BMP2K gene, wherein the ABL1 fusion polypeptide: comprises a fusion between an amino acid sequence encoded by exon 15, or a portion thereof, of a BMP2K gene fused to an amino acid sequence encoded by exon 3, or a portion thereof, of an ABL1 gene; results from a 5′ breakpoint within chromosomal coordinates chr4: 79800022-79800062 and/or a 3′ breakpoint within chromosomal coordinates chr9: 133730205-133730245; and/or comprises, in the N- to C-terminus direction, an amino acid sequence encoded by exons 1-14 and exon 15, or a portion thereof, of a BMP2K gene, fused to an amino acid sequence encoded by exon 3, or a portion thereof, and exons 4-11 of an ABL1 gene. In some embodiments, the second gene is a BCR gene, wherein the ABL1 fusion polypeptide: comprises a fusion between an amino acid sequence encoded by exon 1, or a portion thereof, of a BCR gene fused to an amino acid sequence encoded by exon 3, or a portion thereof, of an ABL1 gene; results from a 5′ breakpoint within chromosomal coordinates chr22: 23524272-23524312 and/or a 3′ breakpoint within chromosomal coordinates chr9: 133730218-133730258; and/or comprises, in the N- to C-terminus direction, an amino acid sequence encoded by exon 1, or a portion thereof, of a BCR gene, fused to an amino acid sequence encoded by exon 3, or a portion thereof, and exons 4-11 of an ABL1 gene.

In some embodiments, an ABL1 fusion polypeptide of the disclosure comprises a fusion between an ABL1 polypeptide, or a portion thereof, fused to a polypeptide encoded by a second gene, or a portion thereof, wherein the ABL1 fusion polypeptide comprises or results from an ABL1 breakpoint within exon 4 of ABL1. In some embodiments, the ABL1 fusion polypeptide comprises, in the N- to C-terminus direction, an amino acid sequence encoded by the second gene, or a portion thereof, fused to an amino acid sequence encoded by exon 4, or a portion thereof, of an ABL1 gene. In some embodiments, the ABL1 fusion polypeptide comprises a fusion between an amino acid sequence encoded by an exon, or a portion thereof, of the second gene fused to an amino acid sequence encoded by exon 4, or a portion thereof, of an ABL1 gene. In some embodiments, the ABL1 fusion polypeptide results from a breakpoint within chromosomal coordinates chr9: 133738173-133738213, chr9: 133738222-133738262, or chr9: 133738214-133738254. In some embodiments, the ABL1 fusion polypeptide comprises, in the N- to C-terminus direction, an amino acid sequence encoded by one or more exons, or portions thereof, of the second gene, fused to an amino acid sequence encoded by exon 4, or a portion thereof, and exons 5-11 of ABL1. In some embodiments, the second gene is an RCSD1 gene, and wherein the ABL1 fusion polypeptide: comprises a fusion between an amino acid sequence encoded by exon 4, or a portion thereof, of an RCSD1 gene fused to an amino acid sequence encoded by exon 4, or a portion thereof, of an ABL1 gene; results from a 5′ breakpoint within chromosomal coordinates chr1: 167654668-167654708 and/or a 3′ breakpoint within chromosomal coordinates chr9: 133738173-133738213; comprises, in the N- to C-terminus direction, an amino acid sequence encoded by exons 1-3 and exon 4, or a portion thereof, of an RCSD1 gene, fused to an amino acid sequence encoded by exon 4, or a portion thereof, and exons 5-11 of an ABL1 gene; is encoded by the nucleotide sequence of SEQ ID NO: 21, or a nucleotide sequence having at least about 70% homology thereto; and/or comprises the amino acid sequence of SEQ ID NO: 42, or an amino acid sequence having at least about 70% homology thereto. In some embodiments, the second gene is an SNX2 gene, and wherein the ABL1 fusion polypeptide: comprises a fusion between an amino acid sequence encoded by exon 3, or a portion thereof, of an SNX2 gene fused to an amino acid sequence encoded by exon 4, or a portion thereof, of an ABL1 gene; results from: a 5′ breakpoint within chromosomal coordinates chr5: 122135451-122135491 and/or a 3′ breakpoint within chromosomal coordinates chr9: 133738222-133738262, or a 5′ breakpoint within chromosomal coordinates chr5: 122135445-122135485 and/or a 3′ breakpoint within chromosomal coordinates chr9: 133738214-133738254; and/or comprises, in the N- to C-terminus direction, an amino acid sequence encoded by exons 1-2 and exon 3, or a portion thereof, of an SNX2 gene, fused to an amino acid sequence encoded by exon 4, or a portion thereof, and exons 5-11 of an ABL1 gene.

In some embodiments, an ABL1 fusion polypeptide of the disclosure comprises a fusion between an ABL1 polypeptide, or a portion thereof, fused to a polypeptide encoded by a second gene, or a portion thereof, wherein the ABL1 fusion polypeptide results from an ABL1 breakpoint within exon 10 of an ABL1 gene. In some embodiments, the ABL1 fusion polypeptide comprises, in the N- to C-terminus direction, an amino acid sequence encoded by exon 10, or a portion thereof, of an ABL1 gene fused to an amino acid sequence encoded by the second gene, or a portion thereof. In some embodiments, the ABL1 fusion polypeptide comprises a fusion between an amino acid sequence encoded by an exon, or a portion thereof, of the second gene fused to an amino acid sequence encoded by exon 10, or a portion thereof, of an ABL1 gene. In some embodiments, the ABL1 fusion polypeptide results from a breakpoint within chromosomal coordinates chr9: 133755953-133755993. In some embodiments, the ABL1 fusion polypeptide comprises, in the N- to C-terminus direction, an amino acid sequence encoded by exons 1-9 and exon 10, or a portion thereof, of an ABL1 gene fused to an amino acid sequence encoded by one or more exons, or portions thereof, of the second gene. In some embodiments, the second gene is a DNAH2 gene, wherein the ABL1 fusion polypeptide: comprises a fusion between an amino acid sequence encoded by exon 10, or a portion thereof, of an ABL1 gene fused to an amino acid sequence encoded by exon 4, or a portion thereof, of a DNAH2 gene; results from a 5′ breakpoint within chromosomal coordinates chr9: 133755953-133755993 and/or a 3′ breakpoint within chromosomal coordinates chr17: 7636399-7636439; and/or comprises, in the N- to C-terminus direction, an amino acid sequence encoded by exons 1-9 and exon 10, or a portion thereof, of an ABL1 gene fused to an amino acid sequence encoded by exon 4, or a portion thereof, and exons 5-85 of a DNAH2 gene.

In some embodiments, an ABL1 fusion polypeptide of the disclosure comprises a fusion between an ABL1 polypeptide, or a portion thereof, fused to a polypeptide encoded by a second gene, or a portion thereof, wherein the ABL1 fusion polypeptide results from an ABL1 breakpoint within intron 4 of an ABL1 gene. In some embodiments, the ABL1 fusion polypeptide comprises, in the N- to C-terminus direction, an amino acid sequence encoded by the second gene, or a portion thereof, fused to an amino acid sequence encoded by exon 5, or a portion thereof, of an ABL1 gene. In some embodiments, the ABL1 fusion polypeptide comprises a fusion between an amino acid sequence encoded by an exon of the second gene, or a portion thereof, fused to an amino acid sequence encoded by exon 5, or a portion thereof, of an ABL1 gene. In some embodiments, the ABL1 fusion polypeptide results from a breakpoint within chromosomal coordinates chr9: 133747364-133747762. In some embodiments, the ABL1 fusion polypeptide comprises, in the N- to C-terminus direction, an amino acid sequence encoded by one or more exons, or portions thereof, of the second gene, fused to an amino acid sequence encoded by exon 5, or a portion thereof, and exons 6-11 of an ABL1 gene. In some embodiments, the second gene is a MED27 gene, wherein the ABL1 fusion polypeptide: comprises a fusion between an amino acid sequence encoded by exon 4, or a portion thereof, of a MED27 gene fused to an amino acid sequence encoded by exon 5, or a portion thereof, of an ABL1 gene; results from a breakpoint within intron 4 of a MED27 gene and a breakpoint within intron 4 of an ABL1 gene; results from a 5′ breakpoint within chromosomal coordinates chr9: 134774423-134776006 and/or a 3′ breakpoint within chromosomal coordinates chr9: 133747364-133747762; comprises, in the N- to C-terminus direction, an amino acid sequence encoded by exons 1-3 and exon 4, or a portion thereof, of a MED27 gene, fused to an amino acid sequence encoded by exon 5, or a portion thereof, and exons 6-11 of an ABL1 gene; is encoded by the nucleotide sequence of SEQ ID NO: 3, or a nucleotide sequence having at least about 70% homology thereto; and/or comprises the amino acid sequence of SEQ ID NO: 24, or an amino acid sequence having at least about 70% homology thereto.

In some embodiments, an ABL1 fusion polypeptide of the disclosure comprises a fusion between an ABL1 polypeptide, or a portion thereof, fused to a polypeptide encoded by a second gene, or a portion thereof, wherein the ABL1 fusion polypeptide results from an ABL1 breakpoint within intron 5 of ABL1. In some embodiments, the ABL1 fusion polypeptide comprises, in the N- to C-terminus direction, an amino acid sequence encoded by the second gene, or a portion thereof, fused to an amino acid sequence encoded by exon 6, or a portion thereof, of an ABL1 gene. In some embodiments, the ABL1 fusion polypeptide comprises a fusion between an amino acid sequence encoded by an exon, or a portion thereof, of the second gene fused to an amino acid sequence encoded by exon 6, or a portion thereof, of an ABL1 gene. In some embodiments, the ABL1 fusion polypeptide results from a breakpoint within chromosomal coordinates chr9: 133748211-133748501. In some embodiments, the ABL1 fusion polypeptide comprises, in the N- to C-terminus direction, an amino acid sequence encoded by one or more exons, or portions thereof, of the second gene, fused to an amino acid sequence encoded by exon 6, or a portion thereof, and exons 7-11 of an ABL1 gene. In some embodiments, the second gene is an SLC27A4 gene, wherein the ABL1 fusion polypeptide: comprises a fusion between an amino acid sequence encoded by exon 12, or a portion thereof, of a SLC27A4 gene fused to an amino acid sequence encoded by exon 6, or a portion thereof, of an ABL1 gene; results from a breakpoint within intron 12 of a SLC27A4 gene and a breakpoint within intron 5 of an ABL1 gene; results from a 5′ breakpoint within chromosomal coordinates chr9: 131119603-131119801 and/or a 3′ breakpoint within chromosomal coordinates chr9: 133748211-133748501; comprises, in the N- to C-terminus direction, an amino acid sequence encoded by exons 1-11 and exon 12, or a portion thereof, of an SLC27A4 gene, fused to an amino acid sequence encoded by exon 6, or a portion thereof, and exons 7-11 of an ABL1 gene; is encoded by the nucleotide sequence of SEQ ID NO: 5, or a nucleotide sequence having at least about 70% homology thereto; and/or comprises the amino acid sequence of SEQ ID NO: 26, or an amino acid sequence having at least about 70% homology thereto.

(iii) Cancers and Methods Related Thereto

Certain aspects of the present disclosure relate to methods for identifying an individual having a cancer who may benefit from a treatment comprising an anti-cancer therapy; selecting a treatment for an individual having a cancer; identifying one or more treatment options for an individual having a cancer; predicting survival of an individual having a cancer; treating or delaying progression of cancer; monitoring, evaluating or screening an individual having a cancer; assessing an ABL1 fusion nucleic acid molecule or an ABL1 fusion polypeptide in a cancer in an individual; detecting the presence or absence of a cancer in an individual; monitoring progression or recurrence of a cancer in an individual; or identifying a candidate treatment for a cancer in an individual in need thereof.

In some embodiments, of any of the methods provided herein, the methods comprise acquiring knowledge of or detecting in a sample from an individual having a cancer an ABL1 fusion nucleic acid molecule of the disclosure, e.g., any of the ABL1-fusion nucleic acid molecules described above and/or in the Examples herein. In other embodiments, the methods comprise acquiring knowledge of or detecting in a sample from an individual having a cancer an ABL1 fusion polypeptide of the disclosure, e.g., any of the ABL1-fusion polypeptides described above and/or in the Examples herein.

In some embodiments of any of the methods provided herein, detection of an ABL1 fusion nucleic acid molecule or an ABL1 fusion polypeptide of the disclosure in the sample identifies the individual as one who may benefit from a treatment comprising the anti-cancer therapy, such as an anti-cancer therapy provided herein, e.g., an ABL1-targeted therapy.

In some embodiments, the methods comprise detecting, in a first sample obtained from the individual at a first time point, the presence or absence of an ABL1 fusion nucleic acid molecule or an ABL1 fusion polypeptide of the disclosure. In some embodiments, the methods further comprise detecting, in a second sample obtained from the individual at a second time point after the first time point, the presence or absence of an ABL1 fusion nucleic acid molecule or an ABL1 fusion polypeptide of the disclosure. In some embodiments, the methods further comprise providing an assessment of cancer progression or cancer recurrence in the individual based, at least in part, on the presence or absence of the ABL1 fusion nucleic acid molecule or ABL1 fusion polypeptide in the first sample and/or in the second sample. In some embodiments, the presence of the ABL1 fusion nucleic acid molecule or ABL1 fusion polypeptide in the first sample and/or in the second sample identifies the individual as having increased risk of cancer progression or cancer recurrence. In some embodiments, the methods further comprise selecting a treatment, administering a treatment, adjusting a treatment, adjusting the dose of a treatment, or applying a treatment to the individual based, at least in part, on detecting the presence of the ABL1 fusion nucleic acid molecule or ABL1 fusion polypeptide in the first sample and/or in the second sample, wherein the treatment comprises an anti-cancer therapy, such as an anti-cancer therapy provided herein, e.g., and ABL1-targeted therapy.

In some embodiments, the methods comprise performing DNA sequencing on a sample obtained from the individual to determine a sequencing mutation profile on a group of genes, wherein the sequencing mutation profile identifies the presence or absence of an ABL1 fusion nucleic acid molecule of the disclosure. In some embodiments, the methods further comprise identifying a candidate treatment for a cancer in an individual, based at least in part on the sequencing mutation profile. In some embodiments, the candidate treatment comprises an anti-cancer therapy, such as an anti-cancer therapy provided herein, e.g., an ABL1-targeted therapy. In some embodiments, the sequencing mutation profile identifies the presence or absence of a fragment of the ABL1 fusion nucleic acid molecule comprising a breakpoint or fusion junction, e.g., one or more of the corresponding breakpoints described herein. In some embodiments, the presence of the ABL1 fusion nucleic acid molecule in the sample identifies the individual as one who may benefit from a treatment comprising an anti-cancer therapy, e.g., an anti-cancer therapy provided herein, such as an ABL1-targeted therapy. In some embodiments, the presence of the ABL1 fusion nucleic acid molecule in the sample predicts the individual to have longer survival when treated with a treatment comprising an anti-cancer therapy (e.g., an anti-cancer therapy provided herein, such as an ABL1-targeted therapy), as compared to survival of an individual whose cancer does not comprise an ABL1 fusion nucleic acid molecule.

In some embodiments of any of the methods provided herein, the methods further comprise generating a report comprising one or more treatment options identified for an individual (e.g., an individual having cancer) based at least in part on detection of an ABL1 fusion nucleic acid molecule or an ABL1 fusion polypeptide of the disclosure in a sample from the individual, wherein the one or more treatment options comprise an anti-cancer therapy, such as an anti-cancer therapy provided herein, e.g., an ABL1-targeted therapy.

In some embodiments of any of the methods provided herein, responsive to acquisition of knowledge of an ABL1 fusion nucleic acid molecule or an ABL1 fusion polypeptide of the disclosure in a sample from an individual (e.g., an individual having cancer): (i) the individual is classified as a candidate to receive a treatment comprising an anti-cancer therapy, such as an anti-cancer therapy provided herein, e.g., an ABL1-targeted therapy; and/or (ii) the individual is identified as likely to respond to a treatment that comprises an anti-cancer therapy, such as an anti-cancer therapy provided herein, e.g., an ABL1-targeted therapy. In some embodiments, responsive to acquisition of knowledge of an ABL1 fusion nucleic acid molecule or an ABL1 fusion polypeptide of the disclosure in a sample from an individual (e.g., an individual having cancer), the individual is predicted to have longer survival when treated with a treatment comprising an anti-cancer therapy, such as an anti-cancer therapy provided herein, e.g., an ABL1-targeted therapy, as compared to survival of an individual whose cancer does not comprise or exhibit an ABL1 fusion nucleic acid molecule or an ABL1 fusion polypeptide. In some embodiments, responsive to acquisition of knowledge of an ABL1 fusion nucleic acid molecule or an ABL1 fusion polypeptide of the disclosure in a sample from an individual (e.g., an individual having cancer), the individual is predicted to have acquired resistance to a prior anti-cancer therapy administered to the individual, the individual is predicted to respond to an anti-cancer therapy (e.g., an anti-cancer therapy provided herein, such as an ABL1-targeted therapy), and/or the individual is predicted to have poor prognosis, as compared to an individual whose cancer does not comprise the fusion nucleic acid molecule or polypeptide.

In some embodiments, responsive to acquisition of knowledge of an ABL1 fusion nucleic acid molecule or an ABL1 fusion polypeptide of the disclosure in a sample from an individual (e.g., an individual having cancer), the methods comprise administering to the individual an effective amount of a treatment that comprises an anti-cancer therapy, such as an anti-cancer therapy provided herein, e.g., an ABL1-targeted therapy.

In some embodiments of any of the methods provided herein, the methods further comprise generating a report comprising one or more treatment options identified for an individual (e.g., an individual having cancer) based at least in part on knowledge of the presence of an ABL1 fusion nucleic acid molecule or an ABL1 fusion polypeptide of the disclosure in a sample from the individual, wherein the one or more treatment options comprise an anti-cancer therapy, such as an anti-cancer therapy provided herein, e.g., an ABL1-targeted therapy.

In some embodiments, acquiring knowledge of an ABL1 fusion nucleic acid molecule or an ABL1 fusion polypeptide of the disclosure in a sample comprises detecting the fusion nucleic acid molecule or polypeptide in the sample.

In some embodiments of any of the methods provided herein, detecting an ABL1 fusion nucleic acid molecule of the disclosure comprises detecting a fragment of the fusion nucleic acid molecule comprising a breakpoint or fusion junction, e.g., one or more of the corresponding breakpoints described above and/or in the Examples herein.

In some embodiments of any of the methods provided herein, detecting an ABL1 fusion polypeptide of the disclosure comprises detecting a portion of the fusion polypeptide that is encoded by a fragment of an ABL1 fusion nucleic acid molecule that comprises a breakpoint or a fusion junction, e.g., one or more of the corresponding breakpoints described above and/or in the Examples herein.

In some embodiments, the methods further comprise providing an assessment of the ABL1 fusion nucleic acid molecule or ABL1 fusion polypeptide of the disclosure.

In some embodiments of any of the methods provided herein, the anti-cancer therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer comprising an ABL1 fusion nucleic acid molecule or an ABL1 fusion polypeptide, a treatment for cancer being tested in a clinical trial, a targeted therapy, a treatment being tested in a clinical trial for cancer comprising an ABL1 fusion nucleic acid molecule or an ABL1 fusion polypeptide, or any combination thereof, e.g., a described in further detail below. In some embodiments, the anti-cancer therapy is a kinase inhibitor, such as a kinase inhibitor described herein or known in the art. In some embodiments, the kinase inhibitor is a multi-kinase inhibitor or an ABL1-specific inhibitor known in the art or described herein. In some embodiments, the kinase inhibitor is a tyrosine kinase inhibitor. In some embodiments, the kinase inhibitor inhibits the kinase activity of an ABL1 polypeptide. In some embodiments, the anti-cancer therapy is an ABL1-targeted therapy, such as any of imatinib, nilotinib, dasatinib, ponatinib, bosutinib, asciminib, olverembatinib, radotinib or vodobatinib, and any combination thereof. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the nucleic acid inhibits the expression of an ABL1 fusion nucleic acid molecule or an ABL1 fusion polypeptide of the disclosure. In some embodiments, the nucleic acid comprises a double-stranded RNA (dsRNA), a small interfering RNA (siRNA), or a small hairpin RNA (shRNA), e.g., as described herein.

In some embodiments of any of the methods provided herein, the methods further comprise acquiring knowledge of or detecting in a sample from the individual a base substitution, a short insertion/deletion (indel), a copy number alteration, or a genomic rearrangement in one or more genes. In some embodiments, the one or more genes comprise the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKNIA, CDKNIB, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKARIA, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCA1P, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene, or any combination thereof. In some embodiments, the one or more genes comprise the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene, or any combination thereof.

In some embodiments of any of the methods provided herein, the treatment or the one or more treatment options further comprise an additional anti-cancer therapy. In some embodiments of any of the methods provided herein, the treatment or the one or more treatment options further comprise administering an additional anti-cancer therapy to the individual. In some embodiments, the additional anti-cancer therapy is any anti-cancer therapy known in the art or described herein. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

In some embodiments, the individual has been previously treated, or is being treated, for cancer with a treatment for cancer, e.g., an anti-cancer therapy described herein or any other anti-cancer therapy or treatment known in the art. In some embodiments, an ABL1 fusion nucleic acid molecule, and/or an ABL1 fusion polypeptide of the disclosure confers resistance of the cancer to the prior or current treatment for cancer.

In some embodiments of any of the methods provided herein, the cancer is a carcinoma, a sarcoma, a lymphoma, a leukemia, a myeloma, a germ cell cancer, or a blastoma. In some embodiments, the cancer is a solid tumor. In some embodiments, the cancer is a hematologic malignancy. In some embodiments, the cancer is a B cell cancer, multiple myeloma, bone marrow multiple myeloma, melanoma, breast cancer, lung cancer, bronchus cancer, colorectal cancer, prostate cancer, pancreatic cancer, stomach cancer, ovarian cancer, ovary epithelial carcinoma, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, testis germ cell tumor, biliary tract cancer, small bowel cancer, appendix cancer, salivary gland cancer, thyroid gland cancer, adrenal gland cancer, osteosarcoma, soft tissue osteosarcoma, chondrosarcoma, a cancer of hematological tissue, an adenocarcinoma, an inflammatory myofibroblastic tumor, a gastrointestinal stromal tumor (GIST), colon cancer, myelodysplastic syndrome (MDS), myeloproliferative disorder (MPD), acute lymphocytic leukemia (ALL) or bone marrow leukemia lymphocytic acute (ALL), bone marrow leukemia T cell acute (T-ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinoma, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, squamous cell cancer, non-squamous cell cancer, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, lung adenocarcinoma, non-small cell lung cancer, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, peritoneum adenocarcinoma, soft tissue malignant peripheral nerve sheath tumor (MPNST) or a carcinoid tumor.

In some embodiments, the cancer is acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some embodiments, the methods of the disclosure further comprise detecting the presence or absence of a cancer in a sample from the individual. In some embodiments, the methods further comprise administering an effective amount of anti-cancer therapy to the individual, e.g., an anti-cancer therapy described herein, e.g., an ABL1-targeted therapy.

In some embodiments, any of the cancers described herein comprise any of the ABL1 fusion nucleic acid molecules of the disclosure, e.g., an ABL1 fusion nucleic acid molecule described above and/or in the Examples herein. In other embodiments, any of the cancers described herein comprise any of the ABL1 fusion polypeptides of the disclosure, e.g., an ABL1 fusion polypeptide described above and/or in the Examples herein.

In some embodiments, the methods provided herein comprise acquiring knowledge of or detecting any of the ABL1 fusion nucleic acid molecules of the disclosure, e.g., an ABL1 fusion nucleic acid molecule described above and/or in the Examples herein, in a sample from an individual having any cancer known in the art, or any of the cancers described herein. In some embodiments, the methods provided herein comprise acquiring knowledge of or detecting any of the ABL1 fusion polypeptides of the disclosure, e.g., an ABL1 fusion polypeptide described above and/or in the Examples herein, in a sample from an individual having any cancer known in the art, or any of the cancers described herein.

In some embodiments, a cancer provided in Table 2 in Example 1 herein comprises the corresponding ABL1 fusion nucleic acid molecule and/or ABL1 fusion polypeptide as described in Table 2.

In some embodiments, an ABL1 fusion nucleic acid molecule of the disclosure is an EHMT1-ABL1 fusion nucleic acid molecule, wherein the EHMT1-ABL1 fusion nucleic acid molecule: comprises, in 5′ to 3′ direction, at least a portion of an EHMT1 gene fused to at least a portion of an ABL1 gene; comprises or results from a breakpoint within intron 4 of an EHMT1 gene and/or a breakpoint within intron 1 of an ABL1 gene; comprises a fusion between exon 4, or a portion thereof, of an EHMT1 gene and exon 2, or a portion thereof, of an ABL1 gene; comprises or results from a 5′ breakpoint within chromosomal coordinates chr9: 140630475-140630606 and/or a 3′ breakpoint within chromosomal coordinates chr9: 133729408-133729648; comprises, in 5′ to 3′ direction, exons 1-3 and exon 4, or a portion thereof, of an EHMT1 gene, fused to exon 2, or a portion thereof, and exons 3-11 of an ABL1 gene; encodes an EHMT1-ABL1 fusion polypeptide comprising the amino acid sequence of SEQ ID NO: 23, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 23; comprises the nucleotide sequence of SEQ ID NO: 2, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 2; and/or encodes the ABL1 kinase domain, or a portion of the ABL1 kinase domain having kinase activity; wherein the cancer is a lung cancer, such as a lung adenocarcinoma.

In some embodiments, an ABL1 fusion nucleic acid molecule of the disclosure is a MED27-ABL1 fusion nucleic acid molecule, wherein the MED27-ABL1 fusion nucleic acid molecule: comprises, in 5′ to 3′ direction, at least a portion of a MED27 gene fused to at least a portion of an ABL1 gene; comprises or results from a breakpoint within intron 4 of a MED27 gene and/or a breakpoint within intron 4 of an ABL1 gene; comprises a fusion between exon 4, or a portion thereof, of a MED27 gene and exon 5, or a portion thereof, of an ABL1 gene; comprises or results from a 5′ breakpoint within chromosomal coordinates chr9: 134774423-134776006 and/or a 3′ breakpoint within chromosomal coordinates chr9: 133747364-133747762; comprises, in 5′ to 3′ direction, exons 1-3 and exon 4, or a portion thereof, of a MED27 gene, fused to exon 5, or a portion thereof, and exons 6-11 of an ABL1 gene; encodes a MED27-ABL1 fusion polypeptide comprising the amino acid sequence of SEQ ID NO: 24, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 24; comprises the nucleotide sequence of SEQ ID NO: 3, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 3; and/or encodes the ABL1 kinase domain, or a portion of the ABL1 kinase domain having kinase activity; wherein the cancer is an ovarian cancer, such as an ovary epithelial carcinoma (e.g., not otherwise specified).

In some embodiments, an ABL1 fusion nucleic acid molecule of the disclosure is an RFFL-ABL1 fusion nucleic acid molecule, wherein the RFFL-ABL1 fusion nucleic acid molecule: comprises, in 5′ to 3′ direction, at least a portion of an RFFL gene fused to at least a portion of an ABL1 gene; comprises a fusion between exon 5, or a portion thereof, of an RFFL gene and exon 2, or a portion thereof, of an ABL1 gene; comprises or results from a 5′ breakpoint within chromosomal coordinates chr17: 33343379-33343419 and/or a 3′ breakpoint within chromosomal coordinates chr9: 133729485-133729525; comprises, in 5′ to 3′ direction, exons 1-4 and exon 5, or a portion thereof, of an RFFL gene, fused to exon 2, or a portion thereof, and exons 3-11 of an ABL1 gene; encodes an RFFL-ABL1 fusion polypeptide comprising the amino acid sequence of SEQ ID NO: 25, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 25; comprises the nucleotide sequence of SEQ ID NO: 4, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 4; and/or encodes the ABL1 kinase domain, or a portion of the ABL1 kinase domain having kinase activity; wherein the cancer is a nerve sheath tumor, such as a soft tissue malignant peripheral nerve sheath tumor (MPNST).

In some embodiments, an ABL1 fusion nucleic acid molecule of the disclosure is an SLC27A4-ABL1 fusion nucleic acid molecule, wherein the SLC27A4-ABL1 fusion nucleic acid molecule: comprises, in 5′ to 3′ direction, at least a portion of an SLC27A4 gene fused to at least a portion of an ABL1 gene; comprises or results from a breakpoint within intron 12 of an SLC27A4 gene and/or a breakpoint within intron 5 of an ABL1 gene; comprises a fusion between exon 12, or a portion thereof, of an SLC27A4 gene and exon 6, or a portion thereof, of an ABL1 gene; comprises or results from a 5′ breakpoint within chromosomal coordinates chr9: 131119603-131119801 and/or a 3′ breakpoint within chromosomal coordinates chr9: 133748211-133748501; comprises, in 5′ to 3′ direction, exons 1-11 and exon 12, or a portion thereof, of an SLC27A4 gene, fused to exon 6, or a portion thereof, and exons 7-11 of an ABL1 gene; encodes an SLC27A4-ABL1 fusion polypeptide comprising the amino acid sequence of SEQ ID NO: 26, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 26; comprises the nucleotide sequence of SEQ ID NO: 5, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 5; and/or encodes the ABL1 kinase domain, or a portion of the ABL1 kinase domain having kinase activity; wherein the cancer is a testicular cancer, such as a testis germ cell tumor, e.g., a mixed testis germ cell tumor.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 13 of a BCR gene and/or a breakpoint within intron 1 of an ABL1 gene; comprises a fusion between exon 13, or a portion thereof, of a BCR gene and exon 2, or a portion thereof, of an ABL1 gene; comprises or results from a 5′ breakpoint within chromosomal coordinates chr22: 23632033-23632321 and/or a 3′ breakpoint within chromosomal coordinates chr9: 133591666-133591902; comprises, in 5′ to 3′ direction, exons 1-12 and exon 13, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 27, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 27; comprises the nucleotide sequence of SEQ ID NO: 6, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 6; and/or encodes the ABL1 kinase domain, or a portion of the ABL1 kinase domain having kinase activity; wherein the cancer is a lung cancer, such as a lung adenocarcinoma.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23634537-23634672 and/or a 3′ breakpoint within chromosomal coordinates chr9: 133672815-133673059; comprises, in 5′ to 3′ direction, exons 1-13 and exon 14, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 28, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 28; comprises the nucleotide sequence of SEQ ID NO: 7, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 7; and/or encodes the ABL1 kinase domain, or a portion of the ABL1 kinase domain having kinase activity; wherein the cancer is a lung cancer, such as a lung adenocarcinoma.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23634041-23634323 and/or a 3′ breakpoint within chromosomal coordinates chr9: 133708182-133708567; comprises, in 5′ to 3′ direction, exons 1-13 and exon 14, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 29, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 29; comprises the nucleotide sequence of SEQ ID NO: 8, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 8; and/or encodes the ABL1 kinase domain, or a portion of the ABL1 kinase domain having kinase activity; wherein the cancer is a lung cancer, such as a lung adenocarcinoma.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23632683-23632948 and/or a 3′ breakpoint within chromosomal coordinates chr9: 133613427-133613626; comprises, in 5′ to 3′ direction, exons 1-13 and exon 14, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 30, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 30; comprises the nucleotide sequence of SEQ ID NO: 9, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 9; and/or encodes the ABL1 kinase domain, or a portion of the ABL1 kinase domain having kinase activity; wherein the cancer is a lung cancer, such as a lung adenocarcinoma.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23634011-23634460 and/or a 3′ breakpoint within chromosomal coordinates chr9: 133645693-133646017; comprises, in 5′ to 3′ direction, exons 1-13 and exon 14, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 32, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 32; comprises the nucleotide sequence of SEQ ID NO: 11, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 11; and/or encodes the ABL1 kinase domain, or a portion of the ABL1 kinase domain having kinase activity; wherein the cancer is a head and neck cancer, such as a head and neck squamous cell carcinoma.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23634211-23634632 and/or a 3′ breakpoint within chromosomal coordinates chr9: 133619101-133619395; comprises, in 5′ to 3′ direction, exons 1-13 and exon 14, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 33, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 33; comprises the nucleotide sequence of SEQ ID NO: 12, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 12; and/or encodes the ABL1 kinase domain, or a portion of the ABL1 kinase domain having kinase activity; wherein the cancer is a head and neck cancer, such as a head and neck squamous cell carcinoma.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 13 of a BCR gene and/or a breakpoint within intron 1 of an ABL1 gene; comprises a fusion between exon 13, or a portion thereof, of a BCR gene and exon 2, or a portion thereof, of an ABL1 gene; comprises or results from a 5′ breakpoint within chromosomal coordinates chr22: 23631722-23631762 and/or a 3′ breakpoint within chromosomal coordinates chr9: 133729484-133729524; comprises, in 5′ to 3′ direction, exons 1-12 and exon 13, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 34, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 34; comprises the nucleotide sequence of SEQ ID NO: 13, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 13; and/or encodes the ABL1 kinase domain, or a portion of the ABL1 kinase domain having kinase activity; wherein the cancer is a bone cancer, such as a bone sarcoma (e.g., not otherwise specified).

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23633346-23634163 and/or a 3′ breakpoint within chromosomal coordinates chr9: 133609201-133609909; comprises, in 5′ to 3′ direction, exons 1-13 and exon 14, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 35, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 35; comprises the nucleotide sequence of SEQ ID NO: 14, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 14; and/or encodes the ABL1 kinase domain, or a portion of the ABL1 kinase domain having kinase activity; wherein the cancer is an esophageal cancer, such as an esophagus adenocarcinoma.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23633432-23633796 and/or a 3′ breakpoint within chromosomal coordinates chr9: 133623903-133624461; comprises, in 5′ to 3′ direction, exons 1-13 and exon 14, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 36, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 36; comprises the nucleotide sequence of SEQ ID NO: 15, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 15; and/or encodes the ABL1 kinase domain, or a portion of the ABL1 kinase domain having kinase activity; wherein the cancer is a gastrointestinal tract cancer, such as a gastroesophageal junction adenocarcinoma.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23633837-23634208 and/or a 3′ breakpoint within chromosomal coordinates chr9: 133665426-133665727; comprises, in 5′ to 3′ direction, exons 1-13 and exon 14, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 37, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 37; comprises the nucleotide sequence of SEQ ID NO: 16, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 16; and/or encodes the ABL1 kinase domain, or a portion of the ABL1 kinase domain having kinase activity; wherein the cancer is a liver cancer, such as a liver cholangiocarcinoma.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23632858-23633068 and/or a 3′ breakpoint within chromosomal coordinates chr9: 133701595-133701846; comprises, in 5′ to 3′ direction, exons 1-13 and exon 14, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 38, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 38; comprises the nucleotide sequence of SEQ ID NO: 17, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 17; and/or encodes the ABL1 kinase domain, or a portion of the ABL1 kinase domain having kinase activity; wherein the cancer is a pancreatic cancer, such as a pancreas ductal adenocarcinoma.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23633208-23633695 and/or a 3′ breakpoint within chromosomal coordinates chr9: 133702617-133702992; comprises, in 5′ to 3′ direction, exons 1-13 and exon 14, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 39, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 39; comprises the nucleotide sequence of SEQ ID NO: 18, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 18; and/or encodes the ABL1 kinase domain, or a portion of the ABL1 kinase domain having kinase activity; wherein the cancer is an ovarian cancer, such as an ovary serous carcinoma.

In some embodiments, an ABL1 fusion nucleic acid molecule of the disclosure is a NUP214-ABL1 fusion nucleic acid molecule, wherein the NUP214-ABL1 fusion nucleic acid molecule: comprises, in 5′ to 3′ direction, at least a portion of a NUP214 gene fused to at least a portion of an ABL1 gene; comprises a fusion between exon 34, or a portion thereof, of a NUP214 gene and exon 3, or a portion thereof, of an ABL1 gene; comprises or results from a 5′ breakpoint within chromosomal coordinates chr9: 134106039-134106079 and/or a 3′ breakpoint within chromosomal coordinates chr9: 133730222-133730262; comprises, in 5′ to 3′ direction, exons 1-33 and exon 34, or a portion thereof, of a NUP214 gene, fused to exon 3, or a portion thereof, and exons 4-11 of an ABL1 gene; encodes a NUP214-ABL1 fusion polypeptide comprising the amino acid sequence of SEQ ID NO: 40, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 40; comprises the nucleotide sequence of SEQ ID NO: 19, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 19; and/or encodes the ABL1 kinase domain, or a portion of the ABL1 kinase domain having kinase activity; wherein the cancer is a leukemia, such as a bone marrow leukemia lymphocytic chronic (CLL).

In some embodiments, an ABL1 fusion nucleic acid molecule of the disclosure is an ABL1-AIF1L fusion nucleic acid molecule, wherein the ABL1-AIF1L fusion nucleic acid molecule: comprises, in 5′ to 3′ direction, at least a portion of an ABL1 gene fused to at least a portion of an AIF1L gene; comprises a fusion between exon 10, or a portion thereof, of an ABL1 gene and exon 4, or a portion thereof, of an AIF1L gene; comprises or results from a 5′ breakpoint within chromosomal coordinates chr9: 133755952-133755992 and/or a 3′ breakpoint within chromosomal coordinates chr9: 133989965-133990005; comprises, in 5′ to 3′ direction, exons 1-9 and exon 10, or a portion thereof, of an ABL1 gene, fused to exon 4, or a portion thereof, and exons 5-6 of an AIF1L gene; encodes an ABL1-AIF1L fusion polypeptide comprising the amino acid sequence of SEQ ID NO: 41, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 41; comprises the nucleotide sequence of SEQ ID NO: 20, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 20; and/or encodes the ABL1 kinase domain, or a portion of the ABL1 kinase domain having kinase activity; wherein the cancer is a sarcoma, such as a soft tissue sarcoma (e.g., not otherwise specified).

In some embodiments, an ABL1 fusion nucleic acid molecule of the disclosure is an RCSD1-ABL1 fusion nucleic acid molecule, wherein the RCSD1-ABL1 fusion nucleic acid molecule: comprises, in 5′ to 3′ direction, at least a portion of an RCSD1 gene fused to at least a portion of an ABL1 gene; comprises a fusion between exon 4, or a portion thereof, of an RCSD1 gene and exon 4, or a portion thereof, of an ABL1 gene; comprises or results from a 5′ breakpoint within chromosomal coordinates chr1: 167654668-167654708 and/or a 3′ breakpoint within chromosomal coordinates chr9: 133738173-133738213; comprises, in 5′ to 3′ direction, exons 1-3 and exon 4, or a portion thereof, of an RCSD1 gene, fused to exon 4, or a portion thereof, and exons 5-11 of an ABL1 gene; encodes an RCSD1-ABL1 fusion polypeptide comprising the amino acid sequence of SEQ ID NO: 42, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 42; comprises the nucleotide sequence of SEQ ID NO: 21, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 21; and/or encodes the ABL1 kinase domain, or a portion of the ABL1 kinase domain having kinase activity; wherein the cancer is a leukemia, such as a bone marrow leukemia lymphocytic chronic (CLL).

In some embodiments, an ABL1 fusion nucleic acid molecule of the disclosure is an APOOL-ABL1 fusion nucleic acid molecule, wherein the APOOL-ABL1 fusion nucleic acid molecule: comprises, in 5′ to 3′ direction, at least a portion of an APOOL gene fused to at least a portion of an ABL1 gene; comprises or results from a breakpoint within intron 2 of an APOOL gene and/or a breakpoint within exon 2 of an ABL1 gene; comprises a fusion between exon 2, or a portion thereof, of an APOOL gene and exon 2, or a portion thereof, of an ABL1 gene; comprises or results from a 5′ breakpoint within chromosomal coordinates chrX: 84302705-84302904 and/or a 3′ breakpoint within chromosomal coordinates chr9: 133729584-133729766; comprises, in 5′ to 3′ direction, exon 1 and exon 2, or a portion thereof, of an APOOL gene, fused to exon 2, or a portion thereof, and exons 3-11 of an ABL1 gene; and/or encodes the ABL1 kinase domain, or a portion of the ABL1 kinase domain having kinase activity; wherein the cancer is a lung cancer, such as a lung adenocarcinoma.

In some embodiments, an ABL1 fusion nucleic acid molecule of the disclosure is an ABL1-DNAH2 fusion nucleic acid molecule, wherein the ABL1-DNAH2 fusion nucleic acid molecule: comprises, in 5′ to 3′ direction, at least a portion of an ABL1 gene fused to at least a portion of a DNAH2 gene; comprises a fusion between exon 10, or a portion thereof, of an ABL1 gene and exon 4, or a portion thereof, of a DNAH2 gene; comprises or results from a 5′ breakpoint within chromosomal coordinates chr9: 133755953-133755993 and/or a 3′ breakpoint within chromosomal coordinates chr17: 7636399-7636439; comprises, in 5′ to 3′ direction, exons 1-9 and exon 10, or a portion thereof, of an ABL1 gene, fused to exon 4, or a portion thereof, and exons 5-85 of a DNAH2 gene; and/or encodes the ABL1 kinase domain, or a portion of the ABL1 kinase domain having kinase activity; wherein the cancer is a sarcoma, such as a soft tissue sarcoma (e.g., not otherwise specified).

In some embodiments, an ABL1 fusion nucleic acid molecule of the disclosure is a RHEX/C1orf186-ABL1 fusion nucleic acid molecule, wherein the RHEX/C1orf186-ABL1 fusion nucleic acid molecule: comprises, in 5′ to 3′ direction, at least a portion of a RHEX/Clorf186-gene fused to at least a portion of an ABL1 gene; comprises a fusion between exon 1, or a portion thereof, of a RHEX/Clorf186 gene and exon 2, or a portion thereof, of an ABL1 gene; comprises or results from a 5′ breakpoint within chromosomal coordinates chr1: 206288099-206288139 and/or a 3′ breakpoint within chromosomal coordinates chr9: 133729488-133729528; comprises, in 5′ to 3′ direction, exon 1, or a portion thereof, of a RHEX/C1orf186 gene, fused to exon 2, or a portion thereof, and exons 3-11 of an ABL1 gene; and/or encodes the ABL1 kinase domain, or a portion of the ABL1 kinase domain having kinase activity; wherein the cancer is a multiple myeloma (e.g., a bone marrow multiple myeloma).

In some embodiments, an ABL1 fusion nucleic acid molecule of the disclosure is a RALGPS1-ABL1 fusion nucleic acid molecule, wherein the RALGPS1-ABL1 fusion nucleic acid molecule: comprises, in 5′ to 3′ direction, at least a portion of a RALGPS1 gene fused to at least a portion of an ABL1 gene; comprises or results from a breakpoint within intron 1 of a RALGPS1 gene and/or a breakpoint within intron 2 of an ABL1 gene; comprises a fusion between exon 1, or a portion thereof, of a RALGPS1 gene and exon 3, or a portion thereof, of an ABL1 gene; comprises or results from a 5′ breakpoint within chromosomal coordinates chr9: 129694023-129694282 and/or a 3′ breakpoint within chromosomal coordinates chr9: 133730118-133730529; comprises, in 5′ to 3′ direction, exon 1, or a portion thereof, of a RALGPS1 gene, fused to exon 3, or a portion thereof, and exons 4-11 of an ABL1 gene; and/or encodes the ABL1 kinase domain, or a portion of the ABL1 kinase domain having kinase activity; wherein the cancer is a melanoma (e.g., an unknown primary melanoma).

In some embodiments, an ABL1 fusion nucleic acid molecule of the disclosure is a TSC1-ABL1 fusion nucleic acid molecule, wherein the TSC1-ABL1 fusion nucleic acid molecule: comprises, in 5′ to 3′ direction, at least a portion of a TSC1 gene fused to at least a portion of an ABL1 gene; comprises a fusion between exon 19, or a portion thereof, of a TSC1 gene and exon 2, or a portion thereof, of an ABL1 gene; comprises or results from a 5′ breakpoint within chromosomal coordinates chr9:135,776,998-135,777,038 and/or a 3′ breakpoint within chromosomal coordinates chr9: 133729491-133729531; comprises, in 5′ to 3′ direction, exons 1-18 and exon 19, or a portion thereof, of a TSC1 gene, fused to exon 2, or a portion thereof, and exons 3-11 of an ABL1 gene; and/or encodes the ABL1 kinase domain, or a portion of the ABL1 kinase domain having kinase activity; wherein the cancer is a nerve sheath tumor, such as a soft tissue malignant peripheral nerve sheath tumor (MPNST).

In some embodiments, an ABL1 fusion nucleic acid molecule of the disclosure is a TSPAN18-ABL1 fusion nucleic acid molecule, wherein the TSPAN18-ABL1 fusion nucleic acid molecule: comprises, in 5′ to 3′ direction, at least a portion of a TSPAN18 gene fused to at least a portion of an ABL1 gene; comprises a fusion between exon 2, or a portion thereof, of a TSPAN18 gene and exon 2, or a portion thereof, of an ABL1 gene; comprises or results from a 5′ breakpoint within chromosomal coordinates chr11: 44881929-44881969 and/or a 3′ breakpoint within chromosomal coordinates chr9: 133729517-133729557; comprises, in 5′ to 3′ direction, exon 1 and exon 2, or a portion thereof, of a TSPAN18 gene, fused to exon 2, or a portion thereof, and exons 3-11 of an ABL1 gene; and/or encodes the ABL1 kinase domain, or a portion of the ABL1 kinase domain having kinase activity; wherein the cancer is an osteosarcoma, such as a soft tissue osteosarcoma (extraskeletal).

In some embodiments, an ABL1 fusion nucleic acid molecule of the disclosure is a ZNF804B-ABL1 fusion nucleic acid molecule, wherein the ZNF804B-ABL1 fusion nucleic acid molecule: comprises, in 5′ to 3′ direction, at least a portion of a ZNF804B gene fused to at least a portion of an ABL1 gene; comprises or results from a breakpoint within intron 1 of a ZNF804B gene and/or a breakpoint within intron 1 of an ABL1 gene; comprises a fusion between exon 1, or a portion thereof, of a ZNF804B gene and exon 2, or a portion thereof, of an ABL1 gene; comprises or results from a 5′ breakpoint within chromosomal coordinates chr7: 88521265-88521397 and/or a 3′ breakpoint within chromosomal coordinates chr9: 133729414-133729519; comprises, in 5′ to 3′ direction, exon 1, or a portion thereof, of a ZNF804B gene, fused to exon 2, or a portion thereof, and exons 3-11 of an ABL1 gene; and/or encodes the ABL1 kinase domain, or a portion of the ABL1 kinase domain having kinase activity; wherein the cancer is a skin cancer, such as a skin melanoma.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 13 of a BCR gene and/or a breakpoint within intron 1 of an ABL1 gene; comprises a fusion between exon 13, or a portion thereof, of a BCR gene and exon 2, or a portion thereof, of an ABL1 gene; comprises or results from a 5′ breakpoint within chromosomal coordinates chr22: 23631687-23632492 and/or a 3′ breakpoint within chromosomal coordinates chr9: 133604425-133604577; comprises, in 5′ to 3′ direction, exons 1-12 and exon 13, or a portion thereof, of a BCR gene, fused to exon 2, or a portion thereof, and exons 3-11 of an ABL1 gene; and/or encodes the ABL1 kinase domain, or a portion of the ABL1 kinase domain having kinase activity; wherein the cancer is a head and neck cancer, such as a head and neck squamous cell carcinoma (HNSCC).

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23632288-23632656 and/or a 3′ breakpoint within chromosomal coordinates chr9: 133663291-133663682; comprises, in 5′ to 3′ direction, exons 1-13 and exon 14, or a portion thereof, of a BCR gene, fused to exon 2, or a portion thereof, and exons 3-11 of an ABL1 gene; and/or encodes the ABL1 kinase domain, or a portion of the ABL1 kinase domain having kinase activity; wherein the cancer is a head and neck cancer, such as a head and neck squamous cell carcinoma (HNSCC).

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23634171-23634647 and/or a 3′ breakpoint within chromosomal coordinates chr9: 133638886-133639512; comprises, in 5′ to 3′ direction, exons 1-13 and exon 14, or a portion thereof, of a BCR gene, fused to exon 2, or a portion thereof, and exons 3-11 of an ABL1 gene; and/or encodes the ABL1 kinase domain, or a portion of the ABL1 kinase domain having kinase activity; wherein the cancer is a lung cancer, such as a lung squamous cell carcinoma.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 14 of a BCR gene and/or a breakpoint within intron 1 of an ABL1 gene; comprises a fusion between exon 14, or a portion thereof, of a BCR gene and exon 2, or a portion thereof, of an ABL1 gene; comprises or results from a 5′ breakpoint within chromosomal coordinates chr22: 23633910-23634678 and/or a 3′ breakpoint within chromosomal coordinates chr9: 133724778-133725351; comprises, in 5′ to 3′ direction, exons 1-13 and exon 14, or a portion thereof, of a BCR gene, fused to exon 2, or a portion thereof, and exons 3-11 of an ABL1 gene; and/or encodes the ABL1 kinase domain, or a portion of the ABL1 kinase domain having kinase activity; wherein the cancer is a lung cancer, such as a lung squamous cell carcinoma.

In some embodiments, an ABL1 fusion nucleic acid molecule of the disclosure is an ABL1-NUP214 fusion nucleic acid molecule, wherein the ABL1-NUP214 fusion nucleic acid molecule: comprises, in 5′ to 3′ direction, at least a portion of an ABL1 gene fused to at least a portion of a NUP214 gene; comprises a fusion between exon 1, or a portion thereof, of an ABL1 gene and exon 31, or a portion thereof, of a NUP214 gene; comprises or results from a 5′ breakpoint within chromosomal coordinates chr9: 134090583-134090623 and/or a 3′ breakpoint within chromosomal coordinates chr9: 133710830-133710870; and/or comprises, in 5′ to 3′ direction, exon 1, or a portion thereof, of an ABL1 gene, fused to exon 31, or a portion thereof, and exons 32-36 of a NUP214 gene; wherein the cancer is a liposarcoma, such as a soft tissue liposarcoma.

In some embodiments, an ABL1 fusion nucleic acid molecule of the disclosure is an AIF1L-ABL1 fusion nucleic acid molecule, wherein the AIF1L-ABL1 fusion nucleic acid molecule: comprises, in 5′ to 3′ direction, at least a portion of an AIF1L gene fused to at least a portion of an ABL1 gene; comprises or results from a breakpoint within intron 2 of an AIF1L gene and/or a breakpoint within intron 1 of an ABL1 gene; comprises a fusion between exon 2, or a portion thereof, of an AIF1L gene and exon 2, or a portion thereof, of an ABL1 gene; comprises or results from a 5′ breakpoint within chromosomal coordinates chr9: 133978032-133978137 and/or a 3′ breakpoint within chromosomal coordinates chr9: 133729409-133729618; comprises, in 5′ to 3′ direction, exon 1 and exon 2, or a portion thereof, of an AIF1L gene, fused to exon 2, or a portion thereof, and exons 3-11 of an ABL1 gene; and/or encodes the ABL1 kinase domain, or a portion of the ABL1 kinase domain having kinase activity; wherein the cancer is an ovarian cancer, such as an ovary serous carcinoma.

In some embodiments, an ABL1 fusion nucleic acid molecule of the disclosure is an EXOSC2-ABL1 fusion nucleic acid molecule, wherein the EXOSC2-ABL1 fusion nucleic acid molecule: comprises, in 5′ to 3′ direction, at least a portion of an EXOSC2 gene fused to at least a portion of an ABL1 gene; comprises or results from a breakpoint within exon 9 of an EXOSC2 gene and/or a breakpoint within exon 1 of an ABL1 gene; comprises a fusion between exon 9, or a portion thereof, of an EXOSC2 gene and exon 1, or a portion thereof, of an ABL1 gene; comprises or results from a 5′ breakpoint within chromosomal coordinates chr9: 133579154-133579531 and/or a 3′ breakpoint within chromosomal coordinates chr9: 133589786-133589985; comprises, in 5′ to 3′ direction, exons 1-8 and exon 9, or a portion thereof, of an EXOSC2 gene, fused to exon 1, or a portion thereof, and exons 2-11 of an ABL1 gene; and/or encodes the ABL1 kinase domain, or a portion of the ABL1 kinase domain having kinase activity; wherein the cancer is a peritoneum cancer, such as a peritoneum adenocarcinoma.

In some embodiments, an ABL1 fusion nucleic acid molecule of the disclosure is a NUP214-ABL1 fusion nucleic acid molecule, wherein the NUP214-ABL1 fusion nucleic acid molecule: comprises, in 5′ to 3′ direction, at least a portion of a NUP214 gene fused to at least a portion of an ABL1 gene; comprises or results from a breakpoint within intron 34 of a NUP214 gene and/or a breakpoint within exon 3 of an ABL1 gene; comprises a fusion between exon 34, or a portion thereof, of a NUP214 gene and exon 3, or a portion thereof, of an ABL1 gene; comprises or results a 5′ breakpoint within chromosomal coordinates chr9: 134106080-134106120 and/or a 3′ breakpoint within chromosomal coordinates chr9: 133730195-133730235; comprises, in 5′ to 3′ direction, exons 1-33 and exon 34, or a portion thereof, of a NUP214 gene, fused to exon 3, or a portion thereof, and exons 4-11 of an ABL1 gene; and/or encodes the ABL1 kinase domain, or a portion of the ABL1 kinase domain having kinase activity; wherein the cancer is a leukemia, such as a bone marrow leukemia B cell acute (B-ALL).

In some embodiments, an ABL1 fusion nucleic acid molecule of the disclosure is a NUP214-ABL1 fusion nucleic acid molecule, wherein the NUP214-ABL1 fusion nucleic acid molecule: comprises, in 5′ to 3′ direction, at least a portion of a NUP214 gene fused to at least a portion of an ABL1 gene; comprises a fusion between exon 34, or a portion thereof, of a NUP214 gene and exon 3, or a portion thereof, of an ABL1 gene; comprises or results from a 5′ breakpoint within chromosomal coordinates chr9: 134106030-134106070 and/or a 3′ breakpoint within chromosomal coordinates chr9: 133730218-133730258; comprises, in 5′ to 3′ direction, exons 1-33 and exon 34, or a portion thereof, of a NUP214 gene, fused to exon 3, or a portion thereof, and exons 4-11 of an ABL1 gene; and/or encodes the ABL1 kinase domain, or a portion of the ABL1 kinase domain having kinase activity; wherein the cancer is a leukemia, such as a bone marrow leukemia B cell acute (B-ALL).

In some embodiments, an ABL1 fusion nucleic acid molecule of the disclosure is a NUP214-ABL1 fusion nucleic acid molecule, wherein the NUP214-ABL1 fusion nucleic acid molecule: comprises, in 5′ to 3′ direction, at least a portion of a NUP214 gene fused to at least a portion of an ABL1 gene; comprises a fusion between exon 34, or a portion thereof, of a NUP214 gene and exon 3, or a portion thereof, of an ABL1 gene; comprises or results from a 5′ breakpoint within chromosomal coordinates chr9: 134106046-134106086 and/or a 3′ breakpoint within chromosomal coordinates chr9: 133730203-133730243; comprises, in 5′ to 3′ direction, exons 1-33 and exon 34, or a portion thereof, of a NUP214 gene, fused to exon 3, or a portion thereof, and exons 4-11 of an ABL1 gene; and/or encodes the ABL1 kinase domain, or a portion of the ABL1 kinase domain having kinase activity; wherein the cancer is a leukemia, such as a bone marrow leukemia B cell acute (B-ALL).

In some embodiments, an ABL1 fusion nucleic acid molecule of the disclosure is a NUP214-ABL1 fusion nucleic acid molecule, wherein the NUP214-ABL1 fusion nucleic acid molecule: comprises, in 5′ to 3′ direction, at least a portion of a NUP214 gene fused to at least a portion of an ABL1 gene; comprises a fusion between exon 34, or a portion thereof, of a NUP214 gene and exon 3, or a portion thereof, of an ABL1 gene; comprises or results from a 5′ breakpoint within chromosomal coordinates chr9: 134106028-134106068 and/or a 3′ breakpoint within chromosomal coordinates chr9: 133730234-133730274; comprises, in 5′ to 3′ direction, exons 1-33 and exon 34, or a portion thereof, of a NUP214 gene, fused to exon 3, or a portion thereof, and exons 4-11 of an ABL1 gene; and/or encodes the ABL1 kinase domain, or a portion of the ABL1 kinase domain having kinase activity; wherein the cancer is a leukemia, such as a bone marrow leukemia lymphocytic acute (ALL).

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises a fusion between exon 1, or a portion thereof, of a BCR gene and exon 3, or a portion thereof, of an ABL1 gene; comprises or results from a 5′ breakpoint within chromosomal coordinates chr22: 23524272-23524312 and/or a 3′ breakpoint within chromosomal coordinates chr9: 133730218-133730258; comprises, in 5′ to 3′ direction, exon 1, or a portion thereof, of a BCR gene, fused to exon 3, or a portion thereof, and exons 4-11 of an ABL1 gene; and/or encodes the ABL1 kinase domain, or a portion of the ABL1 kinase domain having kinase activity; wherein the cancer is a leukemia, such as a bone marrow leukemia lymphocytic acute (ALL).

In some embodiments, an ABL1 fusion nucleic acid molecule of the disclosure is an SNX2-ABL1 fusion nucleic acid molecule, wherein the SNX2-ABL1 fusion nucleic acid molecule: comprises, in 5′ to 3′ direction, at least a portion of an SNX2 gene fused to at least a portion of an ABL1 gene; comprises a fusion between exon 3, or a portion thereof, of an SNX2 gene and exon 4, or a portion thereof, of an ABL1 gene; comprises or results from a 5′ breakpoint within chromosomal coordinates chr5: 122135451-122135491 and/or a 3′ breakpoint within chromosomal coordinates chr9: 133738222-133738262; comprises, in 5′ to 3′ direction, exons 1-2 and exon 3, or a portion thereof, of an SNX2 gene, fused to exon 4, or a portion thereof, and exons 5-11 of an ABL1 gene; and/or encodes the ABL1 kinase domain, or a portion of the ABL1 kinase domain having kinase activity; wherein the cancer is a leukemia, such as a bone marrow leukemia B cell acute (B-ALL).

In some embodiments, an ABL1 fusion nucleic acid molecule of the disclosure is an SNX2-ABL1 fusion nucleic acid molecule, wherein the SNX2-ABL1 fusion nucleic acid molecule: comprises, in 5′ to 3′ direction, at least a portion of an SNX2 gene fused to at least a portion of an ABL1 gene; comprises a fusion between exon 3, or a portion thereof, of an SNX2 gene and exon 4, or a portion thereof, of an ABL1 gene; comprises or results from a 5′ breakpoint within chromosomal coordinates chr5: 122135445-122135485 and/or a 3′ breakpoint within chromosomal coordinates chr9: 133738214-133738254; comprises, in 5′ to 3′ direction, exons 1-2 and exon 3, or a portion thereof, of an SNX2 gene, fused to exon 4, or a portion thereof, and exons 5-11 of an ABL1 gene; and/or encodes the ABL1 kinase domain, or a portion of the ABL1 kinase domain having kinase activity; wherein the cancer is a leukemia-lymphoma, such as a B-lymphoblastic leukemia-lymphoma.

In some embodiments, an ABL1 fusion nucleic acid molecule of the disclosure is a BCR-ABL1 fusion nucleic acid molecule, wherein the BCR-ABL1 fusion nucleic acid molecule: comprises, in 5′ to 3′ direction, at least a portion of a BCR gene fused to at least a portion of an ABL1 gene; comprises or results from a breakpoint within intron 12 of a BCR gene and/or a breakpoint within intron 1 of an ABL1 gene; comprises a fusion between exon 12, or a portion thereof, of a BCR gene and exon 2, or a portion thereof, of an ABL1 gene; comprises or results from a 5′ breakpoint within chromosomal coordinates chr22: 23631693-23632188 and/or a 3′ breakpoint within chromosomal coordinates chr9: 133607683-133608245; comprises, in 5′ to 3′ direction, exons 1-11 and exon 12, or a portion thereof, of a BCR gene, fused to exon 2, or a portion thereof, and exons 3-11 of an ABL1 gene; encodes a BCR-ABL1 fusion polypeptide comprising the amino acid sequence of SEQ ID NO: 31, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 31; comprises the nucleotide sequence of SEQ ID NO: 10, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 10; and/or encodes the ABL1 kinase domain, or a portion of the ABL1 kinase domain having kinase activity; wherein the cancer is a lung cancer, such as a lung adenocarcinoma.

In some embodiments, an ABL1 fusion nucleic acid molecule of the disclosure is a BMP2K-ABL1 fusion nucleic acid molecule, wherein the BMP2K-ABL1 fusion nucleic acid molecule: comprises in 5′ to 3′ direction, at least a portion of a BMP2K gene fused to at least a portion of an ABL1 gene; comprises a fusion between exon 15, or a portion thereof, of a BMP2K gene and exon 3, or a portion thereof, of an ABL1 gene; comprises or results from a 5′ breakpoint within chromosomal coordinates chr4: 79800022-79800062 and/or a 3′ breakpoint within chromosomal coordinates chr9: 133730205-133730245; comprises, in 5′ to 3′ direction, exons 1-14 and exon 15, or a portion thereof, of a BMP2K gene, fused to exon 3, or a portion thereof, and exons 4-11 of an ABL1 gene; and/or encodes the ABL1 kinase domain, or a portion of the ABL1 kinase domain having kinase activity; wherein the cancer is a leukemia, such as a bone marrow leukemia T cell acute (T-ALL).

In some embodiments, an ABL1 fusion polypeptide of the disclosure is an EHMT1-ABL1 fusion polypeptide, wherein the EHMT1-ABL1 fusion polypeptide: is encoded by an ABL1 fusion nucleic acid molecule comprising, in 5′ to 3′ direction, at least a portion of an EHMT1 gene fused to at least a portion of an ABL1 gene; is encoded by an ABL1 fusion nucleic acid molecule comprising or resulting from a breakpoint within intron 4 of an EHMT1 gene and/or a breakpoint within intron 1 of an ABL1 gene; comprises a fusion between an amino acid sequence encoded by exon 4, or a portion thereof, of an EHMT1 gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr9: 140630475-140630606 and/or a 3′ breakpoint within chromosomal coordinates chr9: 133729408-133729648; comprises, in the N- to C-terminus direction, an amino acid sequence encoded by exons 1-3 and exon 4, or a portion thereof, of an EHMT1 gene, fused to an amino acid sequence encoded by exon 2, or a portion thereof, and exons 3-11 of an ABL1 gene; is encoded by an ABL1 fusion nucleic acid molecule comprising, in the 5′ to 3′ direction, exons 1-3 and exon 4, or a portion thereof, of an EHMT1 gene, fused to exon 2, or a portion thereof, and exons 3-11 of an ABL1 gene; comprises the amino acid sequence of SEQ ID NO: 23, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 23; is encoded by the nucleotide sequence of SEQ ID NO: 2, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 2; and/or comprises the ABL1 kinase domain, or a portion of the ABL1 kinase domain having kinase activity; wherein the cancer is a lung cancer, such as a lung adenocarcinoma.

In some embodiments, an ABL1 fusion polypeptide of the disclosure is a MED27-ABL1 fusion polypeptide, wherein the MED27-ABL1 fusion polypeptide: is encoded by an ABL1 fusion nucleic acid molecule comprising, in 5′ to 3′ direction, at least a portion of a MED27 gene fused to at least a portion of an ABL1 gene; is encoded by an ABL1 fusion nucleic acid molecule comprising or resulting from a breakpoint within intron 4 of a MED27 gene and/or a breakpoint within intron 4 of an ABL1 gene; comprises a fusion between an amino acid sequence encoded by exon 4, or a portion thereof, of a MED27 gene and an amino acid sequence encoded by exon 5, or a portion thereof, of an ABL1 gene; is encoded by an ABL1 fusion nucleic acid molecule comprising or resulting from a 5′ breakpoint within chromosomal coordinates chr9: 134774423-134776006 and/or a 3′ breakpoint within chromosomal coordinates chr9: 133747364-133747762; comprises, in the N- to C-terminus direction, an amino acid sequence encoded by exons 1-3 and exon 4, or a portion thereof, of a MED27 gene, fused to an amino acid sequence encoded by exon 5, or a portion thereof, and exons 6-11 of an ABL1 gene; is encoded by an ABL1 fusion nucleic acid molecule comprising, in the 5′ to 3′ direction, exons 1-3 and exon 4, or a portion thereof, of a MED27 gene, fused to exon 5, or a portion thereof, and exons 6-11 of an ABL1 gene; comprises the amino acid sequence of SEQ ID NO: 24, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 24; is encoded by the nucleotide sequence of SEQ ID NO: 3, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 3; and/or comprises the ABL1 kinase domain, or a portion of the ABL1 kinase domain having kinase activity; wherein the cancer is an ovarian cancer, such as an ovary epithelial carcinoma (e.g., not otherwise specified).

In some embodiments, an ABL1 fusion polypeptide of the disclosure is an RFFL-ABL1 fusion polypeptide, wherein the RFFL-ABL1 fusion polypeptide: is encoded by an ABL1 fusion nucleic acid molecule comprising, in 5′ to 3′ direction, at least a portion of an RFFL gene fused to at least a portion of an ABL1 gene; comprises a fusion between an amino acid sequence encoded by exon 5, or a portion thereof, of an RFFL gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr17: 33343379-33343419 and/or a 3′ breakpoint within chromosomal coordinates chr9: 133729485-133729525; comprises, in the N- to C-terminus direction, an amino acid sequence encoded by exons 1-4 and exon 5, or a portion thereof, of an RFFL gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-4 and exon 5, or a portion thereof, of an RFFL gene, fused to exon 2, or a portion thereof, and exons 3-11 of an ABL1 gene; comprises the amino acid sequence of SEQ ID NO: 25, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 25; is encoded by the nucleotide sequence of SEQ ID NO: 4, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 4; and/or comprises the ABL1 kinase domain, or a portion of the ABL1 kinase domain having kinase activity; wherein the cancer is a nerve sheath tumor, such as a soft tissue malignant peripheral nerve sheath tumor (MPNST).

In some embodiments, an ABL1 fusion polypeptide of the disclosure is an SLC27A4-ABL1 fusion polypeptide, wherein the SLC27A4-ABL1 fusion polypeptide: is encoded by an ABL1 fusion nucleic acid molecule comprising, in 5′ to 3′ direction, at least a portion of an SLC27A4 gene fused to at least a portion of an ABL1 gene; is encoded by an ABL1 fusion nucleic acid molecule comprising or resulting from a breakpoint within intron 12 of an SLC27A4 gene and/or a breakpoint within intron 5 of an ABL1 gene; comprises a fusion between an amino acid sequence encoded by exon 12, or a portion thereof, of an SLC27A4 gene and an amino acid sequence encoded by exon 6, or a portion thereof, of an ABL1 gene; is encoded by an ABL1 fusion nucleic acid molecule comprising or resulting from a 5′ breakpoint within chromosomal coordinates chr9: 131119603-131119801 and/or a 3′ breakpoint within chromosomal coordinates chr9: 133748211-133748501; comprises, in the N- to C-terminus direction, an amino acid sequence encoded by exons 1-11 and exon 12, or a portion thereof, of an SLC27A4 gene, fused to an amino acid sequence encoded by exon 6, or a portion thereof, and exons 7-11 of an ABL1 gene; is encoded by an ABL1 fusion nucleic acid molecule comprising, in 5′ to 3′ direction, exons 1-11 and exon 12, or a portion thereof, of an SLC27A4 gene, fused to exon 6, or a portion thereof, and exons 7-11 of an ABL1 gene; comprises the amino acid sequence of SEQ ID NO: 26, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 26; is encoded by the nucleotide sequence of SEQ ID NO: 5, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 5; and/or comprises the ABL1 kinase domain, or a portion of the ABL1 kinase domain having kinase activity; wherein the cancer is a testicular cancer, such as a testis germ cell tumor, e.g., a mixed testis germ cell tumor.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 13 of a BCR gene and/or a breakpoint within intron 1 of an ABL1 gene; comprises a fusion between an amino acid sequence encoded by exon 13, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23632033-23632321 and/or a 3′ breakpoint within chromosomal coordinates chr9: 133591666-133591902; comprises, in the N- to C-terminus direction, an amino acid sequence encoded by exons 1-12 and exon 13, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-12 and exon 13, or a portion thereof, of a BCR gene, fused to exon 2, or a portion thereof, and exons 3-11 of an ABL1 gene; comprises the amino acid sequence of SEQ ID NO: 27, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 27; is encoded by the nucleotide sequence of SEQ ID NO: 6, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 6; and/or comprises the ABL1 kinase domain, or a portion of the ABL1 kinase domain having kinase activity; wherein the cancer is a lung cancer, such as a lung adenocarcinoma.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23634537-23634672 and/or a 3′ breakpoint within chromosomal coordinates chr9: 133672815-133673059; comprises, in then N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; comprises the amino acid sequence of SEQ ID NO: 28, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 28; is encoded by the nucleotide sequence of SEQ ID NO: 7, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 7; and/or comprises the ABL1 kinase domain, or a portion of the ABL1 kinase domain having kinase activity; wherein the cancer is a lung cancer, such as a lung adenocarcinoma.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23634041-23634323 and/or a 3′ breakpoint within chromosomal coordinates chr9: 133708182-133708567; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; comprises the amino acid sequence of SEQ ID NO: 29, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 29; is encoded by the nucleotide sequence of SEQ ID NO: 8, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 8; and/or comprises the ABL1 kinase domain, or a portion of the ABL1 kinase domain having kinase activity; wherein the cancer is a lung cancer, such as a lung adenocarcinoma.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23632683-23632948 and/or a 3′ breakpoint within chromosomal coordinates chr9: 133613427-133613626; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; comprises the amino acid sequence of SEQ ID NO: 30, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 30; is encoded by the nucleotide sequence of SEQ ID NO: 9, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 9; and/or comprises the ABL1 kinase domain, or a portion of the ABL1 kinase domain having kinase activity; wherein the cancer is a lung cancer, such as a lung adenocarcinoma.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23634011-23634460 and/or a 3′ breakpoint within chromosomal coordinates chr9: 133645693-133646017; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; comprises the amino acid sequence of SEQ ID NO: 32, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 32; is encoded by the nucleotide sequence of SEQ ID NO: 11, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 11; and/or comprises the ABL1 kinase domain, or a portion of the ABL1 kinase domain having kinase activity; wherein the cancer is a head and neck cancer, such as a head and neck squamous cell carcinoma.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23634211-23634632 and/or a 3′ breakpoint within chromosomal coordinates chr9: 133619101-133619395; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; comprises the amino acid sequence of SEQ ID NO: 33, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 33; is encoded by the nucleotide sequence of SEQ ID NO: 12, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 12; and/or comprises the ABL1 kinase domain, or a portion of the ABL1 kinase domain having kinase activity; wherein the cancer is a head and neck cancer, such as a head and neck squamous cell carcinoma.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 13 of a BCR gene and/or a breakpoint within intron 1 of an ABL1 gene; comprises a fusion between an amino acid sequence encoded by exon 13, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23631722-23631762 and/or a 3′ breakpoint within chromosomal coordinates chr9: 133729484-133729524; comprises, in the N- to C-terminus direction, an amino acid sequence encoded by exons 1-12 and exon 13, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-12 and exon 13, or a portion thereof, of a BCR gene, fused to exon 2, or a portion thereof, and exons 3-11 of an ABL1 gene; comprises the amino acid sequence of SEQ ID NO: 34, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 34; is encoded by the nucleotide sequence of SEQ ID NO: 13, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 13; and/or comprises the ABL1 kinase domain, or a portion of the ABL1 kinase domain having kinase activity; wherein the cancer is a bone cancer, such as a bone sarcoma (e.g., not otherwise specified).

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23633346-23634163 and/or a 3′ breakpoint within chromosomal coordinates chr9: 133609201-133609909; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; comprises the amino acid sequence of SEQ ID NO: 35, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 35; is encoded by the nucleotide sequence of SEQ ID NO: 14, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 14; and/or comprises the ABL1 kinase domain, or a portion of the ABL1 kinase domain having kinase activity; wherein the cancer is an esophageal cancer, such as an esophagus adenocarcinoma.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23633432-23633796 and/or a 3′ breakpoint within chromosomal coordinates chr9: 133623903-133624461; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; comprises the amino acid sequence of SEQ ID NO: 36, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 36; is encoded by the nucleotide sequence of SEQ ID NO: 15, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 15; and/or comprises the ABL1 kinase domain, or a portion of the ABL1 kinase domain having kinase activity; wherein the cancer is a gastrointestinal tract cancer, such as a gastroesophageal junction adenocarcinoma.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23633837-23634208 and/or a 3′ breakpoint within chromosomal coordinates chr9: 133665426-133665727; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; comprises the amino acid sequence of SEQ ID NO: 37, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 37; is encoded by the nucleotide sequence of SEQ ID NO: 16, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 16; and/or comprises the ABL1 kinase domain, or a portion of the ABL1 kinase domain having kinase activity; wherein the cancer is a liver cancer, such as a liver cholangiocarcinoma.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23632858-23633068 and/or a 3′ breakpoint within chromosomal coordinates chr9: 133701595-133701846; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; comprises the amino acid sequence of SEQ ID NO: 38, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 38; is encoded by the nucleotide sequence of SEQ ID NO: 17, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 17; and/or comprises the ABL1 kinase domain, or a portion of the ABL1 kinase domain having kinase activity; wherein the cancer is a pancreatic cancer, such as a pancreas ductal adenocarcinoma.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23633208-23633695 and/or a 3′ breakpoint within chromosomal coordinates chr9: 133702617-133702992; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; comprises the amino acid sequence of SEQ ID NO: 39, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 39; is encoded by the nucleotide sequence of SEQ ID NO: 18, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 18; and/or comprises the ABL1 kinase domain, or a portion of the ABL1 kinase domain having kinase activity; wherein the cancer is an ovarian cancer, such as an ovary serous carcinoma.

In some embodiments, an ABL1 fusion polypeptide of the disclosure is a NUP214-ABL1 fusion polypeptide, wherein the NUP214-ABL1 fusion polypeptide: is encoded by an ABL1 fusion nucleic acid molecule comprising, in 5′ to 3′ direction, at least a portion of a NUP214 gene fused to at least a portion of an ABL1 gene; comprises a fusion between an amino acid sequence encoded by exon 34, or a portion thereof, of a NUP214 gene and an amino acid sequence encoded by exon 3, or a portion thereof, of an ABL1 gene; is encoded by an ABL1 fusion nucleic acid molecule comprising or resulting from a 5′ breakpoint within chromosomal coordinates chr9: 134106039-134106079 and/or a 3′ breakpoint within chromosomal coordinates chr9: 133730222-133730262; comprises, in the N- to C-terminus direction, an amino acid sequence encoded by exons 1-33 and exon 34, or a portion thereof, of a NUP214 gene, fused to an amino acid sequence encoded by exon 3, or a portion thereof, and exons 4-11 of an ABL1 gene; is encoded by an ABL1 fusion nucleic acid molecule comprising, in 5′ to 3′ direction, exons 1-33 and exon 34, or a portion thereof, of a NUP214 gene, fused to exon 3, or a portion thereof, and exons 4-11 of an ABL1 gene; comprises the amino acid sequence of SEQ ID NO: 40, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 40; is encoded by the nucleotide sequence of SEQ ID NO: 19, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 19; and/or comprises the ABL1 kinase domain, or a portion of the ABL1 kinase domain having kinase activity; wherein the cancer is a leukemia, such as a bone marrow leukemia lymphocytic chronic (CLL).

In some embodiments, an ABL1 fusion polypeptide of the disclosure is an ABL1-AIF1L fusion polypeptide, wherein the ABL1-AIF1L fusion polypeptide: is encoded by an ABL1 fusion nucleic acid molecule comprising, in 5′ to 3′ direction, at least a portion of an ABL1 gene fused to at least a portion of an AIF1L gene; comprises a fusion between an amino acid sequence encoded by exon 10, or a portion thereof, of an ABL1 gene and an amino acid sequence encoded by exon 4, or a portion thereof, of an AIF1L gene; is encoded by an ABL1 fusion nucleic acid molecule comprising or resulting from a 5′ breakpoint within chromosomal coordinates chr9: 133755952-133755992 and/or a 3′ breakpoint within chromosomal coordinates chr9: 133989965-133990005; comprises, in the N- to C-terminus direction, an amino acid sequence encoded by exons 1-9 and exon 10, or a portion thereof, of an ABL1 gene, fused to an amino acid sequence encoded by exon 4, or a portion thereof, and exons 5-6 of an AIF1L gene; is encoded by an ABL1 fusion nucleic acid molecule comprising, in 5′ to 3′ direction, exons 1-9 and exon 10, or a portion thereof, of an ABL1 gene, fused to exon 4, or a portion thereof, and exons 5-6 of an AIF1L gene; comprises the amino acid sequence of SEQ ID NO: 41, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 41; is encoded by the nucleotide sequence of SEQ ID NO: 20, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 20; and/or encodes the ABL1 kinase domain, or a portion of the ABL1 kinase domain having kinase activity; wherein the cancer is a sarcoma, such as a soft tissue sarcoma (e.g., not otherwise specified).

In some embodiments, an ABL1 fusion polypeptide of the disclosure is an RCSD1-ABL1 fusion polypeptide, wherein the RCSD1-ABL1 fusion polypeptide: is encoded by an ABL1 fusion nucleic acid molecule comprising, in 5′ to 3′ direction, at least a portion of an RCSD1 gene fused to at least a portion of an ABL1 gene; comprises a fusion between an amino acid sequence encoded by exon 4, or a portion thereof, of an RCSD1 gene and an amino acid sequence encoded by exon 4, or a portion thereof, of an ABL1 gene; is encoded by an ABL1 fusion nucleic acid molecule comprising or resulting from a 5′ breakpoint within chromosomal coordinates chr1: 167654668-167654708 and/or a 3′ breakpoint within chromosomal coordinates chr9: 133738173-133738213; comprises, in the N- to C-terminus direction, an amino acid sequence encoded by exons 1-3 and exon 4, or a portion thereof, of an RCSD1 gene, fused to an amino acid sequence encoded by exon 4, or a portion thereof, and exons 5-11 of an ABL1 gene; is encoded by an ABL1 fusion nucleic acid molecule comprising, in the 5′ to 3′ direction, exons 1-3 and exon 4, or a portion thereof, of an RCSD1 gene, fused to exon 4, or a portion thereof, and exons 5-11 of an ABL1 gene; comprises the amino acid sequence of SEQ ID NO: 42, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 42; is encoded by the nucleotide sequence of SEQ ID NO: 21, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 21; and/or comprises the ABL1 kinase domain, or a portion of the ABL1 kinase domain having kinase activity; wherein the cancer is a leukemia, such as a bone marrow leukemia lymphocytic chronic (CLL).

In some embodiments, an ABL1 fusion polypeptide of the disclosure is an APOOL-ABL1 fusion polypeptide, wherein the APOOL-ABL1 fusion polypeptide: is encoded by an ABL1 fusion nucleic acid molecule comprising, in 5′ to 3′ direction, at least a portion of an APOOL gene fused to at least a portion of an ABL1 gene; is encoded by an ABL1 fusion nucleic acid molecule comprising or resulting from a breakpoint within intron 2 of an APOOL gene and/or a breakpoint within exon 2 of an ABL1 gene; comprises a fusion between an amino acid sequence encoded by exon 2, or a portion thereof, of an APOOL gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chrX: 84302705-84302904 and/or a 3′ breakpoint within chromosomal coordinates chr9: 133729584-133729766; comprises, in the N- to C-terminus direction, an amino acid sequence encoded by exon 1 and exon 2, or a portion thereof, of an APOOL gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exon 1 and exon 2, or a portion thereof, of an APOOL gene, fused to exon 2, or a portion thereof, and exons 3-11 of an ABL1 gene; and/or comprises the ABL1 kinase domain, or a portion of the ABL1 kinase domain having kinase activity; wherein the cancer is a lung cancer, such as a lung adenocarcinoma.

In some embodiments, an ABL1 fusion polypeptide of the disclosure is an ABL1-DNAH2 fusion polypeptide, wherein the ABL1-DNAH2 fusion polypeptide: is encoded by an ABL1 fusion nucleic acid molecule comprising, in 5′ to 3′ direction, at least a portion of an ABL1 gene fused to at least a portion of a DNAH2 gene; comprises a fusion between an amino acid sequence encoded by exon 10, or a portion thereof, of an ABL1 gene and an amino acid sequence encoded by exon 4, or a portion thereof, of a DNAH2 gene; is encoded by an ABL1 fusion nucleic acid molecule comprising or resulting from a 5′ breakpoint within chromosomal coordinates chr9: 133755953-133755993 and/or a 3′ breakpoint within chromosomal coordinates chr17: 7636399-7636439; comprises, in the N- to C-terminus direction, an amino acid sequence encoded by exons 1-9 and exon 10, or a portion thereof, of an ABL1 gene, fused to an amino acid sequence encoded by exon 4, or a portion thereof, and exons 5-85 of a DNAH2 gene; is encoded by an ABL1 fusion nucleic acid molecule comprising, in 5′ to 3′ direction, exons 1-9 and exon 10, or a portion thereof, of an ABL1 gene, fused to exon 4, or a portion thereof, and exons 5-85 of a DNAH2 gene; and/or comprises the ABL1 kinase domain, or a portion of the ABL1 kinase domain having kinase activity; wherein the cancer is a sarcoma, such as a soft tissue sarcoma (e.g., not otherwise specified).

In some embodiments, an ABL1 fusion polypeptide of the disclosure is a RHEX/C1orf186-ABL1 fusion polypeptide, wherein the RHEX/C1orf186-ABL1 fusion polypeptide: is encoded by an ABL1 fusion nucleic acid molecule comprising, in 5′ to 3′ direction, at least a portion of a RHEX/C1orf186-gene fused to at least a portion of an ABL1 gene; comprises a fusion between an amino acid sequence encoded by exon 1, or a portion thereof, of a RHEX/Clorf186 gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr1: 206288099-206288139 and/or a 3′ breakpoint within chromosomal coordinates chr9: 133729488-133729528; comprises, in the N- to C-terminus direction, an amino acid sequence encoded by exon 1, or a portion thereof, of a RHEX/Clorf186 gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exon 1, or a portion thereof, of a RHEX/C1orf186 gene, fused to exon 2, or a portion thereof, and exons 3-11 of an ABL1 gene; and/or comprises the ABL1 kinase domain, or a portion of the ABL1 kinase domain having kinase activity; wherein the cancer is a multiple myeloma (e.g., a bone marrow multiple myeloma).

In some embodiments, an ABL1 fusion polypeptide of the disclosure is a RALGPS1-ABL1 fusion polypeptide, wherein the RALGPS1-ABL1 fusion polypeptide: is encoded by an ABL1 fusion nucleic acid molecule comprising, in 5′ to 3′ direction, at least a portion of a RALGPS1 gene fused to at least a portion of an ABL1 gene; is encoded by an ABL1 fusion nucleic acid molecule comprising or resulting from a breakpoint within intron 1 of a RALGPS1 gene and/or a breakpoint within intron 2 of an ABL1 gene; comprises a fusion between an amino acid sequence encoded by exon 1, or a portion thereof, of a RALGPS1 gene and an amino acid sequence encoded by exon 3, or a portion thereof, of an ABL1 gene; is encoded by an ABL1 fusion nucleic acid molecule comprising or resulting from a 5′ breakpoint within chromosomal coordinates chr9: 129694023-129694282 and/or a 3′ breakpoint within chromosomal coordinates chr9: 133730118-133730529; comprises, in the N- to C-terminus direction, an amino acid sequence encoded by exon 1, or a portion thereof, of a RALGPS1 gene, fused to an amino acid sequence encoded by exon 3, or a portion thereof, and exons 4-11 of an ABL1 gene; is encoded by an ABL1 fusion nucleic acid molecule comprising, in 5′ to 3′ direction, exon 1, or a portion thereof, of a RALGPS1 gene, fused to exon 3, or a portion thereof, and exons 4-11 of an ABL1 gene; and/or comprises the ABL1 kinase domain, or a portion of the ABL1 kinase domain having kinase activity; wherein the cancer is a melanoma (e.g., an unknown primary melanoma).

In some embodiments, an ABL1 fusion polypeptide of the disclosure is a TSC1-ABL1 fusion polypeptide, wherein the TSC1-ABL1 fusion polypeptide: is encoded by an ABL1 fusion nucleic acid molecule comprising, in 5′ to 3′ direction, at least a portion of a TSC1 gene fused to at least a portion of an ABL1 gene; comprises a fusion between an amino acid sequence encoded by exon 19, or a portion thereof, of a TSC1 gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr9:135,776,998-135,777,038 and/or a 3′ breakpoint within chromosomal coordinates chr9: 133729491-133729531; comprises, in the N- to C-terminus direction, an amino acid sequence encoded by exons 1-18 and exon 19, or a portion thereof, of a TSC1 gene, fused to an amino acid sequence encoded by exon 2, or a portion thereof, and exons 3-11 of an ABL1 gene; is encoded by an ABL1 fusion nucleic acid molecule comprising, in the 5′ to 3′ direction, exons 1-18 and exon 19, or a portion thereof, of a TSC1 gene, fused to exon 2, or a portion thereof, and exons 3-11 of an ABL1 gene; and/or comprises the ABL1 kinase domain, or a portion of the ABL1 kinase domain having kinase activity; wherein the cancer is a nerve sheath tumor, such as a soft tissue malignant peripheral nerve sheath tumor (MPNST).

In some embodiments, an ABL1 fusion polypeptide of the disclosure is a TSPAN18-ABL1 fusion polypeptide, wherein the TSPAN18-ABL1 fusion polypeptide: is encoded by an ABL1 fusion nucleic acid molecule comprising, in 5′ to 3′ direction, at least a portion of a TSPAN18 gene fused to at least a portion of an ABL1 gene; comprises a fusion between an amino acid sequence encoded by exon 2, or a portion thereof, of a TSPAN18 gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr11: 44881929-44881969 and/or a 3′ breakpoint within chromosomal coordinates chr9: 133729517-133729557; comprises, in the N- to C-terminus direction, an amino acid sequence encoded by exon 1 and exon 2, or a portion thereof, of a TSPAN18 gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exon 1 and exon 2, or a portion thereof, of a TSPAN18 gene, fused to exon 2, or a portion thereof, and exons 3-11 of an ABL1 gene; and/or comprises the ABL1 kinase domain, or a portion of the ABL1 kinase domain having kinase activity; wherein the cancer is an osteosarcoma, such as a soft tissue osteosarcoma (extraskeletal).

In some embodiments, an ABL1 fusion polypeptide of the disclosure is a ZNF804B-ABL1 fusion polypeptide, wherein the ZNF804B-ABL1 fusion polypeptide: is encoded by an ABL1 fusion nucleic acid molecule comprising, in 5′ to 3′ direction, at least a portion of a ZNF804B gene fused to at least a portion of an ABL1 gene; is encoded by an ABL1 fusion nucleic acid molecule comprising or resulting from a breakpoint within intron 1 of a ZNF804B gene and/or a breakpoint within intron 1 of an ABL1 gene; comprises a fusion between an amino acid sequence encoded by exon 1, or a portion thereof, of a ZNF804B gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr7: 88521265-88521397 and/or a 3′ breakpoint within chromosomal coordinates chr9: 133729414-133729519; comprises, in the N- to C-terminus direction, an amino acid sequence encoded by exon 1, or a portion thereof, of a ZNF804B gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exon 1, or a portion thereof, of a ZNF804B gene, fused to exon 2, or a portion thereof, and exons 3-11 of an ABL1 gene; and/or comprises the ABL1 kinase domain, or a portion of the ABL1 kinase domain having kinase activity; wherein the cancer is a skin cancer, such as a skin melanoma.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 13 of a BCR gene and/or a breakpoint within intron 1 of an ABL1 gene; comprises a fusion between an amino acid sequence encoded by exon 13, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23631687-23632492 and/or a 3′ breakpoint within chromosomal coordinates chr9: 133604425-133604577; comprises, in the N- to C-terminus direction, an amino acid sequence encoded by exons 1-12 and exon 13, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-12 and exon 13, or a portion thereof, of a BCR gene, fused to exon 2, or a portion thereof, and exons 3-11 of an ABL1 gene; and/or comprises the ABL1 kinase domain, or a portion of the ABL1 kinase domain having kinase activity; wherein the cancer is a head and neck cancer, such as a head and neck squamous cell carcinoma (HNSCC).

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23632288-23632656 and/or a 3′ breakpoint within chromosomal coordinates chr9: 133663291-133663682; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; and/or comprises the ABL1 kinase domain, or a portion of the ABL1 kinase domain having kinase activity; wherein the cancer is a head and neck cancer, such as a head and neck squamous cell carcinoma (HNSCC).

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23634171-23634647 and/or a 3′ breakpoint within chromosomal coordinates chr9: 133638886-133639512; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; and/or comprises the ABL1 kinase domain, or a portion of the ABL1 kinase domain having kinase activity; wherein the cancer is a lung cancer, such as a lung squamous cell carcinoma.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 14 of a BCR gene and/or a breakpoint within intron 1 of an ABL1 gene; comprises a fusion between an amino acid sequence encoded by exon 14, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23633910-23634678 and/or a 3′ breakpoint within chromosomal coordinates chr9: 133724778-133725351; comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-13 and exon 14, or a portion thereof, of a BCR gene, fused to exon 2, or a portion thereof, and exons 3-11 of an ABL1 gene; and/or comprises the ABL1 kinase domain, or a portion of the ABL1 kinase domain having kinase activity; wherein the cancer is a lung cancer, such as a lung squamous cell carcinoma.

In some embodiments, an ABL1 fusion polypeptide of the disclosure is an ABL1-NUP214 fusion polypeptide, wherein the ABL1-NUP214 fusion polypeptide: is encoded by an ABL1 fusion nucleic acid molecule comprising, in 5′ to 3′ direction, at least a portion of an ABL1 gene fused to at least a portion of a NUP214 gene; comprises a fusion between an amino acid sequence encoded by exon 1, or a portion thereof, of an ABL1 gene and an amino acid sequence encoded by exon 31, or a portion thereof, of a NUP214 gene; is encoded by an ABL1 fusion nucleic acid molecule comprising or resulting from a 5′ breakpoint within chromosomal coordinates chr9: 134090583-134090623 and/or a 3′ breakpoint within chromosomal coordinates chr9: 133710830-133710870; comprises, in the N- to C-terminus direction, an amino acid sequence encoded by exon 1, or a portion thereof, of an ABL1 gene, fused to an amino acid sequence encoded by exon 31, or a portion thereof, and exons 32-36 of a NUP214 gene; and/or is encoded by an ABL1 fusion nucleic acid molecule comprising, in 5′ to 3′ direction, exon 1, or a portion thereof, of an ABL1 gene, fused to exon 31, or a portion thereof, and exons 32-36 of a NUP214 gene; wherein the cancer is a liposarcoma, such as a soft tissue liposarcoma.

In some embodiments, an ABL1 fusion polypeptide of the disclosure is an AIF1L-ABL1 fusion polypeptide, wherein the AIF1L-ABL1 fusion polypeptide: is encoded by an ABL1 fusion nucleic acid molecule comprising, in 5′ to 3′ direction, at least a portion of an AIF1L gene fused to at least a portion of an ABL1 gene; is encoded by an ABL1 fusion nucleic acid molecule comprising or resulting from a breakpoint within intron 2 of an AIF1L gene and/or a breakpoint within intron 1 of an ABL1 gene; comprises a fusion between an amino acid sequence encoded by exon 2, or a portion thereof, of an AIF1L gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr9: 133978032-133978137 and/or a 3′ breakpoint within chromosomal coordinates chr9: 133729409-133729618; comprises, in the N- to C-terminus direction, an amino acid sequence encoded by exon 1 and exon 2, or a portion thereof, of an AIF1L gene, fused to an amino acid sequence encoded by exon 2, or a portion thereof, and exons 3-11 of an ABL1 gene; is encoded by an ABL1-fusion nucleic acid molecule comprising, in 5′ to 3′ direction, exon 1 and exon 2, or a portion thereof, of an AIF1L gene, fused to exon 2, or a portion thereof, and exons 3-11 of an ABL1 gene; and/or comprises the ABL1 kinase domain, or a portion of the ABL1 kinase domain having kinase activity; wherein the cancer is an ovarian cancer, such as an ovary serous carcinoma.

In some embodiments, an ABL1 fusion polypeptide of the disclosure is an EXOSC2-ABL1 fusion polypeptide, wherein the EXOSC2-ABL1 fusion polypeptide: is encoded by an ABL1 fusion nucleic acid molecule comprising, in 5′ to 3′ direction, at least a portion of an EXOSC2 gene fused to at least a portion of an ABL1 gene; is encoded by an ABL1 fusion nucleic acid molecule comprising or resulting from a breakpoint within exon 9 of an EXOSC2 gene and/or a breakpoint within exon 1 of an ABL1 gene; comprises a fusion between an amino acid sequence encoded by exon 9, or a portion thereof, of an EXOSC2 gene and an amino acid sequence encoded by exon 1, or a portion thereof, of an ABL1 gene; is encoded by an ABL1 fusion nucleic acid molecule comprising or resulting from a 5′ breakpoint within chromosomal coordinates chr9: 133579154-133579531 and/or a 3′ breakpoint within chromosomal coordinates chr9: 133589786-133589985; comprises, in the N- to C-terminus direction, an amino acid sequence encoded by exons 1-8 and exon 9, or a portion thereof, of an EXOSC2 gene, fused to an amino acid sequence encoded by exon 1, or a portion thereof, and exons 2-11 of an ABL1 gene; is encoded by an ABL1 fusion nucleic acid molecule comprising, in the 5′ to 3′ direction, exons 1-8 and exon 9, or a portion thereof, of an EXOSC2 gene, fused to exon 1, or a portion thereof, and exons 2-11 of an ABL1 gene; and/or comprises the ABL1 kinase domain, or a portion of the ABL1 kinase domain having kinase activity; wherein the cancer is a peritoneum cancer, such as a peritoneum adenocarcinoma.

In some embodiments, an ABL1 fusion polypeptide of the disclosure is a NUP214-ABL1 fusion polypeptide, wherein the NUP214-ABL1 fusion polypeptide: is encoded by an ABL1 fusion nucleic acid molecule comprising, in 5′ to 3′ direction, at least a portion of a NUP214 gene fused to at least a portion of an ABL1 gene; is encoded by an ABL1 fusion nucleic acid molecule comprising or resulting from a breakpoint within intron 34 of a NUP214 gene and/or a breakpoint within exon 3 of an ABL1 gene; comprises a fusion between an amino acid sequence encoded by exon 34, or a portion thereof, of a NUP214 gene and an amino acid sequence encoded by exon 3, or a portion thereof, of an ABL1 gene; is encoded by an ABL1 fusion nucleic acid molecule comprising or resulting from a 5′ breakpoint within chromosomal coordinates chr9: 134106080-134106120 and/or a 3′ breakpoint within chromosomal coordinates chr9: 133730195-133730235; comprises, in the N- to C-terminus direction, an amino acid sequence encoded by exons 1-33 and exon 34, or a portion thereof, of a NUP214 gene, fused to an amino acid sequence encoded by exon 3, or a portion thereof, and exons 4-11 of an ABL1 gene; is encoded by an ABL1 fusion nucleic acid molecule comprising, in 5′ to 3′ direction, exons 1-33 and exon 34, or a portion thereof, of a NUP214 gene, fused to exon 3, or a portion thereof, and exons 4-11 of an ABL1 gene; and/or comprises the ABL1 kinase domain, or a portion of the ABL1 kinase domain having kinase activity; wherein the cancer is a leukemia, such as a bone marrow leukemia B cell acute (B-ALL).

In some embodiments, an ABL1 fusion polypeptide of the disclosure is a NUP214-ABL1 fusion polypeptide, wherein the NUP214-ABL1 fusion polypeptide: is encoded by an ABL1 fusion nucleic acid molecule comprising, in 5′ to 3′ direction, at least a portion of a NUP214 gene fused to at least a portion of an ABL1 gene; comprises a fusion between an amino acid sequence encoded by exon 34, or a portion thereof, of a NUP214 gene and an amino acid sequence encoded by exon 3, or a portion thereof, of an ABL1 gene; is encoded by an ABL1 fusion nucleic acid molecule comprising or resulting from a 5′ breakpoint within chromosomal coordinates chr9: 134106030-134106070 and/or a 3′ breakpoint within chromosomal coordinates chr9: 133730218-133730258; comprises, in the N- to C-terminus direction, an amino acid sequence encoded by exons 1-33 and exon 34, or a portion thereof, of a NUP214 gene, fused to an amino acid sequence encoded by exon 3, or a portion thereof, and exons 4-11 of an ABL1 gene; is encoded by an ABL1 fusion nucleic acid molecule comprising, in 5′ to 3′ direction, exons 1-33 and exon 34, or a portion thereof, of a NUP214 gene, fused to exon 3, or a portion thereof, and exons 4-11 of an ABL1 gene; and/or comprises the ABL1 kinase domain, or a portion of the ABL1 kinase domain having kinase activity; wherein the cancer is a leukemia, such as a bone marrow leukemia B cell acute (B-ALL).

In some embodiments, an ABL1 fusion polypeptide of the disclosure is a NUP214-ABL1 fusion polypeptide, wherein the NUP214-ABL1 fusion polypeptide: is encoded by an ABL1 fusion nucleic acid molecule comprising, in 5′ to 3′ direction, at least a portion of a NUP214 gene fused to at least a portion of an ABL1 gene; comprises a fusion between an amino acid sequence encoded by exon 34, or a portion thereof, of a NUP214 gene and an amino acid sequence encoded by exon 3, or a portion thereof, of an ABL1 gene; is encoded by an ABL1 fusion nucleic acid molecule comprising or resulting from a 5′ breakpoint within chromosomal coordinates chr9: 134106046-134106086 and/or a 3′ breakpoint within chromosomal coordinates chr9: 133730203-133730243; comprises, in the N- to C-terminus direction, an amino acid sequence encoded by exons 1-33 and exon 34, or a portion thereof, of a NUP214 gene, fused to an amino acid sequence encoded by exon 3, or a portion thereof, and exons 4-11 of an ABL1 gene; is encoded by an ABL1 fusion nucleic acid molecule comprising, in 5′ to 3′ direction, exons 1-33 and exon 34, or a portion thereof, of a NUP214 gene, fused to exon 3, or a portion thereof, and exons 4-11 of an ABL1 gene; and/or comprises the ABL1 kinase domain, or a portion of the ABL1 kinase domain having kinase activity; wherein the cancer is a leukemia, such as a bone marrow leukemia B cell acute (B-ALL).

In some embodiments, an ABL1 fusion polypeptide of the disclosure is a NUP214-ABL1 fusion polypeptide, wherein the NUP214-ABL1 fusion polypeptide: is encoded by an ABL1 fusion nucleic acid molecule comprising, in 5′ to 3′ direction, at least a portion of a NUP214 gene fused to at least a portion of an ABL1 gene; comprises a fusion between an amino acid sequence encoded by exon 34, or a portion thereof, of a NUP214 gene and an amino acid sequence encoded by exon 3, or a portion thereof, of an ABL1 gene; is encoded by an ABL1 fusion nucleic acid molecule comprising or resulting from a 5′ breakpoint within chromosomal coordinates chr9: 134106028-134106068 and/or a 3′ breakpoint within chromosomal coordinates chr9: 133730234-133730274; comprises, in the N- to C-terminus direction, an amino acid sequence encoded by exons 1-33 and exon 34, or a portion thereof, of a NUP214 gene, fused to an amino acid sequence encoded by exon 3, or a portion thereof, and exons 4-11 of an ABL1 gene; is encoded by an ABL1 fusion nucleic acid molecule comprising, in 5′ to 3′ direction, exons 1-33 and exon 34, or a portion thereof, of a NUP214 gene, fused to exon 3, or a portion thereof, and exons 4-11 of an ABL1 gene; and/or comprises the ABL1 kinase domain, or a portion of the ABL1 kinase domain having kinase activity; wherein the cancer is a leukemia, such as a bone marrow leukemia lymphocytic acute (ALL).

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; comprises a fusion between an amino acid sequence encoded by exon 1, or a portion thereof, of a BCR gene and an amino acid sequence encoded by exon 3, or a portion thereof, of an ABL1 gene; is encoded by an ABL1 fusion nucleic acid molecule comprising or resulting from a 5′ breakpoint within chromosomal coordinates chr22: 23524272-23524312 and/or a 3′ breakpoint within chromosomal coordinates chr9: 133730218-133730258; comprises, in the N- to C-terminus direction, an amino acid sequence encoded by exon 1, or a portion thereof, of a BCR gene, fused to an amino acid sequence encoded by exon 3, or a portion thereof, and exons 4-11 of an ABL1 gene; is encoded by an ABL1 fusion nucleic acid molecule comprising, in 5′ to 3′ direction, exon 1, or a portion thereof, of a BCR gene, fused to exon 3, or a portion thereof, and exons 4-11 of an ABL1 gene; and/or comprises the ABL1 kinase domain, or a portion of the ABL1 kinase domain having kinase activity; wherein the cancer is a leukemia, such as a bone marrow leukemia lymphocytic acute (ALL).

In some embodiments, an ABL1 fusion polypeptide of the disclosure is an SNX2-ABL1 fusion polypeptide, wherein the SNX2-ABL1 fusion polypeptide: is encoded by an ABL1 fusion nucleic acid molecule comprising, in 5′ to 3′ direction, at least a portion of an SNX2 gene fused to at least a portion of an ABL1 gene; comprises a fusion between an amino acid sequence encoded by exon 3, or a portion thereof, of an SNX2 gene and an amino acid sequence encoded by exon 4, or a portion thereof, of an ABL1 gene; is encoded by an ABL1 fusion nucleic acid molecule comprising or resulting from a 5′ breakpoint within chromosomal coordinates chr5: 122135451-122135491 and/or a 3′ breakpoint within chromosomal coordinates chr9: 133738222-133738262; comprises, in the N- to C-terminus direction, an amino acid sequence encoded by exons 1-2 and exon 3, or a portion thereof, of an SNX2 gene, fused to an amino acid sequence encoded by exon 4, or a portion thereof, and exons 5-11 of an ABL1 gene; is encoded by an ABL1 fusion nucleic acid molecule comprising, in 5′ to 3′ direction, exons 1-2 and exon 3, or a portion thereof, of an SNX2 gene, fused to exon 4, or a portion thereof, and exons 5-11 of an ABL1 gene; and/or comprises the ABL1 kinase domain, or a portion of the ABL1 kinase domain having kinase activity; wherein the cancer is a leukemia, such as a bone marrow leukemia B cell acute (B-ALL).

In some embodiments, an ABL1 fusion polypeptide of the disclosure is an SNX2-ABL1 fusion polypeptide, wherein the SNX2-ABL1 fusion polypeptide: is encoded by an ABL1 fusion nucleic acid molecule comprising, in 5′ to 3′ direction, at least a portion of an SNX2 gene fused to at least a portion of an ABL1 gene; comprises a fusion between an amino acid sequence encoded by exon 3, or a portion thereof, of an SNX2 gene and an amino acid sequence encoded by exon 4, or a portion thereof, of an ABL1 gene; is encoded by an ABL1 fusion nucleic acid molecule comprising or resulting from a 5′ breakpoint within chromosomal coordinates chr5: 122135445-122135485 and/or a 3′ breakpoint within chromosomal coordinates chr9: 133738214-133738254; comprises, in the N- to C-terminus direction, an amino acid sequence encoded by exons 1-2 and exon 3, or a portion thereof, of an SNX2 gene, fused to an amino acid sequence encoded by exon 4, or a portion thereof, and exons 5-11 of an ABL1 gene; is encoded by an ABL1 fusion nucleic acid molecule comprising, in 5′ to 3′ direction, exons 1-2 and exon 3, or a portion thereof, of an SNX2 gene, fused to exon 4, or a portion thereof, and exons 5-11 of an ABL1 gene; and/or comprises the ABL1 kinase domain, or a portion of the ABL1 kinase domain having kinase activity; wherein the cancer is a leukemia-lymphoma, such as a B-lymphoblastic leukemia-lymphoma.

In some embodiments, an ABL1 fusion polypeptide of the disclosure is a BCR-ABL1 fusion polypeptide, wherein the BCR-ABL1 fusion polypeptide: is encoded by an ABL1 fusion nucleic acid molecule comprising, in 5′ to 3′ direction, at least a portion of a BCR gene fused to at least a portion of an ABL1 gene; is encoded by an ABL1 fusion nucleic acid molecule comprising or resulting from a breakpoint within intron 12 of a BCR gene and/or a breakpoint within intron 1 of an ABL1 gene; comprises a fusion between an amino acid sequence encoded by exon 12, or a portion thereof, of a BCR gene and an amino acid sequence encoded by exon 2, or a portion thereof, of an ABL1 gene; is encoded by an ABL1 fusion nucleic acid molecule comprising or resulting from a 5′ breakpoint within chromosomal coordinates chr22: 23631693-23632188 and/or a 3′ breakpoint within chromosomal coordinates chr9: 133607683-133608245; comprises, in the N- to C-terminus direction, an amino acid sequence encoded by exons 1-11 and exon 12, or a portion thereof, of a BCR gene, fused to an amino acid sequence encoded by exon 2, or a portion thereof, and exons 3-11 of an ABL1 gene; is encoded by an ABL1 fusion nucleic acid molecule comprising, in 5′ to 3′ direction, exons 1-11 and exon 12, or a portion thereof, of a BCR gene, fused to exon 2, or a portion thereof, and exons 3-11 of an ABL1 gene; comprises the amino acid sequence of SEQ ID NO: 31, or an amino acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence of SEQ ID NO: 31; is encoded by the nucleotide sequence of SEQ ID NO: 10, or a nucleotide sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 10; and/or comprises the ABL1 kinase domain, or a portion of the ABL1 kinase domain having kinase activity; wherein the cancer is a lung cancer, such as a lung adenocarcinoma.

In some embodiments, an ABL1 fusion polypeptide of the disclosure is a BMP2K-ABL1 fusion polypeptide, wherein the BMP2K-ABL1 fusion polypeptide: is encoded by an ABL1 fusion nucleic acid molecule comprising in 5′ to 3′ direction, at least a portion of a BMP2K gene fused to at least a portion of an ABL1 gene; comprises a fusion between an amino acid sequence encoded by exon 15, or a portion thereof, of a BMP2K gene and an amino acid sequence encoded by exon 3, or a portion thereof, of an ABL1 gene; is encoded by an ABL1 fusion nucleic acid molecule comprising or resulting from a 5′ breakpoint within chromosomal coordinates chr4: 79800022-79800062 and/or a 3′ breakpoint within chromosomal coordinates chr9: 133730205-133730245; comprises, in the N- to C-terminus direction, an amino acid sequence encoded by exons 1-14 and exon 15, or a portion thereof, of a BMP2K gene, fused to an amino acid sequence encoded by exon 3, or a portion thereof, and exons 4-11 of an ABL1 gene; is encoded by an ABL1 fusion nucleic acid molecule comprising, in the 5′ to 3′ direction, exons 1-14 and exon 15, or a portion thereof, of a BMP2K gene, fused to exon 3, or a portion thereof, and exons 4-11 of an ABL1 gene; and/or comprises the ABL1 kinase domain, or a portion of the ABL1 kinase domain having kinase activity; wherein the cancer is a leukemia, such as a bone marrow leukemia T cell acute (T-ALL).

In some embodiments, an ABL1 fusion nucleic acid molecule of the disclosure comprises a fusion between an ABL1 gene, or a portion thereof, fused to a second gene, or a portion thereof, wherein the ABL1 fusion nucleic acid molecule comprises or results from an ABL1 breakpoint within exon 1 of ABL1. In some embodiments, the ABL1 fusion nucleic acid molecule comprises, in 5′ to 3′ direction, the second gene, or a portion thereof, fused to exon 1 of an ABL1 gene, or a portion thereof. In some embodiments, the ABL1 fusion nucleic acid molecule comprises a fusion between an exon or an intron, or a portion thereof, of the second gene fused to exon 1, or a portion thereof, of an ABL1 gene. In some embodiments, the ABL1 fusion nucleic acid molecule comprises or results from a breakpoint within chromosomal coordinates chr9: 133589786-133589985. In some embodiments, the ABL1 fusion nucleic acid molecule comprises, in 5′ to 3′ direction, one or more exons, or portions thereof, of the second gene, fused to exon 1, or a portion thereof, and exons 2-11 of ABL1. In some embodiments, the second gene is an EXOSC2 gene, and wherein the ABL1 fusion nucleic acid molecule: comprises a fusion between exon 9, or a portion thereof, of an EXOSC2 gene fused to exon 1, or a portion thereof, of an ABL1 gene; comprises or results from a breakpoint within exon 9 of an EXOSC2 gene and a breakpoint within exon 1 of an ABL1 gene; comprises or results from a 5′ breakpoint within chromosomal coordinates chr9: 133579154-133579531 and/or a 3′ breakpoint within chromosomal coordinates chr9: 133589786-133589985; and/or comprises, in 5′ to 3′ direction, exons 1-8 and exon 9, or a portion thereof, of an EXOSC2 gene, fused to exon 1, or a portion thereof, and exons 2-11 of an ABL1 gene. In some embodiments, the cancer is a peritoneum cancer, such as a peritoneum adenocarcinoma.

In some embodiments, an ABL1 fusion polypeptide of the disclosure comprises a fusion between an ABL1 polypeptide, or a portion thereof, fused to a polypeptide encoded by a second gene, or a portion thereof, wherein the ABL1 fusion polypeptide results from an ABL1 breakpoint within exon 1 of an ABL1 gene. In some embodiments, the ABL1 fusion polypeptide comprises, in the N- to C-terminus direction, an amino acid sequence encoded by the second gene, or a portion thereof, fused to an amino acid sequence encoded by exon 1 of an ABL1 gene, or a portion thereof. In some embodiments, the ABL1 fusion polypeptide comprises a fusion between an amino acid sequence encoded by an exon, or a portion thereof, of the second gene fused to an amino acid sequence encoded by exon 1, or a portion thereof, of an ABL1 gene. In some embodiments, the ABL1 fusion polypeptide results from a breakpoint within chromosomal coordinates chr9: 133589786-133589985. In some embodiments, the ABL1 fusion polypeptide comprises, in the N- to C-terminus direction, an amino acid sequence encoded by one or more exons, or portions thereof, of the second gene, fused to an amino acid sequence encoded by exon 1, or a portion thereof, and exons 2-11 of ABL1. In some embodiments, the second gene is an EXOSC2 gene, and wherein the ABL1 fusion polypeptide: comprises a fusion between an amino acid sequence encoded by exon 9, or a portion thereof, of an EXOSC2 gene fused to an amino acid sequence encoded by exon 1, or a portion thereof, of an ABL1 gene; results from a breakpoint within exon 9 of an EXOSC2 gene and a breakpoint within exon 1 of an ABL1 gene; results from a 5′ breakpoint within chromosomal coordinates chr9: 133579154-133579531 and/or a 3′ breakpoint within chromosomal coordinates chr9: 133589786-133589985; and/or comprises, in the N- to C-terminus direction, an amino acid sequence encoded by exons 1-8 and exon 9, or a portion thereof, of an EXOSC2 gene, fused to an amino acid sequence encoded by exon 1, or a portion thereof, and exons 2-11 of an ABL1 gene. In some embodiments, the cancer is a peritoneum cancer, such as a peritoneum adenocarcinoma.

In some embodiments, an ABL1 fusion nucleic acid molecule of the disclosure comprises a fusion between an ABL1 gene, or a portion thereof, fused to a second gene, or a portion thereof, wherein the ABL1 fusion nucleic acid molecule comprises or results from an ABL1 breakpoint within exon 2 of ABL1. In some embodiments, the ABL1 fusion nucleic acid molecule comprises, in 5′ to 3′ direction, the second gene, or a portion thereof, fused to exon 2, or a portion thereof, of an ABL1 gene. In some embodiments, the ABL1 fusion nucleic acid molecule comprises a fusion between an exon or an intron, or a portion thereof, of the second gene fused to exon 2, or a portion thereof, of an ABL1 gene. In some embodiments, the ABL1 fusion nucleic acid molecule comprises or results from a breakpoint within chromosomal coordinates chr9: 133729584-133729766. In some embodiments, the ABL1 fusion nucleic acid molecule comprises, in 5′ to 3′ direction, one or more exons, or portions thereof, of the second gene, fused to exon 2, or a portion thereof, and exons 3-11 of an ABL1 gene. In some embodiments, the second gene is an APOOL gene, wherein the ABL1 fusion nucleic acid molecule: comprises or results from a breakpoint within intron 2 of an APOOL gene, and exon 2 of ABL1; comprises a fusion between exon 2, or a portion thereof, of an APOOL gene fused to exon 2, or a portion thereof, of an ABL1 gene; comprises or results from a 5′ breakpoint within chromosomal coordinates chrX: 84302705-84302904 and/or 3′ breakpoint within chromosomal coordinates chr9: 133729584-133729766; and/or comprises, in 5′ to 3′ direction, exon 1 and exon 2, or a portion thereof of an APOOL gene, fused to exon 2, or a portion thereof, and exons 3-11 of an ABL1 gene. In some embodiments, the cancer is a lung cancer, such as a lung adenocarcinoma.

In some embodiments, an ABL1 fusion polypeptide of the disclosure comprises a fusion between an ABL1 polypeptide, or a portion thereof, fused to a polypeptide encoded by a second gene, or a portion thereof, wherein the ABL1 fusion polypeptide results from an ABL1 breakpoint within exon 2 of ABL1. In some embodiments, the ABL1 fusion polypeptide comprises, in the N- to C-terminus direction, an amino acid sequence encoded by the second gene, or a portion thereof, fused to an amino acid sequence encoded by exon 2, or a portion thereof, of an ABL1 gene. In some embodiments, the ABL1 fusion polypeptide comprises a fusion between an amino acid sequence encoded by an exon, or a portion thereof, of the second gene fused to an amino acid sequence encoded by exon 2, or a portion thereof, of an ABL1 gene. In some embodiments, the ABL1 fusion polypeptide results from a breakpoint within chromosomal coordinates chr9: 133729584-133729766. In some embodiments, the ABL1 fusion polypeptide comprises, in the N- to C-terminus direction, an amino acid sequence encoded by one or more exons, or portions thereof, of the second gene, fused to an amino acid sequence encoded by exon 2, or a portion thereof, and exons 3-11 of an ABL1 gene. In some embodiments, the second gene is an APOOL gene, wherein the ABL1 fusion polypeptide: results from a breakpoint within intron 2 of an APOOL gene, and exon 2 of an ABL1 gene; comprises a fusion between an amino acid sequence encoded by exon 2, or a portion thereof, of an APOOL gene fused to an amino acid sequence encoded by exon 2, or a portion thereof, of an ABL1 gene; results from a 5′ breakpoint within chromosomal coordinates chrX: 84302705-84302904 and/or 3′ breakpoint within chromosomal coordinates chr9: 133729584-133729766; and/or comprises, in the N- to C-terminus direction, an amino acid sequence encoded by exon 1 and exon 2, or a portion thereof of an APOOL gene, fused to an amino acid sequence encoded by exon 2, or a portion thereof, and exons 3-11 of an ABL1 gene. In some embodiments, the cancer is a lung cancer, such as a lung adenocarcinoma.

In some embodiments, an ABL1 fusion nucleic acid molecule of the disclosure comprises a fusion between an ABL1 gene, or a portion thereof, fused to a second gene, or a portion thereof, wherein the ABL1 fusion nucleic acid molecule comprises or results from an ABL1 breakpoint within exon 3 of ABL1. In some embodiments, the ABL1 fusion nucleic acid molecule comprises, in 5′ to 3′ direction, the second gene, or a portion thereof, fused to exon 3, or a portion thereof, of an ABL1 gene. In some embodiments, the ABL1 fusion nucleic acid molecule comprises a fusion between an exon or an intron, or a portion thereof, of the second gene fused to exon 3, or a portion thereof, of an ABL1 gene. In some embodiments, the ABL1 fusion nucleic acid molecule comprises or results from a breakpoint within chromosomal coordinates chr9: 133730205-133730245, chr9: 133730195-133730235, chr9: 133730203-133730243, chr9: 133730234-133730274 or chr9: 133730218-133730258. In some embodiments, the ABL1 fusion nucleic acid molecule comprises, in 5′ to 3′ direction, one or more exons, or portions thereof, of the second gene, fused to exon 3, or a portion thereof, and exons 4-11 of an ABL1 gene. In some embodiments, the cancer is a bone marrow leukemia, optionally wherein the bone marrow leukemia is a bone marrow leukemia lymphocytic acute (ALL), a bone marrow leukemia B cell acute (B-ALL), or a bone marrow leukemia T cell acute (T-ALL). In some embodiments, the second gene is a NUP214 gene, wherein the ABL1 fusion nucleic acid molecule: comprises a fusion between exon 34, or a portion thereof, of a NUP214 gene fused to exon 3, or a portion thereof, of an ABL1 gene; comprises or results from a breakpoint within intron 34 of a NUP214 gene and a breakpoint within exon 3 of an ABL1 gene; comprises or results from: a 5′ breakpoint within chromosomal coordinates chr9: 134106080-134106120 and/or a 3′ breakpoint within chromosomal coordinates chr9: 133730195-133730235, a 5′ breakpoint within chromosomal coordinates chr9: 134106030-134106070 and/or a 3′ breakpoint within chromosomal coordinates chr9: 133730218-133730258, a 5′ breakpoint within chromosomal coordinates chr9: 134106046-134106086 and/or a 3′ breakpoint within chromosomal coordinates chr9: 133730203-133730243, or a 5′ breakpoint within chromosomal coordinates chr9: 134106028-134106068 and/or a 3′ breakpoint within chromosomal coordinates chr9: 133730234-133730274; and/or comprises, in the 5′ to 3′ direction, exons 1-33 and exon 34, or a portion thereof, of a NUP214 gene, fused to exon 3, or a portion thereof, and exons 4-11 of an ABL1 gene; optionally, wherein the cancer is a bone marrow leukemia, and optionally wherein the bone marrow leukemia is a bone marrow leukemia B cell acute (B-ALL) or a bone marrow leukemia lymphocytic acute (ALL). In some embodiments, the second gene is a BMP2K gene, wherein the ABL1 fusion nucleic acid molecule: comprises a fusion between exon 15, or a portion thereof, of a BMP2K gene fused to exon 3, or a portion thereof, of an ABL1 gene; comprises or results from a 5′ breakpoint within chromosomal coordinates chr4: 79800022-79800062 and/or a 3′ breakpoint within chromosomal coordinates chr9: 133730205-133730245; and/or comprises, in 5′ to 3′ direction, exons 1-14 and exon 15, or a portion thereof, of a BMP2K gene, fused to exon 3, or a portion thereof, and exons 4-11 of an ABL1 gene; optionally, wherein the cancer is a bone marrow leukemia, and optionally wherein the bone marrow leukemia is a bone marrow leukemia T cell acute (T-ALL). In some embodiments, the second gene is a BCR gene, wherein the ABL1 fusion nucleic acid molecule: comprises a fusion between exon 1, or a portion thereof, of a BCR gene fused to exon 3, or a portion thereof, of an ABL1 gene; comprises or results from a 5′ breakpoint within chromosomal coordinates chr22: 23524272-23524312 and/or a 3′ breakpoint within chromosomal coordinates chr9: 133730218-133730258; and/or comprises, in 5′ to 3′ direction, exon 1, or a portion thereof of a BCR gene, fused to exon 3, or a portion thereof, and exons 4-11 of an ABL1 gene; optionally, wherein the cancer is a bone marrow leukemia, and optionally wherein the bone marrow leukemia is a bone marrow leukemia lymphocytic acute (ALL).

In some embodiments, an ABL1 fusion polypeptide of the disclosure comprises a fusion between an ABL1 polypeptide, or a portion thereof, fused to a polypeptide encoded by a second gene, or a portion thereof, wherein the ABL1 fusion polypeptide results from an ABL1 breakpoint within exon 3 of an ABL1 gene. In some embodiments, the ABL1 fusion polypeptide comprises, in the N- to C-terminus direction, an amino acid sequence encoded by the second gene, or a portion thereof, fused to an amino acid sequence encoded by exon 3, or a portion thereof, of an ABL1 gene. In some embodiments, the ABL1 fusion polypeptide comprises a fusion between an amino acid sequence encoded by an exon, or a portion thereof, of the second gene fused to an amino acid sequence encoded by exon 3, or a portion thereof, of an ABL1 gene. In some embodiments, the ABL1 fusion polypeptide results from a breakpoint within chromosomal coordinates chr9: 133730205-133730245, chr9: 133730195-133730235, chr9: 133730203-133730243, chr9: 133730234-133730274 or chr9: 133730218-133730258. In some embodiments, the ABL1 fusion polypeptide comprises, in the N- to C-terminus direction, an amino acid sequence encoded by one or more exons, or portions thereof, of the second gene, fused to an amino acid sequence encoded by exon 3, or a portion thereof, and exons 4-11 of an ABL1 gene; optionally wherein the cancer is a bone marrow leukemia, optionally wherein the bone marrow leukemia is a bone marrow leukemia lymphocytic acute (ALL), a bone marrow leukemia B cell acute (B-ALL), or a bone marrow leukemia T cell acute (T-ALL). In some embodiments, the second gene is a NUP214 gene, wherein the ABL1 fusion polypeptide: comprises a fusion between an amino acid sequence encoded by exon 34, or a portion thereof, of a NUP214 gene fused to an amino acid sequence encoded by exon 3, or a portion thereof, of an ABL1 gene; results from a breakpoint within intron 34 of a NUP214 gene and a breakpoint within exon 3 of an ABL1 gene; results from: a 5′ breakpoint within chromosomal coordinates chr9: 134106080-134106120 and/or a 3′ breakpoint within chromosomal coordinates chr9: 133730195-133730235, a 5′ breakpoint within chromosomal coordinates chr9: 134106030-134106070 and/or a 3′ breakpoint within chromosomal coordinates chr9: 133730218-133730258, a 5′ breakpoint within chromosomal coordinates chr9: 134106046-134106086 and/or a 3′ breakpoint within chromosomal coordinates chr9: 133730203-133730243, or a 5′ breakpoint within chromosomal coordinates chr9: 134106028-134106068 and/or a 3′ breakpoint within chromosomal coordinates chr9: 133730234-133730274; and/or comprises, in the N- to C-terminus direction, an amino acid sequence encoded by exons 1-33 and exon 34, or a portion thereof, of a NUP214 gene, fused to an amino acid sequence encoded by exon 3, or a portion thereof, and exons 4-11 of an ABL1 gene; optionally, wherein the cancer is a bone marrow leukemia, and optionally wherein the bone marrow leukemia is a bone marrow leukemia B cell acute (B-ALL) or a bone marrow leukemia lymphocytic acute (ALL). In some embodiments, the second gene is a BMP2K gene, wherein the ABL1 fusion polypeptide: comprises a fusion between an amino acid sequence encoded by exon 15, or a portion thereof, of a BMP2K gene fused to an amino acid sequence encoded by exon 3, or a portion thereof, of an ABL1 gene; results from a 5′ breakpoint within chromosomal coordinates chr4: 79800022-79800062 and/or a 3′ breakpoint within chromosomal coordinates chr9: 133730205-133730245; and/or comprises, in the N- to C-terminus direction, an amino acid sequence encoded by exons 1-14 and exon 15, or a portion thereof, of a BMP2K gene, fused to an amino acid sequence encoded by exon 3, or a portion thereof, and exons 4-11 of an ABL1 gene; optionally, wherein the cancer is a bone marrow leukemia, and optionally wherein the bone marrow leukemia is a bone marrow leukemia T cell acute (T-ALL). In some embodiments, the second gene is a BCR gene, wherein the ABL1 fusion polypeptide: comprises a fusion between an amino acid sequence encoded by exon 1, or a portion thereof, of a BCR gene fused to an amino acid sequence encoded by exon 3, or a portion thereof, of an ABL1 gene; results from a 5′ breakpoint within chromosomal coordinates chr22: 23524272-23524312 and/or a 3′ breakpoint within chromosomal coordinates chr9: 133730218-133730258; and/or comprises, in the N- to C-terminus direction, an amino acid sequence encoded by exon 1, or a portion thereof, of a BCR gene, fused to an amino acid sequence encoded by exon 3, or a portion thereof, and exons 4-11 of an ABL1 gene; optionally, wherein the cancer is a bone marrow leukemia, and optionally wherein the bone marrow leukemia is a bone marrow leukemia lymphocytic acute (ALL).

In some embodiments, an ABL1 fusion nucleic acid molecule of the disclosure comprises a fusion between an ABL1 gene, or a portion thereof, fused to a second gene, or a portion thereof, wherein the ABL1 fusion nucleic acid molecule comprises or results from an ABL1 breakpoint within exon 4 of ABL1. In some embodiments, the ABL1 fusion nucleic acid molecule comprises, in 5′ to 3′ direction, the second gene, or a portion thereof, fused to exon 4, or a portion thereof, of an ABL1 gene. In some embodiments, the ABL1 fusion nucleic acid molecule comprises a fusion between an exon or an intron, or a portion thereof, of the second gene fused to exon 4, or a portion thereof, of an ABL1 gene. In some embodiments, the ABL1 fusion nucleic acid molecule comprises or results from a breakpoint within chromosomal coordinates chr9: 133738173-133738213, chr9: 133738222-133738262, or chr9: 133738214-133738254. In some embodiments, the ABL1 fusion nucleic acid molecule comprises, in 5′ to 3′ direction, one or more exons, or portions thereof, of the second gene, fused to exon 4, or a portion thereof, and exons 5-11 of an ABL1 gene; optionally wherein the cancer is a leukemia, optionally wherein the leukemia is a bone marrow leukemia B cell acute (B-ALL), a B-lymphoblastic leukemia-lymphoma (B-ALL), or a bone marrow leukemia lymphocytic chronic (CLL). In some embodiments, the second gene is an RCSD1 gene, wherein the ABL1 fusion nucleic acid molecule: comprises a fusion between exon 4, or a portion thereof, of an RCSD1 gene fused to exon 4, or a portion thereof, of an ABL1 gene; comprises or results from a 5′ breakpoint within chromosomal coordinates chr1: 167654668-167654708 and/or a 3′ breakpoint within chromosomal coordinates chr9: 133738173-133738213; comprises, in 5′ to 3′ direction, exons 1-3 and exon 4, or a portion thereof, of an RCSD1 gene, fused to exon 4, or a portion thereof, and exons 5-11 of an ABL1 gene; comprises the nucleotide sequence of SEQ ID NO: 21, or a nucleotide sequence having at least about 70% homology thereto; and/or encodes an ABL1 fusion polypeptide comprising the amino acid sequence of SEQ ID NO: 42, or an amino acid sequence having at least about 70% homology thereto; optionally, wherein the cancer is a bone marrow leukemia, and optionally wherein the bone marrow leukemia is a bone marrow leukemia lymphocytic chronic (CLL). In some embodiments, the second gene is an SNX2 gene, wherein the ABL1 fusion nucleic acid molecule: comprises a fusion between exon 3, or a portion thereof, of an SNX2 gene fused to exon 4, or a portion thereof, of an ABL1 gene; comprises or results from: a 5′ breakpoint within chromosomal coordinates chr5: 122135451-122135491 and/or a 3′ breakpoint within chromosomal coordinates chr9: 133738222-133738262, or a 5′ breakpoint within chromosomal coordinates chr5: 122135445-122135485 and/or a 3′ breakpoint within chromosomal coordinates chr9: 133738214-133738254; and/or comprises, in 5′ to 3′ direction, exons 1-2 and exon 3, or a portion thereof of an SNX2 gene, fused to exon 4, or a portion thereof, and exons 5-11 of an ABL1 gene; optionally wherein the cancer is a leukemia, optionally wherein the leukemia is a bone marrow leukemia B cell acute (B-ALL) or a B-lymphoblastic leukemia-lymphoma (B-ALL).

In some embodiments, an ABL1 fusion polypeptide of the disclosure comprises a fusion between an ABL1 polypeptide, or a portion thereof, fused to a polypeptide encoded by a second gene, or a portion thereof, wherein the ABL1 fusion polypeptide comprises or results from an ABL1 breakpoint within exon 4 of ABL1. In some embodiments, the ABL1 fusion polypeptide comprises, in the N- to C-terminus direction, an amino acid sequence encoded by the second gene, or a portion thereof, fused to an amino acid sequence encoded by exon 4, or a portion thereof, of an ABL1 gene. In some embodiments, the ABL1 fusion polypeptide comprises a fusion between an amino acid sequence encoded by an exon, or a portion thereof, of the second gene fused to an amino acid sequence encoded by exon 4, or a portion thereof, of an ABL1 gene. In some embodiments, the ABL1 fusion polypeptide results from a breakpoint within chromosomal coordinates chr9: 133738173-133738213, chr9: 133738222-133738262, or chr9: 133738214-133738254. In some embodiments, the ABL1 fusion polypeptide comprises, in the N- to C-terminus direction, an amino acid sequence encoded by one or more exons, or portions thereof, of the second gene, fused to an amino acid sequence encoded by exon 4, or a portion thereof, and exons 5-11 of ABL1; optionally wherein the cancer is a leukemia, optionally wherein the leukemia is a bone marrow leukemia B cell acute (B-ALL), a B-lymphoblastic leukemia-lymphoma (B-ALL), or a bone marrow leukemia lymphocytic chronic (CLL). In some embodiments, the second gene is an RCSD1 gene, and wherein the ABL1 fusion polypeptide: comprises a fusion between an amino acid sequence encoded by exon 4, or a portion thereof, of an RCSD1 gene fused to an amino acid sequence encoded by exon 4, or a portion thereof, of an ABL1 gene; results from a 5′ breakpoint within chromosomal coordinates chr1: 167654668-167654708 and/or a 3′ breakpoint within chromosomal coordinates chr9: 133738173-133738213; comprises, in the N- to C-terminus direction, an amino acid sequence encoded by exons 1-3 and exon 4, or a portion thereof, of an RCSD1 gene, fused to an amino acid sequence encoded by exon 4, or a portion thereof, and exons 5-11 of an ABL1 gene; is encoded by the nucleotide sequence of SEQ ID NO: 21, or a nucleotide sequence having at least about 70% homology thereto; and/or comprises the amino acid sequence of SEQ ID NO: 42, or an amino acid sequence having at least about 70% homology thereto; optionally, wherein the cancer is a bone marrow leukemia, and optionally wherein the bone marrow leukemia is a bone marrow leukemia lymphocytic chronic (CLL). In some embodiments, the second gene is an SNX2 gene, and wherein the ABL1 fusion polypeptide: comprises a fusion between an amino acid sequence encoded by exon 3, or a portion thereof, of an SNX2 gene fused to an amino acid sequence encoded by exon 4, or a portion thereof, of an ABL1 gene; results from: a 5′ breakpoint within chromosomal coordinates chr5: 122135451-122135491 and/or a 3′ breakpoint within chromosomal coordinates chr9: 133738222-133738262, or a 5′ breakpoint within chromosomal coordinates chr5: 122135445-122135485 and/or a 3′ breakpoint within chromosomal coordinates chr9: 133738214-133738254; and/or comprises, in the N- to C-terminus direction, an amino acid sequence encoded by exons 1-2 and exon 3, or a portion thereof, of an SNX2 gene, fused to an amino acid sequence encoded by exon 4, or a portion thereof, and exons 5-11 of an ABL1 gene; optionally wherein the cancer is a leukemia, optionally wherein the leukemia is a bone marrow leukemia B cell acute (B-ALL) or a B-lymphoblastic leukemia-lymphoma (B-ALL).

In some embodiments, an ABL1 fusion nucleic acid molecule of the disclosure comprises a fusion between an ABL1 gene, or a portion thereof, fused to a second gene, or a portion thereof, wherein the ABL1 fusion nucleic acid molecule comprises or results from an ABL1 breakpoint within exon 10 of ABL1. In some embodiments, the ABL1 fusion nucleic acid molecule comprises, in 5′ to 3′ direction, exon 10, or a portion thereof, of an ABL1 gene fused to the second gene, or a portion thereof. In some embodiments, the ABL1 fusion nucleic acid molecule comprises a fusion between an exon or an intron, or a portion thereof, of the second gene fused to exon 10, or a portion thereof, of an ABL1 gene. In some embodiments, the ABL1 fusion nucleic acid molecule comprises or results from a breakpoint within chromosomal coordinates chr9: 133755953-133755993. In some embodiments, the ABL1 fusion nucleic acid molecule comprises, in 5′ to 3′ direction, exons 1-9 and exon 10, or a portion thereof, of an ABL1 gene fused to one or more exons, or portions thereof, of the second gene; optionally wherein the cancer is soft tissue sarcoma, and optionally wherein the soft tissue sarcoma is not otherwise specified (NOS). In some embodiments, the second gene is a DNAH2 gene, wherein the ABL1 fusion nucleic acid molecule: (a) comprises a fusion between exon 10, or a portion thereof, of an ABL1 gene fused to exon 4, or a portion thereof, of a DNAH2 gene; (b) comprises or results from a 5′ breakpoint within chromosomal coordinates chr9: 133755953-133755993 and/or a 3′ breakpoint within chromosomal coordinates chr17: 7636399-7636439; and/or (c) comprises, in 5′ to 3′ direction, exons 1-9 and exon 10, or a portion thereof, of an ABL1 gene fused to exon 4, or a portion thereof, and exons 5-85 of a DNAH2 gene; optionally, wherein the cancer is a soft tissue sarcoma, and optionally wherein the soft tissue sarcoma is not otherwise specified (NOS).

In some embodiments, an ABL1 fusion polypeptide of the disclosure comprises a fusion between an ABL1 polypeptide, or a portion thereof, fused to a polypeptide encoded by a second gene, or a portion thereof, wherein the ABL1 fusion polypeptide results from an ABL1 breakpoint within exon 10 of an ABL1 gene. In some embodiments, the ABL1 fusion polypeptide comprises, in the N- to C-terminus direction, an amino acid sequence encoded by exon 10, or a portion thereof, of an ABL1 gene fused to an amino acid sequence encoded by the second gene, or a portion thereof. In some embodiments, the ABL1 fusion polypeptide comprises a fusion between an amino acid sequence encoded by an exon, or a portion thereof, of the second gene fused to an amino acid sequence encoded by exon 10, or a portion thereof, of an ABL1 gene. In some embodiments, the ABL1 fusion polypeptide results from a breakpoint within chromosomal coordinates chr9: 133755953-133755993. In some embodiments, the ABL1 fusion polypeptide comprises, in the N- to C-terminus direction, an amino acid sequence encoded by exons 1-9 and exon 10, or a portion thereof, of an ABL1 gene fused to an amino acid sequence encoded by one or more exons, or portions thereof, of the second gene; optionally wherein the cancer is soft tissue sarcoma, and optionally wherein the soft tissue sarcoma is not otherwise specified (NOS). In some embodiments, the second gene is a DNAH2 gene, wherein the ABL1 fusion polypeptide: comprises a fusion between an amino acid sequence encoded by exon 10, or a portion thereof, of an ABL1 gene fused to an amino acid sequence encoded by exon 4, or a portion thereof, of a DNAH2 gene; results from a 5′ breakpoint within chromosomal coordinates chr9: 133755953-133755993 and/or a 3′ breakpoint within chromosomal coordinates chr17: 7636399-7636439; and/or comprises, in the N- to C-terminus direction, an amino acid sequence encoded by exons 1-9 and exon 10, or a portion thereof, of an ABL1 gene fused to an amino acid sequence encoded by exon 4, or a portion thereof, and exons 5-85 of a DNAH2 gene; optionally, wherein the cancer is a soft tissue sarcoma, and optionally wherein the soft tissue sarcoma is not otherwise specified (NOS).

In some embodiments, an ABL1 fusion nucleic acid molecule of the disclosure comprises a fusion between an ABL1 gene, or a portion thereof, fused to a second gene, or a portion thereof, wherein the ABL1 fusion nucleic acid molecule comprises or results from an ABL1 breakpoint within intron 4 of an ABL1 gene. In some embodiments, the ABL1 fusion nucleic acid molecule comprises, in 5′ to 3′ direction, the second gene, or a portion thereof, fused to exon 5, or a portion thereof, of an ABL1 gene. In some embodiments, the ABL1 fusion nucleic acid molecule comprises a fusion between an exon or an intron of the second gene, or a portion thereof, fused to intron 4, or a portion thereof, of an ABL1 gene. In some embodiments, the ABL1 fusion nucleic acid molecule comprises or results from a breakpoint within chromosomal coordinates chr9: 133747364-133747762. In some embodiments, the ABL1 fusion nucleic acid molecule comprises, in 5′ to 3′ direction, one or more exons, or portions thereof, of the second gene, fused to exon 5, or a portion thereof, and exons 6-11 of an ABL1 gene; optionally wherein the cancer is an ovary epithelial carcinoma, and optionally wherein the ovary epithelial carcinoma is not otherwise specified (NOS). In some embodiments, the second gene is a MED27 gene, wherein the ABL1 fusion nucleic acid molecule: comprises a fusion between exon 4, or a portion thereof, of a MED27 gene fused to exon 5, or a portion thereof, of an ABL1 gene; comprises or results from a breakpoint within intron 4 of a MED27 gene and a breakpoint within intron 4 of an ABL1 gene; comprises or results from a 5′ breakpoint within chromosomal coordinates chr9: 134774423-134776006 and/or a 3′ breakpoint within chromosomal coordinates chr9: 133747364-133747762; comprises, in 5′ to 3′ direction, exons 1-3 and exon 4, or a portion thereof of a MED27 gene, fused to exon 5, or a portion thereof, and exons 6-11 of an ABL1 gene; comprises the nucleotide sequence of SEQ ID NO: 3, or a nucleotide sequence having at least about 70% homology thereto; and/or encodes an ABL1 fusion polypeptide comprising the amino acid sequence of SEQ ID NO: 24, or an amino acid sequence having at least about 70% homology thereto; optionally, wherein the cancer is an ovary epithelial carcinoma, and optionally wherein the ovary epithelial carcinoma is not otherwise specified (NOS).

In some embodiments, an ABL1 fusion polypeptide of the disclosure comprises a fusion between an ABL1 polypeptide, or a portion thereof, fused to a polypeptide encoded by a second gene, or a portion thereof, wherein the ABL1 fusion polypeptide results from an ABL1 breakpoint within intron 4 of an ABL1 gene. In some embodiments, the ABL1 fusion polypeptide comprises, in the N- to C-terminus direction, an amino acid sequence encoded by the second gene, or a portion thereof, fused to an amino acid sequence encoded by exon 5, or a portion thereof, of an ABL1 gene. In some embodiments, the ABL1 fusion polypeptide comprises a fusion between an amino acid sequence encoded by an exon of the second gene, or a portion thereof, fused to an amino acid sequence encoded by exon 5, or a portion thereof, of an ABL1 gene. In some embodiments, the ABL1 fusion polypeptide results from a breakpoint within chromosomal coordinates chr9: 133747364-133747762. In some embodiments, the ABL1 fusion polypeptide comprises, in the N- to C-terminus direction, an amino acid sequence encoded by one or more exons, or portions thereof, of the second gene, fused to an amino acid sequence encoded by exon 5, or a portion thereof, and exons 6-11 of an ABL1 gene; optionally wherein the cancer is an ovary epithelial carcinoma, and optionally wherein the ovary epithelial carcinoma is not otherwise specified (NOS). In some embodiments, the second gene is a MED27 gene, wherein the ABL1 fusion polypeptide: comprises a fusion between an amino acid sequence encoded by exon 4, or a portion thereof, of a MED27 gene fused to an amino acid sequence encoded by exon 5, or a portion thereof, of an ABL1 gene; results from a breakpoint within intron 4 of a MED27 gene and a breakpoint within intron 4 of an ABL1 gene; results from a 5′ breakpoint within chromosomal coordinates chr9: 134774423-134776006 and/or a 3′ breakpoint within chromosomal coordinates chr9: 133747364-133747762; comprises, in the N- to C-terminus direction, an amino acid sequence encoded by exons 1-3 and exon 4, or a portion thereof, of a MED27 gene, fused to an amino acid sequence encoded by exon 5, or a portion thereof, and exons 6-11 of an ABL1 gene; is encoded by the nucleotide sequence of SEQ ID NO: 3, or a nucleotide sequence having at least about 70% homology thereto; and/or comprises the amino acid sequence of SEQ ID NO: 24, or an amino acid sequence having at least about 70% homology thereto; optionally, wherein the cancer is an ovary epithelial carcinoma, and optionally wherein the ovary epithelial carcinoma is not otherwise specified (NOS).

In some embodiments, an ABL1 fusion nucleic acid molecule of the disclosure comprises a fusion between an ABL1 gene, or a portion thereof, fused to a second gene, or a portion thereof, wherein the ABL1 fusion nucleic acid molecule comprises or results from an ABL1 breakpoint within intron 5 of an ABL1 gene. In some embodiments, the ABL1 fusion nucleic acid molecule comprises, in 5′ to 3′ direction, the second gene, or a portion thereof, fused to exon 6, or a portion thereof, of an ABL1 gene. In some embodiments, the ABL1 fusion nucleic acid molecule comprises a fusion between an exon or an intron, or a portion thereof, of the second gene fused to intron 5, or a portion thereof, of an ABL1 gene. In some embodiments, the ABL1 fusion nucleic acid molecule comprises or results from a breakpoint within chromosomal coordinates chr9: 133748211-133748501. In some embodiments, the ABL1 fusion nucleic acid molecule comprises, in 5′ to 3′ direction, one or more exons, or portions thereof, of the second gene, fused to exon 6, or a portion thereof, and exons 7-11 of an ABL1 gene; optionally wherein the cancer is a testis germ cell tumor, and optionally wherein the testis germ cell tumor is a mixed testis germ cell tumor. In some embodiments, the second gene is an SLC27A4 gene, wherein the ABL1 fusion nucleic acid molecule: comprises a fusion between exon 12, or a portion thereof, of a SLC27A4 gene fused to exon 6, or a portion thereof, of an ABL1 gene; comprises or results from a breakpoint within intron 12 of a SLC27A4 gene and a breakpoint within intron 5 of an ABL1 gene; comprises or results from a 5′ breakpoint within chromosomal coordinates chr9: 131119603-131119801 and/or a 3′ breakpoint within chromosomal coordinates chr9: 133748211-133748501; comprises, in 5′ to 3′ direction, exons 1-11 and exon 12, or a portion thereof, of an SLC27A4 gene, fused to exon 6, or a portion thereof, and exons 7-11 of an ABL1 gene; comprises the nucleotide sequence of SEQ ID NO: 5, or a nucleotide sequence having at least about 70% homology thereto; and/or encodes an ABL1 fusion polypeptide comprising the amino acid sequence of SEQ ID NO: 26, or an amino acid sequence having at least about 70% homology thereto; optionally, wherein the cancer is a testis germ cell tumor, wherein the testis germ cell tumor is a mixed testis germ cell tumor.

In some embodiments, an ABL1 fusion polypeptide of the disclosure comprises a fusion between an ABL1 polypeptide, or a portion thereof, fused to a polypeptide encoded by a second gene, or a portion thereof, wherein the ABL1 fusion polypeptide results from an ABL1 breakpoint within intron 5 of ABL1. In some embodiments, the ABL1 fusion polypeptide comprises, in the N- to C-terminus direction, an amino acid sequence encoded by the second gene, or a portion thereof, fused to an amino acid sequence encoded by exon 6, or a portion thereof, of an ABL1 gene. In some embodiments, the ABL1 fusion polypeptide comprises a fusion between an amino acid sequence encoded by an exon, or a portion thereof, of the second gene fused to an amino acid sequence encoded by exon 6, or a portion thereof, of an ABL1 gene. In some embodiments, the ABL1 fusion polypeptide results from a breakpoint within chromosomal coordinates chr9: 133748211-133748501. In some embodiments, the ABL1 fusion polypeptide comprises, in the N- to C-terminus direction, an amino acid sequence encoded by one or more exons, or portions thereof, of the second gene, fused to an amino acid sequence encoded by exon 6, or a portion thereof, and exons 7-11 of an ABL1 gene; optionally wherein the cancer is a testis germ cell tumor, and optionally wherein the testis germ cell tumor is a mixed testis germ cell tumor. In some embodiments, the second gene is an SLC27A4 gene, wherein the ABL1 fusion polypeptide: comprises a fusion between an amino acid sequence encoded by exon 12, or a portion thereof, of a SLC27A4 gene fused to an amino acid sequence encoded by exon 6, or a portion thereof, of an ABL1 gene; results from a breakpoint within intron 12 of a SLC27A4 gene and a breakpoint within intron 5 of an ABL1 gene; results from a 5′ breakpoint within chromosomal coordinates chr9: 131119603-131119801 and/or a 3′ breakpoint within chromosomal coordinates chr9: 133748211-133748501; comprises, in the N- to C-terminus direction, an amino acid sequence encoded by exons 1-11 and exon 12, or a portion thereof, of an SLC27A4 gene, fused to an amino acid sequence encoded by exon 6, or a portion thereof, and exons 7-11 of an ABL1 gene; is encoded by the nucleotide sequence of SEQ ID NO: 5, or a nucleotide sequence having at least about 70% homology thereto; and/or comprises the amino acid sequence of SEQ ID NO: 26, or an amino acid sequence having at least about 70% homology thereto; optionally, wherein the cancer is a testis germ cell tumor, wherein the testis germ cell tumor is a mixed testis germ cell tumor.

In some embodiments of any of the methods provided herein, the sample is a sample described below. In some embodiments, the sample is obtained from the individual or from the cancer. In some embodiments, the methods further comprise obtaining the sample, e.g., from the individual or from the cancer. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, cerebrospinal fluid, sputum, stool, urine, or saliva. In some embodiments, the sample comprises cells and/or nucleic acids from the cancer. In some embodiments, the sample comprises mRNA, DNA, circulating tumor DNA (ctDNA), cell-free DNA, or cell-free RNA from the cancer. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the ABL1 fusion nucleic acid molecule or ABL1 fusion polypeptide is detected in a tissue biopsy sample, in a liquid biopsy sample, or in both a tissue biopsy sample and a liquid biopsy sample, from the individual.

### B. Detection of ABL1 Fusion Nucleic Acid Molecules and ABL1 Fusion Polypeptides

Certain aspects of the present disclosure relate to detection of an ABL1 fusion nucleic acid molecule of the disclosure, e.g., in a patient sample. In some embodiments, the ABL1 fusion nucleic acid molecule is detected in vitro.

Other aspects of the present disclosure relate to detection of an ABL1 fusion polypeptide of the disclosure, e.g., an ABL1 fusion polypeptide encoded by an ABL1 fusion nucleic acid molecule of the disclosure, e.g., in a patient sample. In some embodiments, the ABL1 fusion polypeptide is detected in vitro.

### (i) Detection of ABL1 Fusion Nucleic Acid Molecules

Methods for detecting an ABL1 fusion nucleic acid molecule of the disclosure are known in the art. For example, in some embodiments, an ABL1 fusion nucleic acid molecule is detected by sequencing part or all of a gene involved in the fusion nucleic acid molecule, e.g., an ABL1 gene, and/or a corresponding fusion partner gene described herein (e.g., any of BMP2K, EHMT1, MED27, RFFL, SLC27A4, BCR, NUP214, AIF1L, RCSD1, APOOL, DNAH2, RHEX/C1orf186, RALGPS1, TSC1, TSPAN18, ZNF804B, EXOSC2, or SNX2), by next-generation or other sequencing of DNA, RNA, or cDNA. In some embodiments, an ABL1 fusion nucleic acid molecule of the disclosure is detected by PCR amplification of DNA, RNA, or cDNA. In some embodiments, an ABL1 fusion nucleic acid molecule of the disclosure is detected by in situ hybridization using one or more polynucleotides that hybridize to a locus involved in the fusion nucleic acid molecule, e.g., an ABL1 locus, and/or a corresponding fusion partner gene locus described herein (e.g., any of BMP2K, EHMT1, MED27, RFFL, SLC27A4, BCR, NUP214, AIF1L, RCSD1, APOOL, DNAH2, RHEX/C1orf186, RALGPS1, TSC1, TSPAN18, ZNF804B, EXOSC2, or SNX2), e.g., using fluorescence in situ hybridization (FISH). In some embodiments, an ABL1 fusion nucleic acid molecule of the disclosure is detected in a cancer or tumor cell, e.g., using tumor tissue, such as from a tumor biopsy or other tumor specimen; in a circulating cancer or tumor cell, e.g., using a liquid biopsy, such as from blood, plasma, cerebrospinal fluid, sputum, stool, urine, or saliva; or in circulating tumor DNA (ctDNA), e.g., using a liquid biopsy, such as from blood, plasma, cerebrospinal fluid, sputum, stool, urine, or saliva.

Exemplary and non-limiting methods for detecting an ABL1 fusion nucleic acid molecule of the disclosure are provided below.

In some embodiments, an ABL1 fusion nucleic acid molecule of the disclosure is detected using any suitable method known in the art, such as a nucleic acid hybridization assay, an amplification-based assay (e.g., polymerase chain reaction, PCR), a PCR-RFLP assay, real-time PCR, sequencing (e.g., Sanger sequencing or next-generation sequencing), a screening analysis (e.g., using karyotype methods), fluorescence in situ hybridization (FISH), break away FISH, spectral karyotyping, multiplex-FISH, comparative genomic hybridization, in situ hybridization, single specific primer-polymerase chain reaction (SSP-PCR), high performance liquid chromatography (HPLC), or mass-spectrometric genotyping. Methods of analyzing samples, e.g., to detect a nucleic acid molecule, are described in U.S. Pat. No. 9,340,830 and in WO2012092426A1, which are hereby incorporated by reference in their entirety. In some embodiments, an ABL1 fusion nucleic acid molecule of the disclosure is detected by sequencing. In some embodiments, the sequencing comprises a massively parallel sequencing (MPS) technique, whole genome sequencing (WGS), whole exome sequencing, targeted sequencing, direct sequencing, or a Sanger sequencing technique. In some embodiments, the massively parallel sequencing (MPS) technique comprises next-generation sequencing (NGS).

In some embodiments, an ABL1 fusion nucleic acid molecule of the disclosure is detected using an in situ hybridization method, such as a fluorescence in situ hybridization (FISH) method.

In some embodiments, FISH analysis is used to identify the chromosomal rearrangement resulting in an ABL1 fusion nucleic acid molecule as described herein. In some embodiments, FISH analysis is used to identify an RNA molecule comprising or encoding an ABL1 fusion nucleic acid molecule of the disclosure. Methods for performing FISH are known in the art and can be used in nearly any type of tissue. In FISH analysis, nucleic acid probes which are detectably labeled, e.g. fluorescently labeled, are allowed to bind to specific regions of DNA, e.g., a chromosome, or an RNA, e.g., an mRNA, and then examined, e.g., through a microscope. See, for example, U.S. Pat. No. 5,776,688. DNA or RNA molecules are first fixed onto a slide, the labeled probe is then hybridized to the DNA or RNA molecules, and then visualization is achieved, e.g., using enzyme-linked label-based detection methods known in the art. Generally, the resolution of FISH analysis is on the order of detection of 60 to 100000 nucleotides, e.g., 60 base pairs (bp) up to 100 kilobase pairs of DNA. Nucleic acid probes used in FISH analysis comprise single stranded nucleic acids. Such probes are typically at least about 50 nucleotides in length. In some embodiments, probes comprise about 100 to about 500 nucleotides. Probes that hybridize with centromeric DNA and locus-specific DNA or RNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA or other sources of nucleic acids through standard techniques. Examples of probes, labeling and hybridization methods are known in the art.

Several variations of FISH methods are known in the art and are suitable for use according to the methods of the disclosure, including single-molecule RNA FISH, Fiber FISH, Q-FISH, Flow-FISH, MA-FISH, break-away FISH, hybrid fusion-FISH, and multi-fluor FISH or mFISH. In some embodiments, “break-away FISH” is used in the methods provided herein. In break-away FISH, at least one probe targeting a fusion junction or breakpoint and at least one probe targeting an individual gene of the fusion, e.g., at one or more exons and or introns of the gene, are utilized. In normal cells (i.e., cells not having a fusion nucleic acid molecule described herein), both probes are observed (or a secondary color is observed due to the close proximity of the two genes of the gene fusion); and in cells having a fusion nucleic acid molecule described herein, only a single gene probe is observed due to the presence of a rearrangement resulting in the fusion nucleic acid molecule.

In some embodiments, an ABL1 fusion nucleic acid molecule of the disclosure is detected using an array-based method, such as array-based comparative genomic hybridization (CGH) methods. In array-based CGH methods, a first sample of nucleic acids (e.g., from a sample, such as from a tumor, or a tissue or liquid biopsy) is labeled with a first label, while a second sample of nucleic acids (e.g., a control, such as from a healthy cell/tissue) is labeled with a second label. In some embodiments, equal quantities of the two samples are mixed and co-hybridized to a DNA microarray of several thousand evenly spaced cloned DNA fragments or oligonucleotides, which have been spotted in triplicate on the array. After hybridization, digital imaging systems are used to capture and quantify the relative fluorescence intensities of each of the hybridized fluorophores. The resulting ratio of the fluorescence intensities is proportional to the ratio of the copy numbers of DNA sequences in the two samples. In some embodiments, where there are chromosomal deletions or multiplications, differences in the ratio of the signals from the two labels are detected and the ratio provides a measure of the copy number. Array-based CGH can also be performed with single-color labeling. In single color CGH, a control (e.g., control nucleic acid sample, such as from a healthy cell/tissue) is labeled and hybridized to one array and absolute signals are read, and a test sample (e.g., a nucleic acid sample obtained from an individual or from a tumor, or a tissue or liquid biopsy) is labeled and hybridized to a second array (with identical content) and absolute signals are read. Copy number differences are calculated based on absolute signals from the two arrays.

In some embodiments, an ABL1 fusion nucleic acid molecule of the disclosure is detected using an amplification-based method. As is known in the art, in such amplification-based methods, a sample of nucleic acids, such as a sample obtained from an individual, a tumor or a tissue or liquid biopsy, is used as a template in an amplification reaction (e.g., Polymerase Chain Reaction (PCR)) using one or more oligonucleotides or primers, e.g., such as one or more oligonucleotides or primers provided herein. The presence of a fusion nucleic acid molecule of the disclosure in the sample can be determined based on the presence or absence of an amplification product. Quantitative amplification methods are also known in the art and may be used according to the methods provided herein. Methods of measurement of DNA copy number at microsatellite loci using quantitative PCR analysis are known in the art. The known nucleotide sequence for genes is sufficient to enable one of skill in the art to routinely select primers to amplify any portion of the gene. Fluorogenic quantitative PCR can also be used. In fluorogenic quantitative PCR, quantitation is based on the amount of fluorescence signals, e.g., TaqMan and Sybr green.

Other amplification methods suitable for use according to the methods provided herein include, e.g., ligase chain reaction (LCR), transcription amplification, self-sustained sequence replication, dot PCR, and linker adapter PCR.

In some embodiments, an ABL1 fusion nucleic acid molecule of the disclosure is detected using a sequencing method. Any method of sequencing known in the art can be used to detect an ABL1 fusion nucleic acid molecule provided herein. Exemplary sequencing methods that may be used to detect a fusion nucleic acid molecule provided herein include those based on techniques developed by Maxam and Gilbert or Sanger. Automated sequencing procedures may also be used, e.g., including sequencing by mass spectrometry.

In some embodiments, an ABL1 fusion nucleic acid molecule of the disclosure is detected using hybrid capture-based sequencing (hybrid capture-based NGS), e.g., using adaptor ligation-based libraries. See, e.g., Frampton, G. M. et al. (2013) Nat. Biotech. 31:1023-1031, which is hereby incorporated by reference. In some embodiments, an ABL1 fusion nucleic acid molecule of the disclosure is detected using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than 10⁵ molecules may be sequenced simultaneously). Next generation sequencing methods suitable for use according to the methods provided herein are known in the art and include, without limitation, massively parallel short-read sequencing, template-based sequencing, pyrosequencing, real-time sequencing comprising imaging the continuous incorporation of dye-labeling nucleotides during DNA synthesis, nanopore sequencing, sequencing by hybridization, nano-transistor array based sequencing, polony sequencing, scanning tunneling microscopy (STM)-based sequencing, or nanowire-molecule sensor based sequencing. See, e.g., Metzker, M. (2010) Nature Biotechnology Reviews 11:31-46, which is hereby incorporated by reference. Exemplary NGS methods and platforms that may be used to detect an ABL1 fusion nucleic acid molecule provided herein include, without limitation, the HeliScope Gene Sequencing system from Helicos BioSciences (Cambridge, MA., USA), the PacBio RS system from Pacific Biosciences (Menlo Park, CA, USA), massively parallel short-read sequencing such as the Solexa sequencer and other methods and platforms from Illumina Inc. (San Diego, CA, USA), 454 sequencing from 454 LifeSciences (Branford, CT, USA), Ion Torrent sequencing from ThermoFisher (Waltham, MA, USA), or the SOLID sequencer from Applied Biosystems (Foster City, CA, USA). Additional exemplary methods and platforms that may be used to detect a fusion nucleic acid molecule provided herein include, without limitation, the Genome Sequencer (GS) FLX System from Roche (Basel, CHE), the G.007 polonator system, the Solexa Genome Analyzer, HiSeq 2500, HiSeq3000, HiSeq 4000, and NovaSeq 6000 platforms from Illumina Inc. (San Diego, CA, USA).

In some embodiments of any of the methods provided herein, the methods may comprise one or more of the steps of: (i) obtaining a sample from an individual (e.g., an individual suspected of having or determined to have cancer), (ii) extracting nucleic acid molecules (e.g., a mixture of tumor or cancer nucleic acid molecules and non-tumor or non-cancer nucleic acid molecules) from the sample, (iii) ligating one or more adapters to the nucleic acid molecules extracted from the sample (e.g., one or more amplification primers, flow cell adaptor sequences, substrate adapter sequences, sample index sequences, or unique molecular identifier (UMI) sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules (e.g., capture nucleic acid molecules) that each comprise a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the individual (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in a graphical user interface of an online or web-based healthcare portal. In some instances, the report is transmitted via a computer network or peer-to-peer connection.

In some embodiments of any of the methods provided herein, the methods may comprise one or more of the steps of: (a) providing a plurality of nucleic acid molecules obtained from a sample from an individual (e.g., an individual suspected of having or determined to have cancer), wherein the plurality of nucleic acid molecules comprises nucleic acid molecules corresponding to an ABL1 fusion nucleic acid molecule of the disclosure; (b) ligating one or more adapters onto one or more nucleic acid molecules from the plurality of nucleic acid molecules; (c) amplifying the one or more ligated nucleic acid molecules from the plurality of nucleic acid molecules; (d) capturing amplified nucleic acid molecules from the amplified nucleic acid molecules; (e) sequencing, by a sequencer, the captured nucleic acid molecules to obtain a plurality of sequence reads that represent the captured nucleic acid molecules, wherein one or more of the plurality of sequence reads correspond to the ABL1 fusion nucleic acid molecule; (f) analyzing the plurality of sequence reads; and (g) based on the analysis, detecting the presence or absence of the ABL1 fusion nucleic acid molecule in the sample. In some embodiments, the methods further comprise receiving, at one or more processors, sequence read data for the plurality of sequence reads. In some embodiments, the analyzing the plurality of sequence reads comprises identifying, using the one or more processors, the presence or absence of sequence reads corresponding to the ABL1 fusion nucleic acid molecule. In some embodiments, the amplified nucleic acid molecules are captured by hybridization with one or more bait molecules.

In some embodiments of any of the methods provided herein, the methods may comprise one or more of the steps of: (a) providing a sample from an individual (e.g., an individual suspected of having or determined to have cancer), wherein the sample comprises a plurality of nucleic acid molecules; (b) preparing a nucleic acid sequencing library from the plurality of nucleic acid molecules in the sample; (c) amplifying said library; (d) selectively enriching for one or more nucleic acid molecules comprising nucleotide sequences corresponding to an ABL1 fusion nucleic acid molecule of the disclosure in said library to produce an enriched sample; (e) sequencing the enriched sample, thereby producing a plurality of sequence reads; (f) analyzing the plurality of sequence reads for the presence of the ABL1 fusion nucleic acid molecule; (g) detecting, based on the analyzing step, the presence or absence of the ABL1 fusion nucleic acid molecule in the sample from the individual.

In some embodiments of any of the methods provided herein, the plurality of nucleic acid molecules comprises a mixture of cancer nucleic acid molecules and non-cancer nucleic acid molecules. In some embodiments, the cancer nucleic acid molecules are derived from a tumor portion of a heterogeneous tissue biopsy sample, and the non-cancer nucleic acid molecules are derived from a normal portion of the heterogeneous tissue biopsy sample. In some embodiments, the sample comprises a liquid biopsy sample, and the cancer nucleic acid molecules are derived from a circulating tumor DNA (ctDNA) fraction of the liquid biopsy sample; and the non-cancer nucleic acid molecules are derived from a non-tumor fraction of the liquid biopsy sample or a cell-free DNA (cfDNA) fraction of the liquid biopsy sample.

In some embodiments of any of the methods, the one or more adapters comprise amplification primers, flow cell adaptor sequences, substrate adapter sequences, sample index sequences, or unique molecular identifier (UMI) sequences. In some embodiments, the one or more adapters comprise one or more sample index sequences. As is known in the art, sample indexes allow the sequencing of multiple samples on the same instrument flow cell or chip (i.e., multiplexing). Sample indexes are typically between about 8 and about 10 bases in length, and comprise a nucleotide sequence specific to a sample that is used to assign sequence reads to the correct sample during data analysis. In some embodiments, the one or more adapters comprise one or more unique molecule identifiers (UMIs). As is known in the art, UMIs comprise short nucleotide sequences that include a unique barcode that is incorporated into each molecule in a given sample library. UMIs are useful for identifying PCR duplicates created during library amplification steps, and/or for reducing the rate of false-positive variant calls and increasing variant detection, since variant alleles present in the original sample (true variants) can be distinguished from errors introduced during library preparation, target enrichment, or sequencing.

In some embodiments, the selectively enriching comprises: (a) combining one or more bait molecules with the library, thereby hybridizing the one or more bait molecules to one or more nucleic acid molecules comprising nucleotide sequences corresponding to the ABL1 fusion nucleic acid molecule and producing nucleic acid hybrids; and (b) isolating the nucleic acid hybrids to produce the enriched sample. In some embodiments, the captured nucleic acid molecules are captured from the amplified nucleic acid molecules by hybridization to one or more bait molecules. In some embodiments, the amplifying comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencer comprises a next generation sequencer.

In some embodiments of any of the methods provided herein, the methods further comprise selectively enriching for one or more nucleic acids in the sample comprising nucleotide sequences corresponding to an ABL1 fusion nucleic acid molecule of the disclosure. In some embodiments, the selectively enriching produces an enriched sample. In some embodiments, the selectively enriching comprises: (a) combining one or more bait molecules with the sample, thereby hybridizing the one or more bait molecules to one or more nucleic acids in the sample comprising nucleotide sequences corresponding to the ABL1 fusion nucleic acid molecule and producing nucleic acid hybrids; and (b) isolating the nucleic acid hybrids to produce the enriched sample. In some embodiments, the selectively enriching comprises amplifying the one or more nucleic acids comprising nucleotide sequences corresponding to the ABL1 fusion nucleic acid molecule using a polymerase chain reaction (PCR) to produce an enriched sample. In some embodiments, the methods further comprise sequencing the enriched sample.

In some embodiments of any of the methods provided herein, the methods further comprise analyzing sequence data (e.g., obtained from sequencing as described above), for the presence or absence of one or more alterations (e.g., a base substitution, a short insertion/deletion (indel), a copy number alteration, or a genomic rearrangement) in one or more genes (e.g., one or more cancer-related genes, or a panel of known/suspected oncogenes and/or tumor suppressors, or any combination thereof). In some embodiments, the presence or absence of one or more gene alterations of the disclosure is detected using any suitable method known in the art, e.g., as described in Frampton et al., (2013) Nat Biotechnol, 31:1023-1031. In some embodiments, base substitution alterations are detected using Bayesian methodology, which allows detection of novel somatic mutations at low mutant allele frequency (MAF) and increased sensitivity for mutations at hotspot sites through the incorporation of tissue-specific prior expectations. See, e.g., Kim et al., Cancer Discov (2011) 1:44-53 and Frampton et al., (2013) Nat Biotechnol, 31:1023-1031. In some embodiments, insertion/deletion (indel) alterations are detected using any suitable method, such as de novo local assembly, e.g., using the de Bruijn approach, see, e.g., Compeau et al., Nat Biotechnol (2011) 29:987-991 and Frampton et al., (2013) Nat Biotechnol, 31:1023-1031. In some embodiments, gene fusion and genomic rearrangement alterations are detected using any suitable method, such as by analyzing chimeric read pairs (read pairs for which reads map to separate chromosomes, or at a distance of over 10 Mbp), see, e.g., Frampton et al., (2013) Nat Biotechnol, 31:1023-1031. In some embodiments, rearrangements are annotated for predicted function (e.g., creation of fusion gene or tumor suppressor inactivation). In some embodiments, the one or more genes comprise the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKNIA, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKARIA, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCA1P, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene, or any combination thereof. In some embodiments, the one or more genes comprise the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene, or any combination thereof.

In some embodiments of any of the methods provided herein, the methods further comprise generating a molecular profile for the individual or the sample, based, at least in part, on detecting the presence or absence of an ABL1 fusion nucleic acid molecule of the disclosure. In some embodiments, the molecular profile for the individual or sample further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the molecular profile further comprises results from a nucleic acid sequencing-based test. In some instances, a molecular profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some embodiments of any of the methods provided herein, the methods further comprise selecting a treatment, administering a treatment, or applying a treatment to the individual based on the generated molecular profile, wherein the treatment comprises an anti-cancer therapy, e.g., as described herein, e.g., an ABL1-targeted therapy. In some embodiments of any of the methods provided herein, the methods further comprise generating a report indicating the presence or absence of an ABL1 fusion nucleic acid molecule of the disclosure, in the sample. In some embodiments of any of the methods provided herein, the methods further comprise generating, by one or more processors, a report indicating the presence or absence of an ABL1 fusion nucleic acid molecule of the disclosure in the sample. In some embodiments, the report comprises the generated molecular profile. In some embodiments, the methods further comprise providing or transmitting the report, e.g., as described below. In some embodiments, the report is transmitted via a computer network or a peer-to-peer connection. In some instances, all or a portion of the report may be displayed in a graphical user interface of an online or web-based healthcare portal.

In some embodiments of any of the methods provided herein, the methods for determining the presence or absence of an ABL1 fusion nucleic acid molecule, may be implemented as part of a genomic profiling process that comprises identification of the presence of variant sequences at one or more gene loci (e.g., one or more gene loci as listed above) in a sample derived from an individual as part of detecting, monitoring, predicting a risk factor, or selecting a treatment for a particular disease, e.g., cancer. In some instances, the variant panel selected for genomic profiling may comprise the detection of variant sequences at a selected set of gene loci (e.g., one or more gene loci as listed above). In some instances, the variant panel selected for genomic profiling may comprise detection of variant sequences at a number of gene loci (e.g., one or more gene loci as listed above) through comprehensive genomic profiling (CGP), a next-generation sequencing (NGS) approach used to assess hundreds of genes (including relevant cancer biomarkers) in a single assay. Inclusion of the disclosed methods for determining the presence or absence of an ABL1 fusion nucleic acid molecule as part of a genomic profiling process can improve the validity of, e.g., disease detection calls by, for example, independently confirming the presence of the ABL1 fusion nucleic acid molecule in a given patient sample.

The disclosed methods may be used with any of a variety of samples, e.g., as described in further detail below. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor or cancer nucleic acid molecules and non-tumor or non-cancer nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor or cancer nucleic acid molecules may be derived from a circulating tumor DNA (ctDNA) fraction of the liquid biopsy sample while the non-tumor or non-cancer nucleic acid molecules may be derived from a non-tumor or non-cancer, cell-free DNA (cfDNA) fraction of the liquid biopsy sample.

In some embodiments of any of the methods provided herein, the method further comprises determining the circulating tumor DNA (ctDNA) fraction of a liquid biopsy sample.

### (ii) Detection of ABL1 Fusion Polypeptides

Also provided herein are methods of detecting an ABL1 fusion polypeptide of the disclosure, or a fragment thereof, e.g., an ABL1 fusion polypeptide encoded by an ABL1 fusion nucleic acid molecule of the disclosure, or a fragment thereof.

An ABL1 fusion polypeptide provided herein, or a fragment thereof, may be detected or measured, e.g., in a sample obtained from an individual, using any method known in the art, such as using antibodies (e.g., an antibody described herein), mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography).

In some embodiments, an ABL1 fusion polypeptide provided herein, or a fragment thereof, can be distinguished from a reference polypeptide, e.g., a non-mutant or wild type protein or polypeptide, with an antibody or antibody fragment that reacts differentially with a mutant protein or polypeptide (e.g., an ABL1 fusion polypeptide provided herein or a fragment thereof) as compared to a reference protein or polypeptide. In some embodiments, an ABL1 fusion polypeptide of the disclosure, or a fragment thereof, can be distinguished from a reference polypeptide, e.g., a non-mutant or wild type protein or polypeptide, by reaction with a detection reagent, e.g., a substrate, e.g., a substrate for catalytic activity, e.g., phosphorylation.

In some aspects, methods of detection of an ABL1 fusion polypeptide of the disclosure (e.g., an ABL1 fusion polypeptide encoded by an ABL1 fusion nucleic acid molecule of the disclosure), or a fragment thereof, are provided, comprising contacting a sample, e.g., a sample described herein, comprising an ABL1 fusion polypeptide described herein, with a detection reagent provided herein (e.g., an antibody of the disclosure), and determining if the ABL1 fusion polypeptide is present in the sample.

(iii) Detection Reagents

In some aspects, provided herein are reagents for detecting an ABL1 fusion nucleic acid molecule of the disclosure, or a fragment thereof, e.g., according to the methods of detection provided herein. In some embodiments, a detection reagent provided herein comprises a nucleic acid molecule, e.g., a DNA, RNA, or mixed DNA/RNA molecule, comprising a nucleotide sequence that is complementary to a nucleotide sequence on a target nucleic acid molecule, e.g., a nucleic acid molecule that is or comprises an ABL1 fusion nucleic acid molecule described herein or a fragment or portion thereof.

In other aspects, provided herein are reagents for detecting an ABL1 fusion polypeptide of the disclosure, or a fragment thereof, e.g., an ABL1 fusion polypeptide encoded by an ABL1 fusion nucleic acid molecule of the disclosure, or a fragment thereof, e.g., according to the methods of detection provided herein. In some embodiments, a detection reagent provided herein comprises an antibody or antibody fragment that specifically binds to an ABL1 fusion polypeptide of the disclosure, or to a fragment thereof.

### Baits

In some embodiments, nucleic acids corresponding to a gene involved in an ABL1 fusion nucleic acid molecule described herein, e.g., an ABL1 gene, and/or a corresponding gene fusion partner as described herein (e.g., e.g., any of BMP2K, EHMT1, MED27, RFFL, SLC27A4, BCR, NUP214, AIF1L, RCSD1, APOOL, DNAH2, RHEX/C1orf186, RALGPS1, TSC1, TSPAN18, ZNF804B, EXOSC2, or SNX2), are captured (e.g., from amplified nucleic acids) by hybridization with a bait molecule. Provided herein are bait molecules suitable for the detection of an ABL1 fusion nucleic acid molecule of the disclosure.

In some embodiments, a bait molecule comprises a capture nucleic acid molecule configured to hybridize to a target nucleic acid molecule comprising an ABL1 fusion nucleic acid molecule of the disclosure, or a fragment or portion thereof. In some embodiments, the capture nucleic acid molecule is configured to hybridize to the ABL1 fusion nucleic acid molecule of the target nucleic acid molecule.

In some embodiments, the capture nucleic acid molecule is configured to hybridize to a fragment of an ABL1 fusion nucleic acid molecule of the disclosure. In some embodiments, the fragment comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the fragment comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the fragment comprises a breakpoint or fusion junction of an ABL1 fusion nucleic acid molecule of the disclosure.

In some embodiments, the capture nucleic acid molecule comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the capture nucleic acid molecule comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length.

In some embodiments, the capture nucleic acid molecule is configured to hybridize to a breakpoint of an ABL1 fusion nucleic acid molecule of the disclosure, and may further hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the breakpoint.

In some embodiments, the capture nucleic acid molecule is configured to hybridize to a nucleotide sequence in an intron or an exon of an ABL1 gene, or in a breakpoint joining the introns or exons of an ABL1 gene (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides) to an intron or exon of another gene, such as a corresponding gene fusion partner as described herein (e.g., any of BMP2K, EHMT1, MED27, RFFL, SLC27A4, BCR, NUP214, AIF1L, RCSD1, APOOL, DNAH2, RHEX/C1orf186, RALGPS1, TSC1, TSPAN18, ZNF804B, EXOSC2, or SNX2).

In some embodiments, the capture nucleic acid molecule is a DNA, RNA, or a DNA/RNA molecule. In some embodiments, the capture nucleic acid molecule comprises any of between about 50 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the capture nucleic acid molecule comprises any of between about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides. In some embodiments, the capture nucleic acid molecule comprises about 150 nucleotides. In some embodiments, the capture nucleic acid molecule is about 150 nucleotides. In some embodiments, the capture nucleic acid molecule comprises about 170 nucleotides. In some embodiments, the capture nucleic acid molecule is about 170 nucleotides.

In some embodiments, a bait provided herein comprises a DNA, RNA, or a DNA/RNA molecule. In some embodiments, a bait provided herein includes a label, a tag or detection reagent. In some embodiments, the label, tag or detection reagent is a radiolabel, a fluorescent label, an enzymatic label, a sequence tag, biotin, or another ligand. In some embodiments, a bait provided herein includes a detection reagent such as a fluorescent marker. In some embodiments, a bait provided herein includes (e.g., is conjugated to) an affinity tag or reagent, e.g., that allows capture and isolation of a hybrid formed by a bait and a nucleic acid molecule hybridized to the bait. In some embodiments, the affinity tag or reagent is an antibody, an antibody fragment, biotin, or any other suitable affinity tag or reagent known in the art. In some embodiments, a bait is suitable for solution phase hybridization.

Baits can be produced and used according to methods known in the art, e.g., as described in WO2012092426A1 and/or or in Frampton et al (2013) Nat Biotechnol, 31:1023-1031, incorporated herein by reference. For example, biotinylated baits (e.g., RNA baits) can be produced by obtaining a pool of synthetic long oligonucleotides, originally synthesized on a microarray, and amplifying the oligonucleotides to produce the bait sequences. In some embodiments, the baits are produced by adding an RNA polymerase promoter sequence at one end of the bait sequences, and synthesizing RNA sequences using RNA polymerase. In one embodiment, libraries of synthetic oligodeoxynucleotides can be obtained from commercial suppliers, such as Agilent Technologies, Inc., and amplified using known nucleic acid amplification methods.

In some embodiments, a bait provided herein is between about 100 nucleotides and about 300 nucleotides. In some embodiments, a bait provided herein is between about 130 nucleotides and about 230 nucleotides. In some embodiments, a bait provided herein is between about 150 nucleotides and about 200 nucleotides. In some embodiments, a bait provided herein comprises a target-specific bait sequence (e.g., a capture nucleic acid molecule described herein) and universal tails on each end. In some embodiments, the target-specific sequence, e.g., a capture nucleic acid molecule described herein, is between about 40 nucleotides and about 300 nucleotides. In some embodiments, the target-specific sequence, e.g., a capture nucleic acid molecule described herein, is between about 100 nucleotides and about 200 nucleotides. In some embodiments, the target-specific sequence, e.g., a capture nucleic acid molecule described herein, is between about 120 nucleotides and about 170 nucleotides. In some embodiments, the target-specific sequence, e.g., a capture nucleic acid molecule described herein, is about 150 nucleotides or about 170 nucleotides. In some embodiments, a bait provided herein comprises an oligonucleotide comprising about 200 nucleotides, of which about 150 nucleotides or about 170 nucleotides are target-specific (e.g., a capture nucleic acid molecule described herein), and the other 50 nucleotides or 30 nucleotides (e.g., 25 or 15 nucleotides on each end of the bait) are universal arbitrary tails, e.g., suitable for PCR amplification.

In some embodiments, a bait provided herein hybridizes to a nucleotide sequence corresponding to an intron or an exon of one gene of an ABL1 fusion molecule described herein (e.g., an ABL1 gene), in an intron or an exon of the other gene of a fusion molecule described herein (e.g., a corresponding gene fusion partner as described herein, e.g., any of BMP2K, EHMT1, MED27, RFFL, SLC27A4, BCR, NUP214, AIF1L, RCSD1, APOOL, DNAH2, RHEX/C1orf186, RALGPS1, TSC1, TSPAN18, ZNF804B, EXOSC2, or SNX2), and/or a breakpoint joining the introns and/or exons.

The baits described herein can be used for selection of exons and short target sequences.

In some embodiments, a bait of the disclosure distinguishes a nucleic acid molecule, e.g., a genomic or transcribed nucleic acid molecule, e.g., a cDNA or RNA, having a breakpoint of an ABL1 fusion nucleic acid molecule described herein, from a reference nucleotide sequence, e.g., a nucleotide sequence not having the breakpoint.

In some embodiments, the bait hybridizes to a breakpoint of an ABL1 fusion nucleic acid molecule described herein, and to a sequence on either side of the breakpoint (e.g., any of 1, 2, 3, 4, 5, 6, 7, 8, 9, or 10 nucleotides on either side of the breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the breakpoint).

### Probes

Also provided herein are probes, e.g., nucleic acid molecules, suitable for the detection of an ABL1 fusion nucleic acid molecule of the disclosure. In some embodiments, a probe provided herein comprises a nucleic acid sequence configured to hybridize to a target nucleic acid molecule that is or comprises an ABL1 fusion nucleic acid molecule of the disclosure, or a fragment or portion thereof. In some embodiments, the probe comprises a nucleic acid sequence configured to hybridize to the ABL1 fusion nucleic acid molecule of the disclosure, or the fragment or portion thereof, of the target nucleic acid molecule. In some embodiments, the probe comprises a nucleic acid sequence configured to hybridize to a fragment or portion of the ABL1 fusion nucleic acid molecule of the target nucleic acid molecule. In some embodiments, the fragment or portion comprises between about 5 and about 25 nucleotides, between about 5 and about 300 nucleotides, between about 100 and about 300 nucleotides, between about 130 and about 230 nucleotides, or between about 150 and about 200 nucleotides.

In some embodiments, the probe comprises a nucleotide sequence configured to hybridize to a breakpoint of an ABL1 fusion nucleic acid molecule of the disclosure, and may be further configured to hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the breakpoint.

In some embodiments, the probe comprises a nucleotide sequence configured to hybridize to a nucleotide sequence in an intron or an exon of a gene involved in an ABL1 fusion nucleic acid molecule described herein, e.g., an ABL1 gene, or in a breakpoint joining the introns or exons of the gene (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides) to an intron or exon of another gene (e.g., a corresponding gene fusion partner as described herein, e.g., any of BMP2K, EHMT1, MED27, RFFL, SLC27A4, BCR, NUP214, AIF1L, RCSD1, APOOL, DNAH2, RHEX/C1orf186, RALGPS1, TSC1, TSPAN18, ZNF804B, EXOSC2, or SNX2).

In some embodiments, the probe comprises a nucleic acid molecule which is a DNA, RNA, or a DNA/RNA molecule. In some embodiments, the probe comprises a nucleic acid molecule comprising any of between about 10 and about 20 nucleotides, between about 12 and about 20 nucleotides, between about 10 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the probe comprises a nucleic acid molecule comprising any of 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides. In some embodiments, the probe comprises a nucleic acid molecule comprising any of between about 40 nucleotides and about 50 nucleotides, about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the probe comprises a nucleic acid molecule comprising between about 12 and about 20 nucleotides.

In some embodiments, a probe provided herein comprises a DNA, RNA, or a DNA/RNA molecule. In some embodiments, a probe provided herein includes a label or a tag. In some embodiments, the label or tag is a radiolabel (e.g., a radioisotope), a fluorescent label (e.g., a fluorescent compound), an enzymatic label, an enzyme co-factor, a sequence tag, biotin, or another ligand. In some embodiments, a probe provided herein includes a detection reagent such as a fluorescent marker. In some embodiments, a probe provided herein includes (e.g., is conjugated to) an affinity tag, e.g., that allows capture and isolation of a hybrid formed by a probe and a nucleic acid molecule hybridized to the probe. In some embodiments, the affinity tag is an antibody, an antibody fragment, biotin, or any other suitable affinity tag or reagent known in the art. In some embodiments, a probe is suitable for solution phase hybridization.

In some embodiments, probes provided herein may be used according to the methods of detection of ABL1 fusion nucleic acid molecules provided herein. For example, a probe provided herein may be used for detecting an ABL1 fusion nucleic acid molecule of the disclosure in a sample, e.g., a sample obtained from an individual. In some embodiments, the probe may be used for identifying cells or tissues that express an ABL1 fusion nucleic acid molecule of the disclosure, e.g., by measuring levels of the ABL1 fusion nucleic acid molecule. In some embodiments, the probe may be used for detecting levels of an ABL1 fusion nucleic acid molecule of the disclosure, e.g., mRNA levels, in a sample of cells from an individual.

In some embodiments, a probe provided herein specifically hybridizes to a nucleic acid molecule comprising a rearrangement (e.g., a deletion, inversion, insertion, duplication, or other rearrangement) resulting in an ABL1 fusion nucleic acid molecule of the disclosure.

In some embodiments, a probe of the disclosure distinguishes a nucleic acid, e.g., a genomic or transcribed nucleic acid, e.g., a cDNA or RNA, having a breakpoint of an ABL1 fusion nucleic acid molecule of the disclosure from a reference nucleotide sequence, e.g., a nucleotide sequence not having the breakpoint.

Also provided herein are isolated pairs of allele-specific probes, wherein, for example, the first probe of the pair specifically hybridizes to an ABL1 fusion nucleic acid molecule of the disclosure, and the second probe of the pair specifically hybridizes to a corresponding wild type sequence (e.g., a wild type ABL1 nucleic acid molecule; and/or a wild type nucleic acid molecule corresponding to a gene fusion partner as described herein, e.g., any of BMP2K, EHMT1, MED27, RFFL, SLC27A4, BCR, NUP214, AIF1L, RCSD1, APOOL, DNAH2, RHEX/C1orf186, RALGPS1, TSC1, TSPAN18, ZNF804B, EXOSC2, or SNX2). Probe pairs can be designed and produced for any of the ABL1 fusion nucleic acid molecules described herein and are useful in detecting a somatic mutation in a sample. In some embodiments, a first probe of a pair specifically hybridizes to a mutation (e.g., the breakpoint of an alteration, rearrangement, inversion, duplication, deletion, insertion or translocation resulting in an ABL1 fusion nucleic acid molecule described herein), and a second probe of a pair specifically hybridizes to a sequence upstream or downstream of the mutation.

In some embodiments, one or more probes provided herein are suitable for use in in situ hybridization methods, e.g., as described above, such as FISH.

Chromosomal probes, e.g., for use in the FISH methods described herein, are typically about 50 to about 10⁵ nucleotides in length. Longer probes typically comprise smaller fragments of about 100 to about 500 nucleotides. Probes that hybridize with centromeric DNA and locus-specific DNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, chromosome (e.g., human chromosome) along with the normal chromosome complement of the host, and chromosomes purified by flow cytometry or microdissection. The region of interest can be isolated through cloning, or by site-specific amplification via the polymerase chain reaction (PCR). Probes of the disclosure may also hybridize to RNA molecules, e.g., mRNA, such as an RNA that is or comprises an ABL1 fusion nucleic acid molecule of the disclosure.

In some embodiments, probes, such as probes for use in the FISH methods described herein, are used for determining whether a cytogenetic abnormality is present in one or more cells, e.g., in a region of a chromosome or an RNA bound by one or more probes provided herein. The cytogenetic abnormality may be a cytogenetic abnormality that results in an ABL1 fusion nucleic acid molecule of the disclosure. Examples of such cytogenetic abnormalities include, without limitation, deletions (e.g., deletions of entire chromosomes or deletions of fragments of one or more chromosomes), duplications (e.g., of entire chromosomes, or of regions smaller than an entire chromosome), translocations (e.g., non-reciprocal translocations, balanced translocations, reciprocal translocations), intra-chromosomal inversions, point mutations, deletions, gene copy number changes, germ-line mutations, and gene expression level changes.

In some embodiments, probes, such as probes for use in the FISH methods described herein, are labeled such that a chromosomal region or a region on an RNA to which the probes hybridize can be detected. Probes typically are directly labeled with a fluorophore, allowing the probe to be visualized without a secondary detection molecule. Probes can also be labeled by nick translation, random primer labeling or PCR labeling. Labeling may be accomplished using fluorescent (direct)- or haptene (indirect)-labeled nucleotides. Representative, non-limiting examples of labels include: AMCA-6-dUTP, CascadeBlue-4-dUTP, Fluorescein-12-dUTP, Rhodamine-6-dUTP, TexasRed-6-dUTP, Cy3-6-dUTP, Cy5-dUTP, Biotin (BIO)-11-dUTP, Digoxygenin (DIG)-11-dUTP and Dinitrophenyl (DNP)-11-dUTP. Probes can also be indirectly labeled with biotin or digoxygenin, or labeled with radioactive isotopes such as 32P and 3H, and secondary detection molecules may be used, or further processing may be performed, to visualize the probes. For example, a probe labeled with biotin can be detected by avidin conjugated to a detectable marker, e.g., avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase. Probes can also be prepared such that a fluorescent or other label is added after hybridization of the probe to its target to detect that the probe hybridized to the target. For example, probes can be used that have antigenic molecules incorporated into the nucleotide sequence. After hybridization, these antigenic molecules are detected, for example, using specific antibodies reactive with the antigenic molecules. Such antibodies can, for example, themselves incorporate a fluorochrome, or can be detected using a second antibody with a bound fluorochrome. For fluorescent probes, e.g., used in FISH techniques, fluorescence can be viewed with a fluorescence microscope equipped with an appropriate filter for each fluorophore, or by using dual or triple band-pass filter sets to observe multiple fluorophores. Alternatively, techniques such as flow cytometry can be used to examine the hybridization pattern of the chromosomal probes.

In some embodiments, the probe hybridizes to a breakpoint of an ABL1 fusion nucleic acid molecule of the disclosure and a sequence on either side of the breakpoint (e.g., any of 1, 2, 3, 4, 5, 6, 7, 8, 9, or 10 nucleotides on either side of the breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the breakpoint).

### Oligonucleotides

In some aspects, provided herein are oligonucleotides, e.g., useful as primers. In some embodiments, an oligonucleotide, e.g., a primer, provided herein comprises a nucleotide sequence configured to hybridize to a target nucleic acid molecule that is or comprises an ABL1 fusion nucleic acid molecule of the disclosure, or a fragment or portion thereof. In some embodiments, the oligonucleotide comprises a nucleotide sequence configured to hybridize to the ABL1 fusion nucleic acid molecule of the target nucleic acid molecule. In some embodiments, the oligonucleotide comprises a nucleotide sequence configured to hybridize to a fragment or portion of the ABL1 fusion nucleic acid molecule of the target nucleic acid molecule.

In some embodiments, the oligonucleotide, e.g., the primer, comprises a nucleotide sequence configured to hybridize to a breakpoint of an ABL1 fusion nucleic acid molecule of the disclosure, and may be further configured to hybridize to between about 10 and about 12, about 12 and about 15, about 15 and about 17, about 17 and about 20, about 20 and about 25, or about 25 and about 30, or more nucleotides flanking either side of the breakpoint.

In some embodiments, the oligonucleotide, e.g., the primer, comprises a nucleotide sequence configured to hybridize to a nucleotide sequence in an intron or an exon of a gene involved in an ABL1 fusion nucleic acid mole of the disclosure (e.g., an ABL1 gene), to a breakpoint of an ABL1 fusion nucleic acid molecule described herein, and/or to an intron or exon of another gene (e.g., a corresponding gene fusion partner as described herein, e.g., any of BMP2K, EHMT1, MED27, RFFL, SLC27A4, BCR, NUP214, AIF1L, RCSD1, APOOL, DNAH2, RHEX/C1orf186, RALGPS1, TSC1, TSPAN18, ZNF804B, EXOSC2, or SNX2).

In some embodiments, the oligonucleotide comprises a nucleotide sequence corresponding to an ABL1 fusion nucleic acid molecule of the disclosure. In some embodiments, the oligonucleotide comprises a nucleotide sequence corresponding to a fragment or a portion of the ABL1 fusion nucleic acid molecule. In some embodiments, the fragment or portion comprises between about 10 and about 30 nucleotides, between about 12 and about 20 nucleotides, or between about 12 and about 17 nucleotides. In some embodiments, the oligonucleotide comprises a nucleotide sequence complementary to an ABL1 fusion nucleic acid molecule provided herein. In some embodiments, the oligonucleotide comprises a nucleotide sequence complementary to a fragment or a portion of the ABL1 fusion nucleic acid molecule provided herein. In some embodiments, the fragment or portion comprises between about 10 and about 30 nucleotides, between about 12 and about 20 nucleotides, or between about 12 and about 17 nucleotides.

In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence, e.g., under high stringency conditions. In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence under conditions that allow a polymerization reaction (e.g., PCR) to occur.

In some embodiments, an oligonucleotide, e.g., a primer, provided herein may be useful for initiating DNA synthesis via PCR (polymerase chain reaction) or a sequencing method. In some embodiments, the oligonucleotide may be used to amplify a nucleic acid molecule that is or comprises an ABL1 fusion nucleic acid molecule of the disclosure, or a fragment thereof, e.g., using PCR. In some embodiments, the oligonucleotide may be used to sequence a nucleic acid molecule that is or comprises an ABL1 fusion nucleic acid molecule provided herein, or a fragment thereof. In some embodiments, the oligonucleotide may be used to amplify a nucleic acid molecule comprising a breakpoint of an ABL1 fusion nucleic acid molecule described herein, e.g., using PCR. In some embodiments, the oligonucleotide may be used to sequence a nucleic acid molecule comprising a breakpoint of an ABL1 fusion nucleic acid molecule described herein.

In some embodiments, pairs of oligonucleotides, e.g., pairs of primers, are provided herein, which are configured to hybridize to a nucleic acid molecule that is or comprises an ABL1 fusion nucleic acid molecule of the disclosure, or a fragment thereof. In some embodiments, a pair of oligonucleotides of the disclosure may be used for directing amplification of the ABL1 fusion nucleic acid molecule or fragment thereof, e.g., using a PCR reaction. In some embodiments, pairs of oligonucleotides, e.g., pairs of primers, are provided herein, which are configured to hybridize to a nucleic acid molecule comprising a breakpoint of an ABL1 fusion nucleic acid molecule described herein, e.g., for use in directing amplification of the corresponding ABL1 fusion nucleic acid molecule or fragment thereof, e.g., using a PCR reaction.

In some embodiments, an oligonucleotide, e.g., a primer, provided herein is a single stranded nucleic acid molecule, e.g., for use in sequencing or amplification methods. In some embodiments, an oligonucleotide provided herein is a double stranded nucleic acid molecule. In some embodiments, a double stranded oligonucleotide is treated, e.g., denatured, to separate its two strands prior to use, e.g., in sequencing or amplification methods. Oligonucleotides provided herein comprise a nucleotide sequence of sufficient length to hybridize to their target, e.g., an ABL1 fusion nucleic acid molecule of the disclosure, or a fragment thereof, and to prime the synthesis of extension products, e.g., during PCR or sequencing.

In some embodiments, an oligonucleotide, e.g., a primer, provided herein comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more deoxyribonucleotides or ribonucleotides. In some embodiments, an oligonucleotide provided herein comprises at least about 8 deoxyribonucleotides or ribonucleotides. In some embodiments, an oligonucleotide provided herein comprises at least about 10 deoxyribonucleotides or ribonucleotides. In some embodiments, an oligonucleotide provided herein comprises at least about 12 deoxyribonucleotides or ribonucleotides. In some embodiments, an oligonucleotide provided herein comprises at least about 15 deoxyribonucleotides or ribonucleotides. In some embodiments, an oligonucleotide provided herein comprises at least about 20 deoxyribonucleotides or ribonucleotides. In some embodiments, an oligonucleotide provided herein comprises at least about 30 deoxyribonucleotides or ribonucleotides. In some embodiments, an oligonucleotide provided herein comprises between about 10 and about 30 deoxyribonucleotides or ribonucleotides. In some embodiments, an oligonucleotide provided herein comprises between about 10 and about 25 deoxyribonucleotides or ribonucleotides. In some embodiments, an oligonucleotide provided herein comprises between about 10 and about 20 deoxyribonucleotides or ribonucleotides. In some embodiments, an oligonucleotide provided herein comprises between about 10 and about 15 deoxyribonucleotides or ribonucleotides. In some embodiments, an oligonucleotide provided herein comprises between about 12 and about 20 deoxyribonucleotides or ribonucleotides. In some embodiments, an oligonucleotide provided herein comprises between about 17 and about 20 deoxyribonucleotides or ribonucleotides. In some embodiments, the length and nucleotide sequence of an oligonucleotide provided herein is determined according to methods known in the art, e.g., based on factors such as the specific application (e.g., PCR, sequencing library preparation, sequencing), reaction conditions (e.g., buffers, temperature), and the nucleotide composition of the nucleotide sequence of the oligonucleotide or of its target complementary sequence.

In some embodiments, an oligonucleotide, e.g., a primer, of the disclosure distinguishes a nucleic acid, e.g., a genomic or transcribed nucleic acid, e.g., a cDNA or RNA, having a breakpoint of an ABL1 fusion nucleic acid molecule described herein from a reference nucleotide sequence, e.g., a nucleotide sequence not having the breakpoint.

In one aspect, provided herein is a primer or primer set for amplifying a nucleic acid molecule comprising a cytogenetic abnormality such as an alteration, rearrangement, chromosomal inversion, deletion, translocation, duplication, or other rearrangement resulting in an ABL1 fusion nucleic acid molecule of the disclosure. In another aspect, provided herein is a primer or primer set for amplifying a nucleic acid molecule comprising an alteration, rearrangement, chromosomal inversion, insertion, deletion, translocation, duplication or other rearrangement resulting in an ABL1 fusion nucleic acid molecule of the disclosure. In certain aspects, provided herein are allele-specific oligonucleotides, e.g., primers, wherein a first oligonucleotide of a pair specifically hybridizes to a mutation (e.g., a breakpoint of an ABL1 fusion nucleic acid molecule described herein), and a second oligonucleotide of a pair specifically hybridizes to a sequence upstream or downstream of the mutation. In certain aspects, provided herein are pairs of oligonucleotides, e.g., primers, wherein a first oligonucleotide of a pair specifically hybridizes to a sequence upstream of a mutation (e.g., a breakpoint of an ABL1 fusion nucleic acid molecule described herein), and a second oligonucleotide of the pair specifically hybridizes to a sequence downstream of the mutation.

In some embodiments, the oligonucleotide, e.g., the primer, hybridizes to a breakpoint of an ABL1 fusion nucleic acid molecule described herein and a sequence on either side of the breakpoint (e.g., any of 1, 2, 3, 4, 5, 6, 7, 8, 9, or 10 nucleotides on either side of the breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the breakpoint).

### Antibodies

Provided herein are antibodies or antibody fragments that specifically bind to an ABL1 fusion polypeptide of the disclosure, or a fragment thereof, e.g., an ABL1 fusion polypeptide encoded by an ABL1 fusion nucleic acid molecule of the disclosure, or a fragment thereof.

The antibody or antibody fragment may be of any suitable type of antibody or antibody fragment, including, but not limited to, a monoclonal, polyclonal, or multi-specific (e.g., a bispecific) antibody or antibody fragment, so long as the antibody or antibody fragment exhibits a specific antigen binding activity, e.g., binding to an ABL1 fusion polypeptide of the disclosure, or a fragment thereof.

In some embodiments, an ABL1 fusion polypeptide of the disclosure, or a fragment thereof, is used as an immunogen to generate one or more antibodies or antibody fragments of the disclosure, e.g., using standard techniques for polyclonal and monoclonal antibody preparation. In some embodiments, an ABL1 fusion polypeptide provided herein, is used to provide antigenic peptide fragments (e.g., comprising any of at least about 8, at least about 10, at least about 15, at least about 20, at least about 30 or more amino acids) for use as immunogens to generate one or more antibodies or antibody fragments of the disclosure, e.g., using standard techniques for polyclonal and monoclonal antibody preparation. As is known in the art, an antibody or antibody fragment of the disclosure may be prepared by immunizing a suitable (i.e., immunocompetent) subject such as a rabbit, goat, mouse, or other mammal or vertebrate. An appropriate immunogenic preparation can contain, for example, recombinantly-expressed or chemically-synthesized polypeptides, e.g., an ABL1 fusion polypeptide of the disclosure, or a fragment thereof. The preparation can further include an adjuvant, such as Freund's complete or incomplete adjuvant, or a similar immunostimulatory agent.

In some embodiments, an antibody or antibody fragment provided herein is a polyclonal antibody. Methods of producing polyclonal antibodies and fragments thereof are known in the art. In some embodiments, an antibody or antibody fragment provided herein is a monoclonal antibody, wherein a population of the antibody or fragment molecules contain only one species of an antigen binding site capable of immunoreacting or binding with a particular epitope, e.g., an epitope on an ABL1 fusion polypeptide provided herein. Methods of preparation of monoclonal antibodies and fragments thereof are known in the art, e.g., using standard hybridoma techniques originally described by Kohler and Milstein (1975) Nature 256:495-497, human B cell hybridoma techniques (see Kozbor et al., 1983, Immunol. Today 4:72), EBV-hybridoma techniques (see Cole et al., pp. 77-96 In Monoclonal Antibodies and Cancer Therapy, Alan R. Liss, Inc., 1985), or trioma techniques. The technology for producing hybridomas is well known (see generally Current Protocols in Immunology, Coligan et al. ed., John Wiley & Sons, New York, 1994). A monoclonal antibody or antibody fragment of the disclosure may also be identified and isolated by screening a recombinant combinatorial immunoglobulin library (e.g., an antibody phage display library) with the polypeptide of interest, e.g., an ABL1 fusion polypeptide provided herein or a fragment thereof. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display libraries can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; and Griffiths et al. (1993) EMBO J. 12:725-734. In some embodiments, monoclonal antibodies or antibody fragments of the disclosure are recombinant, such as chimeric or humanized monoclonal antibodies or antibody fragments, comprising both human and non-human portions. Such chimeric and/or humanized monoclonal antibodies or antibody fragments can be produced by recombinant DNA techniques known in the art, for example, using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559; Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060. In some embodiments, a monoclonal antibody or antibody fragment of the disclosure is a human monoclonal antibody or antibody fragment. In some embodiments, human monoclonal antibodies or antibody fragments are prepared using methods known in the art, e.g., using transgenic mice which are incapable of expressing endogenous immunoglobulin heavy and light chains genes, but which can express human heavy and light chain genes. For an overview of this technology for producing human antibodies, see Lonberg and Huszar (1995) Int. Rev. Immunol. 13:65-93. For a detailed discussion of this technology for producing human antibodies and human monoclonal antibodies or fragments thereof, and protocols for producing such antibodies or fragments thereof, see, e.g., U.S. Pat. Nos. 5,625,126; 5,633,425; 5,569,825; 5,661,016; and 5,545,806.

In some embodiments, the antibody or antibody fragment of the disclosure is an isolated antibody or antibody fragment, which has been separated from a component of its natural environment or a cell culture used to produce the antibody or antibody fragment. In some embodiments, an antibody or antibody fragment of the disclosure is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods.

In some embodiments, an antibody or antibody fragment of the disclosure can be used to isolate an ABL1 fusion polypeptide provided herein, or a fragment thereof, by standard techniques, such as affinity chromatography or immunoprecipitation. In some embodiments, an antibody or antibody fragment of the disclosure can be used to detect an ABL1 fusion polypeptide provided herein, or a fragment thereof, e.g., in a tissue sample, cellular lysate, or cell supernatant, in order to evaluate the level and/or pattern of expression of the ABL1 fusion polypeptide. Detection can be facilitated by coupling the antibody or antibody fragment to a detectable substance. Thus, in some embodiments, an antibody or antibody fragment of the disclosure is coupled to a detectable substance, such as enzymes, prosthetic groups, fluorescent materials, luminescent materials, bioluminescent materials, and radioactive materials. Non-limiting examples of suitable enzymes include, e.g., horseradish peroxidase, alkaline phosphatase, β-galactosidase, or acetylcholinesterase; examples of suitable prosthetic group complexes include, e.g., streptavidin/biotin and avidin/biotin; examples of suitable fluorescent materials include, e.g., umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, e.g., luciferase, luciferin, and aequorin; and examples of suitable radioactive materials include, e.g., ¹²⁵I, ¹³¹I, ³⁵S or ³H.

An antibody or antibody fragment of the disclosure may also be used diagnostically, e.g., to detect and/or monitor protein levels (e.g., protein levels of an ABL1 fusion polypeptide provided herein) in tissues or body fluids (e.g., in a tumor cell-containing tissue or body fluid), e.g., according to the methods provided herein.

In certain embodiments, an antibody or antibody fragment provided herein has a dissociation constant (Kd) of ≤1 μM, ≤100 nM, ≤10 nM, ≤1 nM, ≤0.1 nM, ≤0.01 nM, or ≤0.001 nM (e.g., 10⁻⁸ M or less, e.g., from 108 M to 10⁻¹³ M, e.g., from 109 M to 10⁻¹³ M) for its target, e.g., an ABL1 fusion polypeptide of the disclosure. Methods of measuring antibody or antibody fragment affinity (e.g., Kd) are known in the art, and include, without limitation, a radiolabeled antigen binding assay (RIA) and a BIACORE® surface plasmon resonance (SPR) assay. In some embodiments, antibody affinity (e.g., Kd) is determined using the Fab version of an antibody of the disclosure and its antigen (e.g., an ABL1 fusion polypeptide provided herein), e.g., using RIA or SPR.

In certain embodiments, an antibody fragment provided herein is a Fab, Fab′, Fab′-SH, F(ab′)₂, Fv, single-chain antibody molecule (e.g., scFv), scFv-Fc fragment, and other fragments described herein or known in the art. In certain embodiments, an antibody fragment provided herein is a diabody. Diabodies are antibody fragments with two antigen-binding sites that may be bivalent or bispecific. In certain embodiments, an antibody fragment provided herein is a triabody or a tetrabody. In certain embodiments, an antibody fragment provided herein is a single-domain antibody fragment. Single-domain antibody fragments comprise all or a portion of the heavy chain variable domain or all or a portion of the light chain variable domain of an antibody. In certain embodiments, a single-domain antibody fragment is a human single-domain antibody fragment.

Antibody fragments can be made by various techniques, including but not limited to proteolytic digestion of an intact antibody, as well as production by recombinant host cells (e.g., E. coli or phage), as known in the art and as described herein.

In certain embodiments, an antibody or antibody fragment provided herein is a chimeric antibody. In one example, a chimeric antibody or antibody fragment comprises a non-human variable region (e.g., a variable region derived from a mouse, rat, hamster, rabbit, or non-human primate, such as a monkey), and a human constant region or portions thereof. In a further example, a chimeric antibody or antibody fragment is a “class switched” antibody or antibody fragment, in which the class or subclass of the antibody or antibody fragment has been changed from that of the parent antibody or antibody fragment. Chimeric antibodies include antigen-binding fragments thereof.

In certain embodiments, a chimeric antibody or antibody fragment is a humanized antibody or antibody fragment. Typically, a non-human antibody or antibody fragment is humanized to reduce immunogenicity to humans, while retaining the specificity and affinity of the parental non-human antibody or antibody fragment. Generally, a humanized antibody or antibody fragment comprises one or more variable domains in which HVRs, e.g., CDRs, (or portions thereof), are derived from a non-human antibody, and framework regions (FRs) (or portions thereof) are derived from human antibody sequences. A humanized antibody or antibody fragment optionally will also comprise at least a portion of a human constant region. In some embodiments, some FR residues in a humanized antibody or antibody fragment are substituted with corresponding residues from a non-human antibody or antibody fragment (e.g., the antibody or antibody fragment from which the HVR residues are derived), e.g., to restore or improve specificity or affinity. Humanized antibodies or antibody fragments, and methods of making them are known in the art. Human framework regions that may be used for humanization include but are not limited to: framework regions selected using the “best-fit” method; framework regions derived from the consensus sequence of human antibodies of a particular subgroup of light or heavy chain variable regions; human mature (somatically mutated) framework regions or human germline framework regions; and framework regions derived from screening FR libraries.

In certain embodiments, an antibody or antibody fragment provided herein is a human antibody. Human antibodies or antibody fragments can be produced using various techniques known in the art. For example, human antibodies or antibody fragments may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or are present extrachromosomally or integrated randomly into the animal's chromosomes. In such transgenic animals, e.g., mice, the endogenous immunoglobulin loci have generally been inactivated. Human variable regions from intact antibodies generated by such animals may be further modified, e.g., by combining with a different human constant region. Human antibodies or antibody fragments can also be made by hybridoma-based methods known in the art, e.g., using known human myeloma and mouse-human heteromyeloma cell lines for the production of human monoclonal antibodies. Human antibodies or antibody fragments may also be generated by isolating Fv clone variable domain sequences selected from human-derived phage display libraries. Such variable domain sequences may then be combined with a desired human constant domain or portions thereof. Techniques for selecting human antibodies or fragments thereof from libraries are known in the art and described herein.

Antibodies or antibody fragments of the disclosure may be isolated by screening combinatorial libraries for antibodies or fragments thereof with the desired activity or activities. For example, a variety of methods are known in the art for generating phage display libraries and screening such libraries for antibodies or antibody fragments possessing the desired binding characteristics. In certain phage display methods, repertoires of VH and VL genes are separately cloned by polymerase chain reaction (PCR) and recombined randomly in phage libraries, which can then be screened for antigen-binding phage. Phage typically display antibody fragments, either as single-chain Fv (scFv) fragments or as Fab fragments. Libraries from immunized sources provide high-affinity antibodies to the immunogen without the requirement of constructing hybridomas. Alternatively, a naive antibody repertoire can be cloned (e.g., from human) to provide a single source of antibodies or fragments thereof to a wide range of non-self and also self antigens without any immunization. Naive libraries can also be made synthetically by cloning un-rearranged V-gene segments from stem cells, and using PCR primers containing random sequences to amplify the highly variable CDR3 regions and to accomplish rearrangement in vitro. Antibodies or antibody fragments isolated from human antibody libraries are considered human antibodies or human antibody fragments herein.

In certain embodiments, an antibody or antibody fragment provided herein is multispecific, e.g., bispecific. Multispecific antibodies or antibody fragments are monoclonal antibodies or antibody fragments that have binding specificities for at least two different sites or at least two different antigens. For example, one of the binding specificities can be to an ABL1 fusion polypeptide of the disclosure, and the other can be to any other antigen. Techniques for making multispecific antibodies or antibody fragments are known in the art and include, but are not limited to, recombinant co-expression of two immunoglobulin heavy chain-light chain pairs having different specificities, and “knob-in-hole” engineering. Multispecific antibodies or antibody fragments may also be made by engineering electrostatic steering effects (e.g., by introducing mutations in the constant region) for making heterodimeric Fcs; cross-linking two or more antibodies or fragments; using leucine zippers to produce bispecific antibodies or antibody fragments; using “diabody” technology for making bispecific antibody fragments; using single-chain Fv (scFv) dimers; and preparing trispecific antibodies or antibody fragments. Engineered antibodies or antibody fragments with three or more functional antigen binding sites, including “Octopus antibodies,” are also included in the disclosure. Antibodies or antibody fragments of the disclosure also include “Dual Acting FAbs” or “DAF,” e.g., comprising an antigen binding site that binds to an ABL1 fusion polypeptide of the disclosure as well as another, different antigen.

In certain embodiments, amino acid sequence variants of the antibodies or antibody fragments provided herein are contemplated. For example, it may be desirable to improve the binding affinity and/or other biological properties of the antibody or antibody fragment. Amino acid sequence variants of an antibody or antibody fragment of the disclosure may be prepared by introducing appropriate modifications into the nucleotide sequence encoding the antibody or antibody fragment, or by peptide synthesis. Such modifications include, for example, deletions, and/or insertions, and/or substitutions of residues within the amino acid sequences of the antibody or antibody fragment. Any combination of deletions, insertions, and substitutions can be made to arrive at the final antibody or antibody fragment, provided that the final antibody or antibody fragment possesses the desired characteristics, e.g., antigen-binding.

In certain embodiments, antibody or antibody fragment variants having one or more amino acid substitutions are provided. Sites of interest for substitutional mutagenesis include the HVRs and FRs. Amino acid substitutions may be introduced into an antibody or antibody fragment of interest, and the products may be screened for a desired activity, e.g., retained/improved antigen binding, decreased immunogenicity, or improved or reduced antibody-dependent cell-mediated cytotoxicity (ADCC) and/or complement-dependent cytotoxicity (CDC).

In certain embodiments, an antibody or antibody fragment of the present disclosure is altered to increase or to decrease the extent to which the antibody is glycosylated. Addition or deletion of glycosylation sites to an antibody or antibody fragment may be conveniently accomplished by altering the amino acid sequence of the antibody or antibody fragment, such that one or more glycosylation sites is created or removed. Antibody or antibody fragment variants having bisected oligosaccharides are further provided, e.g., in which a biantennary oligosaccharide attached to the Fc region of the antibody or antibody fragment is bisected by GlcNAc. In some embodiments, antibody or antibody fragment variants of the disclosure may have increased fucosylation. In some embodiments, antibody or antibody fragment variants of the disclosure may have reduced fucosylation. In some embodiments, antibody or antibody fragment variants of the disclosure may have improved ADCC function. In some embodiments, antibody or antibody fragment variants of the disclosure may have decreased ADCC function. Antibody or antibody fragment variants with at least one galactose residue in the oligosaccharide attached to the Fc region are also provided. Such variants may have improved CDC function. In some embodiments, antibody or antibody fragment variants of the disclosure may have increased or decreased CDC function.

In certain embodiments, one or more amino acid modifications may be introduced into the Fc region of an antibody or antibody fragment of the present disclosure, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the present disclosure contemplates an antibody or antibody fragment variant that possesses some but not all effector functions, which make it a desirable candidate for applications in which the half-life of the antibody or antibody fragment in vivo is important, yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody or antibody fragment lacks Fc-gamma-R binding (hence likely lacking ADCC activity), but retains FcRn binding ability. The primary cells that mediate ADCC, e.g., NK cells, express Fc-gamma-RIII only, whereas monocytes express Fc-gamma-RI, Fc-gamma-RII and Fc-gamma-RIII. Antibodies or antibody fragments with reduced effector function include those with substitution of one or more of Fc region residues 238, 265, 269, 270, 297, 327 and 329. Such Fc mutants include Fc mutants with substitutions at two or more of amino acid positions 265, 269, 270, 297 and 327, including the so-called “DANA” Fc mutant with substitutions of residues 265 and 297 to alanine. Antibody or antibody fragment variants with improved or diminished binding to FcRs are also included in the disclosure. In certain embodiments, an antibody or antibody fragment variant comprises an Fc region with one or more amino acid substitutions that improve ADCC, e.g., substitutions at positions 298, 333, and/or 334 of the Fc region. In some embodiments, numbering of Fc region residues is according to EU numbering of residues. In some embodiments, alterations are made in the Fc region that result in altered (i.e., either improved or diminished) Clq binding and/or CDC. In some embodiments, antibodies or antibody fragments of the disclosure have increased half-lives and improved binding to the neonatal Fc receptor (FcRn), e.g., comprising one or more substitutions that improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434. See, also, Duncan & Winter, Nature 322:738-40 (1988); U.S. Pat. Nos. 5,648,260; 5,624,821; and WO 94/29351 for other examples of Fc region variants.

In certain embodiments, an antibody or antibody fragment provided herein is cysteine-engineered, e.g., “thioMAb,” in which one or more residues of the antibody or antibody fragment are substituted with cysteine residues. In some embodiments, the substituted residues occur at accessible sites of the antibody or antibody fragment. By substituting those residues with cysteine, reactive thiol groups are thereby positioned at accessible sites of the antibody or antibody fragment, and may be used to conjugate the antibody or antibody fragment to other moieties, such as drug moieties or linker-drug moieties, e.g., to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine-engineered antibodies or antibody fragments may be generated using any suitable method known in the art.

In some embodiments, an antibody or antibody fragment provided herein comprises a label or a tag. In some embodiments, the label or tag is a radiolabel, a fluorescent label, an enzymatic label, a sequence tag, biotin, or other ligands. Examples of labels or tags include, but are not limited to, 6xHis-tag, biotin-tag, Glutathione-S-transferase (GST)-tag, green fluorescent protein (GFP)-tag, c-myc-tag, FLAG-tag, Thioredoxin-tag, Glu-tag, Nus-tag, V5-tag, calmodulin-binding protein (CBP)-tag, Maltose binding protein (MBP)-tag, Chitin-tag, alkaline phosphatase (AP)-tag, HRP-tag, Biotin Caboxyl Carrier Protein (BCCP)-tag, Calmodulin-tag, S-tag, Strep-tag, haemoglutinin (HA)-tag, digoxigenin (DIG)-tag, DsRed, RFP, Luciferase, Short Tetracysteine Tags, Halo-tag, and Nus-tag. In some embodiments, the label or tag comprises a detection agent, such as a fluorescent molecule or an affinity reagent or tag.

In some embodiments, an antibody or antibody fragment provided herein is conjugated to a drug molecule, e.g., an anti-cancer agent described herein, or a cytotoxic agent such as mertansine or monomethyl auristatin E (MMAE).

In certain embodiments, an antibody or antibody fragment provided herein may be further modified to contain additional nonproteinaceous moieties. Such moieties may be suitable for derivatization of the antibody or antibody fragment, e.g., including but not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyamino acids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, polyethylene glycol propionaldehyde, and mixtures thereof. The polymers may be of any molecular weight, and may be branched or unbranched. The number of polymers attached to the antibody or antibody fragment may vary, and if more than one polymer is attached, the polymers can be the same or different molecules. In general, the number and/or type of polymers used for derivatization can be determined based on considerations including, but not limited to, the particular properties or functions of the antibody or antibody fragment to be improved, or whether the antibody or antibody fragment derivative will be used in a therapy under defined conditions. In some embodiments, provided herein are antibodies or antibody fragments conjugated to carbon nanotubes, e.g., for use in methods to selectively heat the antibody or antibody fragment using radiation to a temperature at which cells proximal to the antibody or antibody fragment are killed.

### (iv) Samples

A variety of materials can be the source of, or serve as, samples for use in any of the methods of the disclosure, such as the methods for detection of an ABL1 fusion nucleic acid molecule or an ABL1 fusion polypeptide of the disclosure, or fragments thereof.

For example, the sample can be, or be derived from: solid tissue such as from a fresh, frozen and/or preserved organ, tissue sample, biopsy (e.g., tumor, tissue or liquid biopsy), resection, smear, or aspirate; scrapings; bone marrow or bone marrow specimens; a bone marrow aspirate; blood or any blood constituents; blood cells; bodily fluids such as cerebrospinal fluid, amniotic fluid, urine, saliva, sputum, peritoneal fluid or interstitial fluid; pleural fluid; ascites; tissue or fine needle biopsy samples; surgical specimens; cell-containing body fluids; free-floating nucleic acids; feces; lymph; gynecological fluids; skin swabs; vaginal swabs; oral swabs; nasal swabs; washings or lavages such as ductal lavages or bronchoalveolar lavages; cells from any time in gestation or development of an individual; cells from a cancer or tumor; other body fluids, secretions, and/or excretions, and/or cells therefrom. In some embodiments, a sample is or comprises cells obtained from an individual. In some embodiments, the sample is or is derived from blood or blood constituents, e.g., obtained from a liquid biopsy. In some embodiments, the sample is or is derived from a tumor sample. In some embodiments, the sample is or comprises biological tissue or fluid. In some embodiments, the sample can contain compounds that are not naturally intermixed with the source of the sample in nature, such as preservatives, anticoagulants, buffers, fixatives, nutrients, antibiotics, and the like. In some embodiments, the sample is preserved as a frozen sample or as a formaldehyde- or paraformaldehyde-fixed paraffin-embedded (FFPE) tissue preparation. In some embodiments, the sample comprises circulating tumor cells (CTCs).

In one embodiment, the sample comprises one or more cells associated with a tumor, e.g., tumor cells or tumor-infiltrating lymphocytes (TIL). In one embodiment, the sample includes one or more premalignant or malignant cells. In one embodiment, the sample is acquired from a hematologic malignancy (or pre-malignancy), e.g., a hematologic malignancy (or pre-malignancy) described herein. In one embodiment, the sample is acquired from a cancer, such as a cancer described herein. In some embodiments, the sample is acquired from a solid tumor, a soft tissue tumor or a metastatic lesion. In other embodiments, the sample includes tissue or cells from a surgical margin. In one embodiment, the sample is or is acquired from a liquid biopsy of blood, plasma, cerebrospinal fluid, sputum, stool, urine, or saliva. In some embodiments, the sample includes cell-free DNA (cfDNA) and/or circulating tumor DNA (ctDNA), e.g., from a biopsy of blood, plasma, cerebrospinal fluid, sputum, stool, urine, or saliva. In another embodiment, the sample includes one or more circulating tumor cells (CTCs) (e.g., a CTC acquired from a blood sample). In one embodiment, the sample is a cell not associated with a tumor or cancer, e.g., a non-tumor or non-cancer cell or a peripheral blood lymphocyte.

In some embodiments, a sample is a primary sample obtained directly from a source of interest by any appropriate means. For example, in some embodiments, a primary biological sample is obtained by a method chosen from biopsy (e.g., fine needle aspiration or tissue biopsy), surgery, or collection of body fluid (e.g., blood, lymph, or feces). In some embodiments, as will be clear from context, a sample refers to a preparation that is obtained by processing (e.g., by removing one or more components of and/or by adding one or more agents to) a primary sample. Such a processed sample may comprise, for example, nucleic acids (e.g., for use in any of the methods for detection of ABL1 fusion nucleic acid molecules provided herein) or proteins (e.g., for use in any of the methods for detection of ABL1 fusion polypeptides provided herein) extracted from a sample or obtained by subjecting a primary sample to techniques such as amplification methods, reverse transcription of mRNA, or isolation and/or purification of certain components such as nucleic acids and/or proteins.

In some embodiments, the sample comprises nucleic acids, e.g., genomic DNA, cDNA, or mRNA. In some embodiments, the sample comprises cell-free DNA (cfDNA). In some embodiments, the sample comprises cell-free RNA (cfRNA). In some embodiments, the sample comprises circulating tumor DNA (ctDNA). In certain embodiments, the nucleic acids are purified or isolated (e.g., removed from their natural state). In some embodiments, the sample comprises tumor or cancer nucleic acids, such as nucleic acids from a tumor or cancer sample, e.g., genomic DNA, RNA, or cDNA derived from RNA, or from a liquid biopsy, e.g., ctDNA from blood, plasma, cerebrospinal fluid, sputum, stool, urine, or saliva. In certain embodiments, a tumor or cancer nucleic acid sample, or a ctDNA sample, is purified or isolated (e.g., it is removed from its natural state).

In some embodiments, the sample comprises tumor or cancer proteins or polypeptides, such as proteins or polypeptides from a tumor or a cancer sample, or from a liquid biopsy, e.g., from blood, plasma, cerebrospinal fluid, sputum, stool, urine, or saliva. In certain embodiments, the proteins or polypeptides are purified or isolated (e.g., removed from their natural state).

In some embodiments, the sample is obtained from an individual having a cancer, such as a cancer described herein. In some embodiments, the sample comprises an ABL1 fusion nucleic acid molecule or an ABL1 fusion polypeptide of the disclosure, or fragments or portions thereof. In some embodiments, the methods provided herein comprise obtaining one or more samples from the individual (e.g., the individual having a cancer). In some embodiments, the one or more samples are obtained or derived from a cancer (e.g., a cancer in an individual). In some embodiments, the one or more samples comprise at least 20% tumor cell nuclear area.

In some embodiments, the sample is a control sample or a reference sample, e.g., not containing an ABL1 fusion nucleic acid molecule or an ABL1 fusion polypeptide described herein. In certain embodiments, the reference sample is purified or isolated (e.g., it is removed from its natural state). In certain embodiments, the reference or control sample comprises a wild type or a non-mutated nucleic acid molecule or polypeptide counterpart to any of the ABL1 fusion nucleic acid molecules or ABL1 fusion polypeptides described herein. In other embodiments, the reference sample is from a non-tumor or cancer sample, e.g., a blood control, a normal adjacent tumor (NAT), or any other non-cancerous sample from the same or a different individual.

In some embodiments, an ABL1 fusion nucleic acid molecule of the disclosure is detected in a sample comprising genomic or subgenomic DNA fragments, or RNA (e.g., mRNA), isolated from a sample, e.g., a tumor or cancer sample, a normal adjacent tissue (NAT) sample, a tissue sample, or a blood, plasma, cerebrospinal fluid, sputum, stool, urine, or saliva sample obtained from an individual. In some embodiments, the sample comprises cDNA derived from an mRNA sample or from a sample comprising mRNA. In some embodiments, an ABL1 fusion nucleic acid molecule of the disclosure is detected in a sample comprising cell-free DNA (cfDNA), cell-free RNA, and/or circulating tumor DNA (ctDNA). In some embodiments, an ABL1 fusion nucleic acid molecule of the disclosure is detected in a sample comprising cell-free DNA (cfDNA) and/or circulating tumor DNA (ctDNA). In some embodiments, an ABL1 fusion nucleic acid molecule of the disclosure is detected in a sample comprising circulating tumor DNA (ctDNA).

In some embodiments, any of the methods of the present disclosure comprise acquiring knowledge of or detecting any of the biomarkers described herein (e.g., an ABL1 fusion nucleic acid molecule or an ABL1 fusion polypeptide, and/or one or more alterations in one or more genes) in one or more samples (e.g., as described above) obtained from an individual (e.g., an individual having a cancer). In some embodiments, the samples used to acquire knowledge of or detect any of the biomarkers described herein (e.g., an ABL1 fusion nucleic acid molecule or an ABL1 fusion polypeptide, and/or one or more alterations in one or more genes) are the same sample (i.e., one or more, or all, of an ABL1 fusion nucleic acid molecule, an ABL1 fusion polypeptide, and one or more alterations in one or more genes are detected or determined in one sample). In some embodiments, the samples used to acquire knowledge of or detect any of the biomarkers described herein (e.g., an ABL1 fusion nucleic acid molecule or an ABL1 fusion polypeptide, and/or one or more alterations in one or more genes) comprise more than one sample (e.g., some of the biomarkers may be detected or determined in one sample, and some of the biomarkers may be detected or determined in another sample). For example, in some embodiments, an ABL1 fusion nucleic acid molecule or fusion polypeptide, may be detected in one sample, and one or more alterations in one or more genes may be detected or determined in an another sample. In another example, an ABL1 fusion nucleic acid molecule, may be detected in one sample, and an ABL1 fusion polypeptide may be detected or determined in an another sample.

### C. Anti-Cancer Therapies

Certain aspects of the present disclosure relate to anti-cancer therapies, as well as methods for identifying an individual having a cancer who may benefit from a treatment comprising an anti-cancer therapy; selecting a treatment for an individual having a cancer; identifying one or more treatment options for an individual having a cancer; predicting survival of an individual having a cancer; treating or delaying progression of cancer; monitoring, evaluating or screening an individual having a cancer; detecting the presence or absence of a cancer in an individual; monitoring progression or recurrence of a cancer in an individual; or identifying a candidate treatment for a cancer in an individual in need thereof. The present disclosure also provides uses for anti-cancer therapies (e.g., in methods of treating or delaying progression of cancer in an individual, or in methods for manufacturing a medicament for treating or delaying progression of cancer). In some instances, the methods of the disclosure can include administering a treatment comprising an anti-cancer therapy or applying a treatment comprising an anti-cancer therapy to an individual based on a generated molecular and/or sequencing mutation profile. An anti-cancer therapy can refer to an agent or a compound that is effective in the treatment of cancer cells. Examples of anti-cancer agents, anti-cancer compounds, or anti-cancer therapies include, but are not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

In some embodiments, an anti-cancer therapy of the disclosure is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer comprising an ABL1 fusion nucleic acid molecule or an ABL1 fusion polypeptide of the disclosure, a treatment for cancer being tested in a clinical trial, a targeted therapy, a treatment being tested in a clinical trial for cancer comprising an ABL1 fusion nucleic acid molecule or an ABL1 fusion polypeptide of the disclosure, or any combination thereof, e.g., a described in further detail below. In some embodiments, the anti-cancer therapy is an ABL1-targeted therapy. In some embodiments, the anti-cancer therapy, e.g., the ABL1-targeted therapy, is a kinase inhibitor, such as a kinase inhibitor described herein or known in the art. In some embodiments, the kinase inhibitor is a tyrosine kinase inhibitor. In some embodiments, the kinase inhibitor inhibits the kinase activity of an ABL1 polypeptide. In some embodiments, the kinase inhibitor is a multi-kinase inhibitor or an ABL1-specific inhibitor known in the art or described herein. In some embodiments, the nucleic acid inhibits the expression of an ABL1 fusion nucleic acid molecule or an ABL1 fusion polypeptide of the disclosure.

In some embodiments, the anti-cancer therapy, e.g., the ABL1-targeted therapy, is imatinib, nilotinib, dasatinib, ponatinib, bosutinib, asciminib, olverembatinib, radotinib or vodobatinib, or any combination thereof. The BCR-ABL fusion protein is the best-studied ABL1 alteration and results in an activated ABL kinase; therapies to inhibit activated ABL1 have focused on BCR-ABL-positive hematological malignancies (Lim et al. (2005). Annals of the Academy of Medicine, Singapore, 34(9), 533-538; Konopka et al., PNAS 82(6), 1810-1814). Tyrosine kinase inhibitors (TKIs) such as imatinib, nilotinib, dasatinib, ponatinib, bosutinib, and asciminib have been approved to treat certain hematological malignancies with BCR-ABL fusions. Treatment with these therapies has been correlated with increased responses for patients with ALL or CML compared with treatment regimens lacking TKIs (Fielding (2011). Hematology. American Society of Hematology. Education Program, 2011, 231-237; Santos et al. (2011). Current hematologic malignancy reports, 6(2), 96-103; Reddy et al. (2012). Genes & cancer, 3 (5-6), 447-454; Soverini et al. (2011). Blood, 118(5), 1208-1215; Keller-V Amsberg et al. (2012). Expert review of anticancer therapy, 12(9), 1121-1127). Patients with Philadelphia chromosome-positive chronic-phase or accelerated-phase chronic myeloid leukemia (CML) resistant or intolerant to prior ABL tyrosine kinase inhibitors may benefit from asciminib (Réa et al. (2021). Blood, 138(21), 2031-2041; Hughes et al. (2019). The New England journal of medicine, 381(24), 2315-2326), olverembatinib (Jiang et al., 2020; ASH Abstract 651), or vodobatinib (Cortes et al., 2020; ASH Abstract 652).

In some embodiments, the anti-cancer therapy, e.g., the ABL1-targeted therapy, is bosutinib. In clinical studies, bosutinib facilitated remissions for patients with CML, including a 73% response for those who had failed prior imatinib or nilotinib therapies (Khoury et al. (2012). Blood, 119(15), 3403-3412; Amsberg et al. (2013). Biologics: targets & therapy, 7, 115-122; Komeno et al. (2017). Oncology letters, 13(6), 4285-4289; Valent et al. (2017). Oncotarget, 8(14), 23061-23072). Two Phase 3 studies for patients with newly diagnosed, chronic phase CML (CP-CML) reported significantly higher rates of major molecular response (MMR) at 12 months following treatment with bosutinib versus imatinib (BELA, 41% vs. 27%; and BFORE, 47% and 37%; Cortes et al. (2012). Journal of clinical oncology: official journal of the American Society of Clinical Oncology, 30(28), 3486-3492; Cortes et al. (2018). Journal of clinical oncology: official journal of the American Society of Clinical Oncology, 36(3), 231-237); trends for higher cumulative MMR with bosutinib than imatinib persisted in follow-up studies of both the BELA (59% vs. 49%, >24 months) (Brummendor et al. (2015). British journal of haematology, 168(1), 69-81) and BFORE (74% vs. 65%, 5-year) trials (Brummendorf et al., 2020; ASH Abstract 46). For patients in the BFORE trial with e13a2/e14a2 Ph+ CP-CML, more complete cytogenetic responses (CCyRs) were also achieved with bosutinib than imatinib (77.2% vs. 66.4%, p=0.0075) (Cortes et al. (2018). Journal of clinical oncology: official journal of the American Society of Clinical Oncology, 36 (3), 231-237). In an 8-year follow-up from a Phase 1/2 study of bosutinib as second-line therapy for patients with Ph+CP-CML who were resistant (IM-R) or intolerant (IM-I) to first-line imatinib, 60% of patients experienced a major cytogenetic response (MCyR), and 49% (IM-R) and 51% (IM-I) achieved CCyR (Brümmendorf et al., 2017; ASH Abstract 900); for imatinib- and nilotinib- or dasatinib-refractory or intolerant patients treated in the third- or fourth-line with bosutinib in this study, a MCyR of 40% at 4 years of follow-up was achieved (Cortes et al. (2016). American journal of hematology, 91(12), 1206-1214). In a Phase 1/2 study for Japanese patients with Ph+ CP-CML, second- and third-line treatment with bosutinib effected MCyRs and MMRs of 73% and 70% and of 53% and 40%, respectively (Takahashi et al. (2017). International journal of hematology, 106(3), 398-410). Similarly, high rates of CCyRs and MMRs (62% and 54%, respectively) were observed in a retrospective study of heavily pretreated patients (85%, 45/53) with CML who were treated with bosutinib in a real-world setting (Apperley et al., 2016; ASH Abstract 5435). An elderly patient whose CML transformed to B-ALL achieved a complete hematologic response after 14 months of fourth-line bosutinib treatment (Atilla et al. (2015). Case reports in hematology, 2015, 689423).

In some embodiments, the anti-cancer therapy, e.g., the ABL1-targeted therapy, is dasatinib. In a 7-year analysis of the CA180-034 pivotal Phase 3 trial, dasatinib in patients with imatinib-resistant Philadelphia-chromosome-positive (Ph+) chronic-phase CML (CP-CML) elicited 65% OS, 42% PFS, and 46% major molecular response (Shah et al. (2016). American journal of hematology, 91(9), 869-874). In a Phase 3 clinical trial, patients with CP-CML receiving dasatinib demonstrated superior cytogenetic and molecular responses to patients receiving imatinib, with earlier median time to complete cytogenetic response (CCyR) (3 vs. 6 months) and lower incidence of transformation to blast-phase CML (Cortes et al. (2016). Journal of clinical oncology: official journal of the American Society of Clinical Oncology, 34(20), 2333-2340; Jabbour et al. (2014). Blood, 123(4), 494-500; Kantarjian et al. (2012). Blood, 119(5), 1123-1129; Kantarjian et al. (2010). The New England journal of medicine, 362(24), 2260-2270). In a Phase 2 study of pediatric patients with newly diagnosed (ND) or imatinib-resistant (IR) CP-CML, dasatinib treatment achieved significant clinical benefit at 12 months, including complete CCyR of 76% (IR) to 92% (ND) and major molecular response of 41% (IR) to 52% (ND), and a 48-month PFS estimated at 78% (IR) to 93% (ND); longer PFS was observed for ND patients with BCR-ABL1≤10% vs. those with BCR-ABL1>10% at 3 months (p<0.001; HR, 0.10) (Gore et al. (2018). Journal of clinical oncology: official journal of the American Society of Clinical Oncology, 36(13), 1330-1338). A Phase 1 trial of dasatinib for pediatric patients with relapsed or refractory leukemia observed major hematologic responses (MaHRs) for 47% (8/17) of cases with advanced-phase CML or Ph+ acute lymphoblastic leukemia (ALL) and a median MaHR duration of 4.4 months (Zwaan et al. (2013). Journal of clinical oncology: official journal of the American Society of Clinical Oncology, 31(19), 2460-2468).

In some embodiments, the anti-cancer therapy, e.g., the ABL1-targeted therapy, is imatinib. Imatinib is approved to treat chronic phase (CP), accelerated phase, or blast crisis Philadelphia chromosome-positive chronic myeloid leukemia (Ph+ CML) (Cohen et al. (2002). Clinical cancer research: an official journal of the American Association for Cancer Research, 8(5), 935-942; Druker et al. (2001). The New England journal of medicine, 344(14), 1031-1037) and is indicated in the first-line setting for Ph+ CML-CP, particularly in low-risk patients, according to the NCCN guidelines (v3.2020). In the Phase 3 IRIS study of first-line imatinib versus interferon alpha and cytarabine in Ph+ CML-CP, the 10-year OS rate for imatinib was 83.3%, with 82.8% of patients achieving a complete cytogenetic response (CCyR) (Hochhaus et al. (2017). The New England journal of medicine, 376(10), 917-927; Druker et al. (2006). The New England journal of medicine, 355(23), 2408-2417; O'Brien et al. (2003). The New England journal of medicine, 348(11), 994-1004; Hughes et al. (2003). The New England journal of medicine, 349(15), 1423-1432). Similar outcomes have been reported for other long-term imatinib studies (Kalmanti et al. (2015). Leukemia, 29(5), 1123-1132; Gambacorti-Passerini (2011). Journal of the National Cancer Institute, 103(7), 553-561). Retrospective comparison of the imatinib cohort from IRIS with prospective historical outcomes reported significantly improved 3-year OS (92% vs. 84%) and CCyR rates (81% vs. 32%) for imatinib as compared with interferon alpha and cytarabine (Roy et al. (2006). Blood, 108 (5), 1478-1484). Studies examining whether patients with sustained imatinib response can safely discontinue treatment have reported molecular relapse rates of 49%-61% (Etienne et al. (2017). Journal of clinical oncology: official journal of the American Society of Clinical Oncology, 35(3), 298-305; Nicolini et al. (2019). Clinical cancer research: an official journal of the American Association for Cancer Research, 25(22), 6606-6613; Ross et al. (2013). Blood, 122(4), 515-522), with longer TKI duration and higher residual disease burden linked to higher risk of relapse (Nicolini et al. (2019). Clinical cancer research: an official journal of the American Association for Cancer Research, 25(22), 6606-6613). Most relapses occurred within 6 months of imatinib discontinuation, and patients who relapsed remained sensitive to TKI rechallenge (Etienne et al. (2017). Journal of clinical oncology: official journal of the American Society of Clinical Oncology, 35(3), 298-305; Ross et al. (2013). Blood, 122(4), 515-522). Phase 3 studies have compared imatinib with other TKIs in the first-line for CML-CP and have reported superior molecular response rates for nilotinib (Hochhaus et al. (2016). Leukemia, 30(5), 1044-1054; Hughes et al. (2014). Blood, 123 (9), 1353-1360; Larson et al. (2012). Leukemia, 26(10), 2197-2203; Kantarjian et al. (2011). The Lancet. Oncology, 12(9), 841-851; Saglio et al. (2010). The New England journal of medicine, 362(24), 2251-2259), bosutinib (Brümmendorf et al. (2015). British journal of haematology, 168(1), 69-81; Gambacorti-Passerini et al. (2014). American journal of hematology, 89(10), 947-953; Cortes et al. (2012). Journal of clinical oncology: official journal of the American Society of Clinical Oncology, 30(28), 3486-3492; Cortes et al. (2018). Journal of clinical oncology: official journal of the American Society of Clinical Oncology, 36(3), 231-237; Brümmendorf et al., 2020, ASH Abstract 46), or dasatinib (Cortes et al. (2016). Journal of clinical oncology: official journal of the American Society of Clinical Oncology, 34(20), 2333-2340; Jabbour et al. (2014). Blood, 123(4), 494-500; Kantarjian et al. (2012). Blood, 119 (5), 1123-1129; Kantarjian et al. (2010). The New England journal of medicine, 362(24), 2260-2270) as compared with imatinib.

In some embodiments, the anti-cancer therapy, e.g., the ABL1-targeted therapy, is nilotinib. A study investigating the use of nilotinib as first-line treatment for patients with Ph+ CML reported a 24-month major molecular response (MMR) rate of 83% (38/46) and progression-free survival and overall survival rates of 98% (Nishiwaki et al., 2016; ASH Abstract 1925). In the ENESTnd Phase 3 trial in patients with newly diagnosed chronic phase CML, nilotinib demonstrated a superior 5-year MMR rate (77% vs. 60%, p=0.0001) and 5-year deep molecular response rate (MR4.5) (52% vs. 31%, P=0.0001) in comparison to imatinib (Hochhaus et al. (2016). Leukemia, 30(5), 1044-1054). The ENEST freedom Phase 2 study assessing the potential for treatment-free remission (TFR) in adult patients with Ph+ CML treated with first-line nilotinib reported a TFR of 88%, with 52% of patients remaining in remission at 48 weeks after stopping nilotinib (Hochhaus et al. (2017). Leukemia, 31 (7), 1525-1531). Of those patients who did not remain in remission, 99% regained an MMR after reinitiating nilotinib treatment. Multiple studies have demonstrated that patients who achieved an MMR but did not experience an MR4.5 on imatinib treatment were able to achieve an MR4.5 after 24 months on second-line nilotinib treatment (MR4.5 rates of 42%-66%) (Shibiyama et al., 2016, ASH Abstract 1914; Rea et al., 2016, ASH Abstract 3094; Ritchie et al., 2016, ASH Abstract 3090).

In some embodiments, the anti-cancer therapy, e.g., the ABL1-targeted therapy, is ponatinib. The Phase 2 PACE study evaluating the efficacy of ponatinib for patients with chronic phase CML (CP-CML) who were resistant or intolerant to dasatinib or nilotinib or who had the T315I mutation (Cortes et al. (2013). The New England journal of medicine, 369(19), 1783-1796) reported 87% of patients maintained a major cytogenetic response (MCyR) and 74% maintained a major molecular response (MMR) by 2 years; 4-year complete CyR, MCyR, MMR, and molecular response 4.5 (MR4.5) rates of 54%, 59%, 39%, and 23%, respectively, were also observed (Cortes et al., 2016, ASCO Abstract 7013). Interim results from the related Phase 2 OPTIC trial to evaluate efficacy and safety with ponatinib over 3 different starting doses for patients with CP-CML observed 15-19% MMR across treatment groups (Cortes et al., 2020, ASCO Abstract 7502). Follow up analysis of the combined PACE and OPTIC trials observed high response rates and overall survival outcomes, with 5-year PFS of 52% and OS of 73% reported for patients enrolled in PACE, and a 2-year PFS of 81% and 93% OS for patients enrolled in OPTIC (Kantarjian et al., 2020, ASH Abstract 647). In a Phase 1 trial, 98% (42/43) of patients with refractory CP-CML had a complete hematological response, and 72% (31/43) had a major cytogenetic response to ponatinib (Cortes et al. (2012). The New England journal of medicine, 367(22), 2075-2088). A Phase 3 study comparing ponatinib with imatinib for patients with newly diagnosed CP-CML was terminated early because other trials had observed vascular adverse events with ponatinib (Lipton et al. (2016). The Lancet. Oncology, 17(5), 612-621). Preliminary data from this terminated study suggest ponatinib efficacy (major molecular responses at 12 months in 8/10 patients on ponatinib), although serious arterial occlusive events were significantly more frequent with ponatinib (6%) than with imatinib (1%) (Lipton et al. (2016). The Lancet. Oncology, 17(5), 612-621).

In some embodiments, the anti-cancer therapy, e.g., the ABL1-targeted therapy, is asciminib. Asciminib elicited superior efficacy over bosutinib in a Phase 3 trial for patients with Philadelphia chromosome-positive (Ph+) chronic myeloid leukemia (CML) in chronic phase without T315I mutation previously treated with 2 or more tyrosine kinase inhibitors, with a major molecular response (MMR) rate at week 24 of 26% and 13% for patients treated with asciminib and bosutinib, respectively; after adjusting for major cytogenetic response at baseline, the difference in MMR rate between treatment arms was 12% (p=0.029) (Réa et al. (2021). Blood, 138(21), 2031-2041). In a Phase 1 study evaluating asciminib in pretreated chronic-phase Ph+ CML, patients without T315I mutation achieved 70% (77/110) complete cytogenetic response (CCyR) and 48% (44/91) MMR by 12 months, and those with T315I mutation achieved 44% (11/25) CCyR and 24% (5/18) MMR by 12 months (Hughes et al. (2019). The New England journal of medicine, 381(24), 2315-2326). In the accelerated-phase CML cohort of this same study, 80% (4/5) of the patients with T315I mutation achieved complete hematologic response (CHR) and 1 patient achieved MMR by 11 weeks; 100% (3/3) of the patients without T315I mutation achieved CHR but not CCyR or MMR (Hughes et al. (2019). The New England journal of medicine, 381(24), 2315-2326).

In some embodiments, an anti-cancer therapy of the disclosure is administered in combination with an additional anti-cancer therapy. In some embodiments, the additional anti-cancer therapy is any anti-cancer therapy known in the art or described herein. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

In some embodiments, an anti-cancer therapy of the disclosure comprises a cyclin-dependent kinase (CDK) inhibitor, e.g., alone or in combination with an ABL1-targeted therapy. In some embodiments, the CDK inhibitor inhibits CDK4. In some embodiments, the CDK inhibitor inhibits Cyclin D/CDK4. In some embodiments, the CDK inhibitor is (a) a small molecule that inhibits one or more enzymatic activities of CDK4, (b) an antibody that inhibits one or more activities of CDK4 (e.g., by binding to and inhibiting one or more activities of CDK4, binding to and inhibiting expression of CDK4, and/or binding to and inhibiting one or more activities of a cell expressing CDK4, such as by inducing antibody-dependent cellular cytotoxicity, ADCC, or phagocytosis, ADCP), or (c) a nucleic acid that inhibits expression of CDK4 (e.g., an antisense oligonucleotide, miRNA, siRNA, morpholino, CRISPR-based therapeutic, and the like). In some embodiments, the CDK inhibitor inhibits CDK4 and CDK6. In some embodiments, the CDK inhibitor is a small molecule inhibitor of CDK4 (e.g., a competitive or non-competitive inhibitor). Non-limiting examples of CDK inhibitors include palbociclib, ribociclib, and abemaciclib, as well as pharmaceutically acceptable salts thereof.

In some embodiments, an anti-cancer therapy of the disclosure comprises a murine double minute 2 homolog (MDM2) inhibitor, e.g., alone or in combination with an ABL1-targeted therapy. In some embodiments, the MDM2 inhibitor is (a) a small molecule that inhibits one or more activities of MDM2 (e.g., binding to p53), (b) an antibody that inhibits one or more activities of MDM2 (e.g., by binding to and inhibiting one or more activities of MDM2, binding to and inhibiting expression of MDM2, and/or binding to and inhibiting one or more activities of a cell expressing MDM2, such as by inducing antibody-dependent cellular cytotoxicity, ADCC, or phagocytosis, ADCP), or (c) a nucleic acid that inhibits expression of MDM2 (e.g., an antisense oligonucleotide, miRNA, siRNA, morpholino, CRISPR-based therapeutic, and the like). In some embodiments, the MDM2 inhibitor is a small molecule inhibitor of MDM2 (e.g., a competitive or non-competitive inhibitor). Non-limiting examples of MDM2 inhibitors include nutlin-3a, RG7112, idasanutlin (RG7388), AMG-232, MI-63, MI-291, MI-391, MI-77301 (SAR405838), APG-115, DS-3032b, NVP-CGM097, and HDM-201 (siremadlin), as well as pharmaceutically acceptable salts thereof. In some embodiments, the MDM2 inhibitor inhibits or disrupts interaction between MDM2 and p53.

In some embodiments, an anti-cancer therapy of the disclosure comprises (alone or in combination with an ABL1-targeted therapy) one or more of an antimetabolite, DNA-damaging agent, or platinum-containing therapeutic (e.g., 5-azacitadine, 5-fluorouracil, acadesine, busulfan, carboplatin, cisplatin, chlorambucil, CPT-11, cytarabine, daunorubicin, decitabine, doxorubicin, etoposide, fludarabine, gemcitabine, idarubicin, radiation, oxaliplatin, temozolomide, topotecan, trabectedin, GSK2830371, or rucaparib); a pro-apoptotic agent (e.g., a BCL2 inhibitor or downregulator, SMAC mimetic, or TRAIL agonist such as ABT-263, ABT-737, oridonin, venetoclax, combination of venetoclax and an anti-CD20 antibody such as obinutuzumab or rituximab, 1396-11, ABT-10, SM-164, D269H/E195R, or rhTRAIL); a tyrosine kinase inhibitor (e.g., as described herein); an inhibitor of RAS, RAF, MEK, or the MAPK pathway (e.g., AZD6244, dabrafenib, LGX818, PD0325901, pimasertib, trametinib, or vemurafenib); an inhibitor of PI3K, mTOR, or Akt (e.g., as described herein); a CDK inhibitor (e.g., as described herein); a PKC inhibitor (e.g., LXS196 or sotrastaurin); an antibody-based therapeutic (e.g., an anti-PD-1 or anti-PDL1 antibody such as atezolizumab, pembrolizumab, nivolumab, or spartalizumab; an anti-CD20 antibody such as obinutuzumab or rituximab; or an anti-DR5 antibody such as drozitumab); a proteasome inhibitor (e.g., bortezomib, carfilzomib, ixazomib, or MG-132); an HDAC inhibitor (e.g., SAHA or VPA); an antibiotic (e.g., actinomycin D); a zinc-containing therapeutic (e.g., zinc or ZMC1); an HSP inhibitor (e.g., geldanamycin); an ATPase inhibitor (e.g., archazolid); a mitotic inhibitor (e.g., paclitaxel or vincristine); metformin; methotrexate; tanshinone IIA; and/or P5091.

In some embodiments, an anti-cancer therapy of the disclosure comprises a tyrosine kinase inhibitor, e.g., alone or in combination with an ABL1-targeted therapy. In some embodiments, the tyrosine kinase inhibitor is (a) a small molecule that inhibits one or more enzymatic activities of a tyrosine kinase, (b) an antibody that inhibits one or more activities of a tyrosine kinase (e.g., by binding to and inhibiting one or more activities of the tyrosine kinase, binding to and inhibiting expression, such as cell surface expression, of the tyrosine kinase, and/or binding to and inhibiting one or more activities of a cell expressing the tyrosine kinase, such as by inducing antibody-dependent cellular cytotoxicity, ADCC, or phagocytosis, ADCP), or (c) a nucleic acid that inhibits expression of a tyrosine kinase (e.g., an antisense oligonucleotide, miRNA, siRNA, morpholino, CRISPR-based therapeutic, and the like). In some embodiments, the tyrosine kinase inhibitor is a small molecule inhibitor of a tyrosine kinase (e.g., a competitive or non-competitive inhibitor). Non-limiting examples of tyrosine kinase inhibitors include imatinib, crenolanib, linifanib, ninetedanib, axitinib, dasatinib, imetelstat, midostaurin, pazopanib, sorafenib, sunitinb, motesanib, masitinib, vatalanib, cabozanitinib, tivozanib, OSI-930, Ki8751, telatinib, dovitinib, tyrphostin AG 1296, and amuvatinib, as well as pharmaceutically acceptable salts thereof.

In some embodiments, an anti-cancer therapy of the disclosure comprises a mitogen-activated protein kinase (MEK) inhibitor, e.g., alone or in combination with an ABL1-targeted therapy. In some embodiments, the MEK inhibitor inhibits one or more activities of MEK1 and/or MEK2. In some embodiments, the anti-cancer therapy/MEK inhibitor is (a) a small molecule that inhibits one or more enzymatic activities of MEK, (b) an antibody that inhibits one or more activities of MEK (e.g., by binding to and inhibiting one or more activities of MEK, binding to and inhibiting expression of MEK, and/or binding to and inhibiting one or more activities of a cell expressing MEK, such as by inducing antibody-dependent cellular cytotoxicity, ADCC, or phagocytosis, ADCP), or (c) a nucleic acid that inhibits expression of MEK (e.g., an antisense oligonucleotide, miRNA, siRNA, morpholino, CRISPR-based therapeutic, and the like). In some embodiments, the MEK inhibitor is a small molecule inhibitor of MEK (e.g., a competitive or non-competitive inhibitor). Non-limiting examples of MEK inhibitors include trametinib, cobimetinib, binimetinib, CI-1040, PD0325901, selumetinib, AZD8330, TAK-733, GDC-0623, refametinib, pimasertib, RO4987655, RO5126766, WX-544, and HL-085, as well as pharmaceutically acceptable salts thereof. In some embodiments, the anti-cancer therapy inhibits one or more activities of the Raf/MEK/ERK pathway, including inhibitors of Raf, MEK, and/or ERK.

In some embodiments, an anti-cancer therapy of the disclosure comprises a mammalian target of rapamycin (mTOR) inhibitor, e.g., alone or in combination with an ABL1-targeted therapy. In some embodiments, the mTOR inhibitor is (a) a small molecule that inhibits one or more enzymatic activities of mTOR, (b) an antibody that inhibits one or more activities of mTOR (e.g., by binding to and inhibiting one or more activities of mTOR, binding to and inhibiting expression of mTOR, and/or binding to and inhibiting one or more activities of a cell expressing mTOR, such as by inducing antibody-dependent cellular cytotoxicity, ADCC, or phagocytosis, ADCP), or (c) a nucleic acid that inhibits expression of mTOR (e.g., an antisense oligonucleotide, miRNA, siRNA, morpholino, CRISPR-based therapeutic, and the like). In some embodiments, the mTOR inhibitor is a small molecule inhibitor of mTOR (e.g., a competitive inhibitor, such as an ATP-competitive inhibitor, or a non-competitive inhibitor, such as a rapamycin analog). Non-limiting examples of mTOR inhibitors include temsirolimus, everolimus, ridaforolimus, dactolisib, GSK2126458, XL765, AZD8055, AZD2014, MLN128, PP242, NVP-BEZ235, LY3023414, PQR309, PKI587, and OSI027, as well as pharmaceutically acceptable salts thereof. In some embodiments, the anti-cancer therapy inhibits one or more activities of the Akt/mTOR pathway, including inhibitors of Akt and/or mTOR.

In some embodiments, an anti-cancer therapy of the disclosure comprises a PI3K inhibitor or Akt inhibitor, e.g., alone or in combination with an ABL1-targeted therapy. In some embodiments, the PI3K inhibitor inhibits one or more activities of PI3K. In some embodiments, the anti-cancer therapy/PI3K inhibitor is (a) a small molecule that inhibits one or more enzymatic activities of PI3K, (b) an antibody that inhibits one or more activities of PI3K (e.g., by binding to and inhibiting one or more activities of PI3K, binding to and inhibiting expression of PI3K, and/or binding to and inhibiting one or more activities of a cell expressing PI3K, such as by inducing antibody-dependent cellular cytotoxicity, ADCC, or phagocytosis, ADCP), or (c) a nucleic acid that inhibits expression of PI3K (e.g., an antisense oligonucleotide, miRNA, siRNA, morpholino, CRISPR-based therapeutic, and the like). In some embodiments, the PI3K inhibitor is a small molecule inhibitor of PI3K (e.g., a competitive or non-competitive inhibitor). Non-limiting examples of PI3K inhibitors include GSK2636771, buparlisib (BKM120), AZD8186, copanlisib (BAY80-6946), LY294002, PX-866, TGX115, TGX126, BEZ235, SF1126, idelalisib (GS-1101, CAL-101), pictilisib (GDC-094), GDC0032, IPI145, INK1117 (MLN1117), SAR260301, KIN-193 (AZD6482), duvelisib, GS-9820, GSK2636771, GDC-0980, AMG319, pazobanib, and alpelisib (BYL719, Piqray), as well as pharmaceutically acceptable salts thereof. In some embodiments, the AKT inhibitor inhibits one or more activities of AKT (e.g., AKT1). In some embodiments, the AKT inhibitor is (a) a small molecule that inhibits one or more enzymatic activities of AKT1, (b) an antibody that inhibits one or more activities of AKT1 (e.g., by binding to and inhibiting one or more activities of AKT1, binding to and inhibiting expression of AKT1, and/or binding to and inhibiting one or more activities of a cell expressing AKT1, such as by inducing antibody-dependent cellular cytotoxicity, ADCC, or phagocytosis, ADCP), or (c) a nucleic acid that inhibits expression of AKT1 (e.g., an antisense oligonucleotide, miRNA, siRNA, morpholino, CRISPR-based therapeutic, and the like). In some embodiments, the AKT1 inhibitor is a small molecule inhibitor of AKT1 (e.g., a competitive or non-competitive inhibitor). Non-limiting examples of AKT1 inhibitors include GSK690693, GSK2141795 (uprosertib), GSK2110183 (afuresertib), AZD5363, GDC-0068 (ipatasertib), AT7867, CCT128930, MK-2206, BAY 1125976, AKT1 and AKT2-IN-1, perifosine, and VIII, as well as pharmaceutically acceptable salts thereof. In some embodiments, the AKT1 inhibitor is a pan-Akt inhibitor.

In some embodiments, an anti-cancer therapy of the disclosure comprises a hedgehog (Hh) inhibitor, e.g., alone or in combination with an ABL1-targeted therapy. In some embodiments, the Hh inhibitor is (a) a small molecule that inhibits one or more enzymatic activities of Hh, (b) an antibody that inhibits one or more activities of Hh (e.g., by binding to and inhibiting one or more activities of Hh, binding to and inhibiting expression of Hh, and/or binding to and inhibiting one or more activities of a cell expressing Hh, such as by inducing antibody-dependent cellular cytotoxicity, ADCC, or phagocytosis, ADCP), or (c) a nucleic acid that inhibits expression of Hh (e.g., an antisense oligonucleotide, miRNA, siRNA, morpholino, CRISPR-based therapeutic, and the like). In some embodiments, the Hh inhibitor is a small molecule inhibitor of Hh (e.g., a competitive or non-competitive inhibitor). Non-limiting examples of Hh inhibitors include sonidegib, vismodegib, erismodegib, saridegib, BMS833923, PF-04449913, and LY2940680, as well as pharmaceutically acceptable salts thereof.

In some embodiments, an anti-cancer therapy of the disclosure comprises a heat shock protein (HSP) inhibitor, a MYC inhibitor, an HDAC inhibitor, an immunotherapy, a neoantigen, a vaccine, or a cellular therapy, e.g., alone or in combination with an ABL1-targeted therapy.

In some embodiments, the anti-cancer therapy comprises one or more of an immune checkpoint inhibitor, a chemotherapy, a VEGF inhibitor, an Integrin β3 inhibitor, a statin, an EGFR inhibitor, an mTOR inhibitor, a PI3K inhibitor, a MAPK inhibitor, or a CDK4/6 inhibitor, e.g., alone or in combination with an ABL1-targeted therapy.

In some embodiments, the anti-cancer therapy comprises a kinase inhibitor, e.g., alone or in combination with an ABL1-targeted therapy. In some embodiments, the kinase inhibitor is crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, or TAE684 (NVP-TAE684). In some embodiments, the kinase inhibitor is an ALK kinase inhibitor, e.g., as described in examples 3-39 of WO2005016894, which is incorporated herein by reference.

In some embodiments, the anti-cancer therapy comprises a heat shock protein (HSP) inhibitor, e.g., alone or in combination with an ABL1-targeted therapy. In some embodiments, the HSP inhibitor is a Pan-HSP inhibitor, such as KNK423. In some embodiments, the HSP inhibitor is an HSP70 inhibitor, such as cmHsp70.1, quercetin, VER155008, or 17-AAD. In some embodiments, the HSP inhibitor is a HSP90 inhibitor. In some embodiments, the HSP90 inhibitor is 17-AAD, Debio0932, ganetespib (STA-9090), retaspimycin hydrochloride (retaspimycin, IPI-504), AUY922, alvespimycin (KOS-1022, 17-DMAG), tanespimycin (KOS-953, 17-AAG), DS 2248, or AT13387 (onalespib). In some embodiments, the HSP inhibitor is an HSP27 inhibitor, such as Apatorsen (OGX-427).

In some embodiments, the anti-cancer therapy comprises a MYC inhibitor, e.g., alone or in combination with an ABL1-targeted therapy. In some embodiments, the MYC inhibitor is MYCi361 (NUCC-0196361), MYCi975 (NUCC-0200975), Omomyc (dominant negative peptide), ZINC16293153 (Min9), 10058-F4, JKY-2-169, 7594-0035, or inhibitors of MYC/MAX dimerization and/or MYC/MAX/DNA complex formation.

In some embodiments, the anti-cancer therapy comprises a histone deacetylase (HDAC) inhibitor, e.g., alone or in combination with an ABL1-targeted therapy. In some embodiments, the HDAC inhibitor is belinostat (PXD101, e.g., Beleodaq®), SAHA (vorinostat, suberoylanilide hydroxamine, e.g., Zolinza®), panobinostat (LBH589, LAQ-824), ACY1215 (Rocilinostat), quisinostat (JNJ-26481585), abexinostat (PCI-24781), pracinostat (SB939), givinostat (ITF2357), resminostat (4SC-201), trichostatin A (TSA), MS-275 (etinostat), Romidepsin (depsipeptide, FK228), MGCD0103 (mocetinostat), BML-210, CAY10603, valproic acid, MC1568, CUDC-907, CI-994 (Tacedinaline), Pivanex (AN-9), AR-42, Chidamide (CS055, HBI-8000), CUDC-101, CHR-3996, MPTOE028, BRD8430, MRLB-223, apicidin, RGFP966, BG45, PCI-34051, C149 (NCC149), TMP269, Cpd2, T247, T326, LMK235, C1A, HPOB, Nexturastat A, Befexamac, CBHA, Phenylbutyrate, MC1568, SNDX275, Scriptaid, Merck60, PX089344, PX105684, PX117735, PX117792, PX117245, PX105844, compound 12 as described by Li et al., Cold Spring Harb Perspect Med (2016) 6(10):a026831, or PX117445.

In some embodiments, the anti-cancer therapy comprises a VEGF inhibitor, e.g., alone or in combination with an ABL1-targeted therapy. In some embodiments, the VEGF inhibitor is Bevacizumab (e.g., Avastin®), BMS-690514, ramucirumab, pazopanib, sorafenib, sunitinib, golvatinib, vandetanib, cabozantinib, levantinib, axitinib, cediranib, tivozanib, lucitanib, semaxanib, nindentanib, regorafinib, or aflibercept.

In some embodiments, the anti-cancer therapy comprises an integrin β3 inhibitor, e.g., alone or in combination with an ABL1-targeted therapy. In some embodiments, the integrin β3 inhibitor is anti-avb3 (clone LM609), cilengitide (EMD121974, NSC, 707544), an siRNA, GLPG0187, MK-0429, CNTO95, TN-161, etaracizumab (MEDI-522), intetumumab (CNTO95) (anti-alphaV subunit antibody), abituzumab (EMD 525797/DI17E6) (anti-alphaV subunit antibody), JSM6427, SJ749, BCH-15046, SCH221153, or SC56631. In some embodiments, the anti-cancer therapy comprises an αIIbβ3 integrin inhibitor, e.g., alone or in combination with an ABL1-targeted therapy. In some embodiments, the alIbβ3 integrin inhibitor is abciximab, eptifibatide (e.g., Integrilin®), or tirofiban (e.g., Aggrastat®).

In some embodiments, the anti-cancer therapy comprises an mTOR inhibitor, e.g., alone or in combination with an ABL1-targeted therapy. In some embodiments, the mTOR inhibitor is temsirolimus (CCI-779), KU-006379, PP242, Torin1, Torin2, ICSN3250, Rapalink-1, CC-223, sirolimus (rapamycin), everolimus (RAD001), dactosilib (NVP-BEZ235), GSK2126458, WAY-001, WAY-600, WYE-687, WYE-354, SF1126, XL765, INK128 (MLN012), AZD8055, OSI027, AZD2014, or AP-23573.

In some embodiments, the anti-cancer therapy comprises a statin or a statin-based agent, e.g., alone or in combination with an ABL1-targeted therapy. In some embodiments, the statin or statin-based agent is simvastatin, atorvastatin, fluvastatin, pitavastatin, pravastatin, rosuvastatin, or cerivastatin.

In some embodiments, the anti-cancer therapy comprises a MAPK inhibitor, e.g., alone or in combination with an ABL1-targeted therapy. In some embodiments, the MAPK inhibitor is SB203580, SKF-86002, BIRB-796, SC-409, RJW-67657, BIRB-796, VX-745, RO3201195, SB-242235, or MW181.

In some embodiments, the anti-cancer therapy comprises an EGFR inhibitor, e.g., alone or in combination with an ABL1-targeted therapy. In some embodiments, the EGFR inhibitor is cetuximab, panitumumab, lapatinib, gefitinib, vandetanib, dacomitinib, icotinib, osimertinib (AZD9291), afatanib, olmutinib, EGF816 (nazartinib), avitinib (AC0010), rociletinib (CO-1686), BMS-690514, YH5448, PF-06747775, ASP8273, PF299804, AP26113, necitumumab (e.g., Portrazza®), or erlotinib. In some embodiments, the EGFR inhibitor is gefitinib or cetuximab.

In some embodiments, the anti-cancer therapy comprises a cancer immunotherapy, such as a checkpoint inhibitor, cancer vaccine, cell-based therapy, T cell receptor (TCR)-based therapy, adjuvant immunotherapy, cytokine immunotherapy, and oncolytic virus therapy, e.g., alone or in combination with an ABL1-targeted therapy. In some embodiments, the cancer immunotherapy comprises a small molecule, nucleic acid, polypeptide, carbohydrate, toxin, cell-based agent, or cell-binding agent. Examples of cancer immunotherapies are described in greater detail herein but are not intended to be limiting. In some embodiments, the cancer immunotherapy activates one or more aspects of the immune system to attack a cell (e.g., a tumor cell) that expresses a neoantigen, e.g., a neoantigen corresponding to an ABL1 fusion nucleic acid molecule or polypeptide of the disclosure. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine, e.g., alone or in combination with an ABL1-targeted therapy. A range of cancer vaccines have been tested that employ different approaches to promoting an immune response against a cancer (see, e.g., Emens L A, Expert Opin Emerg Drugs 13 (2): 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A. et al, J. Clin. Invest. 95:341, 1995), naked cDNA or peptides, encapsulated e.g., in poly(DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196:17-32, 1996). In some embodiments, a cancer vaccine is formulated as a peptide-based vaccine, or nucleic acid based vaccine in which the nucleic acid encodes the polypeptides. In some embodiments, a cancer vaccine is formulated as an antibody-based vaccine. In some embodiments, a cancer vaccine is formulated as a cell based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104:14-21, 2013). In some embodiments, such cancer vaccines augment the anti-cancer response.

In some embodiments, the cancer vaccine comprises a polynucleotide that encodes a neoantigen, e.g., a neoantigen corresponding to an ABL1 fusion nucleic acid molecule or polypeptide of the disclosure. In some embodiments, the cancer vaccine comprises DNA that encodes a neoantigen, e.g., a neoantigen corresponding to an ABL1 fusion nucleic acid molecule or polypeptide of the disclosure. In some embodiments, the cancer vaccine comprises RNA that encodes a neoantigen, e.g., a neoantigen corresponding to an ABL1 fusion nucleic acid molecule or polypeptide of the disclosure. In some embodiments, the cancer vaccine comprises a polynucleotide that encodes a neoantigen, e.g., a neoantigen corresponding to an ABL1 fusion nucleic acid molecule or polypeptide of the disclosure. In some embodiments, the cancer vaccine further comprises one or more additional antigens, neoantigens, or other sequences that promote antigen presentation and/or an immune response. In some embodiments, the polynucleotide is complexed with one or more additional agents, such as a liposome or lipoplex. In some embodiments, the polynucleotide(s) are taken up and translated by antigen presenting cells (APCs), which then present the neoantigen(s) via MHC class I on the APC cell surface.

In some embodiments, the cancer vaccine is selected from sipuleucel-T (e.g., Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (e.g., Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase-(TK-) deficient vaccinia virus engineered to express GM-CSF, for hepatocellular carcinoma (NCT02562755) and melanoma (NCT00429312); pelareorep (e.g., Reolysin®, Oncolytics Biotech), a variant of respiratory enteric orphan virus (reovirus) which does not replicate in cells that are not RAS-activated, in numerous cancers, including colorectal cancer (NCT01622543), prostate cancer (NCT01619813), head and neck squamous cell cancer (NCT01166542), pancreatic adenocarcinoma (NCT00998322), and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT 02759588); or CG0070 (Cold Genesys), an adenovirus engineered to express GM-CSF in bladder cancer (NCT02365818); anti-gp100; STINGVAX; GVAX; DCVaxL; and DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFa-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into an individual (possibly with pro-inflammatory or other attractants such as GM-CSF), taken up by cells in vivo to make the specific antigens, which then provoke the desired immune response. In some embodiments, vectors may be used to deliver more than one tumor antigen at a time, to increase the immune response. In addition, recombinant virus, bacteria or yeast vectors can trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12:771-776). In some embodiments, plasmid (or other vector) DNA that includes a sequence encoding a protein operably linked to regulatory elements required for gene expression is administered to individuals (e.g. human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a+-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some embodiments, the cancer immunotherapy comprises a cell-based therapy. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some embodiments, the immune response results in inhibition of tumor and/or metastatic cell growth and/or proliferation, and in related embodiments, results in neoplastic cell death and/or resorption. The immune cells can be derived from a different organism/host (exogenous immune cells) or can be cells obtained from the subject organism (autologous immune cells). In some embodiments, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g. chimeric). In some embodiments, a population of immune cells can be obtained from a subject in need of therapy or suffering from a disease associated with reduced immune cell activity. Thus, the cells will be autologous to the subject in need of therapy. In some embodiments, a population of immune cells can be obtained from a donor, such as a histocompatibility-matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible, in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some embodiments, the T cells are derived from the blood, bone marrow, lymph, umbilical cord, or lymphoid organs. In some embodiments, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs).

In some embodiments, the T cell-based therapy comprises a chimeric antigen receptor (CAR)-T cell-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen. In some embodiments, the CAR specifically binds a neoantigen, such as a neoantigen corresponding to an ABL1 fusion nucleic acid molecule or polypeptide of the disclosure.

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen. In some embodiments, the recombinant TCR specifically binds a neoantigen corresponding to an ABL1 fusion nucleic acid molecule or polypeptide of the disclosure.

In some embodiments, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize an antigen expressed by the tumor or cancer of the present disclosure. In some embodiments, the TILs are exposed to one or more neoantigens, e.g., a neoantigen corresponding to an ABL1 fusion nucleic acid molecule or polypeptide of the disclosure, e.g., a neoantigen, in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some embodiments, the cell-based therapy comprises a dendritic cell (DC)-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens to the patient's immune system. In some embodiments, the dendritic cells are autologous or allogeneic to the recipient.

In some embodiments, the cancer immunotherapy comprises a TCR-based therapy. In some embodiments, the cancer immunotherapy comprises administration of one or more TCRs or TCR-based therapeutics that specifically bind an antigen expressed by a cancer of the present disclosure, e.g., a neoantigen corresponding to an ABL1 fusion nucleic acid molecule or polypeptide of the disclosure. In some embodiments, the TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some embodiments, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the immunotherapy comprises cytokine immunotherapy. Cytokine immunotherapy comprises the use of one or more cytokines that activate components of the immune system. Examples include, but are not limited to, aldesleukin (e.g., PROLEUKIN®; interleukin-2), interferon alfa-2a (e.g., ROFERON®-A), interferon alfa-2b (e.g., INTRON®-A), and peginterferon alfa-2b (e.g., PEGINTRON®).

In some embodiments, the immunotherapy comprises oncolytic virus therapy. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g., from a “field source”) or modified replication-competent oncolytic virus. In some embodiments, the oncolytic virus, in addition to expressing a tumor antigen, may be modified to increase selectivity of the virus for cancer cells. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, a replicative oncolytic vaccinia virus expressing a tumor antigen may be engineered to lack one or more functional genes in order to increase the cancer selectivity of the virus. In some embodiments, an oncolytic vaccinia virus is engineered to lack thymidine kinase (TK) activity. In some embodiments, the oncolytic vaccinia virus may be engineered to lack vaccinia virus growth factor (VGF). In some embodiments, an oncolytic vaccinia virus may be engineered to lack both VGF and TK activity. In some embodiments, an oncolytic vaccinia virus may be engineered to lack one or more genes involved in evading host interferon (IFN) response such as E3L, K3L, B18R, or B8R. In some embodiments, a replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen may be locally or systemically administered to a subject, e.g. via intratumoral, intraperitoneal, intravenous, intra-arterial, intramuscular, intradermal, intracranial, subcutaneous, or intranasal administration.

In some embodiments, the anti-cancer therapy comprises an immune checkpoint inhibitor, e.g., alone or in combination with an ABL1-targeted therapy. In some embodiments, the methods provided herein comprise administering to an individual an effective amount of an immune checkpoint inhibitor. As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-H6, 2B4, ICOS, HVEM, CEACAM, LAIR1, CD80, CD86, CD276, VTCN1, MHC class I, MHC class II, GALS, adenosine, TGFR, CSFIR, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GAL9, VISTA (PD-1H), TIGIT, LAIR1, CD160, 2B4, TGFRbeta, A2AR, GITR (CD357), CD80 (B7-1), CD86 (B7-2), CD276 (B7-H3), VTCNI (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSF14, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (see, e.g., International Patent Publication WO2015016718; Pardoll, Nat Rev Cancer, 12 (4): 252-64, 2012; both incorporated herein by reference). In some embodiments, known inhibitors of immune checkpoint proteins or analogs thereof may be used, in particular chimerized, humanized or human forms of antibodies may be used.

In some embodiments, the checkpoint inhibitor is a PD-L1 axis binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCDI LG1,” “CD274,” “B7-H,” and “PDL1.” An exemplary human PD-L1 is shown in UniProtKB/Swiss-Prot Accession No.Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the PD-1 binding antagonist is a molecule that inhibits the binding of PD-1 to its ligand binding partners. In a specific embodiment, the PD-1 ligand binding partners are PD-L1 and/or PD-L2. In another instance, a PD-L1 binding antagonist is a molecule that inhibits the binding of PD-L1 to its binding ligands. In a specific embodiment, PD-L1 binding partners are PD-1 and/or B7-1. In another instance, the PD-L2 binding antagonist is a molecule that inhibits the binding of PD-L2 to its ligand binding partners. In a specific embodiment, the PD-L2 binding ligand partner is PD-1. The antagonist may be an antibody, an antigen binding fragment thereof, an immunoadhesin, a fusion protein, or an oligopeptide. In some embodiments, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is one or more of MDX-1 106 (nivolumab), MK-3475 (pembrolizumab, e.g., Keytruda®), MEDI-0680 (AMP-514), PDR001, REGN2810, MGA-012, JNJ-63723283, BI 754091, or BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist is AMP-224. Other examples of anti-PD-1 antibodies include, but are not limited to, MEDI-0680 (AMP-514; AstraZeneca), PDR001 (CAS Registry No. 1859072-53-9; Novartis), REGN2810 (e.g., LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANB011; Tesaro/AnaptysBio), AM0001 (ARMO Biosciences), ENUM 244C8 (Enumeral Biomedical Holdings), or ENUM 388D4 (Enumeral Biomedical Holdings). In some embodiments, the PD-1 axis binding antagonist comprises tislelizumab (BGB-A317), BGB-108, STI-A1110, AM0001, BI 754091, sintilimab (IBI308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, e.g., Keytruda®), PF-06801591, cemiplimab (REGN-2810, REGEN2810), dostarlimab (TSR-042, ANB011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, or CCX-4503, or derivatives thereof.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In some embodiments, the PD-L1 binding antagonist is an anti-PD-L1 antibody. In some embodiments, the anti-PD-L1 antibody can bind to a human PD-L1, for example a human PD-L1 as shown in UniProtKB/Swiss-Prot Accession No.Q9NZQ7.1, or a variant thereof. In some embodiments, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting the binding between PD-L1 and PD-1, and/or between PD-L1 and B7-1. In some instances, the anti-PD-L1 antibody is a monoclonal antibody. In some instances, the anti-PD-L1 antibody is an antibody fragment selected from a Fab, Fab′-SH, Fv, scFv, or (Fab′) 2 fragment. In some instances, the anti-PD-L1 antibody is a humanized antibody. In some instances, the anti-PD-L1 antibody is a human antibody. In some instances, the anti-PD-L1 antibody is selected from YW243.55.S70, MPDL3280A (atezolizumab), MDX-1 105, MEDI4736 (durvalumab), or MSB0010718C (avelumab). In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof.

In some embodiments, the checkpoint inhibitor is an antagonist of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), a carbohydrate, a lipid, a metal, or a toxin. In some embodiments, the CTLA-4 inhibitor comprises ipilimumab (IBI310, BMS-734016, MDX010, MDX-CTLA4, MEDI4736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the anti-PD-1 antibody or antibody fragment is MDX-1106 (nivolumab), MK-3475 (pembrolizumab, e.g., Keytruda®), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the PD-1 binding antagonist is an anti-PD-1 immunoadhesin. In some embodiments, the anti-PD-1 immunoadhesin is AMP-224. In some embodiments, the anti-PD-L1 antibody or antibody fragment is YW243.55.S70, MPDL3280A (atezolizumab), MDX-1105, MEDI4736 (durvalumab), MSB0010718C (avelumab), LY3300054, STI-A1014, KN035, FAZ053, or CX-072.

In some embodiments, the immune checkpoint inhibitor comprises a LAG-3 inhibitor (e.g., an antibody, an antibody conjugate, or an antigen-binding fragment thereof). In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some embodiments, the anti-cancer therapy comprises an immunoregulatory molecule or a cytokine, e.g., alone or in combination with an ABL1-targeted therapy. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, glp10, TCA-3, MCP-1, MIF, MIP-1α, MIP-1β, Rantes, macrophage colony stimulating factor (M-CSF), granulocyte colony stimulating factor (G-CSF), or granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3α (Lax), MIP-3β, Hcc-1, MPIF-1, MPIF-2, MCP-2, MCP-3, MCP-4, MCP-5, Eotaxin, Tarc, Elc, I309, IL-8, GCP-2 Groα, Gro-β, Nap-2, Ena-78, Ip-10, MIG, I-Tac, SDF-1, or BCA-1 (Blc), as well as functional fragments thereof. In some embodiments, the immunoregulatory molecule is included with any of the treatments provided herein.

In some embodiments, the immune checkpoint inhibitor is monovalent and/or monospecific. In some embodiments, the immune checkpoint inhibitor is multivalent and/or multispecific.

In some embodiments, the anti-cancer therapy comprises an anti-cancer agent that inhibits expression of a nucleic acid that comprises or encodes an ABL1 fusion nucleic acid molecule of the disclosure or a portion thereof, or an ABL1 fusion polypeptide of the disclosure, or a portion thereof. In some embodiments, the anti-cancer therapy comprises a nucleic acid molecule, such as a dsRNA, an siRNA, or an shRNA. As is known in the art, dsRNAs having a duplex structure are effective at inducing RNA interference (RNAi). In some embodiments, the anti-cancer therapy comprises a small interfering RNA molecule (siRNA). dsRNAs and siRNAs can be used to silence gene expression in mammalian cells (e.g., human cells). In some embodiments, a dsRNA of the disclosure comprises any of between about 5 and about 10 base pairs, between about 10 and about 12 base pairs, between about 12 and about 15 base pairs, between about 15 and about 20 base pairs, between about 20 and 23 base pairs, between about 23 and about 25 base pairs, between about 25 and about 27 base pairs, or between about 27 and about 30 base pairs. As is known in the art, siRNAs are small dsRNAs that optionally include overhangs. In some embodiments, the duplex region of an siRNA is between about 18 and 25 nucleotides, e.g., any of 18, 19, 20, 21, 22, 23, 24, or 25 nucleotides. siRNAs may also include short hairpin RNAs (shRNAs), e.g., with approximately 29-base-pair stems and 2-nucleotide 3′ overhangs. In some embodiments, a dsRNA, an siRNA, or an shRNA of the disclosure comprises a nucleotide sequence that is configured to hybridize to a nucleic acid that comprises or encodes a fusion nucleic acid molecule of the disclosure or a portion thereof comprising a breakpoint. Methods for designing, optimizing, producing, and using dsRNAs, siRNAs, or shRNAs, are known in the art.

In some embodiments, the anti-cancer therapy comprises a chemotherapy, e.g., alone or in combination with an ABL1-targeted therapy. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, famesyl-protein tansferase inhibitors, transplatinum, and pharmaceutically acceptable salts, acids, or derivatives of any of the above.

Some non-limiting examples of chemotherapeutic drugs which can be combined with anti-cancer therapies of the present disclosure are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

In some embodiments, the anti-cancer therapy comprises a kinase inhibitor, e.g., alone or in combination with an ABL1-targeted therapy. Examples of kinase inhibitors include those that target one or more receptor tyrosine kinases, e.g., BCR-ABL, B-Raf, EGFR, HER-2/ErbB2, IGF-IR, PDGFR-a, PDGFR-β, cKit, Flt-4, Flt3, FGFR1, FGFR2, FGFR3, FGFR4, CSFIR, c-Met, ROS1, RON, c-Ret, or ALK; one or more cytoplasmic tyrosine kinases, e.g., c-SRC, c-YES, Abl, or JAK-2; one or more serine/threonine kinases, e.g., ATM, Aurora A & B, CDKs, mTOR, PKCi, PLKs, b-Raf, c-Raf, S6K, or STK11/LKB1; or one or more lipid kinases, e.g., PI3K or SKI. Small molecule kinase inhibitors include PHA-739358, nilotinib, dasatinib, PD166326, NSC 743411, lapatinib (GW-572016), canertinib (CI-1033), semaxinib (SU5416), vatalanib (PTK787/ZK222584), sutent (SU1 1248), sorafenib (BAY 43-9006), or leflunomide (SU101). Additional non-limiting examples of tyrosine kinase inhibitors include imatinib (Gleevec/Glivec) and gefitinib (Iressa).

In some embodiments, the anti-cancer therapy comprises any of abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof, e.g., alone or in combination with an ABL1-targeted therapy.

In some embodiments, the anti-cancer therapy comprises an anti-angiogenic agent, e.g., alone or in combination with an ABL1-targeted therapy. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-β and IFN-γ, CXCL10, IL-4, IL-12 and IL-18, prothrombin (kringle domain-2), antithrombin III fragment, prolactin, VEGI, SPARC, osteopontin, maspin, canstatin, proliferin-related protein, restin and drugs such as bevacizumab, itraconazole, carboxyamidotriazole, TNP-470, CM101, IFN-α platelet factor-4, suramin, SU5416, thrombospondin, VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina v β3 inhibitors, linomide, or tasquinimod. In some embodiments, known therapeutic candidates that may be used according to the methods of the disclosure include naturally occurring angiogenic inhibitors, including without limitation, angiostatin, endostatin, or platelet factor-4. In another embodiment, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF, VEGF or PDGF. In some embodiments, anti-angiogenic agents that may be used according to the methods of the disclosure include, without limitation, suramin and its analogs, and tecogalan. In other embodiments, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used according to the methods of the disclosure is an antibody to VEGF, such as Avastin®/bevacizumab (Genentech).

In some embodiments, the anti-cancer therapy comprises an anti-DNA repair therapy, e.g., alone or in combination with an ABL1-targeted therapy. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some embodiments, the anti-cancer therapy comprises a radiosensitizer, e.g., alone or in combination with an ABL1-targeted therapy. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some embodiments, the anti-cancer therapy comprises an anti-inflammatory agent, e.g., alone or in combination with an ABL1-targeted therapy. In some embodiments, the anti-inflammatory agent is an agent that blocks, inhibits, or reduces inflammation or signaling from an inflammatory signaling pathway In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNβ, IFNγ, IFN-γ inducing factor (IGIF); transforming growth factor-β (TGF-β); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-κB; TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (e.g., Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (e.g., ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade®), golimumab (Simponi®), adalimumab (e.g., Humira®), certolizumab pegol (e.g., Cimzia®) or etanercept. In some embodiments, the anti-inflammatory agent is a corticosteroid. Exemplary corticosteroids include, but are not limited to, cortisone (hydrocortisone, hydrocortisone sodium phosphate, hydrocortisone sodium succinate, e.g., Ala-Cort®, Hydrocort Acetate®, hydrocortone phosphate Lanacort®, Solu-Cortef®), decadron (dexamethasone, dexamethasone acetate, dexamethasone sodium phosphate, e.g., Dexasone®, Diodex®, Hexadrol®, Maxidex®), methylprednisolone (6-methylprednisolone, methylprednisolone acetate, methylprednisolone sodium succinate, e.g., Duralone®, Medralone®, Medrol®, M-Prednisol®, Solu-Medrol®), prednisolone (e.g., Delta-Cortef®, ORAPRED®, Pediapred®, Prezone®), and prednisone (e.g., Deltasone®, Liquid Pred®, Meticorten®, Orasone®), and bisphosphonates (e.g., pamidronate (Aredia®), and zoledronic acid (e.g., Zometac®).

In some embodiments, the anti-cancer therapy comprises an anti-hormonal agent, e.g., alone or in combination with an ABL1-targeted therapy. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGACE® megestrol acetate, AROMASIN® exemestane, formestanie, fadrozole, RIVISOR® vorozole, FEMARA® letrozole, and ARIMIDEX® (anastrozole); anti-androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In some embodiments, the anti-cancer therapy comprises an antimetabolite chemotherapeutic agent, e.g., alone or in combination with an ABL1-targeted therapy. Antimetabolite chemotherapeutic agents are agents that are structurally similar to a metabolite, but cannot be used by the body in a productive manner. Many antimetabolite chemotherapeutic agents interfere with the production of RNA or DNA. Examples of antimetabolite chemotherapeutic agents include gemcitabine (e.g., GEMZAR®), 5-fluorouracil (5-FU), capecitabine (e.g., XELODA™), 6-mercaptopurine, methotrexate, 6-thioguanine, pemetrexed, raltitrexed, arabinosylcytosine ARA-C cytarabine (e.g., CYTOSAR-U®), dacarbazine (DTIC-DOMED), azocytosine, deoxycytosine, pyridmidene, fludarabine (e.g., FLUDARA®), cladrabine, and 2-deoxy-D-glucose. In some embodiments, an antimetabolite chemotherapeutic agent is gemcitabine. Gemcitabine HCL is sold by Eli Lilly under the trademark GEMZAR®.

In some embodiments, the anti-cancer therapy comprises a platinum-based chemotherapeutic agent, e.g., alone or in combination with an ABL1-targeted therapy. Platinum-based chemotherapeutic agents are chemotherapeutic agents that comprise an organic compound containing platinum as an integral part of the molecule. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

In some aspects, provided herein are therapeutic formulations comprising an anti-cancer therapy provided herein, and a pharmaceutically acceptable carrier, excipient, or stabilizer. A formulation provided herein may contain more than one active compound, e.g., an anti-cancer therapy provided herein and one or more additional agents (e.g., anti-cancer agents).

Acceptable carriers, excipients, or stabilizers are non-toxic to recipients at the dosages and concentrations employed, and include, for example, one or more of: buffers such as phosphate, citrate, and other organic acids; antioxidants, including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, or m-cresol; low molecular weight polypeptides (e.g., less than about 10 residues); proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); surfactants such as non-ionic surfactants; or polymers such as polyethylene glycol (PEG).

The active ingredients may be entrapped in microcapsules. Such microcapsules may be prepared, for example, by coacervation techniques or by interfacial polymerization, for example, hydroxymethylcellulose or gelatin-microcapsules and poly-(methylmethacylate) microcapsules, respectively; in colloidal drug delivery systems (for example, liposomes, albumin microspheres, microemulsions, nano-particles and nano-capsules); or in macroemulsions. Such techniques are known in the art.

Sustained-release compositions may be prepared. Suitable examples of sustained-release compositions include semi-permeable matrices of solid hydrophobic polymers containing an anti-cancer therapy of the disclosure. Such matrices may be in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

A formulation provided herein may also contain more than one active compound, for example, those with complementary activities that do not adversely affect each other. The type and effective amounts of such medicaments depend, for example, on the amount and type of active compound(s) present in the formulation, and clinical parameters of the subjects.

For general information concerning formulations, see, e.g., Gilman et al. (eds.) The Pharmacological Bases of Therapeutics, 8th Ed., Pergamon Press, 1990; A. Gennaro (ed.), Remington's Pharmaceutical Sciences, 18th Edition, Mack Publishing Co., Pennsylvania, 1990; Avis et al. (eds.) Pharmaceutical Dosage Forms: Parenteral Medications Dekker, New York, 1993; Lieberman et al. (eds.) Pharmaceutical Dosage Forms: Tablets Dekker, New York, 1990; Lieberman et al. (eds.), Pharmaceutical Dosage Forms: Disperse Systems Dekker, New York, 1990; and Walters (ed.) Dermatological and Transdermal Formulations (Drugs and the Pharmaceutical Sciences), Vol 1 19, Marcel Dekker, 2002.

Formulations to be used for in vivo administration are sterile. This is readily accomplished by filtration through sterile filtration membranes or other methods known in the art.

In some embodiments, an anti-cancer therapy of the disclosure is administered as a monotherapy. In some embodiments, the anti-cancer therapy is administered in combination with one or more additional anti-cancer therapies or treatments, e.g., as described herein. In some embodiments, the one or more additional anti-cancer therapies or treatments include one or more anti-cancer therapies described herein. In some embodiments, the methods of the present disclosure comprise administration of any combination of any of the anti-cancer therapies provided herein. In some embodiments, the additional anti-cancer therapy comprises one or more of surgery, radiotherapy, chemotherapy, anti-angiogenic therapy, anti-DNA repair therapy, and anti-inflammatory therapy. In some embodiments, the additional anti-cancer therapy comprises an anti-neoplastic agent, a chemotherapeutic agent, a growth inhibitory agent, an anti-angiogenic agent, a radiation therapy, a cytotoxic agent, or combinations thereof. In some embodiments, an anti-cancer therapy may be administered in conjunction with a chemotherapy or chemotherapeutic agent. In some embodiments, the chemotherapy or chemotherapeutic agent is a platinum-based agent (including, without limitation cisplatin, carboplatin, oxaliplatin, and staraplatin). In some embodiments, an anti-cancer therapy may be administered in conjunction with a radiation therapy.

### D. Reporting

In some embodiments, the methods provided herein comprise generating a report, and/or providing a report to party.

In some embodiments, a report according to the present disclosure comprises information about one or more of: an ABL1 fusion nucleic acid molecule or ABL1 fusion polypeptide of the disclosure; a cancer of the disclosure, e.g., comprising an ABL1 fusion nucleic acid molecule or ABL1 fusion polypeptide of the disclosure; or a treatment, a therapy, or one or more treatment options for an individual having a cancer, such as a cancer of the disclosure (e.g., comprising an ABL1 fusion nucleic acid molecule or an ABL1 fusion polypeptide described herein).

In some embodiments, a report according to the present disclosure comprises information about the presence or absence of an ABL1 fusion nucleic acid molecule or an ABL1 fusion polypeptide of the disclosure in a sample obtained from an individual, such as an individual having a cancer, e.g., a cancer provided herein. In one embodiment, a report according to the present disclosure indicates that an ABL1 fusion nucleic acid molecule or an ABL1 fusion polypeptide of the disclosure is present in a sample obtained from the individual. In one embodiment, a report according to the present disclosure indicates that an ABL1 fusion nucleic acid molecule or an ABL1 fusion polypeptide of the disclosure is not present in a sample obtained from the individual. In one embodiment, a report according to the present disclosure indicates that an ABL1 fusion nucleic acid molecule or an ABL1 fusion polypeptide of the disclosure has been detected in a sample obtained from the individual. In one embodiment, a report according to the present disclosure indicates that an ABL1 fusion nucleic acid molecule or an ABL1 fusion polypeptide of the disclosure has not been detected in a sample obtained from the individual. In some embodiments, the report comprises an identifier for the individual from which the sample was obtained.

In some embodiments, the report includes information on the role of an ABL1 fusion nucleic acid molecule or an ABL1 fusion polypeptide of the disclosure, or its wild type counterparts, in disease, such as in cancer. Such information can include one or more of: information on prognosis of a cancer, such as a cancer provided herein, e.g., comprising an ABL1 fusion nucleic acid molecule or an ABL1 fusion polypeptide described herein; information on resistance of a cancer, such as a cancer provided herein, e.g., comprising an ABL1 fusion nucleic acid molecule or an ABL1 fusion polypeptide described herein, to one or more treatments; information on potential or suggested therapeutic options (e.g., such as an anti-cancer therapy provided herein, or a treatment selected or identified according to the methods provided herein); or information on therapeutic options that should be avoided. In some embodiments, the report includes information on the likely effectiveness, acceptability, and/or advisability of applying a therapeutic option (e.g., such as an anti-cancer therapy provided herein, or a treatment selected or identified according to the methods provided herein) to an individual having a cancer, such as a cancer provided herein, e.g., comprising an ABL1 fusion nucleic acid molecule or an ABL1 fusion polypeptide described herein and identified in the report. In some embodiments, the report includes information or a recommendation on the administration of a treatment (e.g., an anti-cancer therapy provided herein, or a treatment selected or identified according to the methods provided herein). In some embodiments, the information or recommendation includes the dosage of the treatment and/or a treatment regimen (e.g., in combination with other treatments, such as a second therapeutic agent). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: obtaining a sample, such as a sample described herein, from an individual, e.g., an individual having a cancer, such as a cancer provided herein; detecting an ABL1 fusion nucleic acid molecule or an ABL1 fusion polypeptide of the disclosure in the sample, or acquiring knowledge of the presence of the ABL1 fusion nucleic acid molecule or ABL1 fusion polypeptide of the disclosure in the sample; and generating a report. In some embodiments, a report generated according to the methods provided herein comprises one or more of: information about the presence or absence of an ABL1 fusion nucleic acid molecule or an ABL1 fusion polypeptide of the disclosure in the sample; an identifier for the individual from which the sample was obtained; information on the role of the ABL1 fusion nucleic acid molecule or ABL1 fusion polypeptide of the disclosure, or its wild type counterparts, in disease (e.g., such as in cancer); information on prognosis, resistance, or potential or suggested therapeutic options (such as an anti-cancer therapy provided herein, or a treatment selected or identified according to the methods provided herein); information on the likely effectiveness, acceptability, or the advisability of applying a therapeutic option (such as an anti-cancer therapy provided herein, or a treatment selected or identified according to the methods provided herein) to the individual; a recommendation or information on the administration of a treatment (such as an anti-cancer therapy provided herein, or a treatment selected or identified according to the methods provided herein); or a recommendation or information on the dosage or treatment regimen of a treatment (such as an anti-cancer therapy provided herein, or a treatment selected or identified according to the methods provided herein), e.g., in combination with other treatments (e.g., a second therapeutic agent). In some embodiments, the report generated is a personalized cancer report.

A report according to the present disclosure may be in an electronic, web-based, or paper form. The report may be provided to an individual or a patient (e.g., an individual or a patient with a cancer, such as a cancer provided herein, e.g., comprising an ABL1 fusion nucleic acid molecule or an ABL1 fusion polypeptide of the disclosure), or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting an ABL1 fusion nucleic acid molecule or an ABL1 fusion polypeptide of the disclosure in a sample obtained from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of an ABL1 fusion nucleic acid molecule or an ABL1 fusion polypeptide of the disclosure in a sample obtained from an individual (e.g., an individual having a cancer). In some instances, all or a portion of the report may be displayed in a graphical user interface of an online or web-based healthcare portal.

### E. Software, Systems, and Devices

In some other aspects, provided herein are non-transitory computer-readable storage media. In some embodiments, the non-transitory computer-readable storage media comprise one or more programs for execution by one or more processors of a device, the one or more programs including instructions which, when executed by the one or more processors, cause the device to perform A method according to any of the embodiments described herein.

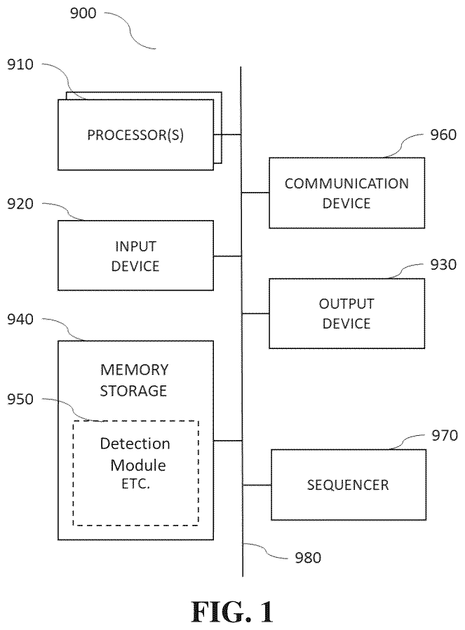

FIG. 1 illustrates an example of a computing device or system in accordance with one embodiment. Device 900 can be a host computer connected to a network. Device 900 can be a client computer or a server. As shown in FIG. 1, device 900 can be any suitable type of microprocessor-based device, such as a personal computer, workstation, server or handheld computing device (portable electronic device) such as a phone or tablet. The device can include, for example, one or more processor(s) 910, input devices 920, output devices 930, memory or storage devices 940, communication devices 960, and nucleic acid sequencers 970. Software 950 residing in memory or storage device 940 may comprise, e.g., an operating system as well as software for executing the methods described herein, e.g., for detecting an ABL1 fusion nucleic acid molecule of the disclosure. Input device 920 and output device 930 can generally correspond to those described herein, and can either be connectable or integrated with the computer.

Input device 920 can be any suitable device that provides input, such as a touch screen, keyboard or keypad, mouse, or voice-recognition device. Output device 930 can be any suitable device that provides output, such as a touch screen, haptics device, or speaker.

Storage 940 can be any suitable device that provides storage (e.g., an electrical, magnetic or optical memory including a RAM (volatile and non-volatile), cache, hard drive, or removable storage disk). Communication device 960 can include any suitable device capable of transmitting and receiving signals over a network, such as a network interface chip or device. The components of the computer can be connected in any suitable manner, such as via a wired media (e.g., a physical system bus 980, Ethernet connection, or any other wire transfer technology) or wirelessly (e.g., Bluetooth®, Wi-Fi®, or any other wireless technology).

Software module 950, which can be stored as executable instructions in storage 940 and executed by processor(s) 910, can include, for example, an operating system and/or the processes that embody the functionality of the methods of the present disclosure, e.g., for detecting an ABL1 fusion nucleic acid molecule of the disclosure (e.g., as embodied in the devices as described herein).

Software module 950 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 940, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 950 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

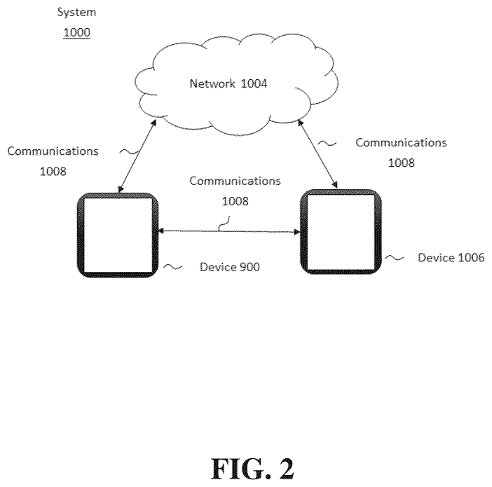

Device 900 may be connected to a network (e.g., network 1004, as shown in FIG. 2 and described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 900 can be implemented using any operating system, e.g., an operating system suitable for operating on the network. Software module 950 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 910.

Device 900 can further include a sequencer 970, which can be any suitable nucleic acid sequencing instrument. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio RS system.

FIG. 2 illustrates an example of a computing system in accordance with one embodiment. In computing system 1000, device 900 (e.g., as described above and illustrated in FIG. 1) is connected to network 1004, which is also connected to device 1006. In some embodiments, device 1006 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio RS system.

Devices 900 and 1006 may communicate, e.g., using suitable communication interfaces via network 1004, such as a Local Area Network (LAN), Virtual Private Network (VPN), or the Internet. In some embodiments, network 1004 can be, for example, the Internet, an intranet, a virtual private network, a cloud network, a wired network, or a wireless network. Devices 900 and 1006 may communicate, in part or in whole, via wireless or hardwired communications, such as Ethernet, IEEE 802.11b wireless, or the like. Additionally, devices 900 and 1006 may communicate, e.g., using suitable communication interfaces, via a second network, such as a mobile/cellular network.

Communication between devices 900 and 1006 may further include or communicate with various servers such as a mail server, mobile server, media server, telephone server, and the like. In some embodiments, devices 900 and 1006 can communicate directly (instead of, or in addition to, communicating via network 1004), e.g., via wireless or hardwired communications, such as Ethernet, IEEE 802.11b wireless, or the like. In some embodiments, devices 900 and 1006 communicate via communications 1008, which can be a direct connection or can occur via a network (e.g., network 1004).

One or all of devices 900 and 1006 generally include logic (e.g., http web server logic) or are programmed to format data, accessed from local or remote databases or other sources of data and content, for providing and/or receiving information via network 1004 according to various examples described herein.

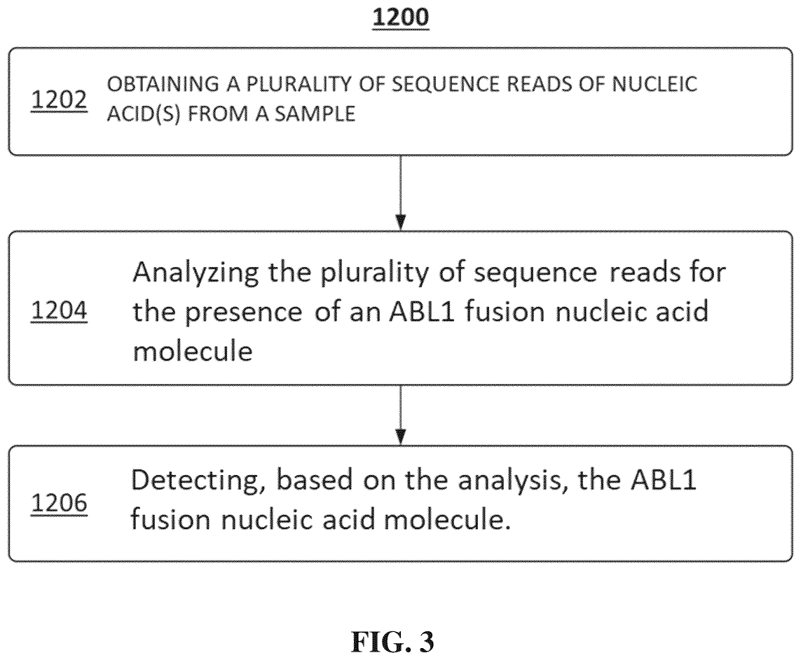

FIG. 3 illustrates an exemplary process 1200 for detecting an ABL1 fusion nucleic acid molecule of the disclosure in a sample, in accordance with some embodiments of the present disclosure. Process 1200 is performed, for example, using one or more electronic devices implementing a software program. In some examples, process 1200 is performed using a client-server system, and the blocks of process 1200 are divided up in any manner between the server and a client device. In other examples, the blocks of process 1200 are divided up between the server and multiple client devices. Thus, while portions of process 1200 are described herein as being performed by particular devices of a client-server system, it will be appreciated that process 1200 is not so limited. In some embodiments, the executed steps can be executed across many systems, e.g., in a cloud environment. In other examples, process 1200 is performed using only a client device or only multiple client devices. In process 1200, some blocks are, optionally, combined, the order of some blocks is, optionally, changed, and some blocks are, optionally, omitted. In some examples, additional steps may be performed in combination with the process 1200. Accordingly, the operations as illustrated (and described in greater detail below) are exemplary by nature and, as such, should not be viewed as limiting.

At block 1202, a plurality of sequence reads of one or more nucleic acid molecules is obtained, wherein the one or more nucleic acid molecules are derived from a sample obtained from an individual, e.g., as described herein. In some embodiments, the sample is obtained from an individual having a cancer, such as a cancer described herein. In some embodiments, the sequence reads are obtained using a sequencer, e.g., as described herein or otherwise known in the art. In some embodiments, the nucleic acid molecules comprise one or more nucleic acid molecules corresponding to: an ABL1 fusion nucleic acid molecule of the disclosure; or a gene involved in an ABL1 fusion nucleic acid molecule of the disclosure; or fragments thereof. Optionally, prior to obtaining the sequence reads, the sample is purified, enriched (e.g., for nucleic acid(s) corresponding to: an ABL1 fusion nucleic acid molecule of the disclosure; or a gene involved in an ABL1 fusion nucleic acid molecule of the disclosure; or fragments thereof), and/or subjected to PCR amplification. At block 1204, an exemplary system (e.g., one or more electronic devices) analyzes the plurality of sequence reads for the presence of an ABL1 fusion nucleic acid molecule of the disclosure, or a fragment thereof. At block 1206, the system detects (e.g., based on the analysis) an ABL1 fusion nucleic acid molecule of the disclosure, or a fragment thereof, in the sample.

In some embodiments of any of the methods, systems, devices, non-transitory computer readable storage media, or processes of the disclosure, the ABL1 fusion nucleic acid molecule is any of the ABL1 fusion nucleic acid molecules described herein (e.g., as described above, for example, in Section A herein; and/or in Example 1 herein). In some embodiments of any of the methods, systems, devices, non-transitory computer readable storage media, or processes of the disclosure, the ABL1 fusion polypeptide is any of the ABL1 fusion polypeptides described herein (e.g., as described above, for example, in Section A herein; and/or in Example 1 herein). In some embodiments, the cancer is any cancer known in the art or described herein (e.g., as described above, for example, in Section A herein; and/or in Example 1 herein). In some embodiments, any of the cancers described herein (e.g., as described above, for example, in Section A herein; and/or in Example 1 herein) may comprise any of the ABL1 fusion nucleic acid molecules or polypeptides of the disclosure (e.g., as described above, for example, in Section A herein; and/or in Example 1 herein). In some embodiments, a cancer provided in Table 2 in Example 1 herein comprises the corresponding ABL1 fusion nucleic acid molecule and/or ABL1 fusion polypeptide as described in Table 2.

In some embodiments of any of the methods, systems, devices, non-transitory computer readable storage media, or processes of the disclosure, detection of an ABL1 fusion nucleic acid molecule or polypeptide of the disclosure, in a cancer (e.g., in one or more samples) identifies the individual having the cancer as one who may benefit from a treatment comprising an anti-cancer therapy, e.g., an anti-cancer therapy provided herein, such as an ABL1-targeted therapy. In some embodiments of any of the methods, systems, devices, non-transitory computer readable storage media, or processes of the disclosure, detection of an ABL1 fusion nucleic acid molecule or polypeptide of the disclosure in a cancer (e.g., in one or more samples) predicts the individual having the cancer to have longer survival when treated with a treatment comprising an anti-cancer therapy, e.g., an ABL1-targeted therapy, as compared to survival of an individual whose cancer does not comprise an ABL1 fusion nucleic acid molecule or polypeptide. In some embodiments of any of the methods, systems, devices, non-transitory computer readable storage media, or processes of the disclosure, detection of an ABL1 fusion nucleic acid molecule or polypeptide of the disclosure in a cancer (e.g., in one or more samples) identifies the individual having the cancer to be a candidate to receive a treatment comprising an anti-cancer therapy, e.g., an ABL1-targeted therapy. In some embodiments of any of the methods, systems, devices, non-transitory computer readable storage media, or processes of the disclosure, detection of an ABL1 fusion nucleic acid molecule or polypeptide of the disclosure in a cancer (e.g., in one or more samples) identifies the individual having the cancer as likely to respond (e.g., to have a therapeutic response) to a treatment comprising an anti-cancer therapy, e.g., an ABL1-targeted therapy. In some embodiments of any of the methods, systems, devices, non-transitory computer readable storage media, or processes of the disclosure, detection of an ABL1 fusion nucleic acid molecule or polypeptide of the disclosure in a cancer (e.g., in one or more samples) identifies the individual having the cancer as likely to have an improved response when treated with a treatment comprising an anti-cancer therapy, e.g., an ABL1-targeted therapy, as compared to an individual whose cancer does not comprise an ABL1 fusion nucleic acid molecule or polypeptide.

In some embodiments of any of the methods, systems, devices, non-transitory computer readable storage media, or processes of the disclosure, the plurality of sequence reads is obtained by sequencing nucleic acids obtained from any of the samples described herein, e.g., tissue and/or liquid biopsies, etc. In some embodiments, the sample is obtained from the cancer. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, cerebrospinal fluid, sputum, stool, urine, or saliva. In some embodiments, the sample comprises cells and/or nucleic acids from the cancer. In some embodiments, the sample comprises mRNA, DNA, circulating tumor DNA (ctDNA), cell-free DNA, or cell-free RNA from the cancer. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

In some embodiments of any of the methods, systems, devices, non-transitory computer readable storage media, or processes of the disclosure, the plurality of sequence reads is obtained by sequencing. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the massively parallel sequencing technique comprises next generation sequencing (NGS).

In some embodiments of any of the methods, systems, devices, non-transitory computer readable storage media, or processes of the disclosure, the individual is administered a treatment based at least in part on detection of an ABL1 fusion nucleic acid molecule or polypeptide of the disclosure in the cancer in an individual (e.g., in one or more samples from the individual). In some embodiments, the treatment is an anti-cancer therapy known in the art or described herein, e.g., an ABL1-targeted therapy.

In some embodiments of any of the methods, systems, devices, non-transitory computer readable storage media, or processes of the disclosure, the disclosed methods for determining the presence or absence of an ABL1 fusion nucleic acid molecule of the disclosure may be implemented as part of a genomic profiling process that comprises identification of the presence of variant sequences at one or more gene loci in a sample derived from an individual as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods for determining the presence or absence of an ABL1 fusion nucleic acid molecule of the disclosure as part of a genomic profiling process can improve the validity of, e.g., disease detection calls, made on the basis of the genomic profiling by, for example, independently confirming the presence of the ABL1 fusion nucleic acid molecule of the disclosure in a given patient sample. In some embodiments, the one or more gene loci comprise the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKNIA, CDKNIB, CDKN2A, CDKN2B, CDKN2C, CEBPA, CHEK1, CHEK2, CIC, CREBBP, CRKL, CSFIR, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCA1P, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene locus, or any combination thereof. In some embodiments, the one or more gene loci comprise the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene locus, or any combination thereof.

In some instances, the comprehensive genomic profiling may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, the comprehensive genomic profiling may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some embodiments of any of the methods, systems, devices, non-transitory computer readable storage media, or processes of the disclosure, a molecular profile for a sample or for an individual is generated based at least in part on detecting an ABL1 fusion nucleic acid molecule of the disclosure, or a fragment thereof, in a sample. In some instances, the molecular profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors. In some instances, the molecular profile may comprise results from a comprehensive genomic profiling (CGP) test (e.g., as describe above), a nucleic acid sequencing-based test, a gene expression profiling test, a cancer hotspot panel test, a DNA methylation test, a DNA fragmentation test, an RNA fragmentation test, or any combination thereof. In some embodiments, the molecular profile further comprises/indicates/comprises information on presence or absence of mutations in one or more additional genes, e.g., a panel of known/suspected oncogenes and/or tumor suppressors. In some embodiments, the molecular profile is obtained from a genomic profiling assay (such as a cancer- or tumor-related genomic profiling assay), e.g., as obtained using any of the sequencing methodologies described herein. In some embodiments, the molecular profile includes information from whole-genome or whole-exome sequencing. In some embodiments, the molecular profile includes information from targeted sequencing. In some embodiments, the molecular profile includes information from NGS. In some embodiments, the molecular profile comprises/indicates/comprises information on presence or absence of mutations such as short variant alterations (e.g., a base substitution, insertion, or deletion), copy-number alterations (e.g., an amplification or a homozygous deletion), and/or rearrangements (e.g., a gene fusion or other genomic or chromosomal rearrangement) of one or more genes, e.g., a panel of known/suspected oncogenes and/or tumor suppressors, one or more cancer-related genes, or any combination thereof. In some embodiments, the individual is administered a treatment based at least in part on the molecular profile. In some embodiments, the treatment is an anti-cancer therapy known in the art or described herein. In some embodiments, the one or more genes comprise the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKNIA, CDKN1B, CDKN2A, CDKN2B, CDKN2C, CEBPA, CHEK1, CHEK2, CIC, CREBBP, CRKL, CSFIR, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKARIA, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCA1P, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene, or any combination thereof. In some embodiments, the one or more genes comprise the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene, or any combination thereof.

In some embodiments of any of the methods, systems, devices, non-transitory computer readable storage media, or processes of the disclosure, a report is generated, e.g., as described in further detail above. In some embodiments, the report comprises/indicates/comprises information on the presence or absence of an ABL1 fusion nucleic acid molecule or polypeptide of the disclosure in the cancer in an individual (e.g., in one or more samples from the individual). In some embodiments, the report comprises/indicates/comprises information on results of a genomic profiling process of a cancer in an individual (e.g., in one or more samples from the individual), e.g., as described above. In some embodiments, the report comprises/indicates/comprises information on results of comprehensive genomic profiling of a cancer in an individual (e.g., in one or more samples from the individual), e.g., as described above. In some embodiments, the report comprises/indicates/comprises information on a molecular profile generated for the individual or the sample, e.g., as described above. In some embodiments, the report comprises/indicates/comprises information on a treatment or one or more treatment options selected or identified for the individual, based, at least in part, on the presence of an ABL1 fusion nucleic acid molecule or polypeptide of the disclosure in the cancer in an individual (e.g., in one or more samples from the individual), and optionally based on results of a genomic profiling process, comprehensive genomic profiling, and/or a molecular profile generated for the individual or a sample, e.g., as described above. In some embodiments, the treatment or one or more treatment options comprise an anti-cancer therapy known in the art or described herein, e.g., an ABL1-targeted therapy. In some embodiments, the report is provided or transmitted to the individual, a caregiver, a healthcare provider, a physician, an oncologist, an electronic medical record system, a hospital, a clinic, a third-party payer, an insurance company, or a government office, e.g., as described in further detail above. In some embodiments, the report is transmitted via a computer network or a peer-to-peer connection. In some embodiments, an individual is administered a treatment based, at least in part, on the report. In some instances, all or a portion of the report may be displayed in a graphical user interface of an online or web-based healthcare portal.

The method steps of the methods described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

### IV. Articles of Manufacture or Kits

Provided herein are kits or articles of manufacture comprising one or more reagents for detecting an ABL1 fusion nucleic acid molecule of the disclosure in a sample; or an ABL1 fusion polypeptide encoded by an ABL1 fusion nucleic acid molecule of the disclosure.

In some embodiments, the kits or articles of manufacture comprise one or more probes of the disclosure for detecting an ABL1 fusion nucleic acid molecule of the disclosure in a sample, e.g., according to any detection method known in the art or described herein. In some embodiments, the kits or articles of manufacture comprise one or more baits (e.g., one or more bait molecules) of the disclosure for detecting an ABL1 fusion nucleic acid molecule of the disclosure in a sample, e.g., according to any detection method known in the art or described herein. In some embodiments, the kits or articles of manufacture comprise one or more oligonucleotides (e.g., one or more primers) of the disclosure for detecting an ABL1 fusion nucleic acid molecule of the disclosure in a sample, e.g., according to any detection method known in the art or described herein. In some embodiments of any of the kits or articles of manufacture provided herein, the kit or article of manufacture comprises a reagent (e.g., one or more oligonucleotides, primers, probes or baits of the present disclosure) for detecting a wild-type counterpart of an ABL1 fusion nucleic acid molecule of the disclosure (e.g., a wild type ABL1 gene, and/or a wild type fusion partner gene as described herein). In some embodiments, one or more oligonucleotides, primers, probes or baits are capable of hybridizing to an ABL1 fusion nucleic acid molecule of the disclosure, or to a wild-type counterpart of the ABL1 fusion nucleic acid molecule (e.g., a wild type ABL1 gene, and/or a wild type fusion partner gene as described herein). In some embodiments, the one or more oligonucleotides, primers, probes or baits of the present disclosure are capable of distinguishing an ABL1 fusion nucleic acid molecule of the disclosure, from a wild-type counterpart of the ABL1 fusion nucleic acid molecule (e.g., a wild type ABL1 gene, and/or a wild type fusion partner gene as described herein). In some embodiments, the kit is for use according to any method of detecting fusion nucleic acid molecules known in the art or described herein, such as sequencing, PCR, in situ hybridization methods, a nucleic acid hybridization assay, an amplification-based assay, a PCR-RFLP assay, real-time PCR, sequencing, next-generation sequencing, a screening analysis, FISH, spectral karyotyping, MFISH, comparative genomic hybridization, in situ hybridization, sequence-specific priming (SSP) PCR, HPLC, and mass-spectrometric genotyping. In some embodiments, a kit provided herein further comprises instructions for detecting an ABL1 fusion nucleic acid molecule of the disclosure, e.g., using one or more oligonucleotides, primers, probes or baits of the present disclosure.

In some embodiments, the kits or articles of manufacture comprise one or more antibodies or antibody fragments of the disclosure for detecting an ABL1 fusion polypeptide of the disclosure in a sample, e.g., according to any detection method known in the art or described herein. In some embodiments, the kit or article of manufacture comprises a reagent (e.g., one or more antibodies or antibody fragments of the present disclosure) for detecting the wild-type counterparts of an ABL1 fusion polypeptide provided herein (e.g., a wild type ABL1 polypeptide, and/or a wild type polypeptide encoded by a fusion partner gene as described herein). In some embodiments, the kits or articles of manufacture comprise one or more antibodies or antibody fragments of the present disclosure capable of binding to an ABL1 fusion polypeptide provided herein, or to wild-type counterparts of the ABL1 fusion polypeptide provided herein (e.g., a wild type ABL1 polypeptide, and/or a wild type polypeptide encoded by a fusion partner gene as described herein). In some embodiments, the kit is for use according to any protein or polypeptide detection assay known in the art or described herein, such as mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography). In some embodiments, the kit further comprises instructions for detecting an ABL1 fusion polypeptide of the disclosure, e.g., using one or more antibodies or antibody fragments of the present disclosure.

Further provided herein are kits or articles of manufacture comprising an anti-cancer therapy, such as an anti-cancer therapy described herein, e.g., an ABL1-targeted therapy, and a package insert comprising instructions for using the anti-cancer therapy in a method of treating or delaying progression of cancer, e.g., by administration to an individual from whom a sample comprising an ABL1 fusion nucleic acid molecule or polypeptide of the disclosure has been obtained. In some embodiments, the anti-cancer therapy is any of the anti-cancer therapies described herein for use in any of the methods for treating or delaying progression of cancer of the disclosure.

The kit or article of manufacture may include, for example, a container and a label or package insert on or associated with the container. Suitable containers include, for example, bottles, vials, syringes, and the like. The container may be formed from a variety of materials such as glass or plastic. The container holds or contains a composition comprising one or more reagents for detecting an ABL1 fusion nucleic acid molecule or polypeptide of the disclosure (e.g., one or more oligonucleotides, primers, probes, baits, antibodies or antibody fragments of the present disclosure) or one or more anti-cancer therapies of the disclosure. In some embodiments, the container holds or contains a composition comprising one or more anti-cancer therapies of the disclosure and may have a sterile access port (e.g., the container may be an intravenous solution bag or a vial having a stopper pierceable by a hypodermic injection needle).

The kit or article of manufacture may further include a second container comprising a diluent or buffer, e.g., a pharmaceutically-acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

The kit or article of manufacture of the present disclosure also includes information or instructions, for example in the form of a package insert, indicating that the one or more reagents and/or anti-cancer therapies are used for detecting an ABL1 fusion nucleic acid molecule or polypeptide of the disclosure, or for treating cancer, as described herein. The insert or label may take any form, such as paper or on electronic media such as a magnetically recorded medium (e.g., floppy disk), a CD-ROM, a Universal Serial Bus (USB) flash drive, and the like. The label or insert may also include other information concerning the pharmaceutical compositions and dosage forms in the kit or article of manufacture.

### V. Expression Vectors, Host Cells and Recombinant Cells

Provided herein are vectors comprising or encoding an ABL1 fusion nucleic acid molecule of the disclosure, or a bait, a probe, or an oligonucleotide described herein, or fragments thereof.

In some embodiments, a vector provided herein comprises or encodes an ABL1 fusion nucleic acid molecule of the disclosure, or a nucleic acid molecule encoding an ABL1 fusion polypeptide described herein.

In some embodiments, a vector provided herein is a nucleic acid molecule capable of transporting another nucleic acid to which it has been linked (e.g., ABL1 fusion nucleic acid molecules, baits, probes, or oligonucleotides described herein, or fragments thereof). In some embodiments, a vector is a plasmid, a cosmid or a viral vector. The vector may be capable of autonomous replication, or it can integrate into a host DNA. Viral vectors (e.g., comprising fusion nucleic acid molecules, baits, probes, or oligonucleotides described herein, or fragments thereof) are also contemplated herein, including, e.g., replication defective retroviruses, adenoviruses and adeno-associated viruses.

In some embodiments, a vector provided herein comprises an ABL1 fusion nucleic acid molecule, a bait, a probe, or an oligonucleotide of the disclosure in a form suitable for expression thereof in a host cell. In some embodiments, the vector includes one or more regulatory sequences operatively linked to the nucleotide sequence to be expressed. In some embodiments, the one or more regulatory sequences include promoters (e.g., promoters derived from polyoma, Adenovirus 2, cytomegalovirus and Simian Virus 40), enhancers, and other expression control elements (e.g., polyadenylation signals). In some embodiments, a regulatory sequence directs constitutive expression of a nucleotide sequence (e.g., ABL1 fusion nucleic acid molecules, baits, probes, or oligonucleotides described herein, or fragments thereof). In some embodiments, a regulatory sequence directs tissue-specific expression of a nucleotide sequence (e.g., ABL1 fusion nucleic acid molecules, baits, probes, or oligonucleotides described herein, or fragments thereof). In some embodiments, a regulatory sequence directs inducible expression of a nucleotide sequence (e.g., ABL1 fusion nucleic acid molecules, baits, probes, or oligonucleotides described herein, or fragments thereof). Examples of inducible regulatory sequences include, without limitation, promoters regulated by a steroid hormone, by a polypeptide hormone, or by a heterologous polypeptide, such as a tetracycline-inducible promoter. Examples of tissue- or cell-type-specific regulatory sequences include, without limitation, the albumin promoter, lymphoid-specific promoters, promoters of T cell receptors or immunoglobulins, neuron-specific promoters, pancreas-specific promoters, mammary gland-specific promoters, and developmentally-regulated promoters. In some embodiments, a vector provided herein comprises or encodes an ABL1 fusion nucleic acid molecule, a bait, a probe, or an oligonucleotide of the disclosure in the sense or the anti-sense orientation. In some embodiments, a vector (e.g., an expression vector) provided herein is introduced into host cells to thereby produce a polypeptide, e.g., an ABL1 fusion polypeptide described herein, or a fragment or mutant form thereof.

In some embodiments, the design of a vector provided herein depends on such factors as the choice of the host cell to be transformed, the level of expression desired, and the like. In some embodiments, expression vectors are designed for the expression of the ABL1 fusion nucleic acid molecules, baits, probes, or oligonucleotides described herein, or fragments thereof, in prokaryotic or eukaryotic cells, such as E. coli cells, insect cells (e.g., using baculovirus expression vectors), yeast cells, or mammalian cells. In some embodiments, a vector described herein is transcribed and translated in vitro, for example using T7 promoter regulatory sequences and T7 polymerase. In some embodiments, a vector (e.g., an expression vector) provided herein comprises or encodes an ABL1 fusion nucleic acid molecule described herein, wherein the nucleotide sequence of the fusion nucleic acid molecule described herein has been altered (e.g., codon optimized) so that the individual codons for each encoded amino acid are those preferentially utilized in the host cell.

Also provided herein are host cells, e.g., comprising ABL1 fusion nucleic acid molecules, ABL1 fusion polypeptides, baits, probes, vectors, or oligonucleotides of the disclosure. In some embodiments, a host cell (e.g., a recombinant host cell or recombinant cell) comprises a vector described herein (e.g., an expression vector described herein). In some embodiments, an ABL1 fusion nucleic acid molecule, bait, probe, vector, or oligonucleotide provided herein further includes sequences which allow it to integrate into the host cell's genome (e.g., through homologous recombination at a specific site). In some embodiments, a host cell provided herein is a prokaryotic or eukaryotic cell. Non-limiting examples of host cells include, without limitation, bacterial cells (e.g., E. coli), insect cells, yeast cells, or mammalian cells (e.g., human cells, rodent cells, mouse cells, rabbit cells, pig cells, Chinese hamster ovary cells (CHO), or COS cells, e.g., COS-7 cells, CV-1 origin SV40 cells). A host cell described herein includes the particular host cell, as well as the progeny of such a cell. Because certain modifications may occur in succeeding generations due to either mutation or environmental influences, such progeny may not, in fact, be identical to the parent host cell.

ABL1 fusion nucleic acid molecules, baits, probes, vectors, or oligonucleotides of the disclosure may be introduced into host cells using any suitable method known in the art, such as conventional transformation or transfection techniques (e.g., using calcium phosphate or calcium chloride co-precipitation, DEAE-dextran-mediated transfection, lipofection, or electroporation).

Also provided herein are methods of producing an ABL1 fusion polypeptide of the disclosure, e.g., by culturing a host cell described herein (e.g., into which a recombinant expression vector encoding a polypeptide has been introduced) in a suitable medium such that the fusion polypeptide is produced. In another embodiment, the method further includes isolating an ABL1 fusion polypeptide from the medium or the host cell.

### VI. Exemplary Embodiments

The following exemplary embodiments are representative of some aspects of the invention:

Exemplary Embodiment 1: A method for selecting a therapy for an individual having a cancer or for identifying an individual having a cancer who may benefit from a treatment comprising an Abelson tyrosine-protein kinase 1 (ABL1)-targeted therapy, the method comprising detecting in a sample from the individual an ABL1 fusion nucleic acid molecule, or an ABL1 fusion polypeptide encoded by the ABL1 fusion nucleic acid molecule, wherein the ABL1 fusion nucleic acid molecule comprises a fusion between an ABL1 gene, or a portion thereof, fused to:
(a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(b) a second gene, or a portion of a second gene, wherein the ABL1 fusion nucleic acid molecule comprises or results from an ABL1 breakpoint within any of exons 1-4 and 10, or introns 4-5, of ABL1;
(c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(d) a NUP214 gene, or a portion thereof, wherein the cancer is bone marrow leukemia lymphocytic chronic (CLL) or soft tissue liposarcoma;
(e) an AIF1L gene, or a portion thereof, wherein the cancer is soft tissue sarcoma, or ovary serous carcinoma;
(f) an RCSD1 gene, or a portion thereof, wherein the cancer is bone marrow leukemia lymphocytic chronic (CLL); or
(g) an EXOSC2 gene, or a portion thereof, wherein the cancer is peritoneum adenocarcinoma; wherein detection of the ABL1 fusion nucleic acid molecule, or the ABL1 fusion polypeptide encoded by the ABL1 fusion nucleic acid molecule, in the sample identifies the individual as one who may benefit from a treatment comprising an ABL1-targeted therapy.

Exemplary Embodiment 2: A method of identifying one or more treatment options for an individual having a cancer, the method comprising:
(1) detecting or acquiring knowledge of an ABL1 fusion nucleic acid molecule, or an ABL1 fusion polypeptide encoded by the ABL1 fusion nucleic acid molecule, in a sample from the individual, wherein the ABL1 fusion nucleic acid molecule comprises a fusion between an ABL1 gene, or a portion thereof, fused to:
(a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(b) a second gene, or a portion of a second gene, wherein the ABL1 fusion nucleic acid molecule comprises or results from an ABL1 breakpoint within any of exons 1-4 and 10, or introns 4-5, of ABL1;
(c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(d) a NUP214 gene, or a portion thereof, wherein the cancer is bone marrow leukemia lymphocytic chronic (CLL) or soft tissue liposarcoma;
(e) an AIF1L gene, or a portion thereof, wherein the cancer is soft tissue sarcoma, or ovary serous carcinoma;
(f) an RCSD1 gene, or a portion thereof, wherein the cancer is bone marrow leukemia lymphocytic chronic (CLL); or
(g) an EXOSC2 gene, or a portion thereof, wherein the cancer is peritoneum adenocarcinoma; and
(2) generating a report comprising one or more treatment options identified for the individual based, at least in part, on detection or on acquiring knowledge of the ABL1 fusion nucleic acid molecule, or the ABL1 fusion polypeptide encoded by the ABL1 fusion nucleic acid molecule, in the sample, wherein the one or more treatment options comprise an ABL1-targeted therapy.

Exemplary Embodiment 3: A method of selecting a treatment for an individual having a cancer, comprising acquiring knowledge of an ABL1 fusion nucleic acid molecule, or an ABL1 fusion polypeptide encoded by the ABL1 fusion nucleic acid molecule, in a sample from the individual, wherein the ABL1 fusion nucleic acid molecule comprises a fusion between an ABL1 gene, or a portion thereof, fused to:
(a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(b) a second gene, or a portion of a second gene, wherein the ABL1 fusion nucleic acid molecule comprises or results from an ABL1 breakpoint within any of exons 1-4 and 10, or introns 4-5, of ABL1;
(c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(d) a NUP214 gene, or a portion thereof, wherein the cancer is bone marrow leukemia lymphocytic chronic (CLL) or soft tissue liposarcoma;
(e) an AIF1L gene, or a portion thereof, wherein the cancer is soft tissue sarcoma, or ovary serous carcinoma;
(f) an RCSD1 gene, or a portion thereof, wherein the cancer is bone marrow leukemia lymphocytic chronic (CLL); or
(g) an EXOSC2 gene, or a portion thereof, wherein the cancer is peritoneum adenocarcinoma; wherein responsive to the acquisition of said knowledge: (i) the individual is classified as a candidate to receive a treatment comprising an ABL1-targeted therapy; and/or (ii) the individual is identified as likely to respond to a treatment that comprises an ABL1-targeted therapy.

Exemplary Embodiment 4: A method of predicting survival of an individual having a cancer, or an individual having a cancer treated with a treatment comprising an ABL1-targeted therapy, the method comprising acquiring knowledge of an ABL1 fusion nucleic acid molecule, or an ABL1 fusion polypeptide encoded by the ABL1 fusion nucleic acid molecule, in a sample from the individual, wherein the ABL1 fusion nucleic acid molecule comprises a fusion between an ABL1 gene, or a portion thereof, fused to:
(a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(b) a second gene, or a portion of a second gene, wherein the ABL1 fusion nucleic acid molecule comprises or results from an ABL1 breakpoint within any of exons 1-4 and 10, or introns 4-5, of ABL1;
(c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(d) a NUP214 gene, or a portion thereof, wherein the cancer is bone marrow leukemia lymphocytic chronic (CLL) or soft tissue liposarcoma;
(e) an AIF1L gene, or a portion thereof, wherein the cancer is soft tissue sarcoma, or ovary serous carcinoma;
(f) an RCSD1 gene, or a portion thereof, wherein the cancer is bone marrow leukemia lymphocytic chronic (CLL); or
(g) an EXOSC2 gene, or a portion thereof, wherein the cancer is peritoneum adenocarcinoma; wherein responsive to the acquisition of said knowledge, the individual is predicted to have longer survival when treated with a treatment comprising an ABL1-targeted therapy, as compared to survival of an individual whose cancer does not comprise an ABL1 fusion nucleic acid molecule or an ABL1 fusion polypeptide.

Exemplary Embodiment 5: A method of treating or delaying progression of a cancer, comprising:
(1) acquiring knowledge of an ABL1 fusion nucleic acid molecule, or an ABL1 fusion polypeptide encoded by the ABL1 fusion nucleic acid molecule, in a sample from an individual having a cancer, wherein the ABL1 fusion nucleic acid molecule comprises a fusion between an ABL1 gene, or a portion thereof, fused to:
(a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(b) a second gene, or a portion of a second gene, wherein the ABL1 fusion nucleic acid molecule comprises or results from an ABL1 breakpoint within any of exons 1-4 and 10, or introns 4-5, of ABL1;
(c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(d) a NUP214 gene, or a portion thereof, wherein the cancer is bone marrow leukemia lymphocytic chronic (CLL) or soft tissue liposarcoma;
(e) an AIF1L gene, or a portion thereof, wherein the cancer is soft tissue sarcoma, or ovary serous carcinoma;
(f) an RCSD1 gene, or a portion thereof, wherein the cancer is bone marrow leukemia lymphocytic chronic (CLL); or
(g) an EXOSC2 gene, or a portion thereof, wherein the cancer is peritoneum adenocarcinoma; and (2) responsive to said knowledge, administering to the individual an effective amount of a treatment that comprises an ABL1-targeted therapy.

Exemplary Embodiment 6: A method of treating or delaying progression of a cancer, comprising administering to an individual having a cancer an effective amount of a treatment that comprises an ABL1-targeted therapy, wherein the ABL1-targeted therapy is administered responsive to acquiring knowledge of an ABL1 fusion nucleic acid molecule, or an ABL1 fusion polypeptide encoded by the ABL1 fusion nucleic acid molecule, in a sample from the individual, wherein the ABL1 fusion nucleic acid molecule comprises a fusion between an ABL1 gene, or a portion thereof, fused to:
(a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(b) a second gene, or a portion of a second gene, wherein the ABL1 fusion nucleic acid molecule comprises or results from an ABL1 breakpoint within any of exons 1-4 and 10, or introns 4-5, of ABL1;
(c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(d) a NUP214 gene, or a portion thereof, wherein the cancer is bone marrow leukemia lymphocytic chronic (CLL) or soft tissue liposarcoma;
(e) an AIF1L gene, or a portion thereof, wherein the cancer is soft tissue sarcoma, or ovary serous carcinoma;
(f) an RCSD1 gene, or a portion thereof, wherein the cancer is bone marrow leukemia lymphocytic chronic (CLL); or
(g) an EXOSC2 gene, or a portion thereof, wherein the cancer is peritoneum adenocarcinoma.

Exemplary Embodiment 7: A method of monitoring, evaluating or screening an individual having a cancer, comprising acquiring knowledge of an ABL1 fusion nucleic acid molecule, or an ABL1 fusion polypeptide encoded by the ABL1 fusion nucleic acid molecule, in a sample from the individual, wherein the ABL1 fusion nucleic acid molecule comprises a fusion between an ABL1 gene, or a portion thereof, fused to:
(a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(b) a second gene, or a portion of a second gene, wherein the ABL1 fusion nucleic acid molecule comprises or results from an ABL1 breakpoint within any of exons 1-4 and 10, or introns 4-5, of ABL1;
(c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(d) a NUP214 gene, or a portion thereof, wherein the cancer is bone marrow leukemia lymphocytic chronic (CLL) or soft tissue liposarcoma;
(e) an AIF1L gene, or a portion thereof, wherein the cancer is soft tissue sarcoma, or ovary serous carcinoma;
(f) an RCSD1 gene, or a portion thereof, wherein the cancer is bone marrow leukemia lymphocytic chronic (CLL); or
(g) an EXOSC2 gene, or a portion thereof, wherein the cancer is peritoneum adenocarcinoma; wherein responsive to the acquisition of said knowledge, the individual is predicted to benefit from a treatment comprising an ABL1-targeted therapy and/or to have longer survival when treated with a treatment comprising an ABL1-targeted therapy, as compared to an individual whose cancer does not comprise an ABL1 fusion nucleic acid molecule or an ABL1 fusion polypeptide.

Exemplary Embodiment 8: A method of assessing an ABL1 fusion nucleic acid molecule, or an ABL1 fusion polypeptide, in a cancer in an individual, the method comprising:
(1) detecting an ABL1 fusion nucleic acid molecule, or an ABL1 fusion polypeptide encoded by the ABL1 fusion nucleic acid molecule, in a sample from the individual, wherein the ABL1 fusion nucleic acid molecule comprises a fusion between an ABL1 gene, or a portion thereof, fused to:
(a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(b) a second gene, or a portion of a second gene, wherein the ABL1 fusion nucleic acid molecule comprises or results from an ABL1 breakpoint within any of exons 1-4 and 10, or introns 4-5, of ABL1;
(c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(d) a NUP214 gene, or a portion thereof, wherein the cancer is bone marrow leukemia lymphocytic chronic (CLL) or soft tissue liposarcoma;
(e) an AIF1L gene, or a portion thereof, wherein the cancer is soft tissue sarcoma, or ovary serous carcinoma;
(f) an RCSD1 gene, or a portion thereof, wherein the cancer is bone marrow leukemia lymphocytic chronic (CLL); or
(g) an EXOSC2 gene, or a portion thereof, wherein the cancer is peritoneum adenocarcinoma; and
(2) providing an assessment of the ABL1 fusion nucleic acid molecule, or the ABL1 fusion polypeptide encoded by the ABL1 fusion nucleic acid molecule, in the sample.

Exemplary Embodiment 9: A method of detecting an ABL1 fusion nucleic acid molecule or an ABL1 fusion polypeptide in a cancer, the method comprising detecting an ABL1 fusion nucleic acid molecule, or an ABL1 fusion polypeptide encoded by the ABL1 fusion nucleic acid molecule, in a sample from an individual having a cancer, wherein the ABL1 fusion nucleic acid molecule comprises a fusion between an ABL1 gene, or a portion thereof, fused to:
(a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(b) a second gene, or a portion of a second gene, wherein the ABL1 fusion nucleic acid molecule comprises or results from an ABL1 breakpoint within any of exons 1-4 and 10, or introns 4-5, of ABL1;
(c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(d) a NUP214 gene, or a portion thereof, wherein the cancer is bone marrow leukemia lymphocytic chronic (CLL) or soft tissue liposarcoma;
(e) an AIF1L gene, or a portion thereof, wherein the cancer is soft tissue sarcoma, or ovary serous carcinoma;
(f) an RCSD1 gene, or a portion thereof, wherein the cancer is bone marrow leukemia lymphocytic chronic (CLL); or
(g) an EXOSC2 gene, or a portion thereof, wherein the cancer is peritoneum adenocarcinoma.

Exemplary Embodiment 10: A method of detecting the presence or absence of a cancer in an individual, the method comprising:
(1) detecting the presence or absence of a cancer in a sample from the individual; and
(2) detecting an ABL1 fusion nucleic acid molecule, or an ABL1 fusion polypeptide encoded by the ABL1 fusion nucleic acid molecule, in a sample from the individual, wherein the ABL1 fusion nucleic acid molecule comprises a fusion between an ABL1 gene, or a portion thereof, fused to:
(a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(b) a second gene, or a portion of a second gene, wherein the ABL1 fusion nucleic acid molecule comprises or results from an ABL1 breakpoint within any of exons 1-4 and 10, or introns 4-5, of ABL1;
(c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(d) a NUP214 gene, or a portion thereof, wherein the cancer is bone marrow leukemia lymphocytic chronic (CLL) or soft tissue liposarcoma;
(e) an AIF1L gene, or a portion thereof, wherein the cancer is soft tissue sarcoma, or ovary serous carcinoma;
(f) an RCSD1 gene, or a portion thereof, wherein the cancer is bone marrow leukemia lymphocytic chronic (CLL); or
(g) an EXOSC2 gene, or a portion thereof, wherein the cancer is peritoneum adenocarcinoma.

Exemplary Embodiment 11: The method of embodiment 10, comprising detecting the presence of the cancer in a sample from the individual; and/or detecting the presence of the ABL1 fusion nucleic acid molecule, or the ABL1 fusion polypeptide encoded by the ABL1 fusion nucleic acid molecule, in a sample from the individual.

Exemplary Embodiment 12: A method for monitoring progression or recurrence of a cancer in an individual, the method comprising:
(1) detecting, in a first sample obtained from the individual at a first time point, the presence or absence of an ABL1 fusion nucleic acid molecule, or an ABL1 fusion polypeptide encoded by the ABL1 fusion nucleic acid molecule;
(2) detecting, in a second sample obtained from the individual at a second time point after the first time point, the presence or absence of an ABL1 fusion nucleic acid molecule, or an ABL1 fusion polypeptide encoded by the ABL1 fusion nucleic acid molecule; and
(3) providing an assessment of cancer progression or cancer recurrence in the individual based, at least in part, on the presence or absence of the ABL1 fusion nucleic acid molecule, or the ABL1 fusion polypeptide encoded by the ABL1 fusion nucleic acid molecule, in the first sample and/or in the second sample;
wherein the ABL1 fusion nucleic acid molecule comprises a fusion between an ABL1 gene, or a portion thereof, fused to:
(a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(b) a second gene, or a portion of a second gene, wherein the ABL1 fusion nucleic acid molecule comprises or results from an ABL1 breakpoint within any of exons 1-4 and 10, or introns 4-5, of ABL1;
(c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(d) a NUP214 gene, or a portion thereof, wherein the cancer is bone marrow leukemia lymphocytic chronic (CLL) or soft tissue liposarcoma;
(e) an AIF1L gene, or a portion thereof, wherein the cancer is soft tissue sarcoma, or ovary serous carcinoma;
(f) an RCSD1 gene, or a portion thereof, wherein the cancer is bone marrow leukemia lymphocytic chronic (CLL); or
(g) an EXOSC2 gene, or a portion thereof, wherein the cancer is peritoneum adenocarcinoma.

Exemplary Embodiment 13: The method of embodiment 12, wherein the presence of the ABL1 fusion nucleic acid molecule, or the ABL1 fusion polypeptide encoded by the ABL1 fusion nucleic acid molecule, in the first sample and/or in the second sample identifies the individual as having decreased risk of cancer progression or cancer recurrence when treated with a treatment comprising an ABL1-targeted therapy.

Exemplary Embodiment 14: The method of embodiment 12 or embodiment 13, further comprising selecting a treatment, administering a treatment, adjusting a treatment, adjusting a dose of a treatment, or applying a treatment to the individual based, at least in part, on detecting the presence of the ABL1 fusion nucleic acid molecule, or the ABL1 fusion polypeptide encoded by the ABL1 fusion nucleic acid molecule, in the first sample and/or in the second sample, wherein the treatment comprises an ABL1-targeted therapy.

Exemplary Embodiment 15: A method of detecting an ABL1 fusion nucleic acid molecule, the method comprising:
(a) providing a plurality of nucleic acid molecules obtained from a sample from an individual having a cancer, wherein the plurality of nucleic acid molecules comprises nucleic acid molecules corresponding to an ABL1 fusion nucleic acid molecule, wherein the ABL1 fusion nucleic acid molecule comprises a fusion between an ABL1 gene, or a portion thereof, fused to:
(i) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(ii) a second gene, or a portion of a second gene, wherein the ABL1 fusion nucleic acid molecule comprises or results from an ABL1 breakpoint within any of exons 1-4 and 10, or introns 4-5, of ABL1;
(iii) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(iv) a NUP214 gene, or a portion thereof, wherein the cancer is bone marrow leukemia lymphocytic chronic (CLL) or soft tissue liposarcoma;
(v) an AIF1L gene, or a portion thereof, wherein the cancer is soft tissue sarcoma, or ovary serous carcinoma;
(vi) an RCSD1 gene, or a portion thereof, wherein the cancer is bone marrow leukemia lymphocytic chronic (CLL); or
(vii) an EXOSC2 gene, or a portion thereof, wherein the cancer is peritoneum adenocarcinoma;
(b) optionally, ligating one or more adapters onto one or more nucleic acid molecules from the plurality of nucleic acid molecules;
(c) optionally, amplifying the one or more ligated nucleic acid molecules from the plurality of nucleic acid molecules;
(d) optionally, capturing amplified nucleic acid molecules from the amplified nucleic acid molecules;
(e) sequencing, by a sequencer, the captured nucleic acid molecules to obtain a plurality of sequence reads that represent the captured nucleic acid molecules, wherein one or more of the plurality of sequence reads correspond to the ABL1 fusion nucleic acid molecule;
(f) analyzing the plurality of sequence reads for the presence or absence of the ABL1 fusion nucleic acid molecule; and
(g) based on the analyzing step, detecting the presence or absence of the ABL1 fusion nucleic acid molecule in the sample.

Exemplary Embodiment 16: The method of embodiment 15, wherein the sequencer comprises a next-generation sequencer.

Exemplary Embodiment 17: A method of detecting an ABL1 fusion nucleic acid molecule, the method comprising:
(a) providing a sample from an individual having a cancer, wherein the sample comprises a plurality of nucleic acid molecules;
(b) preparing a nucleic acid sequencing library from the plurality of nucleic acid molecules in the sample;
(c) amplifying said library;
(d) selectively enriching for one or more nucleic acid molecules comprising nucleotide sequences corresponding to an ABL1 fusion nucleic acid molecule in said library to produce an enriched sample, wherein the an ABL1 fusion nucleic acid molecule comprises a fusion between an ABL1 gene, or a portion thereof, fused to:
(i) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(ii) a second gene, or a portion of a second gene, wherein the ABL1 fusion nucleic acid molecule comprises or results from an ABL1 breakpoint within any of exons 1-4 and 10, or introns 4-5, of ABL1;
(iii) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(iv) a NUP214 gene, or a portion thereof, wherein the cancer is bone marrow leukemia lymphocytic chronic (CLL) or soft tissue liposarcoma;
(v) an AIF1L gene, or a portion thereof, wherein the cancer is soft tissue sarcoma (NOS), or ovary serous carcinoma;
(vi) an RCSD1 gene, or a portion thereof, wherein the cancer is bone marrow leukemia lymphocytic chronic (CLL); or
(vii) an EXOSC2 gene, or a portion thereof, wherein the cancer is peritoneum adenocarcinoma;
(e) sequencing the enriched sample, thereby producing a plurality of sequence reads;
(f) analyzing the plurality of sequence reads for the presence or absence of the ABL1 fusion nucleic acid molecule; and
(g) detecting, based on the analyzing step, the presence or absence of the ABL1 fusion nucleic acid molecule in the sample from the individual.

Exemplary Embodiment 18: The method of embodiment 15 or embodiment 16, wherein the one or more adapters comprise amplification primers, flow cell adapter sequences, substrate adapter sequences, sample index sequences, or unique molecular identifier (UMI) sequences.

Exemplary Embodiment 19: The method of embodiment 17, wherein the selectively enriching comprises: (a) combining one or more bait molecules with the library, thereby hybridizing the one or more bait molecules to one or more nucleic acid molecules comprising nucleotide sequences corresponding to the ABL1 fusion nucleic acid molecule, and producing nucleic acid hybrids; and (b) isolating the nucleic acid hybrids to produce the enriched sample.

Exemplary Embodiment 20: The method of embodiment 15 or embodiment 16, wherein the amplified nucleic acid molecules are captured by hybridization with one or more bait molecules.

Exemplary Embodiment 21: The method of any one of embodiments 15-20, wherein the amplifying comprises performing a polymerase chain reaction (PCR) amplification technique, a non-PCR amplification technique, or an isothermal amplification technique.

Exemplary Embodiment 22: The method of any one of embodiments 1-2 and 8-14, further comprising selectively enriching for one or more nucleic acid molecules in the sample comprising nucleotide sequences corresponding to the ABL1 fusion nucleic acid molecule; wherein the selectively enriching produces an enriched sample.

Exemplary Embodiment 23: The method of embodiment 22, wherein the selectively enriching comprises: (a) combining one or more bait molecules with the sample, thereby hybridizing the one or more bait molecules to one or more nucleic acid molecules in the sample comprising nucleotide sequences corresponding to the ABL1 fusion nucleic acid molecule and producing nucleic acid hybrids; and (b) isolating the nucleic acid hybrids to produce the enriched sample.

Exemplary Embodiment 24: The method of any one of embodiments 19-21 and 23, wherein the one or more bait molecules comprise a capture nucleic acid molecule configured to hybridize to a nucleotide sequence corresponding to the ABL1 fusion nucleic acid molecule.

Exemplary Embodiment 25: The method of embodiment 24, wherein the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides.

Exemplary Embodiment 26: The method of any one of embodiments 19-21 and 23-25, wherein the one or more bait molecules are conjugated to an affinity reagent or to a detection reagent.

Exemplary Embodiment 27: The method of embodiment 26, wherein the affinity reagent is an antibody, an antibody fragment, or biotin, or wherein the detection reagent is a fluorescent marker.

Exemplary Embodiment 28: The method of any one of embodiments 24-27, wherein the capture nucleic acid molecule comprises a DNA, RNA, or mixed DNA/RNA molecule.

Exemplary Embodiment 29: The method of any one of embodiments 17-18 and 22, wherein the selectively enriching comprises amplifying the one or more nucleic acid molecules comprising nucleotide sequences corresponding to the ABL1 fusion nucleic acid molecule using a polymerase chain reaction (PCR) to produce an enriched sample.

Exemplary Embodiment 30: The method of any one of embodiments 22-29, further comprising sequencing the enriched sample.

Exemplary Embodiment 31: The method of any one of embodiments 15-30, wherein the plurality of nucleic acid molecules comprises a mixture of cancer nucleic acid molecules and non-cancer nucleic acid molecules.

Exemplary Embodiment 32: The method of embodiment 31, wherein the cancer nucleic acid molecules are derived from a tumor portion of a heterogeneous tissue biopsy sample, and the non-cancer nucleic acid molecules are derived from a normal portion of the heterogeneous tissue biopsy sample.

Exemplary Embodiment 33: The method of embodiment 31, wherein the sample comprises a liquid biopsy sample, and wherein the cancer nucleic acid molecules are derived from a circulating tumor DNA (ctDNA) fraction of the liquid biopsy sample, and the non-cancer nucleic acid molecules are derived from a non-tumor fraction of the liquid biopsy sample.

Exemplary Embodiment 34: The method of any one of embodiments 15 and 17-33, wherein the sequencing comprises use of a massively parallel sequencing (MPS) technique, whole genome sequencing (WGS), whole exome sequencing, targeted sequencing, direct sequencing, or a Sanger sequencing technique; optionally wherein the sequencing comprises a massively parallel sequencing technique, and the massively parallel sequencing technique comprises next-generation sequencing (NGS).

Exemplary Embodiment 35: A method of identifying a candidate treatment for a cancer in an individual in need thereof, comprising: performing DNA sequencing on a sample obtained from the individual to determine a sequencing mutation profile, wherein the sequencing mutation profile identifies the presence or absence of an ABL1 fusion nucleic acid molecule in the sample, wherein the ABL1 fusion nucleic acid molecule comprises a fusion between an ABL1 gene, or a portion thereof, fused to:
(a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(b) a second gene, or a portion of a second gene, wherein the ABL1 fusion nucleic acid molecule comprises or results from an ABL1 breakpoint within any of exons 1-4 and 10, or introns 4-5, of ABL1;
(c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(d) a NUP214 gene, or a portion thereof, wherein the cancer is bone marrow leukemia lymphocytic chronic (CLL) or soft tissue liposarcoma;
(e) an AIF1L gene, or a portion thereof, wherein the cancer is soft tissue sarcoma, or ovary serous carcinoma;
(f) an RCSD1 gene, or a portion thereof, wherein the cancer is bone marrow leukemia lymphocytic chronic (CLL); or
(g) an EXOSC2 gene, or a portion thereof, wherein the cancer is peritoneum adenocarcinoma; and selecting a treatment for the individual based, at least in part, on the sequencing mutation profile, wherein the treatment comprises an ABL1-targeted therapy.

Exemplary Embodiment 36: The method of embodiment 35, wherein the presence of the ABL1 fusion nucleic acid molecule in the sample identifies the individual as one who may benefit from a treatment comprising an ABL1-targeted therapy; and/or predicts the individual to have longer survival when treated with a treatment comprising an ABL1-targeted therapy, as compared to survival of an individual whose cancer does not comprise an ABL1 fusion nucleic acid molecule.

Exemplary Embodiment 37: The method of embodiment 35 or embodiment 36, wherein the sequencing comprises use of a massively parallel sequencing (MPS) technique, whole genome sequencing (WGS), whole exome sequencing, targeted sequencing, direct sequencing, or a Sanger sequencing technique; optionally wherein the sequencing comprises a massively parallel sequencing technique, and the massively parallel sequencing technique comprises next generation sequencing (NGS).

Exemplary Embodiment 38: The method of any one of embodiments 35-37, wherein the sequencing mutation profile identifies the presence or absence of a fragment of the ABL1 fusion nucleic acid molecule comprising a breakpoint or fusion junction.

Exemplary Embodiment 39: A method of treating or delaying progression of cancer, comprising: detecting in a sample from an individual having a cancer an ABL1 fusion nucleic acid molecule, or an ABL1 fusion polypeptide encoded by the ABL1 fusion nucleic acid molecule, wherein the ABL1 fusion nucleic acid molecule comprises a fusion between an ABL1 gene, or a portion thereof, fused to:
(a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(b) a second gene, or a portion of a second gene, wherein the ABL1 fusion nucleic acid molecule comprises or results from an ABL1 breakpoint within any of exons 1-4 and 10, or introns 4-5, of ABL1;
(c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(d) a NUP214 gene, or a portion thereof, wherein the cancer is bone marrow leukemia lymphocytic chronic (CLL) or soft tissue liposarcoma;
(e) an AIF1L gene, or a portion thereof, wherein the cancer is soft tissue sarcoma, or ovary serous carcinoma;
(f) an RCSD1 gene, or a portion thereof, wherein the cancer is bone marrow leukemia lymphocytic chronic (CLL); or
(g) an EXOSC2 gene, or a portion thereof, wherein the cancer is peritoneum adenocarcinoma; and administering to the individual an effective amount of a treatment that comprises an ABL1-targeted therapy.

Exemplary Embodiment 40: The method of any one of embodiments 1-39, further comprising generating a report, wherein the report: (a) indicates the presence of the ABL1 fusion nucleic acid molecule, or the ABL1 fusion polypeptide encoded by the ABL1 fusion nucleic acid molecule, in the sample from the individual; and/or (b) indicates a treatment or one or more treatment options identified or selected for the individual based, at least in part, on the presence of the ABL1 fusion nucleic acid molecule, or the ABL1 fusion polypeptide encoded by the ABL1 fusion nucleic acid molecule, in the sample from the individual, wherein the treatment or the one or more treatment options comprise an ABL1-targeted therapy.

Exemplary Embodiment 41: The method of any one of embodiments 1-40, further comprising generating a molecular profile for the individual, based, at least in part, on detecting or acquiring knowledge of the ABL1 fusion nucleic acid molecule, or the ABL1 fusion polypeptide encoded by the ABL1 fusion nucleic acid molecule, in the sample from the individual.

Exemplary Embodiment 42: The method of embodiment 41, wherein the molecular profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof.

Exemplary Embodiment 43: The method of embodiment 41 or embodiment 42, wherein the molecular profile for the individual further comprises results from a nucleic acid sequencing-based test.

Exemplary Embodiment 44: The method of any one of embodiments 41-43, further comprising selecting a treatment, administering a treatment, or applying a treatment to the individual based on the generated molecular profile, wherein the treatment comprises an ABL1-targeted therapy.

Exemplary Embodiment 45: The method of any one of embodiments 41-44, further comprising generating a report, wherein the report comprises the molecular profile for the individual.

Exemplary Embodiment 46: The method of embodiment 45, wherein the report further comprises information on a treatment or one or more treatment options identified or selected for the individual based, at least in part, on the molecular profile for the individual, wherein the treatment or one or more treatment options comprise an ABL1-targeted therapy.

Exemplary Embodiment 47: The method of any one of embodiments 2, 22-34, and 40-46, further comprising providing the report to the individual, a caregiver, a healthcare provider, a physician, an oncologist, an electronic medical record system, a hospital, a clinic, a third-party payer, an insurance company, or a government office.

Exemplary Embodiment 48: The method of any one of embodiments 1-47, wherein the individual is a human.

Exemplary Embodiment 49: The method of any one of embodiments 1-48, further comprising obtaining the sample from the individual.

Exemplary Embodiment 50: The method of any one of embodiments 1-49, wherein the sample is obtained or derived from the cancer.

Exemplary Embodiment 51: The method of any one of embodiments 1-50, wherein the sample comprises a tissue biopsy sample, a liquid biopsy sample, or a normal control.

Exemplary Embodiment 52: The method of any one of embodiments 1-50, wherein the sample is from a tumor biopsy, tumor specimen, or circulating tumor cell.

Exemplary Embodiment 53: The method of any one of embodiments 1-50, wherein the sample is a liquid biopsy sample comprising blood, plasma, cerebrospinal fluid, sputum, stool, urine, or saliva.

Exemplary Embodiment 54: The method of any one of embodiments 1-53, wherein the sample comprises cells and/or nucleic acids from the cancer.

Exemplary Embodiment 55: The method of embodiment 54, wherein the sample comprises mRNA, DNA, circulating tumor DNA (ctDNA), cell-free DNA, or cell-free RNA from the cancer.

Exemplary Embodiment 56: The method of any one of embodiments 1-50, wherein the sample is a liquid biopsy sample comprising circulating tumor cells (CTCs).

Exemplary Embodiment 57: The method of any one of embodiments 1-50, wherein the sample is a liquid biopsy sample comprising cell-free DNA (cfDNA), circulating tumor DNA (ctDNA), or any combination thereof.

Exemplary Embodiment 58: The method of any one of embodiments 2-7 and 40-57, wherein the acquiring knowledge of the ABL1 fusion nucleic acid molecule, or the ABL1 fusion polypeptide encoded by the ABL1 fusion nucleic acid molecule, comprises detecting the ABL1 fusion nucleic acid molecule, or the ABL1 fusion polypeptide encoded by the ABL1 fusion nucleic acid molecule, in the sample.

Exemplary Embodiment 59: The method of any one of embodiments 1-2, 8-34, and 39-58, wherein detecting the ABL1 fusion nucleic acid molecule, or the ABL1 fusion polypeptide encoded by the ABL1 fusion nucleic acid molecule, in the sample comprises detecting a fragment of the ABL1 fusion nucleic acid molecule, or of the ABL1 fusion polypeptide encoded by the ABL1 fusion nucleic acid molecule, comprising a breakpoint or fusion junction.

Exemplary Embodiment 60: The method of any one of embodiments 1-2, 8-14, and 39-59, wherein the ABL1 fusion nucleic acid molecule is detected in the sample by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing.

Exemplary Embodiment 61: The method of embodiment 60, wherein the sequencing comprises a massively parallel sequencing (MPS) technique, whole genome sequencing (WGS), whole exome sequencing, targeted sequencing, direct sequencing, or a Sanger sequencing technique; optionally wherein the massively parallel sequencing (MPS) technique comprises next-generation sequencing (NGS).

Exemplary Embodiment 62: The method of any one of embodiments 1-2, 8-14, and 39-59, wherein the ABL1 fusion polypeptide encoded by the ABL1 fusion nucleic acid molecule is detected in the sample by one or more of: immunoblotting, enzyme linked immunosorbent assay (ELISA), immunohistochemistry, or mass spectrometry.

Exemplary Embodiment 63: The method of any one of embodiments 1-14, 39-59, and 62, wherein the ABL1 fusion polypeptide encoded by the ABL1 fusion nucleic acid molecule comprises an ABL1 kinase domain, or a fragment of an ABL1 kinase domain, having a kinase activity; optionally wherein the kinase activity is constitutive.

Exemplary Embodiment 64: The method of any one of embodiments 1-14, 39-59, and 62-63, wherein the ABL1 fusion polypeptide encoded by the ABL1 fusion nucleic acid molecule is oncogenic; optionally wherein the ABL1 fusion polypeptide encoded by the ABL1 fusion nucleic acid molecule promotes cancer cell survival, angiogenesis, cancer cell proliferation, and any combination thereof.

Exemplary Embodiment 65: The method of any one of embodiments 1-7, 13-14, and 35-64, wherein the ABL1-targeted therapy comprises one or more of a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer comprising an ABL1 gene fusion or rearrangement, an ABL1-targeted therapy being tested in a clinical trial, a treatment for cancer comprising an ABL1 gene fusion or rearrangement being tested in a clinical trial, or any combination thereof.

Exemplary Embodiment 66: The method of any one of embodiments 1-7, 13-14, and 35-65, wherein the ABL1-targeted therapy is a kinase inhibitor.

Exemplary Embodiment 67: The method of embodiment 66, wherein the ABL1-targeted therapy is a tyrosine kinase inhibitor.

Exemplary Embodiment 68: The method of embodiment 66 or embodiment 67, wherein the ABL1-targeted therapy is kinase inhibitor that inhibits the kinase activity of an ABL1 polypeptide.

Exemplary Embodiment 69: The method of any one of embodiments 66-68, wherein the ABL1-targeted therapy is a multi-kinase inhibitor or an ABL1-specific inhibitor.

Exemplary Embodiment 70: The method of any one of embodiments 66-69, wherein the ABL1-targeted therapy comprises one or more of imatinib, nilotinib, dasatinib, ponatinib, bosutinib, asciminib, olverembatinib, radotinib or vodobatinib.

Exemplary Embodiment 71: The method of embodiment 65, wherein the nucleic acid inhibits the expression of the ABL1 fusion nucleic acid molecule, or the ABL1 fusion polypeptide encoded by the ABL1 fusion nucleic acid molecule.

Exemplary Embodiment 72: The method of embodiment 71, wherein the nucleic acid is a double-stranded RNA (dsRNA), a small interfering RNA (siRNA), or a small hairpin RNA (shRNA).

Exemplary Embodiment 73: The method of embodiment 65, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Embodiment 74: The method of any one of embodiments 1-73, wherein the individual has received a prior anti-cancer treatment, or is being treated with an anti-cancer treatment; optionally wherein the cancer is resistant or refractory to the anti-cancer treatment.

Exemplary Embodiment 75: The method of any one of embodiments 1-7, 13-14, and 35-74, wherein the treatment or the one or more treatment options further comprise an additional anti-cancer therapy.

Exemplary Embodiment 76: The method of embodiment 75, wherein the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

Exemplary Embodiment 77: The method of embodiment 76, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Embodiment 78: The method of embodiment 76, wherein the nucleic acid comprises a double-stranded RNA (dsRNA), a small interfering RNA (siRNA), or a small hairpin RNA (shRNA).

Exemplary Embodiment 79: The method of any one of embodiments 1-78, wherein the cancer is metastatic.

Exemplary Embodiment 80: The method of any one of embodiments 1-79, wherein the ABL1 fusion nucleic acid molecule comprises a fusion between an ABL1 gene, or a portion thereof, fused to any one of genes APOOL, BMP2K, DNAH2, RHEX/C1orf186, EHMT1, MED27, RALGPS1, RFFL, SLC27A4, TSC1, TSPAN18, or ZNF804B, or a portion thereof, wherein the cancer is a carcinoma, a sarcoma, a lymphoma, a leukemia, a myeloma, a germ cell cancer, or a blastoma.

Exemplary Embodiment 81: The method of any one of embodiments 1-79, wherein the ABL1 fusion nucleic acid molecule comprises a fusion between an ABL1 gene, or a portion thereof, fused to a second gene, or a portion of a second gene, wherein the ABL1 fusion nucleic acid molecule comprises or results from an ABL1 breakpoint within any of exons 1-4 and 10, or introns 4-5, of ABL1, wherein the cancer is a carcinoma, a sarcoma, a lymphoma, a leukemia, a myeloma, a germ cell cancer, or a blastoma.

Exemplary Embodiment 82: The method of embodiment 80 or embodiment 81, wherein the cancer is a solid tumor or a hematologic malignancy.

Exemplary Embodiment 83: The method of any one of embodiments 80-82, wherein the cancer is a B cell cancer, multiple myeloma, bone marrow multiple myeloma, melanoma, breast cancer, lung cancer, bronchus cancer, colorectal cancer, prostate cancer, pancreatic cancer, stomach cancer, ovarian cancer, ovary epithelial carcinoma, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, testis germ cell tumor, biliary tract cancer, small bowel cancer, appendix cancer, salivary gland cancer, thyroid gland cancer, adrenal gland cancer, osteosarcoma, soft tissue osteosarcoma, chondrosarcoma, a cancer of hematological tissue, an adenocarcinoma, an inflammatory myofibroblastic tumor, a gastrointestinal stromal tumor (GIST), colon cancer, myelodysplastic syndrome (MDS), myeloproliferative disorder (MPD), acute lymphocytic leukemia (ALL) or bone marrow leukemia lymphocytic acute (ALL), bone marrow leukemia T cell acute (T-ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinoma, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, squamous cell cancer, non-squamous cell cancer, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, lung adenocarcinoma, non-small cell lung cancer, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, peritoneum adenocarcinoma, soft tissue malignant peripheral nerve sheath tumor (MPNST) or a carcinoid tumor.

Exemplary Embodiment 84: The method of any one of embodiments 1-80 and 82-83, wherein the ABL1 fusion nucleic acid molecule comprises a fusion between an ABL1 gene, or a portion thereof, fused to an APOOL gene, or a portion thereof, and wherein the ABL1 fusion nucleic acid molecule:
(a) comprises, in 5′ to 3′ direction, an APOOL gene, or a portion thereof, fused to an ABL1 gene, or a portion thereof;
(b) comprises or results from a breakpoint within intron 2 of an APOOL gene, and/or exon 2 of an ABL1 gene;
(c) comprises a fusion between exon 2, or a portion thereof, of an APOOL gene fused to exon 2, or a portion thereof, of an ABL1 gene;
(d) comprises or results from a 5′ breakpoint within chromosomal coordinates chrX: 84302705-84302904 and/or a 3′ breakpoint within chromosomal coordinates chr9: 133729584-133729766; and/or
(e) comprises, in 5′ to 3′ direction, exon 1 and exon 2, or a portion thereof, of an APOOL gene, fused to exon 2, or a portion thereof, and exons 3-11 of an ABL1 gene;
optionally, wherein the cancer is a lung adenocarcinoma.

Exemplary Embodiment 85: The method of any one of embodiments 1-14, 39-59, 62-80, and 82-84, wherein the ABL1 fusion polypeptide encoded by the ABL1 fusion nucleic acid molecule comprises a fusion between an ABL1 polypeptide, or a portion thereof, fused to an APOOL polypeptide, or a portion thereof, and wherein the ABL1 fusion polypeptide:
(a) comprises, in the N- to C-terminus direction, an APOOL polypeptide, or a portion thereof, fused to an ABL1 polypeptide, or a portion thereof;
(b) results from a breakpoint within intron 2 of an APOOL gene, and/or exon 2 of an ABL1 gene;
(c) comprises a fusion between an amino acid sequence encoded by exon 2, or a portion thereof, of an APOOL gene fused to an amino acid sequence encoded by exon 2, or a portion thereof, of an ABL1 gene;
(d) results from a 5′ breakpoint within chromosomal coordinates chrX: 84302705-84302904 and/or a 3′ breakpoint within chromosomal coordinates chr9: 133729584-133729766; and/or
(e) comprises, in the N- to C-terminus direction, an amino acid sequence encoded by exon 1 and exon 2, or a portion thereof, of APOOL, fused to an amino acid sequence encoded by exon 2, or a portion thereof, and exons 3-11 of ABL1;
optionally, wherein the cancer is a lung adenocarcinoma.

Exemplary Embodiment 86: The method of any one of embodiments 1-80 and 82-83, wherein the ABL1 fusion nucleic acid molecule comprises a fusion between an ABL1 gene, or a portion thereof, fused to an BMP2K gene, or a portion thereof, and wherein the ABL1 fusion nucleic acid molecule:
(a) comprises, in 5′ to 3′ direction, a BMP2K gene, or a portion thereof, fused to an ABL1 gene, or a portion thereof;
(b) comprises a fusion between exon 15, or a portion thereof, of a BMP2K gene fused to exon 3, or a portion thereof, of an ABL1 gene;
(c) comprises or results from a 5′ breakpoint within chromosomal coordinates chr4: 79800022-79800062 and/or a 3′ breakpoint within chromosomal coordinates chr9: 133730205-133730245; and/or
(d) comprises, in 5′ to 3′ direction, exons 1-14 and exon 15, or a portion thereof, of a BMP2K gene, fused to exon 3, or a portion thereof, and exons 4-11 of an ABL1 gene;
optionally, wherein the cancer is a bone marrow leukemia T cell acute (T-ALL).

Exemplary Embodiment 87: The method of any one of embodiments 1-14, 39-59, 62-80, and 82-83 and 86, wherein the ABL1 fusion polypeptide encoded by the ABL1 fusion nucleic acid molecule comprises a fusion between an ABL1 polypeptide, or a portion thereof, fused to a BMP2K polypeptide, or a portion thereof, and wherein the ABL1 fusion polypeptide:
(a) comprises, in the N- to C-terminus direction, a BMP2K polypeptide, or a portion thereof, fused to an ABL1 polypeptide, or a portion thereof;
(b) comprises a fusion between an amino acid sequence encoded by exon 15, or a portion thereof, of a BMP2K gene fused to an amino acid sequence encoded by exon 3, or a portion thereof, of an ABL1 gene;
(c) results from a 5′ breakpoint within chromosomal coordinates chr4: 79800022-79800062 and/or a 3′ breakpoint within chromosomal coordinates chr9: 133730205-133730245; and/or
(d) comprises, in the N- to C-terminus direction, an amino acid sequence encoded by exons 1-14 and exon 15, or a portion thereof, of a BMP2K gene, fused to an amino acid sequence encoded by exon 3, or a portion thereof, and exons 4-11 of an ABL1 gene;
optionally, wherein the cancer is a bone marrow leukemia T cell acute (T-ALL).

Exemplary Embodiment 88: The method of any one of embodiments 1-80 and 82-83, wherein the ABL1 fusion nucleic acid molecule comprises a fusion between an ABL1 gene, or a portion thereof, fused to a DNAH2 gene, or a portion thereof, and wherein the ABL1 fusion nucleic acid molecule:
(a) comprises, in 5′ to 3′ direction, an ABL1 gene, or a portion thereof, fused to a DNAH2 gene, or a portion thereof;
(b) comprises a fusion between exon 10, or a portion thereof, of an ABL1 gene fused to exon 4, or a portion thereof, of a DNAH2 gene;
(c) comprises or results from a 5′ breakpoint within chromosomal coordinates chr9: 133755953-133755993 and/or a 3′ breakpoint within chromosomal coordinates chr17: 7636399-7636439; and/or
(d) comprises, in 5′ to 3′ direction, exons 1-9 and exon 10, or a portion thereof, of an ABL1 gene, fused to exon 4, or a portion thereof, and exons 5-85 of a DNAH2 gene;
optionally, wherein the cancer is a soft tissue sarcoma, and optionally wherein the soft tissue sarcoma is not otherwise specified (NOS).

Exemplary Embodiment 89: The method of any one of embodiments 1-14, 39-59, 62-80, 82-83, and 88, wherein the ABL1 fusion polypeptide encoded by the ABL1 fusion nucleic acid molecule comprises a fusion between an ABL1 polypeptide, or a portion thereof, fused to a DNAH2 polypeptide, or a portion thereof, and wherein the ABL1 fusion polypeptide:
(a) comprises, in the N- to C-terminus direction, an ABL1 polypeptide, or a portion thereof, fused to a DNAH2 polypeptide, or a portion thereof;
(b) comprises a fusion between an amino acid sequence encoded by exon 10, or a portion thereof, of an ABL1 gene fused to an amino acid sequence encoded by exon 4, or a portion thereof, of a DNAH2 gene;
(c) results from a 5′ breakpoint within chromosomal coordinates chr9: 133755953-133755993 and/or a 3′ breakpoint within chromosomal coordinates chr17: 7636399-7636439; and/or
(d) comprises, in the N- to C-terminus direction, an amino acid sequence encoded by exons 1-9 and exon 10, or a portion thereof, of an ABL1 gene, fused to an amino acid sequence encoded by exon 4, or a portion thereof, and exons 5-85 of a DNAH2 gene;
optionally, wherein the cancer is a soft tissue sarcoma, and optionally wherein the soft tissue sarcoma is not otherwise specified (NOS).

Exemplary Embodiment 90: The method of any one of embodiments 1-80 and 82-83, wherein the ABL1 fusion nucleic acid molecule comprises a fusion between an ABL1 gene, or a portion thereof, fused to a RHEX/Clorf186 gene, or a portion thereof, and wherein the ABL1 fusion nucleic acid molecule:
(a) comprises, in 5′ to 3′ direction a RHEX/Clorf186 gene, or a portion thereof, fused to an ABL1 gene, or a portion thereof;
(b) comprises a fusion between exon 1, or a portion thereof, of a RHEX/Clorf186 gene fused to exon 2, or a portion thereof, of an ABL1 gene;
(c) comprises or results from a 5′ breakpoint within chromosomal coordinates chr1: 206288099-206288139 and/or a 3′ breakpoint within chromosomal coordinates chr9: 133729488-133729528; and/or
(d) comprises, in 5′ to 3′ direction, exon 1, or a portion thereof, of an RHEX/C1orf186 gene, fused to exon 2, or a portion thereof, and exons 3-11 of ABL1;
optionally, wherein the cancer is a bone marrow multiple myeloma.

Exemplary Embodiment 91: The method of any one of embodiments 1-14, 39-59, 62-80, 82-83, and 90, wherein the ABL1 fusion polypeptide encoded by the ABL1 fusion nucleic acid molecule comprises a fusion between an ABL1 polypeptide, or a portion thereof, fused to a RHEX/C1orf186 polypeptide, or a portion thereof, and wherein the ABL1 fusion polypeptide:
(a) comprises, in the N- to C-terminus direction, a RHEX/C1orf186 polypeptide, or a portion thereof, fused to an ABL1 polypeptide, or a portion thereof;
(b) comprises a fusion between an amino acid sequence encoded by exon 1, or a portion thereof, of a RHEX/C1orf186 gene fused to an amino acid sequence encoded by exon 2, or a portion thereof, of an ABL1 gene;
(c) results from a 5′ breakpoint within chromosomal coordinates chr1: 206288099-206288139 and/or a 3′ breakpoint within chromosomal coordinates chr9: 133729488-133729528; and/or
(d) comprises, in the N- to C-terminus direction, an amino acid sequence encoded by exon 1, or a portion thereof, of an RHEX/Clorf186 gene, fused to an amino acid sequence encoded by exon 2, or a portion thereof, and exons 3-11 of an ABL1 gene;
optionally, wherein the cancer is a bone marrow multiple myeloma.

Exemplary Embodiment 92: The method of any one of embodiments 1-80 and 82-83, wherein the ABL1 fusion nucleic acid molecule comprises a fusion between an ABL1 gene, or a portion thereof, fused to an EHMT1 gene, or a portion thereof, and wherein the ABL1 fusion nucleic acid molecule:
(a) comprises, in 5′ to 3′ direction an EHMT1 gene, or a portion thereof, fused to an ABL1 gene, or a portion thereof;
(b) comprises a fusion between exon 4, or a portion thereof, of an EHMT1 gene fused to exon 2, or a portion thereof, of an ABL1 gene;
(c) comprises or results from a breakpoint within intron 4 of an EHMT1 gene and/or a breakpoint within intron 1 of an ABL1 gene;
(d) comprises or results from a 5′ breakpoint within chromosomal coordinates chr9: 140630475-140630606 and/or a 3′ breakpoint within chromosomal coordinates chr9: 133729408-133729648;
(e) comprises, in 5′ to 3′ direction, exons 1-3 and exon 4, or a portion thereof, of an EHMT1 gene, fused to exon 2, or a portion thereof, and exons 3-11 of an ABL1 gene;
(f) comprises the nucleotide sequence of SEQ ID NO: 2, or a nucleotide sequence having at least about 70% homology thereto; and/or
(g) encodes an ABL1 fusion polypeptide comprising the amino acid sequence of SEQ ID NO: 23, or an amino acid sequence having at least about 70% homology thereto;
optionally, wherein the cancer is a lung adenocarcinoma.

Exemplary Embodiment 93: The method of any one of embodiments 1-14, 39-59, 62-80, 82-83, and 92, wherein the ABL1 fusion polypeptide encoded by the ABL1 fusion nucleic acid molecule comprises a fusion between an ABL1 polypeptide, or a portion thereof, fused to an EHMT1 polypeptide, or a portion thereof, and wherein the ABL1 fusion polypeptide:
(a) comprises, in the N- to C-terminus direction, an EHMT1 polypeptide, or a portion thereof, fused to an ABL1 polypeptide, or a portion thereof;
(b) comprises a fusion between an amino acid sequence encoded by exon 4, or a portion thereof, of an EHMT1 gene fused to an amino acid sequence encoded by exon 2, or a portion thereof, of an ABL1 gene;
(c) results from a breakpoint within intron 4 of an EHMT1 gene and/or a breakpoint within intron 1 of an ABL1 gene;
(d) results from a 5′ breakpoint within chromosomal coordinates chr9: 140630475-140630606 and/or a 3′ breakpoint within chromosomal coordinates chr9: 133729408-133729648;
(e) comprises, in the N- to C-terminus direction, an amino acid sequence encoded by exons 1-3 and exon 4, or a portion thereof, of an EHMT1 gene, fused to an amino acid sequence encoded by exon 2, or a portion thereof, and exons 3-11 of an ABL1 gene;
(f) is encoded by the nucleotide sequence of SEQ ID NO: 2, or a nucleotide sequence having at least about 70% homology thereto; and/or
(g) comprises the amino acid sequence of SEQ ID NO: 23, or an amino acid sequence having at least about 70% homology thereto;
optionally, wherein the cancer is a lung adenocarcinoma.

Exemplary Embodiment 94: The method of any one of embodiments 1-80 and 82-83, wherein the ABL1 fusion nucleic acid molecule comprises a fusion between an ABL1 gene, or a portion thereof, fused to a MED27 gene, or a portion thereof, and wherein the ABL1 fusion nucleic acid molecule:
(a) comprises, in 5′ to 3′ direction a MED27 gene, or a portion thereof, fused to an ABL1 gene, or a portion thereof;
(b) comprises a fusion between exon 4, or a portion thereof, of a MED27 gene fused to exon 5, or a portion thereof, of an ABL1 gene;
(c) comprises or results from a breakpoint within intron 4 of a MED27 gene and/or a breakpoint within intron 4 of an ABL1 gene;
(d) comprises or results from a 5′ breakpoint within chromosomal coordinates chr9: 134774423-134776006 and/or a 3′ breakpoint within chromosomal coordinates chr9: 133747364-133747762;
(e) comprises, in 5′ to 3′ direction, exons 1-3 and exon 4, or a portion thereof, of a MED27 gene fused to exon 5, or a portion thereof, and exons 6-11 of an ABL1 gene;
(f) comprises the nucleotide sequence of SEQ ID NO: 3, or a nucleotide sequence having at least about 70% homology thereto; and/or
(g) encodes an ABL1 fusion polypeptide comprising the amino acid sequence of SEQ ID NO: 24, or an amino acid sequence having at least about 70% homology thereto;
optionally, wherein the cancer is an ovary epithelial carcinoma, and optionally wherein the ovary epithelial carcinoma is not otherwise specified (NOS).

Exemplary Embodiment 95: The method of any one of embodiments 1-14, 39-59, 62-80, 82-83, and 94, wherein the ABL1 fusion polypeptide encoded by the ABL1 fusion nucleic acid molecule comprises a fusion between an ABL1 polypeptide, or a portion thereof, fused to a MED27 polypeptide, or a portion thereof, and wherein the ABL1 fusion polypeptide:
(a) comprises, in the N- to C-terminus direction, a MED27 polypeptide, or a portion thereof, fused to an ABL1 polypeptide, or a portion thereof;
(b) comprises a fusion between an amino acid sequence encoded by exon 4, or a portion thereof, of a MED27 gene fused to an amino acid sequence encoded by exon 5, or a portion thereof, of an ABL1 gene;
(c) results from a breakpoint within intron 4 of a MED27 gene and/or a breakpoint within intron 4 of an ABL1 gene;
(d) results from a 5′ breakpoint within chromosomal coordinates chr9: 134774423-134776006 and/or a 3′ breakpoint within chromosomal coordinates chr9: 133747364-133747762;
(e) comprises, in the N- to C-terminus direction, an amino acid sequence encoded by exons 1-3 and exon 4, or a portion thereof, of a MED27 gene, fused to an amino acid sequence encoded by exon 5, or a portion thereof, and exons 6-11 of an ABL1 gene;
(f) is encoded by the nucleotide sequence of SEQ ID NO: 3, or a nucleotide sequence having at least about 70% homology thereto; and/or
(g) comprises the amino acid sequence of SEQ ID NO: 24, or an amino acid sequence having at least about 70% homology thereto;
optionally, wherein the cancer is an ovary epithelial carcinoma, and optionally wherein the ovary epithelial carcinoma is not otherwise specified (NOS).

Exemplary Embodiment 96: The method of any one of embodiments 1-80 and 82-83, wherein the ABL1 fusion nucleic acid molecule comprises a fusion between an ABL1 gene, or a portion thereof, fused to a RALGPS1 gene, or a portion thereof, and wherein the ABL1 fusion nucleic acid molecule:
(a) comprises, in 5′ to 3′ direction, a RALGPS1 gene, or a portion thereof, fused to an ABL1 gene, or a portion thereof;
(b) comprises a fusion between exon 1, or a portion thereof, of a RALGPS1 gene fused to exon 3, or a portion thereof, of an ABL1 gene;
(c) comprises or results from a breakpoint within intron 1 of a RALGPS1 gene and/or a breakpoint within intron 2 of an ABL1 gene;
(d) comprises or results from a 5′ breakpoint within chromosomal coordinates chr9: 129694023-129694282 and/or a 3′ breakpoint within chromosomal coordinates chr9: 133730118-133730529; and/or
(e) comprises, in 5′ to 3′ direction, exon 1, or a portion thereof of a RALGPS1 gene, fused to exon 3, or a portion thereof, and exons 4-11 of an ABL1 gene;
optionally, wherein the cancer is melanoma, and optionally wherein the melanoma is unknown primary melanoma.

Exemplary Embodiment 97: The method of any one of embodiments 1-14, 39-59, 62-80, 82-83, and 96, wherein the ABL1 fusion polypeptide encoded by the ABL1 fusion nucleic acid molecule comprises a fusion between an ABL1 polypeptide, or a portion thereof, fused to a RALGPS1 polypeptide, or a portion thereof, and wherein the ABL1 fusion polypeptide:
(a) comprises, in the N- to C-terminus direction, a RALGPS1 polypeptide, or a portion thereof, fused to an ABL1 polypeptide, or a portion thereof;
(b) comprises a fusion between an amino acid sequence encoded by exon 1, or a portion thereof, of a RALGPS1 gene fused to an amino acid sequence encoded by exon 3, or a portion thereof, of an ABL1 gene;
(c) results from a breakpoint within intron 1 of a RALGPS1 gene and/or a breakpoint within intron 2 of an ABL1 gene;
(d) results from a 5′ breakpoint within chromosomal coordinates chr9: 129694023-129694282 and/or a 3′ breakpoint within chromosomal coordinates chr9: 133730118-133730529; and/or
(e) comprises, in the N- to C-terminus direction, an amino acid sequence encoded by exon 1, or a portion thereof, of a RALGPS1 gene, fused to an amino acid sequence encoded by exon 3, or a portion thereof, and exons 4-11 of an ABL1 gene;
optionally, wherein the cancer is melanoma, and optionally wherein the melanoma is unknown primary melanoma.

Exemplary Embodiment 98: The method of any one of embodiments 1-80 and 82-83, wherein the ABL1 fusion nucleic acid molecule comprises a fusion between an ABL1 gene, or a portion thereof, fused to a RFFL gene, or a portion thereof, and wherein the ABL1 fusion nucleic acid molecule:
(a) comprises, in 5′ to 3′ direction a RFFL gene, or a portion thereof, fused to an ABL1 gene, or a portion thereof;
(b) comprises a fusion between exon 5, or a portion thereof, of a RFFL gene fused to exon 2, or a portion thereof, of an ABL1 gene;
(c) comprises or results from a 5′ breakpoint within chromosomal coordinates chr17: 33343379-33343419 and/or a 3′ breakpoint within chromosomal coordinates chr9: 133729485-133729525;
(d) comprises, in 5′ to 3′ direction, exons 1-4 and exon 5, or a portion thereof, of an RFFL gene fused to exon 2, or a portion thereof, and exons 3-11 of an ABL1 gene;
(e) comprises the nucleotide sequence of SEQ ID NO: 4, or a nucleotide sequence having at least about 70% homology thereto; and/or
(f) encodes an ABL1 fusion polypeptide comprising the amino acid sequence of SEQ ID NO: 25, or an amino acid sequence having at least about 70% homology thereto;
optionally, wherein the cancer is a soft tissue malignant peripheral nerve sheath tumor (MPNST).

Exemplary Embodiment 99: The method of any one of embodiments 1-14, 39-59, 62-80, 82-83, and 98, wherein the ABL1 fusion polypeptide encoded by the ABL1 fusion nucleic acid molecule comprises a fusion between an ABL1 polypeptide, or a portion thereof, fused to a RFFL polypeptide, or a portion thereof, and wherein the ABL1 fusion polypeptide:
(a) comprises, in the N- to C-terminus direction, a RFFL polypeptide, or a portion thereof, fused to an ABL1 polypeptide, or a portion thereof;
(b) comprises a fusion between an amino acid sequence encoded by exon 5, or a portion thereof, of a RFFL gene fused to an amino acid sequence encoded by exon 2, or a portion thereof, of an ABL1 gene;
(c) results from a 5′ breakpoint within chromosomal coordinates chr17: 33343379-33343419 and/or a 3′ breakpoint within chromosomal coordinates chr9: 133729485-133729525;
(d) comprises, in the N- to C-terminus direction, an amino acid sequence encoded by exons 1-4 and exon 5, or a portion thereof, of an RFFL gene, fused to an amino acid sequence encoded by exon 2, or a portion thereof, and exons 3-11 of an ABL1 gene;
(e) is encoded by the nucleotide sequence of SEQ ID NO: 4, or a nucleotide sequence having at least about 70% homology thereto; and/or
(f) comprises the amino acid sequence of SEQ ID NO: 25, or an amino acid sequence having at least about 70% homology thereto;
optionally, wherein the cancer is a soft tissue malignant peripheral nerve sheath tumor (MPNST).

Exemplary Embodiment 100: The method of any one of embodiments 1-80 and 82-83, wherein the ABL1 fusion nucleic acid molecule comprises a fusion between an ABL1 gene, or a portion thereof, fused to a SLC27A4 gene, or a portion thereof, and wherein the ABL1 fusion nucleic acid molecule:
(a) comprises, in 5′ to 3′ direction, a SLC27A4 gene, or a portion thereof, fused to an ABL1 gene, or a portion thereof;
(b) comprises a fusion between exon 12, or a portion thereof, of a SLC27A4 gene fused to exon 6, or a portion thereof, of an ABL1 gene;
(c) comprises or results from a breakpoint within intron 12 of a SLC27A4 gene and/or a breakpoint within intron 5 of an ABL1 gene;
(d) comprises or results from a 5′ breakpoint within chromosomal coordinates chr9: 131119603-131119801 and/or a 3′ breakpoint within chromosomal coordinates chr9: 133748211-133748501;
(e) comprises, in 5′ to 3′ direction, exons 1-11 and exon 12, or a portion thereof, of a SLC27A4 gene, fused to exon 6, or a portion thereof, and exons 7-11 of an ABL1 gene;
(f) comprises the nucleotide sequence of SEQ ID NO: 5, or a nucleotide sequence having at least about 70% homology thereto; and/or
(g) encodes an ABL1 fusion polypeptide comprising the amino acid sequence of SEQ ID NO: 26, or an amino acid sequence having at least about 70% homology thereto;
optionally, wherein the cancer is a testis germ cell tumor, and optionally wherein the testis germ cell tumor is a mixed testis germ cell tumor.

Exemplary Embodiment 101: The method of any one of embodiments 1-14, 39-59, 62-80, 82-83, and 100, wherein the ABL1 fusion polypeptide encoded by the ABL1 fusion nucleic acid molecule comprises a fusion between an ABL1 polypeptide, or a portion thereof, fused to a SLC27A4 polypeptide, or a portion thereof, and wherein the ABL1 fusion polypeptide:
(a) comprises, in the N- to C-terminus direction, a SLC27A4 polypeptide, or a portion thereof, fused to an ABL1 polypeptide, or a portion thereof;
(b) comprises a fusion between an amino acid sequence encoded by exon 12, or a portion thereof, of a SLC27A4 gene fused to an amino acid sequence encoded by exon 6, or a portion thereof, of an ABL1 gene;
(c) results from a breakpoint within intron 12 of a SLC27A4 gene and/or a breakpoint within intron 5 of an ABL1 gene;
(d) results from a 5′ breakpoint within chromosomal coordinates chr9: 131119603-131119801 and/or a 3′ breakpoint within chromosomal coordinates chr9: 133748211-133748501;
(e) comprises, in the N- to C-terminus direction, an amino acid sequence encoded by exons 1-11 and exon 12, or a portion thereof, of an SLC27A4 gene, fused to an amino acid sequence encoded by exon 6, or a portion thereof, and exons 7-11 of an ABL1 gene;
(f) is encoded by the nucleotide sequence of SEQ ID NO: 5, or a nucleotide sequence having at least about 70% homology thereto; and/or
(g) comprises the amino acid sequence of SEQ ID NO: 26, or an amino acid sequence having at least about 70% homology thereto;
optionally, wherein the cancer is a testis germ cell tumor, and optionally wherein the testis germ cell tumor is a mixed testis germ cell tumor.

Exemplary Embodiment 102: The method of any one of embodiments 1-80 and 82-83, wherein the ABL1 fusion nucleic acid molecule comprises a fusion between an ABL1 gene, or a portion thereof, fused to a TSC1 gene, or a portion thereof, and wherein the ABL1 fusion nucleic acid molecule:
(a) comprises, in 5′ to 3′ direction, a TSC1 gene, or a portion thereof, fused to an ABL1 gene, or a portion thereof;
(b) comprises a fusion between exon 19, or a portion thereof, of a TSC1 gene fused to exon 2, or a portion thereof, of an ABL1 gene;
(c) comprises or results from a 5′ breakpoint within chromosomal coordinates chr9:135,776,998-135,777,038 and/or a 3′ breakpoint within chromosomal coordinates chr9: 133729491-133729531; and/or
(d) comprises, in 5′ to 3′ direction, exons 1-18 and exon 19, or a portion thereof, of a TSC1 gene, fused to exon 2, or a portion thereof, and exons 3-11 of an ABL1 gene;
optionally, wherein the cancer is a soft tissue malignant peripheral nerve sheath tumor (MPNST).

Exemplary Embodiment 103: The method of any one of embodiments 1-14, 39-59, 62-80, 82-83, and 102, wherein the ABL1 fusion polypeptide encoded by the ABL1 fusion nucleic acid molecule comprises a fusion between an ABL1 polypeptide, or a portion thereof, fused to a TSC1 polypeptide, or a portion thereof, and wherein the ABL1 fusion polypeptide:
(a) comprises, in the N- to C-terminus direction, a TSC1 polypeptide, or a portion thereof, fused to an ABL1 polypeptide, or a portion thereof;
(b) comprises a fusion between an amino acid sequence encoded by exon 19, or a portion thereof, of a TSC1 gene fused to an amino acid sequence encoded by exon 2, or a portion thereof, of an ABL1 gene;
(c) results from a 5′ breakpoint within chromosomal coordinates chr9:135,776,998-135,777,038 and/or a 3′ breakpoint within chromosomal coordinates chr9: 133729491-133729531; and/or
(d) comprises, in 5′ to 3′ direction, an amino acid sequence encoded by exons 1-18 and exon 19, or a portion thereof, of a TSC1 gene, fused to an amino acid sequence encoded by exon 2, or a portion thereof, and exons 3-11 of an ABL1 gene;
optionally, wherein the cancer is a soft tissue malignant peripheral nerve sheath tumor (MPNST).

Exemplary Embodiment 104: The method of any one of embodiments 1-80 and 82-83, wherein the ABL1 fusion nucleic acid molecule comprises a fusion between an ABL1 gene, or a portion thereof, fused to a TSPAN18 gene, or a portion thereof, and wherein the ABL1 fusion nucleic acid molecule:
(a) comprises, in 5′ to 3′ direction, a TSPAN18 gene, or a portion thereof, fused to an ABL1 gene, or a portion thereof;
(b) comprises a fusion between exon 2, or a portion thereof, of a TSPAN18 gene fused to exon 2, or a portion thereof, of an ABL1 gene;
(c) comprises or results from a 5′ breakpoint within chromosomal coordinates chr11: 44881929-44881969 and/or a 3′ breakpoint within chromosomal coordinates chr9: 133729517-133729557; and/or
(d) comprises, in 5′ to 3′ direction, exon 1 and exon 2, or a portion thereof, of a TSPAN18 gene, fused to exon 2, or a portion thereof, and exons 3-11 of an ABL1 gene;
optionally, wherein the cancer is a soft tissue osteosarcoma, and optionally wherein the soft tissue osteosarcoma is an extraskeletal soft tissue osteosarcoma.

Exemplary Embodiment 105: The method of any one of embodiments 1-14, 39-59, 62-80, 82-83, and 104, wherein the ABL1 fusion polypeptide encoded by the ABL1 fusion nucleic acid molecule comprises a fusion between an ABL1 polypeptide, or a portion thereof, fused to a TSPAN18 polypeptide, or a portion thereof, and wherein the ABL1 fusion polypeptide:
(a) comprises, in the N- to C-terminus direction, a TSPAN18 polypeptide, or a portion thereof, fused to an ABL1 polypeptide, or a portion thereof;
(b) comprises a fusion between an amino acid sequence encoded by exon 2, or a portion thereof, of a TSPAN18 gene fused to an amino acid sequence encoded by exon 2, or a portion thereof, of an ABL1 gene;
(c) results from a 5′ breakpoint within chromosomal coordinates chr11: 44881929-44881969 and/or a 3′ breakpoint within chromosomal coordinates chr9: 133729517-133729557; and/or
(d) comprises, in the N- to C-terminus direction, an amino acid sequence encoded by exon 1 and exon 2, or a portion thereof, of a TSPAN18 gene, fused to an amino acid sequence encoded by exon 2, or a portion thereof, and exons 3-11 of an ABL1 gene;
optionally, wherein the cancer is a soft tissue osteosarcoma, and optionally wherein the soft tissue osteosarcoma is an extraskeletal soft tissue osteosarcoma.

Exemplary Embodiment 106: The method of any one of embodiments 1-80 and 82-83, wherein the ABL1 fusion nucleic acid molecule comprises a fusion between an ABL1 gene, or a portion thereof, fused to a ZNF804B gene, or a portion thereof, and wherein the ABL1 fusion nucleic acid molecule:
(a) comprises, in 5′ to 3′ direction a ZNF804B gene, or a portion thereof, fused to an ABL1 gene, or a portion thereof;
(b) comprises a fusion between exon 1, or a portion thereof, of a ZNF804B gene fused to exon 2, or a portion thereof, of an ABL1 gene;
(c) comprises or results from a breakpoint within intron 1 of a ZNF804B gene and/or a breakpoint within intron 1 of an ABL1 gene;
(d) comprises or results from a 5′ breakpoint within chromosomal coordinates chr7: 88521265-88521397 and/or a 3′ breakpoint within chromosomal coordinates chr9: 133729414-133729519; and/or
(e) comprises, in 5′ to 3′ direction, exon 1, or a portion thereof, of a ZNF804B gene, fused to exon 2, or a portion thereof, and exons 3-11 of an ABL1 gene;
optionally, wherein the cancer is a melanoma, optionally wherein the melanoma is a skin melanoma.

Exemplary Embodiment 107: The method of any one of embodiments 1-14, 39-59, 62-80, 82-83, and 106, wherein the ABL1 fusion polypeptide encoded by the ABL1 fusion nucleic acid molecule comprises a fusion between an ABL1 polypeptide, or a portion thereof, fused to a ZNF804B polypeptide, or a portion thereof, and wherein the ABL1 fusion polypeptide:
(a) comprises, in the N- to C-terminus direction, a ZNF804B polypeptide, or a portion thereof, fused to an ABL1 polypeptide, or a portion thereof;
(b) comprises a fusion between an amino acid sequence encoded by exon 1, or a portion thereof, of a ZNF804B gene fused to an amino acid sequence encoded by exon 2, or a portion thereof, of an ABL1 gene;
(c) results from a breakpoint within intron 1 of a ZNF804B gene and/or a breakpoint within intron 1 of an ABL1 gene;
(d) results from a 5′ breakpoint within chromosomal coordinates chr7: 88521265-88521397 and/or a 3′ breakpoint within chromosomal coordinates chr9: 133729414-133729519; and/or
(e) comprises, in the N- to C-terminus, an amino acid sequence encoded by exon 1, or a portion thereof, of a ZNF804B gene, fused to an amino acid sequence encoded by exon 2, or a portion thereof, and exons 3-11 of an ABL1 gene;
optionally, wherein the cancer is a melanoma, optionally wherein the melanoma is a skin melanoma.

Exemplary Embodiment 108: The method of any one of embodiments 1-79 and 81-83, wherein the ABL1 fusion nucleic acid molecule comprises a fusion between an ABL1 gene, or a portion thereof, fused to a second gene, or a portion thereof, wherein the ABL1 fusion nucleic acid molecule comprises or results from an ABL1 breakpoint within exon 1 of ABL1, and wherein the ABL1 fusion nucleic acid molecule:
(a) comprises, in 5′ to 3′ direction, the second gene, or a portion thereof, fused to exon 1 of an ABL1 gene, or a portion thereof;
(b) comprises a fusion between an exon or an intron, or a portion thereof, of the second gene fused to exon 1, or a portion thereof, of an ABL1 gene;
(c) comprises or results from a breakpoint within chromosomal coordinates chr9: 133589786-133589985; and/or
(d) comprises, in 5′ to 3′ direction, one or more exons, or portions thereof, of the second gene, fused to exon 1, or a portion thereof, and exons 2-11 of ABL1;
optionally wherein the cancer is a peritoneum adenocarcinoma.

Exemplary Embodiment 109: The method of any one of embodiments 1-14, 39-59, 62-79, 81-83 and 108, wherein the ABL1 fusion polypeptide encoded by the ABL1 fusion nucleic acid molecule comprises a fusion between an ABL1 polypeptide, or a portion thereof, fused to a polypeptide encoded by a second gene, or a portion thereof, wherein the ABL1 fusion polypeptide results from an ABL1 breakpoint within exon 1 of an ABL1 gene, and wherein the ABL1 fusion polypeptide:
(a) comprises, in the N- to C-terminus direction, an amino acid sequence encoded by the second gene, or a portion thereof, fused to an amino acid sequence encoded by exon 1 of an ABL1 gene, or a portion thereof;
(b) comprises a fusion between an amino acid sequence encoded by an exon, or a portion thereof, of the second gene fused to an amino acid sequence encoded by exon 1, or a portion thereof, of an ABL1 gene;
(c) results from a breakpoint within chromosomal coordinates chr9: 133589786-133589985; and/or
(d) comprises, in the N- to C-terminus direction, an amino acid sequence encoded by one or more exons, or portions thereof, of the second gene, fused to an amino acid sequence encoded by exon 1, or a portion thereof, and exons 2-11 of ABL1;
optionally wherein the cancer is a peritoneum adenocarcinoma.

Exemplary Embodiment 110: The method of embodiment 108, wherein the second gene is an EXOSC2 gene, and wherein the ABL1 fusion nucleic acid molecule:
(a) comprises a fusion between exon 9, or a portion thereof, of an EXOSC2 gene fused to exon 1, or a portion thereof, of an ABL1 gene;
(b) comprises or results from a breakpoint within exon 9 of an EXOSC2 gene and a breakpoint within exon 1 of an ABL1 gene;
(c) comprises or results from a 5′ breakpoint within chromosomal coordinates chr9: 133579154-133579531 and/or a 3′ breakpoint within chromosomal coordinates chr9: 133589786-133589985; and/or
(d) comprises, in 5′ to 3′ direction, exons 1-8 and exon 9, or a portion thereof, of an EXOSC2 gene, fused to exon 1, or a portion thereof, and exons 2-11 of an ABL1 gene;
optionally, wherein the cancer is a peritoneum adenocarcinoma.

Exemplary Embodiment 111: The method of embodiment 109, wherein the second gene is an EXOSC2 gene, and wherein the ABL1 fusion polypeptide:
(a) comprises a fusion between an amino acid sequence encoded by exon 9, or a portion thereof, of an EXOSC2 gene fused to an amino acid sequence encoded by exon 1, or a portion thereof, of an ABL1 gene;
(b) results from a breakpoint within exon 9 of an EXOSC2 gene and a breakpoint within exon 1 of an ABL1 gene;
(c) results from a 5′ breakpoint within chromosomal coordinates chr9: 133579154-133579531 and/or a 3′ breakpoint within chromosomal coordinates chr9: 133589786-133589985; and/or
(d) comprises, in the N- to C-terminus direction, an amino acid sequence encoded by exons 1-8 and exon 9, or a portion thereof, of an EXOSC2 gene, fused to an amino acid sequence encoded by exon 1, or a portion thereof, and exons 2-11 of an ABL1 gene;
optionally, wherein the cancer is a peritoneum adenocarcinoma.

Exemplary Embodiment 112: The method of any one of embodiments 1-79 and 81-83, wherein the ABL1 fusion nucleic acid molecule comprises a fusion between an ABL1 gene, or a portion thereof, fused to a second gene, or a portion thereof, wherein the ABL1 fusion nucleic acid molecule comprises or results from an ABL1 breakpoint within exon 2 of ABL1, and wherein the ABL1 fusion nucleic acid molecule:
(a) comprises, in 5′ to 3′ direction, the second gene, or a portion thereof, fused to exon 2, or a portion thereof, of an ABL1 gene;
(b) comprises a fusion between an exon or an intron, or a portion thereof, of the second gene fused to exon 2, or a portion thereof, of an ABL1 gene;
(c) comprises or results from a breakpoint within chromosomal coordinates chr9: 133729584-133729766; and/or
(d) comprises, in 5′ to 3′ direction, one or more exons, or portions thereof, of the second gene, fused to exon 2, or a portion thereof, and exons 3-11 of an ABL1 gene;
optionally wherein the cancer is a lung cancer, and optionally wherein the lung cancer is a lung adenocarcinoma.

Exemplary Embodiment 113: The method of any one of embodiments 1-14, 39-59, 62-79, 81-83 and 112, wherein the ABL1 fusion polypeptide encoded by the ABL1 fusion nucleic acid molecule comprises a fusion between an ABL1 polypeptide, or a portion thereof, fused to a polypeptide encoded by a second gene, or a portion thereof, wherein the ABL1 fusion polypeptide results from an ABL1 breakpoint within exon 2 of ABL1, and wherein the ABL1 fusion polypeptide: (a) comprises, in the N- to C-terminus direction, an amino acid sequence encoded by the second gene, or a portion thereof, fused to an amino acid sequence encoded by exon 2, or a portion thereof, of an ABL1 gene;
(b) comprises a fusion between an amino acid sequence encoded by an exon, or a portion thereof, of the second gene fused to an amino acid sequence encoded by exon 2, or a portion thereof, of an ABL1 gene;
(c) results from a breakpoint within chromosomal coordinates chr9: 133729584-133729766; and/or
(d) comprises, in the N- to C-terminus direction, an amino acid sequence encoded by one or more exons, or portions thereof, of the second gene, fused to an amino acid sequence encoded by exon 2, or a portion thereof, and exons 3-11 of an ABL1 gene;
optionally wherein the cancer is a lung cancer, and optionally wherein the lung cancer is a lung adenocarcinoma.

Exemplary Embodiment 114: The method of embodiment 112, wherein the second gene is an APOOL gene, and wherein the ABL1 fusion nucleic acid molecule:
(a) comprises or results from a breakpoint within intron 2 of an APOOL gene, and exon 2 of ABL1;
(b) comprises a fusion between exon 2, or a portion thereof, of an APOOL gene fused to exon 2, or a portion thereof, of an ABL1 gene;
(c) comprises or results from a 5′ breakpoint within chromosomal coordinates chrX: 84302705-84302904 and/or 3′ breakpoint within chromosomal coordinates chr9: 133729584-133729766; and/or
(d) comprises, in 5′ to 3′ direction, exon 1 and exon 2, or a portion thereof of an APOOL gene, fused to exon 2, or a portion thereof, and exons 3-11 of an ABL1 gene;
optionally, wherein the cancer is a lung cancer, and optionally wherein the lung cancer is a lung adenocarcinoma.

Exemplary Embodiment 115: The method of embodiment 113, wherein the second gene is an APOOL gene, and wherein the ABL1 fusion polypeptide:
(a) results from a breakpoint within intron 2 of an APOOL gene, and exon 2 of an ABL1 gene;
(b) comprises a fusion between an amino acid sequence encoded by exon 2, or a portion thereof, of an APOOL gene fused to an amino acid sequence encoded by exon 2, or a portion thereof, of an ABL1 gene;
(c) results from a 5′ breakpoint within chromosomal coordinates chrX: 84302705-84302904 and/or 3′ breakpoint within chromosomal coordinates chr9: 133729584-133729766; and/or
(d) comprises, in the N- to C-terminus direction, an amino acid sequence encoded by exon 1 and exon 2, or a portion thereof of an APOOL gene, fused to an amino acid sequence encoded by exon 2, or a portion thereof, and exons 3-11 of an ABL1 gene;
optionally, wherein the cancer is a lung cancer, and optionally wherein the lung cancer is a lung adenocarcinoma.

Exemplary Embodiment 116: The method of any one of embodiments 1-79 and 81-83, wherein the ABL1 fusion nucleic acid molecule comprises a fusion between an ABL1 gene, or a portion thereof, fused to a second gene, or a portion thereof, wherein the ABL1 fusion nucleic acid molecule comprises or results from an ABL1 breakpoint within exon 3 of ABL1, and wherein the ABL1 fusion nucleic acid molecule:
(a) comprises, in 5′ to 3′ direction, the second gene, or a portion thereof, fused to exon 3, or a portion thereof, of an ABL1 gene;
(b) comprises a fusion between an exon or an intron, or a portion thereof, of the second gene fused to exon 3, or a portion thereof, of an ABL1 gene;
(c) comprises or results from a breakpoint within chromosomal coordinates chr9: 133730205-133730245, chr9: 133730195-133730235, chr9: 133730203-133730243, chr9: 133730234-133730274 or chr9: 133730218-133730258; and/or
(d) comprises, in 5′ to 3′ direction, one or more exons, or portions thereof, of the second gene, fused to exon 3, or a portion thereof, and exons 4-11 of an ABL1 gene;
optionally wherein the cancer is a bone marrow leukemia, optionally wherein the bone marrow leukemia is a bone marrow leukemia lymphocytic acute (ALL), a bone marrow leukemia B cell acute (B-ALL), or a bone marrow leukemia T cell acute (T-ALL).

Exemplary Embodiment 117: The method of any one of embodiments 1-14, 39-59, 62-79, 81-83 and 116, wherein the ABL1 fusion polypeptide encoded by the ABL1 fusion nucleic acid molecule comprises a fusion between an ABL1 polypeptide, or a portion thereof, fused to a polypeptide encoded by a second gene, or a portion thereof, wherein the ABL1 fusion polypeptide results from an ABL1 breakpoint within exon 3 of an ABL1 gene, and wherein the ABL1 fusion polypeptide:
(a) comprises, in the N- to C-terminus direction, an amino acid sequence encoded by the second gene, or a portion thereof, fused to an amino acid sequence encoded by exon 3, or a portion thereof, of an ABL1 gene;
(b) comprises a fusion between an amino acid sequence encoded by an exon, or a portion thereof, of the second gene fused to an amino acid sequence encoded by exon 3, or a portion thereof, of an ABL1 gene;
(c) results from a breakpoint within chromosomal coordinates chr9: 133730205-133730245, chr9: 133730195-133730235, chr9: 133730203-133730243, chr9: 133730234-133730274 or chr9: 133730218-133730258; and/or
(d) comprises, in the N- to C-terminus direction, an amino acid sequence encoded by one or more exons, or portions thereof, of the second gene, fused to an amino acid sequence encoded by exon 3, or a portion thereof, and exons 4-11 of an ABL1 gene;
optionally wherein the cancer is a bone marrow leukemia, optionally wherein the bone marrow leukemia is a bone marrow leukemia lymphocytic acute (ALL), a bone marrow leukemia B cell acute (B-ALL), or a bone marrow leukemia T cell acute (T-ALL).

Exemplary Embodiment 118: The method of embodiment 116, wherein the second gene is a NUP214 gene, and wherein the ABL1 fusion nucleic acid molecule:
(a) comprises a fusion between exon 34, or a portion thereof, of a NUP214 gene fused to exon 3, or a portion thereof, of an ABL1 gene;
(b) comprises or results from a breakpoint within intron 34 of a NUP214 gene and a breakpoint within exon 3 of an ABL1 gene;
(c) comprises or results from:
   a 5′ breakpoint within chromosomal coordinates chr9: 134106080-134106120 and/or a 3′ breakpoint within chromosomal coordinates chr9: 133730195-133730235,
   a 5′ breakpoint within chromosomal coordinates chr9: 134106030-134106070 and/or a 3′ breakpoint within chromosomal coordinates chr9: 133730218-133730258,
   a 5′ breakpoint within chromosomal coordinates chr9: 134106046-134106086 and/or a 3′ breakpoint within chromosomal coordinates chr9: 133730203-133730243, or
   a 5′ breakpoint within chromosomal coordinates chr9: 134106028-134106068 and/or a 3′ breakpoint within chromosomal coordinates chr9: 133730234-133730274; and/or
(d) comprises, in 5′ to 3′ direction, exons 1-33 and exon 34, or a portion thereof, of a NUP214 gene, fused to exon 3, or a portion thereof, and exons 4-11 of an ABL1 gene;
optionally, wherein the cancer is a bone marrow leukemia, and optionally wherein the bone marrow leukemia is a bone marrow leukemia B cell acute (B-ALL) or a bone marrow leukemia lymphocytic acute (ALL).

Exemplary Embodiment 119: The method of embodiment 116, wherein the second gene is a BMP2K gene, and wherein the ABL1 fusion nucleic acid molecule:
(a) comprises a fusion between exon 15, or a portion thereof, of a BMP2K gene fused to exon 3, or a portion thereof, of an ABL1 gene;
(b) comprises or results from a 5′ breakpoint within chromosomal coordinates chr4: 79800022-79800062 and/or a 3′ breakpoint within chromosomal coordinates chr9: 133730205-133730245; and/or
(c) comprises, in 5′ to 3′ direction, exons 1-14 and exon 15, or a portion thereof, of a BMP2K gene, fused to exon 3, or a portion thereof, and exons 4-11 of an ABL1 gene;
optionally, wherein the cancer is a bone marrow leukemia, and optionally wherein the bone marrow leukemia is a bone marrow leukemia T cell acute (T-ALL).

Exemplary Embodiment 120: The method of embodiment 116, wherein the second gene is a BCR gene, and wherein the ABL1 fusion nucleic acid molecule:
(a) comprises a fusion between exon 1, or a portion thereof, of a BCR gene fused to exon 3, or a portion thereof, of an ABL1 gene;
(b) comprises or results from a 5′ breakpoint within chromosomal coordinates chr22: 23524272-23524312 and/or a 3′ breakpoint within chromosomal coordinates chr9: 133730218-133730258; and/or
(c) comprises, in 5′ to 3′ direction, exon 1, or a portion thereof of a BCR gene, fused to exon 3, or a portion thereof, and exons 4-11 of an ABL1 gene;
optionally, wherein the cancer is a bone marrow leukemia, and optionally wherein the bone marrow leukemia is a bone marrow leukemia lymphocytic acute (ALL).

Exemplary Embodiment 121: The method of embodiment 117, wherein the second gene is a NUP214 gene, and wherein the ABL1 fusion polypeptide:
(a) comprises a fusion between an amino acid sequence encoded by exon 34, or a portion thereof, of a NUP214 gene fused to an amino acid sequence encoded by exon 3, or a portion thereof, of an ABL1 gene,
(b) results from a breakpoint within intron 34 of a NUP214 gene and a breakpoint within exon 3 of an ABL1 gene;
(c) results from:
   a 5′ breakpoint within chromosomal coordinates chr9: 134106080-134106120 and/or a 3′ breakpoint within chromosomal coordinates chr9: 133730195-133730235,
   a 5′ breakpoint within chromosomal coordinates chr9: 134106030-134106070 and/or a 3′ breakpoint within chromosomal coordinates chr9: 133730218-133730258,
   a 5′ breakpoint within chromosomal coordinates chr9: 134106046-134106086 and/or a 3′ breakpoint within chromosomal coordinates chr9: 133730203-133730243, or
   a 5′ breakpoint within chromosomal coordinates chr9: 134106028-134106068 and/or a 3′ breakpoint within chromosomal coordinates chr9: 133730234-133730274; and/or
(d) comprises, in the N- to C-terminus direction, an amino acid sequence encoded by exons 1-33 and exon 34, or a portion thereof, of a NUP214 gene, fused to an amino acid sequence encoded by exon 3, or a portion thereof, and exons 4-11 of an ABL1 gene;
optionally, wherein the cancer is a bone marrow leukemia, and optionally wherein the bone marrow leukemia is a bone marrow leukemia B cell acute (B-ALL) or a bone marrow leukemia lymphocytic acute (ALL).

Exemplary Embodiment 122: The method of embodiment 117, wherein the second gene is a BMP2K gene, and wherein the ABL1 fusion polypeptide:
(a) comprises a fusion between an amino acid sequence encoded by exon 15, or a portion thereof, of a BMP2K gene fused to an amino acid sequence encoded by exon 3, or a portion thereof, of an ABL1 gene;
(b) results from a 5′ breakpoint within chromosomal coordinates chr4: 79800022-79800062 and/or a 3′ breakpoint within chromosomal coordinates chr9: 133730205-133730245; and/or
(c) comprises, in the N- to C-terminus direction, an amino acid sequence encoded by exons 1-14 and exon 15, or a portion thereof, of a BMP2K gene, fused to an amino acid sequence encoded by exon 3, or a portion thereof, and exons 4-11 of an ABL1 gene;
optionally, wherein the cancer is a bone marrow leukemia, and optionally wherein the bone marrow leukemia is a bone marrow leukemia T cell acute (T-ALL).

Exemplary Embodiment 123: The method of embodiment 117, wherein the second gene is a BCR gene, and wherein the ABL1 fusion polypeptide:
(a) comprises a fusion between an amino acid sequence encoded by exon 1, or a portion thereof, of a BCR gene fused to an amino acid sequence encoded by exon 3, or a portion thereof, of an ABL1 gene;
(b) results from a 5′ breakpoint within chromosomal coordinates chr22: 23524272-23524312 and/or a 3′ breakpoint within chromosomal coordinates chr9: 133730218-133730258; and/or
(c) comprises, in the N- to C-terminus direction, an amino acid sequence encoded by exon 1, or a portion thereof, of a BCR gene, fused to an amino acid sequence encoded by exon 3, or a portion thereof, and exons 4-11 of an ABL1 gene;
optionally, wherein the cancer is a bone marrow leukemia, and optionally wherein the bone marrow leukemia is a bone marrow leukemia lymphocytic acute (ALL).

Exemplary Embodiment 124: The method of any one of embodiments 1-79 and 81-83, wherein the ABL1 fusion nucleic acid molecule comprises a fusion between an ABL1 gene, or a portion thereof, fused to a second gene, or a portion thereof, wherein the ABL1 fusion nucleic acid molecule comprises or results from an ABL1 breakpoint within exon 4 of ABL1, and wherein the ABL1 fusion nucleic acid molecule:
(a) comprises, in 5′ to 3′ direction, the second gene, or a portion thereof, fused to exon 4, or a portion thereof, of an ABL1 gene;
(b) comprises a fusion between an exon or an intron, or a portion thereof, of the second gene fused to exon 4, or a portion thereof, of an ABL1 gene;
(c) comprises or results from a breakpoint within chromosomal coordinates chr9: 133738173-133738213, chr9: 133738222-133738262, or chr9: 133738214-133738254; and/or
(d) comprises, in 5′ to 3′ direction, one or more exons, or portions thereof, of the second gene, fused to exon 4, or a portion thereof, and exons 5-11 of an ABL1 gene;
optionally wherein the cancer is a leukemia, optionally wherein the leukemia is a bone marrow leukemia B cell acute (B-ALL), a B-lymphoblastic leukemia-lymphoma (B-ALL), or a bone marrow leukemia lymphocytic chronic (CLL).

Exemplary Embodiment 125: The method of any one of embodiments 1-14, 39-59, 62-79, 81-83 and 124, wherein the ABL1 fusion polypeptide encoded by the ABL1 fusion nucleic acid molecule comprises a fusion between an ABL1 polypeptide, or a portion thereof, fused to a polypeptide encoded by a second gene, or a portion thereof, wherein the ABL1 fusion polypeptide comprises or results from an ABL1 breakpoint within exon 4 of ABL1, and wherein the ABL1 fusion polypeptide:
(a) comprises, in the N- to C-terminus direction, an amino acid sequence encoded by the second gene, or a portion thereof, fused to an amino acid sequence encoded by exon 4, or a portion thereof, of an ABL1 gene;
(b) comprises a fusion between an amino acid sequence encoded by an exon, or a portion thereof, of the second gene fused to an amino acid sequence encoded by exon 4, or a portion thereof, of an ABL1 gene;
(c) results from a breakpoint within chromosomal coordinates chr9: 133738173-133738213, chr9: 133738222-133738262, or chr9: 133738214-133738254; and/or
(d) comprises, in the N- to C-terminus direction, an amino acid sequence encoded by one or more exons, or portions thereof, of the second gene, fused to an amino acid sequence encoded by exon 4, or a portion thereof, and exons 5-11 of ABL1;
optionally wherein the cancer is a leukemia, optionally wherein the leukemia is a bone marrow leukemia B cell acute (B-ALL), a B-lymphoblastic leukemia-lymphoma (B-ALL), or a bone marrow leukemia lymphocytic chronic (CLL).

Exemplary Embodiment 126: The method of embodiment 124, wherein the second gene is an RCSD1 gene, and wherein the ABL1 fusion nucleic acid molecule:
(a) comprises a fusion between exon 4, or a portion thereof, of an RCSD1 gene fused to exon 4, or a portion thereof, of an ABL1 gene;
(b) comprises or results from a 5′ breakpoint within chromosomal coordinates chr1: 167654668-167654708 and/or a 3′ breakpoint within chromosomal coordinates chr9: 133738173-133738213;
(c) comprises, in 5′ to 3′ direction, exons 1-3 and exon 4, or a portion thereof, of an RCSD1 gene, fused to exon 4, or a portion thereof, and exons 5-11 of an ABL1 gene;
(d) comprises the nucleotide sequence of SEQ ID NO: 21, or a nucleotide sequence having at least about 70% homology thereto; and/or
(e) encodes an ABL1 fusion polypeptide comprising the amino acid sequence of SEQ ID NO: 42, or an amino acid sequence having at least about 70% homology thereto;
optionally, wherein the cancer is a bone marrow leukemia, and optionally wherein the bone marrow leukemia is a bone marrow leukemia lymphocytic chronic (CLL).

Exemplary Embodiment 127: The method of embodiment 124, wherein the second gene is an SNX2 gene, and wherein the ABL1 fusion nucleic acid molecule:
(a) comprises a fusion between exon 3, or a portion thereof, of an SNX2 gene fused to exon 4, or a portion thereof, of an ABL1 gene;
(b) comprises or results from:
   a 5′ breakpoint within chromosomal coordinates chr5: 122135451-122135491 and/or a 3′ breakpoint within chromosomal coordinates chr9: 133738222-133738262, or
   a 5′ breakpoint within chromosomal coordinates chr5: 122135445-122135485 and/or a 3′ breakpoint within chromosomal coordinates chr9: 133738214-133738254; and/or
(c) comprises, in 5′ to 3′ direction, exons 1-2 and exon 3, or a portion thereof of an SNX2 gene, fused to exon 4, or a portion thereof, and exons 5-11 of an ABL1 gene;
   optionally wherein the cancer is a leukemia, optionally wherein the leukemia is a bone marrow leukemia B cell acute (B-ALL) or a B-lymphoblastic leukemia-lymphoma (B-ALL).

Exemplary Embodiment 128: The method of embodiment 125, wherein the second gene is an RCSD1 gene, and wherein the ABL1 fusion polypeptide:
(a) comprises a fusion between an amino acid sequence encoded by exon 4, or a portion thereof, of an RCSD1 gene fused to an amino acid sequence encoded by exon 4, or a portion thereof, of an ABL1 gene;
(b) results from a 5′ breakpoint within chromosomal coordinates chr1: 167654668-167654708 and/or a 3′ breakpoint within chromosomal coordinates chr9: 133738173-133738213;
(c) comprises, in the N- to C-terminus direction, an amino acid sequence encoded by exons 1-3 and exon 4, or a portion thereof, of an RCSD1 gene, fused to an amino acid sequence encoded by exon 4, or a portion thereof, and exons 5-11 of an ABL1 gene;
(d) is encoded by the nucleotide sequence of SEQ ID NO: 21, or a nucleotide sequence having at least about 70% homology thereto; and/or
(e) comprises the amino acid sequence of SEQ ID NO: 42, or an amino acid sequence having at least about 70% homology thereto;
optionally, wherein the cancer is a bone marrow leukemia, and optionally wherein the bone marrow leukemia is a bone marrow leukemia lymphocytic chronic (CLL).

Exemplary Embodiment 129: The method of embodiment 125, wherein the second gene is an SNX2 gene, and wherein the ABL1 fusion polypeptide:
(a) comprises a fusion between an amino acid sequence encoded by exon 3, or a portion thereof, of an SNX2 gene fused to an amino acid sequence encoded by exon 4, or a portion thereof, of an ABL1 gene;
(b) results from:
   a 5′ breakpoint within chromosomal coordinates chr5: 122135451-122135491 and/or a 3′ breakpoint within chromosomal coordinates chr9: 133738222-133738262, or
   a 5′ breakpoint within chromosomal coordinates chr5: 122135445-122135485 and/or a 3′ breakpoint within chromosomal coordinates chr9: 133738214-133738254; and/or
(c) comprises, in the N- to C-terminus direction, an amino acid sequence encoded by exons 1-2 and exon 3, or a portion thereof, of an SNX2 gene, fused to an amino acid sequence encoded by exon 4, or a portion thereof, and exons 5-11 of an ABL1 gene;
optionally wherein the cancer is a leukemia, optionally wherein the leukemia is a bone marrow leukemia B cell acute (B-ALL) or a B-lymphoblastic leukemia-lymphoma (B-ALL).

Exemplary Embodiment 130: The method of any one of embodiments 1-79 and 81-83, wherein the ABL1 fusion nucleic acid molecule comprises a fusion between an ABL1 gene, or a portion thereof, fused to a second gene, or a portion thereof, wherein the ABL1 fusion nucleic acid molecule comprises or results from an ABL1 breakpoint within exon 10 of ABL1, and wherein the ABL1 fusion nucleic acid molecule:
(a) comprises, in 5′ to 3′ direction, exon 10, or a portion thereof, of an ABL1 gene fused to the second gene, or a portion thereof;
(b) comprises a fusion between an exon or an intron, or a portion thereof, of the second gene fused to exon 10, or a portion thereof, of an ABL1 gene;
(c) comprises or results from a breakpoint within chromosomal coordinates chr9: 133755953-133755993; and/or
(d) comprises, in 5′ to 3′ direction, exons 1-9 and exon 10, or a portion thereof, of an ABL1 gene fused to one or more exons, or portions thereof, of the second gene;
optionally wherein the cancer is soft tissue sarcoma, and optionally wherein the soft tissue sarcoma is not otherwise specified (NOS).

Exemplary Embodiment 131: The method of any one of embodiments 1-14, 39-59, 62-79, 81-83 and 130, wherein the ABL1 fusion polypeptide encoded by the ABL1 fusion nucleic acid molecule comprises a fusion between an ABL1 polypeptide, or a portion thereof, fused to a polypeptide encoded by a second gene, or a portion thereof, wherein the ABL1 fusion polypeptide results from an ABL1 breakpoint within exon 10 of an ABL1 gene, and wherein the ABL1 fusion polypeptide:
(a) comprises, in the N- to C-terminus direction, an amino acid sequence encoded by exon 10, or a portion thereof, of an ABL1 gene fused to an amino acid sequence encoded by the second gene, or a portion thereof;
(b) comprises a fusion between an amino acid sequence encoded by an exon, or a portion thereof, of the second gene fused to an amino acid sequence encoded by exon 10, or a portion thereof, of an ABL1 gene;
(c) results from a breakpoint within chromosomal coordinates chr9: 133755953-133755993; and/or
(d) comprises, in the N- to C-terminus direction, an amino acid sequence encoded by exons 1-9 and exon 10, or a portion thereof, of an ABL1 gene fused to an amino acid sequence encoded by one or more exons, or portions thereof, of the second gene;
optionally wherein the cancer is soft tissue sarcoma, and optionally wherein the soft tissue sarcoma is not otherwise specified (NOS).

Exemplary Embodiment 132: The method of embodiment 130, wherein the second gene is a DNAH2 gene, and wherein the ABL1 fusion nucleic acid molecule:
(a) comprises a fusion between exon 10, or a portion thereof, of an ABL1 gene fused to exon 4, or a portion thereof, of a DNAH2 gene;
(b) comprises or results from a 5′ breakpoint within chromosomal coordinates chr9: 133755953-133755993 and/or a 3′ breakpoint within chromosomal coordinates chr17: 7636399-7636439; and/or
(c) comprises, in 5′ to 3′ direction, exons 1-9 and exon 10, or a portion thereof, of an ABL1 gene fused to exon 4, or a portion thereof, and exons 5-85 of a DNAH2 gene;
optionally, wherein the cancer is a soft tissue sarcoma, and optionally wherein the soft tissue sarcoma is not otherwise specified (NOS).

Exemplary Embodiment 133: The method of embodiment 131, wherein the second gene is a DNAH2 gene, and wherein the ABL1 fusion polypeptide:
(a) comprises a fusion between an amino acid sequence encoded by exon 10, or a portion thereof, of an ABL1 gene fused to an amino acid sequence encoded by exon 4, or a portion thereof, of a DNAH2 gene;
(b) results from a 5′ breakpoint within chromosomal coordinates chr9: 133755953-133755993 and/or a 3′ breakpoint within chromosomal coordinates chr17: 7636399-7636439; and/or
(c) comprises, in the N- to C-terminus direction, an amino acid sequence encoded by exons 1-9 and exon 10, or a portion thereof, of an ABL1 gene fused to an amino acid sequence encoded by exon 4, or a portion thereof, and exons 5-85 of a DNAH2 gene;
optionally, wherein the cancer is a soft tissue sarcoma, and optionally wherein the soft tissue sarcoma is not otherwise specified (NOS).

Exemplary Embodiment 134: The method of any one of embodiments 1-79 and 81-83, wherein the ABL1 fusion nucleic acid molecule comprises a fusion between an ABL1 gene, or a portion thereof, fused to a second gene, or a portion thereof, wherein the ABL1 fusion nucleic acid molecule comprises or results from an ABL1 breakpoint within intron 4 of an ABL1 gene, and wherein the ABL1 fusion nucleic acid molecule:
(a) comprises, in 5′ to 3′ direction, the second gene, or a portion thereof, fused to exon 5, or a portion thereof, of an ABL1 gene;
(b) comprises a fusion between an exon or an intron of the second gene, or a portion thereof, fused to intron 4, or a portion thereof, of an ABL1 gene;
(c) comprises or results from a breakpoint within chromosomal coordinates chr9: 133747364-133747762; and/or
(d) comprises, in 5′ to 3′ direction, one or more exons, or portions thereof, of the second gene, fused to exon 5, or a portion thereof, and exons 6-11 of an ABL1 gene;
optionally wherein the cancer is an ovary epithelial carcinoma, and optionally wherein the ovary epithelial carcinoma is not otherwise specified (NOS).

Exemplary Embodiment 135: The method of any one of embodiments 1-14, 39-59, 62-79, 81-83 and 134, wherein the ABL1 fusion polypeptide encoded by the ABL1 fusion nucleic acid molecule comprises a fusion between an ABL1 polypeptide, or a portion thereof, fused to a polypeptide encoded by a second gene, or a portion thereof, wherein the ABL1 fusion polypeptide results from an ABL1 breakpoint within intron 4 of an ABL1 gene, and wherein the ABL1 fusion polypeptide:
(a) comprises, in the N- to C-terminus direction, an amino acid sequence encoded by the second gene, or a portion thereof, fused to an amino acid sequence encoded by exon 5, or a portion thereof, of an ABL1 gene;
(b) comprises a fusion between an amino acid sequence encoded by an exon of the second gene, or a portion thereof, fused to an amino acid sequence encoded by exon 5, or a portion thereof, of an ABL1 gene;
(c) results from a breakpoint within chromosomal coordinates chr9: 133747364-133747762; and/or
(d) comprises, in the N- to C-terminus direction, an amino acid sequence encoded by one or more exons, or portions thereof, of the second gene, fused to an amino acid sequence encoded by exon 5, or a portion thereof, and exons 6-11 of an ABL1 gene;
optionally wherein the cancer is an ovary epithelial carcinoma, and optionally wherein the ovary epithelial carcinoma is not otherwise specified (NOS).

Exemplary Embodiment 136: The method of embodiment 134, wherein the second gene is a MED27 gene, and wherein the ABL1 fusion nucleic acid molecule:
(a) comprises a fusion between exon 4, or a portion thereof, of a MED27 gene fused to exon 5, or a portion thereof, of an ABL1 gene;
(b) comprises or results from a breakpoint within intron 4 of a MED27 gene and a breakpoint within intron 4 of an ABL1 gene;
(c) comprises or results from a 5′ breakpoint within chromosomal coordinates chr9: 134774423-134776006 and/or a 3′ breakpoint within chromosomal coordinates chr9: 133747364-133747762;
(d) comprises, in 5′ to 3′ direction, exons 1-3 and exon 4, or a portion thereof of a MED27 gene, fused to exon 5, or a portion thereof, and exons 6-11 of an ABL1gene;
(e) comprises the nucleotide sequence of SEQ ID NO: 3, or a nucleotide sequence having at least about 70% homology thereto; and/or
(f) encodes an ABL1 fusion polypeptide comprising the amino acid sequence of SEQ ID NO: 24, or an amino acid sequence having at least about 70% homology thereto;
optionally, wherein the cancer is an ovary epithelial carcinoma, and optionally wherein the ovary epithelial carcinoma is not otherwise specified (NOS).

Exemplary Embodiment 137: The method of embodiment 135, wherein the second gene is a MED27 gene, and wherein the ABL1 fusion polypeptide:
(a) comprises a fusion between an amino acid sequence encoded by exon 4, or a portion thereof, of a MED27 gene fused to an amino acid sequence encoded by exon 5, or a portion thereof, of an ABL1 gene;
(b) results from a breakpoint within intron 4 of a MED27 gene and a breakpoint within intron 4 of an ABL1 gene;
(c) results from a 5′ breakpoint within chromosomal coordinates chr9: 134774423-134776006 and/or a 3′ breakpoint within chromosomal coordinates chr9: 133747364-133747762;
(d) comprises, in the N- to C-terminus direction, an amino acid sequence encoded by exons 1-3 and exon 4, or a portion thereof, of a MED27 gene, fused to an amino acid sequence encoded by exon 5, or a portion thereof, and exons 6-11 of an ABL1 gene;
(e) is encoded by the nucleotide sequence of SEQ ID NO: 3, or a nucleotide sequence having at least about 70% homology thereto; and/or
(f) comprises the amino acid sequence of SEQ ID NO: 24, or an amino acid sequence having at least about 70% homology thereto;
optionally, wherein the cancer is an ovary epithelial carcinoma, and optionally wherein the ovary epithelial carcinoma is not otherwise specified (NOS).

Exemplary Embodiment 138: The method of any one of embodiments 1-79 and 81-83, wherein the ABL1 fusion nucleic acid molecule comprises a fusion between an ABL1 gene, or a portion thereof, fused to a second gene, or a portion thereof, wherein the ABL1 fusion nucleic acid molecule comprises or results from an ABL1 breakpoint within intron 5 of an ABL1 gene, and wherein the ABL1 fusion nucleic acid molecule:
(a) comprises, in 5′ to 3′ direction, the second gene, or a portion thereof, fused to exon 6, or a portion thereof, of an ABL1 gene;
(b) comprises a fusion between an exon or an intron, or a portion thereof, of the second gene fused to intron 5, or a portion thereof, of an ABL1 gene;
(c) comprises or results from a breakpoint within chromosomal coordinates chr9: 133748211-133748501; and/or
(d) comprises, in 5′ to 3′ direction, one or more exons, or portions thereof, of the second gene, fused to exon 6, or a portion thereof, and exons 7-11 of an ABL1 gene;
optionally wherein the cancer is a testis germ cell tumor, and optionally wherein the testis germ cell tumor is a mixed testis germ cell tumor.

Exemplary Embodiment 139: The method of any one of embodiments 1-14, 39-59, 62-79, 81-83 and 138, wherein the ABL1 fusion polypeptide encoded by the ABL1 fusion nucleic acid molecule comprises a fusion between an ABL1 polypeptide, or a portion thereof, fused to a polypeptide encoded by a second gene, or a portion thereof, wherein the ABL1 fusion polypeptide results from an ABL1 breakpoint within intron 5 of ABL1, and wherein the ABL1 fusion polypeptide:
(a) comprises, in the N- to C-terminus direction, an amino acid sequence encoded by the second gene, or a portion thereof, fused to an amino acid sequence encoded by exon 6, or a portion thereof, of an ABL1 gene;
(b) comprises a fusion between an amino acid sequence encoded by an exon, or a portion thereof, of the second gene fused to an amino acid sequence encoded by exon 6, or a portion thereof, of an ABL1 gene;
(c) results from a breakpoint within chromosomal coordinates chr9: 133748211-133748501;
(d) comprises, in the N- to C-terminus direction, an amino acid sequence encoded by one or more exons, or portions thereof, of the second gene, fused to an amino acid sequence encoded by exon 6, or a portion thereof, and exons 7-11 of an ABL1 gene;
optionally wherein the cancer is a testis germ cell tumor, and optionally wherein the testis germ cell tumor is a mixed testis germ cell tumor.

Exemplary Embodiment 140: The method of embodiment 138, wherein the second gene is an SLC27A4 gene, and wherein the ABL1 fusion nucleic acid molecule:
(a) comprises a fusion between exon 12, or a portion thereof, of a SLC27A4 gene fused to exon 6, or a portion thereof, of an ABL1 gene;
(b) comprises or results from a breakpoint within intron 12 of a SLC27A4 gene and a breakpoint within intron 5 of an ABL1 gene;
(c) comprises or results from a 5′ breakpoint within chromosomal coordinates chr9: 131119603-131119801 and/or a 3′ breakpoint within chromosomal coordinates chr9: 133748211-133748501;
(d) comprises, in 5′ to 3′ direction, exons 1-11 and exon 12, or a portion thereof, of an SLC27A4 gene, fused to exon 6, or a portion thereof, and exons 7-11 of an ABL1 gene;
(e) comprises the nucleotide sequence of SEQ ID NO: 5, or a nucleotide sequence having at least about 70% homology thereto; and/or
(f) encodes an ABL1 fusion polypeptide comprising the amino acid sequence of SEQ ID NO: 26, or an amino acid sequence having at least about 70% homology thereto;
optionally, wherein the cancer is a testis germ cell tumor, wherein the testis germ cell tumor is a mixed testis germ cell tumor.

Exemplary Embodiment 141: The method of embodiment 139, wherein the second gene is an SLC27A4 gene, and wherein the ABL1 fusion polypeptide:
(a) comprises a fusion between an amino acid sequence encoded by exon 12, or a portion thereof, of a SLC27A4 gene fused to an amino acid sequence encoded by exon 6, or a portion thereof, of an ABL1 gene;
(b) results from a breakpoint within intron 12 of a SLC27A4 gene and a breakpoint within intron 5 of an ABL1 gene;
(c) results from a 5′ breakpoint within chromosomal coordinates chr9: 131119603-131119801 and/or a 3′ breakpoint within chromosomal coordinates chr9: 133748211-133748501;
(d) comprises, in the N- to C-terminus direction, an amino acid sequence encoded by exons 1-11 and exon 12, or a portion thereof, of an SLC27A4 gene, fused to an amino acid sequence encoded by exon 6, or a portion thereof, and exons 7-11 of an ABL1 gene;
(e) is encoded by the nucleotide sequence of SEQ ID NO: 5, or a nucleotide sequence having at least about 70% homology thereto; and/or
(f) comprises the amino acid sequence of SEQ ID NO: 26, or an amino acid sequence having at least about 70% homology thereto;
optionally, wherein the cancer is a testis germ cell tumor, wherein the testis germ cell tumor is a mixed testis germ cell tumor.

Exemplary Embodiment 142: The method of any one of embodiments 1-79, wherein the ABL1 fusion nucleic acid molecule comprises a fusion between an ABL1 gene, or a portion thereof, fused to a BCR gene, or a portion thereof, wherein the cancer is lung adenocarcinoma, and wherein the ABL1 fusion nucleic acid molecule:
(a) comprises, in 5′ to 3′ direction, a BCR gene, or a portion thereof, fused to an ABL1 gene, or a portion thereof;
(b) comprises:
   a fusion between exon 12, or a portion thereof, of a BCR gene fused to exon 2, or a portion thereof, of an ABL1 gene,
   a fusion between exon 13, or a portion thereof, of a BCR gene fused to exon 2, or a portion thereof, of an ABL1 gene, or
   a fusion between exon 14, or a portion thereof, of a BCR gene fused to exon 2, or a portion thereof, of an ABL1 gene;
(c) comprises or results from a breakpoint within intron 12, intron 13 or intron 14 of a BCR gene, and/or a breakpoint within intron 1 of an ABL1 gene;
(d) comprises or results from:
   a 5′ breakpoint within chromosomal coordinates chr22: 23632033-23632321 and/or a 3′ breakpoint within chromosomal coordinates chr9: 133591666-133591902,
   a 5′ breakpoint within chromosomal coordinates chr22: 23634537-23634672 and/or a 3′ breakpoint within chromosomal coordinates chr9: 133672815-133673059,
   a 5′ breakpoint within chromosomal coordinates chr22: 23634041-23634323 and/or a 3′ breakpoint within chromosomal coordinates chr9: 133708182-133708567,
   a 5′ breakpoint within chromosomal coordinates chr22: 23632683-23632948 and/or a 3′ breakpoint within chromosomal coordinates chr9: 133613427-133613626, or
   a 5′ breakpoint within chromosomal coordinates chr22: 23631693-23632188 and/or a 3′ breakpoint within chromosomal coordinates chr9: 133607683-133608245;
(e) comprises, in 5′ to 3′ direction:
   exons 1-11 and exon 12, or a portion thereof, of a BCR gene fused to exon 2, or a portion thereof, and exons 3-11 of an ABL1 gene,
   exons 1-12 and exon 13, or a portion thereof, of a BCR gene fused to exon 2, or a portion thereof, and exons 3-11 of an ABL1 gene, or
   exons 1-13 and exon 14, or a portion thereof, of a BCR gene, fused to exon 2, or a portion thereof, and exons 3-11 of an ABL1 gene;
(f) comprises the nucleotide sequence of any of SEQ ID NOs: 6-10, or a nucleotide sequence having at least about 70% homology thereto; and/or
(g) encodes an ABL1 fusion polypeptide comprising the amino acid sequence of any of SEQ ID NOS: 27-31, or an amino acid sequence having at least about 70% homology thereto.

Exemplary Embodiment 143: The method of any one of embodiments 1-14, 39-59, 62-79, and 142, wherein the ABL1 fusion polypeptide encoded by the ABL1 fusion nucleic acid molecule comprises a fusion between an ABL1 polypeptide, or a portion thereof, fused to a BCR polypeptide, or a portion thereof, wherein the cancer is lung adenocarcinoma, and wherein the ABL1 fusion polypeptide:
(a) comprises, in the N- to C-terminus direction, a BCR polypeptide, or a portion thereof, fused to an ABL1 polypeptide, or a portion thereof;
(b) comprises:
   a fusion between an amino acid sequence encoded by exon 12, or a portion thereof, of a BCR gene fused to an amino acid sequence encoded by exon 2, or a portion thereof, of an ABL1 gene,
   a fusion between an amino acid sequence encoded by exon 13, or a portion thereof, of a BCR gene fused to an amino acid sequence encoded by exon 2, or a portion thereof, of an ABL1 gene, or
   a fusion between an amino acid sequence encoded by exon 14, or a portion thereof, of a BCR gene fused to an amino acid sequence encoded by exon 2, or a portion thereof, of an ABL1 gene;
(c) results from a breakpoint within intron 12, intron 13 or intron 14 of a BCR gene, and/or a breakpoint within intron 1 of an ABL1 gene;
(d) results from:
   a 5′ breakpoint within chromosomal coordinates chr22: 23632033-23632321 and/or a 3′ breakpoint within chromosomal coordinates chr9: 133591666-133591902,
   a 5′ breakpoint within chromosomal coordinates chr22: 23634537-23634672 and/or a 3′ breakpoint within chromosomal coordinates chr9: 133672815-133673059,
   a 5′ breakpoint within chromosomal coordinates chr22: 23634041-23634323 and/or a 3′ breakpoint within chromosomal coordinates chr9: 133708182-133708567,
   a 5′ breakpoint within chromosomal coordinates chr22: 23632683-23632948 and/or a 3′ breakpoint within chromosomal coordinates chr9: 133613427-133613626, or
   a 5′ breakpoint within chromosomal coordinates chr22: 23631693-23632188 and/or a 3′ breakpoint within chromosomal coordinates chr9: 133607683-133608245;
(e) comprises, in the N- to C-terminus direction:
   an amino acid sequence encoded by exons 1-11 and exon 12, or a portion thereof, of a BCR gene fused to an amino acid sequence encoded by exon 2, or a portion thereof, and exons 3-11 of an ABL1 gene,
   an amino acid sequence encoded by exons 1-12 and exon 13, or a portion thereof, of a BCR gene fused to an amino acid sequence encoded by exon 2, or a portion thereof, and exons 3-11 of an ABL1 gene, or
   an amino acid sequence encoded by exons 1-13 and exon 14, or a portion thereof, of a BCR gene, fused to an amino acid sequence encoded by exon 2, or a portion thereof, and exons 3-11 of an ABL1 gene;
(f) is encoded by the nucleotide sequence of any of SEQ ID NOs: 6-10, or a nucleotide sequence having at least about 70% homology thereto; and/or
(g) comprises the amino acid sequence of any of SEQ ID NOs: 27-31, or an amino acid sequence having at least about 70% homology thereto.

Exemplary Embodiment 144: The method of any one of embodiments 1-79, wherein the ABL1 fusion nucleic acid molecule comprises a fusion between an ABL1 gene, or a portion thereof, fused to a BCR gene, or a portion thereof, wherein the cancer is head and neck squamous cell carcinoma (HNSCC), and wherein the ABL1 fusion nucleic acid molecule:
(a) comprises, in 5′ to 3′ direction, a BCR gene, or a portion thereof, fused to an ABL1 gene, or a portion thereof;
(b) comprises a fusion between exon 13 or a portion thereof, or exon 14 or a portion thereof, of a BCR gene fused to exon 2, or a portion thereof, of an ABL1 gene;
(c) comprises or results from a breakpoint within intron 13 or intron 14 of a BCR gene and/or a breakpoint within intron 1 of an ABL1 gene;
(d) comprises or results from:
   a 5′ breakpoint within chromosomal coordinates chr22: 23634011-23634460 and/or a 3′ breakpoint within chromosomal coordinates chr9: 133645693-133646017,
   a 5′ breakpoint within chromosomal coordinates chr22: 23634211-23634632 and/or a 3′ breakpoint within chromosomal coordinates chr9: 133619101-133619395,
   a 5′ breakpoint within chromosomal coordinates chr22: 23631687-23632492 and/or a 3′ breakpoint within chromosomal coordinates chr9: 133604425-133604577, or
   a 5′ breakpoint within chromosomal coordinates chr22: 23632288-23632656 and/or a 3′ breakpoint within chromosomal coordinates chr9: 133663291-133663682;
(e) comprises, in 5′ to 3′ direction:
   exons 1-13 and exon 14, or a portion thereof, of a BCR gene, fused to exon 2, or a portion thereof, and exons 3-11 of an ABL1 gene, or
   exons 1-12 and exon 13, or a portion thereof, of a BCR gene, fused to exon 2, or a portion thereof, and exons 3-11 of ABL1;
(f) comprises the nucleotide sequence of any of SEQ ID NOs: 11-12, or a nucleotide sequence having at least about 70% homology thereto; and/or
(g) encodes an ABL1 fusion polypeptide comprising the amino acid sequence of any of SEQ ID NOS: 32-33, or an amino acid sequence having at least about 70% homology thereto.

Exemplary Embodiment 145: The method of any one of embodiments 1-14, 39-59, 62-79, and 144, wherein the ABL1 fusion polypeptide encoded by the ABL1 fusion nucleic acid molecule comprises a fusion between an ABL1 polypeptide, or a portion thereof, fused to a BCR polypeptide, or a portion thereof, wherein the cancer is head and neck squamous cell carcinoma (HNSCC), and wherein the ABL1 fusion polypeptide:
(a) comprises, in the N- to C-terminus direction, a BCR polypeptide, or a portion thereof, fused to an ABL1 polypeptide, or a portion thereof;
(b) comprises a fusion between an amino acid sequence encoded by exon 13 or a portion thereof, or exon 14 or a portion thereof, of a BCR gene fused to an amino acid sequence encoded by exon 2, or a portion thereof, of an ABL1 gene;
(c) results from a breakpoint within intron 13 or intron 14 of a BCR gene and/or a breakpoint within intron 1 of an ABL1 gene;
(d) results from:
   a 5′ breakpoint within chromosomal coordinates chr22: 23634011-23634460 and/or a 3′ breakpoint within chromosomal coordinates chr9: 133645693-133646017,
   a 5′ breakpoint within chromosomal coordinates chr22: 23634211-23634632 and/or a 3′ breakpoint within chromosomal coordinates chr9: 133619101-133619395,
   a 5′ breakpoint within chromosomal coordinates chr22: 23631687-23632492 and/or a 3′ breakpoint within chromosomal coordinates chr9: 133604425-133604577, or
   a 5′ breakpoint within chromosomal coordinates chr22: 23632288-23632656 and/or a 3′ breakpoint within chromosomal coordinates chr9: 133663291-133663682;
(e) comprises, in the N- to C-terminus direction:
   an amino acid sequence encoded by exons 1-13 and exon 14, or a portion thereof, of a BCR gene, fused to an amino acid sequence encoded by exon 2, or a portion thereof, and exons 3-11 of an ABL1 gene, or
   an amino acid sequence encoded by exons 1-12 and exon 13, or a portion thereof, of a BCR gene, fused to an amino acid sequence encoded by exon 2, or a portion thereof, and exons 3-11 of ABL1;
(f) is encoded by the nucleotide sequence of any of SEQ ID NOs: 11-12, or a nucleotide sequence having at least about 70% homology thereto; and/or
(g) comprises the amino acid sequence of any of SEQ ID NOs: 32-33, or an amino acid sequence having at least about 70% homology thereto.

Exemplary Embodiment 146: The method of any one of embodiments 1-79, wherein the ABL1 fusion nucleic acid molecule comprises a fusion between an ABL1 gene, or a portion thereof, fused to a BCR gene, or a portion thereof, wherein the cancer is bone sarcoma, and wherein the ABL1 fusion nucleic acid molecule:
(a) comprises, in 5′ to 3′ direction, a BCR gene, or a portion thereof, fused to an ABL1 gene, or a portion thereof;
(b) comprises a fusion between exon 13, or a portion thereof, of a BCR gene fused to exon 2, or a portion thereof, of an ABL1 gene;
(c) comprises or results from a breakpoint within intron 13 of a BCR gene and/or a breakpoint within intron 1 of an ABL1 gene;
(d) comprises or results from a 5′ breakpoint within chromosomal coordinates chr22: 23631722-23631762 and/or a 3′ breakpoint within chromosomal coordinates chr9: 133729484-133729524;
(e) comprises, in 5′ to 3′ direction, exons 1-12 and exon 13, or a portion thereof, of a BCR gene fused to exon 2, or a portion thereof, and exons 3-11 of an ABL1 gene;
(f) comprises the nucleotide sequence of SEQ ID NO: 13, or a nucleotide sequence having at least about 70% homology thereto; and/or
(g) encodes an ABL1 fusion polypeptide comprising the amino acid sequence of SEQ ID NO: 34, or an amino acid sequence having at least about 70% homology thereto;
optionally wherein the bone sarcoma is not otherwise specified (NOS).

Exemplary Embodiment 147: The method of any one of embodiments 1-14, 39-59, 62-79, and 146, wherein the ABL1 fusion polypeptide encoded by the ABL1 fusion nucleic acid molecule comprises a fusion between an ABL1 polypeptide, or a portion thereof, fused to a BCR polypeptide, or a portion thereof, wherein the cancer is bone sarcoma, and wherein the ABL1 fusion polypeptide:
(a) comprises, in the N- to C-terminus direction, a BCR polypeptide, or a portion thereof, fused to an ABL1 polypeptide, or a portion thereof;
(b) comprises a fusion between an amino acid sequence encoded by exon 13, or a portion thereof, of a BCR gene fused to an amino acid sequence encoded by exon 2, or a portion thereof, of an ABL1 gene;
(c) results from a breakpoint within intron 13 of a BCR gene and/or a breakpoint within intron 1 of an ABL1 gene;
(d) results from a 5′ breakpoint within chromosomal coordinates chr22: 23631722-23631762 and/or a 3′ breakpoint within chromosomal coordinates chr9: 133729484-133729524;
(e) comprises, in the N- to C-terminus direction, an amino acid sequence encoded by exons 1-12 and exon 13, or a portion thereof, of a BCR gene, fused to an amino acid sequence encoded by exon 2, or a portion thereof, and exons 3-11 of an ABL1 gene;
(f) is encoded by the nucleotide sequence of SEQ ID NO: 13, or a nucleotide sequence having at least about 70% homology thereto; and/or
(g) comprises the amino acid sequence of SEQ ID NO: 34, or an amino acid sequence having at least about 70% homology thereto;
optionally wherein the bone sarcoma is not otherwise specified (NOS).

Exemplary Embodiment 148: The method of any one of embodiments 1-79, wherein the ABL1 fusion nucleic acid molecule comprises a fusion between an ABL1 gene, or a portion thereof, fused to a BCR gene, or a portion thereof, wherein the cancer is esophagus adenocarcinoma, and wherein the ABL1 fusion nucleic acid molecule:
(a) comprises, in 5′ to 3′ direction a BCR gene, or a portion thereof, fused to an ABL1 gene, or a portion thereof;
(b) comprises a fusion between exon 14, or a portion thereof, of a BCR gene fused to exon 2, or a portion thereof, of an ABL1 gene;
(c) comprises or results from a breakpoint within intron 14 of a BCR gene and/or a breakpoint within intron 1 of an ABL1 gene;
(d) comprises or results from a 5′ breakpoint within chromosomal coordinates chr22: 23633346-23634163 and/or a 3′ breakpoint within chromosomal coordinates chr9: 133609201-133609909;
(e) comprises, in 5′ to 3′ direction, exons 1-13 and exon 14, or a portion thereof, of a BCR gene, fused to exon 2, or a portion thereof, and exons 3-11 of an ABL1 gene;
(f) comprises the nucleotide sequence of SEQ ID NO: 14, or a nucleotide sequence having at least about 70% homology thereto; and/or
(g) encodes an ABL1 fusion polypeptide comprising the amino acid sequence of SEQ ID NO: 35, or an amino acid sequence having at least about 70% homology thereto.

Exemplary Embodiment 149: The method of any one of embodiments 1-14, 39-59, 62-79, and 148, wherein the ABL1 fusion polypeptide encoded by the ABL1 fusion nucleic acid molecule comprises a fusion between an ABL1 polypeptide, or a portion thereof, fused to a BCR polypeptide, or a portion thereof, wherein the cancer is esophagus adenocarcinoma, and wherein the ABL1 fusion polypeptide:
(a) comprises, in the N- to C-terminus direction, a BCR polypeptide, or a portion thereof, fused to an ABL1 polypeptide, or a portion thereof;
(b) comprises a fusion between an amino acid sequence encoded by exon 14, or a portion thereof, of a BCR gene fused to an amino acid sequence encoded by exon 2, or a portion thereof, of an ABL1 gene;
(c) results from a breakpoint within intron 14 of a BCR gene and/or a breakpoint within intron 1 of an ABL1 gene;
(d) results from a 5′ breakpoint within chromosomal coordinates chr22: 23633346-23634163 and/or a 3′ breakpoint within chromosomal coordinates chr9: 133609201-133609909;
(e) comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene;
(f) is encoded by the nucleotide sequence of SEQ ID NO: 14, or a nucleotide sequence having at least about 70% homology thereto; and/or
(g) comprises the amino acid sequence of SEQ ID NO: 35, or an amino acid sequence having at least about 70% homology thereto.

Exemplary Embodiment 150: The method of any one of embodiments 1-79, wherein the ABL1 fusion nucleic acid molecule comprises a fusion between an ABL1 gene, or a portion thereof, fused to a BCR gene, or a portion thereof, wherein the cancer is gastroesophageal junction adenocarcinoma, and wherein the ABL1 fusion nucleic acid molecule:
(a) comprises, in 5′ to 3′ direction, a BCR gene, or a portion thereof, fused to an ABL1 gene, or a portion thereof;
(b) comprises a fusion between exon 14, or a portion thereof, of a BCR gene fused to exon 2, or a portion thereof, of an ABL1 gene;
(c) comprises or results from a breakpoint within intron 14 of a BCR gene and/or a breakpoint within intron 1 of an ABL1 gene;
(d) comprises or results from a 5′ breakpoint within chromosomal coordinates chr22: 23633432-23633796 and/or a 3′ breakpoint within chromosomal coordinates chr9: 133623903-133624461;
(e) comprises, in 5′ to 3′ direction, exons 1-13 and exon 14, or a portion thereof, of a BCR gene fused to exon 2, or a portion thereof, and exons 3-11 of an ABL1 gene;
(f) comprises the nucleotide sequence of SEQ ID NO: 15, or a nucleotide sequence having at least about 70% homology thereto; and/or
(g) encodes an ABL1 fusion polypeptide comprising the amino acid sequence of SEQ ID NO: 36, or an amino acid sequence having at least about 70% homology thereto.

Exemplary Embodiment 151: The method of any one of embodiments 1-14, 39-59, 62-79, and 150, wherein the ABL1 fusion polypeptide encoded by the ABL1 fusion nucleic acid molecule comprises a fusion between an ABL1 polypeptide, or a portion thereof, fused to a BCR polypeptide, or a portion thereof, wherein the cancer is gastroesophageal junction adenocarcinoma, and wherein the ABL1 fusion polypeptide:

(a) comprises, in the N- to C-terminus direction, a BCR polypeptide, or a portion thereof, fused to an ABL1 polypeptide, or a portion thereof;
(b) comprises a fusion between an amino acid sequence encoded by exon 14, or a portion thereof, of a BCR gene fused to an amino acid sequence encoded by exon 2, or a portion thereof, of an ABL1 gene;
(c) results from a breakpoint within intron 14 of a BCR gene and/or a breakpoint within intron 1 of an ABL1 gene;
(d) results from a 5′ breakpoint within chromosomal coordinates chr22: 23633432-23633796 and/or a 3′ breakpoint within chromosomal coordinates chr9: 133623903-133624461;
(e) comprises, in the N- to C-terminus direction, an amino acid sequence encoded by exons 1-13 and exon 14, or a portion thereof, of a BCR gene fused to an amino acid sequence encoded by exon 2, or a portion thereof, and exons 3-11 of an ABL1 gene;
(f) is encoded by the nucleotide sequence of SEQ ID NO: 15, or a nucleotide sequence having at least about 70% homology thereto; and/or
(g) comprises the amino acid sequence of SEQ ID NO: 36, or an amino acid sequence having at least about 70% homology thereto.

Exemplary Embodiment 152: The method of any one of embodiments 1-79, wherein the ABL1 fusion nucleic acid molecule comprises a fusion between an ABL1 gene, or a portion thereof, fused to a BCR gene, or a portion thereof, wherein the cancer is liver cholangiocarcinoma, and wherein the ABL1 fusion nucleic acid molecule:
(a) comprises, in 5′ to 3′ direction, a BCR gene, or a portion thereof, fused to an ABL1 gene, or a portion thereof;
(b) comprises a fusion between exon 14, or a portion thereof, of a BCR gene fused to exon 2, or a portion thereof, of an ABL1 gene;
(c) comprises or results from a breakpoint within intron 14 of a BCR gene and/or a breakpoint within intron 1 of an ABL1 gene;
(d) comprises or results from a 5′ breakpoint within chromosomal coordinates chr22: 23633837-23634208 and/or a 3′ breakpoint within chromosomal coordinates chr9: 133665426-133665727;
(e) comprises, in 5′ to 3′ direction, exons 1-13 and exon 14, or a portion thereof, of a BCR gene, fused to exon 2, or a portion thereof, and exons 3-11 of an ABL1 gene;
(f) comprises the nucleotide sequence of SEQ ID NO: 16, or a nucleotide sequence having at least about 70% homology thereto; and/or
(g) encodes an ABL1 fusion polypeptide comprising the amino acid sequence of SEQ ID NO: 37, or an amino acid sequence having at least about 70% homology thereto.

Exemplary Embodiment 153: The method of any one of embodiments 1-14, 39-59, 62-79, and 152, wherein the ABL1 fusion polypeptide encoded by the ABL1 fusion nucleic acid molecule comprises a fusion between an ABL1 polypeptide, or a portion thereof, fused to a BCR polypeptide, or a portion thereof, wherein the cancer is liver cholangiocarcinoma, and wherein the ABL1 fusion polypeptide:
(a) comprises, in the N- to C-terminus direction, a BCR polypeptide, or a portion thereof, fused to an ABL1 polypeptide, or a portion thereof;
(b) comprises a fusion between an amino acid sequence encoded by exon 14, or a portion thereof, of a BCR gene fused to an amino acid sequence encoded by exon 2, or a portion thereof, of an ABL1 gene;
(c) results from a breakpoint within intron 14 of a BCR gene and/or a breakpoint within intron 1 of an ABL1 gene;
(d) results from a 5′ breakpoint within chromosomal coordinates chr22: 23633837-23634208 and/or a 3′ breakpoint within chromosomal coordinates chr9: 133665426-133665727;
(e) comprises, in the N- to C-terminus direction, an amino acid sequence encoded by exons 1-13 and exon 14, or a portion thereof, of BCR, fused to an amino acid sequence encoded by exon 2, or a portion thereof, and exons 3-11 of ABL1;
(f) is encoded by the nucleotide sequence of SEQ ID NO: 16, or a nucleotide sequence having at least about 70% homology thereto; and/or
(g) comprises the amino acid sequence of SEQ ID NO: 37, or an amino acid sequence having at least about 70% homology thereto.

Exemplary Embodiment 154: The method of any one of embodiments 1-79, wherein the ABL1 fusion nucleic acid molecule comprises a fusion between an ABL1 gene, or a portion thereof, fused to a BCR gene, or a portion thereof, wherein the cancer is pancreas ductal adenocarcinoma, and wherein the ABL1 fusion nucleic acid molecule:
(a) comprises, in 5′ to 3′ direction a BCR gene, or a portion thereof, fused to an ABL1 gene, or a portion thereof;
(b) comprises a fusion between exon 14, or a portion thereof, of a BCR gene fused to exon 2, or a portion thereof, of an ABL1 gene;
(c) comprises or results from a breakpoint within intron 14 of a BCR gene and/or a breakpoint within intron 1 of an ABL1 gene;
(d) comprises or results from a 5′ breakpoint within chromosomal coordinates chr22: 23632858-23633068 and/or a 3′ breakpoint within chromosomal coordinates chr9: 133701595-133701846;
(e) comprises, in 5′ to 3′ direction, exons 1-13 and exon 14, or a portion thereof, of a BCR gene, fused to exon 2, or a portion thereof, and exons 3-11 of an ABL1 gene;
(f) comprises the nucleotide sequence of SEQ ID NO: 17, or a nucleotide sequence having at least about 70% homology thereto; and/or
(g) encodes an ABL1 fusion polypeptide comprising the amino acid sequence of SEQ ID NO: 38, or an amino acid sequence having at least about 70% homology thereto.

Exemplary Embodiment 155: The method of any one of embodiments 1-14, 39-59, 62-79, and 154, wherein the ABL1 fusion polypeptide encoded by the ABL1 fusion nucleic acid molecule comprises a fusion between an ABL1 polypeptide, or a portion thereof, fused to a BCR polypeptide, or a portion thereof, wherein the cancer is pancreas ductal adenocarcinoma, and wherein the ABL1 fusion polypeptide:
(a) comprises, in the N- to C-terminus direction, a BCR polypeptide, or a portion thereof, fused to an ABL1 polypeptide, or a portion thereof;
(b) comprises a fusion between an amino acid sequence encoded by exon 14, or a portion thereof, of a BCR gene fused to an amino acid sequence encoded by exon 2, or a portion thereof, of an ABL1 gene;
(c) results from a breakpoint within intron 14 of a BCR gene and/or a breakpoint within intron 1 of an ABL1 gene;
(d) results from a 5′ breakpoint within chromosomal coordinates chr22: 23632858-23633068 and/or a 3′ breakpoint within chromosomal coordinates chr9: 133701595-133701846;
(e) comprises, in the N- to C-terminus direction, an amino acid sequence encoded by exons 1-13 and exon 14, or a portion thereof, of a BCR gene fused to an amino acid sequence encoded by exon 2, or a portion thereof, and exons 3-11 of an ABL1 gene;
(f) is encoded by the nucleotide sequence of SEQ ID NO: 17, or a nucleotide sequence having at least about 70% homology thereto; and/or
(g) comprises the amino acid sequence of SEQ ID NO: 38, or an amino acid sequence having at least about 70% homology thereto.

Exemplary Embodiment 156: The method of any one of embodiments 1-79, wherein the ABL1 fusion nucleic acid molecule comprises a fusion between an ABL1 gene, or a portion thereof, fused to a BCR gene, or a portion thereof, wherein the cancer is ovary serous carcinoma, and wherein the ABL1 fusion nucleic acid molecule:
(a) comprises, in 5′ to 3′ direction a BCR gene, or a portion thereof, fused to an ABL1 gene, or a portion thereof;
(b) comprises a fusion between exon 14, or a portion thereof, of a BCR gene fused to exon 2, or a portion thereof, of an ABL1 gene;
(c) comprises or results from a breakpoint within intron 14 of a BCR gene and/or a breakpoint within intron 1 of an ABL1 gene;
(d) comprises or results from a 5′ breakpoint within chromosomal coordinates chr22: 23633208-23633695 and/or a 3′ breakpoint within chromosomal coordinates chr9: 133702617-133702992;
(e) comprises, in 5′ to 3′ direction, exons 1-13 and exon 14, or a portion thereof, of a BCR gene, fused to exon 2, or a portion thereof, and exons 3-11 of an ABL1 gene;
(f) comprises the nucleotide sequence of SEQ ID NO: 18, or a nucleotide sequence having at least about 70% homology thereto; and/or
(g) encodes an ABL1 fusion polypeptide comprising the amino acid sequence of SEQ ID NO: 39, or an amino acid sequence having at least about 70% homology thereto.

Exemplary Embodiment 157: The method of any one of embodiments 1-14, 39-59, 62-79, and 156, wherein the ABL1 fusion polypeptide encoded by the ABL1 fusion nucleic acid molecule comprises a fusion between an ABL1 polypeptide, or a portion thereof, fused to a BCR polypeptide, or a portion thereof, wherein the cancer is ovary serous carcinoma, and wherein the ABL1 fusion polypeptide:
(a) comprises, in the N- to C-terminus direction, a BCR polypeptide, or a portion thereof, fused to an ABL1 polypeptide, or a portion thereof;
(b) comprises a fusion between an amino acid sequence encoded by exon 14, or a portion thereof, of a BCR gene fused to an amino acid sequence encoded by exon 2, or a portion thereof, of an ABL1 gene;
(c) results from a breakpoint within intron 14 of a BCR gene and/or a breakpoint within intron 1 of an ABL1 gene;
(d) results from a 5′ breakpoint within chromosomal coordinates chr22: 23633208-23633695 and/or a 3′ breakpoint within chromosomal coordinates chr9: 133702617-133702992;
(e) comprises, in the N- to C-terminus direction, an amino acid sequence encoded by exons 1-13 and exon 14, or a portion thereof, of a BCR gene, fused to an amino acid sequence encoded by exon 2, or a portion thereof, and exons 3-11 of an ABL1 gene;
(f) is encoded by the nucleotide sequence of SEQ ID NO: 18, or a nucleotide sequence having at least about 70% homology thereto; and/or
(g) comprises the amino acid sequence of SEQ ID NO: 39, or an amino acid sequence having at least about 70% homology thereto.

Exemplary Embodiment 158: The method of any one of embodiments 1-79, wherein the ABL1 fusion nucleic acid molecule comprises a fusion between an ABL1 gene, or a portion thereof, fused to a BCR gene, or a portion thereof, wherein the cancer is lung squamous cell carcinoma (SCC), and wherein the ABL1 fusion nucleic acid molecule:
(a) comprises, in 5′ to 3′ direction a BCR gene, or a portion thereof, fused to an ABL1 gene, or a portion thereof;
(b) comprises a fusion between exon 14, or a portion thereof, of a BCR gene fused to exon 2, or a portion thereof, of an ABL1 gene;
(c) comprises or results from a breakpoint within intron 14 of a BCR gene and/or a breakpoint within intron 1 of an ABL1 gene;
(d) comprises or results from:
   a 5′ breakpoint within chromosomal coordinates chr22: 23634171-23634647 and/or a 3′ breakpoint within chromosomal coordinates chr9: 133638886-133639512, or
   a 5′ breakpoint within chromosomal coordinates chr22: 23633910-23634678 and/or a 3′ breakpoint within chromosomal coordinates chr9: 133724778-133725351; and/or
(e) comprises, in 5′ to 3′ direction, exons 1-13 and exon 14, or a portion thereof, of a BCR gene, fused to exon 2, or a portion thereof, and exons 3-11 of an ABL1 gene.

Exemplary Embodiment 159: The method of any one of embodiments 1-14, 39-59, 62-79, and 158, wherein the ABL1 fusion polypeptide encoded by the ABL1 fusion nucleic acid molecule comprises a fusion between an ABL1 polypeptide, or a portion thereof, fused to a BCR polypeptide, or a portion thereof, wherein the cancer is lung squamous cell carcinoma (SCC), and wherein the ABL1 fusion polypeptide:
(a) comprises, in the N- to C-terminus direction, a BCR polypeptide, or a portion thereof, fused to an ABL1 polypeptide, or a portion thereof;
(b) comprises a fusion between an amino acid sequence encoded by exon 14, or a portion thereof, of a BCR gene fused to an amino acid sequence encoded by exon 2, or a portion thereof, of an ABL1 gene;
(c) results from a breakpoint within intron 14 of a BCR gene and/or a breakpoint within intron 1 of an ABL1 gene;
(d) results from:
   a 5′ breakpoint within chromosomal coordinates chr22: 23634171-23634647 and/or a 3′ breakpoint within chromosomal coordinates chr9: 133638886-133639512, or
   a 5′ breakpoint within chromosomal coordinates chr22: 23633910-23634678 and/or a 3′ breakpoint within chromosomal coordinates chr9: 133724778-133725351; and/or
(e) comprises, in the N- to C-terminus direction, an amino acid sequence encoded by exons 1-13 and exon 14, or a portion thereof, of a BCR gene fused to an amino acid sequence encoded by exon 2, or a portion thereof, and exons 3-11 of an ABL1 gene.

Exemplary Embodiment 160: The method of any one of embodiments 1-79, wherein the ABL1 fusion nucleic acid molecule comprises a fusion between an ABL1 gene, or a portion thereof, fused to a NUP214 gene, or a portion thereof, wherein the cancer is bone marrow leukemia lymphocytic chronic (CLL), and wherein the ABL1 fusion nucleic acid molecule:
(a) comprises, in 5′ to 3′ direction, a NUP214 gene, or a portion thereof, fused to an ABL1 gene, or a portion thereof;
(b) comprises a fusion between exon 34, or a portion thereof, of a NUP214 gene fused to exon 3, or a portion thereof, of an ABL1 gene;
(c) comprises or results from a 5′ breakpoint within chromosomal coordinates chr9: 134106039-134106079 and/or a 3′ breakpoint within chromosomal coordinates chr9: 133730222-133730262;
(d) comprises, in 5′ to 3′ direction, exons 1-33 and exon 34, or a portion thereof, of a NUP214 gene, fused to exon 3, or a portion thereof, and exons 4-11 of an ABL1 gene;
(e) comprises the nucleotide sequence of SEQ ID NO: 19, or a nucleotide sequence having at least about 70% homology thereto; and/or
(f) encodes an ABL1 fusion polypeptide comprising the amino acid sequence of SEQ ID NO: 40, or an amino acid sequence having at least about 70% homology thereto.

Exemplary Embodiment 161: The method of any one of embodiments 1-14, 39-59, 62-79, and 160, wherein the ABL1 fusion polypeptide encoded by the ABL1 fusion nucleic acid molecule comprises a fusion between an ABL1 polypeptide, or a portion thereof, fused to a NUP214 polypeptide, or a portion thereof, wherein the cancer is bone marrow leukemia lymphocytic chronic (CLL), and wherein the ABL1 fusion polypeptide:
(a) comprises, in the N- to C-terminus direction, a NUP214 polypeptide, or a portion thereof, fused to an ABL1 polypeptide, or a portion thereof;
(b) comprises a fusion between an amino acid sequence encoded by exon 34, or a portion thereof, of a NUP214 gene fused to an amino acid sequence encoded by exon 3, or a portion thereof, of an ABL1 gene;
(c) results from a 5′ breakpoint within chromosomal coordinates chr9: 134106039-134106079 and/or a 3′ breakpoint within chromosomal coordinates chr9: 133730222-133730262;
(d) comprises, in the N- to C-terminus direction, an amino acid sequence encoded by exons 1-33 and exon 34, or a portion thereof, of a NUP214 gene fused to an amino acid sequence encoded by exon 3, or a portion thereof, and exons 4-11 of an ABL1 gene;
(e) is encoded by the nucleotide sequence of SEQ ID NO: 19, or a nucleotide sequence having at least about 70% homology thereto; and/or
(f) comprises the amino acid sequence of SEQ ID NO: 40, or an amino acid sequence having at least about 70% homology thereto.

Exemplary Embodiment 162: The method of any one of embodiments 1-79, wherein the ABL1 fusion nucleic acid molecule comprises a fusion between an ABL1 gene, or a portion thereof, fused to a NUP214 gene, or a portion thereof, wherein the cancer is soft tissue liposarcoma, and wherein the ABL1 fusion nucleic acid molecule:
(a) comprises, in 5′ to 3′ direction, an ABL1 gene, or a portion thereof, fused to a NUP214 gene, or a portion thereof;
(b) comprises a fusion between exon 1, or a portion thereof, of an ABL1 gene fused to exon 31, or a portion thereof, of a NUP214 gene;
(c) comprises or results from a 5′ breakpoint within chromosomal coordinates chr9: 134090583-134090623 and/or a 3′ breakpoint within chromosomal coordinates chr9: 133710830-133710870; and/or
(d) comprises, in 5′ to 3′ direction, exon 1, or a portion thereof, of an ABL1 gene, fused to exon 31, or a portion thereof, and exons 32-36 of NUP214.

Exemplary Embodiment 163: The method of any one of embodiments 1-14, 39-59, 62-79, and 162, wherein the ABL1 fusion polypeptide encoded by the ABL1 fusion nucleic acid molecule comprises a fusion between an ABL1 polypeptide, or a portion thereof, fused to a NUP214 polypeptide, or a portion thereof, wherein the cancer is soft tissue liposarcoma, and wherein the ABL1 fusion polypeptide:
(a) comprises, in the N- to C-terminus direction, an ABL1 polypeptide, or a portion thereof, fused to a NUP214 polypeptide, or a portion thereof;
(b) comprises a fusion between an amino acid sequence encoded by exon 1, or a portion thereof, of an ABL1 gene fused to an amino acid sequence encoded by exon 31, or a portion thereof, of a NUP214 gene;
(c) results from a 5′ breakpoint within chromosomal coordinates chr9: 134090583-134090623 and/or a 3′ breakpoint within chromosomal coordinates chr9: 133710830-133710870; and/or
(d) comprises, in the N- to C-terminus direction, an amino acid sequence encoded by exon 1, or a portion thereof, of an ABL1 gene, fused to an amino acid sequence encoded by exon 31, or a portion thereof, and exons 32-36 of a NUP214 gene.

Exemplary Embodiment 164: The method of any one of embodiments 1-79, wherein the ABL1 fusion nucleic acid molecule comprises a fusion between an ABL1 gene, or a portion thereof, fused to a AIF1L gene, or a portion thereof, wherein the cancer is soft tissue sarcoma, and wherein the ABL1 fusion nucleic acid molecule:
(a) comprises, in 5′ to 3′ direction, an ABL1 gene, or a portion thereof, fused to an AIF1L gene, or a portion thereof;
(b) comprises a fusion between exon 10, or a portion thereof, of an ABL1 gene fused to exon 4, or a portion thereof, of an AIF1L gene;
(c) comprises or results from a 5′ breakpoint within chromosomal coordinates chr9: 133755952-133755992 and/or a 3′ breakpoint within chromosomal coordinates chr9: 133989965-133990005;
(d) comprises, in 5′ to 3′ direction, exons 1-9 and exon 10, or a portion thereof, of an ABL1 gene, fused to exon 4, or a portion thereof, and exons 5-6 of an AIF1L gene;
(e) comprises the nucleotide sequence of SEQ ID NO: 20, or a nucleotide sequence having at least about 70% homology thereto; and/or
(f) encodes an ABL1 fusion polypeptide comprising the amino acid sequence of SEQ ID NO: 41, or an amino acid sequence having at least about 70% homology thereto;
optionally wherein the soft tissue sarcoma is not otherwise specified (NOS).

Exemplary Embodiment 165: The method of any one of embodiments 1-14, 39-59, 62-79, and 164, wherein the ABL1 fusion polypeptide encoded by the ABL1 fusion nucleic acid molecule comprises a fusion between an ABL1 polypeptide, or a portion thereof, fused to an AIF1L polypeptide, or a portion thereof, wherein the cancer is soft tissue sarcoma, and wherein the ABL1 fusion polypeptide:
(a) comprises, in the N- to C-terminus direction, an ABL1 polypeptide, or a portion thereof, fused to an AIF1L polypeptide, or a portion thereof;
(b) comprises a fusion between an amino acid sequence encoded by exon 10, or a portion thereof, of an ABL1 gene fused to an amino acid sequence encoded by exon 4, or a portion thereof, of an AIF1L gene;
(c) results from a 5′ breakpoint within chromosomal coordinates chr9: 133755952-133755992 and/or a 3′ breakpoint within chromosomal coordinates chr9: 133989965-133990005;
(d) comprises, in the N- to C-terminus direction, an amino acid sequence encoded by exons 1-9 and exon 10, or a portion thereof, of an ABL1 gene fused to an amino acid sequence encoded by exon 4, or a portion thereof, and exons 5-6 of an AIF1L gene;
(e) is encoded by the nucleotide sequence of SEQ ID NO: 20, or a nucleotide sequence having at least about 70% homology thereto; and/or
(f) comprises the amino acid sequence of SEQ ID NO: 41, or an amino acid sequence having at least about 70% homology thereto;
optionally wherein the soft tissue sarcoma is not otherwise specified (NOS).

Exemplary Embodiment 166: The method of any one of embodiments 1-79, wherein the ABL1 fusion nucleic acid molecule comprises a fusion between an ABL1 gene, or a portion thereof, fused to a AIF1L gene, or a portion thereof, wherein the cancer is ovary serous carcinoma, and wherein the ABL1 fusion nucleic acid molecule:
(a) comprises, in 5′ to 3′ direction, an AIF1L gene, or a portion thereof, fused to an ABL1 gene, or a portion thereof;
(b) comprises a fusion between exon 2, or a portion thereof, of an AIF1L gene fused to exon 2, or a portion thereof, of an ABL1 gene;
(c) comprises or results from a breakpoint within intron 2 of an AIF1L gene and/or a breakpoint within intron 1 of an ABL1 gene;
(d) comprises or results from a 5′ breakpoint within chromosomal coordinates chr9: 133978032-133978137 and/or a 3′ breakpoint within chromosomal coordinates chr9: 133729409-133729618; and/or
(e) comprises, in 5′ to 3′ direction, exon 1 and exon 2, or a portion thereof, of an AIF1L gene, fused to exon 2, or a portion thereof, and exons 3-11 of an ABL1 gene.

Exemplary Embodiment 167: The method of any one of embodiments 1-14, 39-59, 62-79, and 166, wherein the ABL1 fusion polypeptide encoded by the ABL1 fusion nucleic acid molecule comprises a fusion between an ABL1 polypeptide, or a portion thereof, fused to a AIF1L polypeptide, or a portion thereof, wherein the cancer is ovary serous carcinoma, and wherein the ABL1 fusion polypeptide:
(a) comprises, in the N- to C-terminus direction, an AIF1L polypeptide, or a portion thereof, fused to an ABL1 polypeptide, or a portion thereof;
(b) comprises a fusion between an amino acid sequence encoded by exon 2, or a portion thereof, of an AIF1L gene fused to an amino acid sequence encoded by exon 2, or a portion thereof, of an ABL1 gene;
(c) results from a breakpoint within intron 2 of an AIF1L gene and/or a breakpoint within intron 1 of an ABL1 gene;
(d) results from a 5′ breakpoint within chromosomal coordinates chr9: 133978032-133978137 and/or a 3′ breakpoint within chromosomal coordinates chr9: 133729409-133729618; and/or
(e) comprises, in the N- to C-terminus direction, an amino acid sequence encoded by exon 1 and exon 2, or a portion thereof, of an AIF1L gene fused to an amino acid sequence encoded by exon 2, or a portion thereof, and exons 3-11 of an ABL1 gene.

Exemplary Embodiment 168: The method of any one of embodiments 1-79, wherein the ABL1 fusion nucleic acid molecule comprises a fusion between an ABL1 gene, or a portion thereof, fused to a RCSD1 gene, or a portion thereof, wherein the cancer is bone marrow leukemia lymphocytic chronic (CLL), and wherein the ABL1 fusion nucleic acid molecule:
(a) comprises, in 5′ to 3′ direction an RCSD1 gene, or a portion thereof, fused to an ABL1 gene, or a portion thereof;
(b) comprises a fusion between exon 4, or a portion thereof, of an RCSD1 gene fused to exon 4, or a portion thereof, of an ABL1 gene;
(c) comprises or results from a 5′ breakpoint within chromosomal coordinates chr1: 167654668-167654708 and/or a 3′ breakpoint within chromosomal coordinates chr9: 133738173-133738213;
(d) comprises, in 5′ to 3′ direction, exons 1-3 and exon 4, or a portion thereof, of a RCSD1 gene, fused to exon 4, or a portion thereof, and exons 5-11 of an ABL1 gene;
(e) comprises the nucleotide sequence of SEQ ID NO: 21, or a nucleotide sequence having at least about 70% homology thereto; and/or
(f) encodes an ABL1 fusion polypeptide comprising the amino acid sequence of SEQ ID NO: 42, or an amino acid sequence having at least about 70% homology thereto.

Exemplary Embodiment 169: The method of any one of embodiments 1-14, 39-59, 62-79, and 168, wherein the ABL1 fusion polypeptide encoded by the ABL1 fusion nucleic acid molecule comprises a fusion between an ABL1 polypeptide, or a portion thereof, fused to a RCSD1 polypeptide, or a portion thereof, wherein the cancer is bone marrow leukemia lymphocytic chronic (CLL), and wherein the ABL1 fusion polypeptide:
(a) comprises, in the N- to C-terminus direction, an RCSD1 polypeptide, or a portion thereof, fused to an ABL1 polypeptide, or a portion thereof;
(b) comprises a fusion between an amino acid sequence encoded by exon 4, or a portion thereof, of an RCSD1 gene fused to an amino acid sequence encoded by exon 4, or a portion thereof, of an ABL1 gene;
(c) results from a 5′ breakpoint within chromosomal coordinates chr1: 167654668-167654708 and/or a 3′ breakpoint within chromosomal coordinates chr9: 133738173-133738213;
(d) comprises, in the N- to C-terminus direction, an amino acid sequence encoded by exons 1-3 and exon 4, or a portion thereof, of an RCSD1 gene, fused to an amino acid sequence encoded by exon 4, or a portion thereof, and exons 5-11 of an ABL1 gene;
(e) is encoded by the nucleotide sequence of SEQ ID NO: 21, or a nucleotide sequence having at least about 70% homology thereto; and/or
(f) comprises the amino acid sequence of SEQ ID NO: 42, or an amino acid sequence having at least about 70% homology thereto.

Exemplary Embodiment 170: The method of any one of embodiments 1-79, wherein the ABL1 fusion nucleic acid molecule comprises a fusion between an ABL1 gene, or a portion thereof, fused to an EXOSC2 gene, or a portion thereof, wherein the cancer is peritoneum adenocarcinoma, and wherein the ABL1 fusion nucleic acid molecule:
(a) comprises, in 5′ to 3′ direction, an EXOSC2 gene, or a portion thereof, fused to an ABL1 gene, or a portion thereof;
(b) comprises a fusion between exon 9, or a portion thereof, of an EXOSC2 gene fused to exon 1, or a portion thereof, of an ABL1 gene;
(c) comprises or results from a breakpoint within exon 9 of an EXOSC2 gene and/or a breakpoint within exon 1 of an ABL1 gene;
(d) comprises or results from a 5′ breakpoint within chromosomal coordinates chr9: 133579154-133579531 and/or a 3′ breakpoint within chromosomal coordinates chr9: 133589786-133589985; and/or
(e) comprises, in 5′ to 3′ direction, exons 1-8 and exon 9, or a portion thereof, of an EXOSC2 gene, fused to exon 1, or a portion thereof, and exons 2-11 of an ABL1 gene.

Exemplary Embodiment 171: The method of any one of embodiments 1-14, 39-59, 62-79, and 170, wherein the ABL1 fusion polypeptide encoded by the ABL1 fusion nucleic acid molecule comprises a fusion between an ABL1 polypeptide, or a portion thereof, fused to an EXOSC2 polypeptide, or a portion thereof, wherein the cancer is peritoneum adenocarcinoma, and wherein the ABL1 fusion polypeptide:
(a) comprises, in the N- to C-terminus direction, an EXOSC2 polypeptide, or a portion thereof, fused to an ABL1 polypeptide, or a portion thereof;
(b) comprises a fusion between an amino acid sequence encoded by exon 9, or a portion thereof, of an EXOSC2 gene fused to an amino acid sequence encoded by exon 1, or a portion thereof, of an ABL1 gene;
(c) results from a breakpoint within exon 9 of an EXOSC2 gene and a breakpoint within exon 1 of an ABL1 gene;
(d) results from a 5′ breakpoint within chromosomal coordinates chr9: 133579154-133579531 and/or a 3′ breakpoint within chromosomal coordinates chr9: 133589786-133589985; and/or
(e) comprises, in the N- to C-terminus direction, an amino acid sequence encoded by exons 1-8 and exon 9, or a portion thereof, of an EXOSC2 gene fused to an amino acid sequence encoded by exon 1, or a portion thereof, and exons 2-11 of an ABL1 gene.

Exemplary Embodiment 172: A kit comprising one or more probes, baits, or oligonucleotides for detecting an ABL1 fusion nucleic acid molecule comprising a fusion between an ABL1 gene, or a portion thereof, fused to:
(a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(b) a second gene, or a portion of a second gene, wherein the ABL1 fusion nucleic acid molecule comprises or results from an ABL1 breakpoint within any of exons 1-4 and 10, or introns 4-5, of ABL1;
(c) a BCR gene, or a portion thereof, wherein the kit is for detecting the ABL1 fusion nucleic acid molecule in a sample from an individual having a cancer, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(d) a NUP214 gene, or a portion thereof, wherein the kit is for detecting the ABL1 fusion nucleic acid molecule in a sample from an individual having a cancer, wherein the cancer is bone marrow leukemia lymphocytic chronic (CLL) or soft tissue liposarcoma;
(e) an AIF1L gene, or a portion thereof, wherein the kit is for detecting the ABL1 fusion nucleic acid molecule in a sample from an individual having a cancer, wherein the cancer is soft tissue sarcoma, or ovary serous carcinoma;
(f) an RCSD1 gene, or a portion thereof, wherein the kit is for detecting the ABL1 fusion nucleic acid molecule in a sample from an individual having a cancer, wherein the cancer is bone marrow leukemia lymphocytic chronic (CLL); or
(g) an EXOSC2 gene, or a portion thereof, wherein the kit is for detecting the ABL1 fusion nucleic acid molecule in a sample from an individual having a cancer, wherein the cancer is peritoneum adenocarcinoma.

Exemplary Embodiment 173: A nucleic acid encoding an ABL1 fusion nucleic acid molecule, or a fragment thereof, comprising a fusion between an ABL1 gene, or a portion thereof, fused to:
(a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; or
(b) a second gene, or a portion of a second gene, wherein the ABL1 fusion nucleic acid molecule comprises or results from an ABL1 breakpoint within any of exons 1-4 and 10, or introns 4-5, of ABL1.

Exemplary Embodiment 174: A vector comprising the nucleic acid of embodiment 173.

Exemplary Embodiment 175: A host cell comprising the vector of embodiment 174.

Exemplary Embodiment 176: An antibody or antibody fragment that specifically binds to an ABL1 fusion polypeptide, or to a portion thereof, wherein the ABL1 fusion polypeptide is encoded by an ABL1 fusion nucleic acid molecule comprising a fusion between an ABL1 gene, or a portion thereof, fused to:
(a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; or
(b) a second gene, or a portion of a second gene, wherein the ABL1 fusion nucleic acid molecule comprises or results from an ABL1 breakpoint within any of exons 1-4 and 10, or introns 4-5, of ABL1.

Exemplary Embodiment 177: A kit comprising the antibody or antibody fragment of embodiment 176.

Exemplary Embodiment 178: In vitro use of one or more probes, baits, or oligonucleotides for detecting an ABL1 fusion nucleic acid molecule comprising a fusion between an ABL1 gene, or a portion thereof, fused to:
(a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(b) a second gene, or a portion of a second gene, wherein the ABL1 fusion nucleic acid molecule comprises or results from an ABL1 breakpoint within any of exons 1-4 and 10, or introns 4-5, of ABL1;
(c) a BCR gene, or a portion thereof, wherein the one or more probes, baits, or oligonucleotides are for use in detecting the ABL1 fusion nucleic acid molecule in a sample from an individual having a cancer, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(d) a NUP214 gene, or a portion thereof, wherein the one or more probes, baits, or oligonucleotides are for use in detecting the ABL1 fusion nucleic acid molecule in a sample from an individual having a cancer, wherein the cancer is bone marrow leukemia lymphocytic chronic (CLL) or soft tissue liposarcoma;
(e) an AIF1L gene, or a portion thereof, wherein the one or more probes, baits, or oligonucleotides are for use in detecting the ABL1 fusion nucleic acid molecule in a sample from an individual having a cancer, wherein the cancer is soft tissue sarcoma, or ovary serous carcinoma;
(f) an RCSD1 gene, or a portion thereof, wherein the one or more probes, baits, or oligonucleotides are for use in detecting the ABL1 fusion nucleic acid molecule in a sample from an individual having a cancer, wherein the cancer is bone marrow leukemia lymphocytic chronic (CLL); or
(g) an EXOSC2 gene, or a portion thereof, wherein the one or more probes, baits, or oligonucleotides are for use in detecting the ABL1 fusion nucleic acid molecule in a sample from an individual having a cancer, wherein the cancer is peritoneum adenocarcinoma.

Exemplary Embodiment 179: An ABL1-targeted therapy for use in a method of treating or delaying progression of cancer, wherein the method comprises administering the ABL1-targeted therapy to an individual having a cancer, wherein:
(1) an ABL1 fusion nucleic acid molecule comprising a fusion between an ABL1 gene, or a portion thereof, fused to:
(a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(b) a second gene, or a portion of a second gene, wherein the ABL1 fusion nucleic acid molecule comprises or results from an ABL1 breakpoint within any of exons 1-4 and 10, or introns 4-5, of ABL1;
(c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(d) a NUP214 gene, or a portion thereof, wherein the cancer is bone marrow leukemia lymphocytic chronic (CLL) or soft tissue liposarcoma;
(e) an AIF1L gene, or a portion thereof, wherein the cancer is soft tissue sarcoma, or ovary serous carcinoma;
(f) an RCSD1 gene, or a portion thereof, wherein the cancer is bone marrow leukemia lymphocytic chronic (CLL); or
(g) an EXOSC2 gene, or a portion thereof, wherein the cancer is peritoneum adenocarcinoma; or
(2) an ABL1 fusion polypeptide encoded by the ABL1 fusion nucleic acid molecule, is detected in a sample obtained from the individual.

Exemplary Embodiment 180: An ABL1-targeted therapy for use in the manufacture of a medicament for treating or delaying progression of cancer, wherein the medicament is to be administered to an individual having a cancer, wherein:
(1) an ABL1 fusion nucleic acid molecule comprising a fusion between an ABL1 gene, or a portion thereof, fused to:
(a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(b) a second gene, or a portion of a second gene, wherein the ABL1 fusion nucleic acid molecule comprises or results from an ABL1 breakpoint within any of exons 1-4 and 10, or introns 4-5, of ABL1;
(c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(d) a NUP214 gene, or a portion thereof, wherein the cancer is bone marrow leukemia lymphocytic chronic (CLL) or soft tissue liposarcoma;
(e) an AIF1L gene, or a portion thereof, wherein the cancer is soft tissue sarcoma, or ovary serous carcinoma;
(f) an RCSD1 gene, or a portion thereof, wherein the cancer is bone marrow leukemia lymphocytic chronic (CLL); or
(g) an EXOSC2 gene, or a portion thereof, wherein the cancer is peritoneum adenocarcinoma; or
(2) an ABL1 fusion polypeptide encoded by the ABL1 fusion nucleic acid molecule, is detected in a sample obtained from the individual.

Exemplary Embodiment 181: A system, comprising:
a memory configured to store one or more program instructions, and one or more processors configured to execute the one or more program instructions, the one or more program instructions when executed by the one or more processors are configured to:
(a) obtain a plurality of sequence reads of one or more nucleic acid molecules, wherein the one or more nucleic acid molecules are derived from a sample obtained from an individual having a cancer;
(b) analyze the plurality of sequence reads for the presence of an ABL1 fusion nucleic acid molecule comprising a fusion between an ABL1 gene, or a portion thereof, fused to:
(i) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(ii) a second gene, or a portion of a second gene, wherein the ABL1 fusion nucleic acid molecule comprises or results from an ABL1 breakpoint within any of exons 1-4 and 10, or introns 4-5, of ABL1;
(iii) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(iv) a NUP214 gene, or a portion thereof, wherein the cancer is bone marrow leukemia lymphocytic chronic (CLL) or soft tissue liposarcoma;
(v) an AIF1L gene, or a portion thereof, wherein the cancer is soft tissue sarcoma, or ovary serous carcinoma;
(vi) an RCSD1 gene, or a portion thereof, wherein the cancer is bone marrow leukemia lymphocytic chronic (CLL); or
(vii) an EXOSC2 gene, or a portion thereof, wherein the cancer is peritoneum adenocarcinoma; and
(c) detect, based on the analyzing, the ABL1 fusion nucleic acid molecule in the sample.

Exemplary Embodiment 182: A non-transitory computer readable storage medium comprising one or more programs executable by one or more computer processors for performing a method, the method comprising:
(a) obtaining, using the one or more processors, a plurality of sequence reads of one or more nucleic acid molecules, wherein the one or more nucleic acid molecules are derived from a sample obtained from an individual having a cancer;
(b) analyzing, using the one or more processors, the plurality of sequence reads for the presence of an ABL1 fusion nucleic acid molecule comprising a fusion between an ABL1 gene, or a portion thereof, fused to:
(i) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(ii) a second gene, or a portion of a second gene, wherein the ABL1 fusion nucleic acid molecule comprises or results from an ABL1 breakpoint within any of exons 1-4 and 10, or introns 4-5, of ABL1;
(iii) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(iv) a NUP214 gene, or a portion thereof, wherein the cancer is bone marrow leukemia lymphocytic chronic (CLL) or soft tissue liposarcoma;
(v) an AIF1L gene, or a portion thereof, wherein the cancer is soft tissue sarcoma, or ovary serous carcinoma;
(vi) an RCSD1 gene, or a portion thereof, wherein the cancer is bone marrow leukemia lymphocytic chronic (CLL); or
(vii) an EXOSC2 gene, or a portion thereof, wherein the cancer is peritoneum adenocarcinoma; and
(c) detecting, using the one or more processors and based on the analyzing, the ABL1 fusion nucleic acid molecule in the sample.

Exemplary Embodiment 183: The system of embodiment 181, or the non-transitory computer readable storage medium of embodiment 182, wherein the plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next generation sequencing (NGS).

Exemplary Embodiment 184: The system of embodiment 181 or embodiment 183, wherein the one or more program instructions when executed by the one or more processors are further configured to generate, based at least in part on the detecting, a genomic profile for the sample.

Exemplary Embodiment 185: The non-transitory computer readable storage medium of embodiment 182 or embodiment 183, wherein the method further comprises generating, based at least in part on the detecting, a genomic profile for the sample.

Exemplary Embodiment 186: The system of embodiment 184, or the non-transitory computer readable storage medium of embodiment 185, wherein the individual is administered a treatment based at least in part on the genomic profile; optionally wherein the treatment comprises an ABL1-targeted therapy.

Exemplary Embodiment 187: The system of any one of embodiments 184 and 186, or the non-transitory computer readable storage medium of 185 or embodiment 186, wherein the genomic profile further comprises results from a comprehensive genomic profiling (CGP) test, a gene expression profiling test, a cancer hotspot panel test, a DNA methylation test, a DNA fragmentation test, an RNA fragmentation test, or any combination thereof.

Exemplary Embodiment 188: The system of any one of embodiments 184 and 186-187, or the non-transitory computer readable storage medium of any one of embodiments 185-187, wherein the genomic profile further comprises results from a nucleic acid sequencing-based test.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes. To the extent that any reference incorporated by reference conflicts with the instant disclosure, the instant disclosure shall control.

### EXAMPLES

The invention will be more fully understood by reference to the following examples. They should not, however, be construed as limiting the scope of the invention. It is understood that the examples and embodiments described herein are for illustrative purposes only, and that various modifications or changes in light thereof will be suggested to persons skilled in the art and are to be included within the spirit and purview of this application and scope of the appended claims.

### Example 1: Identification of Novel ABL1 Gene Fusions in Cancer

This Example describes identification of gene fusions involving the Abelson protein tyrosine kinase (ABL1) gene.

ABL1 gene fusions were identified in a database of comprehensive genomic profiling (CGP) results (see, e.g., Frampton, G. M., et al. (2013) Nat. Biotechnol. 31 (11): 1023-31) of samples from patients with various cancer types. The CGP database was queried using Structured Query Language (SQL), which was limited to fusions, and excluded out-of-strand rearrangements as well as previously described fusions.

As shown in Table 1, ABL1 gene fusions involving numerous gene fusion partners were identified.

TABLE 1
ABL1 gene fusions.

| | Number of |
|---|---|
| | Cases |
| Fusion | Identified |
| ABL1 - APOOL | 1 |
| ABL1 - BMP2K | 1 |
| ABL1 - DNAH2 | 1 |
| ABL1 - RHEX/C1orf186 | 1 |
| ABL1 - EHMT1 | 1 |
| ABL1 - MED27 | 1 |
| ABL1 - RALGPS1 | 1 |
| ABL1 - RFFL | 1 |
| ABL1 - SLC27A4 | 1 |
| ABL1 - TSC1 | 1 |
| ABL1 - TSPAN18 | 1 |
| ABL1 - ZNF804B | 1 |
| ABL1 - BCR | 18 |
| ABL1 - NUP214 | 6 |
| ABL1 - AIF1L | 2 |
| ABL1 - RCSD1 | 1 |
| ABL1 - EXOSC2 | 1 |
| ABL1 - SNX2 | 2 |

As shown in Table 2, detailed characterization of the identified ABL1 gene fusions revealed that ABL1 gene fusions were present in diverse cancer types, and had various fusion partner genes and breakpoints.

TABLE 2
Detailed Characterization of ABL1 fusions.

| Fusion | 5′ breakpoint/ | | Fusion RNA |
|---|---|---|---|
| Exon and breakpoint information | 3′ breakpoint | | sequence |
| Tumor/cancer type | 5′ Exons/ | 5′ Refseq ID/ | Fusion protein |
| Additional information about fusion | 3′ Exons | 3′ Refseq ID | sequence |
| BMP2K-ABL1 | chr4: 79800022- | NM_198892/ | |
| 5′-BMP2K(ex1-15 NM_198892)-ABL1(ex3-11 | 79800062/ | NM_005157 | |
| NM_005157) | chr9: 133730205- | | |
| Bone marrow leukemia T cell acute (T-ALL) | 133730245 | | |
| Fusion is canonical orientation, and includes | BMP2K ex1- | | |
| ABL1 kinase domain. | 15/ ABL1 ex3- | | |
| Non-reciprocal fusion. | 11 | | |
| ABL1 breakpoint in exon 3. | | | |
| EHMT1-ABL1 | chr9: 140630475- | NM_024757/ | SEQ ID NO: 2 |
| 5′-EHMT1(ex1-4 NM_024757)-ABL1(ex2-11 | 140630606/ | NM_005157 | SEQ ID NO: 23 |
| NM_005157) | chr9: 133729408- | | |
| Breakpoints: EHMT1 intron 4, ABL1 intron 1. | 133729648 | | |
| Lung adenocarcinoma | EHMT1 ex1-4/ | | |
| Fusion is in frame, in canonical orientation, and | ABL1 ex2-11 | | |
| includes ABL1 kinase domain. | | | |
| Non-reciprocal fusion. | | | |
| ABL1 breakpoint in intron. | | | |
| MED27-ABL1 | chr9: 134774423- | NM_004269/ | SEQ ID NO: 3 |
| 5′-MED27(ex1-4 NM_004269)-ABL1(ex5-11 | 134776006/ | NM_005157 | SEQ ID NO: 24 |
| NM_005157) | chr9: 133747364- | | |
| Breakpoints: MED27 intron 4, ABL1 intron 4. | 133747762 | | |
| Ovary epithelial carcinoma (NOS) | MED27 ex1-4/ | | |
| Fusion is in frame, in canonical orientation, and | ABL1 ex5-11 | | |
| includes ABL1 kinase domain. | | | |
| Non-reciprocal fusion. | | | |
| ABL1 breakpoint in intron. | | | |
| RFFL-ABL1 | chr17: 33343379- | NM_001017368/ | SEQ ID NO: 4 |
| 5′-RFFL(ex1-5 NM_001017368)-ABL1(ex2-11 | 33343419/ | NM_005157 | SEQ ID NO: 25 |
| NM_005157); | chr9: 133729485- | | |
| Soft tissue malignant peripheral nerve sheath | 133729525 | | |
| tumor (MPNST) | RFFL ex1-5/ | | |
| Fusion is in frame, in canonical orientation, and | ABL1 ex2-11 | | |
| includes ABL1 kinase domain. | | | |
| Non-reciprocal fusion. | | | |
| ABL1 breakpoint in intron. | | | |
| SLC27A4-ABL1 | chr9: 131119603- | NM_005094/ | SEQ ID NO: 5 |
| 5′-SLC27A4(ex1-12 NM_005094)-ABL1(ex6- | 131119801/ | NM_005157 | SEQ ID NO: 26 |
| 11 NM_005157) | chr9: 133748211- | | |
| Breakpoints: SLC27A4 intron 12, ABL1 intron 5. | 133748501 | | |
| Testis germ cell tumor (mixed) | SLC27A4 ex1- | | |
| Fusion is in frame, in canonical orientation, and | 12/ ABL1 ex6- | | |
| includes ABL1 kinase domain. | 11 | | |
| Non-reciprocal fusion. | | | |
| ABL1 breakpoint in intron. | | | |
| BCR-ABL1 | chr22: 23632033- | NM_004327/ | SEQ ID NO: 6 |
| 5′-BCR(ex1-13 NM_004327)-ABL1(ex2-11 | 23632321/ | NM_005157 | SEQ ID NO: 27 |
| NM_005157) | chr9: 133591666- | | |
| Breakpoints: BCR intron 13, ABL1 intron 1. | 133591902 | | |
| Lung adenocarcinoma | BCR ex1-13/ | | |
| Fusion is in frame, in canonical orientation, and | ABL1 ex2-11 | | |
| includes ABL1 kinase domain. | | | |
| Reciprocal fusion. | | | |
| ABL1 breakpoint in intron. | | | |
| BCR-ABL1 | chr22: 23634537- | NM_004327/ | SEQ ID NO: 7 |
| 5′-BCR(ex1-14 NM_004327)-ABL1(ex2-11 | 23634672/ | NM_005157 | SEQ ID NO: 28 |
| NM_005157) | chr9: 133672815- | | |
| Breakpoints: BCR intron 14, ABL1 intron 1. | 133673059 | | |
| Lung adenocarcinoma | BCR ex1-14/ | | |
| Fusion is in frame, in canonical orientation, and | ABL1 ex2-11 | | |
| includes ABL1 kinase domain. | | | |
| Reciprocal fusion. | | | |
| ABL1 breakpoint in intron. | | | |
| BCR-ABL1 | chr22: 23634041- | NM_004327/ | SEQ ID NO: 8 |
| 5′-BCR(ex1-14 NM_004327)-ABL1(ex2-11 | 23634323/ | NM_005157 | SEQ ID NO: 29 |
| NM_005157) | chr9: 133708182- | | |
| Breakpoints: BCR intron 14, ABL1 intron 1. | 133708567 | | |
| Lung adenocarcinoma | BCR ex1-14/ | | |
| Fusion is in frame, in canonical orientation, and | ABL1 ex2-11 | | |
| includes ABL1 kinase domain. | | | |
| Reciprocal fusion. | | | |
| ABL1 breakpoint in intron. | | | |
| BCR-ABL1 | chr22: 23632683- | NM_004327/ | SEQ ID NO: 9 |
| 5′-BCR(ex1-14 NM_004327)-ABL1(ex2-11 | 23632948/ | NM_005157 | SEQ ID NO: 30 |
| NM_005157) | chr9: 133613427- | | |
| Breakpoints: BCR intron 14, ABL1 intron 1. | 133613626 | | |
| Lung adenocarcinoma | BCR ex1-14/ | | |
| Fusion is in frame, in canonical orientation, and | ABL1 ex2-11 | | |
| includes ABL1 kinase domain. | | | |
| Reciprocal fusion. | | | |
| ABL1 breakpoint in intron. | | | |
| BCR-ABL1 | chr22: 23631693- | NM_004327/ | SEQ ID NO: 10 |
| 5′-BCR(ex1-12 NM_004327)-ABL1(ex2-11 | 23632188/ | NM_005157 | SEQ ID NO: 31 |
| NM_005157) | chr9: 133607683- | | |
| Breakpoints: BCR intron 12, ABL1 intron 1. | 133608245 | | |
| Lung adenocarcinoma | BCR ex1-12/ | | |
| Fusion is in frame, in canonical orientation, and | ABL1 ex2-11 | | |
| includes ABL1 kinase domain. | | | |
| Reciprocal fusion. | | | |
| ABL1 breakpoint in intron. | | | |
| BCR-ABL1 | chr22: 23634011- | NM_004327/ | SEQ ID NO: 11 |
| 5′-BCR(ex1-14 NM_004327)-ABL1(ex2-11 | 23634460/ | NM_005157 | SEQ ID NO: 32 |
| NM_005157) | chr9: 133645693- | | |
| Breakpoints: BCR intron 14, ABL1 intron 1. | 133646017 | | |
| Head and neck squamous cell carcinoma | BCR ex1-14/ | | |
| (HNSCC) | ABL1 ex2-11 | | |
| Fusion is in frame, in canonical orientation, and | | | |
| includes ABL1 kinase domain. | | | |
| Non-reciprocal fusion. | | | |
| ABL1 breakpoint in intron. | | | |
| BCR-ABL1 | chr22: 23634211- | NM_004327/ | SEQ ID NO: 12 |
| 5′-BCR(ex1-14 NM_004327)-ABL1(ex2-11 | 23634632/ | NM_005157 | SEQ ID NO: 33 |
| NM_005157) | chr9: 133619101- | | |
| Breakpoints: BCR intron 14, ABL1 intron 1. | 133619395 | | |
| Head and neck squamous cell carcinoma | BCR ex1-14/ | | |
| (HNSCC) | ABL1 ex2-11 | | |
| Fusion is in frame, in canonical orientation, and | | | |
| includes ABL1 kinase domain. | | | |
| Non-reciprocal fusion. | | | |
| ABL1 breakpoint in intron. | | | |
| BCR-ABL1 | chr22: 23631722- | NM_004327/ | SEQ ID NO: 13 |
| 5′-BCR(ex1-13 NM_004327)-ABL1(ex2-11 | 23631762/ | NM_005157 | SEQ ID NO: 34 |
| NM_005157) | chr9: 133729484- | | |
| Breakpoints: BCR intron 13, ABL1 intron 1. | 133729524 | | |
| Bone sarcoma (NOS) | BCR ex1-13/ | | |
| Fusion is in frame, in canonical orientation, and | ABL1 ex2-11 | | |
| includes ABL1 kinase domain. | | | |
| Reciprocal fusion. | | | |
| ABL1 breakpoint in intron. | | | |
| BCR-ABL1 | chr22: 23633346- | NM_004327/ | SEQ ID NO: 14 |
| 5′-BCR(ex1-14 NM_004327)-ABL1(ex2-11 | 23634163/ | NM_005157 | SEQ ID NO: 35 |
| NM_005157) | chr9: 133609201- | | |
| Breakpoints: ABL1 intron 1, BCR intron 14. | 133609909 | | |
| Esophagus adenocarcinoma | BCR ex1-14/ | | |
| Fusion is in frame, in canonical orientation, and | ABL1 ex2-11 | | |
| includes ABL1 kinase domain. | | | |
| Reciprocal fusion. | | | |
| ABL1 breakpoint in intron. | | | |
| BCR-ABL1 | chr22: 23633432- | NM_004327/ | SEQ ID NO: 15 |
| 5′-BCR(ex1-14 NM_004327)-ABL1(ex2-11 | 23633796/ | NM_005157 | SEQ ID NO: 36 |
| NM_005157) | chr9: 133623903- | | |
| Breakpoints: BCR intron 14, ABL1 intron 1. | 133624461 | | |
| Gastroesophageal junction adenocarcinoma | BCR ex1-14/ | | |
| Fusion is in frame, in canonical orientation, and | ABL1 ex2-11 | | |
| includes ABL1 kinase domain. | | | |
| Reciprocal fusion. | | | |
| ABL1 breakpoint in intron. | | | |
| BCR-ABL1 | chr22: 23633837- | NM_004327/ | SEQ ID NO: 16 |
| 5′-BCR(ex1-14 NM_004327)-ABL1(ex2-11 | 23634208/ | NM_005157 | SEQ ID NO: 37 |
| NM_005157) | chr9: 133665426- | | |
| Breakpoints: BCR intron 14, ABL1 intron 1. | 133665727 | | |
| Liver cholangiocarcinoma | BCR ex1-14/ | | |
| Fusion is in frame, in canonical orientation, and | ABL1 ex2-11 | | |
| includes ABL1 kinase domain. | | | |
| Reciprocal fusion. | | | |
| ABL1 breakpoint in intron. | | | |
| BCR-ABL1 | chr22: 23632858- | NM_004327/ | SEQ ID NO: 17 |
| 5′-BCR(ex1-14 NM_004327)-ABL1(ex2-11 | 23633068/ | NM_005157 | SEQ ID NO: 38 |
| NM_005157) | chr9: 133701595- | | |
| Breakpoints: BCR intron 14, ABL1 intron 1. | 133701846 | | |
| Pancreas ductal adenocarcinoma | BCR ex1-14/ | | |
| Fusion is in frame, in canonical orientation, and | ABL1 ex2-11 | | |
| includes ABL1 kinase domain. | | | |
| Reciprocal fusion. | | | |
| ABL1 breakpoint in intron. | | | |
| BCR-ABL1 | chr22: 23633208- | NM_004327/ | SEQ ID NO: 18 |
| 5′-BCR(ex1-14 NM_004327)-ABL1(ex2-11 | 23633695/ | NM_005157 | SEQ ID NO: 39 |
| NM_005157) | chr9: 133702617- | | |
| Breakpoints: BCR intron 14, ABL1 intron 1. | 133702992 | | |
| Ovary serous carcinoma | BCR ex1-14/ | | |
| Fusion is in frame, in canonical orientation, and | ABL1 ex2-11 | | |
| includes ABL1 kinase domain. | | | |
| Reciprocal fusion. | | | |
| ABL1 breakpoint in intron. | | | |
| NUP214-ABL1 | chr9: 134106039- | NM_005085/ | SEQ ID NO: 19 |
| 5′-NUP214(ex1-34 NM_005085)-ABL1(ex3-11 | 134106079/ | NM_005157 | SEQ ID NO: 40 |
| NM_005157) | chr9: 133730222- | | |
| Bone marrow leukemia lymphocytic chronic | 133730262 | | |
| (CLL) | NUP214 ex1- | | |
| Fusion is in frame, in canonical orientation, and | 34/ ABL1 ex3- | | |
| includes ABL1 kinase domain. | 11 | | |
| Non-reciprocal fusion. | | | |
| ABL1 breakpoint in intron. | | | |
| ABL1-AIF1L | chr9: 133755952- | NM_005157/ | SEQ ID NO: 20 |
| 5′-ABL1(ex1-10 NM_005157)-AIFIL(ex4-6 | 133755992/ | NM_031426 | SEQ ID NO: 41 |
| NM_031426) | chr9: 133989965- | | |
| Soft tissue sarcoma (NOS) | 133990005 | | |
| Fusion is in frame and includes ABL1 kinase | ABL1 ex1-10/ | | |
| domain. | AIFIL ex4-6 | | |
| Non-reciprocal fusion. | | | |
| ABL1 breakpoint in intron. | | | |
| RCSD1-ABL1 | chr1: 167654668- | NM_052862/ | SEQ ID NO: 21 |
| 5′-RCSD1(ex1-4 NM_052862)-ABL1(ex4-11 | 167654708/ | NM_005157 | SEQ ID NO: 42 |
| NM_005157) | chr9: 133738173- | | |
| Bone marrow leukemia lymphocytic chronic | 133738213 | | |
| (CLL) | RCSD1 ex1-4/ | | |
| Fusion is in frame, in the canonical orientation, | ABL1 ex4-11 | | |
| and includes ABL1 kinase domain. | | | |
| Non-reciprocal fusion. | | | |
| ABL1 breakpoint in intron. | | | |
| APOOL-ABL1 | chrX: 84302705- | NM_198450/ | |
| 5′-APOOL(ex1-2 NM_198450)-ABL1(ex2-11 | 84302904/ | NM_005157 | |
| NM_005157) | chr9: 133729584- | | |
| Breakpoints: APOOL intron 2, ABL1 exon 2. | 133729766 | | |
| Lung adenocarcinoma | APOOL ex1-2/ | | |
| Fusion is in the canonical orientation, and | ABL1 ex2-11 | | |
| includes ABL1 kinase domain. | | | |
| Non-reciprocal fusion. | | | |
| ABL1 breakpoint in exon. | | | |
| ABL1-DNAH2 | chr9: 133755953- | NM_005157/ | |
| 5′-ABL1(ex1-10 NM_005157)-DNAH2(ex4-85 | 133755993/ | NM_020877 | |
| NM_020877) | chr17: 7636399- | | |
| Soft tissue sarcoma (NOS) | 7636439 | | |
| Fusion includes ABL1 kinase domain. | ABL1 ex1-10/ | | |
| Non-reciprocal fusion. | DNAH2 ex4- | | |
| | 85 | | |
| RHEX/C1orf186-ABL1 | chr1: 206288099- | NM_001007544/ | |
| 5′-RHEX/C1orf186(ex1-1 UTR | 206288139/ | NM_005157 | |
| NM_001007544)-ABL1(ex2-11 NM_005157) | chr9: 133729488- | | |
| Bone marrow multiple myeloma | 133729528 | | |
| Fusion is in the canonical orientation, and | RHEX/C1orf186 | | |
| includes ABL1 kinase domain. | ex1/ ABL1 | | |
| Non-reciprocal fusion | ex2-11 | | |
| “UTR” indicates a part of the 5′ UTR of RHEX | | | |
| without coding sequence. | | | |
| RALGPS1-ABL1 | chr9: 129694023- | NM_014636/ | |
| 5′-RALGPS1(ex1-1 UTR NM_014636)- | 129694282/ | NM_005157 | |
| ABL1(ex3-11 NM_005157) | chr9: 133730118- | | |
| Breakpoints: RALGPS1 intron 1, ABL1 intron | 133730529 | | |
| 2. | RALGPS1 ex1/ | | |
| Unknown primary melanoma | ABL1 ex3-11 | | |
| Fusion is in the canonical orientation, and | | | |
| includes ABL1 kinase domain. | | | |
| Non-reciprocal fusion | | | |
| “UTR” indicates a part of the 5′ UTR of | | | |
| RALGPS1 without coding sequence. | | | |
| TSC1-ABL1 | chr9: 135,776,998- | NM_000368/ | |
| 5′-TSC1(ex1-19 NM_000368)-ABL1(ex2-11 | 135,777,038/ | NM_005157 | |
| NM_005157) | chr9: 133729491- | | |
| Soft tissue malignant peripheral nerve sheath | 133729531 | | |
| tumor (MPNST) | TSC1 ex1-19/ | | |
| Fusion is in the canonical orientation, and | ABL1 ex2-11 | | |
| includes ABL1 kinase domain. | | | |
| Non-reciprocal fusion | | | |
| TSPAN18-ABL1 | chr11: 44881929- | NM_130783/ | |
| 5′-TSPAN18(ex1-2 UTR NM_130783)- | 44881969/ | NM_005157 | |
| ABL1(ex2-11 NM_005157) | chr9: 133729517- | | |
| Soft tissue osteosarcoma (extraskeletal) | 133729557 | | |
| Fusion is in the canonical orientation, and | TSPAN18 ex1- | | |
| includes ABL1 kinase domain. | 2/ ABL1 ex2- | | |
| Non-reciprocal fusion. | 11 | | |
| “UTR” indicates a part of the 5′ UTR of TSPAN18 | | | |
| without coding sequence. | | | |
| ZNF804B-ABL1 | chr7: 88521265- | NM_181646/ | |
| 5′-ZNF804B(ex1-1 NM_181646)-ABL1(ex2-11 | 88521397/ | NM_005157 | |
| NM_005157) | chr9: 133729414- | | |
| Breakpoints: ZNF804B intron 1, ABL1 intron 1. | 133729519 | | |
| Skin melanoma | ZNF804B ex1/ | | |
| Fusion is in the canonical orientation, and | ABL1 ex2-11 | | |
| includes ABL1 kinase domain. | | | |
| Non-reciprocal fusion | | | |
| BCR-ABL1 | chr22: 23631687- | NM_004327/ | |
| 5′-BCR(ex1-13 NM_004327)-ABL1(ex2-11 | 23632492/ | NM_007313 | |
| NM_007313) | chr9: 133604425- | | |
| Breakpoints BCR intron 13, ABL1 intron 1. | 133604577 | | |
| Head and neck squamous cell carcinoma | BCR ex1-13/ | | |
| (HNSCC) | ABL1 ex2-11 | | |
| Fusion is in the canonical orientation, and | | | |
| includes ABL1 kinase domain. | | | |
| Reciprocal fusion. | | | |
| ABL1 breakpoint in intron. | | | |
| BCR-ABL1 | chr22: 23632288- | NM_004327/ | |
| 5′-BCR(ex1-14 NM_004327)-ABL1(ex2-11 | 23632656/ | NM_007313 | |
| NM_007313) | chr9: 133663291- | | |
| Breakpoints: BCR intron 14, ABL1 intron 1. | 133663682 | | |
| Head and neck squamous cell carcinoma | BCR ex1-14/ | | |
| (HNSCC) | ABL1 ex2-11 | | |
| Fusion is in the canonical orientation, and | | | |
| includes ABL1 kinase domain. | | | |
| Reciprocal fusion. | | | |
| ABL1 breakpoint in intron. | | | |
| BCR-ABL1 | chr22: 23634171- | NM_004327/ | |
| 5′-BCR(ex1-14 NM_004327)-ABL1(ex2-11 | 23634647/ | NM_007313 | |
| NM_007313) | chr9: 133638886- | | |
| Breakpoints: BCR intron 14, ABL1 intron 1. | 133639512 | | |
| Lung squamous cell carcinoma (SCC) | BCR ex1-14/ | | |
| Fusion is in the canonical orientation, and | ABL1 ex2-11 | | |
| includes ABL1 kinase domain. | | | |
| Reciprocal fusion. | | | |
| ABL1 breakpoint in intron. | | | |
| BCR-ABL1 | chr22: 23633910- | NM_004327/ | |
| 5′-BCR(ex1-14 NM_004327)-ABL1(ex2-11 | 23634678/ | NM_005157 | |
| NM_005157) | chr9: 133724778- | | |
| Breakpoints: BCR intron 14, ABL1 intron 1. | 133725351 | | |
| Lung squamous cell carcinoma (SCC) | BCR ex1-14/ | | |
| Fusion is in the canonical orientation, and | ABL1 ex2-11 | | |
| includes ABL1 kinase domain. | | | |
| Reciprocal fusion. | | | |
| ABL1 breakpoint in intron. | | | |
| ABL1-NUP214 | chr9: 134090583- | NM_005157/ | |
| 5′-ABL1(ex1-1 NM_005157)-NUP214(ex31-36 | 134090623/ | NM_005085 | |
| NM_005085) | chr9: 133710830- | | |
| Soft tissue liposarcoma | 133710870 | | |
| Non-reciprocal fusion. | ABL1 ex1/ | | |
| | NUP214 ex31- | | |
| | 36 | | |
| AIF1L-ABL1 | chr9: 133978032- | NM_031426/ | |
| 5′-AIFIL(ex1-2 NM_031426)-ABL1(ex2-11 | 133978137/ | NM_005157 | |
| NM_005157) | chr9: 133729409- | | |
| Breakpoints: AIFIL intron 2, ABL1 intron 1. | 133729618 | | |
| Ovary serous carcinoma | AIFIL ex1-2/ | | |
| Fusion is in the canonical orientation, and | ABL1 ex2-11 | | |
| includes ABL1 kinase domain. | | | |
| Non-reciprocal fusion. | | | |
| EXOSC2-ABL1 | chr9: 133579154- | NM_014285/ | |
| 5′-EXOSC2(ex1-9 UTR NM_014285)- | 133579531/ | NM_007313 | |
| ABL1(ex1-11 NM_007313) | chr9: 133589786- | | |
| Breakpoints: EXOSC2 exon 9, ABL1 exon 1. | 133589985 | | |
| Peritoneum adenocarcinoma | EXOSC2 ex1- | | |
| Fusion is in the canonical orientation, and | 9/ ABL1 ex1- | | |
| includes ABL1 kinase domain. | 11 | | |
| Non-reciprocal fusion. | | | |
| ABL1 breakpoint in exon. | | | |
| “UTR” indicates a part of the 3′ UTR of | | | |
| EXOSC2. | | | |
| NUP214-ABL1 | chr9: 134106080- | NM_005085/ | |
| 5′-NUP214(ex1-34 NM_005085)-ABL1(ex3-11 | 134106120/ | NM_005157 | |
| NM_005157) | chr9: 133730195- | | |
| Breakpoints: NUP214 intron 34, ABL1 exon 3. | 133730235 | | |
| Bone marrow leukemia B cell acute (B-ALL) | NUP214 ex1- | | |
| Fusion is in the canonical orientation, and | 34/ ABL1 ex3- | | |
| includes ABL1 kinase domain. | 11 | | |
| Non-reciprocal fusion. | | | |
| ABL1 breakpoint in exon. | | | |
| NUP214-ABL1 | chr9: 134106030- | NM_005085/ | |
| 5′-NUP214(ex1-34 NM_005085)-ABL1(ex3-11 | 134106070/ | NM_005157 | |
| NM_005157) | chr9: 133730218- | | |
| Bone marrow leukemia B cell acute (B-ALL) | 133730258 | | |
| Fusion is in the canonical orientation, and | NUP214 ex1- | | |
| includes ABL1 kinase domain. | 34/ ABL1 ex3- | | |
| Non-reciprocal fusion. | 11 | | |
| ABL1 breakpoint in exon. | | | |
| NUP214-ABL1 | chr9: 134106046- | NM_005085/ | |
| 5′-NUP214(ex1-34 NM_005085)-ABL1(ex3-11 | 134106086/ | NM_005157 | |
| NM_005157) | chr9: 133730203- | | |
| Bone marrow leukemia B cell acute (B-ALL) | 133730243 | | |
| Also seen in DNA. | NUP214 ex1- | | |
| Fusion is in the canonical orientation, and | 34/ ABL1 ex3- | | |
| includes ABL1 kinase domain. | 11 | | |
| Non-reciprocal fusion. | | | |
| ABL1 breakpoint in exon. | | | |
| NUP214-ABL1 | chr9: 134106028- | NM_005085/ | |
| 5′-NUP214(ex1-34 NM_005085)-ABL1(ex3-11 | 134106068/ | NM_005157 | |
| NM_005157) | chr9: 133730234- | | |
| Bone marrow leukemia lymphocytic acute | 133730274 | | |
| (ALL) | NUP214 ex1- | | |
| Fusion is in the canonical orientation, and | 34/ ABL1 ex3- | | |
| includes ABL1 kinase domain. | 11 | | |
| Non-reciprocal fusion. | | | |
| ABL1 breakpoint in exon. | | | |
| BCR-ABL1 | chr22: 23524272- | NM_004327/ | |
| 5′-BCR(ex1-1 NM_004327)-ABL1(ex3-11 | 23524312/ | NM_005157 | |
| NM_005157) | chr9: 133730218- | | |
| Bone marrow leukemia lymphocytic acute | 133730258 | | |
| (ALL) | BCR ex1/ | | |
| Fusion is in the canonical orientation, and | ABL1 ex3-11 | | |
| includes ABL1 kinase domain. | | | |
| Non-reciprocal fusion. | | | |
| ABL1 breakpoint in exon. | | | |
| SNX2-ABL1 | chr5: 122135451- | NM_003100/ | |
| 5′-SNX2(ex1-3 NM_003100)-ABL1(ex4-11 | 122135491/ | NM_005157 | |
| NM_005157); | chr9: 133738222- | | |
| Bone marrow leukemia B cell acute (B-ALL) | 133738262 | | |
| Fusion is in the canonical orientation, and | SNX2 ex1-3/ | | |
| includes ABL1 kinase domain. | ABL1 ex4-11 | | |
| Reciprocal fusion. | | | |
| ABL1 breakpoint in exon. | | | |
| SNX2-ABL1 | chr5: 122135445- | NM_003100/ | |
| 5′-SNX2(ex1-3 NM_003100)-ABL1(ex4-11 | 122135485/ | NM_005157 | |
| NM_005157). | chr9: 133738214- | | |
| B-lymphoblastic leukemia-lymphoma (B-ALL) | 133738254 | | |
| Fusion is in the canonical orientation, and | SNX2 ex1-3/ | | |
| includes ABL1 kinase domain. | ABL1 ex4-11 | | |
| Reciprocal fusion. | | | |
| ABL1 breakpoint in exon. | | | |

What is claimed is:

1. A method of treating or delaying progression of cancer, comprising: detecting in a sample from an individual having a cancer an Abelson tyrosine-protein kinase 1 (ABL1) fusion nucleic acid molecule, or an ABL1 fusion polypeptide encoded by the ABL1 fusion nucleic acid molecule, wherein the ABL1 fusion nucleic acid molecule comprises a fusion between an ABL1 gene, or a portion thereof, fused to:
(a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(b) a second gene, or a portion of a second gene, wherein the ABL1 fusion nucleic acid molecule comprises or results from an ABL1 breakpoint within any of exons 1-4 and 10, or introns 4-5, of ABL1;
(c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(d) a NUP214 gene, or a portion thereof, wherein the cancer is bone marrow leukemia lymphocytic chronic (CLL) or soft tissue liposarcoma;
(e) an AIF1L gene, or a portion thereof, wherein the cancer is soft tissue sarcoma, or ovary serous carcinoma;
(f) an RCSD1 gene, or a portion thereof, wherein the cancer is bone marrow leukemia lymphocytic chronic (CLL); or
(g) an EXOSC2 gene, or a portion thereof, wherein the cancer is peritoneum adenocarcinoma; and
administering to the individual an effective amount of a treatment that comprises an ABL1-targeted therapy.

2. A method of identifying one or more treatment options for an individual having a cancer, the method comprising:
(1) detecting or acquiring knowledge of an ABL1 fusion nucleic acid molecule, or an ABL1 fusion polypeptide encoded by the ABL1 fusion nucleic acid molecule, in a sample from the individual, wherein the ABL1 fusion nucleic acid molecule comprises a fusion between an ABL1 gene, or a portion thereof, fused to:
(a) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(b) a second gene, or a portion of a second gene, wherein the ABL1 fusion nucleic acid molecule comprises or results from an ABL1 breakpoint within any of exons 1-4 and 10, or introns 4-5, of ABL1;
(c) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(d) a NUP214 gene, or a portion thereof, wherein the cancer is bone marrow leukemia lymphocytic chronic (CLL) or soft tissue liposarcoma;
(e) an AIF1L gene, or a portion thereof, wherein the cancer is soft tissue sarcoma, or ovary serous carcinoma;
(f) an RCSD1 gene, or a portion thereof, wherein the cancer is bone marrow leukemia lymphocytic chronic (CLL); or
(g) an EXOSC2 gene, or a portion thereof, wherein the cancer is peritoneum adenocarcinoma; and
(2) generating a report comprising one or more treatment options identified for the individual based, at least in part, on detection or on acquiring knowledge of the ABL1 fusion nucleic acid molecule, or the ABL1 fusion polypeptide encoded by the ABL1 fusion nucleic acid molecule, in the sample, wherein the one or more treatment options comprise an ABL1-targeted therapy.

3. A method of detecting an ABL1 fusion nucleic acid molecule, the method comprising:
(a) providing a plurality of nucleic acid molecules obtained from a sample from an individual having a cancer, wherein the plurality of nucleic acid molecules comprises nucleic acid molecules corresponding to an ABL1 fusion nucleic acid molecule, wherein the ABL1 fusion nucleic acid molecule comprises a fusion between an ABL1 gene, or a portion thereof, fused to:
(i) any one of genes APOOL, BMP2K, DNAH2, RHEX/C1orf186, EHMT1, MED27, RALGPS1, RFFL, SLC27A4, TSC1, TSPAN18, or ZNF804B, or a portion thereof;
(ii) a second gene, or a portion of a second gene, wherein the ABL1 fusion nucleic acid molecule comprises or results from an ABL1 breakpoint within any of exons 1-4 and 10, or introns 4-5, of ABL1;
(iii) a BCR gene, or a portion thereof, wherein the cancer is lung adenocarcinoma, head and neck squamous cell carcinoma (HNSCC), bone sarcoma, esophagus adenocarcinoma, gastroesophageal junction adenocarcinoma, liver cholangiocarcinoma, pancreas ductal adenocarcinoma, ovary serous carcinoma, or lung squamous cell carcinoma (SCC);
(iv) a NUP214 gene, or a portion thereof, wherein the cancer is bone marrow leukemia lymphocytic chronic (CLL) or soft tissue liposarcoma;
(v) an AIF1L gene, or a portion thereof, wherein the cancer is soft tissue sarcoma, or ovary serous carcinoma;
(vi) an RCSD1 gene, or a portion thereof, wherein the cancer is bone marrow leukemia lymphocytic chronic (CLL); or
(vii) an EXOSC2 gene, or a portion thereof, wherein the cancer is peritoneum adenocarcinoma;
(b) optionally, ligating one or more adapters onto one or more nucleic acid molecules from the plurality of nucleic acid molecules;
(c) optionally, amplifying the one or more ligated nucleic acid molecules from the plurality of nucleic acid molecules;
(d) optionally, capturing amplified nucleic acid molecules from the amplified nucleic acid molecules;
(e) sequencing, by a sequencer, the captured nucleic acid molecules to obtain a plurality of sequence reads that represent the captured nucleic acid molecules, wherein one or more of the plurality of sequence reads correspond to the ABL1 fusion nucleic acid molecule;
(f) analyzing the plurality of sequence reads for the presence or absence of the ABL1 fusion nucleic acid molecule; and
(g) based on the analyzing step, detecting the presence or absence of the ABL1 fusion nucleic acid molecule in the sample.

4. The method of claim 1, wherein:
(1) the ABL1 fusion nucleic acid molecule comprises a fusion between an ABL1 gene, or a portion thereof, fused to an APOOL gene, or a portion thereof, and wherein the ABL1 fusion nucleic acid molecule:
(a) comprises, in 5′ to 3′ direction, an APOOL gene, or a portion thereof, fused to an ABL1 gene, or a portion thereof;
(b) comprises or results from a breakpoint within intron 2 of an APOOL gene, and/or exon 2 of an ABL1 gene;
(c) comprises a fusion between exon 2, or a portion thereof, of an APOOL gene fused to exon 2, or a portion thereof, of an ABL1 gene;
(d) comprises or results from a 5′ breakpoint within chromosomal coordinates chrX: 84302705-84302904 and/or a 3′ breakpoint within chromosomal coordinates chr9: 133729584-133729766; and/or
(e) comprises, in 5′ to 3′ direction, exon 1 and exon 2, or a portion thereof, of an APOOL gene, fused to exon 2, or a portion thereof, and exons 3-11 of an ABL1 gene;
optionally, wherein the cancer is a lung adenocarcinoma;
(2) the ABL1 fusion nucleic acid molecule comprises a fusion between an ABL1 gene, or a portion thereof, fused to an BMP2K gene, or a portion thereof, and wherein the ABL1 fusion nucleic acid molecule:
(a) comprises, in 5′ to 3′ direction, a BMP2K gene, or a portion thereof, fused to an ABL1 gene, or a portion thereof;
(b) comprises a fusion between exon 15, or a portion thereof, of a BMP2K gene fused to exon 3, or a portion thereof, of an ABL1 gene;
(c) comprises or results from a 5′ breakpoint within chromosomal coordinates chr4: 79800022-79800062 and/or a 3′ breakpoint within chromosomal coordinates chr9: 133730205-133730245; and/or
(d) comprises, in 5′ to 3′ direction, exons 1-14 and exon 15, or a portion thereof, of a BMP2K gene, fused to exon 3, or a portion thereof, and exons 4-11 of an ABL1 gene;
optionally, wherein the cancer is a bone marrow leukemia T cell acute (T-ALL);
(3) the ABL1 fusion nucleic acid molecule comprises a fusion between an ABL1 gene, or a portion thereof, fused to a DNAH2 gene, or a portion thereof, and wherein the ABL1 fusion nucleic acid molecule:
(a) comprises, in 5′ to 3′ direction, an ABL1 gene, or a portion thereof, fused to a DNAH2 gene, or a portion thereof;
(b) comprises a fusion between exon 10, or a portion thereof, of an ABL1 gene fused to exon 4, or a portion thereof, of a DNAH2 gene;
(c) comprises or results from a 5′ breakpoint within chromosomal coordinates chr9: 133755953-133755993 and/or a 3′ breakpoint within chromosomal coordinates chr17: 7636399-7636439; and/or
(d) comprises, in 5′ to 3′ direction, exons 1-9 and exon 10, or a portion thereof, of an ABL1 gene, fused to exon 4, or a portion thereof, and exons 5-85 of a DNAH2 gene;
optionally, wherein the cancer is a soft tissue sarcoma, and optionally wherein the soft tissue sarcoma is not otherwise specified (NOS);
(4) the ABL1 fusion nucleic acid molecule comprises a fusion between an ABL1 gene, or a portion thereof, fused to a RHEX/Clorf186 gene, or a portion thereof, and wherein the ABL1 fusion nucleic acid molecule:
(a) comprises, in 5′ to 3′ direction a RHEX/Clorf186 gene, or a portion thereof, fused to an ABL1 gene, or a portion thereof;
(b) comprises a fusion between exon 1, or a portion thereof, of a RHEX/Clorf186 gene fused to exon 2, or a portion thereof, of an ABL1 gene;
(c) comprises or results from a 5′ breakpoint within chromosomal coordinates chr1: 206288099-206288139 and/or a 3′ breakpoint within chromosomal coordinates chr9: 133729488-133729528; and/or
(d) comprises, in 5′ to 3′ direction, exon 1, or a portion thereof, of an RHEX/Clorf186 gene, fused to exon 2, or a portion thereof, and exons 3-11 of ABL1;
optionally, wherein the cancer is a bone marrow multiple myeloma;
(5) the ABL1 fusion nucleic acid molecule comprises a fusion between an ABL1 gene, or a portion thereof, fused to an EHMT1 gene, or a portion thereof, and wherein the ABL1 fusion nucleic acid molecule:
(a) comprises, in 5′ to 3′ direction, an EHMT1 gene, or a portion thereof, fused to an ABL1 gene, or a portion thereof;
(b) comprises a fusion between exon 4, or a portion thereof, of an EHMT1 gene fused to exon 2, or a portion thereof, of an ABL1 gene;
(c) comprises or results from a breakpoint within intron 4 of an EHMT1 gene and/or a breakpoint within intron 1 of an ABL1 gene;
(d) comprises or results from a 5′ breakpoint within chromosomal coordinates chr9: 140630475-140630606 and/or a 3′ breakpoint within chromosomal coordinates chr9: 133729408-133729648;
(e) comprises, in 5′ to 3′ direction, exons 1-3 and exon 4, or a portion thereof, of an EHMT1 gene, fused to exon 2, or a portion thereof, and exons 3-11 of an ABL1 gene;
(f) comprises the nucleotide sequence of SEQ ID NO: 2, or a nucleotide sequence having at least about 70% homology thereto; and/or
(g) encodes an ABL1 fusion polypeptide comprising the amino acid sequence of SEQ ID NO: 23, or an amino acid sequence having at least about 70% homology thereto;
optionally, wherein the cancer is a lung adenocarcinoma;
(6) the ABL1 fusion nucleic acid molecule comprises a fusion between an ABL1 gene, or a portion thereof, fused to a MED27 gene, or a portion thereof, and wherein the ABL1 fusion nucleic acid molecule:
(a) comprises, in 5′ to 3′ direction a MED27 gene, or a portion thereof, fused to an ABL1 gene, or a portion thereof;
(b) comprises a fusion between exon 4, or a portion thereof, of a MED27 gene fused to exon 5, or a portion thereof, of an ABL1 gene;
(c) comprises or results from a breakpoint within intron 4 of a MED27 gene and/or a breakpoint within intron 4 of an ABL1 gene;
(d) comprises or results from a 5′ breakpoint within chromosomal coordinates chr9: 134774423-134776006 and/or a 3′ breakpoint within chromosomal coordinates chr9: 133747364-133747762;
(e) comprises, in 5′ to 3′ direction, exons 1-3 and exon 4, or a portion thereof, of a MED27 gene fused to exon 5, or a portion thereof, and exons 6-11 of an ABL1 gene;
(f) comprises the nucleotide sequence of SEQ ID NO: 3, or a nucleotide sequence having at least about 70% homology thereto; and/or
(g) encodes an ABL1 fusion polypeptide comprising the amino acid sequence of SEQ ID NO: 24, or an amino acid sequence having at least about 70% homology thereto;
optionally, wherein the cancer is an ovary epithelial carcinoma, and optionally wherein the ovary epithelial carcinoma is not otherwise specified (NOS);
(7) the ABL1 fusion nucleic acid molecule comprises a fusion between an ABL1 gene, or a portion thereof, fused to a RALGPS1 gene, or a portion thereof, and wherein the ABL1 fusion nucleic acid molecule:
(a) comprises, in 5′ to 3′ direction, a RALGPS1 gene, or a portion thereof, fused to an ABL1 gene, or a portion thereof;
(b) comprises a fusion between exon 1, or a portion thereof, of a RALGPS1 gene fused to exon 3, or a portion thereof, of an ABL1 gene;
(c) comprises or results from a breakpoint within intron 1 of a RALGPS1 gene and/or a breakpoint within intron 2 of an ABL1 gene;
(d) comprises or results from a 5′ breakpoint within chromosomal coordinates chr9: 129694023-129694282 and/or a 3′ breakpoint within chromosomal coordinates chr9: 133730118-133730529; and/or
(e) comprises, in 5′ to 3′ direction, exon 1, or a portion thereof of a RALGPS1 gene, fused to exon 3, or a portion thereof, and exons 4-11 of an ABL1 gene;
optionally, wherein the cancer is melanoma, and optionally wherein the melanoma is unknown primary melanoma;
(8) the ABL1 fusion nucleic acid molecule comprises a fusion between an ABL1 gene, or a portion thereof, fused to a RFFL gene, or a portion thereof, and wherein the ABL1 fusion nucleic acid molecule:
(a) comprises, in 5′ to 3′ direction a RFFL gene, or a portion thereof, fused to an ABL1 gene, or a portion thereof;
(b) comprises a fusion between exon 5, or a portion thereof, of a RFFL gene fused to exon 2, or a portion thereof, of an ABL1 gene;
(c) comprises or results from a 5′ breakpoint within chromosomal coordinates chr17: 33343379-33343419 and/or a 3′ breakpoint within chromosomal coordinates chr9: 133729485-133729525;
(d) comprises, in 5′ to 3′ direction, exons 1-4 and exon 5, or a portion thereof, of an RFFL gene fused to exon 2, or a portion thereof, and exons 3-11 of an ABL1 gene;
(e) comprises the nucleotide sequence of SEQ ID NO: 4, or a nucleotide sequence having at least about 70% homology thereto; and/or
(f) encodes an ABL1 fusion polypeptide comprising the amino acid sequence of SEQ ID NO: 25, or an amino acid sequence having at least about 70% homology thereto;
optionally, wherein the cancer is a soft tissue malignant peripheral nerve sheath tumor (MPNST);
(9) the ABL1 fusion nucleic acid molecule comprises a fusion between an ABL1 gene, or a portion thereof, fused to a SLC27A4 gene, or a portion thereof, and wherein the ABL1 fusion nucleic acid molecule:
(a) comprises, in 5′ to 3′ direction, a SLC27A4 gene, or a portion thereof, fused to an ABL1 gene, or a portion thereof;
(b) comprises a fusion between exon 12, or a portion thereof, of a SLC27A4 gene fused to exon 6, or a portion thereof, of an ABL1 gene;
(c) comprises or results from a breakpoint within intron 12 of a SLC27A4 gene and/or a breakpoint within intron 5 of an ABL1 gene;
(d) comprises or results from a 5′ breakpoint within chromosomal coordinates chr9: 131119603-131119801 and/or a 3′ breakpoint within chromosomal coordinates chr9: 133748211-133748501;
(e) comprises, in 5′ to 3′ direction, exons 1-11 and exon 12, or a portion thereof, of a SLC27A4 gene, fused to exon 6, or a portion thereof, and exons 7-11 of an ABL1 gene;
(f) comprises the nucleotide sequence of SEQ ID NO: 5, or a nucleotide sequence having at least about 70% homology thereto; and/or
(g) encodes an ABL1 fusion polypeptide comprising the amino acid sequence of SEQ ID NO: 26, or an amino acid sequence having at least about 70% homology thereto;
optionally, wherein the cancer is a testis germ cell tumor, and optionally wherein the testis germ cell tumor is a mixed testis germ cell tumor;
(10) the ABL1 fusion nucleic acid molecule comprises a fusion between an ABL1 gene, or a portion thereof, fused to a TSC1 gene, or a portion thereof, and wherein the ABL1 fusion nucleic acid molecule:
(a) comprises, in 5′ to 3′ direction, a TSC1 gene, or a portion thereof, fused to an ABL1 gene, or a portion thereof;
(b) comprises a fusion between exon 19, or a portion thereof, of a TSC1 gene fused to exon 2, or a portion thereof, of an ABL1 gene;
(c) comprises or results from a 5′ breakpoint within chromosomal coordinates chr9:135,776,998-135,777,038 and/or a 3′ breakpoint within chromosomal coordinates chr9: 133729491-133729531; and/or
(d) comprises, in 5′ to 3′ direction, exons 1-18 and exon 19, or a portion thereof, of a TSC1 gene, fused to exon 2, or a portion thereof, and exons 3-11 of an ABL1 gene;
optionally, wherein the cancer is a soft tissue malignant peripheral nerve sheath tumor (MPNST);
(11) the ABL1 fusion nucleic acid molecule comprises a fusion between an ABL1 gene, or a portion thereof, fused to a TSPAN18 gene, or a portion thereof, and wherein the ABL1 fusion nucleic acid molecule:
(a) comprises, in 5′ to 3′ direction, a TSPAN18 gene, or a portion thereof, fused to an ABL1 gene, or a portion thereof;
(b) comprises a fusion between exon 2, or a portion thereof, of a TSPAN18 gene fused to exon 2, or a portion thereof, of an ABL1 gene;
(c) comprises or results from a 5′ breakpoint within chromosomal coordinates chr11: 44881929-44881969 and/or a 3′ breakpoint within chromosomal coordinates chr9: 133729517-133729557; and/or
(d) comprises, in 5′ to 3′ direction, exon 1 and exon 2, or a portion thereof, of a TSPAN18 gene, fused to exon 2, or a portion thereof, and exons 3-11 of an ABL1 gene;
optionally, wherein the cancer is a soft tissue osteosarcoma, and optionally wherein the soft tissue osteosarcoma is an extraskeletal soft tissue osteosarcoma; or
(12) the ABL1 fusion nucleic acid molecule comprises a fusion between an ABL1 gene, or a portion thereof, fused to a ZNF804B gene, or a portion thereof, and wherein the ABL1 fusion nucleic acid molecule:
(a) comprises, in 5′ to 3′ direction a ZNF804B gene, or a portion thereof, fused to an ABL1 gene, or a portion thereof;
(b) comprises a fusion between exon 1, or a portion thereof, of a ZNF804B gene fused to exon 2, or a portion thereof, of an ABL1 gene;
(c) comprises or results from a breakpoint within intron 1 of a ZNF804B gene and/or a breakpoint within intron 1 of an ABL1 gene;
(d) comprises or results from a 5′ breakpoint within chromosomal coordinates chr7: 88521265-88521397 and/or a 3′ breakpoint within chromosomal coordinates chr9: 133729414-133729519; and/or
(e) comprises, in 5′ to 3′ direction, exon 1, or a portion thereof, of a ZNF804B gene, fused to exon 2, or a portion thereof, and exons 3-11 of an ABL1 gene;
optionally, wherein the cancer is a melanoma, optionally wherein the melanoma is a skin melanoma.

5. The method of claim 1, wherein:
(1) the ABL1 fusion nucleic acid molecule comprises a fusion between an ABL1 gene, or a portion thereof, fused to a second gene, or a portion thereof, wherein the ABL1 fusion nucleic acid molecule comprises or results from an ABL1 breakpoint within exon 1 of ABL1, and wherein the ABL1 fusion nucleic acid molecule:
(a) comprises, in 5′ to 3′ direction, the second gene, or a portion thereof, fused to exon 1 of an ABL1 gene, or a portion thereof;
(b) comprises a fusion between an exon or an intron, or a portion thereof, of the second gene fused to exon 1, or a portion thereof, of an ABL1 gene;
(c) comprises or results from a breakpoint within chromosomal coordinates chr9: 133589786-133589985; and/or
(d) comprises, in 5′ to 3′ direction, one or more exons, or portions thereof, of the second gene, fused to exon 1, or a portion thereof, and exons 2-11 of ABL1;
optionally wherein the cancer is a peritoneum adenocarcinoma;
(2) the ABL1 fusion nucleic acid molecule comprises a fusion between an ABL1 gene, or a portion thereof, fused to a second gene, or a portion thereof, wherein the ABL1 fusion nucleic acid molecule comprises or results from an ABL1 breakpoint within exon 2 of ABL1, and wherein the ABL1 fusion nucleic acid molecule:
(a) comprises, in 5′ to 3′ direction, the second gene, or a portion thereof, fused to exon 2, or a portion thereof, of an ABL1 gene;
(b) comprises a fusion between an exon or an intron, or a portion thereof, of the second gene fused to exon 2, or a portion thereof, of an ABL1 gene;
(c) comprises or results from a breakpoint within chromosomal coordinates chr9: 133729584-133729766; and/or
(d) comprises, in 5′ to 3′ direction, one or more exons, or portions thereof, of the second gene, fused to exon 2, or a portion thereof, and exons 3-11 of an ABL1 gene;
optionally wherein the cancer is a lung cancer, and optionally wherein the lung cancer is a lung adenocarcinoma;
(3) the ABL1 fusion nucleic acid molecule comprises a fusion between an ABL1 gene, or a portion thereof, fused to a second gene, or a portion thereof, wherein the ABL1 fusion nucleic acid molecule comprises or results from an ABL1 breakpoint within exon 3 of ABL1, and wherein the ABL1 fusion nucleic acid molecule:
(a) comprises, in 5′ to 3′ direction, the second gene, or a portion thereof, fused to exon 3, or a portion thereof, of an ABL1 gene;
(b) comprises a fusion between an exon or an intron, or a portion thereof, of the second gene fused to exon 3, or a portion thereof, of an ABL1 gene;
(c) comprises or results from a breakpoint within chromosomal coordinates chr9: 133730205-133730245, chr9: 133730195-133730235, chr9: 133730203-133730243, chr9: 133730234-133730274 or chr9: 133730218-133730258; and/or
(d) comprises, in 5′ to 3′ direction, one or more exons, or portions thereof, of the second gene, fused to exon 3, or a portion thereof, and exons 4-11 of an ABL1 gene;
optionally wherein the cancer is a bone marrow leukemia, optionally wherein the bone marrow leukemia is a bone marrow leukemia lymphocytic acute (ALL), a bone marrow leukemia B cell acute (B-ALL), or a bone marrow leukemia T cell acute (T-ALL);
(4) the ABL1 fusion nucleic acid molecule comprises a fusion between an ABL1 gene, or a portion thereof, fused to a second gene, or a portion thereof, wherein the ABL1 fusion nucleic acid molecule comprises or results from an ABL1 breakpoint within exon 4 of ABL1, and wherein the ABL1 fusion nucleic acid molecule:
(a) comprises, in 5′ to 3′ direction, the second gene, or a portion thereof, fused to exon 4, or a portion thereof, of an ABL1 gene;
(b) comprises a fusion between an exon or an intron, or a portion thereof, of the second gene fused to exon 4, or a portion thereof, of an ABL1 gene;
(c) comprises or results from a breakpoint within chromosomal coordinates chr9: 133738173-133738213, chr9: 133738222-133738262, or chr9: 133738214-133738254; and/or
(d) comprises, in 5′ to 3′ direction, one or more exons, or portions thereof, of the second gene, fused to exon 4, or a portion thereof, and exons 5-11 of an ABL1 gene;
optionally wherein the cancer is a leukemia, optionally wherein the leukemia is a bone marrow leukemia B cell acute (B-ALL), a B-lymphoblastic leukemia-lymphoma (B-ALL), or a bone marrow leukemia lymphocytic chronic (CLL);
(5) the ABL1 fusion nucleic acid molecule comprises a fusion between an ABL1 gene, or a portion thereof, fused to a second gene, or a portion thereof, wherein the ABL1 fusion nucleic acid molecule comprises or results from an ABL1 breakpoint within exon 10 of ABL1, and wherein the ABL1 fusion nucleic acid molecule:
(a) comprises, in 5′ to 3′ direction, exon 10, or a portion thereof, of an ABL1 gene fused to the second gene, or a portion thereof;
(b) comprises a fusion between an exon or an intron, or a portion thereof, of the second gene fused to exon 10, or a portion thereof, of an ABL1 gene;
(c) comprises or results from a breakpoint within chromosomal coordinates chr9: 133755953-133755993; and/or
(d) comprises, in 5′ to 3′ direction, exons 1-9 and exon 10, or a portion thereof, of an ABL1 gene fused to one or more exons, or portions thereof, of the second gene;
optionally wherein the cancer is soft tissue sarcoma, and optionally wherein the soft tissue sarcoma is not otherwise specified (NOS);
(6) the ABL1 fusion nucleic acid molecule comprises a fusion between an ABL1 gene, or a portion thereof, fused to a second gene, or a portion thereof, wherein the ABL1 fusion nucleic acid molecule comprises or results from an ABL1 breakpoint within intron 4 of an ABL1 gene, and wherein the ABL1 fusion nucleic acid molecule:
(a) comprises, in 5′ to 3′ direction, the second gene, or a portion thereof, fused to exon 5, or a portion thereof, of an ABL1 gene;
(b) comprises a fusion between an exon or an intron of the second gene, or a portion thereof, fused to intron 4, or a portion thereof, of an ABL1 gene;
(c) comprises or results from a breakpoint within chromosomal coordinates chr9: 133747364-133747762; and/or
(d) comprises, in 5′ to 3′ direction, one or more exons, or portions thereof, of the second gene, fused to exon 5, or a portion thereof, and exons 6-11 of an ABL1 gene;
optionally wherein the cancer is an ovary epithelial carcinoma, and optionally wherein the ovary epithelial carcinoma is not otherwise specified (NOS); or
(7) the ABL1 fusion nucleic acid molecule comprises a fusion between an ABL1 gene, or a portion thereof, fused to a second gene, or a portion thereof, wherein the ABL1 fusion nucleic acid molecule comprises or results from an ABL1 breakpoint within intron 5 of an ABL1 gene, and wherein the ABL1 fusion nucleic acid molecule:
(a) comprises, in 5′ to 3′ direction, the second gene, or a portion thereof, fused to exon 6, or a portion thereof, of an ABL1 gene;
(b) comprises a fusion between an exon or an intron, or a portion thereof, of the second gene fused to intron 5, or a portion thereof, of an ABL1 gene;
(c) comprises or results from a breakpoint within chromosomal coordinates chr9: 133748211-133748501; and/or
(d) comprises, in 5′ to 3′ direction, one or more exons, or portions thereof, of the second gene, fused to exon 6, or a portion thereof, and exons 7-11 of an ABL1 gene;
optionally wherein the cancer is a testis germ cell tumor, and optionally wherein the testis germ cell tumor is a mixed testis germ cell tumor.

6. The method of claim 1, wherein:
(1) the ABL1 fusion nucleic acid molecule comprises a fusion between an ABL1 gene, or a portion thereof, fused to a BCR gene, or a portion thereof, wherein the cancer is lung adenocarcinoma, and wherein the ABL1 fusion nucleic acid molecule:
(a) comprises, in 5′ to 3′ direction, a BCR gene, or a portion thereof, fused to an ABL1 gene, or a portion thereof;
(b) comprises:
a fusion between exon 12, or a portion thereof, of a BCR gene fused to exon 2, or a portion thereof, of an ABL1 gene,
a fusion between exon 13, or a portion thereof, of a BCR gene fused to exon 2, or a portion thereof, of an ABL1 gene, or
a fusion between exon 14, or a portion thereof, of a BCR gene fused to exon 2, or a portion thereof, of an ABL1 gene;
(c) comprises or results from a breakpoint within intron 12, intron 13 or intron 14 of a BCR gene, and/or a breakpoint within intron 1 of an ABL1 gene;
(d) comprises or results from:
a 5′ breakpoint within chromosomal coordinates chr22: 23632033-23632321 and/or a 3′ breakpoint within chromosomal coordinates chr9: 133591666-133591902,
a 5′ breakpoint within chromosomal coordinates chr22: 23634537-23634672 and/or a 3′ breakpoint within chromosomal coordinates chr9: 133672815-133673059,
a 5′ breakpoint within chromosomal coordinates chr22: 23634041-23634323 and/or a 3′ breakpoint within chromosomal coordinates chr9: 133708182-133708567,
a 5′ breakpoint within chromosomal coordinates chr22: 23632683-23632948 and/or a 3′ breakpoint within chromosomal coordinates chr9: 133613427-133613626, or
a 5′ breakpoint within chromosomal coordinates chr22: 23631693-23632188 and/or a 3′ breakpoint within chromosomal coordinates chr9: 133607683-133608245;
(e) comprises, in 5′ to 3′ direction:
exons 1-11 and exon 12, or a portion thereof, of a BCR gene fused to exon 2, or a portion thereof, and exons 3-11 of an ABL1 gene,
exons 1-12 and exon 13, or a portion thereof, of a BCR gene fused to exon 2, or a portion thereof, and exons 3-11 of an ABL1 gene, or
exons 1-13 and exon 14, or a portion thereof, of a BCR gene, fused to exon 2, or a portion thereof, and exons 3-11 of an ABL1 gene;
(f) comprises the nucleotide sequence of any of SEQ ID NOs: 6-10, or a nucleotide sequence having at least about 70% homology thereto; and/or
(g) encodes an ABL1 fusion polypeptide comprising the amino acid sequence of any of SEQ ID NOs: 27-31, or an amino acid sequence having at least about 70% homology thereto;
(2) the ABL1 fusion nucleic acid molecule comprises a fusion between an ABL1 gene, or a portion thereof, fused to a BCR gene, or a portion thereof, wherein the cancer is head and neck squamous cell carcinoma (HNSCC), and wherein the ABL1 fusion nucleic acid molecule:
(a) comprises, in 5′ to 3′ direction, a BCR gene, or a portion thereof, fused to an ABL1 gene, or a portion thereof;
(b) comprises a fusion between exon 13 or a portion thereof, or exon 14 or a portion thereof, of a BCR gene fused to exon 2, or a portion thereof, of an ABL1 gene;
(c) comprises or results from a breakpoint within intron 13 or intron 14 of a BCR gene and/or a breakpoint within intron 1 of an ABL1 gene;
(d) comprises or results from:
a 5′ breakpoint within chromosomal coordinates chr22: 23634011-23634460 and/or a 3′ breakpoint within chromosomal coordinates chr9: 133645693-133646017,
a 5′ breakpoint within chromosomal coordinates chr22: 23634211-23634632 and/or a 3′ breakpoint within chromosomal coordinates chr9: 133619101-133619395,
a 5′ breakpoint within chromosomal coordinates chr22: 23631687-23632492 and/or a 3′ breakpoint within chromosomal coordinates chr9: 133604425-133604577, or
a 5′ breakpoint within chromosomal coordinates chr22: 23632288-23632656 and/or a 3′ breakpoint within chromosomal coordinates chr9: 133663291-133663682;
(e) comprises, in 5′ to 3′ direction:
exons 1-13 and exon 14, or a portion thereof, of a BCR gene, fused to exon 2, or a portion thereof, and exons 3-11 of an ABL1 gene, or
exons 1-12 and exon 13, or a portion thereof, of a BCR gene, fused to exon 2, or a portion thereof, and exons 3-11 of ABL1;
(f) comprises the nucleotide sequence of any of SEQ ID NOs: 11-12, or a nucleotide sequence having at least about 70% homology thereto; and/or
(g) encodes an ABL1 fusion polypeptide comprising the amino acid sequence of any of SEQ ID NOs: 32-33, or an amino acid sequence having at least about 70% homology thereto;
(3) the ABL1 fusion nucleic acid molecule comprises a fusion between an ABL1 gene, or a portion thereof, fused to a BCR gene, or a portion thereof, wherein the cancer is bone sarcoma, and wherein the ABL1 fusion nucleic acid molecule:
(a) comprises, in 5′ to 3′ direction, a BCR gene, or a portion thereof, fused to an ABL1 gene, or a portion thereof;
(b) comprises a fusion between exon 13, or a portion thereof, of a BCR gene fused to exon 2, or a portion thereof, of an ABL1 gene;
(c) comprises or results from a breakpoint within intron 13 of a BCR gene and/or a breakpoint within intron 1 of an ABL1 gene;
(d) comprises or results from a 5′ breakpoint within chromosomal coordinates chr22: 23631722-23631762 and/or a 3′ breakpoint within chromosomal coordinates chr9: 133729484-133729524;
(e) comprises, in 5′ to 3′ direction, exons 1-12 and exon 13, or a portion thereof, of a BCR gene fused to exon 2, or a portion thereof, and exons 3-11 of an ABL1 gene;
(f) comprises the nucleotide sequence of SEQ ID NO: 13, or a nucleotide sequence having at least about 70% homology thereto; and/or
(g) encodes an ABL1 fusion polypeptide comprising the amino acid sequence of SEQ ID NO: 34, or an amino acid sequence having at least about 70% homology thereto;
optionally wherein the bone sarcoma is not otherwise specified (NOS);
(4) the ABL1 fusion nucleic acid molecule comprises a fusion between an ABL1 gene, or a portion thereof, fused to a BCR gene, or a portion thereof, wherein the cancer is esophagus adenocarcinoma, and wherein the ABL1 fusion nucleic acid molecule:
(a) comprises, in 5′ to 3′ direction a BCR gene, or a portion thereof, fused to an ABL1 gene, or a portion thereof;
(b) comprises a fusion between exon 14, or a portion thereof, of a BCR gene fused to exon 2, or a portion thereof, of an ABL1 gene;
(c) comprises or results from a breakpoint within intron 14 of a BCR gene and/or a breakpoint within intron 1 of an ABL1 gene;
(d) comprises or results from a 5′ breakpoint within chromosomal coordinates chr22: 23633346-23634163 and/or a 3′ breakpoint within chromosomal coordinates chr9: 133609201-133609909;
(e) comprises, in 5′ to 3′ direction, exons 1-13 and exon 14, or a portion thereof, of a BCR gene, fused to exon 2, or a portion thereof, and exons 3-11 of an ABL1 gene;
(f) comprises the nucleotide sequence of SEQ ID NO: 14, or a nucleotide sequence having at least about 70% homology thereto; and/or
(g) encodes an ABL1 fusion polypeptide comprising the amino acid sequence of SEQ ID NO: 35, or an amino acid sequence having at least about 70% homology thereto;
(5) the ABL1 fusion nucleic acid molecule comprises a fusion between an ABL1 gene, or a portion thereof, fused to a BCR gene, or a portion thereof, wherein the cancer is gastroesophageal junction adenocarcinoma, and wherein the ABL1 fusion nucleic acid molecule:
(a) comprises, in 5′ to 3′ direction, a BCR gene, or a portion thereof, fused to an ABL1 gene, or a portion thereof;
(b) comprises a fusion between exon 14, or a portion thereof, of a BCR gene fused to exon 2, or a portion thereof, of an ABL1 gene;
(c) comprises or results from a breakpoint within intron 14 of a BCR gene and/or a breakpoint within intron 1 of an ABL1 gene;
(d) comprises or results from a 5′ breakpoint within chromosomal coordinates chr22: 23633432-23633796 and/or a 3′ breakpoint within chromosomal coordinates chr9: 133623903-133624461;
(e) comprises, in 5′ to 3′ direction, exons 1-13 and exon 14, or a portion thereof, of a BCR gene fused to exon 2, or a portion thereof, and exons 3-11 of an ABL1 gene;
(f) comprises the nucleotide sequence of SEQ ID NO: 15, or a nucleotide sequence having at least about 70% homology thereto; and/or
(g) encodes an ABL1 fusion polypeptide comprising the amino acid sequence of SEQ ID NO: 36, or an amino acid sequence having at least about 70% homology thereto;
(6) the ABL1 fusion nucleic acid molecule comprises a fusion between an ABL1 gene, or a portion thereof, fused to a BCR gene, or a portion thereof, wherein the cancer is liver cholangiocarcinoma, and wherein the ABL1 fusion nucleic acid molecule:
(a) comprises, in 5′ to 3′ direction, a BCR gene, or a portion thereof, fused to an ABL1 gene, or a portion thereof;
(b) comprises a fusion between exon 14, or a portion thereof, of a BCR gene fused to exon 2, or a portion thereof, of an ABL1 gene;
(c) comprises or results from a breakpoint within intron 14 of a BCR gene and/or a breakpoint within intron 1 of an ABL1 gene;
(d) comprises or results from a 5′ breakpoint within chromosomal coordinates chr22: 23633837-23634208 and/or a 3′ breakpoint within chromosomal coordinates chr9: 133665426-133665727;
(e) comprises, in 5′ to 3′ direction, exons 1-13 and exon 14, or a portion thereof, of a BCR gene, fused to exon 2, or a portion thereof, and exons 3-11 of an ABL1 gene;
(f) comprises the nucleotide sequence of SEQ ID NO: 16, or a nucleotide sequence having at least about 70% homology thereto; and/or
(g) encodes an ABL1 fusion polypeptide comprising the amino acid sequence of SEQ ID NO: 37, or an amino acid sequence having at least about 70% homology thereto;
(7) the ABL1 fusion nucleic acid molecule comprises a fusion between an ABL1 gene, or a portion thereof, fused to a BCR gene, or a portion thereof, wherein the cancer is pancreas ductal adenocarcinoma, and wherein the ABL1 fusion nucleic acid molecule:
(a) comprises, in 5′ to 3′ direction a BCR gene, or a portion thereof, fused to an ABL1 gene, or a portion thereof;
(b) comprises a fusion between exon 14, or a portion thereof, of a BCR gene fused to exon 2, or a portion thereof, of an ABL1 gene;
(c) comprises or results from a breakpoint within intron 14 of a BCR gene and/or a breakpoint within intron 1 of an ABL1 gene;
(d) comprises or results from a 5′ breakpoint within chromosomal coordinates chr22: 23632858-23633068 and/or a 3′ breakpoint within chromosomal coordinates chr9: 133701595-133701846;
(e) comprises, in 5′ to 3′ direction, exons 1-13 and exon 14, or a portion thereof, of a BCR gene, fused to exon 2, or a portion thereof, and exons 3-11 of an ABL1 gene;
(f) comprises the nucleotide sequence of SEQ ID NO: 17, or a nucleotide sequence having at least about 70% homology thereto; and/or
(g) encodes an ABL1 fusion polypeptide comprising the amino acid sequence of SEQ ID NO: 38, or an amino acid sequence having at least about 70% homology thereto;
(8) the ABL1 fusion nucleic acid molecule comprises a fusion between an ABL1 gene, or a portion thereof, fused to a BCR gene, or a portion thereof, wherein the cancer is ovary serous carcinoma, and wherein the ABL1 fusion nucleic acid molecule:
(a) comprises, in 5′ to 3′ direction, a BCR gene, or a portion thereof, fused to an ABL1 gene, or a portion thereof;
(b) comprises a fusion between exon 14, or a portion thereof, of a BCR gene fused to exon 2, or a portion thereof, of an ABL1 gene;
(c) comprises or results from a breakpoint within intron 14 of a BCR gene and/or a breakpoint within intron 1 of an ABL1 gene;
(d) comprises or results from a 5′ breakpoint within chromosomal coordinates chr22: 23633208-23633695 and/or a 3′ breakpoint within chromosomal coordinates chr9: 133702617-133702992;
(e) comprises, in 5′ to 3′ direction, exons 1-13 and exon 14, or a portion thereof, of a BCR gene, fused to exon 2, or a portion thereof, and exons 3-11 of an ABL1 gene;
(f) comprises the nucleotide sequence of SEQ ID NO: 18, or a nucleotide sequence having at least about 70% homology thereto; and/or
(g) encodes an ABL1 fusion polypeptide comprising the amino acid sequence of SEQ ID NO: 39, or an amino acid sequence having at least about 70% homology thereto; or
(9) the ABL1 fusion nucleic acid molecule comprises a fusion between an ABL1 gene, or a portion thereof, fused to a BCR gene, or a portion thereof, wherein the cancer is lung squamous cell carcinoma (SCC), and wherein the ABL1 fusion nucleic acid molecule:
(a) comprises, in 5′ to 3′ direction, a BCR gene, or a portion thereof, fused to an ABL1 gene, or a portion thereof;
(b) comprises a fusion between exon 14, or a portion thereof, of a BCR gene fused to exon 2, or a portion thereof, of an ABL1 gene;
(c) comprises or results from a breakpoint within intron 14 of a BCR gene and/or a breakpoint within intron 1 of an ABL1 gene;
(d) comprises or results from:
a 5′ breakpoint within chromosomal coordinates chr22: 23634171-23634647 and/or a 3′ breakpoint within chromosomal coordinates chr9: 133638886-133639512, or
a 5′ breakpoint within chromosomal coordinates chr22: 23633910-23634678 and/or a 3′ breakpoint within chromosomal coordinates chr9: 133724778-133725351; and/or
(e) comprises, in 5′ to 3′ direction, exons 1-13 and exon 14, or a portion thereof, of a BCR gene, fused to exon 2, or a portion thereof, and exons 3-11 of an ABL1 gene.

7. The method of claim 1, wherein:
(1) the ABL1 fusion nucleic acid molecule comprises a fusion between an ABL1 gene, or a portion thereof, fused to a NUP214 gene, or a portion thereof, wherein the cancer is bone marrow leukemia lymphocytic chronic (CLL), and wherein the ABL1 fusion nucleic acid molecule:
(a) comprises, in 5′ to 3′ direction, a NUP214 gene, or a portion thereof, fused to an ABL1 gene, or a portion thereof;
(b) comprises a fusion between exon 34, or a portion thereof, of a NUP214 gene fused to exon 3, or a portion thereof, of an ABL1 gene;
(c) comprises or results from a 5′ breakpoint within chromosomal coordinates chr9: 134106039-134106079 and/or a 3′ breakpoint within chromosomal coordinates chr9: 133730222-133730262;
(d) comprises, in 5′ to 3′ direction, exons 1-33 and exon 34, or a portion thereof, of a NUP214 gene, fused to exon 3, or a portion thereof, and exons 4-11 of an ABL1 gene;
(e) comprises the nucleotide sequence of SEQ ID NO: 19, or a nucleotide sequence having at least about 70% homology thereto; and/or
(f) encodes an ABL1 fusion polypeptide comprising the amino acid sequence of SEQ ID NO: 40, or an amino acid sequence having at least about 70% homology thereto; or
(2) the ABL1 fusion nucleic acid molecule comprises a fusion between an ABL1 gene, or a portion thereof, fused to a NUP214 gene, or a portion thereof, wherein the cancer is soft tissue liposarcoma, and wherein the ABL1 fusion nucleic acid molecule:
(a) comprises, in 5′ to 3′ direction, an ABL1 gene, or a portion thereof, fused to a NUP214 gene, or a portion thereof;
(b) comprises a fusion between exon 1, or a portion thereof, of an ABL1 gene fused to exon 31, or a portion thereof, of a NUP214 gene;
(c) comprises or results from a 5′ breakpoint within chromosomal coordinates chr9: 134090583-134090623 and/or a 3′ breakpoint within chromosomal coordinates chr9: 133710830-133710870; and/or
(d) comprises, in 5′ to 3′ direction, exon 1, or a portion thereof, of an ABL1 gene, fused to exon 31, or a portion thereof, and exons 32-36 of NUP214.

8. The method of claim 1, wherein:
(1) the ABL1 fusion nucleic acid molecule comprises a fusion between an ABL1 gene, or a portion thereof, fused to a AIF1L gene, or a portion thereof, wherein the cancer is soft tissue sarcoma, and wherein the ABL1 fusion nucleic acid molecule:
(a) comprises, in 5′ to 3′ direction, an ABL1 gene, or a portion thereof, fused to an AIF1L gene, or a portion thereof;
(b) comprises a fusion between exon 10, or a portion thereof, of an ABL1 gene fused to exon 4, or a portion thereof, of an AIF1L gene;
(c) comprises or results from a 5′ breakpoint within chromosomal coordinates chr9: 133755952-133755992 and/or a 3′ breakpoint within chromosomal coordinates chr9: 133989965-133990005;
(d) comprises, in 5′ to 3′ direction, exons 1-9 and exon 10, or a portion thereof, of an ABL1 gene, fused to exon 4, or a portion thereof, and exons 5-6 of an AIF1L gene;
(e) comprises the nucleotide sequence of SEQ ID NO: 20, or a nucleotide sequence having at least about 70% homology thereto; and/or
(f) encodes an ABL1 fusion polypeptide comprising the amino acid sequence of SEQ ID NO: 41, or an amino acid sequence having at least about 70% homology thereto;
optionally wherein the soft tissue sarcoma is not otherwise specified (NOS); or
(2) the ABL1 fusion nucleic acid molecule comprises a fusion between an ABL1 gene, or a portion thereof, fused to a AIF1L gene, or a portion thereof, wherein the cancer is ovary serous carcinoma, and wherein the ABL1 fusion nucleic acid molecule:
(a) comprises, in 5′ to 3′ direction, an AIF1L gene, or a portion thereof, fused to an ABL1 gene, or a portion thereof;
(b) comprises a fusion between exon 2, or a portion thereof, of an AIF1L gene fused to exon 2, or a portion thereof, of an ABL1 gene;
(c) comprises or results from a breakpoint within intron 2 of an AIF1L gene and/or a breakpoint within intron 1 of an ABL1 gene;
(d) comprises or results from a 5′ breakpoint within chromosomal coordinates chr9: 133978032-133978137 and/or a 3′ breakpoint within chromosomal coordinates chr9: 133729409-133729618; and/or
(e) comprises, in 5′ to 3′ direction, exon 1 and exon 2, or a portion thereof, of an AIF1L gene, fused to exon 2, or a portion thereof, and exons 3-11 of an ABL1 gene.

9. The method of claim 1, wherein the ABL1 fusion nucleic acid molecule comprises a fusion between an ABL1 gene, or a portion thereof, fused to a RCSD1 gene, or a portion thereof, wherein the cancer is bone marrow leukemia lymphocytic chronic (CLL), and wherein the ABL1 fusion nucleic acid molecule:
(a) comprises, in 5′ to 3′ direction, an RCSD1 gene, or a portion thereof, fused to an ABL1 gene, or a portion thereof;
(b) comprises a fusion between exon 4, or a portion thereof, of an RCSD1 gene fused to exon 4, or a portion thereof, of an ABL1 gene;
(c) comprises or results from a 5′ breakpoint within chromosomal coordinates chr1: 167654668-167654708 and/or a 3′ breakpoint within chromosomal coordinates chr9: 133738173-133738213;
(d) comprises, in 5′ to 3′ direction, exons 1-3 and exon 4, or a portion thereof, of a RCSD1 gene, fused to exon 4, or a portion thereof, and exons 5-11 of an ABL1 gene;
(e) comprises the nucleotide sequence of SEQ ID NO: 21, or a nucleotide sequence having at least about 70% homology thereto; and/or
(f) encodes an ABL1 fusion polypeptide comprising the amino acid sequence of SEQ ID NO: 42, or an amino acid sequence having at least about 70% homology thereto.

10. The method of claim 1, wherein the ABL1 fusion nucleic acid molecule comprises a fusion between an ABL1 gene, or a portion thereof, fused to an EXOSC2 gene, or a portion thereof, wherein the cancer is peritoneum adenocarcinoma, and wherein the ABL1 fusion nucleic acid molecule:
(a) comprises, in 5′ to 3′ direction, an EXOSC2 gene, or a portion thereof, fused to an ABL1 gene, or a portion thereof;
(b) comprises a fusion between exon 9, or a portion thereof, of an EXOSC2 gene fused to exon 1, or a portion thereof, of an ABL1 gene;
(c) comprises or results from a breakpoint within exon 9 of an EXOSC2 gene and/or a breakpoint within exon 1 of an ABL1 gene;
(d) comprises or results from a 5′ breakpoint within chromosomal coordinates chr9: 133579154-133579531 and/or a 3′ breakpoint within chromosomal coordinates chr9: 133589786-133589985; and/or
(e) comprises, in 5′ to 3′ direction, exons 1-8 and exon 9, or a portion thereof, of an EXOSC2 gene, fused to exon 1, or a portion thereof, and exons 2-11 of an ABL1 gene.

11. The method of claim 1, wherein:
(a) the ABL1-targeted therapy comprises one or more of a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer comprising an ABL1 gene fusion or rearrangement, an ABL1-targeted therapy being tested in a clinical trial, a treatment for cancer comprising an ABL1 gene fusion or rearrangement being tested in a clinical trial, or any combination thereof;
(b) the ABL1-targeted therapy is a kinase inhibitor, optionally a tyrosine kinase inhibitor; and/or
(c) the ABL1-targeted therapy comprises one or more of imatinib, nilotinib, dasatinib, ponatinib, bosutinib, asciminib, olverembatinib, radotinib or vodobatinib.

12. The method of claim 1, wherein the ABL1 fusion nucleic acid molecule is detected in the sample by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing.

13. The method of claim 1, wherein the ABL1 fusion polypeptide encoded by the ABL1 fusion nucleic acid molecule is detected in the sample by one or more of: immunoblotting, enzyme linked immunosorbent assay (ELISA), immunohistochemistry, or mass spectrometry.

14. The method of claim 1, wherein the ABL1 fusion polypeptide encoded by the ABL1 fusion nucleic acid molecule comprises an ABL1 kinase domain, or a fragment of an ABL1 kinase domain, having a kinase activity, optionally wherein the kinase activity is constitutive; and/or wherein the ABL1 fusion polypeptide encoded by the ABL1 fusion nucleic acid molecule is oncogenic, optionally wherein the ABL1 fusion polypeptide encoded by the ABL1 fusion nucleic acid molecule promotes cancer cell survival, angiogenesis, cancer cell proliferation, and any combination thereof.

15. The method of claim 1, wherein detecting the ABL1 fusion nucleic acid molecule, or the ABL1 fusion polypeptide encoded by the ABL1 fusion nucleic acid molecule, in the sample comprises detecting a fragment of the ABL1 fusion nucleic acid molecule, or of the ABL1 fusion polypeptide encoded by the ABL1 fusion nucleic acid molecule, comprising a breakpoint or fusion junction.

16. The method of claim 1, wherein: (a) the ABL1 fusion nucleic acid molecule comprises a fusion between an ABL1 gene, or a portion thereof, fused to any one of genes APOOL, BMP2K, DNAH2, RHEX/C1orf186, EHMT1, MED27, RALGPS1, RFFL, SLC27A4, TSC1, TSPAN18, or ZNF804B, or a portion thereof; or (b) the ABL1 fusion nucleic acid molecule comprises a fusion between an ABL1 gene, or a portion thereof, fused to a second gene, or a portion of a second gene, wherein the ABL1 fusion nucleic acid molecule comprises or results from an ABL1 breakpoint within any of exons 1-4 and 10, or introns 4-5, of ABL1; and
wherein the cancer is a carcinoma, a sarcoma, a lymphoma, a leukemia, a myeloma, a germ cell cancer, or a blastoma.

17. The method of claim 16, wherein the cancer is a solid tumor or a hematologic malignancy.

18. The method of claim 16, wherein the cancer is a B cell cancer, multiple myeloma, bone marrow multiple myeloma, melanoma, breast cancer, lung cancer, bronchus cancer, colorectal cancer, prostate cancer, pancreatic cancer, stomach cancer, ovarian cancer, ovary epithelial carcinoma, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, testis germ cell tumor, biliary tract cancer, small bowel cancer, appendix cancer, salivary gland cancer, thyroid gland cancer, adrenal gland cancer, osteosarcoma, soft tissue osteosarcoma, chondrosarcoma, a cancer of hematological tissue, an adenocarcinoma, an inflammatory myofibroblastic tumor, a gastrointestinal stromal tumor (GIST), colon cancer, myelodysplastic syndrome (MDS), myeloproliferative disorder (MPD), acute lymphocytic leukemia (ALL) or bone marrow leukemia lymphocytic acute (ALL), bone marrow leukemia T cell acute (T-ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinoma, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, squamous cell cancer, non-squamous cell cancer, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, lung adenocarcinoma, non-small cell lung cancer, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, peritoneum adenocarcinoma, soft tissue malignant peripheral nerve sheath tumor (MPNST) or a carcinoid tumor.

19. The method of claim 1, wherein the sample: (a) comprises a tissue biopsy sample, a liquid biopsy sample, or a normal control; (b) is from a tumor biopsy, tumor specimen, or circulating tumor cell; (c) is a liquid biopsy sample comprising blood, plasma, cerebrospinal fluid, sputum, stool, urine, or saliva; or (d) comprises cells and/or nucleic acids from the cancer.

20. The method of claim 1, wherein the individual is a human.
